SEQUENCE LISTING (60 sequences)

SEQ ID NO: 1 - DNA, 360 bp, Artificial Sequence
  Description of Artificial Sequence Synthetic humanized antibody
gtcaaactgcagcagtctggggcagagcttgtgaagccaggggcctcagtcaagttgttctgcacagcttctggcttcaacattaaagacacctatatgcactgggtgaagcagaggcctcaacagggcctggagtggattggaaggattgatcctgcgagtggcgatactaaatatgacccgaagttccaggtcaaggccactattacagcggacacgtcctccaacacagcctggctgcagctcagcagcctgacatctgaggacactgccgtctactactgtgcagacggaatgtgggtatcaacgggatatgctctggacttctggggccaagggaccacggtcaccgtctcctca

SEQ ID NO: 2 - protein, 120 aa, Artificial Sequence
  Description of Artificial Sequence Synthetic humanized antibody
VKLQQSGAELVKPGASVKLFCTASGFNIKDTYMHWVKQRPQQGLEWIGRIDPASGDTKYDPKFQVKATITADTSSNTAWLQLSSLTSEDTAVYYCADGMWVSTGYALDFWGQGTTVTVSS

SEQ ID NO: 3 - DNA, 318 bp, Artificial Sequence
  Description of Artificial Sequence Synthetic humanized antibody
agtattgtgatgacccagactcccaaattcctgcttgtttcagcaggagacagggttaccataacctgcaaggccagtcagagtgtgactaatgatgtagcttggtaccaacagaagccagggcagtctcctaaactgctgatatattatgcatccaatcgctacactggagtccctgatcgcttcactggcagtggatatgggacggatttcactttcaccatcagcactgtgcaggctgaagacctggcagtttatttctgtcagcaggattatagctctccgtacacgttcggaggggggaccaagctggagatc

SEQ ID NO: 4 - protein, 106 aa, Artificial Sequence
  Description of Artificial Sequence Synthetic humanized antibody
SIVMTQTPKFLLVSAGDRVTITCKASQSVTNDVAWYQQKPGQSPKLLIYYASNRYTGVPDRFTGSGYGTDFTFTISTVQAEDLAVYFCQQDYSSPYTFGGGTKLEI

SEQ ID NO: 5 - DNA, 429 bp, Artificial Sequence
  Description of Artificial Sequence Synthetic humanized antibody
atggactggacctggagggtcttctgcttgctggctgtagcaccaggtgcccactcccaggtccaactgcaggagtccggtgctgaagttgttaaaccgggttcctccgttaaactgtcctgcaaagcttccggtttcaacatcaaagacacctacatgcactgggttaaacagcgtccgggtcagggtctggaatggatcggtcgtatcgacccggcttccggtgacaccaaatacgacccgaaattccaggttaaagctaccatcaccgctgacgaatccacctccaccgcttacctggaactgtcctccctgcgttccgaagacaccgctgtttactactgcgctgacggtatgtgggtttccaccggttacgctctggacttctggggtcagggtaccacggtcaccgtctcctcaggtgagtcc

SEQ ID NO: 6 - protein, 143 aa, Artificial Sequence
  Description of Artificial Sequence Synthetic humanized antibody
MDWTWRVFCLLAVAPGAHSQVQLQESGAEVVKPGSSVKLSCKASGFNIKDTYMHWVKQRPGQGLEWIGRIDPASGDTKYDPKFQVKATITADESTSTAYLELSSLRSEDTAVYYCADGMWVSTGYALDFWGQGTTVTVSSGES

SEQ ID NO: 7 - DNA, 383 bp, Artificial Sequence
  Description of Artificial Sequence Synthetic humanized antibody
atgggttggtcctgcatcatcctgttcctggttgctaccgctaccggtgttcactccatcgttatgacccagtccccggactccctggctgtttccctgggtgaacgtgttaccatcaactgcaaagcttcccagtccgttaccaacgacgttgcttggtaccagcagaaaccgggtcagtccccgaaactgctgatctactacgcttccaaccgttacaccggtgttccggaccgtttctccggttccggttacggtaccgacttcaccttcaccatctcctccgttcaggctgaagacgttgctgtttactactgccagcaggactactcctccccgtacaccttcggtggtggtaccaaactggagatctaaggatcctc

SEQ ID NO: 8 - protein, 124 aa, Artificial Sequence
  Description of Artificial Sequence Synthetic humanized antibody
MGWSCIILFLVATATGVHSIVMTQSPDSLAVSLGERVTINCKASQSVTNDVAWYQQKPGQSPKLLIYYASNRYTGVPDRFSGSGYGTDFTFTISSVQAEDVAVYYCQQDYSSPYTFGGGTKLEI

SEQ ID NO: 9 - DNA, 429 bp, Artificial Sequence
  Description of Artificial Sequence Synthetic humanized antibody
atggactggacctggagggtcttctgcttgctggctgtagcaccaggtgcccactcccaggtccaactgcaggagagcggtccaggtcttgtgagacctagccagaccctgagcctgacctgcaccgtgtctggcttcaacattaaagacacctatatgcactgggtgagacagccacctggacgaggtcttgagtggattggaaggattgatcctgcgagtggcgatactaaatatgacccgaagttccaggtcagagtgacaatgctggtagacaccagcagcaacacagcctggctgagactcagcagcgtgacagccgccgacaccgcggtctattattgtgcagacggaatgtgggtatcaacgggatatgctctggacttctggggccaagggaccacggtcaccgtctcctcaggtgagtcc

SEQ ID NO: 10 - protein, 143 aa, Artificial Sequence
  Description of Artificial Sequence Synthetic humanized antibody
MDWTWRVFCLLAVAPGAHSQVQLQESGPGLVRPSQTLSLTCTVSGFNIKDTYMHWVRQPPGRGLEWIGRIDPASGDTKYDPKFQVRVTMLVDTSSNTAWLRLSSVTAADTAVYYCADGMWVSTGYALDFWGQGTTVTVSSGES

SEQ ID NO: 11 - DNA, 429 bp, Artificial Sequence
  Description of Artificial Sequence Synthetic humanized antibody
atggactggacctggagggtcttctgcttgctggctgtagcaccaggtgcccactcccaggtccaactgcaggagagcggtccaggtcttgtgagacctagccagaccctgagcctgacctgcaccgtgtctggcttcaacattaaagacacctatatgcactgggtgagacagccacctggacgaggtcttgagtggattggaaggattgatcctgcgagtggcgatactaaatatgacccgaagttccaggtcaaagcgacaattacggcagacaccagcagcaaccagttcagcctgagactcagcagcgtgacagccgccgacaccgcggtctattattgtgcagacggaatgtgggtatcaacgggatatgctctggacttctggggccaagggaccacggtcaccgtctcctcaggtgagtcc

SEQ ID NO: 12 - protein, 143 aa, Artificial Sequence
  Description of Artificial Sequence Synthetic humanized antibody
MDWTWRVFCLLAVAPGAHSQVQLQESGPGLVRPSQTLSLTCTVSGFNIKDTYMHWVRQPPGRGLEWIGRIDPASGDTKYDPKFQVKATITADTSSNQFSLRLSSVTAADTAVYYCADGMWVSTGYALDFWGQGTTVTVSSGES

SEQ ID NO: 13 - DNA, 372 bp, Artificial Sequence
  Description of Artificial Sequence Synthetic humanized antibody
caggtccaactgcaggagagcggtccaggtcttgtgagacctagccagaccctgagcctgacctgcaccgtgtctggcttcaacattaaagacacctatatgcactgggtgagacagccacctggacgaggtcttgagtggattggaaggattgatcctgcgagtggcgatactaaatatgacccgaagttccaggtcagagtgacaatgctggtagacaccagcagcaaccagttcagcctgagactcagcagcgtgacatctgaggacaccgcggtctattattgtgcagacggaatgtgggtatcaacgggatatgctctggacttctggggccaagggaccacggtcaccgtctcctcaggtgagtcc

SEQ ID NO: 14 - protein, 124 aa, Artificial Sequence
  Description of Artificial Sequence Synthetic humanized antibody
QVQLQESGPGLVRPSQTLSLTCTVSGFNIKDTYMHWVRQPPGRGLEWIGRIDPASGDTKYDPKFQVRVTMLVDTSSNQFSLRLSSVTSEDTAVYYCADGMWVSTGYALDFWGQGTTVTVSSGES

SEQ ID NO: 15 - DNA, 429 bp, Artificial Sequence
  Description of Artificial Sequence Synthetic humanized antibody
atggactggacctggagggtcttctgcttgctggctgtagcaccaggtgcccactcccaggtccaactgcaggagagcggtccaggtcttgtgagacctagccagaccctgagcctgacctgcaccgtgtctggcttcaacattaaagacacctatatgcactgggtgaaacagcgacctggacgaggtcttgagtggattggaaggattgatcctgcgagtggcgatactaaatatgacccgaagttccaggtcagagtgacaatgctggtagacaccagcagcaaccagttcagcctgagactcagcagcgtgacagccgccgacaccgcggtctattattgtgcagacggaatgtgggtatcaacgggatatgctctggacttctggggccaagggaccacggtcaccgtctcctcaggtgagtcc

SEQ ID NO: 16 - protein, 143 aa, Artificial Sequence
  Description of Artificial Sequence Synthetic humanized antibody
MDWTWRVFCLLAVAPGAHSQVQLQESGPGLVRPSQTLSLTCTVSGFNIKDTYMHWVKQRPGRGLEWIGRIDPASGDTKYDPKFQVRVTMLVDTSSNQFSLRLSSVTAADTAVYYCADGMWVSTGYALDFWGQGTTVTVSSGES

SEQ ID NO: 17 - DNA, 429 bp, Artificial Sequence
  Description of Artificial Sequence Synthetic humanized antibody
atggactggacctggagggtcttctgcttgctggctgtagcaccaggtgcccactcccaggtccaactgcaggagagcggtccaggtcttgtgagacctagccagaccctgagcctgacctgcaccgcgtctggcttcaacattaaagacacctatatgcactgggtgagacagccacctggacgaggtcttgagtggattggaaggattgatcctgcgagtggcgatactaaatatgacccgaagttccaggtcagagtgacaatgctggtagacaccagcagcaaccagttcagcctgagactcagcagcgtgacagccgccgacaccgcggtctattattgtgcagacggaatgtgggtatcaacgggatatgctctggacttctggggccaagggaccacggtcaccgtctcctcaggtgagtcc

SEQ ID NO: 18 - protein, 143 aa, Artificial Sequence
  Description of Artificial Sequence Synthetic humanized antibody
MDWTWRVFCLLAVAPGAHSQVQLQESGPGLVRPSQTLSLTCTASGFNIKDTYMHWVRQPPGRGLEWIGRIDPASGDTKYDPKFQVRVTMLVDTSSNQFSLRLSSVTAADTAVYYCADGMWVSTGYALDFWGQGTTVTVSSGES

SEQ ID NO: 19 - DNA, 386 bp, Artificial Sequence
  Description of Artificial Sequence Synthetic humanized antibody
atgggttggtcctgcatcatcctgttcctggttgctaccgctaccggtgttcactccgacatccagctgacccagagcccaagcagcctgagcgccagcgtgggtgacagagtgaccatcacctgtaaggccagtcagagtgtgactaatgatgtagcttggtaccagcagaagccaggtaaggctccaaagctgctgatctactatgcatccaatcgctacactggtgtgccaagcagattcagcggtagcggtagcggtaccgacttcaccttcaccatcagcagcctccagccagaggacatcgccacctactactgccagcaggattatagctctccgtacacgttcggccaagggaccaaggtggaaatcaaacgtaagtg

SEQ ID NO: 20 - protein, 128 aa, Artificial Sequence
  Description of Artificial Sequence Synthetic humanized antibody
MGWSCIILFLVATATGVHSDIQLTQSPSSLSASVGDRVTITCKASQSVTNDVAWYQQKPGKAPKLLIYYASNRYTGVPSRFSGSGSGTDFTFTISSLQPEDIATYYCQQDYSSPYTFGQGTKVEIKRK

SEQ ID NO: 21 - DNA, 386 bp, Artificial Sequence
  Description of Artificial Sequence Synthetic humanized antibody
atgggttggtcctgcatcatcctgttcctggttgctaccgctaccggtgtccactccagcatcgtgatgacccagagcccaagcagcctgagcgccagcgtgggtgacagagtgaccatcacctgtaaggccagtcagagtgtgactaatgatgtagcttggtaccagcagaagccaggtaaggctccaaagctgctgatctactatgcatccaatcgctacactggtgtgccagatagattcagcggtagcggttatggtaccgacttcaccttcaccatcagcagcctccagccagaggacatcgccacctactactgccagcaggattatagctctccgtacacgttcggccaagggaccaaggtggaaatcaaacgtaagtg

SEQ ID NO: 22 - protein, 128 aa, Artificial Sequence
  Description of Artificial Sequence Synthetic humanized antibody
MGWSCIILFLVATATGVHSSIVMTQSPSSLSASVGDRVTITCKASQSVTNDVAWYQQKPGKAPKLLIYYASNRYTGVPDRFSGSGYGTDFTFTISSLQPEDIATYYCQQDYSSPYTFGQGTKVEIKRK

SEQ ID NO: 23 - DNA, 386 bp, Artificial Sequence
  Description of Artificial Sequence Synthetic humanized antibody
atgggttggtcctgcatcatcctgttcctggttgctaccgctaccggtgtccactccgacatccagatgacccagagcccaagcagcctgagcgccagcgtgggtgacagagtgaccatcacctgtaaggccagtcagagtgtgactaatgatgtagcttggtaccagcagaagccaggtaaggctccaaagctgctgatctactatgcatccaatcgctacactggtgtgccagatagattcagcggtagcggttatggtaccgacttcaccttcaccatcagcagcctccagccagaggacatcgccacctactactgccagcaggattatagctctccgtacacgttcggccaagggaccaaggtggaaatcaaacgtaagtg

SEQ ID NO: 24 - protein, 128 aa, Artificial Sequence
  Description of Artificial Sequence Synthetic humanized antibody
MGWSCIILFLVATATGVHSDIQMTQSPSSLSASVGDRVTITCKASQSVTNDVAWYQQKPGKAPKLLIYYASNRYTGVPDRFSGSGYGTDFTFTISSLQPEDIATYYCQQDYSSPYTFGQGTKVEIKRK

SEQ ID NO: 25 - DNA, 37 bp, Artificial Sequence
  Description of Artificial Sequence Synthetic primer
cagaaagcttgccgccaccatgagaccgtctattcag

SEQ ID NO: 26 - protein, 6 aa, Artificial Sequence
  Description of Artificial Sequence Synthetic peptide
MRPSIQ

SEQ ID NO: 27 - DNA, 35 bp, Artificial Sequence
  Description of Artificial Sequence Synthetic primer
ccgaggatccactcacgtttgatttccagcttggt

SEQ ID NO: 28 - DNA, 37 bp, Artificial Sequence
  Description of Artificial Sequence Synthetic primer
cagaaagcttgccgccaccatgaaatgcagctgggtc

SEQ ID NO: 29 - protein, 6 aa, Artificial Sequence
  Description of Artificial Sequence Synthetic peptide
MKCSWV

SEQ ID NO: 30 - DNA, 33 bp, Artificial Sequence
  Description of Artificial Sequence Synthetic primer
ccgaggatccactcacctgaggagacggtgact

SEQ ID NO: 31 - DNA, 39 bp, Artificial Sequence
  Description of Artificial Sequence Synthetic primer
gatggtgactctatctcctacagatgcagacagtgagga

SEQ ID NO: 32 - DNA, 32 bp, Artificial Sequence
  Description of Artificial Sequence Synthetic primer
ctgtaggagatagagtcaccatcacttgcaag

SEQ ID NO: 33 - DNA, 39 bp, Artificial Sequence
  Description of Artificial Sequence Synthetic primer
aggagcttttccaggtgtctgttggtaccaagccatata

SEQ ID NO: 34 - DNA, 41 bp, Artificial Sequence
  Description of Artificial Sequence Synthetic primer
accaacagacacctggaaaagctcctaggctgctcatacat

SEQ ID NO: 35 - DNA, 40 bp, Artificial Sequence
  Description of Artificial Sequence Synthetic primer
gcaggctgctgatggtgaaagtataatctctcccagaccc

SEQ ID NO: 36 - DNA, 42 bp, Artificial Sequence
  Description of Artificial Sequence Synthetic primer
actttcaccatcagcagcctgcagcctgaagatattgcaact

SEQ ID NO: 37 - DNA, 59 bp, Artificial Sequence
  Description of Artificial Sequence Synthetic primer
ccgaggatccactcacgtttgatttccaccttggtgccttgaccgaacgtccacagatt

SEQ ID NO: 38 - DNA, 33 bp, Artificial Sequence
  Description of Artificial Sequence Synthetic primer
ggaaaagctcctaggctgctcatatattacaca

SEQ ID NO: 39 - DNA, 38 bp, Artificial Sequence
  Description of Artificial Sequence Synthetic primer
ccgaggatccactcacgtttgatttccacctttgtgcc

SEQ ID NO: 40 - DNA, 51 bp, Artificial Sequence
  Description of Artificial Sequence Synthetic primer
aacccagtgtatataggtgtctttaatgttgaaaccgctagctttacagct

SEQ ID NO: 41 - DNA, 67 bp, Artificial Sequence
  Description of Artificial Sequence Synthetic primer
aaagacacctatatacactgggttagacaggcccctggccaaaggctggagtggatgggaaggattg

SEQ ID NO: 42 - DNA, 26 bp, Artificial Sequence
  Description of Artificial Sequence Synthetic primer
gacccggccctggaacttcgggtcat

SEQ ID NO: 43 - DNA, 66 bp, Artificial Sequence
  Description of Artificial Sequence Synthetic primer
gacccgaagttccagggccgggtcaccatcaccgcagacacctctgccagcaccgcctacatggaa

SEQ ID NO: 44 - DNA, 64 bp, Artificial Sequence
  Description of Artificial Sequence Synthetic primer
ccatagcatagaccccgtagttaccataatatccctctctggcgcagtagtagactgcagtgtc

SEQ ID NO: 45 - DNA, 63 bp, Artificial Sequence
  Description of Artificial Sequence Synthetic primer
ggtaactacggggtctatgctatggactactggggtcaaggaacccttgtcaccgtctcctca

SEQ ID NO: 46 - DNA, 37 bp, Artificial Sequence
  Description of Artificial Sequence Synthetic primer
ccagggccgggtcaccatcaccagagacacctctgcc

SEQ ID NO: 47 - DNA, 27 bp, Artificial Sequence
  Description of Artificial Sequence Synthetic primer
caggcccctggccaagggctggagtgg

SEQ ID NO: 48 - DNA, 17 bp, Artificial Sequence
  Description of Artificial Sequence Synthetic primer
tacgcaaaccgcctctc

SEQ ID NO: 49 - DNA, 18 bp, Artificial Sequence
  Description of Artificial Sequence Synthetic primer
gagtgcaccatatgcggt

SEQ ID NO: 50 - protein, 116 aa, Artificial Sequence
  Description of Artificial Sequence Synthetic humanzied antibody
QVQLVQSGAEVKKPGASVKVSCKASGYSFTSYYIHWVRQAPGQGLEWVGYIDPFNGGTSYNQKFKGKVTMTVDTSTNTAYMELSSLRSEDTAVYYCARGGNRFAYWGQGTLVTVSS

SEQ ID NO: 51 - protein, 106 aa, Mus sp.
DIQMTQSPSSLSASLGGKVTITCKTSQDINKYMAWYQHKPGKRPRLLIHYTSALQPGIPSRFSGSGSGRDYSFNISNLEPEDIATYYCLQYDNLWTFGGGTKLEIK

SEQ ID NO: 52 - protein, 109 aa, Mus sp.
DIQMTQSPSSLSASLGDRVTITCRASQDDISNYLNWYQQKPGGSPKLLIYYASRLHSGVPSRFSGSGSGTDYSLTISNLEQEDIATYFCQQGNTLPPRTFGGGTKLEIK

SEQ ID NO: 53 - protein, 113 aa, Homo sapiens
  MOD_RES (31)..(32): Variable amino acid
DIQMTQSPSSLSASVGDRVTITCRASQSLVXXSISNYLAWYQQKPGKAPKLLIYAASSLESGVPSRFSGSGSGTDFTLTISSLQPEDFATYYCQQYNSLPEWTFGQGTKVEIK

SEQ ID NO: 54 - protein, 107 aa, Homo sapiens
DIQMTQSPSSLSASVGDRVTITCQASQDIIKYLNWYQQTPGKAPKLLIYEASNLQAGVPSRFSGSGSGTDYTPTISSLQPEDIATYYCQQYQSLPYTFGQGTKLQIT

SEQ ID NO: 55 - protein, 105 aa, Artificial Sequence
  Description of Artificial Sequence Synthetic humanized antibody
DQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIK

SEQ ID NO: 56 - protein, 123 aa, Mus sp.
EVQLQQSGAELVKPGASVKLSCTASGFNIKDTYIHCVKQRPEQGLEWIGRIDPANGYTKYDPKFQGKATITADTSSNTAYLQLSSLTSEDTAVYFCAREGYYGNYGVYAMDYWGQGTSVTVSS

SEQ ID NO: 57 - protein, 125 aa, Mus sp.
  MOD_RES (106): Variable amino acid
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCARGYYYYDSXVGYYAMDYWGQGTXVTVSS

SEQ ID NO: 58 - protein, 125 aa, Homo sapiens
QVQLVQSGAEVKKPGASVKVSCKASGYTFTSYAISWVRQAPGQGLEWMGWINPGNGDTNYAQKFQGRVTITADTSTSTAYMELSSLRSEDTAVYYCARAPGYGSGGCYRDYFDYWGQGTLVTVSS

SEQ ID NO: 59 - protein, 120 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGGYYGSGSMNYWGQGTLVTVSS

SEQ ID NO: 60 - protein, 121 aa, Artificial Sequence
  Description of Artificial Sequence Synthetic humanized antibody
QVQLVQSGAEVKKPGASVKVSCKASGFNIKDTYIHWVRQAPGQRLEWMGRIDPANGYTKYDPKFQGRVTITADTSASTAYMELSSLRSEDTAVYYCAEGYYGNYGVYADYWGQGTLVTVSS

(19) United States
(12) Patent Application Publication
(10) Pub. No.: US 2006/0004019 A1
(43) Pub. Date: Jan. 5, 2006

(54) Steroid sparing agents and methods of using same

(76) Inventor: Ivan Lieberburg, Berkeley, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/095,822

(22) Filed: Apr. 1, 2005

Related U.S. Application Data
(60) Provisional application No. 60/558,121, filed on Apr. 1, 2004.

(51) Int. Cl. A61K 31/496

(52) U.S. Cl. 514/253.09

(57) ABSTRACT

This invention relates generally to methods of treatment of inflammatory bowel diseases (IBD), asthma, multiple schlerosis (MS), rheumatoid arthritis (RA), graft vs. host disease (GVHD), host vs. graft disease, and various spondyloarthropathies, comprising administering a steroid sparing immunoglobulin or small molecule composition to a patient in need thereof. The invention also relates generally to combination therapies for the treatment of these conditions.

### FIELD OF THE INVENTION

This invention relates generally to methods of treatment of inflammatory bowel diseases (IBD), asthma, multiple sclerosis (MS), rheumatoid arthritis (RA), graft versus host disease (GVHD), host versus graft disease, and various spondyloarthropathies, comprising administering a steroid sparing immunoglobulin or small molecule composition to a patient in need thereof. The invention also relates generally to combination therapies for the treatment of these conditions.

### BACKGROUND OF THE INVENTION

Inflammation is a response of vascularized tissues to infection or injury and is affected by adhesion of leukocytes to the endothelial cells of blood vessels and their infiltration into the surrounding tissues. In normal inflammation, the infiltrating leukocytes release toxic mediators to kill invading organisms, phagocytize debris and dead cells, and play a role in tissue repair and the immune response. However, in pathologic inflammation, infiltrating leukocytes are over-responsive and can cause serious or fatal damage. See, e.g., Hickey, Psychoneuroimmunology II (Academic Press 1990).

The integrins are a family of cell-surface glycoproteins involved in cell-adhesion, immune cell migration and activation. Alpha-4 integrin is expressed by all circulating leukocytes except neutrophils, and forms heterodimeric receptors in conjunction with either the beta-1 (β₁) or beta-7 (β₇) integrin subunits; both alpha-4 beta-1 (α₄β₁) and alpha-4 beta-7 (α₄β₇) play a role in migration of leukocytes across the vascular endothelium (Springer et al., Cell 1994, 76: 301-14; Butcher et al., Science 1996, 272: 60-6) and contribute to cell activation and survival within the parenchyma (Damle et al., J. Immunol. 1993; 151: 2368-79; Koopman et al., J. Immunol. 1994, 152: 3760-7; Leussink et al., Acta Neuropathol. 2002, 103: 131-136). α₄β₁ is constitutively expressed on lymphocytes, monocytes, macrophages, mast cells, basophils and eosinophils.

α₄β₁ (also known as very late antigen-4, VLA-4), binds to vascular cell adhesion molecule-1 (Lobb et al., J. Clin. Invest. 1994, 94: 1722-8), which is expressed by the vascular endothelium at many sites of chronic inflammation (Bevilacqua et al., 1993 Annu. Rev. Immunol. 11: 767-804; Postigo et al., 1993 Res. Immunol. 144: 723-35). α₄β₁ has other ligands, including fibronectin and other extracellular matrix (ECM) components.

The α₄β₇ dimer interacts with mucosal addressin cell adhesion molecule (MAdCAM-1), and mediates homing of lymphocytes to the gut (Farstad et al., 1997 Am. J. Pathol. 150: 187-99; Issekutz, 1991 J. Immunol. 147: 4178-84). Expression of MAdCAM-1 on the vascular endothelium is also increased at sites of inflammation in the intestinal tract of patients with inflammatory bowel disease (IBD) (Briskin et al., 1997 Am. J. Pathol. 151: 97-110).

Adhesion molecules such as α₄ integrins are potential targets for therapeutic agents. For instance, the VLA-4 receptor of which α₄ integrin is a subunit is an important target because of its interaction with a ligand residing on brain endothelial cells. Diseases and conditions resulting from brain inflammation have particularly severe consequences. In another example, the α₄β₇ integrin dimer is an important target due to its involvement in lymphocyte homing and pathological inflammation in the gastrointestinal tract.

α₄β₁ integrin is expressed on the extracellular surface of activated lymphocytes and monocytes, which have been implicated in the pathogenesis of acute inflammatory brain lesions and blood brain barrier (BBB) breakdown associated with multiple sclerosis (MS) (Coles et al., 1999 Ann. Neurol. 46(3): 296-304). Agents against α₄ integrin have been tested for their anti-inflammatory potential both in vitro and in vivo. See Yednock et al., Nature 1992, 356: 63-66; U.S. Pat. No. 5,840,299 to Bendig et al., issued Nov. 24, 1998, and U.S. Pat. No. 6,001,809 to Thorsett et al., issued Dec. 14, 1999. The in vitro experiments demonstrate that α₄ integrin antibodies block attachment of lymphocytes to brain endothelial cells. Experiments testing the effect of α₄ integrin antibodies on animals having the artificially induced condition simulating multiple sclerosis, experimental autoimmune encephalomyelitis (EAE), have demonstrated that administration of anti-α₄ integrin antibodies prevents inflammation of the brain and subsequent paralysis in the animals. Collectively, these experiments identify anti-α₄ integrin antibodies as potentially useful therapeutic agents for treating multiple sclerosis and other inflammatory diseases and disorders.

Steroids are often indicated for the treatment of inflammatory conditions, but cannot be used safely for extended periods of time. Steroids reduce inflammation, which weakening the immune system. Patients taking steroids may be come dependent, intolerant, or refractory to steroids. Examples of steroids include hydrocortisone, betamethasone, fluorometholone, prednisolone, prednisone, medrysone, dexamethasone, methylprednisolone, rimexolone, and triamcinolone.

Many serious side effects are associated with the use of steroids. The long-term use of steroids is discouraged because of the high risk of long-lasting side effects. Some common side effects include immune suppression, diabetes, weight gain, acne, cataracts, hypertension, psychosis, hirsutism, mood swings, gastritis, muscle weakness, easy bruising, osteroporosis, increased risk of infection and aseptic necrosis. Patients who take steroids for more than two months must often take calcium and vitamin D supplements or other medications, such as biphosphonates, to prevent osteoporosis. Long-term steroid use in children carries the risk of a delay in growth, as well as the side effects that occur in adults.

To date, no therapies have been discovered which allow for safe and effective treatment of inflammatory conditions such as Crohn's disease, asthma, multiple sclerosis (MS), rheumatoid arthritis (RA), graft versus host disease (GVHD), host versus graft disease, and various spondyloarthropathies, without the need for steroids or which allow for the tapering and/or discontinuation of steroids. Steroid sparing agents and methods for using these agents to reduce or eliminate the need for steroids in a subject that is unresponsive, intolerant or dependent on treatment with steroids in statistically significant amount are needed and continue to be sought out for the treatment of inflammatory diseases.

### SUMMARY OF THE INVENTION

Based on the above, new compositions and methods of treating inflammatory diseases involving steroid use are needed which will effectively treat or inhibit these diseases such that patients can achieve long life spans and better quality of life.

This invention relates generally to methods of treatment of inflammatory bowel diseases (IBD), asthma, multiple sclerosis (MS), rheumatoid arthritis (RA), graft versus host disease (GVHD), host versus graft disease, and various spondyloarthropathies, comprising administering an agent which allows steroid use to be reduced or eliminated.

It has been surprisingly discovered that the agents of the present invention are steroid sparing. Steroids are often indicated for the treatment of inflammatory conditions, but cannot be used safely for extended periods of time. Steroids reduce inflammation, which weakening the immune system. Patients taking steroids may be come dependent, intolerant or refractory to steroids.

Accordingly, the agents of the present invention allow for safe and effective treatment of inflammatory conditions such as Crohn's disease, asthma, multiple sclerosis (MS), rheumatoid arthritis (RA), graft versus host disease (GVHD), host versus graft disease, and various spondyloarthropathies, without the need for steroids or which allow for the tapering and/or discontinuation of steroids.

In one embodiment, the steroid sparing agent may be an antibody or an immunologically active fragment thereof, preferably an anti-α₄ immunoglobulin. The antibody or immunologically active fragment thereof is preferably natalizumab (Tysabri®) or an immunologically active fragment thereof. As such, an anti-α₄ immunoglobulin may be administered to a subject for treatment of a disease selected from the group consisting of inflammatory bowel diseases (IBD), asthma, multiple sclerosis (MS), rheumatoid arthritis (RA), graft versus host disease (GVHD), host versus graft disease, and various spondyloarthropathies. When administered in a therapeutically effective amount, the anti-α₄ immunoglobulin permits the subject to be tapered from steroid therapy. Accordingly, it has been surprisingly discovered that when an anti-α₄ immunoglobulin is administered to a subject according to the present invention, the subject requires a therapeutically effective amount of steroids that is less than would be required in the absence of administering the anti-α₄ immunoglobulin.

In another embodiment, the steroid sparing agent may be a small molecule as described herein. As such, the small molecule may be administered to a subject for treatment of a disease selected from the group consisting of inflammatory bowel diseases (IBD), asthma, multiple sclerosis (MS), rheumatoid arthritis (RA), graft versus host disease (GVHD), host versus graft disease, and various spondyloarthropathies. When administered in a therapeutically effective amount, the small molecule, as described herein, permits the subject to be tapered from steroid therapy. Accordingly, it has been surprisingly discovered that when a small molecule, as described herein, is administered to a subject according to the present invention, the subject requires a therapeutically effective amount of steroids that is less than would be required in the absence of administering the compound.

In one embodiment, a method for treating inflammatory bowel disease in a subject is provided, comprising administering to the subject a therapeutically effective amount of a compound, wherein the compound is preferably selected from the group consisting of a compound of formula XXI and a compound of formula XXIa, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

In another embodiment, a method for treating multiple sclerosis in a subject is provided, comprising administering to the subject a therapeutically effective amount of a therapeutically effective amount of a compound wherein the compound is preferably selected from the group consisting of a compound of formula I, a compound of formula I, a compound of formula Ia, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

In another embodiment, a method for treating rheumatoid arthritis in a subject is provided, comprising administering to the subject a therapeutically effective amount of a compound, wherein the compound is preferably selected from the group consisting of a compound of formula I, a compound of formula Ia, a compound of formula II, a compound of formula IIIa, a compound of formula IIIb, a compound of formula IVa, a compound of formula IVb, a compound of formula IVc, a compound of formula IVd, a compound of formula Va, a compound of formula Vb, a compound of formula Vc, a compound of formula Vd, a compound of formula VIa, a compound of formula VIb, a compound of formula VIc, a compound of formula VId, a compound of formula VII, a compound of formula VIII, a compound of formula IX, a compound of formula X, a compound of formula XI, a compound of formula XII, a compound of formula XIII, a compound of formula XIV, a compound of formula XV, a compound of formula XVI, a compound of formula XVII, a compound of formula XVIII, a compound of formula XIX, and a compound of formula XX, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

In another embodiment, a method for treating multiple sclerosis in a subject is provided, comprising administering to the subject a therapeutically effective amount of a compound, wherein the compound is preferably selected from the group consisting of a compound of formula I, a compound of formula Ia, a compound of formula II, a compound of formula VII, a compound of formula VIII, a compound of formula IX, a compound of formula X, a compound of formula XI, a compound of formula XII, a compound of formula XIII, a compound of formula XIV, a compound of formula XV, a compound of formula XVI, a compound of formula XVII, a compound of formula XVIII, a compound of formula XIX, and a compound of formula XX, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

In another embodiment, a method for treating host versus graft or graft versus host in a subject in need thereof is provided, comprising administering to the subject a therapeutically effective amount of a compound wherein the compound is preferably selected from the group consisting of formula I, a compound of formula Ia, a compound of formula II, a compound of formula IIIa, a compound of formula IIIb, a compound of formula IVa, a compound of formula IVb, a compound of formula IVc, a compound of formula IVd, a compound of formula Va, a compound of formula Vb, a compound of formula Vc, a compound of formula Vd, a compound of formula VIa, a compound of formula VIb, a compound of formula VIc, a compound of formula VId, a compound of formula VII, a compound of formula VIII, a compound of formula IX, a compound of formula X, a compound of formula XI, a compound of formula XII, a compound of formula XIII, a compound of formula XIV, a compound of formula XV, a compound of formula XVI, a compound of formula XVII, a compound of formula XVIII, a compound of formula XIX, a compound of formula XX, a compound of formula XXI, and a compound of formula XXIa, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and

c) a combination of a) and b).

In another embodiment, a method for treating asthma in a subject is provided, comprising administering to the subject a therapeutically effective amount of a compound wherein the compound is preferably selected from the group consisting of a compound formula I, a compound of formula Ia, a compound of formula II, a compound of formula IIIa, a compound of formula IIIb, a compound of formula IVa, a compound of formula IVb, a compound of formula IVc, a compound of formula IVd, a compound of formula Va, a compound of formula Vb, a compound of formula Vc, a compound of formula Vd, a compound of formula VIa, a compound of formula VIb, a compound of formula VIc, a compound of formula VId, a compound of formula VII, a compound of formula VIII, a compound of formula IX, a compound of formula X, a compound of formula XI, a compound of formula XII, a compound of formula XIII, a compound of formula XIV, a compound of formula XV, a compound of formula XVI, a compound of formula XVII, a compound of formula XVIII, a compound of formula XIX, and a compound of formula XX, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

In another embodiment, a method for treating spondyloarthropathies in a subject is provided, comprising administering to the subject a therapeutically effective amount of a compound wherein the compound is preferably selected from the group consisting of a compound of formula I, a compound of formula Ia, a compound of formula II, a compound of formula IIIa, a compound of formula IIIb, a compound of formula IVa, a compound of formula IVb, a compound of formula IVc, a compound of formula IVd, a compound of formula Va, a compound of formula Vb, a compound of formula Vc, a compound of formula Vd, a compound of formula VIa, a compound of formula VIb, a compound of formula VIc, a compound of formula VId, a compound of formula VII, a compound of formula VIII, a compound of formula IX, a compound of formula X, a compound of formula XI, a compound of formula XII, a compound of formula XIII, a compound of formula XIV, a compound of formula XV, a compound of formula XVI, a compound of formula XVII, a compound of formula XVIII, a compound of formula XIX, and a compound of formula XX, wherein the amount permits the subject to be tapered from steroid therapy and wherein the subject is selected from the group consisting of:
a) a subject that is unresponsive or intolerant to treatment with immunosuppressive agents;
b) a subject that is unresponsive, intolerant or dependent on treatment with steroids; and
c) a combination of a) and b).

The invention also relates generally to combination therapies for the treatment of these conditions. As such, the steroid sparing agent of the invention can be administered in combination with other steroid sparing agents, as well as in combination with an immunosuppressant, wherein the immunosuppressant is not a steroid, an anti-TNF composition, a 5-ASA composition, and combinations thereof. The steroid sparing agent can be a small molecule as described herein. Alternatively, the steroid sparing agent can be an antibody against VLA-4 or an immunologically active fragment thereof or a polypeptide which binds to VLA-4 thereby preventing it from binding to a cognate ligand.

The invention further relates to a pharmaceutical composition comprising a pharmaceutically acceptable carrier and a therapeutically effective amount of a steroid sparing agent, as disclosed herein, which when administered to a subject in need thereof allows steroid use to be reduced or eliminated.

The compositions of the invention may be administered by a variety of modes of administration including oral, parenteral (e.g., subcutaneous, subdural, intravenous, intramuscular, intrathecal, intraperitoneal, intracerebral, intraarterial, or intralesional routes of administration), topical, localized (e.g., surgical application or surgical suppository), rectal, and pulmonary (e.g., aerosols, inhalation, or powder). Preferably, the compositions of this invention are administered parenterally.

These and other objects, advantages, and features of the invention will become apparent to those persons skilled in the art upon reading the details of the methods and formulations as more fully described below.

### BRIEF DESCRIPTIONS OF THE DRAWINGS

FIG. 1 shows a graph of the response to natalizaumab when given to patients in a Crohn's disease trial.

FIG. 2 shows a graph of the level of remission in response to natalizaumab when given to patients in a Crohn's disease trial.

FIG. 3 shows a graph of the level of remission in response to natalizumab when given to patients in a Crohn's disease trial in various populations: the intention-to-treat population (ITT), elevated C-reactive protein population (CRP), the population unresponsive or intolerant to immunosuppressives (immuno UI). and the population unresponsive, intolerant to, or dependent upon steroids (steroid UID). These categorizations were based upon patient history of previous use of these medications.

### DETAILED DESCRIPTION OF THE INVENTION

Before the present methods and therapeutic agents are described, it is to be understood that this invention is not limited to particular methods and therapeutic agents described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller, subject to any specifically excluded limit in the stated range. Where the stated range includes one or both of the limits, ranges excluding either both of those included limits are also included in the invention. Also contemplated are any values that fall within the cited ranges.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

1. Abbreviations and Definitions

In accordance with this detailed description, the following abbreviations and definitions apply. It must be noted that as used herein, the singular forms “a”, “an”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

1.1 Abbreviations

The following abbreviations have been used herein:
AC acid ceramidase
AcOH acetic acid
ACTH adrenocorticotropic hormone
ANA Anti-nuclear antibodies
aq or aq. aqueous
BBB blood brain barrier
bd broad doublet
bm broad multiplet
Bn benzyl
Boc tert-butoxycarbonyl
Boc₂O di-tert-butyl dicarbonate
BOP benzotriazol-1-yloxy-tris(dimethylamino)phosphonium hexafluorophosphate
bs broad singlet
C constant region of an immunoglobulin
Cbz carbobenzyloxy
CD Crohn's disease
CDAI Crohn's disease activity index
cDNA complementary deoxyribnucleic acid
CDR complementarity determining region
CDR1 complementarity determining region 1
CDR2 complementarity determining region 2
CDR3 complementarity determining region 3
CHCl₃ chloroform
CH₂Cl₂ dichloromethane
CNS central nervous system
(COCl)₂ oxalyl chloride
COX-2 cyclooxygenase-2
CRP C-Reactive Protein
CS Cockayne's syndrome
CSF colony stimulating factor
d doublet
DBU 1,8-diazabicyclo[5.40.0]undec-7-ene
DCC 1,3-dicyclohexylcarbodiimide
dd doublet of doublets
DMAP 4-N,N-dimethylaminopyridine
DME ethylene glycol dimethyl ether
DMF N,N-dimethylformamide
DMSO dimethylsulfoxide
DNA deoxyribonucleic acid
dt doublet of triplets
EAE experimental autoimmune encephalomyelitis
EBNA2 Epstein-Barr virus nuclear antigen 2
ECM extracellular matrix
EDC 1-(3-dimethylaminopropyl)-3-ethylcarbodiimide hydrochloride
EDTA ethylenediaminetetraacetate
ELAMS endothelial adhesion molecules
EM electron microscopy
Et₃N triethylamine
Et₂O diethyl ether
EtOAc ethyl acetate
EtOH ethanol
eq or eq. equivalent
FACS fluorescence activated cell sorter
Fmoc N-(9-fluorenylmethoxycarbonyl)
FmocONSu N-(9-fluorenylmethoxycarbonyl)-Succinimide
FR framework region
FR1 framework region 1
FR2 framework region 2
FR3 framework region 3
g grams
GA glatiramer acetate
GM-CSF granulocyte monocyte colony stimulating factor
GVHD Graft versus host disease
h or hr hour
H heavy chain of an immunoglobulin
HAMA human anti-mouse antibody
HBr hydrobromic acid
HCl hydrochloric acid
H-E hematoxylin-eosin
hex A hexoaminidase A
HIC Hydrophobic interaction chromatography
HIG human immunoglobulin
HMSN IV hereditary motor and sensory neuropathy IV (also known as heredopathia atactica polyneuritiformis)
H₂O water
HOBT 1-hydroxybenzotriazole hydrate
ICAM-1 intercellular adhesion molecule 1
Ig immunoglobulin
IgG immunoglobulin G
IgM immunoglobulin M
IL interleukin
IL-1 interleukin-1
IL-2 interleukin-2
IL-8 interleukin-8
IBD inflammatory bowel disease
IBDQ inflammatory bowel disease questionairre
Immuno UI population unresponsive, intolerant to, or dependent upon immunosuppressives
ITT Intention-to-treat (including all subjects randomized, regardless of whether dosed)
K₂CO₃ potassium carbonate
L light chain of an immunoglobulin
LFA-1 lymphocyte function-related antigen 1—(also known as β₂ integrin, CD11a/CD18 and α_{L}β₂)
m multiplet
MAbs monoclonal antibodies
Mac-1 α_{M}β₂ integrin (also known as CD11b/CD18)
MAdCAM-1 mucosal addressin cell adhesion molecule
MALDI/TOF MS matrix-assisted laser desorption ionization/time-of-flight mass spectrometry
MCP-1 monocyte chemotactic protein 1
MeOH methanol
MES 2-(N-morpholino)ethanesulfonic acid
mg milligram
MgSO₄ magnesium sulfate
min. minute
MIP-1α macrophage inflammatory protein 1 alpha
MIP-1β macrophage inflammatory protein 1 beta
mL milliliter
MLD metachromatic leukodystrophy
mm millimeter
mM millimolar
mmol millimol
mp melting point
MS multiple sclerosis
N normal
NaCl sodium chloride
Na₂CO₃ sodium carbonate
NaHCO₃ sodium bicarbonate
NaOEt sodium ethoxide
NaOH sodium hydroxide
NH₄Cl ammonium chloride
NMM N-methylmorpholine
NSAID nonsteroidal anti-inflammatory
PCR polymerase chain reaction
PEG polyethylene glycol
Phe L-phenylalanine
PKU phenylketonuria
PLP proteolipid protein
PMSF phenylmethylsulfonylfluoride
Pro L-proline
psi pounds per square inch
PtO₂ platinum oxide
q quartet
quint. quintet
RNA ribonucleic acid
rt room temperature
RT-PCR reverse transcription polymerase chain reaction
s singlet
SAE Serious adverse event
SF-36 Quality of Life Question
SAMIs selective adhesion molecule inhibitors
sat or sat. saturated
scFv single chain Fv fragment
SCR solochrome-R-cyanlin
SDS sodium dodecyl sulfate
SDS-PAGE sodium dodecyl sulfate polyacrylamide gel electrophoresis
Steroid UID population unresponsive, intolerant to, or dependent upon steroids
t triplet
t-BuOH tert-butanol
TFA trifluoroacetic acid
TGF-β tumor growth factor beta
THF tetrahydrofuran
TLC or tlc thin layer chromatography
TNF tumor necrosis factor
TNF-α tumor necrosis factor alpha
TNF-β tumor necrosis factor beta
Ts tosyl
TsCl tosyl chloride
TsOH tosylate
UV ultraviolet
VCAM-1 vascular cell adhesion molecule 1
V_{H} heavy chain of the variable domain
V_{L} light chain of the variable domain
VLA-4 very late antigen 4 (also known as alpha-4 beta-1,
α₄β₁)
μL microliter
φ phenyl
   1.2 Definitions

Abbreviations for the twenty naturally occurring amino acids follow conventional usage (IMMOLOGY-A SYNTHESIS (2nd ed., E. S. Golub & D. R. Gren, eds., Sinauer Associates, Sunderland, Mass., 1991)). Stereoisomers (e.g., D-amino acids) of the twenty conventional amino acids, unnatural amino acids such as α,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). Moreover, amino acids may be modified by glycosylation, phosphorylation and the like.

In the polypeptide notation used herein, the left-hand direction is the amino terminal direction and the right-hand direction is the carboxy-terminal direction, in accordance with standard usage and convention. Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

The phrase “polynucleotide sequence” refers to a single or double-stranded polymer of deoxyribonucleotide or ribonucleotide bases read from the 5′ to the 3′ end. It includes self-replicating plasmids, infectious polymers of DNA or RNA and non-functional DNA or RNA.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete DNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Natl. Acad. Sci. (USA) 85: 2444 (1988) (each of which is incorporated by reference in its entirety), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of sequence similarity over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence.

As applied to polypeptides, the term “sequence identity” means peptides share identical amino acids at corresponding positions. The term “sequence similarity” means peptides have identical or similar amino acids (i.e., conservative substitutions) at corresponding positions. The term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. The term “substantial similarity” means that two peptide sequences share corresponding percentages of sequence similarity.

The term “substantially similar” as used herein is intended to mean any polypeptide that has an alteration in the sequence such that a functionally equivalent amino acid is substituted for one or more amino acids in the polypeptide, thus producing a change that has no or relatively little effect on the binding properties of the polypeptide. For example, one or more amino acid residues within the sequence can be substituted by another amino acid of a similar polarity or similar size.

The term “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

For purposes of classifying amino acids substitutions as conservative or non-conservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for another.

Amino acids from the variable regions of the mature heavy and light chains of immunoglobulins are designated Hx and Lxx respectively, where “x” is a number designating the position of an amino acids according to the scheme of Kabat et al., SEQUENCES OF PROTEINS OF IMMUNOLOGICAL INTEREST (National Institutes of Health, Bethesda, Md. (1987) and (1991)) (hereinafter collectively referred to as “Kabat” incorporated by reference in their entirety). Kabat lists many amino acid sequences for antibodies for each subclass, and list the most commonly occurring amino acid for each residue position in that subclass. Kabat uses a method for assigning a residue number to each amino acid in a listed sequence, and this method for assigning residue numbers has become standard in the field. Kabat's scheme is extendible to other antibodies not included in the compendium by aligning the antibody in question with one of the consensus sequences in Kabat. The use of the Kabat numbering system readily identifies amino acids at equivalent positions in different antibodies. For example, an amino acid at the L50 position of a human antibody occupies the equivalence position to an amino acid position L50 of a mouse antibody.

The term “reagent” or “agent” is used to denote a biologically active molecule that binds to a ligand receptor. For example, antibodies or fragments thereof which immunoreact with the VLA-4 receptor or VCAM-1 can eliminate the need for steroids in a subject unresponsive, intolerant or dependent on steroids. Peptides, or peptidomimetics or related compounds, which can act to bind the cell surface receptor, are also contemplated, and can be made synthetically by methods known in the art. Other reagents that react with a VLA-4 receptor as discussed herein or as apparent to those skilled in the art are also contemplated.

A “steroid sparing agent” as used herein refers to any agent that reduces or eliminates the need for steroids in a subject that is unresponsive, intolerant or dependent on treatment with steroids in a statistically significant amount. Preferably, such agents include immunoglobulins (e.g., antibodies, antibody fragments, and recombinantly produced antibodies or fragments), polypeptides (e.g., soluble forms of the ligand proteins for integrins) and small molecules, which when administered in an effective amount, reduces or eliminates the need for steroids in a subject that is unresponsive, intolerant or dependent on treatment with steroids. These agents may be anti-α₄ integrin agents (preferaply anti-α₄β₁ or anti-α₄β₇ antagonists) and anti-VCAM-1 agents. However, with reference to the present invention, such anti-α₄ integrin and anti-VCAM-1 agents only include those which when administered in an effective amount reduce or eliminate the need for steroids in a subject that is unresponsive, intolerant or dependent on treatment with steroids.

The term “anti-α₄ integrin agent” as used herein refers to any agent that binds specifically to an integrin comprising an α₄ subunit and inhibits activity of the integrin.

The term “integrin antagonist” includes any agent that inhibits α₄ subunit-containing integrins from binding with an integrin ligand and/or receptor. Preferably, the integrin antagonist inhibits the α₄ μl dimer an/or the α₄β₇ dimer from binding to its cognate ligand(s). Such antagonists can include anti-integrin antibodies or antibody homolog-containing proteins, as well as other molecules such as soluble forms of the ligand proteins for integrins. Soluble forms of the ligand proteins for α₄ subunit-containing integrins include soluble VCAM-1, VCAM-1 fusion proteins, or bifunctional VCAM-1/Ig fusion proteins. For example, a soluble form of an integrin ligand or a fragment thereof may be administered to bind to integrin, and preferably compete for an integrin binding site on cells, thereby leading to effects similar to the administration of antagonists such as anti-integrin (e.g., VLA-4) antibodies. In particular, soluble integrin mutants that bind ligand but do not elicit integrin-dependent signaling are included within the scope of the invention.

By “natalizumab” or “Tysabri®” is meant a humanized antibody against VLA-4 as described in commonly owned U.S. Pat. Nos. 5,840,299 and 6,033,665, which are herein incorporated by reference in their entirety for all purposes. Also contemplated herein are other VLA-4 specific antibodies. Such steroid sparing antibodies and immunoglobulins include but are not limited to those immunoglobulins described in U.S. Pat. Nos. 6,602,503 and 6,551,593, published U.S. Application No. 20020197233 (Relton. et al.), and as further discussed herein; these patents and applications are incorporated herein by reference in their entirety for all purposes.

The term “efficacy” as used herein in the context of a chronic dosage regime refers to the effectiveness of a particular treatment regime. Efficacy can be measured based on change the course of the disease in response to an agent of the present invention. For example, in the treatment of MS, efficacy can be measured by the frequency of relapses in relapsing-remitting MS, and by the presence or absence of new lesions in the central nervous system as detected using methods such as MRI.

The term “success” as used herein in the context of a chronic treatment regime refers to the effectiveness of a particular treatment regime. This includes a balance of efficacy, toxicity (e.g., side effects and patient tolerance of a formulation or dosage unit), patient compliance, and the like. For a chronic administration regime to be considered “successful” it must balance different aspects of patient care and efficacy to produce the most favorable patient outcome.

The terms “specifically binds” or “binds specifically” as used herein refer to the situation in which one member of a specific binding pair will not show any significant binding to molecules other than its specific binding partner (e.g., an affinity of about 1000× or more for its binding partner). In the present invention, the small compounds, such as N-[N-(3-pyridinesulfonyl)-L-3,3-dimethyl-4-thiaprolyl]-O-[1-methylpiperzain-4-ylcarbonyl]-L-tyrosine isopropyl ester, will not show significant binding to any polypeptide other than an α₄ integrin or a receptor comprising an α₄ integrin. For example, the small compounds used in the methods of the invention that bind to an α₄ integrin with a binding affinity of greater than 0.3 nM are said to bind specifically to an α₄ integrin.

The terms “elicits an immune response” and “elicits a host immune response” as used herein refer to the production of an immune response to a receptor comprising an α₄ integrin in a subject upon introduction of an agent of the invention to the subject. An immune response in the subject can be characterized by a serum reactivity with an α₄ integrin receptor that is at least twice that of an untreated subject, more preferably three times the reactivity of an untreated subject, and even more preferably at least four times the reactivity of an untreated subject, with serum immunoreactivity measured using a serum dilution of approximately 1:100.

“Pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “pharmaceutically-acceptable cation” refers to the cation of a pharmaceutically-acceptable salt.

The term “pharmaceutically acceptable carrier or excipient” is intended to mean any compound used in forming a part of the formulation that is intended to act merely as a carrier, i.e., not intended to have biological activity itself. The pharmaceutically acceptable carrier or excipient is generally safe, non-toxic and neither biologically nor otherwise undesirable. A pharmaceutically acceptable carrier or excipient as used in the specification and claims includes both one and more than one such carrier.

The terms “treating” and “treatment” and the like are used herein to generally mean obtaining a desired pharmacological and physiological effect. More specifically, the reagents described herein are used to reduce or eliminate the need for steroids in a subject that is unresponsive, intolerant or dependent on treatment with steroids. Thus, the effect may be prophylactic in terms of preventing or partially preventing a disease, symptom or condition thereof and/or may be therapeutic in terms of a partial or complete cure of a disease, condition, symptom or adverse effect attributed to the disease depending on the condition or disease being treated. The term “treatment”, as used herein, covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions. The invention is directed towards treating a patient's suffering from disease related to pathological inflammation. The present invention is involved in preventing, inhibiting, or relieving adverse effects attributed to pathological inflammation, preferably an inflammatory bowel disease such as Crohn's disease, asthma, MS, RA or various spondyloarthropathies, over long periods of time and/or are such caused by the physiological responses to inappropriate inflammation present in a biological system over long periods of time.

By “therapeutically effective amount” is meant an amount of agent, reagent, or combination of reagents disclosed herein that when administered to a mammal is sufficient to reduce or eliminate the need for steroids in a subject that is unresponsive, intolerant or dependent on treatment with steroids in statistically significant amount.

By the term “steroid sparing effective amount” is meant an amount of an agent, reagent, or composition effective to that reduces or eliminates the need for steroids in a subject that is unresponsive, intolerant or dependent on treatment with steroids in statistically significant amount. The “steroid sparing effective amount” will vary depending on the compound or composition, the specific disease to be treated and its severity, and the age, weight, etc., of the mammal to be treated.

By “chronic administration” is meant administration of an agent, reagent, or combination therapy of the invention in an amount and periodicity to result in the reduction or the elimination of the need for steroids in a subject that is unresponsive, intolerant or dependent on treatment with steroids. Administration is preferably biweekly, weekly, monthly, or every other month, but can be daily. More preferably the treatment is weekly or monthly and is administered for 6 months to several years or the remainder of the patient's life depending on the disease or condition being treated.

Additional definitions relevant to the compounds of formula I to formula IX are as defined therein.

2. General Aspects of the Invention

2.1 Diseases and Conditions

The following disease and/or conditions may be treated and/or prevented using the steroid sparing agents of the present invention.

2.1.1 Inflammatory Bowel Diseases

Inflammatory bowel disease (IBD) is the general name given to diseases that cause inflammation in the intestines. Inflammatory bowel diseases include Crohn's disease and ulcerative colitis.

2.1.1.1 Crohn's Disease

Crohn's disease causes inflammation in the small intestine. Crohn's disease usually occurs in the lower part of the small intestine, i.e., the illium, but it can affect any part of the digestive tract. The inflammation extends deep into the lining of the affected organ, causing pain and causing the intestines to empty frequently. Crohn's disease may also be called ileitis or enteritis. Crohn's disease affects men and women equally and may run in some families. About 20 percent of people with Crohn's disease have a blood relative with some form of IBD.

The cause of Crohn's disease is uncertain. One theory is that the immune system reacts to a virus or a bacterium by causing ongoing inflammation in the intestine. Patients suffering from Crohn's disease tend to have abnormalities of the immune system, but it is uncertain whether these abnormalities are a cause or result of the disease.

The most common symptoms of Crohn's disease include abdominal pain, often in the lower right area, and diarrhea. Rectal bleeding, weight loss, and fever may also occur. Bleeding may be serious and persistent, leading to anemia. Children with Crohn's disease may suffer from delayed development and stunted growth.

The most common complication of Crohn's disease is blockage of the intestine. Blockage occurs because Crohn's disease causes a thickening of the intestinal wall with swelling and scar tissue, narrowing the intestinal passage. Crohn's disease may also cause sores and ulcers that tunnel through the affected area into surrounding tissues such as the bladder, vagina, or skin. The tunnels, called fistulas, are a common complication and often become infected. Sometimes fistulas can be treated with medication, but often require surgery.

Nutritional complications, such as deficiencies of proteins, calories and vitamins, are common in Crohn's disease. Other complications associated with Crohn's disease include arthritis, skin problems, inflammation in the eyes or mouth, kidney stones, gallstones, or other diseases of the liver and biliary system.

Treatment for Crohn's disease depends on the location and severity of disease, complications, and response to previous treatment. The goals of treatment are to control inflammation, correct nutritional deficiencies, and relieve symptoms such abdominal pain, diarrhea, and rectal bleeding. Treatment may include drugs, nutritional supplements, surgery, or a combination of these options. At this time, treatment there is no cure. Some patients have long periods of remission, free of symptoms. However, Crohn's disease usually recurs at various times over a person's lifetime. Predicting when a remission may occur or when symptoms will return is not possible.

Most patients are first treated with drugs containing mesalamine, a substance that helps control inflammation. Sulfasalazine is also commonly used. Patients who do not benefit from it, or who cannot tolerate it, may be put on other mesalamine-containing drugs, generally known as 5-ASA agents, such as Dipentum®, or Pentasa®. Possible side effects of mesalamine preparations include nausea, vomiting, heartburn, diarrhea, and headache.

Some patients are administered steroids, such as budesonide, to control inflammation. These drugs are the most effective for active Crohn's disease, but they can cause serious side effects, including greater susceptibility to infection. Drugs that suppress the immune system are also used to treat Crohn's disease. The most common include 6-mercaptopurine and azathioprine. Immunosuppressive agents work by blocking the immune reaction that contributes to inflammation. These drugs may cause side effects such as nausea, vomiting, and diarrhea, and lower a patient's resistance to infection.

Antibiotics are used to treat bacterial overgrowth in the small intestine caused by stricture, fistulas, or prior surgery. For this common problem, the doctor may prescribe antibiotics including ampicillin, sulfonamide, cephalosporin, tetracycline, or metronidazole.

Biologics are also used in the treatment of Crohn's disease. Infliximab (Remicade®) is indicated for the treatment of moderate to severe Crohn's disease that does not respond to standard therapies (i.e., mesalamine substances, corticosteroids and immunosuppressive agents) and for the treatment of open, draining fistulas. Infliximab is an anti-tumor necrosis factor (TNF) substance. TNF is a protein produced by the immune system that may cause the inflammation associated with Crohn's disease.

Surgery to remove part of the intestine can help Crohn's disease but cannot cure it. The inflammation tends to return to the area of intestine adjacent to that has been removed. Many Crohn's disease patients require surgery, either to relieve symptoms that do not respond to medical therapy, or to correct complications such as blockage, perforation, abscess or bleeding in the intestine. Some patients must have their entire colon removed by colectomy. See HARRISON'S PRINCIPLES OF INTERNAL MEDICNE; 13^{th} Ed. (1994) McGraw Hill, New York, 1403-1405; THE PHYSICIAN'S DESK REFERENCE; 58^{th} Ed. (2004) Thomson P D R, Montvale, N.J., 402, 1130, 2707, 3153-3155, 3173.

2.1.1.2 Ulcerative Colitis

Ulcerative Colitis is a chronic, inflammatory, and ulcerative disease arising in the colonic mucosa. The cause of ulcerative colitis is unknown. Evidence suggests that a genetic predisposition causes an unregulated intestinal immune response to an environmental, dietary, or infectious agent. However, no inciting antigen has been identified.

Pathologic changes begin with degeneration of the reticulin fibers beneath the mucosal epithelium, occlusion of the subepithelial capillaries, and progressive infiltration of the lamina propria with plasma cells, eosinophils, lymphocytes and mast cells. Crypt abscesses, epithelial necrosis, and mucosal ulceration ultimately develop. The disease usually begins in the rectosigmoid and may extend into the entire colon, or it may involve most of the large bowel.

Symptoms include bloody diarrhea, peritonitis, and profound toxemia. Some cases develop following a documented infection (ie., by amebiasis or bacillary dysentery). Malaise, fever, anemia, anorexia, weight loss, leukocytosis and hypoalbuminemia may be present. Bleeding is the most common local complication. Another severe complication, toxic colitis, occurs when extension of ulceration results in localized ileus and peritonitis. As toxic colitis progresses, the colon loses muscular tone and begins to dilate within hours or days.

Toxic megacolon (or toxic dilation) exists when the diameter of the transverse colon exceeds 6 centimeters, resulting in a high fever, leukocytosis, abdominal pain, and rebound tenderness. Treatment must be given in the early stages to avoid dangerous complications, such as perforation, generalized peritonitis and septicemia. The incidence of colon cancer is increased when the entire colon is involved and the disease lasts for greater than ten years, independent of disease activity. Although cancer incidence is highest in cases of universal ulcerative colitis, the risk is significantly increased with any extent of ulcerative colitis above the sigmoid.

Other complications include peripheral arthritis, ankylosing spondylitis, sacroiliitis, anterior uveitis, erythema nodosum, skin complications, and in children, retarded growth and development. Liver disease may manifest as fatty liver or more seriously as autoimmune hepatitis, primary sclerosing cholangitis, or cirrhosis.

Ulcerative colitis is chronic with repeated exacerbations and remissions. Nearly one third of patients with extensive ulcerative colitis require surgery. Total proctocolectomy is curative: life expectancy and quality of life are restored to normal, and the risk of colon cancer is eliminated.

Ulcerative colitis symptoms may respond to antidiarrheal medications and changes in diet. Moderate to severe symptoms may require one or more medications. For disease in the rectum alone, topical therapy is indicated. Inflammation throughout the colon requires medication that acts on the whole body, such as medications to suppress the immune system (azathioprine, 6-mercaptopurine, or cyclosporine) and to control inflammation (steroids). See HARRISON'S PRINCIPLES OF INTERNAL MEDICINE; 13^{th} Ed. (1994) McGraw Hill, New York, 1403-1405; THE PHYSICIAN'S DESK REFERENCE; 58^{th} Ed. (2004) Thomson P D R, Montvale, N.J., 402, 1130, 2707, 3153-3155, 3173.

2.1.2 Graft Versus Host Disease (GVHD) and Host Versus Graft Disease

Graft versus Host Disease (GVHD) is a rare disorder that can strike persons whose immune system is suppressed and have either received a blood transfusion or a bone marrow transplant. Host versus Graft Disease occurs in patients with suppressed immune systems and who have received an organ transplant. Symptoms for these conditions may include skin rash, intestinal problems similar to colitis, and liver dysfunction.

With GVHD, immunologically competent donor T cells react against antigens in an immunologically depressed recipient. Symptoms of acute GVHD include fever, exfoliative dermatitis, hepatitis with hyperbilirubinemia, vomiting, diarrhea and abdominal pain, which may progress to an ileus, and weight loss. GVHD continues to be the major cause of mortality and severe morbidity after allogeneic bone marrow transplants (BMT).

About ⅓ to ½ of bone marrow transplant recipients develop a chronic form of GVHD. Although the skin, liver, and gut remain the organs primarily affected, other areas of involvement (i.e, joint, lung) are also noted. Ultimately, 20 to 40% of patients die of complications associated with GVHD.

One method of treatment is the removal of T cells from the donor marrow with monoclonal antibodies, using rosetting technique or mechanical separation, before reinfusion of the marrow. T-cell depletion has been very effective in decreasing both the incidence and severity of GVHD. However, the incidences of engraftment failure and relapse are increased. A possible explanation is that the cytokines generated in the graft versus host reaction promote stem cell multiplication and maturation necessary for engraftment. Other agents used to prevent or treat GVHD include methotrexate, corticosteroids, and monoclonal antibodies against antigens expressed on mature T cells.

GVHD may also follow blood transfusions in exceptional cases, because even small numbers of donor T cells can cause GVHD. Such situations include intrauterine fetal blood transfusions and transfusions in immunodepressed patients, such as those with bone marrow transplant recipients, leukemia, lymphoma, neuroblastoma, Hodgkin's and non-Hodgkin's lymphoma See THE MERCK MANUAL OF MEDICAL INFORMATION (1997), Merck Research Laboratories, West Point, Pa., 836-837.

2.1.3 Multiple Sclerosis

Multiple sclerosis (MS) is a chronic neurologic disease, which appears in early adulthood and progresses to a significant disability in most cases. There are approximately 350,000 cases of MS in the United States alone. Outside of trauma, MS is the most frequent cause of neurologic disability in early to middle adulthood.

The cause of MS is yet to be determined. MS is characterized by chronic inflammation, demyelination and gliosis (scarring). Demyelination may result in either negative or positive effects on axonal conduction. Positive conduction abnormalities include slowed axonal conduction, variable conduction block that occurs in the presence of high-but not low-frequency trains of impulses or complete conduction block. Positive conduction abnormalities include ectopic impulse generation, spontaneously or following mechanical stress and abnormal “cross-talk” between demyelinated exons.

T cells reactive against myelin proteins, either myelin basic protein (MBP) or myelin proteolipid protein (PLP) have been observed to mediate CNS inflammation in experimental allergic encephalomyelitis. Patients have also been observed as having elevated levels of CNS immunoglobulin (Ig). It is further possible that some of the tissue damage observed in MS is mediated by cytokine products of activated T cells, macrophages or astrocytes.

Today, 80% patients diagnosed with MS live 20 years after onset of illness. Therapies for managing MS include: (1) treatment aimed at modification of the disease course, including treatment of acute exacerbation and directed to long-term suppression of the disease; (2) treatment of the symptoms of MS; (3) prevention and treatment of medical complications, and (4) management of secondary personal and social problems.

The onset of MS may be dramatic or so mild as to not cause a patient to seek medical attention. The most common symptoms include weakness in one or more limbs, visual blurring due to optic neuritis, sensory disturbances, diplopia and ataxia. The course of disease may be stratified into three general categories: (1) relapsing MS, (2) chronic progressive MS, and (3) inactive MS. Relapsing MS is characterized by recurrent attacks of neurologic dysfunction. MS attacks generally evolve over days to weeks and may be followed by complete, partial or no recovery. Recovery from attacks generally occurs within weeks to several months from the peak of symptoms, although rarely some recovery may continue for 2 or more years.

Chronic progressive MS results in gradually progressive worsening without periods of stabilization or remission. This form develops in patients with a prior history of relapsing MS, although in 20% of patients, no relapses can be recalled. Acute relapses also may occur during the progressive course.

A third form is inactive MS. Inactive MS is characterized by fixed neurologic deficits of variable magnitude. Most patients with inactive MS have an earlier history of relapsing MS.

Disease course is also dependent on the age of the patient. For example, favourable prognostic factors include early onset (excluding childhood), a relapsing course and little residual disability 5 years after onset. By contrast, poor prognosis is associated with a late age of onset (i.e., age 40 or older) and a progressive course. These variables are interdependent, since chronic progressive MS tends to begin at a later age that relapsing MS. Disability from chronic progressive MS is usually due to progressive paraplegia or quadriplegia (paralysis) in patients. In one aspect of the invention, patients will preferably be treated when the patient is in remission rather then in a relapsing stage of the disease.

Short-term use of either adrenocorticotropic hormone or oral corticosteroids (e.g., oral prednisone or intravenous methylprednisolone) is the only specific therapeutic measure for treating patients with acute exacerbation of MS.

Newer therapies for MS include treating the patient with interferon beta-1b, interferon beta-1a, and Copaxone® (formerly known as copolymer 1). These three drugs have been shown to significantly reduce the relapse rate of the disease. These drugs are self-administered intramuscularly or subcutaneously.

2.1.4 Asthma

Asthma is a disease of the respiratory system that involves inflammation of the bronchial tubes, or airways, which carry air to the lungs. The airways overreact to allergens, as well as to smoke, cold air, and/or other environmental factors. The airways narrow, leading to difficulty breathing. Allergens can cause chronic inflammation.

Asthma often develops in childhood or the teen years, and is the most common chronic childhood disease. Most cases of asthma can be controlled. However, in severe cases, asthma episodes can be fatal. The number of cases of asthma has grown steadily in the past 30 years, making it one of the leading public health problems in the United States and the rest of the world.

Asthma is caused by genetic, environmental, and immunological factors, which combine to cause inflammation that can lead to asthma episodes. In some patients, the inflamed airways overreact to substances in the environment, such as smoke or cold air. In other patients, the immune system releases cells that cause inflammation in response to allergens.

Asthma may develop at different times and from a variety of factors. Cigarette smoke and air pollution may cause an attack. In addition, expressions of strong emotions, such as laughing or crying hard, can cause an attack.

Symptoms of an asthma episode can be mild to severe and may include, but are not limited to, wheezing, coughing, chest tightness, rapid, shallow breathing or difficulty breathing, shortness of breath and tiring quickly during exercise.

Treatment involves taking medication to control inflammation and asthma episodes, and avoiding substances that increase inflammation. If inflammation is not controlled, asthma can lead to permanent changes in the bronchial tubes.

Inhaled corticosteroids (such as budesonide and fluticasone), reduce inflammation and are a common treatment for persistent asthma. In rare cases, oral corticosteroids (such as prednisone and dexamethasone) may be used to help control asthma. Long-acting beta2-agonists (such as salmeterol and formoterol) may also be indicated. Medications administered for quick relief include short-acting beta2-agonists (such as albuterol and terbutaline), and anticholinergics (such as ipratropium). See THE MERCK MANUAL OF MEDICAL INFORMATION (1997), Merck Research Laboratories, West Point, Pa., 133-137.

2.1.5 Rheumatoid Arthritis

Rheumatoid Arthritis is a chronic syndrome characterized by inflammation of the peripheral joints, resulting in progressive destruction of articular and periarticular structures. The cause of rheumatoid arthritis is unknown. However, a genetic predisposition has been identified and, in white populations, localized to a pentapeptide in the HLA-DR 1 locus of class II histocompatibility genes. Environmental factors may also play a role. Immunologic changes may be initiated by multiple factors. About 1% of all populations are affected, women two to three times more often than men. Onset may be at any age, most often between 25 and 50 yr.

Prominent immunologic abnormalities that may be important in pathogenesis include immune complexes found in joint fluid cells and in vasculitis. Plasma cells produce antibodies that contribute to these complexes. Lymphocytes that infiltrate the synovial tissue are primarily T helper cells, which can produce pro-inflammatory cytokines. Increased adhesion molecules contribute to inflammatory cell emigration and retention in the synovial tissue.

Rheumatoid nodules occur in up to 30% of patients, usually subcutaneously at sites of chronic irritation. Vasculitis can be found in skin, nerves, or visceral organs in severe cases of RA but is clinically significant in only a few cases.

The onset is usually insidious, with progressive joint involvement, but may be abrupt, with simultaneous inflammation in multiple joints. Tenderness in nearly all inflamed joints and synovial thickening are common. Initial manifestations may occur in any joint.

Stiffness lasting less than 30 minutes on arising in the morning or after prolonged inactivity is common. Subcutaneous rheumatoid nodules are not usually an early manifestation. Visceral nodules, vasculitis causing leg ulcers or mononeuritis multiplex, pleural or pericardial effusions, lymphadenopathy, Felty's syndrome, Sjögren's syndrome, and episcleritis are other manifestations.

As many as 75% of patients improve symptomatically with conservative treatment during the first year of disease. However, less than 10% are eventually severely disabled despite full treatment. The disease greatly affects the lives of most RA patients. Complete bed rest is occasionally indicated for a short period during the most active, painful stage of severe disease. In less severe cases, regular rest should be prescribed.

Nonsteroidal anti-inflammatory drugs may provide important symptomatic relief and may be adequate as simple therapy for mild RA, but they do not appear to alter the long-term course of disease. Salicylates, such as aspirin, may be used for treatment.

Gold compounds usually are given in addition to salicylates or other NSAIDs if the latter do not sufficiently relieve pain or suppress active joint inflammation. In some patients, gold may produce clinical remission and decrease the formation of new bony erosions. Parenteral preparations include gold sodium thiomalate or gold thioglucose. Gold should be discontinued when any of the above manifestations appear. Minor toxic manifestations (e.g., mild pruritus, minor rash) may be eliminated by temporarily withholding gold therapy, then resuming it cautiously about 2 weeks after symptoms have subsided. However, if toxic symptoms progress, gold should be withheld and the patient given a corticosteroid. A topical corticosteroid or oral prednisone 15 to 20 mg/day in divided doses is given for mild gold dermatitis; larger doses may be needed for hematologic complications. A gold chelating drug, dimercaprol 2.5 mg/kg IM, may be given up to four to six times/day for the first 2 days and bid for 5 to 7 days after a severe gold reaction.

Hydroxychloroquine can also control symptoms of mild or moderately active RA. Toxic effects usually are mild and include dermatitis, myopathy, and generally reversible corneal opacity. However, irreversible retinal degeneration has been reported. Sulfasalazine may also be used for treatment of RA.

Oral penicillamine may have a benefit similar to gold and may be used in some cases if gold fails or produces toxicity in patients with active RA. Side effects requiring discontinuation are more common than with gold and include marrow suppression, proteinuria, nephrosis, other serious toxic effects (e.g., myasthenia gravis, pemphigus, Goodpasture's syndrome, polymyositis, a lupuslike syndrome), rash, and a foul taste.

Steroids are the most effective short-term anti-inflammatory drugs. However, their clinical benefit for RA often diminishes with time. Steroids do not predictably prevent the progression of joint destruction. Furthermore, severe rebound follows the withdrawal of corticosteroids in active disease. Contraindications to steroid use include peptic ulcer, hypertension, untreated infections, diabetes mellitus, and glaucoma.

Immunosuppressive drugs are increasingly used in management of severe, active RA. However, major side effects can occur, including liver disease, pneumonitis, bone marrow suppression, and, after long-term use of azathioprine and malignancy.

Joint splinting reduces local inflammation and may relieve severe local symptoms. Active exercise to restore muscle mass and preserve the normal range of joint motion is important as inflammation subsides but should not be fatiguing. Surgery may be performed while the disease is active.

2.1.6 Spondyloarthropathies

The spondyloarthropathies are a family of diseases including ankylosing spondylitis, psoriatic arthritis, Reiter's syndrome, and arthritis associated with inflammatory bowel disease.

2.1.6.1 Ankylosing spondylitis

Ankylosing Spondylitis (AS) is a form of arthritis that is chronic and most often affects the spine. It causes fatigue, pain and stiffness, with swelling and limited motion in the low back, middle back, neck, and hips. Although there is no cure, treatment can usually control symptoms and prevent the condition from getting worse. Complications of ankylosing spondylitis include iritis, difficulty breathing due to curving of the upper body and stiffening of the chest wall.

In time, continued inflammation of the ligaments and joints of the spine causes the spine to fuse together (ankylosis), leading to loss of motion in the neck and low back. As the spine fuses, or stiffens, a fixed bent-forward deformity (kyphosis) can result, leading to significant disability. The inflammation of ankylosing spondylitis can affect other parts of the body, most commonly other joints and the eyes, but sometimes the lungs and heart valves.

Ankylosing spondylitis affects 1 in every 100 people. It is more common in men than in women, and the condition usually begins in the late teens or early adulthood. Treatment includes exercise and physical therapy to help reduce stiffness and maintain good posture and mobility, and medications for pain and inflammation, including steroids.

2.1.6.2 Psoriatic Arthritis

Psoriatic Arthritis (PsA) is characterized by a swelling of the joints that develops in some patients with psoriasis. Psoriatic arthritis displays the symptoms of other types of arthritis, such as stiff, painful and swollen joints. Untreated psoriatic arthritis can cause bone loss and deformation of the joints. The pain and swelling of psoriatic arthritis are caused by an overactive immune system, which enflames the tissues around the joint. Symptoms flare-up and recede periodically. Symmetric arthritis is the most common type of psoriatic arthritis, making up about 50% of all cases. The symptoms occur on both sides of the body. Symptoms are similar to rheumatoid arthritis, and symmetric arthritis can cause permanent damage to the joints. Asymmetric arthritis, the second most common type of psoriatic arthritis, is milder and only causes symptoms on one side of the body.

Distal interphalangeal predominant (DIP), a less common form of psoriatic arthritis, affects the joints close to the fingernails and toenails. The nails are often affected by the condition as well. Spondylitis can make movement painful, especially in the neck and back. It can also cause inflammation of the spinal column. Arthritis mutilans is a frequently debilitating and destructive form of psoriatic arthritis. It often affects the hands and feet, as well as the back and neck, and it can result in permanent deformity.

The symptoms of psoriatic arthritis are similar to those of other kinds of arthritis. They include stiffness in the joints, pain or swelling in the joints, irritation and redness of the eye. The usual symptoms of psoriasis, including red, scaly patches of skin are also present.

Common treatments include nonsteroidal anti-inflammatory drugs (NSAIDs. They include a number of over-the-counter pain medications, such as aspirin and ibuprofen. However, chronic usage of these medications can be dangerous and cause gastrointestinal problems. Cox-2 inhibitors are a class of NSAIDs that are often used to treat psoriatic arthritis. Side effects include nausea and headache.

Immunosuppressants are more powerful drugs that are used for cases of psoriatic arthritis that don't respond to milder medications. Drugs in this class are used for systemic therapy of psoriasis, such as methotrexate, which act by suppressing the immune system. They may also cause serious side effects and raise the risk of infection. Azulfidine is also often prescribed. Certain drugs used to prevent malaria can help with symptoms, and they are sometimes prescribed for psoriatic arthritis as well.

Oral steroids are often indicated to help clear acute joint pain, although steroids cannot be used safely for long periods of time. However, stopping treatment with steroids suddenly can also cause a flare-up of symptoms. Biologics are also used to treat psoriasis. They work by targeting the immune system response that causes the symptoms of psoriasis, preventing the joints from becoming inflamed. Biologic medications may also make the immune system more susceptible to infections.

2.1.6.3 Reiter's Syndrome

Reiter's syndrome, also called reactive arthritis, is a form of arthritis that, in addition to joints, also affects the eyes, urethra and skin.

Reiter's syndrome is characterized by a number of symptoms in different organs of the body that may or may not appear at the same time. The disease may be acute or chronic, with sudden remissions or recurrences. Reiter's syndrome primarily affects males between the ages of 20 and 40. Those with human immunodeficiency virus (HIV) are at a particularly high risk.

The cause of Reiter's syndrome is unknown, but research suggests the disease is caused by a combination of genetic predisposition and other factors. Reiter's syndrome often follows infection with Chlamydia trachomatis or Ureaplasma urealyticum.

The first symptoms of Reiter's syndrome are inflammation of the urethra or the intestines, followed by arthritis. The arthritis usually affects the fingers, toes, ankles, hips, and knee joints. Other symptoms include inflammation of the urethra, with painful urination and discharge, mouth ulcers, inflammation of the eye and Keratoderma blennorrhagica (patches of scaly skin on the palms, soles, trunk, or scalp).

There is no specific treatment for Reiter's syndrome. Joint inflammation is usually treated with nonsteroidal anti-inflammatory drugs (NSAIDs). Skin eruptions and eye inflammation can be treated with steroids. The prognosis for Reiter's syndrome varies. Some patients develop complications that include inflammation of the heart muscle, inflammation with stiffening of the spine, glaucoma, progressive blindness, abnormalities of the feet or accumulation of fluid in the lungs.

Other spondyloarthropathies include, but are not limited to, spondylitis of inflammatory bowel Disease (IBD SpA), Undifferentiated Spondyloarthropathy (uSpA), juvenile spondyloarthropathy (JSpA).

See THE MERCK MANUAL OF MEDICAL INFORMATION (1997), Merck Research Laboratories, West Point, Pa., 243.

3. Administration

In a general sense, the method of the invention does not involve any particular mode of administration, because the mode of administration is dependent upon the form of the active agent and the formulation developed to administer the active agent. Modes of administration include oral, parenteral (e.g., subcutaneous, subdural, intravenous, intramuscular, intrathecal, intraperitoneal, intracerebral, intraarterial, or intralesional routes of administration), topical, localized (e.g., surgical application or surgical suppository), rectal, and pulmonary (e.g., aerosols, inhalation, or powder). Preferably, the route of administration is parenteral. The route of administration is based on the composition being administered (e.g., immunoglobulin being administered intravenously versus small compound being administered orally), tissue targeting (e.g., intrathecal administration to target the site of a spinal cord injury), and the like, as would be known to the artisan of ordinary skill.

Additionally, the immunoglobulins can be combined with other compounds or compositions used to treat, ameliorate or palliate symptoms associated with inflammatory bowel disease such as Crohns's disease, asthma, multiple sclerosis (MS), rheumatoid arthritis (RA), graft versus host disease (GVHD), host versus graft disease, and various spondyloarthropathies. Furthermore, the compounds disclosed herein can be administered alone or in combination with other agents, such as immunosuppresants, 5-ASAs and anti-TNFs. When administered in combination, the immunoglobulins may be administered in the same formulation as these other compounds or compositions, or in a separate formulation, and administered prior to, following, or concurrently with the other compounds and compositions used to treat, ameliorate, or palliate symptoms.

5-aminosalicyclic acid (5-ASAs) are a class of anti-inflammatories commonly used to treat inflammatory bowel disease, such as Crohn's disease and ulcerative colitis. One common 5-ASA is mesalamine, including Pentasa® and Rowasa®. Other 5-ASAs, such as osalazine (Dipentum®) are converted to mesalamine in the body. Sulfasalazine (Azulfidine®) is also commonly administered. Side effects of 5-ASAs include melena, headache, vomiting and rash.

Immunosuppressants weaken or suppress the immune system, which in turn decreases inflammation. Examples include include azathioprine, 6-mercaptopurine, methotrexate, and mycophenolate. These medications are used most often to prevent the body from rejecting a newly transplanted organ, or for inflammatory conditions that have not responded to other treatments. It often takes months for these drugs to improve symptoms, and the disease often returns when medication is discontinued. Side effects of immunosuppressants include nausea, vomiting, diarrhea, stomach ulcers, rash, malaise, liver inflammation, bone marrow suppression, fever, pancreatitis, and increased risk of certain types of cancer.

Anti-TNF agents are also indicated for the treatment of inflammatory conditions. Tumor necrosis factor (TNF) is a protein produced by the immune system that may be related to inflammation. Anti-TNF removes TNF from the bloodstream before it reaches the intestines, thereby preventing inflammation. Infliximab (Remicade®) is an anti-TNF agent indicated for the treatment of moderate to severe Crohn's disease that does not respond to standard therapies (mesalamine substances, corticosteroids, immunosuppressive agents) and for the treatment of open, draining fistulas.

4. Indications for Treatment

Inflammatory diseases that are included for treatment by the compositions, compounds and methods disclosed herein include inflammatory bowel diseases, asthma, multiple sclerosis (MS), rheumatoid arthritis (RA), graft versus host disease (GVHD), host versus graft disease, and various spondyloarthropathies. Additional diseases or conditions contemplated for treatment include those traditionally treated with steroids.

5. Compounds

Various compounds have been identified as agents, which interfere with VLA-4 and VCAM-1 binding. Certain of these compounds, when administered to a patient in an effective amount reduce or eliminate the need for steriod treatment in a subject, preferably a subject with a disease selected from the group consisting of inflammatory bowel disease, asthma, multiple sclerosis, rheumatoid arthritis, graft versus host disease, host versus graft disease, and spondyloarthropathies. Compounds according to the present invention include compounds within formulae I, Ia, and II, described below. In addition, compounds according to the present invention include compounds within formulae IIIa, IIIb, IVa, IVb, IVc, IVd, Va, Vb, Vc, Vd, VIa, VIb, VIc, and VId described below. Compounds according to the present invention further include compounds of formulae VII-XX. Compounds according to the present invention additionally include compounds of formulae XXI and XXIa.

Compounds of Formulae I and II

In one aspect, the compounds that can be utilized as steroid sparing agents for treatment of a subject, with a disease selected from the group consisting of multiple sclerosis, asthma, rheumatoid arthritis, graft versus host disease, host versus graft disease, and spondyloarthropathies, are compounds of formulae I and II. Preferably, the compounds of formulae I and II can be utilized as steriod sparing agents for treatment of a subject with a disease selected from the group consisting of multiple sclerosis, asthma, graft versus host disease, host versus graft disease, and spondyloarthropathies. More preferably, the compounds of formulae I and II can be utilized as steriod sparing agents for treatment of a subject with a disease selected from the group consisting of multiple sclerosis, graft versus host disease, and host versus graft disease.

In one aspect, the compounds that can be utilized as steroid sparing agents are compounds defined by formula I below: 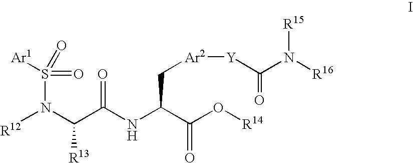
wherein:
Ar¹ is selected from the group consisting of aryl, substituted aryl, heteroaryl, and substituted heteroaryl;
Ar² is selected from the group consisting of aryl, substituted aryl, heteroaryl and substituted heteroaryl;
R¹² and R¹³ together with the nitrogen atom bound to R¹² and the carbon atom bound to R¹³ form a heterocyclic or substituted heterocyclic group;
R¹⁴ is selected from the group consisting of hydrogen, alkyl, substituted alkyl, cycloalkyl, substituted cycloalkyl, aryl, and substituted aryl;
R¹⁵ is selected from the group consisting of alkyl, and substituted alkyl, or R¹⁵ and R¹⁶ together with the nitrogen atom to which they are bound form a heterocyclic or substituted heterocyclic group;
R¹⁶ is selected from the group consisting of alkyl and substituted alkyl or R¹⁵ and R¹⁶ together with the nitrogen atom to which they are bound form a heterocyclic or substituted heterocyclic group; and
Y is selected from the group consisting of —O— and —NR¹⁰⁰—, wherein R¹⁰⁰ is hydrogen or alkyl;
and pharmaceutically acceptable salts thereof.

Preferably, in the compounds of formula I above, R¹⁴ is hydrogen or alkyl.

Preferably, in the compounds of formula I above, Ar¹ is selected from the group consisting of phenyl, 4-methylphenyl, 4-t-butylphenyl, 2,4,6-trimethylphenyl, 2-fluorophenyl, 3-fluorophenyl, 4-fluorophenyl, 2,4-difluorophenyl, 3,4-difluorophenyl, 3,5-difluorophenyl, 2-chlorophenyl, 3-chlorophenyl, 4-chlorophenyl, 3,4-dichlorophenyl, 3,5-dichlorophenyl, 3-chloro-4-fluorophenyl, 4-bromophenyl, 2-methoxyphenyl, 3-methoxyphenyl, 4-methoxyphenyl, 3,4-dimethoxyphenyl, 4-t-butoxyphenyl, 4-(3′-dimethylamino-n-propoxy)-phenyl, 2-carboxyphenyl, 2-(methoxycarbonyl)phenyl, 4-(H₂NC(O)-)phenyl, 4-(H₂NC(S)-)phenyl, 4-cyanophenyl, 4-trifluoromethylphenyl, 4-trifluoromethoxyphenyl, 3,5-di-(trifluoromethyl)phenyl, 4-nitrophenyl, 4-aminophenyl, 4-(CH₃C(O)NH-)phenyl, 4-(PhNHC(O)NH-)phenyl, 4-amidinophenyl, 4-methylamidinophenyl, 4-[CH₃SC(═NH)-]phenyl, 4-chloro-3-[H₂NS(O)₂-]phenyl, 1-naphthyl, 2-naphthyl, pyridin-2-yl, pyridin-3-yl, pyridine-4-yl, pyrimidin-2-yl, quinolin-8-yl, 2-(trifluoroacetyl)-1,2,3,4-tetrahydroisoquinolin-7-yl, 2-thienyl, 5-chloro-2-thienyl, 2,5-dichloro-4-thienyl, 1-N-methylimidazol-4-yl, 1-N-methylpyrazol-3-yl, 1-N-methylpyrazol-4-yl, 1-N-butylpyrazol-4-yl, 1-N-methyl-3-methyl-5-chloropyrazol-4-yl, 1-N-methyl-5-methyl-3-chloropyrazol-4-yl, 2-thiazolyl and 5-methyl-1,3,4-thiadiazol-2-yl.

Preferably, in the compounds of formula I above, R¹² and R¹³ together with the nitrogen atom bound to R¹² and the carbon atom bound to R¹³ form a heterocyclic or substituted heterocyclic of the formula: 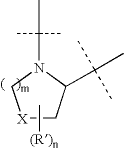
wherein:
X is selected from the group consisting of —C(O)—, —S—, —SO—, —SO₂, and optionally substituted —CH₂—;
m is an integer of 0 to 12;
n is an integer of 0 to 2; and
R′ is selected from the group consisting of alkyl, substituted alkyl, and amino.

Preferably, m is 1, X is —S— or —CH₂—, R¹ is alkyl or substituted alkyl.

Even more preferably, R¹² and R¹³ together with the nitrogen atom bound to R¹² and the carbon atom bound to R¹³ form a heterocyclic or substituted heterocyclic selected from the group consisting of azetidinyl, thiazolidinyl, piperidinyl, piperazinyl, thiomorpholinyl, pyrrolidinyl, 4-hydroxypyrrolidinyl, 4-oxopyrrolidinyl, 4-fluoropyrrolidinyl, 4,4-difluoropyrrolidinyl, 4-(thiomorpholin-4-ylC(O)O-)pyrrolidinyl, 4-[CH₃S(O)₂O-]pyrrolidinyl, 3-phenylpyrrolidinyl, 3-thiophenylpyrrolidinyl, 4-aminopyrrolidinyl, 3-methoxypyrrolidinyl, 4,4-dimethylpyrrolidinyl, 4-N-Cbz-piperazinyl, 4-[CH₃S(O)₂-]piperazinyl, thiazolidin-3-yl, 5,5-dimethyl-thiazolidin-3-yl, 5,5-dimethylthiazolindin-4-yl, 1,1-dioxo-thiazolidinyl, 1,1-dioxo-5,5-dimethylthiazolidin-2-yl and 1,1-dioxothiomorpholinyl.

Preferably, in the compounds of formula I, Ar² is selected from the group consisting of phenyl, 2-pyridyl, 3-pyridyl, 4-pyridyl, and 4-pyrid-2-onyl.

Preferably, in formula I, Y is —O—, and when Y is —O—, the moiety —OC(O)NR¹⁵R¹⁶ is preferably selected from the group consisting of (CH₃)₂NC(O)O—, (piperidin-1-yl)C(O)O—, (4-hydroxypiperidin-1-yl)C(O)O—, (4-formyloxypiperidin-1-yl)C(O)O—, (4-ethoxycarbonylpiperidin-1-yl)C(O)O—, (4-carboxylpiperidin-1-yl)C(O)O—, (3-hydroxymethylpiperidin-1-yl)C(O)O—, (4-hydroxymethylpiperidin-1-yl)C(O)O—, (4-piperidon-1-yl ethylene ketal)C(O)O—, (piperazin-1-yl)-C(O)O—, (1-Boc-piperazin-4-yl)-C(O)O—, (4-methylpiperazin-1-yl)C(O)O—, (4-methylhomopiperazin-1-yl)C(O)O—, (4-(2-hydroxyethyl)piperazin-1-yl)C(O)O—, (4-phenylpiperazin-1-yl)C(O)O—, (4-(pyridin-2-yl)piperazin-1]-yl)C(O)O—, (4-(4-trifluoromethylpyridin-2-yl)piperazin-1-yl)C(O)O—, (4-(pyrimidin-2-yl)piperazin-1-yl)C(O)O—, (4-acetylpiperazin-1-yl)C(O)O—, (4-(phenylC(O)-)piperazin-1-yl)C(O)O—, (4-(pyridin-4′-ylC(O)-)piperazin-1-yl)C(O)O, (4-(phenylNHC(O)-)piperazin-1-yl)C(O)O—, (4-(phenylNHC(S)-)piperazin-1-yl)C(O)O—, (4-methanesulfonylpiperazin-1-yl-C(O)O—, (4-trifluoromethanesulfonylpiperazin-1-yl-C(O)O—, (morpholin-4-yl)C(O)O—, (thiomorpholin-4-yl)C(O)O—, (thiomorpholin-4′-yl sulfone)-C(O)O—, (pyrrolidin-1-yl)C(O)O—, (2-methylpyrrolidin-1-yl)C(O)O—, (2-(methoxycarbonyl)pyrrolidin-1-yl)C(O)O—, (2-(hydroxymethyl)pyrrolidin-1-yl)C(O)O—, (2-(N,N-dimethylamino)ethyl)(CH₃)NC(O)O—, (2-(N-methyl-N-toluene-4-sulfonylamino)ethyl)(CH₃)N—C(O)O—, (2-(morpholin-4-yl)ethyl)(CH₃)NC(O)O—, (2-(hydroxy)ethyl)(CH₃)NC(O)O—, bis(2-(hydroxy)ethyl)NC(O)O—, (2-(formyloxy)ethyl)(CH₃)NC(O)O—, (CH₃₀C(O)CH₂)HNC(O)O—, and 2-[(phenylNHC(O)O-)ethyl-]HNC(O)O—.

Preferred compounds within the scope of formula I include by way of example:
N-(toluene-4-sulfonyl)-L-prolyl-L-4-(4-methylpiperazin-1-ylcarbonyloxy)phenylalanine ethyl ester
N-(toluene-4-sulfonyl)-L-prolyl-L-4-(N,N-dimethylcarbamyloxy)phenylalanine ethyl ester
N-(toluene-4-sulfonyl)-L-prolyl-L-4-(4-methylpiperazin-1-ylcarbonyloxy)phenylalanine isopropyl ester
N-(toluene-4-sulfonyl)-L-prolyl-L-4-(4-methylpiperazin-1-ylcarbonyloxy)phenylalanine n-butyl ester
N-(toluene-4-sulfonyl)-L-prolyl-L-4-(4-methylpiperazin-1-ylcarbonyloxy)phenylalanine cyclopentyl ester
N-(toluene-4-sulfonyl)-L-prolyl-L-4-(4-methylpiperazin-1-ylcarbonyloxy)phenylalanine tert-butyl ester
N-(toluene-4-sulfonyl)-L-prolyl-L-4-(4-methylpiperazin-1-ylcarbonyloxy)phenylalanine
N-(toluene-4-sulfonyl)-L-prolyl-L-4-(N,N-dimethylcarbamyloxy)phenylalanine isopropyl ester
N-(toluene-4-sulfonyl)-L-prolyl-L-4-(N,N-dimethylcarbamyloxy)phenylalanine n-butyl ester
N-(toluene-4-sulfonyl)-L-prolyl-L-4-(N,N-dimethylcarbamyloxy)phenylalanine cyclopentyl ester
N-(toluene-4-sulfonyl)-L-prolyl-L-4-(N,N-dimethylcarbamyloxy)phenylalanine tert-butyl ester
N-(toluene-4-sulfonyl)-L-prolyl-L-4-(N,N-dimethylcarbamyloxy)phenylalanine
N-(α-toluenesulfonyl)-L-prolyl-L-4-(4-methylpiperazin-1-ylcarbonyloxy)phenylalanine
N-(toluene-4-sulfonyl)-L-prolyl-L-3-(N,N-dimethylcarbamyloxy)phenylalanine ethyl ester
N-(toluene-4-sulfonyl)-L-prolyl-L-4-(1-tert-butylcarbonyloxy-4-phenylpiperidin-4-ylcarbonyloxy)phenylalanine ethyl ester
N-(toluene-4-sulfonyl)-L-(5,5-dimethyl)thiaprolyl-L-4-(N,N-dimethylcarbamyloxy)phenylalanine isopropyl ester
N-(toluene-4-sulfonyl)-L-(5,5-dimethyl)thiaprolyl-L-4-(N,N-dimethylcarbamyloxy)phenylalanine tert-butyl ester
N-(toluene-4-sulfonyl)-L-(5,5-dimethyl)thiaprolyl-L-4-(N,N-dimethylcarbamyloxy)phenylalanine
N-(toluene-4-sulfonyl)-L-[(1,1-dioxo)thiamorpholin-3-carbonyl]-L-4-(N,N-dimethylcarbamyloxy)phenylalanine tert-butyl ester
N-(toluene-4-sulfonyl)-L-[(1,1-dioxo)thiamorpholin-3-carbonyl]-L-4-(N,N-dimethylcarbamyloxy)phenylalanine
N-(toluene-4-sulfonyl)-L-(5,5-dimethyl)thiaprolyl-L-4-(4-methylpiperazin-1-ylcarbonyloxy)phenylalanine tert-butyl ester
N-(toluene-4-sulfonyl)-L-(5,5-dimethyl)thiaprolyl-L-4-(4-methylpiperazin-1-ylcarbonyloxy)phenylalanine
N-(1-methylimidazole-4-sulfonyl)-L-prolyl-L-4-(N,N-dimethylcarbamyloxy)phenylalanine tert-butyl ester
N-(4-aminobenzenesulfonyl)-L-prolyl-L-4-(N,N-dimethylcarbamyloxy)phenylalanine tert-butyl ester
N-(toluene-4-sulfonyl)-L-prolyl-L-4-(morpholin-4-ylcarbonyloxy)phenylalanine tert-butyl ester
N-(α-toluenesulfonyl)-L-(5,5-dimethyl)thiaprolyl-L-4-(N,N-dimethylcarbamyloxy)phenylalanine
N-(toluene-4-sulfonyl)-L-(piperazin-2-carbonyl)-L-4-(N,N-dimethylcarbanyloxy)phenylalanine
N-(α-toluenesulfonyl)-L-(5,5-dimethyl)thiaprolyl-L-4-(N,N-dimethylcarbarnyloxy)phenylalanine tert-butyl ester
N-(toluene-4-sulfonyl)-L-(piperazin-2-carbonyl)-L-4-(N,N-dimethylcarbamyloxy)phenylalanine tert-butyl ester
N-(toluene-4-sulfonyl)-L-(4-benzyloxycarbonylpiperazin-2-carbonyl)-L-4-(N,N-dimethylcarbamyloxy)phenylalanine tert-butyl ester
N-(toluene-4-sulfonyl)-L-[(1,1-dioxo)thiamorpholin-3-carbonyl]-L-4-(morpholin-4-ylcarbonyloxy)phenylalanine tert-butyl ester
N-(toluene-4-sulfonyl)-L-[(1,1-dioxo)thiamorpholin-3-carbonyl]-L-4-(morpholin-4-ylcarbonyloxy)phenylalanine
N-(1-methylpyrazole-4-sulfonyl)-L-prolyl-L-4-(N,N-dimethylcarbamyloxy)phenylalanine tert-butyl ester
N-(4-fluorobenzenesulfonyl)-L-prolyl-L-4-(N,N-dimethylcarbanyloxy)phenylalanine tert-butyl ester
N-(toluene-4-sulfonyl)-L-(1,1-dioxo-5,5-dimethyl)thiaprolyl-L-4-(N,N-dimethylcarbarnyloxy)phenylalanine tert-butyl ester
N-(1-methylimidazole-4-sulfonyl)-L-prolyl-L-4-(N,N-dimethylcarbamyloxy)phenylalanine
N-(toluene-4-sulfonyl)-L-(1,1-dioxo-5,5-dimethyl)thiaprolyl-L-4-(N,N-dimethylcarbamyloxy)phenylalanine
N-(4-fluorobenzenesulfonyl)-L-prolyl-L-4-(N,N-dimethylcarbamyloxy)phenylalanine
N-(pyridine-3-sulfonyl)-L-prolyl-L-4-(N,N-dimethylcarbamyloxy)phenylalanine
N-(toluene-4-sulfonyl)-D-prolyl-L-4-(4-methylpiperazin-1-ylcarbonyloxy)phenylalanine tert-butyl ester
N-(4-nitrobenzenesulfonyl)-L-prolyl-L-4-(N,N-dimethylcarbamyloxy)phenylalanine tert-butyl ester
N-(toluene-4-sulfonyl)-L-prolyl-L-4-(1,1-dioxothiomorpholin-4-ylcarbonyloxy)phenylalanine
N-(toluene-4-sulfonyl)-L-prolyl-L-4-(thiomorpholin-4-ylcarbonyloxy)phenylalanine
N-(toluene-4-sulfonyl)-L-prolyl-L-4-(pyrrolidin-1-ylcarbonyloxy)phenylalanine
N-(toluene-4-sulfonyl)-L-prolyl-L-4-(morpholin-4-ylcarbonyloxy)phenylalanine
N-(toluene-4-sulfonyl)-L-prolyl-L-4-(4-methylpiperazin-1-ylcarbonyloxy)phenylalanine neopentyl ester
N-(toluene-4-sulfonyl)-L-prolyl-L-4-(N,N-dimethylcarbamyloxy)phenylalanine neopentyl ester
N-(toluene-4-sulfonyl)-L-prolyl-L-4-(4-tert-butyloxycarbonylpiperazin-1-ylcarbonyloxy)phenylalanine ethyl ester
N-(toluene-4-sulfonyl)-L-prolyl-L-4-(morpholin-4-ylcarbonyloxy)phenylalanine ethyl ester
N-(toluene-4-sulfonyl)-L-(thiamorpholin-3-carbonyl)-L-4-(N,N-dimethylcarbamyloxy)phenylalanine tert-butyl ester
N-(toluene-4-sulfonyl)-L-(1,1-dioxothiamorpholin-3-carbonyl)-L-4-(N,N-dimethylcarbamyloxy)phenylalanine tert-butyl ester
N-(toluene-4-sulfonyl)-L-(1,1-dioxothiamorpholin-3-carbonyl)-L-4-(morpholin-4-ylcarbonyloxy)phenylalanine tert-butyl ester
N-(4-fluorobenzenesulfonyl)-L-(thiamorpholin-3-carbonyl)-L-4-(N,N-dimethylcarbamyloxy)phenylalanine tert-butyl ester
N-(4-fluorobenzenesulfonyl)-L-(1,1-dioxothiamorpholin-3-carbonyl)-L-4-(N,N-dimethylcarbamyloxy)phenylalanine tert-butyl ester
N-(pyridine-3-sulfonyl)-L-prolyl-L-4-(N,N-dimethylcarbamyloxy)phenylalanine tert-butyl ester
N-(pyrimidine-2-sulfonyl)-L-prolyl-L-4-(N,N-dimethylcarbamyloxy)phenylalanine tert-butyl ester
N-(4-nitrobenzenesulfonyl)-L-prolyl-L-4-(N,N-dimethylcarbamyloxy)phenylalanine
N-(4-cyanobenzenesulfonyl)-L-prolyl-L-4-(N,N-dimethylcarbamyloxy)phenylalanine tert-butyl ester
N-(toluene-4-sulfonyl)-L-(1,1-dioxothiamorpholin-3-carbonyl)-L-4-(N,N-dimethylcarbamyloxy)phenylalanine
N-(toluene-4-sulfonyl)-L-(1,1-dioxo)thiaprolyl-L-4-(N,N-dimethylcarbamyloxy)phenylalanine tert-butyl ester
N-(4-fluorobenzenesulfonyl)-L-prolyl-L-4-(N,N-dimethylcarbamyloxy)phenylalanine tert-butyl ester
N-(1-methylpyrazole-4-sulfonyl)-L-prolyl-L-4-(N,N-dimethylcarbamyloxy)phenylalanine
N-(toluene-4-sulfonyl)-L-(1,1-dioxo)thiaprolyl-L-4-(N,N-dimethylcarbamyloxy)phenylalanine
N-(4-fluorobenzenesulfonyl)-L-thiaprolyl-L-4-(N,N-dimethylcarbamyloxy)phenylalanine
N-(toluene-4-sulfonyl)-L-prolyl-L-4-(piperazin-1-ylcarbonyloxy)phenylalanine
N-(toluene-4-sulfonyl)-L-prolyl-L-4-(1-tert-butyloxycarbonylpiperazin-1-ylcarbonyloxy)phenylalanine tert-butyl ester
N-(toluene-4-sulfonyl)-L-prolyl-L-4-(piperazin-1-ylcarbonyloxy)phenylalanine ethyl ester
N-(toluene-4-sulfonyl)-L-prolyl-L-4-(4-acetylpiperazin-1-ylcarbonyloxy)phenylalanine ethyl ester
N-(toluene-4-sulfonyl)-L-prolyl-L-4-(4-methanesulfonylpiperazin-1-ylcarbonyloxy)phenylalanine ethyl ester
N-(toluene-4-sulfonyl)-L-prolyl-L-4-(morpholin-4-ylcarbonyloxy)-3-nitrophenylalanine
N-(toluene-4-sulfonyl)-L-prolyl-L-4-(1-tert-butyloxycarbonylpiperazin-1-ylcarbonyloxy)phenylalanine
N-(4-fluorobenzenesulfonyl)-L-(5,5-dimethyl)thiaprolyl-L-4-(N,N-dimethylcarbamyloxy)phenylalanine tert-butyl ester
N-(4-fluorobenzenesulfonyl)-L-(1,1-dioxothiamorpholin-3-carbonyl)-L-4-(1,1-dioxothiomorpholin-4-ylcarbonyloxy)phenylalanine tert-butyl ester
N-(toluene-4-sulfonyl)-L-(1,1-dioxothiamorpholin-3-carbonyl)-L-4-(1,1-dioxothiomorpholin-4-ylcarbonyloxy)phenylalanine tert-butyl ester
N-(4-fluorobenzenesulfonyl)-L-prolyl-L-4-(1,1-dioxothiomorpholin-4-ylcarbonyloxy)phenylalanine tert-butyl ester
N-(4-fluorobenzenesulfonyl)-L-prolyl-L-4-(thiomorpholin-4-ylcarbonyloxy)phenylalanine tert-butyl ester
N-(4-fluorobenzenesulfonyl)-L-prolyl-L-4-(morpholin-4-ylcarbonyloxy)phenylalanine tert-butyl ester
N-(4-fluorobenzenesulfonyl)-L-(5,5-dimethyl)thiaprolyl-L-4-(N,N-dimethylcarbamyloxy)phenylalanine
N-(4-fluorobenzenesulfonyl)-L-(1,1-dioxothiamorpholin-3-carbonyl)-L-4-(morpholin-4-ylcarbonyloxy)phenylalanine tert-butyl ester
N-(4-trifluoromethoxybenzenesulfonyl)-L-prolyl-L-4-(N,N-dimethylcarbamyloxy)phenylalanine tert-butyl ester
N-(toluene-4-sulfonyl)-L-(1,1-dioxothiamorpholin-3-carbonyl)-L-4-(N,N-dimethylcarbamyloxy)phenylalanine isopropyl ester
N-(4-fluorobenzenesulfonyl)-L-(1,1-dioxo-5,5-dimethyl)thiaprolyl-L-4-(N,N-dimethylcarbamyloxy)phenylalanine tert-butyl ester
N-(toluene-4-sulfonyl)-L-(1,1-dioxo-5,5-dimethyl)thiaprolyl-L-4-(N,N-dimethylcarbamyloxy)phenylalanine isopropyl ester
N-(toluene-4-sulfonyl)-L-(1,1-dioxothiamorpholin-3-carbonyl)-L-4-(morpholin-4-ylcarbonyloxy)phenylalanine
N-(4-fluorobenzenesulfonyl)-L-(5,5-dimethyl)thiaprolyl-L-4-(N,N-dimethylcarbamyloxy)phenylalanine
N-(toluene-4-sulfonyl)-L-(1,1-dioxothiamorpholin-3-carbonyl)-L-4-(4-methylpiperazin-1-ylcarbonyloxy)phenylalanine tert-butyl ester
N-(1-methylpyrazole-4-sulfonyl)-L-prolyl-L-4-(4-methylpiperazin-1-ylcarbonyloxy)phenylalanine tert-butyl ester
N-(4-fluorobenzenesulfonyl)-L-(1,1-dioxo)thiaprolyl-L-4-(N,N-dimethylcarbamyloxy)phenylalanine
N-(toluene-4-sulfonyl)-L-prolyl-L-4-(1,1-dioxothiomorpholin-4-ylcarbonyloxy)phenylalanine tert-butyl ester
N-(toluene-4-sulfonyl)-L-prolyl-L-4-(thiomorpholin-4-ylcarbonyloxy)phenylalanine tert-butyl ester
N-(toluene-4-sulfonyl)-L-prolyl-L-4-(pyrrolidin-1-ylcarbonyloxy)phenylalanine tert-butyl ester
N-(4-fluorobenzenesulfonyl)-L-thiaprolyl-L-4-(N,N-dimethylcarbamyloxy)phenylalanine tert-butyl ester
N-(4-fluorobenzenesulfonyl)-L-(1,1-dioxo)thiaprolyl-L-4-(N,N-dimethylcarbamyloxy)phenylalanine tert-butyl ester
N-(2,5-dichlorothiophene-3-sulfonyl)-L-prolyl-L-4-(N,N-dimethylcarbamyloxy)phenylalanine tert-butyl ester
N-(4-acetamidobenzenesulfonyl)-L-prolyl-L-4-(N,N-dimethylcarbamyloxy)phenylalanine tert-butyl ester
N-(4-tert-butylbenzenesulfonyl)-L-(1,1-dioxothiamorpholin-3-carbonyl)-L-4-(N,N-dimethylcarbamyloxy)phenylalanine tert-butyl ester
N-(pyridine-2-sulfonyl)-L-prolyl-L-4-(N,N-dimethylcarbamyloxy)phenylalanine
N-(2-fluorobenzenesulfonyl)-L-(1,1-dioxothiamorpholin-3-carbonyl)-L-4-(N,N-dimethylcarbamyloxy)phenylalanine tert-butyl ester
N-(3-fluorobenzenesulfonyl)-L-(1,1-dioxothiamorpholin-3-carbonyl)-L-4-(N,N-dimethylcarbamyloxy)phenylalanine tert-butyl ester
N-(2,4-difluorobenzenesulfonyl)-L-(1,1-dioxothiamorpholin-3-carbonyl)-L-4-(N,N-dimethylcarbamyloxy)phenylalanine tert-butyl ester
N-(4-acetamidobenzenesulfonyl)-L-prolyl-L-4-(N,N-dimethylcarbamyloxy)phenylalanine
N-(4-trifluoromethoxybenzenesulfonyl)-L-prolyl-L-4-(N,N-dimethylcarbamyloxy)phenylalanine
N-(4-fluorobenzenesulfonyl)-L-(5,5-dimethyl)thiaprolyl-L-4-(N,N-dimethylcarbamyloxy)phenylalanine tert-butyl ester
N-(4-cyanobenzenesulfonyl)-L-prolyl-L-4-(N,N-dimethylcarbamyloxy)phenylalanine
N-(toluene-4-sulfonyl)-L-(3,3-dimethyl)prolyl-L-4-(N,N-dimethylcarbamyloxy)phenylalanine tert-butyl ester
N-(toluene-4-sulfonyl)-L-(3,3-dimethyl)prolyl-L-4-(N,N-dimethylcarbamyloxy)phenylalanine isopropyl ester
N-(1-methylpyrazole-4-sulfonyl)-L-prolyl-L-4-(N,N-dimethylcarbamyloxy)phenylalanine iso-propyl ester
N-(1-methylpyrazole-4-sulfonyl)-L-(5,5-dimethyl)thiaprolyl-L-4-(N,N-dimethylcarbamyloxy)phenylalanine tert-butyl ester
N-(1-methylpyrazole-4-sulfonyl)-L-(5,5-dimethyl)thiaprolyl-L-4-(N,N-dimethylcarbamyloxy)phenylalanine
N-(toluene-4-sulfonyl)-L-prolyl-L-4-(4′-acetylpiperazin-1-ylcarbonyloxy)phenylalanine
N-(toluene-4-sulfonyl)-L-prolyl-L-4-(4′-methanesulfonylpiperazin-1-ylcarbonyloxy)phenylalanine
N-(toluene-4-sulfonyl)-L-prolyl-L-4-(4′-phenylpiperazin-1-ylcarbonyloxy)phenylalanine
N-(toluene-4-sulfonyl)-L-prolyl-L-4-(piperazin-1-ylcarbonyloxy)phenylalanine tert-butyl ester
N-(toluene-4-sulfonyl)-L-prolyl-L-4-(4′-methanesulfonylpiperazin-1-ylcarbonyloxy)phenylalanine tert-butyl ester
N-(toluene-4-sulfonyl)-L-prolyl-L-4-(N,N-dimethylcarbamyloxy)phenylalanine (N-tert-butoxycarbonyl-2-amino-2-methylpropyl) ester
N-(toluene-4-sulfonyl)-L-prolyl-L-4-(4′-acetylpiperazin-1-ylcarbonyloxy)phenylalanine tert-butyl ester
N-(toluene-4-sulfonyl)-L-prolyl-L-4-(4-hydroxypiperidin-1-ylcarbonyloxy)phenylalanine tert-butyl ester
N-(toluene-4-sulfonyl)-L-prolyl-L-4-(N-(2-(morpholin-4′-yl)ethyl)carbamyloxy)phenylalanine tert-butyl ester
N-(toluene-4-sulfonyl)-L-prolyl-L-4-(N-(2-hydroxyethyl)-N-methylcarbamyloxy)phenylalanine tert-butyl ester
N-(toluene-4-sulfonyl)-L-prolyl-4-(4′-(2-hydroxyethyl)piperazin-1-ylcarbonyloxy)-L-phenylalanine tert-butyl ester
N-(toluene-4-sulfonyl)-L-prolyl-L-4-(N-(2-formyloxyethyl)-N-methylcarbamyloxy)phenylalanine
N-(toluene-4-sulfonyl)-L-prolyl-L-4-(N-(2′-hydroxyethyl)-N-methylcarbamyloxy)phenylalanine isopropyl ester
N-(toulene-4-sulfonyl)-L-prolyl-L-4-(N-(methoxycarbonylmethyl)carbamyloxy)phenylalanine tert-butyl ester
N-(1-methylpyrazole-4-sulfonyl)-L-(5,5-dimethyl)thiaprolyl-L-(4-N,N-dimethylcarbamyloxy)phenylalanine isopropyl ester
N-(toluene-4-sulfonyl)-L-prolyl-L-4-(4′-methoxypiperidin-1-ylcarbonyloxy)phenylalanine isopropyl ester
N-(toluene-4-sulfonyl)-L-prolyl-L-4-(4′-methoxypiperidin-1-ylcarbonyloxy)phenylalanine
N-(toluene-4-sulfonyl)-L-4-oxoprolyl-L-4-(N,N-dimethylcarbamyloxy)phenylalanine tert-butyl ester
N-(toluene-4-sulfonyl)-L-trans-4-hydroxyprolyl-L-4-(N,N-dimethylcarbamyloxy)phenylalanine tert-butyl ester
N-(3-fluorobenzenesulfonyl)-L-prolyl-L-4-(N,N-dimethylcarbamyloxy)phenylalanine tert-butyl ester
N-(morpholino-sulfonyl)-L-prolyl-L-(4-N,N-dimethylcarbamyloxy)phenylalanine tert-butyl ester
N-(morpholino-sulfonyl)-L-prolyl-L-(4-N,N-dimethylcarbamyloxy)phenylalanine
N-(1-methylpyrazole-4-sulfonyl)-L-(1,1-dioxothiamorpholin-3-carbonyl)-L-4-(N,N-dimethylcarbamyloxy)phenylalanine tert-butyl ester
N-(2-fluorobenzenesulfonyl)-L-(1,1-dioxothiamorpholin-3-carbonyl)-L-4-(N,N-dimethylcarbamyloxy)phenylalanine
N-(2,4-difluorobenzenesulfonyl)-L-(1,1-dioxothiamorpholin-3-carbonyl)-L-4-(N,N-dimethylcarbamyloxy)phenylalanine
N-(toluene-4-sulfonyl)-L-(thiamorpholin-3-carbonyl)-L-4-(N,N-dimethylcarbamyloxy)phenylalanine
N-(pyridine-3-sulfonyl)-L-(5,5-dimethyl-thiaprolyl-L-4-(N,N-dimethylcarbamyloxy)phenylalanine isopropyl ester
N-(3-fluorobenzenesulfonyl)-L-(1,1-dioxothiamorpholin-3-carbonyl)-L-4-(N,N-dimethylcarbamyloxy)phenylalanine
N-(1-methylpyrazole-4-sulfonyl)-L-(1,1-dioxothiamorpholin-3-carbonyl)-L-4-(N,N-dimethylcarbamyloxy)phenylalanine
N-(4-tert-butylbenzenesulfonyl)-L-(1,1-dioxothiamorpholin-3-carbonyl)-L-4-(N,N-dimethylcarbarnyloxy)phenylalanine
N-(toluene-4-sulfonyl)-(3,3-dimethyl)prolyl-L-4-(N,N-dimethylcarbamyloxy)phenylalanine
N-(2,5-dichlorothiophene-3-sulfonyl)-L-prolyl-L-4-(N,N-dimethylcarbamyloxy)phenylalanine
N-(4-methoxybenzenesulfonyl)-L-prolyl-L-4-(N,N-dimethylcarbamyloxy)phenylalanine
N-(4-methoxybenzenesulfonyl)-L-prolyl-L-4-(N,N-dimethylcarbamyloxy)phenylalanine isopropyl ester
N-(toluene-4-sulfonyl)-L-(1-oxo-thiomorpholin-3-carbonyl)-L-4-(N,N-dimethylcarbamyloxy)phenylalanine
N-(toluene-4-sulfonyl)-L-(1-oxo-thiomorpholin-3-carbonyl)-L-4-(N,N-dimethylcarbamyloxy)phenylalanine tert-butyl ester
N-(3,4-difluorobenzenesulfonyl)-L-prolyl-4-(N,N-dimethylcarbamyloxy)phenylalanine isopropyl ester
N-(3,4-difluorobenzenesulfonyl)-L-prolyl-4-(N,N-dimethylcarbamyloxy)phenylalanine
N-(3,4-difluorobenzenesulfonyl)-L-(1,1-dioxothiamorpholin-3-carbonyl)-L-4-(N,N-dimethylcarbamyloxy)phenylalanine tert-butyl ester
N-(3,4-difluorobenzenesulfonyl)-L-(1,1-dioxothiamorpholin-3-carbonyl)-L-4-(N,N-dimethylcarbarnyloxy)phenylalanine
N-(toluene-4-sulfonyl)-L-(5,5-dimethyl)thiaprolyl-L-(thiomorpholin-4-ylcarbonyloxy)phenylalanine tert-butyl ester
N-(toluene-4-sulfonyl)-L-(5,5-dimethyl)thiaprolyl-L-(thiomorpholin-4-ylcarbonyloxy)phenylalanine
N-(1-methylpyrazole-4-sulfonyl)-L-(5,5-dimethyl)thiaprolyl-L-4-(N,N-dimethylcarbarnyloxy)phenylalanine ethyl ester
N-(pyridine-3-sulfonyl)-L-(5,5-dimethyl)thiaprolyl-L-4-(N,N-dimethylcarbamyloxy)phenylalanine
N-(pyridine-2-sulfonyl)-L-prolyl-L-4-(N,N-dimethylcarbamyloxy)phenylalanine isopropyl ester
N-(pyridine-2-sulfonyl)-L-prolyl-L-4-(N,N-dimethylcarbarnyloxy)phenylalanine
N-(pyridine-2-sulfonyl)-L-(5,5-dimethyl)thiaprolyl-L-4-(N,N-dimethylcarbamyloxy)phenylalanine isopropyl ester
N-(pyridine-2-sulfonyl)-L-(5,5-dimethyl)thiaprolyl-L-4-(N,N-dimethylcarbamyloxy)phenylalanine
N-(toluene-4-sulfonyl)-L-(thiamorpholin-3-carbonyl)-L-4-(N,N-dimethylcarbamyloxy)phenylalanine isopropyl ester
N-(3-fluorobenzenesulfonyl)-L-(5,5-dimethyl)thiaprolyl-L-4-(N,N-dimethylcarbamyloxy)phenylalanine isopropyl ester
N-(2-fluorobenzenesulfonyl)-L-(5,5-dimethyl)thiaprolyl-L-4-(N,N-dimethylcarbamyloxy)phenylalanine isopropyl ester
N-(3,4-difluorobenzenesulfonyl)-L-(5,5-dimethyl)thiaprolyl-L-4-(N,N-dimethylcarbamyloxy)phenylalanine isopropyl ester
N-(3,5-difluorobenzenesulfonyl)-L-(5,5-dimethyl)thiaprolyl-L-4-(N,N-dimethylcarbamyloxy)phenylalanine isopropyl ester
N-(2,4-difluorobenzenesulfonyl)-L-(5,5-dimethyl)thiaprolyl-L-4-(N,N-dimethylcarbamyloxy)phenylalanine isopropyl ester
N-(4-chlorobenzenesulfonyl)-L-(5,5-dimethyl)thiaprolyl-L-4-(N,N-dimethylcarbamyloxy)phenylalanine isopropyl ester
N-(3-chlorobenzenesulfonyl)-L-(5,5-dimethyl)thiaprolyl-L-4-(N,N-dimethylcarbamyloxy)phenylalanine isopropyl ester
N-(2-chlorobenzenesulfonyl)-L-(5,5-dimethyl)thiaprolyl-L-4-(N,N-dimethylcarbamyloxy)phenylalanine isopropyl ester
N-(3,4-dichlorobenzenesulfonyl)-L-(5,5-dimethyl)thiaprolyl-L-4-(N,N-dimethylcarbamyloxy)phenylalanine isopropyl ester
N-(3,5-dichlorobenzenesulfonyl)-L-(5,5-dimethyl)thiaprolyl-L-4-(N,N-dimethylcarbamyloxy)phenylalanine isopropyl ester
N-(3-chlorobenzenesulfonyl)-L-(1,1-dioxothiamorpholin-3-carbonyl)-L-4-(N,N-dimethylcarbamyloxy)phenylalanine tert-butyl ester
N-(3,4-dichlorobenzenesulfonyl)-L-(1,1-dioxothiamorpholin-3-carbonyl)-L-4-(N,N-dimethylcarbamyloxy)phenylalanine tert-butyl ester
N-(4-methoxybenzenesulfonyl)-L-(5,5-dimethyl)thiaprolyl-L-4-(N,N-dimethylcarbamyloxy)phenylalanine isopropyl ester
N-(3-methoxybenzenesulfonyl)-L-(5,5-dimethyl)thiaprolyl-L-4-(N,N-dimethylcarbamyloxy)phenylalanine isopropyl ester
N-(2-methoxybenzenesulfonyl)-L-(5,5-dimethyl)thiaprolyl-L-4-(N,N-dimethylcarbamyloxy)phenylalanine isopropyl ester
N-(3,4-dimethoxybenzenesulfonyl)-L-(5,5-dimethyl)thiaprolyl-L-4-(N,N-dimethylcarbamyloxy)phenylalanine isopropyl ester
N-(2,4-difluorobenzenesulfonyl)-L-(thiamorpholin-3-carbonyl)-L-4-(N,N-dimethylcarbamyloxy)phenylalanine isopropyl ester
N-(3,4-dichlorobenzenesulfonyl)-L-(1,1-dioxothiamorpholin-3-carbonyl)-L-4-(N,N-dimethylcarbamyloxy)phenylalanine
N-(3-chlorobenzenesulfonyl)-L-(1,1-dioxothiamorpholin-3-carbonyl)-L-4-(N,N-dimethylcarbamyloxy)phenylalanine
N-(3-chloro-4-fluorobenzenesulfonyl)-L-(1,1-dioxothiamorpholin-3-carbonyl)-L-4-(N,N-dimethylcarbarnyloxy)phenylalanine tert-butyl ester
N-(1-methylpyrazole-4-sulfonyl)-L-(thiamorpholin-3-carbonyl)-L-4-(N,N-dimethylcarbamyloxy)phenylalanine tert-butyl ester
N-(3,4-difluorobenzenesulfonyl)-L-(thiamorpholin-3-carbonyl)-L-4-(N,N-dimethylcarbamyloxy)phenylalanine tert-butyl ester
N-(toluene-4-sulfonyl)-L-(5,5-dimethyl)thioprolyl-L-(thiomorpholin-4-ylcarbonyloxy)phenylalanine isopropyl ester
N-(3,4-difluorobenzenesulfonyl)-L-(thiamorpholin-3-carbonyl)-L-4-(N,N-dimethylcarbamyloxy)phenylalanine
N-(2,5-dichlorothiophene-3-sulfonyl)-L-(5,5-dimethyl)thiaprolyl-L-4-(N,N-dimethylcarbamyloxy)phenylalanine isopropyl ester
N-(1-methylpyrazole-4-sulfonyl)-L-(5,5-dimethyl)thiaprolyl-L-4-(thiomorpholin-4-ylcarbonyloxy)phenylalanine isopropyl ester
N-(8-quinolinesulfonyl)-L-prolyl-L-4-(N,N-dimethylcarbamyloxy)phenylalanine isopropyl ester
N-(8-quinolinesulfonyl)-L-prolyl-L-4-(N,N-dimethylcarbamyloxy)phenylalanine
N-(8-quinolinesulfonyl)-L-(5,5-dimethyl)thiaprolyl-L-4-(N,N-dimethylcarbamyloxy)phenylalanine isoproplyl ester
N-(8-quinolinesulfonyl)-L-(5,5-dimethyl)thiaprolyl-L-4-(N,N-dimethylcarbamyloxy)phenylalanine
N-(toluene-4-sulfonyl)-L-prolyl-L-4-(4-phenylpiperazin-1-ylcarbonyloxy)phenylalanine tert-butyl ester
N-(toluene-4-sulfonyl)-L-prolyl-L-4-(4′-(ethoxycarbonyl)piperidin-1-ylcarbonyloxy)phenylalanine tert-butyl ester
N-(pyridine-3-sulfonyl)-L-(5,5-dimethyl)thiaprolyl-L-4-(N,N-dimethylcarbamyloxy)phenylalanine tert-butyl ester
N-(3-sulfonamido-4-chloro-benzenesulfonyl)-L-prolyl-L-4-(N,N-dimethylcarbanyloxy)phenylalanine isopropyl ester
N-(toluene-4-sulfonyl)-L-(1-oxothiomorpholin-3-carbonyl)-L-4-(N,N-dimethylcarbamyloxy)phenylalanine isopropyl ester
N-(2,4-difluorobenzenefulfonyl)-L-(1-oxothiomorpholin-3-carbonyl)-L-4-(N,N-dimethylcarbamyloxy)phenylalanine tert-butyl ester
N-(1-methylpyrazole-4-sulfonyl)-L-(5,5-dimethyl)thiaprolyl-L-4-(N,N-dimethylcarbamyloxy)phenylalanine 2,2-dimethylpropyl ester
N-(pyridine-3-sulfonyl)-L-(5,5-dimethyl)thiaprolyl-L-4-(N,N-dimethylcarbamyloxy)phenylalanine 2,2-dimethylpropyl ester
N-(1-methylpyrazole-4-sulfonyl)-L-(5,5-dimethyl)thiaprolyl-L-4-(N,N-dimethylcarbamyloxy)phenylalanine cyclopropylmethyl ester
N-(1-methylpyrazole-4-sulfonyl)-L-(5,5-dimethyl)thiaprolyl-L-4-(N,N-dimethylcarbamyloxy)phenylalanine methyl ester
N-(pyridine-3-sulfonyl)-L-(5,5-dimethyl)thiaprolyl-L-4-(N,N-dimethylcarbamyloxy)phenylalanine ethyl ester
N-(pyridine-3-sulfonyl)-L-(5,5-dimethyl)thiaprolyl-L-4-(N,N-dimethylcarbamyloxy)phenylalanine cyclopropylmethyl ester
N-(1-methylpyrazole-4-sulfonyl)-L-(5,5-dimethyl)thiaprolyl-L-4-(N,N-dimethylcarbamyloxy)phenylalanine 2-methoxyphenyl ester
N-(1-methylpyrazole-4-sulfonyl)-L-(5,5-dimethyl)thiaprolyl-L-4-(N,N-dimethylcarbamyloxy)phenylalanine n-butyl ester
N-(1-methylpyrazole-4-sulfonyl)-L-(5,5-dimethyl)thiaprolyl-L-4-(N,N-dimethylcarbamyloxy)phenylalanine n-propyl ester
N-(1-methylpyrazole-4-sulfonyl)-L-(5,5-dimethyl)thiaprolyl-L-4-(N,N-dimethylcarbamyloxy)phenylalanine 2,2-dimethylpropionyloxymethyl ester
N-(toluene-4-sulfonyl)-L-prolyl-L-4-(N-(4′-(2′-aminoethyl)morpholino)carbamyloxy)phenylalanine
N-(toluene-4-sulfonyl)-L-prolyl-L-4-[4-(carboxy)piperidin 1-ylcarbonyloxy]phenylalanine
N-(toluene-4-sulfonyl)-L-prolyl-L-4-(N,N-bis-(2-hydroxyethyl)carbamyloxy)phenylalanine isopropyl ester
N-(toluene-4-sulfonyl)-L-prolyl-L-4-[3-(hydroxymethyl)piperidin-1-ylcarbonyloxy]phenylalanine isopropyl ester
N-(toluene-4-sulfonyl)-L-prolyl-L-4-(4-trifluoromethanesulfonylpiperazin-1-ylcarbonyloxy)phenylalanine tert-butyl ester
N-(4-(N-phenylurea)benzenesulfonyl)-L-prolyl-L-4-(N,N-dimethylcarbamyloxy)phenylalanine tert-butyl ester
N-(2-trifluoroacetyl-1,2,3,4-tetrahydroisoquinolin-7-sulfonyl)-L-prolyl-L-4-(N,N-dimethylcarbamyloxy)phenylalanine isopropyl ester
N-(1-methylpyrazole-3-sulfonyl)-L-(5,5-dimethyl)thiaprolyl-L-4-(N,N-dimethylcarbamyloxy)phenylalanine isopropyl ester
N-(1-methylpyrazole-3-sulfonyl)-L-(5,5-dimethyl)thiaprolyl-L-4-(N,N-dimethylcarbamyloxy)phenylalanine
N-(pyridine-4-sulfonyl)-L-(5,5-dimethyl)thiaprolyl-L-4-(N,N-dimethylcarbamyloxy)phenylalanine isopropyl ester
N-(pyridine-4-sulfonyl)-L-(5,5-dimethyl)thiaprolyl-L-4-(N,N-dimethylcarbamyloxy)phenylalanine
N-(toluene-4-sulfonyl)-L-(5,5-dimethyl)thiaprolyl-L-4-(N-methyl-N-(2-dimethylaminoethyl)carbamyloxy)phenylalanine tert-butyl ester
N-(toluene-4-sulfonyl)-L-prolyl-L-4-(N-methyl-N-(2-dimethylaminoethyl)carbamyloxy)phenylalanine tert-butyl ester
N-(toluene-4-sulfonyl)-L-(5,5-dimethyl)thiapropyl-L-4-(N-methyl-N-(2-dimethylaminoethyl)carbamyloxy)phenylalanine
N-(toluene-4-sulfonyl)-L-prolyl-L-4-(N-methyl-N-(2-dimethylaminoethyl)carbamyloxy)phenylalanine
N-(4-fluorobenzenesulfonyl)-L-(5,5-dimethyl)thiaprolyl-L-3-chloro-4-(4-methylpiperazin-1-ylcarbonyloxy)phenylalanine tert-butyl ester
N-(4-fluorobenzenesulfonyl)-L-(5,5-dimethyl)thiaprolyl-L-3-chloro-4-(N,N-dimethycarbamyloxy)phenylalanine
N-(4-fluorobenzenesulfonyl)-L-(5,5-dimethyl)thiaprolyl-L-3-chloro-4-(thiomorpholin-4-ylcarbonyloxy)phenylalanine tert-butyl ester
N-(4-fluorobenzenesulfonyl)-L-(5,5-dimethyl)thiaprolyl-L-3-chloro-4-(thiomorpholin-4-ylcarbonyloxy)phenylalanine isopropyl ester
N-(4-fluorobenzenesulfonyl)-L-(5,5-dimethyl)thiaprolyl-L-3-chloro-4-(N,N-dimethylcarbamyloxy)]phenylalanine isopropyl ester
N-(toluene-4-sulfonyl)-L-(5,5-dimethyl)thiaprolyl-L-3-chloro-4-(N,N-dimethylcarbamyloxy)phenylalanine isopropyl ester
N-(4-fluorobenzenesulfonyl)-L-(5,5-dimethyl)thiaprolyl-L-3-chloro-4-(4-methylpiperazin-1-ylcarbonyloxy)]phenylalanine isopropyl ester
N-(toluene-4-sulfonyl)-L-prolyl-L-3-chloro-4-(N,N-dimethylcarbamyloxy)]phenylalanine isopropyl ester
N-(4-fluorobenzenesulfonyl)-L-(5,5-dimethyl)thiaprolyl-L-3-chloro-4-(4-(2′-pyridyl)-piperazin-1-ylcarbonyloxy)]phenylalanine isopropyl ester
N-(4-fluorobenzenesulfonyl)-L-(5,5-dimethyl)thiaprolyl-L-3-chloro-4-(4-(2′-pyridyl)-piperazin-1-ylcarbonyloxy)]phenylalanine tert-butyl ester
N-(4-nitrobenzenesulfonyl)-L-prolyl-L-4-(N,N-dimethylcarbamyloxy)phenylalanine isopropyl ester
N-(4-aminobenzenesulfonyl)-L-prolyl-L-4-(N,N-dimethylcarbamyloxy)phenylalanine isopropyl ester
N-(toluene-4-sulfonyl)-L-prolyl-L-4-(thiomorpholin-4-ylcarbonyloxy)phenylalanine isopropyl ester
N-(toluene-4-sulfonyl)-L-prolyl-L-4-(4-phenylcarbamylpiperazin-1-ylcarbonyloxy)phenylalanine isopropyl ester
N-(toluene-4-sulfonyl)-L-prolyl-L-4-(4-phenylcarbamylpiperazin-1-ylcarbonyloxy)phenylalanine
N-(1-n-butylpyrazole-4-sulfonyl)-L-(5,5-dimethyl)thiaprolyl-L-4-(N,N-dimethylcarbamyloxy)phenylalanine isopropyl ester
N-(toluene-4-sulfonyl)-L-prolyl-L-4-(pyridin-4-ylcarbonyl)piperazin-1-ylcarbonyloxy)phenylalanine isopropyl ester
N-(toluene-4-sulfonyl)-L-4-oxoprolyl-L-4-(N,N-dimethylcarbamyloxy)phenylalanine
N-(toluene-4-sulfonyl)-L-trans-4-hydroxyprolyl-L-4-(N,N-dimethylcarbamyloxy)phenylalanine
N-(4-cyanobenzenesulfonyl)-L-prolyl-L-4-(N,N-dimethylcarbamyloxy)phenylalanine isopropyl ester
N-(4-aminobenzenesulfonyl)-L-prolyl-L-4-(N,N-dimethylcarbamyloxy)phenylalanine
N-(toluene-4-sulfonyl)-L-4-oxoprolyl-L-4-(4-methylpiperazin-1-ylcarbonyloxy)phenylalanine tert-butyl ester
N-(toluene-4-sulfonyl)-L-prolyl-L-4-[3-(hydroxymethyl)piperidin-1-ylcarbonyloxy]phenylalanine
N-(toluene-4-sulfonyl)-L-(4,4-difluoro)prolyl-L-4-(N,N-dimethylcarbamyloxy)phenylalanine isopropyl ester
N-(toluene-4-sulfonyl)-L-(4,4-difluoro)prolyl-L-4-(N,N-dimethylcarbamyloxy)phenylalanine
N-(toluene-4-sulfonyl)-L-prolyl-L-(4-benzoylpiperazin-1-ylcarbonyloxy)phenylalanine isopropyl ester
N-(1-methyl-1H-imidazole-4-sulfonyl)-L-prolyl-L-4-(N,N-dimethylcarbamyloxy)phenylalanine isopropyl ester
N-(toluene-4-sulfonyl)-L-4-(thiomorpholin-4-ylcarbonyloxy)prolyl-L-4-(thiomorpholin-4-ylcarbonyloxy)phenylalanine
N-(4-cyanobenzenesulfonyl)-L-prolyl-L-4-(thiomorpholin-4-ylcarbonyloxy)phenylalanine isopropyl ester
N-(4-amidinobenzenesulfonyl)-L-prolyl-L-4-(N,N-dimethylcarbamyloxy)phenylalanine methyl ester
N-(toluene-4-sulfonyl)-L-4-oxoprolyl-L-4-(thiomorpholin-4-ylcarbonyloxy)phenylalanine tert-butyl ester
N-(toluene-4-sulfonyl)-L-4-hydroxyprolyl-L-4-(thiomorpholin-4-ylcarbonyloxy)phenylalanine
N-(toluene-4-sulfonyl)-L-prolyl-L-(4-benzoylpiperazin-1-ylcarbonyloxy)phenylalanine
N-(4-amidinobenzenesulfonyl)-L-prolyl-L-4-(thiomorpholin-4-ylcarbonyloxy)phenylalanine methyl ester
N-(3-fluorobenzenesulfonyl)-L-prolyl-L-4-(N,N-dimethylcarbonyloxy)phenylalanine
N-(toluene-4-sulfonyl)-L-prolyl-L-4-[N-methyl-N-(2-(N′-methyl-N′-toluenesulfonyl-amino)ethyl)carbamyloxy]phenylalanine isopropyl ester
N-(toluene-4-sulfonyl)-L-prolyl-L-4-[N-(2-(N,N-phenylaminocarbonyloxy)ethyl)carbamyloxy)]phenylalanine isopropyl ester
N-(4-fluorobenzenesulfonyl)-L-4-(trans-hydroxy)prolyl-L-4-(N,N-dimethylcarbamyloxy)phenylalanine isopropyl ester
N-(4-fluorobenzenesulfonyl)-L-4-(trans-hydroxy)prolyl-L-4-(N,N-dimethylcarbamyloxy)phenylalanine tert-butyl ester
N-(4-amidinobenzenesulfonyl)-L-prolyl-L-4-(N,N-dimethylcarbamyloxy)phenylalanine
N-(toluene-4-sulfonyl)-L-(pyrazin-3-carbonyl)-L-4-(N,N-dimethylcarbamyloxy)phenylalanine tert-butyl ester
N-(4-fluorobenzenesulfonyl)-L-(5,5-dimethyl)thiaprolyl-L-4-(2-hydroxymethylpyrrolidin-1-ylcarbonyloxy)phenylalanine tert-butyl ester
N-(4-fluorobenzenesulfonyl)-L-(5,5-dimethyl)thiaprolyl-L-4-(2-hydroxymethylpyrrolidin-1-ylcarbonyloxy)phenylalanine
N-(toluene-4-sulfonyl)-L-prolyl-L-4-(2-methoxycarbonylpyrrolidin-1-ylcarbonyloxy)phenylalanine tert-butyl ester
N-(4-fluorobenzenesulfonyl)-L-(5,5-dimethyl)thiaprolyl-L-3-chloro-4-(thiomorpholin-4-ylcarbonyloxy)phenylalanine
N-(4-fluorobenzenesulfonyl)-L-(5,5-dimethyl)thiaprolyl-L-4-(4-(2-pyridyl)piperazin-1-ylcarbonyloxy)]phenylalanine
N-(4-fluorobenzenesulfonyl)-L-(5,5-dimethyl)thiaprolyl-L-4-(4-(2-pyridyl)piperazin-1-ylcarbonyloxy)]phenylalanine tert-butyl ester
N-(toluene-4-sulfonyl)-L-(4-hydroxy)prolyl-L-4-(thiomorpholin-4-ylcarbonyloxy)phenylalanine tert-butyl ester
N-(toluene-4-sulfonyl)-L-prolyl-L-4-(N,N-dimethylcarbamyloxy)phenylalanine 2-(2-methoxyethoxy)ethyl ester
N-(4-fluorobenzenesulfonyl)-L-(5,5-dimethyl)thiaprolyl-L-4-(4-(2-pyrimidyl)piperazin-1-ylcarbonyloxy)]phenylalanine tert-butyl ester
N-(4-fluorobenzenesulfonyl)-L-(5,5-dimethyl)thiaprolyl-L-3-fluoro-4-(N,N-dimethylcarbanyloxy)phenylalanine isopropyl ester
N-(toluene-4-sulfonyl)-L-(1-methanesulfonylpyrazin-3-carbonyl)-L-4-(N,N-dimethylcarbamyloxy)phenylalanine tert-butyl ester
N-(4-bromobenzenesulfonyl)-L-(5,5-dimethyl)thiaprolyl-L-4-(N,N-dimethylcarbamyloxy)phenylalanine tert-butyl ester
N-(4-bromobenzenesulfonyl)-L-(5,5-dimethyl)thiaprolyl-L-4-(N,N-dimethylcarbamyloxy)phenylalanine
N-(toluene-4-sulfonyl)-L-(4-hydroxy)prolyl-L-4-(4-methylpiperazin-1-ylcarbonyloxy)phenylalanine
N-(4-fluorobenzenesulfonyl)-L-(5,5-dimethyl)thiaprolyl-L-4-(4-(2-pyrimidyl)piperazin-1-ylcarbonyloxy)]phenylalanine
N-(4-fluorobenzenesulfonyl)-L-(5,5-dimethyl)thiaprolyl-L-4-(4-(2-pyridyl)piperazin-1-ylcarbonyloxy)]phenylalanine isopropyl ester
N-(4-fluorobenzenesulfonyl)thiazolidinyl-2-carbonyl-L-4-(N,N-dimethylcarbamyloxy)phenylalanine
N-(4-fluorobenzenesulfonyl)thiazolidinyl-2-carbonyl-L-4-(N,N-dimethylcarbamyloxy)phenylalanine tert-butyl ester
N-(toluene-4-sulfonyl)-L-(4-oxo)prolyl-L-4-(thiomorpholin-4-ylcarbonyloxy)phenylalanine
N-(toluene-4-sulfonyl)-L-(4-oxo)prolyl-L-4-(4-methylpiperazin-1-ylcarbonyloxy)phenylalanine
N-(4-fluorobenzenesulfonyl)thiazolidinyl-2-carbonyl-L-4-(4-(2-pyridyl)piperazin-1-ylcarbonyloxy)]phenylalanine
N-(4-nitrobenzenesulfonyl)-L-prolyl-L-4-(4-(2-pyridyl)piperazin-1-ylcarbonyloxy)]phenylalanine tert-butyl ester
N-(4-fluorobenzenesulfonyl)thiazolidinyl-2-carbonyl-L-4-(4-(2-pyridyl)piperazin-1-ylcarbonyloxy)]phenylalanine tert-butyl ester
N-(4-bromobenzenesulfonyl)-L-(5,5-dimethyl)thiaprolyl-L-4-(4-(2-pyridyl)piperazin-1-ylcarbonyloxy)]phenylalanine
N-(toluene-4-sulfonyl)-L-prolyl-L-4-(4-(N-phenylthiocarbonyl)piperazin-1-ylcarbonyloxy)]phenylalanine isopropyl ester
N-(4-fluorobenzenesulfonyl)thiazolidinyl-2-carbonyl-L-4-(4-methylhomopiperazin-1-ylcarbonyloxy)phenylalanine tert-butyl ester
N-(toluene-4-sulfonyl)-L-4-(methanesulfonyloxy)prolyl-L-4-(N,N-dimethylcarbamyloxy)phenylalanine tert-butyl ester
N-(4-aminocarbonylbenzenesulfonyl)-L-prolyl-L-4-(N,N-dimethylcarbamyloxy)phenylalanine
N-(4-aminocarbonylbenzenesulfonyl)-L-prolyl-L-4-(thiomorpholin-4-ylcarbonyloxy)phenylalanine
N-(4-amidinobenzenesulfonyl)-L-prolyl-L-4-(thiomorpholin-4-ylcarbonyloxy)phenylalanine
N-(4-nitrobenzenesulfonyl)-L-prolyl-L-4-(4-(2-pyridyl)piperazin-1-ylcarbonyloxy)]phenylalanine
N-(4-fluorobenzenesulfonyl)-L-(5,5-dimethyl)thiaprolyl-L-3-chloro-4-(4-(2-pyridyl)piperazin-1-ylcarbonyloxy)]phenylalanine ethyl ester
N-(4-fluorobenzenesulfonyl)-L-(5,5-dimethyl)thiaprolyl-L-3-chloro-4-(4-(2-pyridyl)piperazin-1-ylcarbonyloxy)phenylalanine
N-(4-fluorobenzenesulfonyl)thiazolidinyl-2-carbonyl-L-4-(4-methylhomopiperazin-1-ylcarbonyloxy)phenylalanine
N-(1-methylpyrazole-3-sulfonyl)-L-(5,5-dimethyl)thiaprolyl-L-3-chloro-4-(N,N-dimethylcarbamyloxy)phenylalanine isopropyl ester
N-(1-methylimidazole-4-sulfonyl)-L-prolyl-L-4-(4-(2-pyridyl)piperazin-1-ylcarbonyloxy)phenylalanine isopropyl ester
N-(1-methylimidazole-4-sulfonyl)-L-prolyl-L-4-(4-(2-pyridyl)piperazin-1-ylcarbonyloxy)phenylalanine tert-butyl ester
N-(toluene-4-sulfonyl)-L-prolyl-L-4-(4-(2-pyridyl)piperazin-1-ylcarbonyloxy)phenylalanine
N-(toluene-4-sulfonyl)-L-prolyl-L-4-(4-(2-pyridyl)piperazin-1-ylcarbonyloxy)phenylalanine tert-butyl ester
N-(toluene-4-sulfonyl)-L-prolyl-L-4-(4-(2-pyridyl)piperazin-1-ylcarbonyloxy)phenylalanine isopropyl ester
N-(4-fluorobenzenesulfonyl)-L-prolyl-L-4-(4-(2-pyridyl)piperazin-1-ylcarbonyloxy)phenylalanine isopropyl ester
N-(4-fluorobenzenesulfonyl)-L-prolyl-L-4-(4-(2-pyridyl)piperazin-1-ylcarbonyloxy)phenylalanine tert-butyl ester
N-(toluene-4-sulfonyl)-L-(1-methanesulfonylpyrazin-3-carbonyl)-L-4-(N,N-dimethylcarbamyloxy)phenylalanine
N-(toluene-4-sulfonyl)-L-4-(methanesulfonyloxy)prolyl-L-4-(N,N-dimethylcarbamyloxy)phenylalanine
N-(4-bromobenzenesulfonyl)-L-(5,5-dimethyl)thiaprolyl-L-4-(4-(2-pyridyl)piperazin-1-ylcarbonyloxy)phenylalanine tert-butyl ester
N-(4-trifluoromethoxybenzenesulfonyl)-L-(5,5-dimethyl)thiaprolyl-L-4-(N,N-dimethylcarbamyloxy)phenylalanine
N-(4-trifluoromethoxybenzenesulfonyl)-L-(5,5-dimethyl)thiaprolyl-L-4-(N,N-dimethylcarbamyloxy)phenylalanine tert-butyl ester
N-(4-trifluoromethoxybenzenesulfonyl)-L-(5,5-dimethyl)thiaprolyl-L-4-(4-(2-pyridyl)piperazin-1-ylcarbonyloxy)phenylalanine tert-butyl ester
N-(4-fluorobenzenesulfonyl)-L-prolyl-L-4-(4-(2-pyridyl)piperazin-1-ylcarbonyloxy)phenylalanine
N-(4-fluorobenzenesulfonyl)-L-(4-hydroxy)prolyl-L-4-(4-(2-pyridyl)piperazin-1-ylcarbonyloxy)phenylalanine
N-(4-trifluoromethoxybenzenesulfonyl)-L-(5,5-dimethyl)thiaprolyl-L-4-(4-(2-pyridyl)piperazin-1-ylcarbonyloxy)phenylalanine
N-(1-methylimidazole-4-sulfonyl)-L-prolyl-L-3-chloro-4-(N,N-dimethylcarbamyloxy)phenylalanine
N-(1-methylimidazole-4-sulfonyl)-L-prolyl-L-3-chloro-4-(N,N-dimethylcarbamyloxy)phenylalanine isopropyl ester
N-(1-methylimidazole-4-sulfonyl)-L-prolyl-L-4-(4-(2-pyridyl)piperazin-1-ylcarbonyloxy)phenylalanine
N-(1-methylimidazole-4-sulfonyl)-L-(5,5-dimethyl)thiaprolyl-L-4-(4-(2-pyridyl)piperazin-1-ylcarbonyloxy)phenylalanine
N-(1-methylpyrazole-3-sulfonyl)-L-prolyl-L-4-(4-(2-pyridyl)piperazin-1-ylcarbonyloxy)phenylalanine
N-(1-methylpyrazole-3-sulfonyl)-L-prolyl-L-4-(4-(2-pyridyl)piperazin-1-ylcarbonyloxy)phenylalanine isopropyl ester
N-(1-methylpyrazole-3-sulfonyl)-L-prolyl-L-4-(4-(2-pyridyl)piperazin-1-ylcarbonyloxy)phenylalanine tert-butyl ester
N-(1-methylpyrazole-3-sulfonyl)-L-(5,5-dimethyl)thiaprolyl-L-4-(4-(2-pyridyl)piperazin-1-ylcarbonyloxy)phenylalanine tert-butyl ester
N-(1-methylimidazole-4-sulfonyl)-L-prolyl-L-3-chloro-4-(4-(2-pyridyl)piperazin-1-ylcarbonyloxy)phenylalanine isopropyl ester
N-(1-methylpyrazole-3-sulfonyl)-L-(5,5-dimethyl)thiaprolyl-L-4-(N,N-dimethylcarbamyloxy)phenylalanine 2-phenoxyethyl ester
N-(1-methylpyrazole-3-sulfonyl)-L-(5,5-dimethyl)thiaprolyl-L-3-chloro-4-(4-(2-pyridyl)piperazin-1-ylcarbonyloxy)phenylalanine
N-(1-methylpyrazole-3-sulfonyl)-L-(5,5-dimethyl)thiaprolyl-L-3-chloro-4-(4-(2-pyridyl)piperazin-1-ylcarbonyloxy)phenylalanine ethyl ester
N-(3-chloro-1,5-dimethylpyrazole-3-sulfonyl)-L-(5,5-dimethyl)thiaprolyl-L-3-chloro-4-(4-(5-trifluoromethyl-2-pyridyl)piperazin-1-ylcarbonyloxy)phenylalanine
   and pharmaceutically acceptable salts thereof.

Preferred compounds of formula I above include those set forth in Table 1 below:

TABLE 1

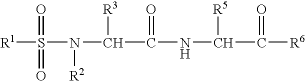

| R¹ | R² | R³ | R⁵ | R⁶ |
|---|---|---|---|---|
| p-CH₃-φ- | R²/R³ = cyclic | | p-[(4-methylpiperazin-1-yl)C(O)O-]benzyl- | —OCH₂CH₃ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OCH₂CH₃ |
| | 3 carbon atoms | | | |
| | (L-pyrrolidinyl) | | | |
| p-CH₃-φ- | R²/R³ = cyclic | | p-[(4-methylpiperazin-1-yl)C(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(4-methylpiperazin-1-yl)C(O)O-]benzyl- | —O-n-butyl |
| | 3 carbon atoms | | | |
| | (L-pyrrolidinyl) | | | |
| p-CH₃-φ- | R²/R³ = cyclic | | p-[(4-methylpiperazin-1-yl)C(O)O-]benzyl- | —O-cyclopentyl |
| | 3 carbon atoms | | | |
| | (L-pyrrolidinyl) | | | |
| p-CH₃-φ- | R²/R³ = cyclic | | p-[(4-methylpiperazin-1-yl)C(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(4-methylpiperazin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —O-n-butyl |
| | 3 carbon atoms | | | |
| | (L-pyrrolidinyl) | | | |
| p-CH₃-φ- | R²/R³ = cyclic | | p-[(CH₃)₂NC(O)O-]benzyl- | —O-cyclopentyl |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OH |
| | 3 carbon atoms | | | |
| | (L-pyrrolidinyl) | | | |
| p-CH₃-φ- | R²/R³ = cyclic | | p-[(piperidin-4-yl)C(O)O-]benzyl- | —OCH₂CH₃ |
| | 3 carbon atoms | | | |
| | (L-pyrrolidinyl) | | | |
| p-CH₃-φ- | R²/R³ = cyclic | | p-[(1-methylpiperidin-4-yl)C(O)O-]benzyl- | —OCH₂CH₃ |
| | 3 carbon atoms | | | |
| | (L-pyrrolidinyl) | | | |
| φ-CH₂— | R²/R³ = cyclic | | p-[(4-methylpiperazin-1-yl)C(O)O-]benzyl- | —OH |
| | 3 carbon atoms | | | |
| | (L-pyrrolidinyl) | | | |
| p-CH₃-φ- | R²/R³ = cyclic | | m-[(CH₃)₂NC(O)O-]benzyl- | —OCH₂CH₃ |
| | 3 carbon atoms | | | |
| | (L-pyrrolidinyl) | | | |
| p-CH₃-φ- | R²/R³ = cyclic | | p-[(1-Boc-4-phenylpiperidin-4-yl)- | —OCH₂CH₃ |
| | 3 carbon atoms | | C(O)O-]benzyl- | |
| | (L-pyrrolidinyl) | | | |
| p-CH₃-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5 -dimethylthiazolidin-4-yl) | | | |
| p-CH₃-φ- | R²/R³ = cyclic | | p-[(CH₃)₂NC(O)O-]benzyl- | —OC(CH₃)₃ |
| | —CH₂—S—C(CH₃)₂— | | | |
| | (L-5,5-dimethlthiazolidin-4-yl) | | | |
| p-CH₃-φ- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| p-CH₃-φ- | R²/R³ = cyclic | | p-[(CH₃)₂NC(O)O-]benzyl- | —OC(CH₃)₃ |
| | —CH₂CH₂—SO₂—CH₂— | | | |
| | (L-1,1-dioxothiomorpholin-3-yl) | | | |
| p-CH₃-φ- | R²/R³ = cyclic | | p-[(CH₃)₂NC(O)O-]benzyl- | —OH |
| | —CH₂CH₂—SO₂—CH₂— | | | |
| | (L-1,1-dioxothiomorpholin-3-yl) | | | |
| p-CH₃-φ- | R²/R³ = cyclic | | p-[(4-methylpiperazin-1-yl)C(O)O-]benzyl- | —OC(CH₃)₃ |
| | —CH₂—S—C(CH₃)₂— | | | |
| | (L-5,5-dimethylthiazolidin-4-yl) | | | |
| p-CH₃-φ- | R²/R³ = cyclic | | p-[(4-methylpiperazin-1-yl)C(O)O-]benzyl- | —OH |
| | —CH₂—S—C(CH₃)₂— | | | |
| | (L-5,5-dimethylthiazolidin-4-yl) | | | |
| p-CH₃-φ- | CH₃— | H | p-[(CH₃)₂NC(O)O-]benzyl- | —OCH(CH₃)₂ |
| p-CH₃-φ- | CH₃— | H | p-[(CH₃)₂NC(O)O-]benzyl- | —OC(CH₃)₃ |
| p-CH₃-φ- | CH₃— | H | p-[(CH₃)₂NC(O)O-]benzyl- | —OH |
| 1-methyl- | R²/R³ = cyclic | | p-[(CH₃)₂NC(O)O-]benzyl- | —OC(CH₃)₃ |
| imidazol-4-yl | 3 carbon atoms | | | |
| | (L-pyrrolidinyl) | | | |
| p-NH₂-φ- | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ- | CH₃— | H | p-[(morpholin-4-yl)C(O)O-]benzyl- | —OC(CH₃)₃ |
| p-CH₃-φ- | R²/R³ = cyclic | | p-[(morpholin-4-yl)C(O)O-]benzyl- | —OC(CH₃)₃ |
| | 3 carbon atoms | | | |
| | (L-pyrrolidinyl) | | | |
| φ-CH₂— | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| p-CH₃-φ- | R²/R³ = cyclic | | p-[(CH₃)₂NC(O)O-]benzyl- | —OH |
| | —CH₂CH₂—NH—CH₂— | | | |
| | (L-piperazinyl) | | | |
| φ-CH₂— | R²/R³ = cyclic | | p-[(CH₃)₂NC(O)O-]benzyl- | —OC(CH₃)₃ |
| | —CH₂—S—C(CH₃)₂— | | | |
| | (L-5,5-dimethylthiazolidin-4-yl) | | | |
| p-CH₃-φ- | R²/R³ = cyclic | | p-[(CH₃)₂NC(O)O-]benzyl- | —OC(CH₃)₃ |
| | —CH₂CH₂—NH—CH₂— | | | |
| | (L-piperazinyl) | | | |
| p-CH₃-φ- | R²/R³ = cyclic | | p-[(CH₃)₂NC(O)O-]benzyl- | —OC(CH₃)₃ |
| | —CH₂CH₂-(Cbz)NHCH₂— | | | |
| | [L-4-N-(Cbz)-piperazinyl] | | | |
| p-CH₃-φ- | CH₃— | H | p-[(piperidin-1-yl)C(O)O-]benzyl- | —OH |
| p-CH₃-φ- | R²/R³ = cyclic | | p-[(morpholin-4-yl)C(O)O-]benzyl- | —OC(CH₃)₃ |
| | —CH₂CH₂—SO₂—CH₂— | | | |
| | (L-1,1-dioxothiomorpholin-3-yl) | | | |
| p-CH₃-φ- | R²/R³ = cyclic | | p-[(morpholin-4-yl)C(O)O-]benzyl- | —OH |
| | —CH₂CH₂—SO₂—CH₂— | | | |
| | (L-1,1-dioxothiomorpholin-3-yl) | | | |
| 1-methyl- | R²/R³ = cyclic | | p-[(CH₃)₂NC(O)O-]benzyl- | —OC(CH₃)₃ |
| pyrazol-4-yl | 3 carbon atoms | | | |
| | (L-pyrrolidinyl) | | | |
| p-F-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ | —CH₃ | H | p-[(4-methylpiperazin-1-yl)C(O)O-]benzyl- | —OC(CH₃)₃ |
| p-CH₃-φ | R²/R³ = cyclic | | p-[(CH₃)₂NC(O)O-]benzyl- | —OC(CH₃)₃ |
| | —CH₂—SO₂—C(CH₃)₂— | | | |
| | (L-1,1-dioxo-5,5- | | | |
| | dimethylthiazolidin-4-yl) | | | |
| 1-methyl- | R²/R³ = cylic | | p-[(CH₃)₂NC(O)O-]benzyl- | —OH |
| pyrazol-4-yl | 3 carbon atoms | | | |
| | (L-pyrrolidinyl) | | | |
| p-CH₃-φ | R²/R³ = cyclic | | p-[(CH₃)₂NC(O)O-]benzyl- | —OH |
| | —CH₂—SO₂—C(CH₃)₂— | | | |
| | (L-1,1-dioxo-5,5- | | | |
| | dimethylthiazolidin-4-yl) | | | |
| p-F-φ | R²/R³ = cyclic | | p-[(CH₃)₂NC(O)O-]benzyl- | —OH |
| | 3 carbon atoms | | | |
| (L-pyrrolidinyl) | | | | |
| 3-pyridyl | R²/R³ = cyclic | | p-[(CH₃)₂NC(O)O-]benzyl- | —OH |
| | 3 carbon atoms | | | |
| | (L-pyrrolidinyl) | | | |
| p-CH₃-φ | R²/R³ = cyclic | | p-[(4-methylpiperazin-1-yl)C(O)O-]benzyl- | —OC(CH₃)₃ |
| | 3 carbon atoms | | | |
| | (D-pyrrolidinyl) | | | |
| p-CH₃-φ | —CH₃ | —CH₃ | p-[(4-methylpiperazin-1-yl)C(O)O-]benzyl- | —OC(CH₃)₃ |
| p-nitro-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ | —CH₃ | H | p-[(thiomorpholin-4-yl)C(O)O-]benzyl- | —OC(CH₃)₃ |
| p-CH₃-φ | —CH₃ | —CH₃ | p-[(4-methylpiperazin-1-yl)C(O)O-]benzyl- | —OH |
| p-CH₃-φ | R²/R³ = cyclic | | p-[(thiomorpholin-4-yl sulfone)-C(O)O-]benzyl- | —OH |
| | 3 carbon atoms | | | |
| | (L-pyrrolidinyl) | | | |
| p-CH₃-φ | R²/R³ = cyclic | | p-[(thiomorpholin-4-yl)C(O)O-]benzyl- | —OH |
| | 3 carbon atoms | | | |
| | (L-pyrrolidinyl) | | | |
| p-CH₃-φ | R²/R³ = cyclic | | p-[(piperidin-1-yl)C(O)O-]benzyl- | —OH |
| | 3 carbon atoms | | | |
| | (L-pyrrolidinyl) | | | |
| p-CH₃-φ | R²/R³ = cyclic | | p-[(pyrrolidin-1-yl)C(O)O-]benzyl- | —OH |
| | 3 carbon atoms | | | |
| | (L-pyrrolidinyl) | | | |
| p-CH₃-φ | R²/R³ = cyclic | | p-[(morpholin-4-yl)C(O)O-]benzyl- | —OH |
| | 3 carbon atoms | | | |
| | (L-pyrrolidinyl) | | | |
| p-CH₃-φ | R²/R³ = cyclic | | p-[(4-methylpiperazin-1-yl)C(O)O-]benzyl- | —OCH₂C(CH₃)₃ |
| | 3 carbon atoms | | | |
| | (L-pyrrolidinyl) | | | |
| p-CH₃-φ | R²/R³ = cyclic | | p-[(CH₃)₂NC(O)O-]benzyl- | —OCH₂C(CH₃)₃ |
| | 3 carbon atoms | | | |
| | (L-pyrrolidinyl) | | | |
| p-CH₃-φ | R²/R³ = cyclic | | p-[(1-Boc-piperazin-4-yl)C(O)O-]benzyl- | —OCH₂CH₃ |
| | 3 carbon atoms | | | |
| | (L-pyrrolidinyl) | | | |
| p-CH₃-φ | R²/R³ = cyclic | | p-[(morpholin-4-yl)C(O)O-]benzyl- | —OCH₂CH₃ |
| | 3 carbon atoms | | | |
| | (L-pyrrolidinyl) | | | |
| p-CH₃-φ | —CH₃ | H | p-[(thiomorpholin-4-yl sulfone)-C(O)O-]benzyl- | —OC(CH₃)₃ |
| p-CH₃-φ | —CH₃ | H | p-[(thiomorpholin-4-yl)C(O)O-]benzyl- | —OH |
| p-CH₃-φ | —CH₃ | —CH₃ | p-[(CH₃)₂NC(O)O-]benzyl- | —OC(CH₃)₃ |
| p-CH₃-φ | R²/R³ = cyclic | | p-[(CH₃)₂NC(O)O-]benzyl- | —OC(CH₃)₃ |
| | —CH₂—CH₂—S—CH₂— | | | |
| | (L-thiomorpholin-3-yl) | | | |
| p-CH₃-φ | —CH₃ | H | p-[(thiomorpholin-4-yl sulfone)-C(O)O-]benzyl- | —OH |
| p-CH₃-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| p-CH₃-φ | R²/R³ = cyclic | | p-[(morpholin-4-yl)C(O)O-]benzyl- | —OC(CH₃)₃ |
| | —CH₂—CH₂—SO₂—CH₂— | | | |
| | (L-1,1-dioxothiomorpholin-3-yl) | | | |
| p-CH₃-φ | —CH₃ | —CH₃ | p-[(CH₃)₂NC(O)O-]benzyl- | —OH |
| p-F-φ | R²/R³ = cyclic | | p-[(CH₃)₂NC(O)O-]benzyl- | —OC(CH₃)₃ |
| | —CH₂—CH₂—S—CH₂— | | | |
| | (L-thiomorpholin-3-yl) | | | |
| p-F-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| pyridin- | R²/R³ = cyclic | | p-[(CH₃)₂NC(O)O-]benzyl- | —OC(CH₃)₃ |
| 3-yl | 3 carbon atoms | | | |
| | (L-pyrrolidinyl) | | | |
| p-nitro-φ | R²/R³ = cyclic | | p-[(CH₃)₂NC(O)O-]benzyl- | —OH |
| | 3 carbon atoms | | | |
| | (L-pyrrolidinyl) | | | |
| p-N≡C-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ | R²/R³ = cyclic | | p-[(CH₃)₂NC(O)O-]benzyl- | —OH |
| | —CH₂—CH₂—SO₂—CH₂— | | | |
| | (L-1,1-dioxothiomorpholin-3-yl) | | | |
| p-CH₃-φ | R²/R³ = cyclic | | p-[(CH₃)₂NC(O)O-]benzyl- | —OC(CH₃)₃ |
| | —CH₂—SO₂—CH₂— | | | |
| | (L-1,1-dioxothiazolidin-4-yl) | | | |
| p-F₃C-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| 1-methyl- | R²/R³ = cyclic | | p-[(CH₃)₂NC(O)O-]benzyl- | —OH |
| pyrazol-4- | 3 carbon atoms | | | |
| yl | (L-pyrrolidinyl) | | | |
| p-CH₃-φ | R²/R³ = cyclic | | p-[(CH₃)₂NC(O)O-]benzyl- | —OH |
| | —CH₂—SO₂—CH₂— | | | |
| | (L-1,1-dioxothiazolidin-4-yl) | | | |
| p-F-φ | R²/R³ = cyclic | | p-[(CH₃)₂NC(O)O-]benzyl- | —OH |
| | —CH₂—S—CH₂— | | | |
| | (L-thiazolidin-4-yl) | | | |
| p-CH₃-φ | R²/R³ = cyclic | | p-[(CH₃)₂NC(O)O-]benzyl- | 2,4-dioxo- |
| | 3 carbon atoms | | | tetrahydrofuran |
| | (L-pyrrolidinyl) | | | 3-yl (3,4-enol) |
| p-CH₃-φ | R²/R³ = cyclic | | p-[(piperazin-4-yl)C(O)O-]benzyl- | —OH |
| | 3 carbon atoms | | | |
| | (L-pyrrolidinyl) | | | |
| p-CH₃-φ | R²/R³ = cyclic | | p-[(1-Boc-piperazin-4-yl)C(O)O-]benzyl- | —OC(CH₃)₃ |
| | 3 carbon atoms | | | |
| | (L-pyrrolidinyl) | | | |
| p-CH₃-φ | R²/R³ = cyclic | | p-[(piperazin-4-yl)C(O)O-]benzyl- | —OCH₂CH₃ |
| | 3 carbon atoms | | | |
| | (L-pyrrolidinyl) | | | |
| p-CH₃-φ | R²/R³ = cyclic | | p-[(4-acetylpiperazin-1-yl)C(O)O-]benzyl- | —OCH₂CH₃ |
| | 3 carbon atoms | | | |
| | (L-pyrrolidinyl) | | | |
| p-CH₃-φ | R²/R³ = cyclic | | p-[(4-methanesulfonylpiperazin-1-yl)- | —OCH₂CH₃ |
| | 3 carbon atoms | | C(O)O-]benzyl- | |
| | (L-pyrrolidinyl) | | | |
| p-CH₃-φ | R²/R³ = cyclic | | 3-nitro-4-[(morpholin-4-yl)-C(O)O-]benzyl- | —OH |
| | 3 carbon atoms | | | |
| | (L-pyrrolidinyl) | | | |
| p-CH₃-φ | R²/R³ = cyclic | | p-[(1-Boc-piperazin-4-yl)C(O)O-]benzyl- | —OH |
| | 3 carbon atoms | | | |
| | (L-pyrrolidinyl) | | | |
| p-CH₃-φ | —CH₃ | —C(CH₃)₃ | p-[(4-methylpiperazin-1-yl)C(O)O-]benzyl- | —OC(CH₃)₃ |
| p-F-φ | R²/R³ = cyclic | | p-[(CH₃)₂NC(O)O-]benzyl- | —OC(CH₃)₃ |
| | —CH₂—S—C(CH₃)₂— | | | |
| | (L-5,5-dimetlylthiazolidin-4-yl) | | | |
| p-F-φ | R²/R³ = cyclic | | p-[(1,1-dioxothiomorpholin-4-yl)-C(O)O-]benzyl- | —OC(CH₃)₃ |
| | —CH₂—CH₂—SO₂—CH₂— | | | |
| | (L-1,1-dioxothiomorpholin-3-yl) | | | |
| p-CH₃-φ | R²/R³ = cyclic | | p-[(1,1-dioxothiomorpholin-4-yl)-C(O)O-]benzyl- | —OC(CH₃)₃ |
| | —CH₂—CH₂—SO₂—CH₂— | | | |
| | (L-1,1-dioxothiomorpholin-3-yl) | | | |
| p-F-φ | R²/R³ = cyclic | | p-[(1,1-dioxothiomorpholin-4-yl)-C(O)O-]benzyl- | —OC(CH₃)₃ |
| | 3 carbon atoms | | | |
| | (L-pyrrolidinyl) | | | |
| p-F-φ | R²/R³ = cyclic | | p-[(thiomorpholin-4-yl)C(O)O-]benzyl- | —OC(CH₃)₃ |
| | 3 carbon atoms | | | |
| | (L-pyrrolidinyl) | | | |
| p-F-φ | R²/R³ = cyclic | | p-[(morpholin-4-yl)C(O)O-]benzyl- | —OC(CH₃)₃ |
| | 3 carbon atoms | | | |
| | (L-pyrrolidinyl) | | | |
| p-F-φ | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| p-F-φ | R²/R³ = cyclic | | p-[(morpholin-4-yl)C(O)O-]benzyl- | —OC(CH₃)₃ |
| | —CH₂—CH₂SO₂—CH₂— | | | |
| | (L-1,1-dioxothiomorpholin-3-yl) | | | |
| p-F₃CO-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ | R²/R³ = cyclic | | p-[(CH₃)₂NC(O)O-]benzyl- | —OCH(CH₃)₂ |
| | —CH₂—CH₂—SO₂—CH₂— | | | |
| | (L-1,1-dioxothiomorpholin-3-yl) | | | |
| p-F-φ | R²/R³ = cyclic | | p-[(CH₃)₂NC(O)O-]benzyl- | —OC(CH₃)₃ |
| | —CH₂—SO₂—C(CH₃)₂— | | | |
| | (L-1,1-dioxo-5,5- | | | |
| | dimethylthiazolidin-4-yl) | | | |
| p-CH₃-φ | R²/R³ = cyclic | | p-[(CH₃)₂NC(O)O-]benzyl- | —OCH(CH₃)₂ |
| | —CH₂—SO₂—C(CH₃)₂— | | | |
| | (L-1,1-dioxo-5,5- | | | |
| | dimethylthiazolidin-4-yl) | | | |
| p-CH₃-φ | R²/R³ = cyclic | | p-[(morpholin-4-yl)C(O)O-]benzyl- | —OH |
| | —CH₂—CH₂—SO₂—CH₂— | | | |
| | (L-1,1-dioxothiomorpholin-3-yl) | | | |
| p-F-100 | R²/R³ = cyclic | | p-[(CH₃)₂NC(O)O-]benzyl- | —OH |
| | —CH₂—SO₂—C(CH₃)₂— | | | |
| | (L-1,1-dioxo-5,5- | | | |
| | dimethylthiazolidin-4-yl) | | | |
| pyrimidin- | R²/R³ = cyclic | | p-[(CH₃)₂NC(O)O-]benzyl- | —OH |
| 2-yl | 3 carbon atoms | | | |
| | (L-pyrrolidinyl) | | | |
| p-CH₃-φ | R²//R³ = cyclic | | p-[(4-methylpiperazin-1-yl)C(O)O-]benzyl- | —OC(CH₃)₃ |
| | —CH₂—CH₂—SO₂—CH₂— | | | |
| | (L-1,1-dioxothiomorpholin-3-yl) | | | |
| p-F-φ | R²/R³ = cyclic | | p-[(CH₃)₂NC(O)O-]benzyl- | —OC(CH₃)₃ |
| | —CH₂—SO₂—CH₂— | | | |
| | (L-1,1-dioxothiazolidin-4-yl) | | | |
| 2,5-dichloro- | R²/R³ = cyclic | | p-[(CH₃)₂NC(O)O-]benzyl- | —OC(CH₃)₃ |
| thien-3-yl | 3 carbon atoms | | | |
| | (L-pyrrolidinyl) | | | |
| p-CH₃C(O)NH-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-C(CH₃)₃-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| pyridin-2-yl | R²/R³ = cyclic | | p-[(CH₃)₂NC(O)O-]benzyl- | —OH |
| | 3 carbon atoms | | | |
| | (L-pyrrolidinyl) | | | |
| o-F-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| m-F-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| 2,4-difluoro-φ | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| p-CH₃C(O)NH-φ | R²/R³ = cyclic | | p-[(CH₃)₂NC(O)O-]benzyl- | —OH |
| | 3 carbon atoms | | | |
| | (L-pyrrolidinyl) | | | |
| p-C(F)₃-φ | R²/R³ = cyclic | | p-[(CH₃)₂NC(O)O-]benzyl- | —OH |
| | 3 carbon atoms | | | |
| | (L-pyrrolidinyl) | | | |
| p-F-φ | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-N≡C-φ | R²/R³ = cyclic | | p-[(CH₃)₂NC(O)O-]benzyl- | —OH |
| | 3 carbon atoms | | | |
| | (L-pyrrolidinyl) | | | |
| morpholin-4-yl | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ | R²/R³ = cyclic | | p-[(CH₃)₂NC(O)O-]benzyl- | —OC(CH₃)₃ |
| | —CH₂—CH₂—C(CH₃)₂— | | | |
| | (L-4,4-dimethyl pyrrolidinyl) | | | |
| p-CH₃-φ | R²/R³ = cyclic | | p-[(CH₃)₂NC(O)O-]benzyl- | —OCH(CH₃)₂ |
| | —CH₂—CH₂—C(CH₃)₂— | | | |
| | (L-4,4-dimethyl pyrrolidinyl) | | | |
| 1-methyl- | R²/R³ = cyclic | | p-[(CH₃)₂NC(O)O-]benzyl- | —OCH(CH₃)₂ |
| pyrazol-4-yl | 3 carbon atoms | | | |
| | (L-pyrrolidinyl) | | | |
| p-CH₃-φ | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-CH₃-φ | R²/R³ = cyclic | | p-[(thiomorpholin-4-yl)C(O)O-]benzyl- | —OC(CH₃)₃ |
| | 3 carbon atoms | | | |
| | (L-pyrrolidinyl) | | | |
| 1-methyl- | R²/R³ = cyclic | | p-[(CH₃)₂NC(O)O-]benzyl- | —OH |
| imidazol-4-yl | 3 carbon atoms | | | |
| | (L-pyrrolidinyl) | | | |
| p-CH₃-φ- | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| 1-methyl- | R²/R³ = cyclic | | p-[(CH₃)₂NC(O)O-]benzyl- | —OC(CH₃)₃ |
| pyrazol-4-yl | —CH₂—S—C(CH₃)₂— | | | |
| | (L-5,5-dimethylthiazolidin-4-yl) | | | |
| p-CH₃C(O)NH-φ | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-(CH₃)₃C-φ- | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| 1-methyl- | R²/R³ = cyclic | | p-[(CH₃)₂NC(O)O-]benzyl- | —OH |
| pyrazol-4-yl | —CH₂—S—C(CH₃)₂— | | | |
| | (L-5,5-dimethylthiazolidin-4-yl) | | | |
| pyridin-3-yl- | R²/R³ = cyclic | | p-[(CH₃)₂NC(O)O-]benzyl- | —OH |
| | 3 carbon atoms | | | |
| | (L-pyrrolidinyl) | | | |
| p-N≡C-φ- | R²/R³ = cyclic | | p-[(CH₃)₂NC(O)O-]benzyl- | —OH |
| | 3 carbon atoms | | | |
| | (L-pyrrolidinyl) | | | |
| p-F-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-CH₃-φ- | R²/R³ = cyclic | | p-[(1,4-dioxa-8-azaspiro[4.5]decan-8-yl)- | —OCH₂CH₃ |
| | 3 carbon atoms | | C(O)O-]benzyl- | |
| p-CH₃-φ- | R²/R³ = cyclic | | p-[(1,4-dioxa-8-azaspiro [4.5]decan-8- | —OH |
| | 3 carbon atoms | | yl)-C(O)O-]benzyl | |
| | (L-pyrrolidinyl) | | | |
| p-CH₃-φ- | R²/R³ = cyclic | | p-[(4-acetylpiperazin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(4-methanesulfonylpiperazin-1- | —OH |
| | 3 carbon atoms | | yl-C(O)O-]benzyl | |
| p-CH₃-φ- | R²/R³ = cyclic | | p-[(4-φ-piperazin-1-yl)C(O)(O)-]benzyl- | —OH |
| | 3 carbon atoms | | | |
| | (L-pyrrolidinyl) | | | |
| p-CH₃-φ- | R²/R³ = cyclic | | p-[(piperazin-1-yl)C(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OC(CH₃)₃ |
| | —CH₂CH₂—NH—CH₂— | | | |
| | (L-piperazinyl) | | | |
| p-F₃CO-φ- | R²/R³ = cyclic | | p-[(CH₃)₂NC(O)O-]benzyl- | —OH |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OC(CH₃)₃ |
| | —CH₂—CH₂—C(CH₃)₂— | | | |
| | (4,4-dimethyl pyrrolidinyl) | | | |
| p-CH₃-φ | R²/R³ = cyclic | | p-[(CH₃)₂NC(O)O-]benzyl- | —OCH(CH₃)₂ |
| | —CH₂—CH₂—C(CH₃)₂— | | | |
| | (4,4-dimethyl pyrrolidinyl) | | | |
| p-CH₃C(O)NH-φ- | R²/R³ = cyclic | | p-[(CH₃)₂NC(O)O-]benzyl- | —OH |
| | 3 carbon atoms | | | |
| | (L-pyrrolidinyl) | | | |
| o-F-φ- | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| morpholin- | R²/R³ = cyclic | | p-[(CH₃)₂NC(O)O-]benzyl- | —OC(CH₃)₃ |
| 4-yl- | 3 carbon atoms | | | |
| | (L-pyrrolidinyl) | | | |
| m-F-φ- | R²/R³ = cyclic | | p-[(CH₃)₂NC(O)O-]benzyl- | —OC(CH₃)₃ |
| | —CH₂—CH₂—SO₂—CH₂— | | | |
| | (L-1,1-dioxothiomorpholin-3-yl) | | | |
| 2,4- | R²/R³ = cyclic | | p-[(CH₃)₂NC(O)O-]benzyl- | —OC(CH₃)₃ |
| difluoro- | —CH₂—CH₂—SO₂—CH₂— | | | |
| φ- | (L-1,1-dioxothiomorpholin-3-yl) | | | |
| morpholin- | R²/R³ = cyclic | | p-[(CH₃)₂NC(O)O-]benzyl- | —OH |
| 4-yl- | 3 carbon atoms | | | |
| | (L-pyrrolidinyl) | | | |
| 1-methyl- | R²/R³ = cyclic | | p-[(CH₃)₂NC(O)O-]benzyl- | —OC(CH₃)₃ |
| pyrazol-4-yl- | —CH₂—CH₂—SO₂—CH₂— | | | |
| | (L-1,1-dioxothiomorpholin-3-yl) | | | |
| o-F-φ- | R²/R³ = cyclic | | p-[(CH₃)₂NC(O)O-]benzyl- | —OH |
| | —CH₂—CH₂—SO₂—CH₂— | | | |
| | (L-1,1-dioxothiomorpholin-3-yl) | | | |
| 2,4-difluoro-φ- | R²/R³ = cyclic | | p-[(CH₃)₂NC(O)O-]benzyl- | —OH |
| | —CH₂—CH₂—SO₂—CH₂— | | | |
| | (L-1,1-dioxothiomorpholin-3-yl) | | | |
| p-CH₃-φ- | R²/R³ = cyclic | | p-[(CH₃)₂NC(O)O-]benzyl- | —OH |
| | —CH₂—CH₂—S—CH₂— | | | |
| | (L-thiomorpholin-3-yl) | | | |
| pyridin-3-yl- | R²/R³ = cyclic | | p-[(CH₃)₂NC(O)O-]benzyl- | —OCH(CH₃)₂ |
| | —CH₂—S—C(CH₃)₂— | | | |
| | (L-1,1-dioxothiomorpholin-3-yl) | | | |
| m-F-φ- | R²/R³ = cyclic | | p-[(CH₃)₂NC(O)O-]benzyl- | —OH |
| | —CH₂—CH₂—SO₂—CH₂— | | | |
| | (L-1,1-dioxothiomorpholin-3-yl) | | | |
| pyridin-2-yl- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| 1-methyl- | R²/R³ = cyclic | | p-[(CH₃)₂NC(O)O-]benzyl- | —OH |
| pyrazol-4-yl | —CH₂—CH₂—SO₂—CH₂— | | | |
| | (L-1,1-dioxothiomorpholin-3-yl) | | | |
| p-CH₃-φ- | R²/R³ = cyclic | | p-[(4-methanesulfonylpiperazin-1-yl)- | —OC(CH₃)₃ |
| | 3 carbon atoms | | C(O)O-]benzyl- | |
| | (L-pyrrolidinyl) | | | |
| p-CH₃-φ- | R²/R³ = cyclic | | p-[(4-φ-piperazin-1-yl)C(O)(O)-]benzyl- | —OC(CH₃)₃ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —O—CH₂C(CH₃)₂— |
| | 3 carbon atoms | | NHC(O)OC(CH₃)₃ | |
| | (L-pyrrolidinyl) | | | |
| p-CH₃-φ- | R²/R³ = cyclic | | p-[(CH₃)₂NC(O)O-]benzyl- | —O—CH₂CH₂— |
| | 3 carbon atoms | | | (morpholin-4 yl) |
| | (L-pyrrolidinyl) | | | |
| p-CH₃-φ- | R²/R³ = cyclic | | p-[(4-acetylpiperazin-1-yl)C(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(CH₃)₂NCH₂CH₂N(CH₃)C(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(CH₃)₂NCH₂CH₂N(CH₃)C(O)O-]benzyl- | —OC(CH₃)₃ |
| | —CH₂—S—C(CH₃)₂— | | | |
| | (L-5,5-dimethylthiazolidin-4-yl) | | | |
| p-CH₃-φ- | R²/R³ = cyclic | | p-[(CH₃)₂NCH₂CH2N(CH₃)C(O)O-]benzyl- | —OH |
| | —CH₂—S—C(CH₃)₂— | | | |
| | (L-5,5-dimehylthiazolidin-4-yl) | | | |
| p-CH₃-φ- | R²/R³ = cyclic | | p-[(CH₃)₂NCH₂CH2N(CH₃)C(O)O-]benzyl- | —OH |
| | 3 carbon atoms | | | |
| | (L-pyrrolidinyl) | | | |
| p-CH₃-φ- | R²/R³ = cyclic | | p-[(4-hydroxypiperidin-1-yl)C(O)O-]benzyl- | —OC(CH₃)₃ |
| | 3 carbon atoms | | | |
| | (L-pyrrolidinyl) | | | |
| p-(CH₃)₃C-φ | R²/R³ = cyclic | | p-[(CH₃)₂NC(O)O-]benzyl- | —OH |
| | —CH₂—CH₂—SO₂—CH₂— | | | |
| | (L-1,1-dioxothiomorpholin-3-yl) | | | |
| p-CH₃-φ- | R²/R³ = cyclic | | p-[(thiomorpholin-4-yl)C(O)O-]benzyl- | —OC(CH₃)₃ |
| | —CH₂—S—C(CH₃)₂— | | | |
| | (L-5,5-dimethylthiazolidin-4-yl) | | | |
| 2,5-dichloro- | R²/R³ = =cyclic | | p-[(CH₃)₂NC(O)O-]benzyl- | —OH |
| thien-3-yl- | 3 carbon atoms | | | |
| | (L-pyrrolidinyl) | | | |
| p-CH₃O-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OH |
| | —CH₂—CH₂—C(CH₃)₂— | | | |
| | (4,4-dimethyl pyrrolidinyl) | | | |
| p-F-φ- | R²/R³ = cyclic | | 3-chloro-4-[(4-methylpiperazin-1-yl)C(O)O-]- | —OC(CH₃)₃ |
| | —CH₂—S—C(CH₃)₂— | | | |
| | (L-5,5-dimethylthiazolidin-4-yl) | | | |
| p-F-φ- | R²/R³ = cyclic | | 3-chloro-4-[(CH₃)₂NC(O)O-]-benzyl- | —OH |
| | —CH₂—S—C(CH₃)₂— | | | |
| | (L-5,5-dimethylthiazolidin-4-yl) | | | |
| p-F-φ- | R²/R³ = cyclic | | 3-chloro-4-[(thiomorpholin-4-yl)C(O)O-]-benzyl- | —OC(CH₃)₃ |
| | —CH₂—S—C(CH₃)₂— | | | |
| | (L-5,5-dimethylthiazolidin-4-yl) | | | |
| p-F-φ- | R²/R³ = cyclic | | 3-chloro-4-[(thiomorpholin-4-yl)C(O)O-]-benzyl- | —OC(CH₃)₃ |
| | —CH₂—S—C(CH₃)₂— | | | |
| | (L-5,5-dimethylthiazolidin-4-yl) | | | |
| p-CH₃-φ- | R²/R³ = cyclic | | p-[(morpholin-4-yl)-CH₂CH₂NHC(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(1,4-dioxa-8-azaspiro[4.5]decan-8-yl)- | —OC(CH₃)₃ |
| | 3 carbon atoms | | C(O)O-]benzyl- | |
| | (L-pyrrolidinyl) | | | |
| p-F-φ- | R²/R³ = cyclic | | 3-chloro-4-[(CH₃)₂NC(O)O-]-benzyl- | —OC(CH₃)₃ |
| | —CH₂—S—C(CH₃)₂— | | | |
| | (L-5,5-dimethylthiazolidin-4-yl) | | | |
| p-CH₃-φ- | R²/R³ = cyclic | | p-[(thiomorpholin-4-yl)C(O)O-]benzyl- | —OH |
| | —CH₂—S—C(CH₃)₂— | | | |
| | (L-5,5-dimethylthiazolidin-4-yl) | | | |
| pyridin-2-yl | R²/R³ = cyclic | | p-[(CH₃)₂NC(O)O-]benzyl- | —OH |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OCH(CH₃)₂ |
| | —CH₂—CH₂—(O)—CH₂— | | | |
| | (L-1-oxothiomorpholin-4-yl) | | | |
| 4-Cl-3-(NH₂— | R²/R³ = cyclic | | p-[(CH₃)₂NC(O)O-]benzyl- | —OCH(CH₃)₂ |
| SO₂-)-φ- | 3 carbon atoms | | | |
| | (L-pyrrolidinyl) | | | |
| p-F-φ- | R²/R³ = cyclic | | 3-chloro-4-[(thiomorpholin-4-yl)C(O)O-]-benzyl- | —OCH(CH₃)₂ |
| | —CH₂—S—C(CH₃)₂— | | | |
| | (L-5,5-dimethylthiazolidin-4-yl) | | | |
| p-F-φ- | R²/R³ = cyclic | | 3-chloro-4-[(CH₃)₂NC(O)O-]-benzyl- | —OCH(CH₃)₂ |
| | —CH₂—S—C(CH₃)₂— | | | |
| | (L-5,5-dimethylthiazolidin-4-yl) | | | |
| p-CH₃-φ- | R²/R³ = cyclic | | p-[HOCH₂CH₂N(CH₃)C(O)O-]benzyl- | —OC(CH₃)₃ |
| | 3 carbon atoms | | | |
| | (L-pyrrolidinyl) | | | |
| p-F-φ- | R²/R³ = cyclic | | p-[(2-(hydroxymethyl)pyrrolidin-1-yl)-C(O)O-]- | —OC(CH₃)₃ |
| | —CH₂—S—C(CH₃)₂— | | benzyl- | |
| | (L-5,5-dimethylthiazolidin-4-yl) | | | |
| p-F-φ- | R²/R³ = cyclic | | p-[(2-(hydroxymethyl)pyrrolidin-1-yl)-C(O)O-]- | —OH |
| | —CH₂—S—C(CH₃)₂— | benzyl- | | |
| | (L-5,5-dimethylthiazolidin-4-yl) | | | |
| p-CH₃-φ- | R²/R³ = cyclic | | p-[(2-(CH3OC(O)-)pyrrolidin-1-yl)-C(O)O-]- | —OC(CH₃)₃ |
| | 3 carbon atoms | | benzyl- | |
| | (L-pyrrolidinyl) | | | |
| p-CH₃-φ- | R²/R³ = cyclic | | p-[(4-(HC(O)O-)piperidin-1-yl)-C(O)O-]-benzyl- | —OH |
| | 3 carbon atoms | | | |
| | (L-pyrrolidinyl) | | | |
| p-CH₃-φ- | R²/R³ = cyclic | | p-[(4-(hydroxypiperidin-1-yl)-C(O)O-]- benzyl- | —OCH(CH₃)₂ |
| | 3 carbon atoms | | | |
| | (L-pyrrolidinyl) | | | |
| p-F-φ- | R²/R³ = cyclic | | 3-chloro-4-[(thiomorpholin-4-yl)C(O)O-]-benzyl- | —OH |
| | —CH₂—S—C(CH₃)₂— | | | |
| | (L-5,5-dimethylthiazolidin-4-yl) | | | |
| p-CH₃-φ- | R²/R³ = cyclic | | p-[(4-(CH₃CH₂OC(O)-)piperidin-1- | —OC(CH₃)₃ |
| | 3 carbon atoms | | C(O)O-]-benzyl | |
| | (L-pyrrolidinyl) | | | |
| p-CH₃-φ- | R²/R³ = cyclic | | p-[(4-(HOCH₂CH₂-)piperazin-1-yl)- | —OC(CH₃)₃ |
| | 3 carbon atoms | | C(O)O-]benzyl- | |
| | (L-pyrrolidinyl) | | | |
| p-F-φ- | R²/R³ = cyclic | | p-[(4-(pyridin-2-yl)piperazin-1-yl)- | —OH |
| | —CH₂—S—C(CH₃)₂— | | C(O)O-]benzyl- | |
| | (L-5,5-dimethylthiazolidin-4-yl) | | | |
| p-F-φ- | R²/R³ = cyclic | | p-[(4-(pyridin-2-yl)piperazin-1-yl)- | —OC(CH₃)₃ |
| | —CH₂—S—C(CH₃)₂— | | C(O)O-]benzyl- | |
| | (L-5,5-dimethylthiazolidin-4-yl) | | | |
| p-CH₃-φ- | R²/R³ = cyclic | | p-[HC(O)OCH₂CH₂N(CH₃)C(O)O-]benzyl- | —OH |
| | 3 carbon atoms | | | |
| | (L-pyrrolidinyl) | | | |
| p-CH₃-φ- | R²/R³ = cyclic | | p-[HOCH₂CH₂N(CH₃)C(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[CH₃OC(O)CH₂NHC(O)O-]benzyl- | —OC(CH₃)₃ |
| | 3 carbon atoms | | | |
| | (L-pyrrolidinyl) | | | |
| quinolin-8-yl- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| 3,4-difluoro-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(thiomorpholin-4-yl)-C(O)O-]benzyl- | —OCH(CH₃)₂ |
| | 3 carbon atoms | | | |
| | (L-pyrrolidinyl) | | | |
| p-CH₃O-φ- | R²/R³ = cyclic | | p-[(CH₃)₂NC(O)O-]benzyl- | —OH |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OH |
| | —CH₂—CH₂—(O)—CH₂— | | | |
| | (L-1-oxothiomorpholin-4-yl) | | | |
| 3,4-difluoro-φ- | R²/R³ = cyclic | | p-[(CH₃)₂NC(O)O-]benzyl- | —OC(CH₃)₃ |
| | —CH₂CH₂—SO₂—CH₂— | | | |
| | (L-1,1-dioxothiomorpholin-3-yl) | | | |
| p-H₂N-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| 3,4-difluoro-φ- | R²/R³ = cyclic | | p-[(CH₃)₂NC(O)O-]benzyl- | —OH |
| | —CH₂CH₂—SO₂—CH₂— | | | |
| | (L-1,1-dioxothiomorpholin-3-yl) | | | |
| 3,4-difluoro-φ- | R²/R³ = cyclic | | p-[(CH₃)₂NC(O)O-]benzyl- | —OH |
| | 3 carbon atoms | | | |
| | (L-pyrrolidinyl) | | | |
| quinolin-8-yl- | R²/R³ = cyclic | | p-[(CH₃)₂NC(O)O-]benzyl- | —OH |
| | 3 carbon atoms | | | |
| | (L-pyrrolidinyl) | | | |
| 1-methyl- | R²/R³ = cyclic | | p-[(CH₃)₂NC(O)O-]benzyl- | —OCH(CH₃)₂ |
| pyrazol-4-yl- | —CH₂—S—C(CH₃)₂— | | | |
| | (L-5,5-dimethylthiazolidin-4-yl) | | | |
| p-CH₃-φ- | R²/R³ = cyclic | | p-[(CH₃)₂NC(O)O-]benzyl- | —OC(CH₃)₃ |
| | —CH₂—CH₂—(O)—CH₂— | | | |
| | (L-1-oxothiomorpholin-4-yl) | | | |
| 1-n-butyl- | R²/R³ = cyclic | | p-[(CH₃)₂NC(O)O-]benzyl- | —OCH(CH₃)₂ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH(CH₃)₂ |
| pyrazol-3-yl | —CH₂—S—C(CH₃)₂— | | | |
| | (L-5,5-dimethylthiazolidin-4-yl) | | | |
| 2-(CF₃C(O)-)- | R²/R³ = cyclic | | p-[(CH₃)₂NC(O)O-]benzyl- | —OCH(CH₃)₂ |
| 1,2,3,4- | 3 carbon atoms | | | |
| tetrahydro- | (L-pyrrolidinyl) | | | |
| isoquinolin-7-yl | | | | |
| p-CH₃-φ- | R²/R³ = cyclic | | p-[(4-(φNHC(O)-)-piperazin-1-yl)- | —OH |
| | 3 carbon atoms | | C(O)O-]benzyl- | |
| | (L-pyrrolidinyl) | | | |
| p-CH₃-φ- | R²/R³ = cyclic | | p-[(4-methoxypiperidin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(4-(pyridin-4-ylC(O))piperazin-1-yl)- | —OCH(CH₃)₂ |
| | 3 carbon atoms | | C(O)O-]benzyl- | |
| | (L-pyrrolidinyl) | | | |
| p-CH₃-φ- | R²/R³ = cyclic | | p-[(CH₃)₂NC(O)O-]benzyl- | —OC(CH₃)₃ |
| | —CH₂C(O)-—CH₂— | | | |
| | (L-4-oxopyrrolidinyl) | | | |
| p-CH₃-φ- | R²/R³ = cyclic | | p-[(CH₃)₂NC(O)O-]benzyl- | —OC(CH₃)₃ |
| | —CH₂—CH(OH)CH₂— | | | |
| | (L-4-hydroxopyrrolidinyl) | | | |
| m-F-φ- | R²/R³ = cyclic | | p-[(CH₃)₂NC(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(4-methoxypiperidin-1-yl)C(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(4-φNHC(O)-)piperazin-1-yl)-C(O)O-]benzyl- | —OCH(CH₃)₂ |
| | 3 carbon atoms | | | |
| | (L-pyrrolidinyl) | | | |
| p- | R²/R³ = cyclic | | p-[(CH₃)₂NC(O)O-]benzyl- | —OC(CH₃)₃ |
| (φNHC(O)NH)φ- | 3 carbon atoms | | | |
| | (L-pyrrolidinyl) | | | |
| p-F-φ- | R²/R³ = cyclic | | 3-chloro-4-[(4-methylpiperidin-1-yl)-C(O)O-]- | —OCH(CH₃)₂ |
| | —CH₂—S—C(CH₃)₂— | | benzyl- | |
| | (L-5,5-dimethylthiazolidin-4-yl) | | | |
| p-CH₃-φ- | R²/R³ = cyclic | | 3-chloro-4-[(CH₃)₂NC(O)O-]-benzyl- | —OCH(CH₃)₂ |
| | —CH₂—S—C(CH₃)₂— | | | |
| | (L-5,5-dimethylthiazolidin-4-yl) | | | |
| p-CH₃-φ- | R²/R³ = cyclic | | p-[(4-(CH₃SO₂-)piperazin-1-yl)-C(O)O]benzyl- | —OC(CH₃)₃ |
| | 3 carbon atoms | | | |
| | (L-pyrrolidinyl) | | | |
| p-CH₃-φ- | R²/R³ = cyclic | | p-[(morpholin-4-yl)CH₂CH₂NHC(O)O-]benzyl- | —OH |
| | 3 carbon atoms | | | |
| | (L-pyrrolidinyl) | | | |
| p-CH₃-φ- | R²/R³ = cyclic | | p-[(4-(HO(O)-)piperidin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(HOCH₂CH2)₂NC(O)O-]benzyl- | —OCH(CH₃)₂ |
| | 3 carbon atoms | | | |
| | (L-pyrrolidinyl) | | | |
| p-O₂N-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(4-(HOCH₂-)piperidin-1-yl)-C(O)O-]benzyl- | —OCH(CH₃)₂ |
| | 3 carbon atoms | | | |
| | (L-pyrrolidinyl) | | | |
| p-CH₃-φ- | R²/R³ = cyclic | | 3-chloro-4-[(CH₃)₂NC(O)O)-]-benzyl- | —OCH(CH₃)₂ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OCH(CH₃)₂ |
| | —CH₂—CH₂—S—CH₂— | | | |
| | (L-thiomorpholin-3-yl) | | | |
| 1-methyl- | R²/R³ = cyclic | | p-[(CH₃)₂NC(O)O-]benzyl- | —OH |
| pyrazol-3-yl- | —CH₂—S—C(CH₃)₂— | | | |
| | (L-5,5-dimethylthiazolidin-4-yl) | | | |
| m-F-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| o-F-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 3,4-difluoro-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 3,5-difluoro-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 2,4-difluoro-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-NH₂-φ- | R²/R³ = cyclic | | p-[(CH₃)₂NC(O)O-]benzyl- | —OH |
| | 3 carbon atoms | | | |
| | (L-pyrrolidinyl) | | | |
| p-N≡C-φ- | R²/R³ = cyclic | | p-[(CH₃)₂NC(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OH |
| | —CH₂CH(OH)CH₂— | | | |
| | (L-4-hydroxypyrrolidinyl) | | | |
| p-CH₃-φ- | R²/R³ = cyclic | | p-[(CH₃)₂NC(O)O-]benzyl- | —OH |
| | —CH₂C(O)CH₂— | | | |
| | (L-4-oxypyrrolidinyl) | | | |
| pyridin-2-yl- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-Cl-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| m-Cl-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| o-Cl-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 3,4-dichloro-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 3,4-dichloro-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂CH₃ |
| pyrazol-4-yl- | —CH₂—S—C(CH₃)₂— | | | |
| | (L-5,5-dimethylthiazolidin-4-yl) | | | |
| pyridin-3-yl- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| 1-methyl- | R²/R³ = cyclic | | p-[(thiomorpholin-4-yl)-C(O)O-]benzyl- | —OCH(CH₃)₂ |
| pyrazol-4-yl- | —CH₂—S—C(CH₃)₂— | | | |
| | (L-5,5-dimethylthiazolidin-4-yl) | | | |
| quinolin-8-yl- | R²/R³ = cyclic | | p-[(CH₃)₂NC(O)O-]benzyl- | —OCH(CH₃)₂ |
| | —CH₂—S—C(CH₃)— | | | |
| | (L-5,5-dimethylthiazolidin-4-yl) | | | |
| m-Cl-φ- | R²/R³ = cyclic | | p-[(CH₃)₂NC(O)O-]benzyl- | —OC(CH₃)₃ |
| | —CH₂CH₂—SO₂—CH₂— | | | |
| | (L-1,1-dioxothiomorpholin-3-yl) | | | |
| pyridin-2-yl | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| 3,4-dichloro-φ- | R²/R³ = cyclic | | p-[(CH₃)₂NC(O)O-]benzyl- | —OC(CH₃)₃ |
| | —CH₂CH₂—SO₂—CH₂— | | | |
| | (L-1,1-dioxothiomorpholin-3-yl) | | | |
| 2,5-dichloro- | R²/R³ = cyclic | | p-[(CH₃)₂NC(O)O-]benzyl- | —OCH(CH₃)₂ |
| thien-3-yl- | —CH₂—S—C(CH₃)₂— | | | |
| | (L-5,5-dimethylthiazolidin-4-yl) | | | |
| p-CH₃O-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| m-CH₃O-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| o-CH₃O-φ- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| 3,4-dimethoxy-φ- | R²/R³ = cyclic | | p-[(CH₃)₂NC(O)O-]benzyl- | —OCH(CH₃)₂ |
| | —CH₂—S—C(CH₃)₂ | | | |
| | (L-5,5-dimethylthiazolidin-4-yl) | | | |
| 2,4-difluoro-φ- | R²/R³ = cyclic | | p-[(CH₃)₂NC(O)O-]benzyl- | —OC(CH₃)₃ |
| | —CH₂—CH₂—S—CH₂— | | | |
| | (L-thiomorpholin-3-yl) | | | |
| 3,4-dichloro-φ- | R²/R³ = cyclic | | p-[(CH₃)₂NC(O)O-]benzyl- | —OH |
| | —CH₂CH₂—SO₂—CH₂— | | | |
| | (L-1,1-dioxothiomorpholin-3-yl) | | | |
| m-Cl-φ- | R²/R³ = cyclic | | p-[(CH₃)₂NC(O)O-]benzyl- | —OH |
| | —CH₂CH₂—SO₂—CH₂— | | | |
| | (L-1,1-dioxothiomorpholin-3-yl) | | | |
| 2,4-difluoro-φ- | R²/R³ = cyclic | | p-[(CH₃)₂NC(O)O-]benzyl- | —OC(CH₃)₃ |
| | —CH₂—CH₂—S(O)—CH₂— | | | |
| | (L-1-oxothiomorpholin-4-yl) | | | |
| p-CH₃-φ- | R²/R³ = cyclic | | p-[(4-methylpiperzin-1-yl)C(O)O-]benzyl- | —OCH(CH₃)₂ |
| | —CH₂—C(O)—CH₂— | | | |
| | (L-4-oxopyrrolidinyl) | | | |
| p-CH₃-φ- | R²/R³ = cyclic | | p-[(thiomorpholin-4y1)-C(O)O-]benzyl- | —OC(CH₃)₃ |
| | —CH₂CH(OH)CH₂— | | | |
| | (L-4-hydroxypyrrolidinyl) | | | |
| p-CH₃-φ- | R²/R³ = cyclic | | p-[(3-(HOCH2-)piperidin-1-yl)-C(O)O-]benzyl- | —OH |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OCH(CH₃)₂ |
| | —CH₂—CF₂—CH₂— | | | |
| | (L-4,4-difluoro-pyrrolidinyl) | | | |
| p-CH₃-φ- | R²/R³ = cyclic | | p-[(CH₃)₂NC(O)O-]benzyl- | —O(CH₂CH₂O)₂CH₃ |
| | 3 carbon atoms | | | |
| | (L-pyrrolidinyl) | | | |
| p-CH₃-φ- | R²/R³ = cyclic | | p-[(thiomorpholin-4-yl)-C(O)O-]benzyl- | —OC(CH₃)₃ |
| | —CH₂CH(—O— | | | |
| | C(O)thiomorpholin-4-yl-CH₂— | | | |
| | (L-4-(thiomorpholin-4- | | | |
| | yl)C(O)O-pyrrolidinyl) | | | |
| p-CH₃-φ- | R²/R³ = cyclic | | p-[(CH₃)₂NC(O)O-]benzyl- | —OH |
| | —CH₂—CF₂—CH₂— | | | |
| | (L-4,4-difluoro-pyrolidinyl) | | | |
| p-F-φ- | R²/R³ = cyclic | | p-[(4-(pyrimidin-2-yl)piperazin-1-yl)- | —OC(CH₃)₃ |
| | —CH₂—S—C(CH₃)₂— | | C(O)O-]benzyl- | |
| | (L-5,5-dimethylthiazolidin-4-yl) | | | |
| p-CH₃-φ- | R²/R³ = cyclic | | p-[(4-(φC(O)-)piperazin-1-yl)-C(O)O-]benzyl- | —OCH(CH₃)₂ |
| | 3 carbons atoms | | | |
| | (L-pyrrolidinyl) | | | |
| p-F-φ- | R²/R³ = cyclic | | 3-fluoro-4-[(CH₃)₂NC(O)O-]-benzyl- | —OCH(CH₃)₂ |
| | —CH₂—S—C(CH₃)₂— | | | |
| | (L-5,5-dimethylthiazolidin-4-yl) | | | |
| p-F-φ- | R²/R³ = cyclic | | 3-chloro-4-[(4-(pyridin-2-yl)piperazin-1- | —OCH(CH₃)₂ |
| | —CH₂—S—C(CH₃)₂— | | yl)C(O)O-]-benzyl- | |
| | (L-5,5-dimethylthiazolidin-4-yl) | | | |
| p-F-φ- | R²/R³ = cyclic | | 3-chloro-4-[(4-(pyridin-2-yl)piperazin-1- | —OC(CH₃)₃ |
| | —CH₂—S—C(CH₃)₂— | | yl)C(O)O-]-benzyl- | |
| | (L-5,5-dimethylthiazolidin-4-yl) | | | |
| p-CH₃-φ- | R²/R³ = cyclic | | p-[(CH₃)₂NC(O)O-]benzyl- | —OC(CH₃)₃ |
| | —CH₂CH₂N— | | | |
| | (—SO₂CH₃)—CH₂— | | | |
| | (L-4-methanesulfonyl- | | | |
| | piperazinyl) | | | |
| 1-methyl- | R²/R³ = cyclic | | p-[(CH₃)₂NC(O)O-]benzyl- | —OCH(CH₃)₂ |
| imidazol-4-yl- | 3 carbon atoms | | | |
| | (L-pyrrolidinyl) | | | |
| p-Br-φ- | R²/R³ = cyclic | | p-[(CH₃)₂NC(O)O-]benzyl- | —OC(CH₃)₃ |
| | —CH₂—S—C(CH₃)₂— | | | |
| | (L-5,5-dimethylthiazolidin-4-yl) | | | |
| p-Br-φ- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| p-NH₂C(═N)-φ- | R²/R³ = cyclic | | p-[(CH₃)₂NC(O)O-]benzyl- | —OCH₃ |
| | 3 carbon atoms | | | |
| | (L-pyrrolidinyl) | | | |
| p-N≡C-φ- | R²/R³ = cyclic | | p-[(thiomorpholin-4-yl)-C(O)O-]benzyl- | —OCH(CH₃)₂ |
| | 3 carbon atoms | | | |
| | (L-pyrrolidinyl) | | | |
| p-CH₃-φ | R²/R³ = cyclic | | p-[(thiomorpholin-4-yl)-C(O)O-]benzyl- | —OH |
| | —CH₂CH(—O- | | | |
| | C(O)thiomorpholin-4-yl)-CH₂— | | | |
| | (L-4-(thiomorpholin-4- | | | |
| | yl)C(O)O-pyrrolidinyl) | | | |
| p-CH₃-φ- | R²/R³ = cyclic | | p-[(thiomorpholin-4-yl)-C(O)O-]benzyl- | —OC(CH₃)₃ |
| | —CH₂—C(O)—CH₂— | | | |
| | (L-4-oxopyrrolidinyl) | | | |
| p-CH₃-φ- | R²/R³ = cyclic | | p-[(thiomorpholin-4-yl)-C(O)O-]benzyl- | —OH |
| | —CH₂—C(O)—CH₂— | | | |
| | (L-4-oxopyrrolidinyl) | | | |
| p-CH₃-φ- | R²/R³ = cyclic | | p-[(4-methylpiperazin-1-yl)-C(O)O-]benzyl- | —OH |
| | —CH₂—C(O)—CH₂— | | | |
| | (L-4-oxopyrrolidinyl) | | | |
| p-F-φ- | R²/R³ = cyclic | | p-[(4-(pyrimidin-2-yl)piperazin-1-yl)- | —OH |
| | —CH₂—S—C(CH₃)₂— | | C(O)O-]benzyl- | |
| | (L-5,5-dimethylthiazolidin-4-yl) | | | |
| quinolin-8-yl- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| pyridin-4-yl- | R²/R³ = cyclic | | p-[(CH₃)₂NC(O)O-]benzyl- | —OCH(CH₃)₂ |
| | —CH₂—S—C(CH₃)₂— | | | |
| | (L-5,5-dimethylthiazolidin-4-yl) | | | |
| p-F-φ- | R²/R³ = cyclic | | p-[(4-(pyridin-2-yl)piperazin-1-yl)C(O)O-]benzyl- | —OCH(CH₃)₂ |
| | —CH₂—S—C(CH₃)₂— | | | |
| | (L-5,5-dimethylthiazolidin-4-yl) | | | |
| m-F-φ- | R²/R³ = cyclic | | p-[(CH₃)₂NC(O)O-]benzyl- | —OH |
| | 3 carbon atoms | | | |
| | (L-pyrrolidinyl) | | | |
| p-F-φ- | R²/R³ = cyclic | | p-[(CH₃)₂NC(O)O-]benzyl- | —OH |
| | —CH₂—CH₂—S— | | | |
| | (thiazolidin-2-yl) | | | |
| p-F-φ- | R²/R³ = cyclic | | p-[(CH₃)₂NC(O)O-]benzyl- | —OC(CH₃)₃ |
| | —CH₂—CH₂—S— | | | |
| | (thiazolidin-2-yl) | | | |
| p-CH₃-φ- | R²/R³ = cyclic | | p-[(thiomorpholin-4-yl)C(O)O-]benzyl- | —OH |
| | —CH₂—C(O)—CH₂— | | | |
| | (L-4-oxopyrrolidinyl) | | | |
| p-NH₂- | R²/R³ = cyclic | | p-[(thiomorpholin-4-yl)C(O)O-]benzyl- | —OCH₃ |
| C(═N)-φ- | 3 carbon atoms | | | |
| | (L-pyrrolidinyl) | | | |
| p-CH₃-φ- | R²/R³ = cyclic | | p-[(4-methylpiperazin-1-yl)-C(O)O-]benzyl- | —OH |
| | —CH₂—C(O)—CH₂— | | | |
| | (L-4-oxopyrrolidinyl) | | | |
| p-F-φ- | R²/R³ = cyclic | | p-[(4-pyridin-2-yl)piperazin-1-yl)-C(O)O-]benzyl- | —OH |
| | —CH₂—CH₂—S— | | | |
| | (thiazolidin-2-yl) | | | |
| p-NO₂-φ- | ²/R³ = cyclic | | p-[(4-pyridin-2-yl)piperazin-1-yl)-C(O)O-]benzyl- | —OC(CH₃)₃ |
| | 3 carbon atoms | | | |
| | (L-pyrrolidinyl) | | | |
| p-F-φ- | R²/R³ = cyclic | | p-[(4-pyridin-2-yl)piperazin-1-yl)-C(O)O-]benzyl- | —OC(CH₃)₃ |
| | —CH₂—CH₂—S— | | | |
| | (thiazolidin-2-yl) | | | |
| p-Br-φ- | R²/R³ = cyclic | | p-[(4-pyridin-2-yl)piperazin-1-yl)-C(O)O-]benzyl- | —OH |
| | —CH₂—S—C(CH₃)₂— | | | |
| | (L-5,5-dimethylthiazolidin-4-yl) | | | |
| p-CH₃-φ- | R²/R³ = cyclic | | p-[(4-(qC(O)-)piperazin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(4-(φNHC(S)-)piperazin-1-yl)C(O)O-]benzyl- | —OCH(CH₃)₂ |
| | 3 carbon atoms | | | |
| | (L-pyrrolidinyl) | | | |
| p-F-φ- | R²/R³ = cyclic | | p-[(4-CH₃-homopiperazin-1-yl)C(O)O-]benzyl- | —OC(CH₃)₃ |
| | —CH₂—CH₂—S— | | | |
| | (thiazolidin-2-yl) | | | |
| p-CH₃-φ- | R²/R³ = cyclic | | p-[p-CH₃-φ-SO₂N(CH₃)CH₂CH₂N(CH₃)— | —OCH(CH₃)₂ |
| | 3 carbon atoms | | C(O)O-]benzyl- | |
| | (L-pyrrolidinyl) | | | |
| p-CH₃-φ- | R²/R³ = cyclic | | p-[φNHC(O)O-CH₂CH₂NHC(O)O-]benzyl- | —OCH(CH₃)₂ |
| | 3 carbon atoms | | | |
| | (L-pyrrolidinyl) | | | |
| 3-Cl-4-F-φ- | R²/R³ = cyclic | | p-[(CH₃)₂NC(O)O-]benzyl- | —OC(CH₃)₃ |
| | —CH₂CH₂—SO₂—CH₂— | | | |
| | (L-1,1-dioxothiomorpholin-3-yl) | | | |
| 1-methyl- | R²/R³ = cyclic | | p-[(CH₃)₂NC(O)O-]benzyl- | —OC(CH₃)₃ |
| pyraol-4-yl- | —CH₂CH₂—S—CH₂— | | | |
| | (L-thiomorpholin-3-yl) | | | |
| p-CH₃-φ- | R²/R³ = cyclic | | p-[(CH₃)₂NC(O)O-]benzyl- | —OC(CH₃)₃ |
| | —CH₂CH(—OSO₂CH₃)—CH₂— | | | |
| | (L-4-methanesulfoxy- | | | |
| | pyrrolidinyl) | | | |
| p-H₂NC(O)-φ- | R²/R³ = cyclic | | p-[(CH₃)₂NC(O)O-]benzyl- | —OH |
| | 3 carbon atoms | | | |
| | (L-pyrrolidinyl) | | | |
| p-H₂N-C(═N)-φ | R²/R³ = cyclic | | p-[(CH₃)₂NC(O)O-]benzyl- | —OH |
| | 3 carbon atoms | | | |
| | (L-pyrrolidinyl) | | | |
| p-H₂NC(O)-φ- | R²/R³ = cyclic | | p-[(thiomorpholin-4-yl)C(O)O-]benzyl- | —OH |
| | 3 carbon atoms | | | |
| | (L-pyrrolidinyl) | | | |
| p- | R²/R³ = cyclic | | p-[(thiomorpholin-4-yl)C(O)O-]benzyl- | —OH |
| H₂N—C(═N)-φ | 3 carbon atoms | | | |
| | (L-pyrrolidinyl) | | | |
| p-NO₂-φ- | R²/R³ = cyclic | | p-[(4-(pyridin-2-yl)piperazin-1-yl)- | —OH |
| | 3 carbon atoms | | C(O)O-]benzyl- | |
| | (L-pyrrolidinyl) | | | |
| p-F-φ- | R²/R³ = cyclic | | 3-chloro-4-[(4-(pyridin-2-yl)piperazin-1- | —OCH₂CH₃ |
| | —CH₂—S—C(CH₃)₂— | | ylC(O)O-]-benzyl- | |
| | (L-5,5-dimethylthiazolidin-4-yl) | | | |
| p-F-φ- | R²/R³ = cyclic | | 3-chloro-4-[(4-(pyridin-2-yl)piperazin-1- | —OH |
| | —CH₂—S—C(CH₃)₂— | | yl)C(O)O-]-benzyl- | |
| | (L-5,5-dimethylthiazolidin-4-yl) | | | |
| p-F-φ- | R²/R³ = cyclic | | p-[(4-CH₃-homopiperazin-1-yl)C(O)O-]-benzyl- | —OH |
| | —CH₂—CH₂—S— | | | |
| | (thiazolidin-2-yl) | | | |
| 1-methyl- | R²/R³ = cyclic | | 3-chloro-4-[(CH₃)₂NC(O)O-]benzyl- | —OCH(CH₃)₂ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(4-(pyridin-2-yl)piperazin-1-yl)C(O)O-]benzyl- | —OCH(CH₃)₂ |
| imidazol-4-yl- | 3 carbon atoms | | | |
| | (L-pyrrolidinyl) | | | |
| 1-methyl- | R²/R³ = cyclic | | p-[(4-(pyridin-2-yl)piperazin-1-yl)C(O)O-]benzyl- | —OC(CH₃)₃ |
| imidazol-4-yl- | 3 carbon atoms | | | |
| | (L-pyrrolidinyl) | | | |
| p-CH₃-φ- | R²/R³ = cyclic | | p-[(4-(pyridin-2-yl)piperazin-1-yl)C(O)O-]benzyl- | —OH |
| | 3 carbon atoms | | | |
| | (L-pyrrolidinyl) | | | |
| p-CH₃-φ- | R²/R³ = cyclic | | p-[(4-(pyridin-2-yl)piperazin-1-yl)C(O)O-]benzyl- | —OC(CH₃)₃ |
| | 3 carbon atoms | | | |
| | (L-pyrrolidinyl) | | | |
| p-CH₃-φ- | R²/R³ = cyclic | | p-[(4-(pyridin-2-yl)piperazin-1-yl)C(O)O-]benzyl- | —OCH(CH₃)₂ |
| | 3 carbon atoms | | | |
| | (L-pyrrolidinyl) | | | |
| p-F-φ- | R²/R³ = cyclic | | p-[(4-(pyridin-2-yl)piperazin-1-yl)C(O)O-]benzyl- | —OCH(CH₃)₂ |
| | 3 carbon atoms | | | |
| | (L-pyrrolidinyl) | | | |
| p-F-φ- | R²/R³ = cyclic | | p-[(4-(pyridin-2-yl)piperazin-1-yl)C(O)O-]benzyl- | —OCH(CH₃)₂ |
| | 3 carbon atoms | | | |
| | (L-pyrrolidinyl) | | | |
| p-CH₃-φ- | R²/R³ = cyclic | | p-[(CH₃)₂NC(O)O-]benzyl- | —OH |
| | —CH₂CH₂N(—SO₂—CH₃)₂— | | | |
| | (4-methanesulfonyl-piperazin-2- | | | |
| | yl) | | | |
| p-CH₃-φ- | R²/R³ = cyclic | | p-[(CH₃)₂NC(O)O-]benzyl- | —OH |
| | —CH₂CH(—OSO₂—CH₃)CH₂— | | | |
| | (L-4-methanesulfoxy- | | | |
| | pyrrolidinyl) | | | |
| 3,4-difluoro-φ- | R²/R³ = cyclic | | p-[(CH₃)₂NC(O)O-]benzyl- | —OC(CH₃)₃ |
| | —CH₂—CH₂—S—CH₂— | | | |
| | (L-thiomorpholin-3-yl) | | | |
| pyridin-3-yl | R²/R³ = cyclic | | p-[(thiomorpholin-4-yl)C(O)O-]benzyl- | —OCH(CH₃)₂ |
| | —CH₂—S—C(CH₃)₂— | | | |
| | (L-5,5-dimethylthiazolidin-4-yl) | | | |
| 3,4-difluoro-φ- | R²/R³ = cyclic | | p-[(CH₃)₂NC(O)O-]benzyl- | —OH |
| | —CH₂—CH₂—S—CH₂— | | | |
| | (L-thiomorpholin-3-yl) | | | |
| p-F-φ- | R²/R³ = cyclic | | p-[(CH₃)₂NC(O)O-]benzyl- | —OCH(CH₃)₂ |
| | —CH₂CH(OH)CH₂— | | | |
| | (L-4-hydroxypyrrolidinyl) | | | |
| p-Br-φ- | R²/R³ = cyclic | | p-[(4-(pyridin-2-yl)piperazin-1-yl)C(O)O-]benzyl- | —OC(CH₃)₃ |
| | —CH₂—S—C(CH₃)₂— | | | |
| | (L-5,5-dimethylthiazolidin-4-yl) | | | |
| p-CF₃O-φ- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| p-CF₃O-φ- | R²/R³ = cyclic | | p-[(CH₃)₂NC(O)O-]benzyl- | —OC(CH₃)₃ |
| | —CH₂—S—C(CH₃)₂— | | | |
| | (L-5,5-dimethylthiazolidin-4-yl) | | | |
| p-CF₃O-φ- | R²/R³ = cyclic | | p-[(4-(pyridin-2-yl)piperazin-1-yl)C(O)O-]benzyl- | —OC(CH₃)₃ |
| | —CH₂—S—C(CH₃)₂— | | | |
| | (L-5,5-dimethylthiazolidin-4-yl) | | | |
| p-F-φ- | R²/R³ = cyclic | | p-[(4-(pyridin-2-yl)piperazin-1-yl)C(O)O-]benzyl- | —OH |
| | 3 carbon atoms | | | |
| | (L-pyrrolidinyl) | | | |
| p-F-φ- | R²/R³ = cyclic | | p-[(4-(pyridin-2-yl)piperazin-1-yl)C(O)O-]benzyl- | —OH |
| | —CH₂CH(OH)CH₂— | | | |
| | (L-4-hydroxypyrrolidinyl) | | | |
| p-CF₃O-φ- | R²/R³ = cyclic | | p-[(4-(pyridin-2-yl)piperazin-1-yl)C(O)O-]benzyl- | —OH |
| | —CH₂—S—C(CH₃)₂— | | | |
| | (L-5,5-dimethylthiazolidin-4-yl) | | | |
| 1-methyl- | R²/R³ = cyclic | | 3-chloro-4-[(CH₃)₂NC(O)O)-]benzyl- | —OH |
| imidazol-4-yl- | 3 carbon atoms | | | |
| | (L-pyrrolidinyl) | | | |
| 1-methyl- | R²/R³ = cyclic | | 3-chloro-4-[(CH₃)₂NC(O)O)-]benzyl- | —OCH(CH₃)₂ |
| imidazol-4-yl- | 3 carbon atoms | | | |
| | (L-pyrrolidinyl) | | | |
| 1-methyl- | R²/R³ = cyclic | | p-[(4-(pyridin-2-yl)piperazin-1-yl)C(O)O-]benzyl- | —OH |
| imidazol-4-yl- | 3 carbon atoms | | | |
| | (L-pyrrolidinyl) | | | |
| 1-methyl- | R²/R³ = cyclic | | p-[(4-(pyridin-2-yl)piperazin-1-yl)C(O)O-]benzyl- | —OH |
| imidazol-4-yl- | —CH₂—S—C(CH₃)₂— | | | |
| | (L-5,5-dimethylthiazolidin-4-yl) | | | |
| 1-methyl- | R²/R³ = cyclic | | p-[(4-(pyridin-2-yl)piperazin-1-yl)C(O)O-]benzyl- | —OH |
| pyrazol-4-yl- | 3 carbon atoms | | | |
| | py(L-pyrrolidinyl) | | | |
| 1-methyl- | R²/R³ = cyclic | | p-[(4-(pyridin-2-yl)piperazin-1-yl)C(O)O-]benzyl- | —OCH(CH₃)₂ |
| pyrazol-4-yl- | 3 carbon atoms | | | |
| | (L-pyrrolidinyl) | | | |
| 1-methyl- | R²/R³ = cyclic | | p-[(4-(pyridin-2-yl)piperazin-1-yl)C(O)O-]benzyl- | —OC(CH₃)₃ |
| pyrazol-4-yl- | 3 carbon atoms | | | |
| | (L-pyrrolidinyl) | | | |
| 1-methyl- | R²/R³ = cyclic | | p-[(4-(pyridin-2-yl)piperazin-1-yl)C(O)O-]benzyl- | —OC(CH₃)₃ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | 3-chloro-4-[(4-(pyridin-2-yl)piperazin-1- | —OCH(CH₃)₂ |
| imidazol-4-yl- | 3 carbon atoms | | yl)C(O)O]benzyl- | |
| | (L-pyrrolidinyl) | | | |
| 1-methyl- | R²/R³ = cyclic | | p-[(CH₃)₂NC(O)O-]benzyl- | —OCH₂CH₂Oφ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | 3-chloro-4-[(4-(pyridin-2-yl)piperazin-1- | —OH |
| | —CH₂—S—C(CH₃)₂— | | yl)C(O)O-]-benzyl- | |
| | (L-5,5-dimethylthiazolidin-4-yl) | | | |
| 1-methyl- | R²/R³ = cyclic | | 3-chloro-4-[(4-(pyridin-2-yl)piperazin-1- | —OCH₂CH₃ |
| pyrazol-4-yl- | —CH₂—S—C(CH₃)₂— | | yl)C(O)O-]-benzyl- | |
| | (L-5,5-dimethylthiazolidin-4-yl) | | | |
| 1,5-dimethyl- | R²/R³ = cyclic | | p-[4-[5-CF₃-pyridin-2-yl)piperazin-1 yl)- | —OH |
| 3-chloro- | —CH₂—S—C(CH₃)₂— | | C(O)O-]benzyl- | |
| pyrazol-4-yl- | (L-5,5-dimethylthiazolidin-4-yl) | | | |
| p-F-φ- | R²/R³ = cyclic | | p-[(CH₃)₂NC(O)O-]benzyl- | —OC(CH₃)₃ |
| | —CH₂CH(OH)CH₂— | | | |
| | (L-4-hydroxypyrrolidinyl) | | | |
| pyridin-4-yl- | R²/R³ = cyclic | | p-[(CH₃)₂NC(O)O-]benzyl- | —OH |
| | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂C(CH₃)₃ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂O— |
| pyrazol-4-yl | —CH₂—S—C(CH₃)₂— | | | C(O)C(C(CH₃)₃ |
| | (L-5,5-dimethylthiazolidin-4-yl) | | | |
| pyridin-3-yl- | R²/R³ = cyclic | | p-[(CH₃)₂NC(O)O-]benzyl- | —OCH₂C(CH₃)₃ |
| | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | 2-CH₃O-φ-O- |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂cyclopropyl |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂CH₂CH₃ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —OCH₂CH₂CH₂CH₃ |
| pyrazol-4-yl- | —CH₂—S—C(CH₃)₂— | | | |
| | (L-5,5-dimethylthiazolidin-4-yl) | | | |
| 1-methyl- | R²/R³ = cyclic | | p-[(CH₃)₂NC(O)O-]benzyl- | —O—CH₃ |
| pyrazol-4-yl- | —CH₂—S—C(CH₃)₂— | | | |
| | (L-5,5-dimethylthiazolidin-4-yl) | | | |
| pyridin-3-yl- | R²/R³ = cyclic | | p-[(CH₃)₂NC(O)O-]benzyl- | —OC(CH₃)₃ |
| | —CH₂—S—C(CH₃)₂— | | | |
| | (L-5,5-dimethylthiazolidin-4-yl) | | | |
| pyridin-3-yl | R²/R³ = cyclic | | p-[(CH₃)₂NC(O)O-]benzyl- | —OCH₂CH₃ |
| | —CH₂—S—C(CH₃)₂— | | | |
| | (L-5,5-dimethylthiazolidin-4-yl) | | | |
| pyridin-3-yl- | R²/R³ = cyclic | | p-[(CH₃)₂NC(O)O-]benzyl- | —OCH₂cyclopropyl |
| | —CH₂—S—C(CH₃)₂— | | | |
| | (L-5,5-dimethylthiazolidin-4-yl) | | | |

In a preferred embodiment, the compounds are defined by formula Ia below 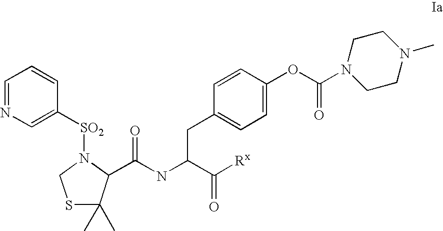
wherein R^{x} is hydroxy or C₁₋₅ alkoxy and pharmaceutically acceptable salts thereof. Preferably, the compound is N-[N-(3-pyridinesulfonyl)-L-3,3-dimethyl-4-thiaprolyl]-O-[1-methylpiperazin-4-ylcarbonyl]-L-tyrosine isopropyl ester.

In another aspect the compounds that can be utilized as steroid sparing agents are compounds defined by formula II below: 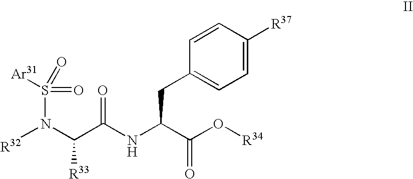
wherein:
Ar³¹ is selected from the group consisting of aryl, substituted aryl, heteroaryl, and substituted heteroaryl;
R³² and R³³ together with the nitrogen atom bound to R³² and the carbon atom bound to R³³ form a heterocylic or substituted heterocylic group.
R³⁴ is selected from the group consisting of hydrogen, alkyl, substituted alkyl, cycloalkyl, substituted cycloalkyl, aryl, and substituted aryl; and
R³⁷ is aryl, heteroaryl, substituted aryl, substituted heteroaryl, heterocyclic, substituted heterocyclic, aryloxy, substituted aryloxy, aralkoxy, substituted aralkoxy, heteroaryloxy, substituted heteroaryloxy;
and pharmaceutically acceptable salts thereof.

In another preferred embodiment, R³² and R³³, in the compounds of formula II, together with the nitrogen atom bound to R³² and the carbon atom bound to R³³ form a saturated heterocyclic group or a saturated substituted heterocyclic group with the proviso that when monosubstituted, the substituent on said saturated substituted heterocyclic group is not carboxyl.

Preferably, in the compounds of formula II above, R³² is alkyl, substituted alkyl, or R³² and R³³ together with the nitrogen atom bound to R³² and the carbon atom bound to R³³ form a heterocyclic or substituted heterocyclic group and R³⁴ is hydrogen or alkyl.

Preferably, in the compounds of formula II above, R³⁷ is aryl, substituted aryl, heteroaryl, substituted heteroaryl, heterocyclic, or substituted heterocyclic. In a preferred embodiment, R³⁷ is substituted aryl wherein the aryl is substituted with one to three substituents independently selected from the group consisting alkyl and alkoxy. In a preferred embodiment, R³⁷ is substituted heteroaryl wherein the heteroaryl is substituted with one to three substituents independently selected from the group consisting alkyl, alkoxy, and oxo. In another preferred embodiment R³⁷ is substituted aryl or heteroaryl wherein aryl or heteroaryl is 2,6-di-substituted. In yet another preferred embodiment R³⁷ is 2,6-di-substituted aryl wherein the substituents are independently selected from the group consisting of alkyl and alkoxy. In yet another preferred embodiment R³⁷ is 2,6-di-substituted heteroaryl wherein the substituents are independently selected from the group consisting of alkyl, oxo, and alkoxy. In another preferred embodiment, R³⁷ is selected from the group consisting of 2,6-dialkoxyaryl, 2,6-dialkoxyheteroaryl, 2-alkyl-6-alkoxyaryl, 2-alkyl-6-alkoxyheteroaryl, 2-oxo-6-alkoxyheteroaryl, 2-oxo-6-alkylheteroaryl, and optionally substituted imidazolidin-2,4-dion-3-yl.

Preferably in the compounds of formula II above, Ar³¹ is selected from the group consisting of 4-methylphenyl, 4-chlorophenyl, 1-naphthyl, 2-naphthyl, 4-methoxyphenyl, phenyl, 2,4,6-trimethylphenyl, 2-(methoxycarbonyl)phenyl, 2-carboxyphenyl, 3,5-dichlorophenyl, 4-trifluoromethylphenyl, 3,4-dichlorophenyl, 3,4-dimethoxyphenyl, 4-(CH₃C(O)NH-)phenyl, 4-trifluoromethoxyphenyl, 4-cyanophenyl, 3,5-di-(trifluoromethyl)phenyl, 4-t-butylphenyl, 4-t-butoxyphenyl, 4-nitrophenyl, 2-thienyl, 1-N-methyl-3-methyl-5-chloropyrazol-4-yl, 1-N-methylimidazol-4-yl, 4-bromophenyl, 4-amidinophenyl, 4-methylamidinophenyl, 4-[CH₃SC(═NH)]phenyl, 5-chloro-2-thienyl, 2,5-dichloro-4-thienyl, 1-N-methyl-4-pyrazolyl, 2-thiazolyl, 5-methyl-1,3,4-thiadiazol-2-yl, 4-[H₂NC(S)]phenyl, 4-aminophenyl, 4-fluorophenyl, 2-fluorophenyl, 3-fluorophenyl, 3,5-difluorophenyl, pyridin-3-yl, pyrimidin-2-yl, 4-(3′-dimethylamino-n-propoxy)-phenyl, and 1-methylpyrazol-4-yl.

When describing the compounds of formulae I and II, compositions comprising compound of formulae I and II, and methods of this invention for compounds of formulae I and II, the following terms have the following meanings, unless otherwise indicated.

Definitions

As used herein, “acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenoxy” refers to the group “alkenyl-O—”.

“Substituted alkenoxy” refers to the group “substituted alkenyl-O—”.

“Alkenyl” refers to alkenyl group preferably having from 2 to 10 carbon atoms and more preferably 2 to 6 carbon atoms and having at least 1 and preferably from 1-2 sites of alkenyl unsaturation.

“Substituted alkenyl” refers to alkenyl groups having from 1 to 5 substituents independently selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, cycloalkyloxy, substituted cycloalkyloxy, heteroaryloxy, substituted heteroaryloxy, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Preferably, the substituents are independently selected from the group consisting of alkoxy, substituted alkoxy, acyl, acylamino, acyloxy, amino, substituted amino, aminoacyl, aminocarbonylamino, aminocarbonyloxy, aryl, substituted aryl, aryloxy, substituted aryloxy, carboxyl, carboxyl esters, cyano, cycloalkyl, substituted cycloalkyl, cycloalkyloxy, substituted cycloalkyloxy, halogen, heteroaryl, substituted heteroaryl, heteroaryloxy, substituted heteroaryloxy, heterocyclic, substituted heterocyclic, hydroxyl, nitro, and oxycarbonylamino.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Alkyl” refers to alkyl groups preferably having from 1 to 10 carbon atoms and more preferably 1 to 6 carbon atoms. This term is exemplified by groups such as methyl, t-butyl, n-heptyl, octyl and the like.

“Substituted alkyl” refers to an alkyl group, of from 1 to 10 carbon atoms, having from 1 to 5 substituents independently selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, cycloalkyloxy, substituted cycloalkyloxy, heteroaryloxy, substituted heteroaryloxy, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Preferably, the substituents are independently selected from the group consisting of alkoxy, substituted alkoxy, acyl, acylamino, acyloxy, amino, substituted amino, aminoacyl, aminocarbonylamino, aminocarbonyloxy, aryl, substituted aryl, aryloxy, substituted aryloxy, carboxyl, carboxyl esters, cyano, cycloalkyl, substituted cycloalkyl, cycloalkyloxy, substituted cycloalkyloxy, halogen, heteroaryl, substituted heteroaryl, heteroaryloxy, substituted heteroaryloxy, heterocyclic, substituted heterocyclic, hydroxyl, nitro, and oxycarbonylamino.

“Alkylene” refers to linear and branched divalent alkyl groups having from 1 to 10 carbon atoms and more preferably 1 to 6 carbon atoms. This term is exemplified by groups such as methylene (—CH₂—), 1,6-heptylene, 1,8-octylene, ethylene (—CH₂CH₂—), the propylene isomers (e.g., —CH₂CH₂CH₂— and —CH(CH₃)CH₂—) and the like.

“Substituted alkylene” refers to alkylene groups having from 1 to 5 substituents independently selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynyl” refers to alkynyl group preferably having from 2 to 10 carbon atoms and more preferably 3 to 6 carbon atoms and having at least 1 and preferably from 1-2 sites of alkynyl unsaturation.

“Substituted alkynyl” refers to alkynyl groups having from 1 to 5 substituents independently selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic, substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Amidino” refers to the group H₂NC(═NH)— and the term “alkylamidino” refers to compounds having 1 to 3 alkyl groups (e.g., alkylHNC(═NH)—).

“Amino” refers to the group —NH₂.

“Substituted amino” refers to the group —NRR, where each R group is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic, and substituted heterocyclic, provided that both R groups are not hydrogen; or where the R groups can be joined together with the nitrogen atom to form a heterocyclic or substituted heterocyclic ring.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the groups —NRC(O)NRR, —NRC(O)NR-alkyl, —NRC(O)NR-substituted alkyl, —NRC(O)NR-alkenyl, —NRC(O)NR-substituted alkenyl, —NRC(O)NR-alkynyl, —NRC(O)NR-substituted alkynyl, —NRC(O)NR-aryl, —NRC(O)NR-substituted aryl, —NRC(O)NR-cycloalkyl, —NRC(O)NR-substituted cycloalkyl, —NRC(O)NR-heteroaryl, and —NRC(O)NR-substituted heteroaryl, —NRC(O)NR-heterocyclic, and —NRC(O)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the groups —NRC(O)O-alkyl, —NRC(O)O-substituted alkyl, —NRC(O)O-alkenyl, —NRC(O)O-substituted alkenyl, —NRC(O)O-alkynyl, —NRC(O)O-substituted alkynyl, —NRC(O)O-cycloalkyl, —NRC(O)O-substituted cycloalkyl, —NRC(O)O-aryl, —NRC(O)O-substituted aryl, —NRC(O)O-heteroaryl, —NRC(O)O-substituted heteroaryl, —NRC(O)O-heterocyclic, and —NRC(O)O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the groups —NRC(S)NRR, —NRC(S)NR-alkyl, —NRC(S)NR-substituted alkyl, —NRC(S)NR-alkenyl, —NRC(S)NR-substituted alkenyl, —NRC(S)NR-alkynyl, —NRC(S)NR-substituted alkynyl, —NRC(S)NR-aryl, —NRC(S)NR-substituted aryl, —NRC(S)NR-cycloalkyl, —NRC(S)NR-substituted cycloalkyl, —NRC(S)NR-heteroaryl, and —NRC(S)NR-substituted heteroaryl, —NRC(S)NR-heterocyclic, and —NRC(S)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3 (4H)-one-7yl, and the like) provided that the point of attachment is through an aromatic ring atom. Preferred aryls include phenyl, naphthyl and 5,6,7,8-tetrahydronaphth-2-yl.

“Substituted aryl” refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

Preferred substituents are selected from the group consisting of hydroxy, acyl, acylamino, acyloxy, alkyl, substituted alkyl, alkoxy, substituted alkoxy, alkenyl, substituted alkenyl, amino, substituted amino, aminoacyl, aminocarbonyloxy, aminocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters, cyano, cycloalkyl, substituted cycloalkyl, halo, nitro, heteroaryl, substituted heteroaryl, heterocyclic, substituted heterocyclic, and oxycarbonylamino.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Aralkoxy” refers to aryl-alkylene-O— groups.

“Substituted aralkoxy” refers to substituted aryl-alkylene-O— groups.

“Carboxyl” refers to the group —COOH and pharmaceutically acceptable salts thereof.

“Carboxyl esters” refers —C(O)O-alkyl, —C(O)O-substituted alkyl, —C(O)O-alkenyl, —C(O)O-substituted alkenyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic.

“Cycloalkenyl” refers to cyclic alkenyl groups of form 3 to 8 carbon atoms having a single cyclic ring.

“Cycloalkoxy” refers to —O-cycloalkyl groups.

“Substituted cycloalkoxy” refers to —O-substituted cycloalkyl groups.

“Cycloalkyl” refers to cyclic alkyl groups of from 3 to 12 carbon atoms having a single or multiple condensed rings including, by way of example, adamantyl, cyclopropyl, cyclobutyl, cyclopentyl, cyclohexyl, cyclooctyl and the like. Preferably “cycloalkyl” refers to cyclic alkyl groups of from 3 to 8 carbon atoms having a single cyclic ring.

“Substituted cycloalkyl” and “substituted cycloalkenyl” refers to an cycloalkyl or cycloalkenyl group, preferably of from 3 to 8 carbon atoms, having from 1 to 5 substituents independently selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Preferred substituents are selected from the group consisting of oxo (═O), thioxo (═S), alkoxy, substituted alkoxy, acyl, acylamino, acyloxy, amino, substituted amino, aminoacyl, aminocarbonylamino, aminocarbonyloxy, aryl, substituted aryl, aryloxy, substituted aryloxy, carboxyl, carboxyl esters, cyano, cycloalkyl, substituted cycloalkyl, cycloalkyloxy, substituted cycloalkyloxy, halogen, heteroaryl, substituted heteroaryl, heteroaryloxy, substituted heteroaryloxy, heterocyclic, substituted heterocyclic, hydroxyl, nitro, and oxycarbonylamino.

“Guanidino” refers to the groups —NRC(═NR)NRR, —NRC(═NR)NR-alkyl, —NRC(═NR)NR-substituted alkyl, —NRC(═NR)NR-alkenyl, —NRC(═NR)NR-substituted alkenyl, —NRC(═NR)NR-alkynyl, —NRC(═NR)NR-substituted alkynyl, —NRC(═NR)NR-aryl, —NRC(═NR)NR-substituted aryl, —NRC(═NR)NR-cycloalkyl, —NRC(═NR)NR-heteroaryl, —NRC(═NR)NR-substituted heteroaryl, —NRC(═NR)NR-heterocyclic, and —NRC(═NR)NR-substituted heterocyclic where each R is independently hydrogen and alkyl as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Guanidinosulfone” refers to the groups —NRC(═NR)NRSO₂-alkyl, —NRC(═NR)NRSO₂-substituted alkyl, —NRC(═NR)NRSO₂-alkenyl, —NRC(═NR)NRSO₂-substituted alkenyl, —NRC(═NR)NRSO₂-alkynyl, —NRC(═NR)NRSO₂-substituted alkynyl, —NRC(═NR)NRSO₂-aryl, —NRC(═NR)NRSO₂-substituted aryl, —NRC(═NR)NRSO₂-cycloalkyl, —NRC(═NR)NRSO₂-substituted cycloalkyl, —NRC(═NR)NRSO₂-heteroaryl, and —NRC(═NR)NRSO₂-substituted heteroaryl, —NRC(═NR)NRSO₂-heterocyclic, and —NRC(═NR)NRSO₂-substituted heterocyclic where each R is independently hydrogen and alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Halo” or “halogen” refers to fluoro, chloro, bromo and iodo and preferably is fluoro, chloro or bromo.

“Heteroaryl” refers to an aromatic carbocyclic group of from 2 to 10 carbon atoms and 1 to 4 heteroatoms selected from the group consisting of oxygen, nitrogen and sulfur within the ring or oxides thereof. Such heteroaryl groups can have a single ring (e.g., pyridyl or furyl) or multiple condensed rings (e.g., indolizinyl or benzothienyl) wherein one or more of the condensed rings may or may not be aromatic provided that the point of attachment is through an aromatic ring atom. Additionally, the heteroatoms of the heteroaryl group may be oxidized, i.e., to form pyridine N-oxides or 1,1-dioxo-1,2,5-thiadiazoles and the like. Additionally, the carbon atoms of the ring may be substituted with an oxo (═O). Preferred heteroaryls include pyridyl, pyrrolyl, indolyl, furyl, pyridazinyl, pyrimidinyl, pyrazinyl, 1-oxo-1,2,5-thiadiazolyl and 1,1-dioxo-1,2,5-thiadiazolyl.

“Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

Preferably the substituents are selected from the group consisting of those defined above as preferred for substituted aryl.

“Heteroaryloxy” refers to the group —O-heteroaryl and “substituted heteroaryloxy” refers to the group —O-substituted heteroaryl.

“Heteroaralkoxy” refers to the group heteroaryl-alkylene-O—.

“Substituted heteroaralkoxy” refers to the group substituted heteroaryl-alkylene-O—.

“Heterocycle” or “heterocyclic” refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclic” refers to heterocycle groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, —C(O)O-aryl, —C(O)O-substituted aryl, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Preferably, the substituents are selected from the group consisting of the preferred substitutents defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholino, morpholinyl, thiomorpholino, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“N,N-Dimethylcarbamyloxy” refers to the group —OC(O)N(CH₃)₂.

“Oxo” refers to (═O).

“Oxyalkylene” refers to —OCH₂CHR^{d}— where R^{d} is alkyl.

“Oxycarbonylamino” refers to the groups —OC(O)NH₂, —OC(O)NRR, —OC(O)NR-alkyl, —OC(O)NR-substituted alkyl, —OC(O)NR-alkenyl, —OC(O)NR-substituted alkenyl, —OC(O)NR-alkynyl, —OC(O)NR-substituted alkynyl, —OC(O)NR-cycloalkyl, —OC(O)NR-substituted cycloalkyl, —OC(O)NR-aryl, —OC(O)NR-substituted aryl, —OC(O)NR-heteroaryl, —OC(O)NR-substituted heteroaryl, —OC(O)NR-heterocyclic, and —OC(O)NR-substituted heterocyclic where R is hydrogen, alkyl or where each R is joined to form, together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NH₂, —OC(S)NRR, —OC(S)NR-alkyl, —OC(S)NR-substituted alkyl, —OC(S)NR-alkenyl, —OC(S)NR-substituted alkenyl, —OC(S)NR-alkynyl, —OC(S)NR-substituted alkynyl, —OC(S)NR-cycloalkyl, —OC(S)NR-substituted cycloalkyl, —OC(S)NR-aryl, —OC(S)NR-substituted aryl, —OC(S)NR-heteroaryl, —OC(S)NR-substituted heteroaryl, —OC(S)NR-heterocyclic, and —OC(S)NR-substituted heterocyclic where R is hydrogen, alkyl or where each R is joined to form together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioalkyl” refers to the groups —S-alkyl.

“Substituted thioalkyl” refers to the group —S-substituted alkyl.

“Thioamidino” refers to the group RSC(═NH)— where R is hydrogen or alkyl.

“Thioaryl” refers to the group —S-aryl and “substituted thioaryl” refers to the group —S-substituted aryl.

“Thiocarbonylamino” refers to the group —C(S)NRR where each R is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form, together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocycloalkyl” refers to the groups —S-cycloalkyl.

“Substituted thiocycloalkyl” refers to the group —S-substituted cycloalkyl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

“Thiol” refers to the group —SH.

“Optionally subsituted” means that the recited group may be unsubstituted or the recited group may be substituted.

The compounds of formulae I and II of this invention can be prepared from readily available starting materials as described in U.S. Pat. Nos. 6,489,300 and 6,583,139 and U.S. Patent Publication 2004/0006093 using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Compounds of Formulae IIIa, IIIb, IVa IVb, IVc, IVd, Va, Vb, Vc, Vd, VIa, VIb, VIc, and VId

In one aspect, the compounds that can be utilized as steroid sparing agents for treatment of a subject, with a disease selected from the group consisting of rheumatoid arthritis, asthma, graft versus host disease, host versus graft disease, and spondyloarthropathies, are compounds of formulae IIIa, IIIb, IVa, IVb, IVc, IVd, Va, Vb, Vc, Vd, VIa, VIb, VIc, and VId. Preferably, the compounds of formulae IIIa, IIIb, IVa, IVb, IVc, IVd, Va, Vb, Vc, Vd, VIa, VIb, VIc, and VId can be utilized as steriod sparing agents for treatment of a subject with a disease selected from the group consisting of spondyloarthropathies and rheumatoid arthritis.

In one embodiment, the compounds that can be utilized as steroid sparing agents are compounds defined by formula IIIa and/or IIIb below. 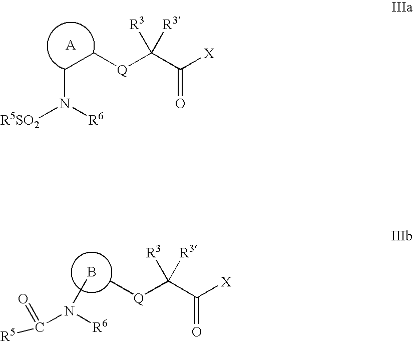
where R³ and R^{3′} are independently selected from the group consisting of hydrogen, isopropyl, —CH₂Z where Z is selected from the group consisting of hydrogen, hydroxyl, acylamino, alkyl, alkoxy, aryloxy, aryl, aryloxyaryl, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted alkyl, substituted alkoxy, substituted aryl, substituted aryloxy, substituted aryloxyaryl, substituted cycloalkyl, heteroaryl, substituted heteroaryl, heterocyclic and substituted heterocyclic, and
where R³ and R^{3′} are joined to form a substituent selected from the group consisting of ═CHZ where Z is defined above provided that Z is not hydroxyl or thiol, cycloalkyl, substituted cycloalkyl, cycloalkenyl, substituted cycloalkenyl, heterocyclic and substituted heterocyclic;
Q is selected from the group consisting of —O—, —S—, —S(O)—, —S(O)₂—, and —NR⁴—;
X is selected from the group consisting of hydroxyl, alkoxy, substituted alkoxy, alkenoxy, substituted alkenoxy, cycloalkoxy, substituted cycloalkoxy, cycloalkenoxy, substituted cycloalkenoxy, aryloxy, substituted aryloxy, heteroaryloxy, substituted heteroaryloxy, heterocyclyloxy, substituted heterocyclyloxy and —NR″R″ where each R″ is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic and substituted heterocyclic;
ring A and ring B independently form a heteroaryl or substituted heteroaryl group having two nitrogen atoms in the heteroaryl ring;
R⁴ is selected from the group consisting of hydrogen, alkyl, substituted alkyl, alkenyl, substituted alkenyl, cycloalkyl, substituted cycloalkyl, cycloalkenyl, substituted cycloalkenyl, aryl, substituted aryl, heteroaryl, substituted heteroaryl, heterocyclic and substituted heterocylic;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl;
or optionally, one of, R⁴ and ring A, R⁴ and R⁵, R⁴ and R⁶, or R⁵ and R⁶, together with the atoms to which they are bound, can be joined to form a heterocyclic or substituted heterocyclic ring;
provided that ring B does not form a 6-amino or substituted amino pyrimidin-4-yl group;
and enantiomers, diastereomers and pharmaceutically acceptable salts thereof.

Preferably, ring A forms a pyridazine, pyrimidine or pyrazine ring; more preferably, a pyrimidine or pyrazine ring; wherein the pyridazine, pyrimidine or pyrazine ring is optionally substituted with 1 to 3 substituents selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen.

Preferably, ring B forms a pyridazine, pyrimidine, pyrazine; more preferably, a pyrimidine, pyrazine; wherein the pyridazine, pyrimidine or pyrazine ring is optionally substituted with 1 to 3 substituents selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen.

Preferably, R³ is —(CH₂)ₓ—Ar—R⁹, where Ar is aryl, substituted aryl, heteroaryl and substituted heteroaryl; R⁹ is selected from the group consisting of acyl, acylamino, acyloxy, aminoacyl, aminocarbonylamino, aminothiocarbonylamino, aminocarbonyloxy, oxythiocarbonylamino, thioamidino, thiocarbonylamino, aminosulfonylamino, aminosulfonyloxy, aminosulfonyl, oxysulfonylamino and oxysulfonyl; and x is an integer from 0 to 4. R^{3′} is preferably alkyl or hydrogen; more preferably, R^{3′} is hydrogen.

More preferably, R³ is a group of the formula: 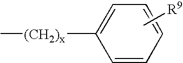
wherein R⁹ and x are as defined herein. Preferably, R⁹ is in the para position of the phenyl ring; and x is an integer of from 1 to 4, more preferably, x is 1.

In a preferred embodiment, R⁹ is selected from —O-Z-NR¹¹R^{11′} and —O-Z-R¹² wherein R¹¹ and R^{11′} are independently selected from the group consisting of hydrogen, alkyl, substituted alkyl, cycloalkyl, substituted cycloalkyl, cycloalkenyl, substituted cycloalkenyl, heterocyclic, substituted heterocyclic, and where R¹¹ and R¹¹ are joined to form a heterocycle or a substituted heterocycle, R¹² is selected from the group consisting of heterocycle and substituted heterocycle, and Z is selected from the group consisting of —C(O)— and —SO₂—. More preferably, R⁹ is —OC(O)NR″R¹¹, wherein R¹¹ and R^{11′} are as defined herein.
Z is preferably —C(O)—. Preferably, Q is —NR⁴—;

In another embodiment, the compounds that can be utilized as steroid sparing agents are compounds defined by formula IVa, IVb, IVc, or IVd: 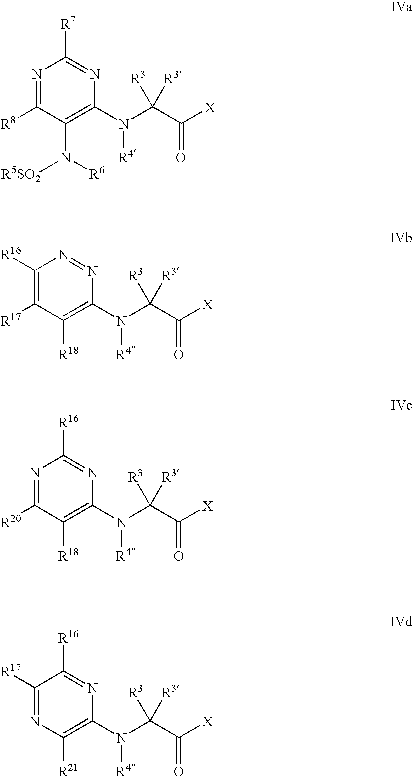
wherein R³, R^{3′} and X are as defined herein;
R^{4′} is selected from the group consisting of hydrogen and alkyl or, optionally, one of, R^{4′} and R⁵, R^{4′} and R⁶, R⁵ and R⁶, R⁵ and R⁸, or R⁶ and R⁸, together with the atoms to which they are bound, are joined to form a heterocyclic, a substituted heterocyclic, a heteroaryl or substituted heteroaryl group optionally containing from 1 to 3 additional hetero ring atoms selected from the group consisting of oxygen, nitrogen and sulfur;
R^{4″} is selected from the group consisting of hydrogen and alkyl;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl;
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enantiomers, diastereomers and pharmaceutically acceptable salts thereof.

In another embodiment, the compounds that can be utilized as steroid sparing agents are compounds defined by formula Va, Vb, Vc, or Vd: 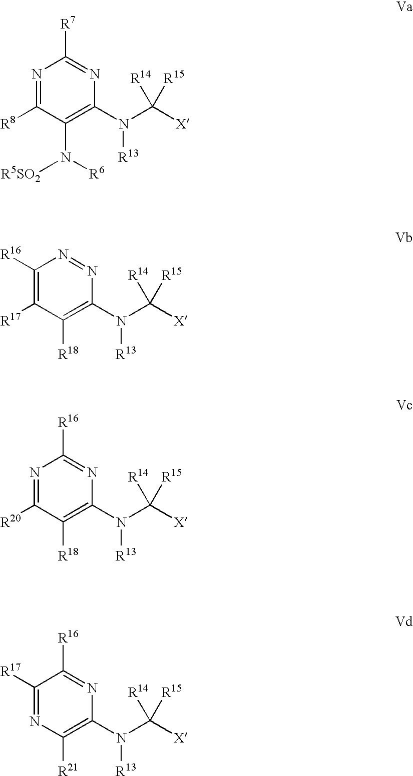
wherein:
R¹³ is selected from the group consisting of hydrogen, C₁₋₁₀ alkyl, Cy, and Cy-C₁₋₁₀ alkyl, wherein alkyl is optionally substituted with one to four substituents independently selected from R^{a}; and Cy is optionally substituted with one to four substituents independently selected from R^{b};
R¹⁴ is selected from the group consisting of hydrogen, C₁₋₁₀ alky, C₂₋₁₀ alkenyl, C₂₋₁₀ alkynyl, Cy, Cy-C₁₋₁₀ alkyl, Cy-C₂₋₁₀ alkenyl and Cy-C₂₋₁₀ alkynyl, wherein alkyl, alkenyl, and alkynyl are optionally substituted with one to four substituents selected from phenyl and R^{x}, and Cy is optionally substituted with one to four substituents independently selected from R^{y};
or R¹³, R¹⁴ and the atoms to which they are attached together form a mono- or bicyclic ring containing 0-2 additional heteratoms selected from N, O and S;
R¹⁵ is selected from the group consisting of C₁₋₁₀ alkyl, C₂₋₁₀ alkenyl, C₂₋₁₀ alkynyl, aryl, aryl-C₁₋₁₀ alkyl, heteroaryl, heteroaryl-C₁₋₁₀ alkyl, wherein alkyl, alkenyl and alkynyl are optionally substituted with one to four substituents selected from R^{x}, and aryl and heteroaryl are optionally substituted with one to four substituents independently selected from R^{y};
or R¹⁴, R¹⁵ and the carbon to which they are attached form a 3-7 membered mono- or bicyclic ring containing 0-2 heteroatoms selected from N, O and S;
R^{a} is selected from the group consisting of Cy and a group selected from R^{x}, wherein Cy is optionally substituted with one to four substituents independently selected from R^{c};
R^{b} is selected from the group consisting of R^{a}, C₁₋₁₀ alkyl, C₂₋₁₀ alkenyl, C₂₋₁₀ alkynyl, aryl C₁₋₁₀alkyl, heteroaryl C₁₋₁₀ alkyl, wherein alkyl, alkenyl, alkynyl, aryl, heteroaryl are optionally substituted with a group independently selected from R^{c};
R^{c} is selected from the group consisting of halogen, NO₂, C(O)OR^{f}, C₁₋₄ alkyl, C₁₋₄ alkoxy, aryl, aryl C₁₋₄ alkyl, aryloxy, heteroaryl, NR^{f}R^{g}, R^{f}C(O)R^{g}, NR^{f}C(O)NR^{f}R^{g}, and CN;
R^{d} and R^{e} are independently selected from hydrogen, C₁₋₁₀ alkyl, C₂₋₁₀ alkenyl, C₂₋₁₀ alkynyl, Cy and Cy C₁₋₁₀alkyl, wherein alkyl, alkenyl, alkynyl and Cy are optionally substituted with one to four substituents independently selected from R^{c};
or R and R^{e} together with the atoms to which they are attached form a heterocyclic ring of 5 to 7 members containing 0-2 additional heteroatoms independently selected from oxygen, sulfur and nitrogen;
R^{f} and R^{g} are independently selected from hydrogen, C₁₋₁₀ alkyl, Cy and Cy-C₁₋₁₀ alkyl wherein Cy is optionally substituted with C₁₋₁₀ alkyl; or R^{f} and R^{g} together with the carbon to which they are attached form a ring of 5 to 7 members containing 0-2 heteroatoms independently selected from oxygen, sulfur and nitrogen;
R^{h} is selected from the group consisting of hydrogen, C₁₋₁₀alkyl, C₂₋₁₀ alkenyl, C₂₋₁₀ alkynyl, cyano, aryl, aryl C₁₋₁₀ alkyl, heteroaryl, heteroaryl C₁₋₁₀ alkyl, and —SO₂Rⁱ; wherein alkyl, alkenyl, and alkynl are optionally substituted with one to four substitutents independently selected from R^{a}; and aryl and heteroaryl are each optionally substituted with one to four substituents independently selected from R^{b};
Rⁱ is selected from the group consisting of C₁₋₁₀ alkyl, C₂₋₁₀ alkenyl, C₂₋₁₀ alkynyl, and aryl; wherein alkyl, alkenyl, alkynyl and aryl are each optionally substituted with one to four substituents independently selected from R^{c};
R^{x} is selected from the group consisting of —OR^{d}, —NO₂, halogen, —S(O)ₘR^{d}, —SR^{d}, —S(O)₂OR^{d}, —S(O)ₘNR^{d}Rₑ, —NR^{d}R^{e}, —O(CR^{f}R^{g})ₙNR^{d}R^{e}, —C(O)R^{d}, —CO₂R^{d}, —CO₂(CR^{f}R^{g})ₙCONR^{d}R, —OC(O)R^{d}, —CN, —C(O)NR^{d}R^{e}, —NR^{d}C(O)R^{c}, —OC(O)NR^{d}R, —NR^{d}C(O)OR^{c}, —NR DC(O)NR^{d}R^{e}, —CR^{d}(N—OR^{e}), CF₃, oxo, NR^{d}C(O)NR^{d}SO₂R¹, NR^{d}S(O)ₘR^{e}, —OS(O)₂OR^{d}, and —OP(O)(OR^{d})₂;
R^{y} is selected from the group consisting of R^{x}, C₁₋₁₀ alkyl, C₂₋₁₀ alkenyl, C₂₋₁₀ alkynyl, aryl C₁₋₁₀alkyl, heteroaryl C₁₋₁₀ alkyl, cycloalkyl, heterocyclyl; wherein alkyl, alkenyl, alkynyl and aryl are each optionally substituted with one to four substitutents independently selected from R^{x};
Cy is cycloalkyl, heterocyclyl, aryl, or heteroaryl;
m is an integer from 1 to 2;
n is an integer from 1 to 10;
X′ is selected from the group consisting of —C(O)OR^{d}, —P(O)(OR^{d})(OR^{e}), —P(O)(R^{d})(OR^{e}), —S(O)ₘOR^{d}, C(O)NR^{d}R^{h}, and -5-tetrazolyl;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl; and
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enatiomers, diastereomers and pharmaceutically acceptable salts thereof.

Preferably, X′ is —C(O)OR^{d}.

In another embodiment, the compounds that can be utilized as steroid sparing agents are compounds defined by formula VIa, VIb, VIc, or VId: 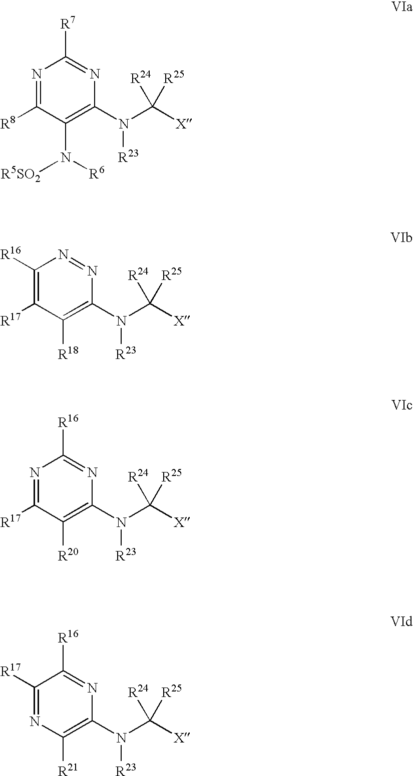
wherein:
R²³ is selected from the group consisting of hydrogen, C₁₋₁₀ alkyl optionally substituted with one to four substituents independently selected from R^{a} and Cy optionally substituted with one to four substituents independently selected from R^{b′};
R²⁴ is selected from the group consisting of Ar¹—Ar²—C₁₋₁₀ alkyl, Ar¹—Ar²—C₂₋₁₀ alkenyl, Ar¹—Ar²—C₂₋₁₀ alkynyl, wherein Ar and Ar² are independently aryl or heteroaryl each of which is optionally substituted with one to four substituents independently selected from R^{b′}; alkyl, alkenyl and alkynyl are optionally substituted with one to four substituents independently selected from R^{a};
R²⁵ is selected from the group consisting of hydrogen, C₁₋₁₀alkyl, C₂₋₁₀ alkenyl, C₂₋₁₀ alkynyl, aryl, aryl C₁₋₁₀alkyl, heteroaryl, and heteroaryl C₁₋₁₀alkyl, wherein alkyl, alkenyl and alkynyl are optionally substituted with one to four substituents selected from R^{a′}, and aryl and heteroaryl are optionally substituted with one to four substituents independently selected from R^{b′};
R^{a′} is selected from the group consisting of Cy, —OR^{d′}, —NO₂, halogen —S(O)ₘR^{d′}, —SR^{d′}, —S(O)₂OR^{d′}, S(O)ₘNR^{d′}R^{e′}, —NR^{d′}R^{e′}, —O(CR^{f′}R^{g′})ₙNR^{d′}R^{e′}, —C(O)R^{d}, —CO₂R^{d′}, —CO₂(CR^{f′}R^{g′})ₙCONR^{d′}R^{e′}, —OC(O)R^{d′}, —CN, —C(O)NR^{d′}R^{e′}, —NR^{d′}C(O)R^{e′}, —OC(O)NR^{d′}R^{e′}, —NR^{d′}C(O)OR^{e′}, —NR^{d′}C(O)NR^{d′}R^{e′}, —CR^{d′}(N—OR^{e′}), CF₃, and —OCF₃;
wherein Cy is optionally substituted with one to four substituents independently selected from R^{c′};
R^{b′} is selected from the group consisting of R^{a′}, C₁₋₁₀ alkyl, C₂₋₁₀ alkenyl, C₂₋₁₀ alkynyl, aryl C₁₋₁₀ alkyl, heteroaryl C₁₋₁₀alkyl,
wherein alkyl, alkenyl, aryl, heteroaryl are optionally substituted with a group independently selected from R^{c′};
R^{c′} is selected from the group consisting of halogen, amino, carboxy, C₁₋₄ alkyl, C₁₋₄ alkoxy, aryl, aryl C₁₋₄-alkyl, hydroxy, CF₃, and aryloxy;
R^{d′} and R^{e′} are independently selected from hydrogen, C₁₋₁₀ alkyl, C₂₋₁₀ alkenyl, C₂₋₁₀ alkynyl, Cy and Cy C₁₋₁₀alkyl, wherein alkyl, alkenyl, alkynyl and Cy are optionally substituted with one to four substituents independently selected from R^{e′}; or R^{d′} and R^{e′} together with the atoms to which they are attached form a heterocyclic ring of 5 to 7 members containing 0-2 additional heteroatoms independently selected from oxygen, sulfur and nitrogen;
R^{f′} and R^{g′} are independently selected from hydrogen, C₁₋₁₀ alkyl, Cy and Cy-C₁₋₁₀ alkyl; or R^{f′} and R^{g′} together with the carbon to which they are attached form a ring of 5 to 7 members containing 0-2 heteroatoms independently selected from oxygen, sulfur and nitrogen;
R^{h′} is selected from the group consisting of hydrogen, C₁₋₁₀ alkyl, C₂₋₁₀ alkenyl, C₂₋₁₀ alkynyl, cyano, aryl, aryl C₁₋₁₀ alkyl, heteroaryl, heteroaryl C₁₋₁₀ alkyl, or —SO₂R^{i′};
wherein alkyl, alkenyl, and alkynyl are optionally substituted with one to four substitutents independently selected from R^{a}; and aryl and heteroaryl are each optionally substituted with one to four substituents independently selected from R^{b′};
R^{i′} is selected from the group consisting of C₁₋₁₀ alkyl, C₂₋₁₀ alkenyl, C₂₋₁₀ alkynyl, and aryl;
wherein alkyl, alkenyl, alkynyl and aryl are each optionally substituted with one to four substituents independently selected from R^{c′};
Cy is cycloalkyl, heterocyclyl, aryl, or heteroaryl;
X″ is selected from the group consisting of —C(O)OR^{d′}, —P(O)(OR^{d′})(OR^{e′}), —P(O)(R^{d′})(OR^{e′}), —S(O)ₘOR^{d′}, —C(O)NR^{d′}R^{h′}, and -5-tetrazolyl;
m is an integer from 1 to 2;
n is an integer from 1 to 10.
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl; and
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enantiomers, diastereomers and pharmaceutically acceptable salts thereof.

Preferably, X″ is —C(O)OR^{d}.

Preferably, R²⁴ is —CH₂—Ar²—Ar¹ and R²⁵ is hydrogen.

In the above compounds IIIa, IIIb, IVa, IVb, IVc, and IVd, when X is other than —OH or pharmaceutical salts thereof, X is preferably a substituent which will convert (e.g., hydrolyze, metabolize, etc.) in vivo to a compound where X is —OH or a salt thereof. Accordingly, suitable X groups are any art recognized pharmaceutically acceptable groups which will hydrolyze or otherwise convert in vivo to a hydroxyl group or a salt thereof including, by way of example, esters (X is alkoxy, substituted alkoxy, cycloalkoxy, substituted cycloalkoxy, alkenoxy, substituted alkenoxy, cycloalkenoxy, substituted cycloalkenoxy, aryloxy, substituted aryloxy, heteroaryloxy, substituted heteroaryloxy, heterocyclooxy, substituted heterocyclooxy, and the like).

Unless otherwise defined, R³ and R¹⁵ in the above compounds are preferably selected from all possible isomers arising by substitution with the following groups:
4-methylbenzyl,
4-hydroxybenzyl,
4-methoxybenzyl,
4-t-butoxybenzyl,
4-benzyloxybenzyl,
4-[φ-CH(CH₃)O-]benzyl,
4-[φ-CH(COOH)O-]benzyl,
4-[BocNHCH₂C(O)NH-]benzyl,
4-chlorobenzyl,
4-[NH₂CH₂C(O)NH-]benzyl,
4-carboxybenzyl,
4-[CbzNHCH₂CH₂NH-]benzyl,
3-hydroxy-4-(φ-OC(O)NH-)benzyl,
4-[HOOCCH₂CH₂C(O)NH-]benzyl,
benzyl,
4-[2′-carboxylphenoxy-]benzyl,
4-[φ-C(O)NH-]benzyl,
3-carboxybenzyl,
4-iodobenzyl,
4-hydroxy-3,5-diiodobenzyl,
4-hydroxy-3-iodobenzyl,
4-[2′-carboxyphenyl-]benzyl,
φ-CH₂CH₂—,
4-nitrobenzyl,
2-carboxybenzyl,
4-[dibenzylamino]-benzyl,
4-[(1′-cyclopropylpiperidin-4′-yl)C(O)NH-]benzyl,
4-[-NHC(O)CH₂NHBoc]benzyl,
4-carboxybenzyl,
4-hydroxy-3-nitrobenzyl,
4-[-NHC(O)CH(CH₃)NHBoc]benzyl,
4-[-NHC(O)CH(CH₂φ)NHBoc]benzyl,
isobutyl,
methyl,
4-[CH₃C(O)NH-]benzyl,
—CH₂-(3-indolyl),
35 n-butyl,
t-butyl-OC(O)CH₂—,
t-butyl-OC(O)CH₂CH₂—,
H₂NC(O)CH₂—,
H₂NC(O)CH₂CH₂—,
40 BocNH—(CH₂)₄—,
t-butyl-OC(O)—(CH₂)₂—,
HOOCCH₂—,
HOOC(CH₂)₂—,
H₂N(CH₂)₄—,
45 isopropyl,
(1-naphthyl)-CH₂—,
(2-naphthyl)-CH₂—,
(2-thiophenyl)-CH₂—,
(φ-CH₂—OC(O)NH—(CH₂)₄—,
cyclohexyl-CH₂—,
benzyloxy-CH₂—,
HOCH₂—,
5-(3-N-benzyl)imidazolyl-CH₂—,
2-pyridyl-CH₂—,
3-pyridyl-CH₂—,
4-pyridyl-CH₂—,
5-(3-N-methyl)imidazolyl-CH₂—,
N-benzylpiperid-4-yl-CH₂—,
N-Boc-piperidin-4-yl-CH₂—,
N-(phenyl-carbonyl)piperidin-4-yl-CH₂—,
H₃CSCH₂CH₂—,
1-N-benzylimidazol-4-yl-CH₂—,
iso-propyl-C(O)NH—(CH₂)₄—,
iso-butyl-C(O)NH—(CH₂)₄—,
phenyl-C(O)NH—(CH₂)₄—,
benzyl-C(O)NH—(CH₂)₄—,
allyl-C(O)NH—(CH₂)₄—,
4-(3-N-methylimidazolyl)-CH₂—,
4-imidazolyl,
4-[(CH₃)₂NCH₂CH₂CH₂—O-]benzyl,
4-[(benzyl)₂N-]-benzyl,
4-aminobenzyl,
allyloxy-C(O)NH(CH₂)₄—,
allyloxy-C(O)NH(CH₂)₃—,
allyloxy-C(O)NH(CH₂)₂—,
NH₂C(O)CH₂—,
φ-CH═,
2-pyridyl-C(O) NH—(CH₂)₄—,
4-methylpyrid-3-yl-C(O)NH—(CH₂)₄—,
3-methylthien-2-yl-C(O)NH—(CH₂)₄—,
2-pyrrolyl-C(O)NH—(CH₂)₄—,
2-furanyl-C(O)NH—(CH₂)₄—,
4-methylphenyl-SO₂—N(CH₃)CH₂C(O)NH(CH₂)₄—,
4-[cyclopentylacetylenyl]-benzyl,
4-[-NHC(O)-(N-Boc)-pyrrolidin-2-yl)]-benzyl-,
1-N-methylimidazol-4-yl-CH₂—,
1-N-methylimidazol-5-yl-CH₂—,
imidazol-5-yl-CH₂—,
6-methylpyrid-3-yl-C(O)NH—(CH₂)₄—,
4-[2′-carboxymethylphenyl]-benzyl,
4-[-NHC(O)NHCH₂CH₂CH₂-φ]-benzyl,
4-[-NHC(O)NHCH₂CH₂-φ]-benzyl,
—CH₂C(O)NH(CH₂)₄φ,
4-[φ(CH₂)₄O-]-benzyl,
4-[-C≡C-φ-4′φ]-benzyl,
4-[-C—C—CH₂—O—S(O)₂-4′—CH₃-φ]-benzyl,
4-[-C≡C—CH₂NHC(O)NH₂]-benzyl,
4-[-C≡C—CH₂—O-4′—COOCH₂CH₃-φ]-benzyl,
4-[-C≡C—CH(NH₂)-cyclohexyl]-benzyl,
—(CH₂)₄NHC(O)CH₂-3-indolyl,
—(CH₂)₄NHC(O)CH₂CH₂-3-indolyl,
—(CH₂)₄NHC(O)-3-(5-methoxyindolyl),
—(CH₂)₄NHC(O)-3-(1-methylindolyl),
—(CH₂)₄NHC(O)-4-(-SO₂(CH₃)-φ),
—(CH₂)₄NHC(O)-4-(C(O)CH₃)-phenyl,
—(CH₂)₄NHC(O)-4-fluorophenyl,
—(CH₂)₄NHC(O)CH₂O-4-fluorophenyl,
4-[-C≡C-(2-pyridyl)]benzyl,
4-[-C≡C—CH₂—O-phenyl]benzyl,
4-[-C≡C—CH₂OCH₃]benzyl,
4-[-C≡C-(3-hydroxyphenyl)]benzyl,
4-[-C≡C—CH₂—O-4′-(-C(O)OC₂H₅)phenyl]benzyl,
4-[-C≡C—CH₂CH(C(O)OCH₃)₂]benzyl,
4-[-C≡C—CH₂NH-(4,5-dihydro-4-oxo-5-phenyl-oxazol-2-yl),
3-aminobenzyl,
4-[-C≡C—CH₂CH(NHC(O)CH₃)C(O)OH]-benzyl,
—CH₂C(O)NHCH(CH₃)φ,
—CH₂C(O)NHCH₂-(4-dimethylamino)-φ,
—CH₂C(O)NHCH₂-4-nitrophenyl,
—CH₂CH₂C(O)N(CH₃)CH₂-φ,
—CH₂CH₂C(O)NHCH₂CH₂-(N-methyl)-2-pyrrolyl,
—CH₂CH₂C(O)NHCH₂CH₂CH₂CH₃,
—CH₂CH₂C(O)NHCH₂CH₂-3-indolyl,
—CH₂C(O)N(CH₃)CH₂phenyl,
—CH₂C(O)NH(CH₂)₂-(N-methyl)-2-pyrrolyl,
—CH₂C(O)NHCH₂CH₂CH₂CH₃,
—CH₂C(O)NHCH₂CH₂-3-indolyl,
—(CH₂)₂C(O)NHCH(CH₃)φ,
—(CH₂)₂C(O)NHCH₂-4-dimethylaminophenyl,
—(CH₂)₂C(O)NHCH₂-4-nitrophenyl,
—CH₂C(O)NH-4-[-NHC(O)CH₃-phenyl],
—CH₂C(O)NH-4-pyridyl,
—CH₂C(O)NH-4-[dimethylaminophenyl],
—CH₂C(O)NH-3-methoxyphenyl,
—CH₂CH₂C(O)NH-4-chlorophenyl,
—CH₂CH₂C(O)NH-2-pyridyl,
—CH₂CH₂C(O)NH-4-methoxyphenyl,
—CH₂CH₂C(O)NH-3-pyridyl,
4-[(CH₃)₂NCH₂CH₂O-]benzyl,
—(CH₂)₃NHC(NH)NH—SO₂-4-methylphenyl,
4-[(CH₃)₂NCH₂CH₂O-]benzyl,
—(CH₂)₄NHC(O)NHCH₂CH₃,
—(CH₂)₄NHC(O)NH-phenyl,
—(CH₂)₄NHC(O)NH-4-methoxyphenyl,
4-[4′-pyridyl-C(O)NH-]benzyl,
4-[3′-pyridyl-C(O)NH-]benzyl,
4-[-NHC(O)NH-3′-methylphenyl]benzyl,
4-[-NHC(O)CH₂NHC(O)NH-3′-methylphenyl]benzyl,
4-[-NHC(O)-(2′,3′-dihydroindol-2-yl)]benzyl,
4-[-NHC(O)-(2′,3′-dihydro-N-Boc-indol-2-yl)]benzyl,
p-[—OCH₂CH₂-1′-(4′-pyrimidinyl)-piperazinyl]benzyl,
4-[—OCH₂CH₂-(1′-piperidinyl)benzyl,
4-[—OCH₂CH₂-(1′-pyrrolidinyl)]benzyl,
4-[—OCH₂CH₂CH₂-(1′-piperidinyl)]benzyl-,
—CH₂-3-(1,2,4-triazolyl),
4-[—OCH₂CH₂CH₂-4-(3′-chlorophenyl)-piperazin-1-yl]benzyl,
4-[—OCH₂CH₂N(φ)CH₂CH₃]benzyl,
4-[—OCH₂-3′-(N-Boc)-piperidinyl]benzyl,
4-[di-n-pentylamino]benzyl,
4-[n-pentylamino]benzyl,
4-[di-iso-propylamino-CH₂CH₂O-]benzyl,
4-[—OCH₂CH₂-(N-morpholinyl)]benzyl,
4-[—O—(3′-(N-Boc)-piperidinyl]benzyl,
4-[—OCH₂CH(NHBoc)CH₂cyclohexyl]benzyl,
p-[OCH₂CH₂-(N-piperidinyl]benzyl,
4-[—OCH₂CH₂CH₂-(4-m-chlorophenyl)-piperazin-1-yl]benzyl,
4-[—OCH₂CH₂-(N-homopiperidinyl)benzyl,
4-[-NHC(O)-3′-(N-Boc)-piperidinyl]benzyl,
4-[—OCH₂CH₂N(benzyl)₂]benzyl,
—CH₂-2-thiazolyl,
3-hydroxybenzyl,
4-[—OCH₂CH₂CH₂N(CH₃)₂]benzyl,
4-[-NHC(S)NHCH₂CH₂-(N-morpholino)]benzyl,
4-[—OCH₂CH₂N(C₂H₅)₂]benzyl,
4-[—OCH₂CH₂CH₂N(C₂H₅)₂]benzyl,
4-[CH₃(CH₂)₄NH-]benzyl,
4-[N-n-butyl,N-n-pentylamino-]benzyl,
4-[-NHC(O)-4′-piperidinyl]benzyl,
4-[-NHC(O)CH(NHBoc)(CH₂)₄NHCbz]benzyl,
4-[-NHC(O)-(1′,2′,3′,4′-tetrahydro-N-Boc-isoquinolin-1′-yl]benzyl,
p-[—OCH₂CH₂CH₂-1′-(4′-methyl)-piperazinyl]benzyl,
—(CH₂)₄NH-Boc,
3-[—OCH₂CH₂CH₂N(CH₃)₂]benzyl,
4-[—OCH₂CH₂CH₂N(CH₃)₂]benzyl,
3-[—OCH₂CH₂-(1′-pyrrolidinyl)]benzyl,
4-[—OCH₂CH₂CH₂N(CH₃)benzyl]benzyl,
4-[-NHC(S)NHCH₂CH₂CH₂-(N-morpholino)]benzyl,
4-[—OCH₂CH₂-(N-morpholino)]benzyl,
4-[-NHCH₂-(4′-chlorophenyl)]benzyl,
4-[-NHC(O)NH-(4′-cyanophenyl)]benzyl,
4-[—OCH₂COOH]benzyl,
4-[—OCH₂COO-t-butyl]benzyl,
4-[-NHC(O)-5′-fluoroindol-2-yl]benzyl,
4-[-NHC(S)NH(CH₂)₂-1-piperidinyl]benzyl,
4-[-N(SO₂CH₃)(CH₂)₃—N(CH₃)₂]benzyl,
4-[-NHC(O)CH₂CH(C(O)OCH₂φ)-NHCbz]benzyl,
4-[-NHS(O)₂CF₃]benzyl,
3-[—O—(N-methylpiperidin-4′-yl]benzyl,
4-[-C(═NH)NH₂]benzyl,
4-[-NHSO₂—CH₂Cl]benzyl,
4-[-NHC(O)-(1′,2′,3′,4′-tetrahydroisoquinolin-2′-yl]benzyl,
4-[-NHC(S)NH(CH₂)₃-N-morpholino]benzyl,
4-[-NHC(O)CH(CH₂CH₂CH₂CH₂NH₂)NHBoc]benzyl,
4-[-C(O)NH₂]benzyl,
4-[-NHC(O)NH-3′-methoxyphenyl]benzyl,
4-[—OCH₂CH₂-indol-3′-yl]benzyl,
4-[—OCH₂C(O)NH-benzyl]benzyl,
4-[—OCH₂C(O)O-benzyl]benzyl,
4-[—OCH₂C(O)OH]benzyl,
4-[—OCH₂-2′-(4′,5′-dihydro)imidazolyl]benzyl,
—CH₂C(O)NHCH₂-(4-dimethylamino)phenyl,
—CH₂C(O)NHCH₂-(4-dimethylamino)phenyl,
4-[-NHC(O)-L-2′-pyrrolidinyl-N—SO₂-4′-methylphenyl]benzyl,
4-[-NHC(O)NHCH₂CH₂CH₃]benzyl,
4-aminobenzyl]benzyl,
4-[—OCH₂CH₂-1-(4-hydroxy-4-(3-methoxypyrrol-2-yl)-piperazinyl]benzyl,
4-[—O—(N-methylpiperidin-4′-yl)]benzyl,
3-methoxybenzyl,
4-[-NHC(O)-piperidin-3′-yl]benzyl,
4-[-NHC(O)-pyridin-2′-yl]benzyl,
4-[-NHCH₂-(4′-chlorophenyl)]benzyl,
4-[-NHC(O)-(N-(4′—CH₃-φ-SO₂)-L-pyrrolidin-2′-yl)]benzyl,
4-[-NHC(O)NHCH₂CH₂-φ]benzyl,
4-[—OCH₂C(O)NH₂]benzyl,
4-[—OCH₂C(O)NH-t-butyl]benzyl,
4-[—OCH₂CH₂-1-(4-hydroxy-4-phenyl)-piperidinyl]benzyl,
4-[-NHSO₂—CH═CH₂]benzyl,
4-[-NHSO₂—CH₂CH₂Cl]benzyl,
—CH₂C(O)NHCH₂CH₂N(CH₃)₂,
4-[(1′—Cbz-piperidin-4′-yl)C(O)NH-]benzyl,
4-[(1′-Boc-piperidin-4′-yl)C(O)NH-]benzyl,
4-[(2′-bromophenyl)C(O)NH-]benzyl,
4-[-NHC(O)-pyridin-4′-yl]benzyl,
4-[(4′-(CH₃)₂NC(O)O-)phenyl)-C(O)NH-]benzyl,
4-[-NHC(O)-1′-methylpiperidin-4′-yl-]benzyl,
4-(dimethylamino)benzyl,
4-[-NHC(O)-(1′-N-Boc)-piperidin-2′-yl]benzyl,
3-[-NHC(O)-pyridin-4′-yl]benzyl,
4-[(tert-butyl-O(O)CCH₂—O-benzyl)-NH-]benzyl,
[BocNHCH₂C(O)NH-]butyl,
4-benzylbenzyl,
2-hydroxyethyl,
4-[(Et)₂NCH₂CH₂CH₂NHC(S)NH-]benzyl,
4-[(1′-Boc-4′-hydroxypyrrolidin-2′-yl)C(O)NH-]benzyl,
4-[(φCH₂CH₂CH₂NHC(S)NH-]benzyl,
4-[(perhydroindolin-2′-yl)C(O)NH-]benzyl,
2-[4-hydroxy-4-(3-methoxythien-2-yl)piperidin-1-yl]ethyl,
4-[(1′-Boc-perhydroindolin-2′-yl)-C(O)NH-]benzyl,
4-[N-3-methylbutyl-N-trifluoromethanesulfonyl)amino]benzyl,
4-[N-vinylsulfonyl)amino]benzyl,
4-[2-(2-azabicyclo[3.20.2]octan-2-yl)ethyl-O-]benzyl,
4-[4′-hydroxypyrrolidin-2′-yl)C(O)NH-]benzyl,
4-(φNHC(S)NH)benzyl,
4-(EtNHC(S)NH)benzyl,
4-(φCH₂NHC(S)NH)benzyl,
3-[(1′-Boc-piperidin-2′-yl)C(O)NH-]benzyl,
3-[piperidin-2′-yl-C(O)NH-]benzyl,
4-[(3′-Boc-thiazolidin-4′-yl)C(O)NH-]benzyl,
4-(pyridin-3′-yl-NHC(S)NH)benzyl,
4-(CH₃—NHC(S)NH)benzyl,
4-(H₂NCH₂CH₂CH₂C(O)NH)benzyl,
4-(BocHNCH₂CH₂CH₂C(O)NH)benzyl,
4-(pyridin-4′-yl-CH₂NH)benzyl,
4-[(N,N-di(4-N,N-dimethylamino)benzyl)amino]benzyl,
4-[(1—Cbz-piperidin-4-yl)C(O)NH-]butyl,
4-[(φCH₂OCH₂(BocHN)CHC(O)NH]benzyl,
4-[(piperidin-4′-yl)C(O)NH-]benzyl,
4-[(pyrrolidin-2′-yl)C(O)NH-]benzyl,
4-(pyridin-3′-yl-C(O)NH)butyl,
4-(pyridin-4′-yl-C(O)NH)butyl,
4-(pyridin-3′-yl-C(O)NH)benzyl,
4-[CH₃NHCH₂CH₂CH₂C(O)NH-]benzyl,
4-[CH₃N(Boc)CH₂CH₂CH₂C(O)NH-]benzyl,
4-(aminomethyl)benzyl,
4-[(φCH₂OCH₂(H₂N)CHC(O)NH]benzyl,
4-[(1′,4′-di(Boc)piperazin-2′-yl)-C(O)NH-]benzyl,
4-[(piperazin-2′-yl)-C(O)NH-]benzyl,
4-[(N-toluenesulfonylpyrrolidin-2′-yl)C(O)NH-]butyl,
4-[-NHC(O)-4′-piperidinyl]butyl,
4-[-NHC(O)-1′-N-Boc-piperidin-2′-yl]benzyl,
4-[-NHC(O)-piperidin-2′-yl]benzyl,
4-[(1′-N-Boc-2′,3′-dihydroindolin-2′-yl)-C(O)NH]benzyl,
4-(pyridin-3′-yl-CH₂NH)benzyl,
4-[(piperidin-1′-yl)C(O)CH₂—O-]benzyl,
4-[(CH₃)₂CH)₂NC(O)CH₂—O-]benzyl,
4-[HO(O)C(Cbz-NH)CHCH₂CH₂—C(O)NH-]benzyl,
4-[(φCH₂O(O)C(Cbz-NH)CHCH₂CH₂—C(O)NH-]benzyl,
4-[-NHC(O)-2′-methoxyphenyl]benzyl,
4-[(pyrazin-2′-yl)C(O)NH-]benzyl,
4-[HO(O)C(NH₂)CHCH₂CH₂—C(O)NH-]benzyl,
4-(2′-formyl-1′,2′,3′,4′-tetrahydroisoquinolin-3′-yl-CH₂NHbenzyl,
N-Cbz-NHCH₂—,
4-[(4′-methylpiperazin-1′-yl)C(O)O-]benzyl,
4-[CH₃(N-Boc)NCH₂C(O)NH-]benzyl,
4-[-NHC(O)-(1′,2′,3′,4′-tetrahydro-N-Boc-isoquinolin-3′-yl]-benzyl,
4-[CH₃NHCH₂C(O)NH-]benzyl,
(CH₃)₂NC(O)CH₂—,
4-(N-methylacetamido)benzyl,
4-(11,2′,3′,4′-tetrahydroisoquinolin-3′-yl-CH₂NHbenzyl,
4-[(CH₃)₂NHCH₂C(O)NH-]benzyl,
(1-toluenesulfonylimidizol-4-yl)methyl,
4-[(1′-Boc-piperidin-4′-yl)C(O)NH-]benzyl,
4-trifluoromethylbenzyl,
4-[(2′-bromophenyl)C(O)NH-]benzyl,
4-[(CH₃)₂NC(O)NH-]benzyl,
4-[CH₃₀C(O)NH-]benzyl,
4-[(CH₃)₂NC(O)O-]benzyl,
4-[(CH₃)₂NC(O)N(CH₃)-]benzyl,
4-[CH₃₀C(O)N(CH₃)-]benzyl,
4-(N-methyltrifluoroacetamido)benzyl,
4-[(1′-methoxycarbonylpiperidin-4′-yl)C(O)NH-]benzyl,
4-[(4′-phenylpiperidin-4′-yl)C(O)NH-]benzyl,
4-[(4′-phenyl-1′-Boc-piperidin-4′-yl)-C(O)NH-]benzyl,
4-[(piperidin-4′-yl)C(O)O-]benzyl, 4-[(1′-methylpiperidin-4′-yl)-O-]benzyl,
4-[(1′-methylpiperidin-4′-yl)C(O)O-]benzyl,
4-[(4′-methylpiperazin-1′-yl)C(O)NH-]benzyl,
3-[(CH₃)₂NC(O)O-]benzyl,
4-[(4′-phenyl-1′-Boc-piperidin-4′-yl)-C(O)O-]benzyl,
4-(N-toluenesulfonylamino)benzyl,
4-[(CH₃)₃CC(O)NH-]benzyl,
4-[(morpholin-4′-yl)C(O)NH-]benzyl,
4-[(CH₃CH₂)₂NC(O)NH-]benzyl,
4-[-C(O)NH-(4′-piperidinyl)]benzyl,
4-[(2′-trifluoromethylphenyl)C(O)NH-]benzyl,
4-[(2′-methylphenyl)C(O)NH-]benzyl,
4-[(CH₃)₂NS(O)₂O-]benzyl,
4-[(pyrrolidin-2′-yl)C(O)NH-]benzyl,
4-[-NHC(O)-piperidin-1′-yl]benzyl,
4-[(thiomorpholin-4′-yl)C(O)NH-]benzyl,
4-[(thiomorpholin-4′-yl sulfone)-C(O)NH-]benzyl,
4-[(morpholin-4′-yl)C(O)O-]benzyl,
3-nitro-4-(CH₃OC(O)CH₂Obenzyl,
(2-benzoxazolinon-6-yl)methyl-,
(2H-1,4-benzoxazin-3 (4H)-one-7-yl)methyl-,
4-[(CH₃)₂NS(O)₂NH-]benzyl,
4-[(CH₃)₂NS(O)₂N(CH₃)-]benzyl,
4-[(thiomorpholin-4′-yl)C(O)O-]benzyl,
4-[(thiomorpholin-4′-yl sulfone)-C(O)O-]benzyl,
4-[(piperidin-1′-yl)C(O)O-]benzyl,
4-[(pyrrolidin-1′-yl)C(O)O-]benzyl,
4-[(4′-methylpiperazin-1′-yl)C(O)O-]benzyl,
4-[(2′-methylpyrrolidin-1′-yl)-,
(pyridin-4-yl)methyl-,
4-[(piperazin-4′-yl)-C(O)O-]benzyl,
4-[(1′-Boc-piperazin-4′-yl)-C(O)O-]benzyl,
4-[(4′-acetylpiperazin-1′-yl)C(O)O-]benzyl,
p-[(4′-methanesulfonylpiperazin-1′-yl)-benzyl,
3-nitro-4-[(morpholin-4′-yl)-C(O)O-]benzyl,
4-{[(CH₃)₂NC(S)]₂N-}benzyl,
N-Boc-2-aminoethyl-,
4-[(1,1-dioxothiomorpholin-4-yl)-C(O)O-]benzyl,
4-[(CH₃)₂NS(O)₂-]benzyl,
4-(imidazolid-2′-one-1′-yl)benzyl,
4-[(piperidin-1′-yl)C(O)O-]benzyl,
1-N-benzyl-imidazol-4-yl-CH₂—,
3,4-dioxyethylenebenzyl (i.e., 3,4-ethylenedioxybenzyl),
3,4-dioxymethylenebenzyl (i.e., 3,4-methylenedioxybenzyl),
4-[-N(SO₂)(CH₃)CH₂CH₂CH₂N(CH₃)₂]benzyl,
4-(3′-formylimidazolid-2′-one-1′-yl)benzyl,
4-[NHC(O)CH(CH₂CH₂CH₂CH₂NH₂)NHBoc]benzyl,
[2′-[4″-hydroxy-4″-(3′″-methoxythien-2′″-yl)piperidin-2″-yl]ethoxy]benzyl, and
p-[(CH₃)₂NCH₂CH₂N(CH₃)C(O)O-]benzyl.

Preferably, R⁵ in the above compounds is selected from the group consisting of alkyl, substituted alkyl, aryl, substituted aryl, heterocyclic, substituted heterocylic, heteroaryl and substituted heteroaryl. Even more preferably R⁵ is selected from the group consisting of 4-methylphenyl, methyl, benzyl, n-butyl, n-hexyl, 4-chlorophenyl, 1-naphthyl, 2-naphthyl, 4-methoxyphenyl, phenyl, 2,4,6-trimethylphenyl, 2-(methoxycarbonyl)phenyl, 2-carboxyphenyl, 3,5-dichlorophenyl, 4-trifluoromethylphenyl, 3,4-dichlorophenyl, 3,4-dimethoxyphenyl, 4-(CH₃C(O)NH-)phenyl, 4-trifluoromethoxyphenyl, 4-cyanophenyl, isopropyl, 3,5-di-(trifluoromethyl)phenyl, 4-t-butylphenyl, 4-t-butoxyphenyl, 4-nitrophenyl, 2-thienyl, 1-N-methyl-3-methyl-5-chloropyrazol-4-yl, phenethyl, 1-N-methylimidazol-4-yl, 4-bromophenyl, 4-amidinophenyl, 4-methylamidinophenyl, 4-[CH₃SC(═NH)]phenyl, 5-chloro-2-thienyl, 2,5-dichloro-4-thienyl, 1-N-methyl-4-pyrazolyl, 2-thiazolyl, 5-methyl-1,3,4-thiadiazol-2-yl, 4-[H₂NC(S)]phenyl, 4-aminophenyl, 4-fluorophenyl, 2-fluorophenyl, 3-fluorophenyl, 3,5-difluorophenyl, pyridin-3-yl, pyrimidin-2-yl, 4-(3′-dimethylamino-n-propoxy)-phenyl, and 1-methylpyrazol-4-yl.

Preferably, R¹³ in the above compounds is selected from hydrogen or C₁₋₆ alkyl; more preferably, hydrogen or C₁₋₃ alkyl; and still more preferably, hydrogen or methyl.

In a preferred embodiment, R¹⁴ in the above compounds is preferably hydrogen and R¹⁵ is preferably C₁₋₁₀ alkyl or Cy-C₁₋₁₀ alkyl, wherein alkyl is optionally substituted with one to four substituents selected from phenyl and R^{x}, and Cy is optionally substituted with one to four substituents independently selected from R^{y}, or R¹⁴ and R¹⁵ and the carbon to which they are attached together from a 3-7 membered mono- or bicyclic carbon only ring. For the purpose of R¹⁵, Cy is preferably aryl, more preferably phenyl. In a preferred embodiment, R¹⁵ is phenyl-C₁₋₃ alkyl, wherein phenyl is optionally substituted with one or two groups selected from R^{y}. Additional preferred embodiments for R¹⁴ and R¹⁵ are disclosed in International Patent Application Publication No. WO 98/53814, which application is incorporated herein by reference in its entirety.

In a preferred embodiment of the above compounds, R¹⁶ is substituted amino; R¹⁷ and/or R²⁰ are hydrogen; and R¹⁸ and/or R²¹ are alkyl, substituted alkyl, aryl or substituted aryl.

In a preferred embodiment, R²³ in the above compounds is hydrogen. Preferably, R²⁴ in the above compounds is Ar¹—Ar²—C₁₀ alkyl wherein Ar¹ and Ar² are optionally substituted with from 1 to 4 groups independently selected from R^{b} and R²⁵ is hydrogen. More preferably, R²⁴ is Ar¹—Ar²—C₁₋₃ alkyl wherein Ar¹ and Ar² are optionally substituted with from 1 to 4 groups independently selected from R^{b}; still more preferably, R²⁴ is —CH₂—Ar²—Ar¹ and R²⁵ is hydrogen. Additional preferred embodiments are disclosed in International Patent Application Publication No. WO 98/53817, which application is incorporated herein by reference in its entirety.

Preferably, R³ and R^{3′}, or R¹⁴ and R¹⁵, or R²⁴ and R²⁵ are derived from L-amino acids or other similarly configured starting materials. Alternatively, racemic mixtures can be used.

Preferred compounds include those set forth in the Tables below:

TABLE 2

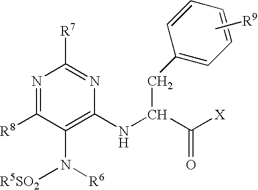

| | | | | | |
|---|---|---|---|---|---|
| R⁵ | R⁶ | R⁷ | R⁸ | R⁹ | X |

| | | | | | |
|---|---|---|---|---|---|
| 4-CH₃—Ph— | H— | H— | H— | 4-(CH₃)₂NC(O)O— | —OC(CH₃)₃ |
| 4-CH₃—Ph— | H— | H— | H— | 4-(CH₃)₂NC(O)O— | —OH |
| 4-CH₃—Ph— | CH₃— | H— | H— | 4-(CH₃)₂NC(O)O— | —OC(CH₃)₃ |
| 4-CH₃—Ph— | CH₃— | H— | H— | 4-(CH₃)₂NC(O)O— | —OH |
| 4-CH₃—Ph— | 4-CH₃—Ph— | H— | H— | 4-(CH₃)₂NC(O)O— | —OH |
| 1-CH₃— | CH₃— | H— | H— | 4-(CH₃)₂NC(O)O— | —OH |
| pyrazol-4-yl | | | | | |
| 4-CH₃—Ph— | CH₃— | H— | H— | 4-(CH₃)₂NC(O)O— | —OCH(CH₃)₂ |
| 3-pyridyl- | CH₃— | H— | H— | 4-(CH₃)₂NC(O)O— | —OC(CH₃)₃ |
| 1-(n- | CH₃— | H— | H— | 4-(CH₃)₂NC(O)O— | —OC(CH₃)₃ |
| C₄H₉) | | | | | |
| pyrazol-4-yl- | | | | | |
| 4-CH₃—Ph— | CH₃— | H— | H— | H— | —OH |
| 1-(n- | CH₃— | H— | H— | 4-(CH₃)₂NC(O)O— | —OH |
| C₄H₉)— | | | | | |
| pyrazol-4-yl | | | | | |
| 3-pyridyl- | CH₃— | H— | H— | 4-(CH₃)₂NC(O)O— | —OH |
| 4-CH₃—Ph— | CH₃— | (CH₃)₂N— | H— | H— | —OH |
| 1-CH₃— | CH₃— | H— | H— | 4-(CH₃)₂NC(O)O— | —OCH(CH₃)₂ |
| pyrazol-4-yl | | | | | |
| 3-pyridyl- | CH₃— | H— | H— | 4-(1-CH₃-piperazin- | —OCH(CH₃)₂ |
| | | | | 4-yl)C(O)O- | |
| 3-pyridyl- | CH₃— | H— | H— | 4-(1-CH₃-piperazin- | —OC(CH₃)₃ |
| | | | | 4-yl)C(O)O- | |
| 3-pyridyl- | CH₃— | H— | H— | 4-(1-CH₃-piperazin- | —OH |
| | | | | 4-yl)-C(O)O- | |

Ph = phenyl

TABLE 3

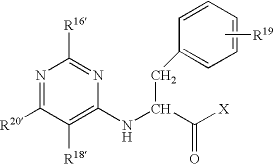

| R^{16′} | R^{20′} | R^{18′} | R¹⁹ | X |
|---|---|---|---|---|
| Cl— | H— | NO₂— | 4-(CH₃)₂NC(O)O— | —OH |
| H— | H— | PhCH₂O— | H— | —OH |
| H— | H— | PhCH₂O— | 4-(CH₃)₂NC(O)O— | —OH |
| H— | H— | Ph— | 4-(CH₃)₂NC(O)O— | —OH |
| H— | H— | 3-NO₂—Ph— | 4-(CH₃)₂NC(O)O— | —OH |
| H— | H— | 3-pyridyl- | 4-(CH₃)₂NC(O)O— | —OH |
| H— | H— | 2-PhCH₂CH₂— | 4-(CH₃)₂NC(O)O— | —OH |
| H— | H— | 2-CH₃—Ph— | 4-(CH₃)₂NC(O)O— | —OH |
| H— | H— | (CH₃)₂NC(O)— | 4-(CH₃)₂NC(O)O— | —OH |
| | | (CH₂)₂— | | |
| H— | Ph— | H— | 4-(CH₃)₂NC(O)O— | —OH |
| H— | 2-CF₃— | H— | 4-(CH₃)₂NC(O)O— | —OH |
| | Ph- | | | |
| H— | 2- | H— | 4-(CH₃)₂NC(O)O— | —OH |
| | HOC | | | |
| | H₂Ph- | | | |
| H— | H— | CF₃CH₂— | 4-(CH₃)₂NC(O)O— | —OH |
| H— | H— | PhCH₂— | 4-(CH₃)₂NC(O)O— | —OH |
| H— | H— | 2-CH₃—Ph— | 4-(CH₃)₂NC(O)O— | —OCH(CH₃)₂ |
| H— | H— | 2-PhCH₂CH₂— | 4-(CH₃)₂NC(O)O— | —OCH(CH₃)₂ |
| H— | H— | 2-PhCH₂CH₂— | H— | —OCH(CH₃)₂ |
| cyclohexyl | H— | H— | 4-(CH₃)₂NC(O)O— | —OH |
| —(CH₃)N— | | | | |
| H— | H— | CH₃CH₂CH₂— | 4-(CH₃)₂NC(O)O— | —OH |
| H— | H— | 2-CH₃O—Ph— | 4-(CH₃)₂NC(O)O— | —OH |
| H— | H— | 2-F—Ph— | 4-(CH₃)₂NC(O)O— | —OH |
| (CH₃)₂CH | H— | 2-CH₃—Ph— | 4-(CH₃)₂NC(O)O— | —OH |
| —(CH₃)N— | | | | |
| (CH₃)₂CH | H— | 2-CH₃—Ph— | 4-(CH₃)₂NC(O)O— | —OH |
| —NH— | | | | |
| (CH₃)₂CH | H— | 2-CH₃—Ph— | 4-(CH₃)₂NC(O)O— | —OH |
| CH₂— | | | | |
| (CH₃)N— | | | | |
| CH₃CH₂C | H— | 2-CH₃—Ph— | 4-(CH₃)₂NC(O)O— | —OH |
| H₂— | | | | |
| (CH₃)N— | | | | |
| (CH₃)₂N— | H— | 2-CH₃—Ph— | 4-(CH₃)₂NC(O)O— | —OH |
| cyclohexyl | H— | 3-pyridyl- | 4-(CH₃)₂NC(O)O— | —OH |
| —(CH₃)N— | | | | |
| H— | H— | 2-PhCF₂CH₂— | 4-(CH₃)₂NC(O)O— | —OH |
| H— | Cl— | 2-PhCF₂CH₂— | 4-(CH₃)₂NC(O)O— | —OH |
| (HOCH₂C | H— | H— | 4-(CH₃)₂NC(O)O— | —OH |
| H₂)₂N— | | | | |
| (HOCH₂C | H— | 2-CH₃—Ph— | 4-(CH₃)₂NC(O)O— | —OH |
| H₂)₂N— | | | | |
| Ph(CH₃)N— | H— | 2-CH₃—Ph— | 4-(CH₃)₂NC(O)O— | —OH |
| (CH₃)₂CH | H— | 2-CH₃—Ph— | 4-(CH₃)₂NC(O)O— | —OH |
| O— | | | | |
| (CH₃)₂CH | H— | 2-CH₃—Ph— | 4-(CH₃)₂NC(O)O— | —OH |
| CH₂— | | | | |
| CH₂(CH₃) | | | | |
| N— | | | | |
| CH₃NH— | H— | 2-CH₃—Ph— | 4-(CH₃)₂NC(O)O— | —OH |
| 2-CH₃—Ph— | H— | 2-CH₃—Ph— | 4-(CH₃)₂NC(O)O— | —OH |
| HOCH₂C | H— | 2-CH₃—Ph— | 4-(CH₃)₂NC(O)O— | —OH |
| H₂— | | | | |
| (CH3)N— | | | | |
| cyclohexyl | H— | 2-CH₃—Ph— | 4-(CH₃)₂NC(O)O— | —OH |
| —NH— | | | | |
| 1-CH₃- | H— | 2-CH₃—Ph— | 4-(CH₃)₂NC(O)O— | —OH |
| piperidin- | | | | |
| 4-yl- | | | | |
| (CH₃)N— | | | | |
| (CH₃)₂CH— | H— | 2-CH₃—Ph— | 4-(CH₃)₂NC(O)O— | —OH |
| (CH₃CH₂—)N— | | | | |
| H— | H— | 2,4,6-tri-CH₃—Ph— | 4-(CH₃)₂NC(O)O— | —OH |
| H— | H— | (CH₃)₂CH— | 4-(CH₃)₂NC(O)O— | —OH |
| CH₃(CH₂)₃— | H— | 2-CH₃—Ph— | 4-(CH₃)₂NC(O)O— | —OH |
| (CH₃)N— | | | | |
| CH₃CH₂CH₂— | H— | 2-CH₃—Ph— | 4-(CH₃)₂NC(O)O— | —OH |
| (CH₃CH₂—)N— | | | | |
| (CH₃CH₂)₂N— | H— | 2-CH₃—Ph— | 4-(CH₃)₂NC(O)O— | —OH |
| CH₃CH₂— | H— | 2-CH₃—Ph— | 4-(CH₃)₂NC(O)O— | —OH |
| (CH₃)N— | | | | |
| H— | H— | cyclohexyl- | 4-(CH₃)₂NC(O)O— | —OH |
| (furan-2-yl)ch₂— | H— | 2-CH₃—Ph— | 4-(CH₃)₂NC(O)O— | —OH |
| (CH₃)N— | | | | |
| 4-Cl—Ph— | H— | 2-CH₃—Ph— | 4-(CH₃)₂NC(O)O— | —OH |
| (CH₃)N— | | | | |
| H— | H— | thien-3-yl- | 4-(CH₃)₂NC(O)O— | —OH |
| H— | H— | thien-2-yl- | 4-(CH₃)₂NC(O)O— | —OH |
| HOCH₂CH₂— | H— | 2-F—Ph— | 4-(CH₃)₂NC(O)O— | —OH |
| (CH₃)N— | | | | |
| H— | H— | piperidin-1-y- | 4-(CH₃)₂NC(O)O— | —OH |
| H— | H— | (CH₃CH₂CH₂)₂—CH— | 4-(CH₃)₂NC(O)O— | —OH |
| cyclobutyl | H— | 2-CH₃—Ph— | 4-(CH₃)₂NC(O)O— | —OH |
| H— | H— | 2-HOCH₂—Ph— | 4-(CH₃)₂NC(O)O— | —OH |
| H— | H— | 2,6-di-F—Ph— | 4-(CH₃)₂NC(O)O— | —OH |
| H— | H— | 2,4-di-CH₃O— | 4-(CH₃)₂NC(O)O— | —OH |
| | | pyrimidin-5-yl | | |
| cyclohexyl | H— | 2-CH₃—Ph— | 4-(CH₃)₂NC(O)O— | —OH |
| —(CH₃)N— | | | | |
| H— | H— | 2-CF₃—Ph— | 4-(CH₃)₂NC(O)O— | —OH |
| cyclohexyl | H— | 2-CH₃O—Ph— | 2,6-di-CH₃O—Ph— | —OH |
| —(CH₃)N— | | | | |
| (CH₃)₂CH | H— | 2-F—Ph— | 2,6-di-CH₃O—Ph— | —OH |
| —(CH_{3L )N—} | | | | |
| (CH₃)₂CH | H— | 2-F—Ph— | 2-CH₃O—Ph— | —OH |
| —(CH₃)N— | | | | |
| cyclohexyl | H— | 2,6-di-F—Ph— | 2,6-di-F—Ph— | —OH |
| —(CH₃)N— | | | | |
| cyclohexyl | H— | 2-HOCH₂—Ph— | 2,6-di-CH₃O—Ph— | —OH |
| —(CH₃)N— | | | | |
| (HOCH₂CH₂)₂N— | H— | 2,4,6-tri-CH₃—Ph— | 2,6-di-CH₃O—Ph— | —OH |
| cyclohexyl | H— | 2-CF₃—Ph— | 2-NC—Ph— | —OH |
| —(CH₃)N— | | | | |
| cyclohexyl | H— | thien-3-yl- | 2,6-di-CH₃O—Ph— | —OH |
| —(CH₃)N— | | | | |
| cyclohexyl | H— | thien-2-yl- | 4-CF₃—Ph— | —OH |
| —(CH₃)N— | | | | |
| cyclohexyl | H— | 3-pyridyl- | 2,6-di-CH₃O—Ph— | —OH |
| —(CH₃)N— | | | | |
| cyclohexyl | H— | 2-NO₂—Ph— | 2,6-di-CH₃O—Ph— | —OH |
| —(CH₃)N— | | | | |
| cyclohexyl | H— | 2,6-di-Cl—Ph— | 2,6-di-CH₃O—Ph— | —OH |
| —(CH₃)N— | | | | |
| cyclohexyl | H— | 4-pyridyl- | 3-HOCH₂—Ph— | —OH |
| —(CH₃)N— | | | | |
| (CH₃)₂CH— | H— | 2,6-di-CH₃O—Ph— | 2,6-di-CH₃O—Ph— | —OH |
| (CH₃CH₂—)N— | | | | |
| cyclohexyl | H— | 2,6-di-Cl—Ph— | 2,6-di-CH₃O—Ph— | —OH |
| —(CH₃)N— | | | | |
| CH₃CH₂— | H— | 2,4,6-tri-CH₃—Ph— | 2-NC—Ph— | —OH |
| (CH3)N— | | | | |
| (CH₃)₂CH | H— | 2,4,6-tri-CH₃—Ph— | 3-pyridyl- | —OH |
| —(CH₃)N— | | | | |
| (HOCH₂HCH₂)₂N— | H— | 2,4,6-tri-CH₃—Ph— | 2-NC—Ph— | —OH |
| 1-CH₃- | H— | 2-NC—Ph— | 2,6-di-F—Ph— | —OH |
| piperidin- | | | | |
| 4-yl- | | | | |
| (CH₃)N— | | | | |
| (CH₃)₂CH— | H— | 2,4,6-tri-CH₃—Ph— | 2-CH₃—Ph— | —OH |
| (CH₃CH₂—)N— | | | | |
| 4-Cl—Ph— | H— | 2,4,6-tri-CH₃—Ph— | 2,6-di-CH₃O—Ph— | —OH |
| (CH₃)N— | | | | |
| H— | H— | PhCH₂CH₂—(CH₃)N— | 4-(CH₃)₂NC(O)O— | —OH |
| H— | H— | CH₃(CH₂)₅—(CH₃)N— | 4-(CH₃)₂NC(O)O— | —OH |
| H— | H— | (CH₃)₂CH—(CH₃)N- | 4-(CH₃)₂NC(O)O— | —OH |
| H— | H— | (CH₃)₃C—(CH₃)N— | 4-(CH₃)₂NC(O)O— | —OH |
| H— | H— | (CH₃)₂CH— | 4-(CH₃)₂NC(O)O— | —OH |
| | | (CH₃CH₂—)N— | | |
| H— | H— | 4-pyridyl-CH₂CH₂— | 4-(CH₃)₂NC(O)O— | —OH |
| | | (CH₃)N— | | |
| H— | H— | PhCH₂CH₂—(CH₃)N— | 2,6-di-CH₃O—Ph— | —OH |
| H— | H— | CH₃(CH₂)₅—(CH₃)N— | 2,6-di-CH₃O—Ph— | —OH |
| H— | H— | (CH₃)₂CH—(CH₃)N— | 2,6-di-CH₃O—Ph— | —OH |
| H— | H— | (CH₃)₃C—(CH₃)N— | 2,6-di-CH₃O—Ph— | —OH |
| H— | H— | (CH₃)₂CH— | 2,6-di-CH₃O—Ph— | —OH |
| | | (CH₃CH₂—)N— | | |
| H— | H— | 4-pyridyl-CH₂CH₂— | 2,6-di-CH₃O—Ph— | —OH |
| | | (CH₃)N— | | |
| cyclohexyl | H— | CH₃CH₂— | 4-(CH₃)₂NC(O)O— | —OH |
| —(CH₃)N— | | | | |
| H— | H— | CF₃CH₂— | 2,6-di-CH₃O—Ph— | —OH |
| cyclohexyl | H— | 2-CH₃—Ph— | 2,6-di-CH₃O—Ph— | —OH |
| —(CH₃)N— | | | | |
| H— | H— | 2-F—Ph— | 2,6-di-CH₃O—Ph— | —OH |
| CH₃CH₂CH₂— | H— | 2-CH₃—Ph— | 2,6-di-CH₃O—Ph— | —OH |
| (CH₃)N— | | | | |

Ph = phenyl

TABLE 4

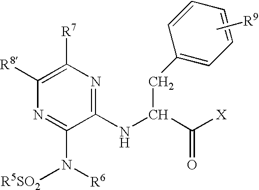

| R⁵ | R⁶ | R^{7′} | R^{8′} | R^{9′} | X |
|---|---|---|---|---|---|
| 4-CH₃—Ph— | CH₃— | H— | H— | 4-(CH₃)₂NC(O)O— | —OH |
| 4-CH₃—Ph— | CH₃— | H— | H— | 4-(CH₃)₂NC(O)O— | —OCH |
| | | | | | (CH₃)₂ |

Ph = phenyl

Accordingly, the following are preferred compounds of formulae IIIa, IIIb, IVa, IVb, IVc, IVd, Va, Vb, Vc, Vd, VIa, VIb, VIc, and VId:
N-(2-chloro-5-nitropyrimidin-4-yl)-L-4-(N,N-dimethylcarbamyloxy)phenylalanine,
N-[5-(N-4-toluenesulfonylamino)pyrimidin-4-yl]-L-4-(N,N-dimethylcarbamyloxy)phenylalanine tert-butyl ester,
N-[5-(N-4-toluenesulfonylamino)pyrimidin-4-yl]-L-4-(N,N-dimethylcarbamyloxy)phenylalanine,
N-[5-(N-methyl-N-4-toluenesulfonylamino)pyrimidin-4-yl]-L-4-(N,N-dimethylcarbamyloxy)phenylalanine tert-butyl ester,
N-[5-(N-methyl-N-4-toluenesulfonylamino)pyrimidin-4-yl]-L-4-(N,N-dimethylcarbamyloxy)phenylalanine,
N-[5-(N,N-di-4-toluenesulfonylamino)pyrimidin-4-yl]-L-4-(N,N-dimethylcarbamyloxy)phenylalanine,
N-[5-[N-(1-N′-methylpyrazol-4-ylsulfonyl)-N-methylamino]pyrimidin-4-yl]-L-4-(N,N-dimethylcarbamyloxy)phenylalanine,
N-[5-(N-methyl-N-4-toluenesulfonylamino)pyrimidin-4-yl]-L-4-(N,N-dimethylcarbamyloxy)phenylalanine isopropyl ester,
N-[5-(N-methyl-N-3-pyridylsulfonylamino)pyrimidin-4-yl]-L-4-(N,N-dimethylcarbamyloxy)phenylalanine tert-butyl ester,
N-(5-(N-methyl-N-(1-butylpyrazol-4-yl)sulfonylamino)pyrimidin-4-yl)-L-4-(N,N-dimethylcarbamyloxy)phenylalanine,
N-(5-(2,4-dimethoxypyrimidin-5-yl)pyrimidin-4-yl)-L-4-(N,N-dimethylcarbamyloxy)phenylalanine,
N-(5-(2,6-difluorophenyl)pyrimidin-4-yl)-L-4-(N,N-dimethylcarbamyloxy)phenylalanine,
N-(5-(2-hydroxymethylphenyl)pyrimidin-4-yl)-L-4-(N,N-dimethylcarbamyloxy)phenylalanine,
N-(2-(N-cyclohexylamino)-5-(2-tolyl)pyrimidin-4-yl)-L-4-(N,N-dimethylcarbamyloxy)phenylalanine,
N-(2-(N-methyl-N-(1-methylpiperidin-4-yl)amino)-5-(2-tolyl)pyrimidin-4-yl)-L-4-(N,N-dimethylcarbamyloxy)phenylalanine,
N-(2-(N-ethyl-N-isopropylamino)-5-(2-tolyl)pyrimidin-4-yl)-L-4-(N,N-dimethylcarbamyloxy)phenylalanine,
N-(5-(2,4-6-trimethylphenyl)pyrimidin-4-yl)-L-4-(N,N-dimethylcarbamyloxy)phenylalanine,
N-(5-isopropylpyrimidin-4-yl)-L-4-(N,N-dimethylcarbamyloxy)phenylalanine,
N-(2-(N-methyl-N-butylamino)-5-(2-tolyl)pyrimidin-4-yl)-L-4-(N,N-dimethylcarbamyloxy)phenylalanine,
N-(2-(N-ethyl-N-propylamino)-5-(2-tolyl)pyrimidin-4-yl)-L-4-(N,N-dimethylcarbamyloxy)phenylalanine,
N-(2-(N,N-diethylamino)-5-(2-tolyl)pyrimidin-4-yl)-L-4-(N,N-dimethylcarbamyloxy)phenylalanine,
N-(2-(N-methyl-N-ethylamino)-5-(2-tolyl)pyrimidin-4-yl)-L-4-(N,N-dimethylcarbamyloxy)phenylalanine,
N-(5-benzyloxypyrimidin-4-yl)-L-phenylalanine,
N-(5-benzyloxypyrimidin-4-yl)-L-4-(N,N-dimethylcarbamyloxy)phenylalanine,
N-(5-(N-methyl-N-4-toluenesulfonylamino)pyrimidin-4-yl)-L-phenylalanine,
N-(5-(N-methyl-N-3-pyridinesulfonylamino)pyrimidin-4-yl)-L-4-(N,N-dimethylcarbamyloxy)phenylalanine,
N-(5-phenylpyrimidin-4-yl)-L-4-(N,N-dimethylcarbamyloxy)phenylalanine,
N-(3-(N-methyl-N-4-toluenesulfonylamino)pyrazin-2-yl)-L-4-(N,N-dimethylcarbamyloxy)phenylalanine,
N-(5-(2,2,2-trifluoroethyl)pyrimidin-4-yl)-L-4-(N,N-dimethylcarbamyloxy)phenylalanine,
N-(5-(N-methyl-N-3-pyridinesulfonylamino)pyrimidin-4-yl)-L-4-(4-methylpiperazin-1-ylcarbonyloxy)phenylalanine isopropyl ester,
N-(5-benzylpyrimidin-4-yl)-L-4-(N,N-dimethylcarbamyloxy)phenylalanine,
N-(5-(N-methyl-N-3-pyridinesulfonylamino)pyrimidin-4-yl)-L-4-(4-methylpiperazin-1-ylcarbonyloxy)phenylalanine tert-butyl ester,
N-(5-(2-trifluoromethylphenyl)pyrimidin-4-yl)-L-4-(N,N-dimethylcarbamyloxy)phenylalanine,
N-(5-(2-N,N-dimethylcarbamylethyl)pyrimidin-4-yl)-L-4-(N,N-dimethylcarbamyloxy)phenylalanine,
N-(5-(N-methyl-N-3-(1-methylpyrazole)sulfonylamino)pyrimidin-4-yl)-L-4-(N,N-dimethylcarbamyloxy)phenylalanine isopropyl ester,
N-(6-phenylpyrimidin-4-yl)-L-4-(N,N-dimethylcarbamyloxy)phenylalanine,
N-(6-(2-trifluoromethylphenyl)pyrimidin-4-yl)-L-4-(N,N-dimethylcarbamyloxy)phenylalanine,
N-(6-(2-hydroxymethylphenyl)pyrimidin-4-yl)-L-4-(N,N-dimethylcarbamyloxy)phenylalanine,
N-(5-cyclohexylpyrimidin-4-yl)-L-4-(N,N-dimethylcarbamyloxy)phenylalanine,
N-(2-(N-methyl-N-2-furanmethylamino)-5-(2-tolyl)pyrimidin-4-yl)-L-4-(N,N-dimethylcarbamyloxy)phenylalanine,
N-(2-(N-methyl-N-4-chlorophenylamino)-5-(2-tolyl)pyrimidin-4-yl)-L-4-(N,N-dimethylcarbamyloxy)phenylalanine,
N-(5-(3-thienyl)pyrimidin-4-yl)-L-4-(N,N-dimethylcarbamyloxy)phenylalanine,
N-(5-(2-thienyl)pyrimidin-4-yl)-L-4-(N,N-dimethylcarbamyloxy)phenylalanine,
N-(2-(N-methyl-N-2-hydroxyethylamino)-5-(2-fluorophenyl)pyrimidin-4-yl)-L-4-(N,N-dimethylcarbamyloxy)phenylalanine,
N-(5-(piperidin-1-yl)pyrimidin-4-yl)-L-4-(N,N-dimethylcarbamyloxy)phenylalanine,
N-(5-(1-propylbutyl)pyrimidin-4-yl)-L-4-(N,N-dimethylcarbamyloxy)phenylalanine,
N-(2-(N-methyl-N-cyclobutylamino)-5-(2-tolyl)pyrimidin-4-yl)-L-4-(N,N-dimethylcarbamyloxy)phenylalanine,
N-(2-(N,N-bis-(2-hydroxyethyl)amino)pyrimidin-4-yl)-L-4-(N,N-dimethylcarbamyloxy)phenylalanine,
N-(2-(N,N-bis-(2-hydroxyethyl)amino)-5-(2-tolyl)pyrimidin-4-yl)-L-4-(N,N-dimethylcarbamyloxy)phenylalanine,
N-(2-(N-methyl-N-phenylamino)-5-(2-tolyl)pyrimidin-4-yl)-L-4-(N,N-dimethylcarbamyloxy)phenylalanine,
N-(2-(isopropoxy)-5-(2-tolyl)pyrimidin-4-yl)-L-4-(N,N-dimethylcarbamyloxy)phenylalanine,
N-(2-(N-methyl-N-3-methylbutylamino)-5-(2-tolyl)pyrimidin-4-yl)-L-4-(N,N-dimethylcarbamyloxy)phenylalanine,
N-(2-(N-methyl amino)-5-(2-tolyl)pyrimidin-4-yl)-L-4-(N,N-dimethylcarbamyloxy)phenylalanine,
N-(2-(2-tolyl)-5-(2-tolyl)pyrimidin-4-yl)-L-4-(N,N-dimethylcarbamyloxy)phenylalanine,
N-(2-(N-methyl-N-2-hydroxyethylamino)-5-(2-tolyl)pyrimidin-4-yl)-L-4-(N,N-dimethylcarbamyloxy)phenylalanine,
N-(2-(N-methyl-N-2-methylpropylamino)-5-(2-tolyl)pyrimidin-4-yl)-L-4-(N,N-dimethylcarbamyloxy)phenylalanine,
N-(2-(N-methyl-N-propylamino)-5-(2-tolyl)pyrimidin-4-yl)-L-4-(N,N-dimethylcarbamyloxy)phenylalanine,
N-(2-(N,N-dimethylamino)-5-(2-tolyl)pyrimidin-4-yl)-L-4-(N,N-dimethylcarbamyloxy)phenylalanine,
N-(2-(N-methyl-N-cyclohexylamino)-5-(3-pyridyl)pyrimidin-4-yl)-L-4-(N,N-dimethylcarbamyloxy)phenylalanine,
N-(5-(2-phenyl-2,2-difluoroethyl)pyrimidin-4-yl)-L-4-(N,N-dimethylcarbamyloxy)phenylalanine,
N-(5-(2-phenyl-2,2-difluoroethyl)-6-chloropyrimidin-4-yl)-L-4-(N,N-dimethylcarbamyloxy)phenylalanine,
N-(5-(2-phenylethyl)pyrimidin-4-yl)-L-4-(N,N-dimethylcarbamyloxy)phenylalanine,
N-(2-(N-methyl-N-cyclohexylamino)pyrimidin-4-yl)-L-4-(N,N-dimethylcarbamyloxy)phenylalanine,
N-(5-propylpyrimidin-4-yl)-L-4-(N,N-dimethylcarbamyloxy)phenylalanine,
N-(5-(2-methoxyphenyl)pyrimidin-4-yl)-L-4-(N,N-dimethylcarbamyloxy)phenylalanine,
N-(5-(2-fluorophenyl)pyrimidin-4-yl)-L-4-(N,N-dimethylcarbamyloxy)phenylalanine,
N-(2-(N-Methyl-N-isopropylamino)-5-(2-tolyl)pyrimidin-4-yl)-L-4-(N,N-dimethylcarbamyloxy)phenylalanine,
N-(2-(N-isopropylamino)-5-(2-tolyl)pyrimidin-4-yl)-L-4-(N,N-dimethylcarbamyloxy)phenylalanine,
N-(5-(2-phenylethyl)pyrimidin-4-yl)-L-4-(N,N-dimethylcarbamyloxy)phenylalanine isopropyl ester,
N-(3-(N-methyl-N-4-toluenesulfonylamino)pyrazin-2-yl)-L-phenylalanine isopropyl ester,
N-(5-(2-phenylethyl)pyrimidin-4-yl)-L-phenylalanine isopropyl ester,
N-(5-(N-methyl-N-3-pyridinesulfonylamino)pyrimidin-4-yl)-L-4-(4-methylpiperazin-1-ylcarbonyloxy)phenylalanine,
N-(2-(N-methyl-N-cyclohexylamino)-5-(2-tolyl)pyrimidin-4-yl)-L-4-(N,N-dimethylcarbamyloxy)phenylalanine,
N-(2-(N-methyl-N-cyclohexylamino)-5-ethylpyrimidin-4-yl)-L-4-(N,N-dimethylcarbamyloxy)phenylalanine,
N-(5-(2-tolyl)pyrimidin-4-yl)-L-4-(N,N-dimethylcarbamyloxy)phenylalanine isopropyl ester,
N-(5-(3-nitrophenyl)pyrimidin-4-yl)-L-4-(N,N-dimethylcarbamyloxy)phenylalanine,
N-(5-(3-pyridyl)pyrimidin-4-yl)-L-4-(N,N-dimethylcarbamyloxy)phenylalanine,
N-(5-(2-phenylethyl)pyrimidin-4-yl)-L-4-(N,N-dimethylcarbamyloxy)phenylalanine,
N-(2-N,N-dimethylamino-5-(N-methyl-N-4-toluenesulfonylamino)pyrimidin-4-yl)-L-phenylalanine,
N-(5-(2-tolyl)pyrimidin-4-yl)-L-4-(N,N-dimethylcarbamyloxy)phenylalanine,
N-(2-(N-methyl-N-cyclohexylamino)-5-(2-methoxyphenyl)pyrimidin-4-yl)-L-4-(2,6-dimethoxyphenyl)phenylalamine,
N-(2-(N-methyl-N-isopropylamino)-5-(2-fluorophenyl)pyrimidin-4-yl)-L-4-(2,6-dimethoxyphenyl)phenylalanine,
N-(2-(N-methyl-N-isopropylamino)-5-(2-fluorophenyl)pyrimidin-4-yl)-L-4-(2-methoxyphenyl)phenylalanine,
N-(2-(N-methyl-N-cyclohexylamino)-5-(2,6-difluorophenyl)pyrimidin-4-yl)-L-4-(2,6-difluorophenyl)phenylalanine,
N-(2-(N-methyl-N-cyclohexylamino)-5-(2-hydroxymethylphenyl)pyrimidin-4-yl)-L-4-(2,6-dimethoxyphenyl)phenylalanine,
N-(2-(N,N-bis-(2-hydroxyethyl)amino)-5-(2,4,6-trimethylphenyl)pyrimidin-4-yl)-L-4-(2,6-dimethoxyphenyl)phenylalanine,
N-(2-(N-methyl-N-cyclohexylamino)-5-(2-trifluoromethylphenyl)pyrimidin-4-yl)-L-4-(2-cyanophenyl)phenylalanine,
N-(2-(N-methyl-N-cyclohexylamino)-5-(3-thienyl)pyrimidin-4-yl)-L-4-(2,6-dimethoxyphenyl)phenylalanine,
N-(2-(N-methyl-N-cyclohexylamino)-5-(2-thienyl)pyrimidin-4-yl)-L-4-(4-trifluoromethylphenyl)phenylalanine,
N-(2-(N-methyl-N-cyclohexylamino)-5-(3-pyridyl)pyrimidin-4-yl)-L-4-(2,6-dimethoxyphenyl)phenylalanine,
N-(2-(N-methyl-N-cyclohexylamino)-5-(3-nitrophenyl)pyrimidin-4-yl)-L-4-(2,6-dimethoxyphenyl)phenylalanine,
N-(2-(N-methyl-N-cyclohexylamino)-5-(2,6-dichlorophenyl)pyrimidin-4-yl)-L-4-(2,6-dimethoxyphenyl)phenylalanine,
N-(2-(N-methyl-N-cyclohexylamino)-5-(4-pyridyl)pyrimidin-4-yl)-L-4-(3-hydroxymethylphenyl)phenylalanine,
N-(2-(N-ethyl-N-isopropylamino)-5-(2,6-dimethoxyphenyl)pyrimidin-4-yl)-L-4-(2,6-dimethoxyphenyl)phenylalanine,
N-(2-(N-methyl-N-cyclohexylamino)-5-(2,3-dichlorophenyl)pyrimidin-4-yl)-L-4-(2,6-dimethoxyphenyl)phenylalanine,
N-(2-(N-methyl-N-ethylamino)-5-(2,4,6-trimethylphenyl)pyrimidin-4-yl)-L-4-(2-cyanophenyl)phenylalanine,
N-(2-(N-methyl-N-isopropylamino)-5-(2,4,6-trimethylphenyl)pyrimidin-4-yl)-L-4-(3-pyridyl)phenylalanine,
N-(2-(N,N-bis-(2-hydroxyethyl)amino)-5-(2,4,6-trimethylphenyl)pyrimidin-4-yl)-L-4-(2-cyanophenyl)phenylalanine,
N-(2-(N-methyl-N-(1-methylpiperidin-4-yl)amino)-5-(2-cyanophenyl)pyrimidin-4-yl)-L-4-(2,6-difluorophenyl)phenylalanine,
N-(2-(N-ethyl-N-isopropylamino)-5-(2,4,6-trimethylphenyl)pyrimidin-4-yl)-L-4-(o-tolyl)phenylalanine,
N-(2-(N-methyl-N-4-chlorophenylamino)-5-(2,4,6-trimethylphenyl)pyrimidin-4-yl)-L-4-(2,6-dimethoxyphenyl)phenylalanine,
N-(5-(N-methyl-N-2-(phenyl)ethylamino)pyrimidin-4-yl)-L-4-(N,N-dimethylcarbamyloxy)phenylalanine,
N-(5-(N-methyl-N-hexylamino)pyrimidin-4-yl)-L-4-(N,N-dimethylcarbamyloxy)phenylalanine,
N-(5-(N-methyl-N-isopropylamino)pyrimidin-4-yl)-L-4-(N,N-dimethylcarbamyloxy)phenylalanine,
N-(5-(N-methyl-N-tert-butylamino)pyrimidin-4-yl)-L-4-(N,N-dimethylcarbamyloxy)phenylalanine,
N-(5-(N-ethyl-N-isopropylamino)pyrimidin-4-yl)-L-4-(N,N-dimethylcarbamyloxy)phenylalanine,
N-(5-(N-methyl-N-2-(4-pyridyl)ethyl-pyrimidin-4-yl)-L-4-(N,N-dimethylcarbamyloxy)phenylalanine,
N-(5-(N-methyl-N-2-(phenyl)ethylamino)pyrimidin-4-yl)-L-4-(4-(2,6-dimethoxyphenyl)phenylalanine,
N-(5-(N-methyl-N-hexylamino)pyrimidin-4-yl)-L-4-(2,6-dimethoxyphenyl)phenylalanine,
N-(5-(N-methyl-N-isopropylamino)pyrimidin-4-yl)-L-4-(2,6-dimethoxyphenyl)phenylalanine,
N-(5-(N-methyl-N-tert-butylamino)pyrimidin-4-yl)-L-4-(2,6-dimethoxyphenyl)phenylalanine,
N-(5-(N-ethyl-N-isopropylamino)pyrimidin-4-yl)-L-4-(2,6-dimethoxyphenyl)phenylalanine,
N-(5-(N-methyl-N-2-(4-pyridyl)ethyl-pyrimidin-4-yl)-L-4-(2,6-dimethoxyphenyl)phenylalanine,
N-(2-(N-methyl-N-cyclohexylamino)-5-ethylpyrimidin-4-yl)-L-4-(N,N-dimethylcarbamyloxy)phenylalanine,
N-(4-(N,N-di-n-hexylamino)-1,1-dioxo-1,2,5-thiadiazol-3-yl)-L-tyrosine,
N-(4-(N,N-di-n-hexylamino)-1,1-dioxo-1,2,5-thiadiazol-3-yl)-L-4-(N,N-dimethylcarbamyloxy)phenylalanine,
N-(4-(N,N-dimethylamino)-1-oxo-1,2,5-thiadiazol-3-yl)-L-4-(N,N-dimethylcarbamyloxy)phenylalanine tert-butyl ester,
N-[4-(2-(3-methylphenylaminocarbonylamino)eth-1-ylamino)-1,1-dioxo-1,2,5-thiadiazol-3-yl]-L-4-(N,N-dimethylcarbamyloxy)phenylalanine
N-(4-(N,N-di-n-hexylamino)-1,1-dioxo-1,2,5-thiadiazol-3-yl)-L-4-(4-methylpiperazin-1-ylcarbonyloxy)phenylalanine,
N-(5-(2,2,2-trifluoroethyl)pyrimidin-4-yl)-L-4-(2,6-dimethoxyphenyl)phenylalanine,
N-(2-(N-cyclohexyl-N-methyl)-5-(2-tolyl)pyrimidin-4-yl)-L-4-(2,6-dimethoxyphenyl)phenylalanine,
N-(5-(2-fluorophenyl)pyrimidin-4-yl)-L-4-(2,6-dimethoxyphenyl)phenylalanine,
N-(2-(N-methyl-N-propyl)-5-(2-tolyl)pyrimidin-4-yl)-L-4-(2,6-dimethoxyphenyl)phenylalanine,
N-(3-chloropyrazin-2-yl)-L-4-[1-(tert-butoxycarbonyl)piperidin-4-ylcarbonylamino]phenylalanine ethyl ester,
   and pharmaceutically acceptable salts thereof.

Further description of the compounds of the above formulae IIIa, IIIb, VIa, VIb, VIc, VId, Va, Vb, Vc, Vd, VIa, VIb, VIc, and VId procedures and reaction conditions for preparing these compounds are described in U.S. Ser. No. 09/489,377 (filed Jan. 21, 2000, and issued as U.S. Pat. No. 6,492,372), herein incorporated by reference in its entirety.

Further description of the compounds of the above formulae IIIa, IIIb, VIa, VIb, VIc, VId, Va, Vb, Vc, Vd, VIa, VIb, VIc, and VId procedures and reaction conditions for preparing these compounds are also described in U.S. Patent Publication 2003/0139402, a divisional application of U.S. Ser. No. 09/489,377, herein incorporated by reference in its entirety.

Definitions

When describing the compounds of formulae IIIa, IIIb, VIa, VIb, VIc, VId, Va, Vb, Vc, Vd, VIa, VIb, VIc, and VId, compositions comprising compound of formulae IIIa, IIIb, VIa, VIb, VIc, VId, Va, Vb, Vc, Vd, VIa, VIb, VIc, and VId, and methods of this invention for compounds of formulae IIIa, IIIb, VIa, VIb, VIc, VId, Va, Vb, Vc, Vd, VIa, VIb, VIc, and VId, the following terms have the following meanings, unless otherwise indicated.

As used herein, “alkyl” refers to alkyl groups preferably having from 1 to 10 carbon atoms and more preferably 1 to 6 carbon atoms. This term is exemplified by groups such as methyl, t-butyl, n-heptyl, octyl and the like.

“Substituted alkyl” refers to an alkyl group, preferably of from 1 to 10 carbon atoms, having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Alkenoxy” refers to the group “alkenyl-O—”.

“Substituted alkenoxy” refers to the group “substituted alkenyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)-cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocarbonylamino” refers to the group —C(S)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form, together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxysulfonyl” refers to the groups alkyl-SO₂O—, substituted alkyl-SO₂O—, alkenyl-SO₂O—, substituted alkenyl-SO₂O—, alkynyl-SO₂O—, substituted alkynyl-SO₂O—, aryl-SO₂O—, substituted aryl-SO₂O—, cycloalkyl-SO₂O—, substituted cycloalkyl-SO₂O—, heteroaryl-SO₂O—, substituted heteroaryl-SO₂O—, heterocyclic-SO₂O—, and substituted heterocyclic-SO₂O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 10 carbon atoms and more preferably 2 to 6 carbon atoms and having at least 1 and preferably from 1-2 sites of alkenyl unsaturation.

“Substituted alkenyl” refers to alkenyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynyl” refers to alkynyl group preferably having from 2 to 10 carbon atoms and more preferably 3 to 6 carbon atoms and having at least 1 and preferably from 1-2 sites of alkynyl unsaturation.

“Substituted alkynyl” refers to alkynyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Amidino” refers to the group H₂NC(═NH)— and the term “alkylamidino” refers to compounds having 1 to 3 alkyl groups (e.g., alkylHNC(═NH)—).

“Thioamidino” refers to the group RSC(═NH)— where R is hydrogen or alkyl.

“Amino” refers to the group —NH₂.

“Substituted amino” refers to the group —NRR, where each R group is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, provided that both R groups are not hydrogen; or the R groups can be joined together with the nitrogen atom to form a heterocyclic or substituted heterocyclic ring.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the groups —NRSO₂alkyl, —NRSO₂substituted alkyl, —NRSO₂cycloalkyl, —NRSO₂substituted cycloalkyl, —NRSO₂alkenyl, —NRSO₂substituted alkenyl, —NRSO₂alkynyl, —NRSO₂substituted alkynyl, —NRSO₂aryl, —NRSO₂substituted aryl, —NRSO₂heteroaryl, —NRSO₂substituted heteroaryl, —NRSO₂heterocyclic, and —NRSO₂substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the groups —NRC(O)O-alkyl, —NRC(O)O-substituted alkyl, —NRC(O)O-alkenyl, —NRC(O)O-substituted alkenyl, —NRC(O)O-alkynyl, —NRC(O)O-substituted alkynyl, —NRC(O)O-cycloalkyl, —NRC(O)O-substituted cycloalkyl, —NRC(O)O-aryl, —NRC(O)O-substituted aryl, —NRC(O)O-heteroaryl, —NRC(O)O-substituted heteroaryl, —NRC(O)O-heterocyclic, and —NRC(O)O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the groups —NRSO₂₀-alkyl, —NRSO₂₀-substituted alkyl, —NRSO₂₀-alkenyl, —NRSO₂₀-substituted alkenyl, —NRSO₂₀-alkynyl, —NRSO₂O-substituted alkynyl, —NRSO₂O-cycloalkyl, —NRSO₂O-substituted cycloalkyl, —NRSO₂O-aryl, —NRSO₂O-substituted aryl, —NRSO₂O-heteroaryl, —NRSO₂O-substituted heteroaryl, —NRSO₂O-heterocyclic, and —NRSO₂O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxycarbonylamino” refers to the groups —OC(O)NH₂, —OC(O)NRR, —OC(O)NR-alkyl, —OC(O)NR-substituted alkyl, —OC(O)NR-alkenyl, —OC(O)NR-substituted alkenyl, —OC(O)NR-alkynyl, —OC(O)NR-substituted alkynyl, —OC(O)NR-cycloalkyl, —OC(O)NR-substituted cycloalkyl, —OC(O)NR-aryl, —OC(O)NR-substituted aryl, —OC(O)NR-heteroaryl, —OC(O)NR-substituted heteroaryl, —OC(O)NR-heterocyclic, and —OC(O)NR-substituted heterocyclic where R is hydrogen, alkyl or where each R is joined to form, together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NH₂, —OC(S)NRR, —OC(S)NR-alkyl, —OC(S)NR-substituted alkyl, —OC(S)NR-alkenyl, —OC(S)NR-substituted alkenyl, —OC(S)NR-alkynyl, —OC(S)NR-substituted alkynyl, —OC(S)NR-cycloalkyl, —OC(S)NR-substituted cycloalkyl, —OC(S)NR-aryl, —OC(S)NR-substituted aryl, —OC(S)NR-heteroaryl, —OC(S)NR-substituted heteroaryl, —OC(S)NR-heterocyclic, and —OC(S)NR-substituted heterocyclic where R is hydrogen, alkyl or where each R is joined to form together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxysulfonylamino” refers to the groups —OSO₂NH₂, —OSO₂NRR, —OSO₂NR-alkyl, —OSO₂NR-substituted alkyl, —OSO₂NR-alkenyl, —OSO₂NR-substituted alkenyl, —OSO₂NR-alkynyl, —OSO₂NR-substituted alkynyl, —OSO₂NR-cycloalkyl, —OSO₂NR-substituted cycloalkyl, —OSO₂NR-aryl, —OSO₂NR-substituted aryl, —OSO₂NR-heteroaryl, —OSO₂NR-substituted heteroaryl, —OSO₂NR-heterocyclic, and —OSO₂NR-substituted heterocyclic where R is hydrogen, alkyl or where each R is joined to form, together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the groups —NRC(O)NRR, —NRC(O)NR-alkyl, —NRC(O)NR-substituted alkyl, —NRC(O)NR-alkenyl, —NRC(O)NR-substituted alkenyl, —NRC(O)NR-alkynyl, —NRC(O)NR-substituted alkynyl, —NRC(O)NR-aryl, —NRC(O)NR-substituted aryl, —NRC(O)NR-cycloalkyl, —NRC(O)NR-substituted cycloalkyl, —NRC(O)NR-heteroaryl, and —NRC(O)NR-substituted heteroaryl, —NRC(O)NR-heterocyclic, and —NRC(O)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the groups —NRC(S)NRR, —NRC(S)NR-alkyl, —NRC(S)NR-substituted alkyl, —NRC(S)NR-alkenyl, —NRC(S)NR-substituted alkenyl, —NRC(S)NR-alkynyl, —NRC(S)NR-substituted alkynyl, —NRC(S)NR-aryl, —NRC(S)NR-substituted aryl, —NRC(S)NR-cycloalkyl, —NRC(S)NR-substituted cycloalkyl, —NRC(S)NR-heteroaryl, and —NRC(S)NR-substituted heteroaryl, —NRC(S)NR-heterocyclic, and —NRC(S)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the groups —NRSO₂NRR, —NRSO₂NR-alkyl, —NRSO₂NR-substituted alkyl, —NRSO₂NR-alkenyl, —NRSO₂NR-substituted alkenyl, —NRSO₂NR-alkynyl, —NRSO₂NR-substituted alkynyl, —NRSO₂NR-aryl, —NRSO₂NR-substituted aryl, —NRSO₂NR-cycloalkyl, —NRSO₂NR-substituted cycloalkyl, —NRSO₂NR-heteroaryl, and —NRSO₂NR-substituted heteroaryl, —NRSO₂NR-heterocyclic, and —NRSO₂NR-substituted heterocyclic, where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7yl, and the like). Preferred aryls include phenyl and naphthyl.

Substituted aryl refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Cycloalkyl” refers to cyclic alkyl groups of from 3 to 8 carbon atoms having a single cyclic ring including, by way of example, cyclopropyl, cyclobutyl, cyclopentyl, cyclohexyl, cyclooctyl and the like. Excluded from this definition are multi-ring alkyl groups such as adamantanyl, etc.

“Cycloalkenyl” refers to cyclic alkenyl groups of from 3 to 8 carbon atoms having single or multiple unsaturation but which are not aromatic.

“Substituted cycloalkyl” and “substituted cycloalkenyl” refer to a cycloalkyl and cycloalkenyl groups, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkoxy” refers to —O-cycloalkyl groups.

“Substituted cycloalkoxy” refers to —O-substituted cycloalkyl groups.

“Cycloalkenoxy” refers to —O-cycloalkenyl groups.

“Substituted cycloalkenoxy” refers to —O-substituted cycloalkenyl groups.

“Guanidino” refers to the groups —NRC(═NR)NRR, —NRC(═NR)NR-alkyl, —NRC(═NR)NR-substituted alkyl, —NRC(═NR)NR-alkenyl, —NRC(═NR)NR-substituted alkenyl, —NRC(═NR)NR-alkynyl, —NRC(═NR)NR-substituted alkynyl, —NRC(═NR)NR-aryl, —NRC(═NR)NR-substituted aryl, —NRC(═NR)NR-cycloalkyl, —NRC(═NR)NR-heteroaryl, —NRC(═NR)NR-substituted heteroaryl, —NRC(═NR)NR-heterocyclic, and —NRC(═NR)NR-substituted heterocyclic where each R is independently hydrogen and alkyl as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Guanidinosulfone” refers to the groups —NRC(═NR)NRSO₂-alkyl, —NRC(═NR)NRSO₂-substituted alkyl, —NRC(═NR)NRSO₂-alkenyl, —NRC(═NR)NRSO₂-substituted alkenyl, —NRC(═NR)NRSO₂-alkynyl, —NRC(═NR)NRSO₂-substituted alkynyl, —NRC(═NR)NRSO₂-aryl, —NRC(═NR)NRSO₂-substituted aryl, —NRC(═NR)NRSO₂-cycloalkyl, —NRC(═NR)NRSO₂-substituted cycloalkyl, —NRC(═NR)NRSO₂-heteroaryl, and —NRC(═NR)NRSO₂-substituted heteroaryl, —NRC(═NR)NRSO₂-heterocyclic, and —NRC(═NR)NRSO₂-substituted heterocyclic where each R is independently hydrogen and alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Halo” or “halogen” refers to fluoro, chloro, bromo and iodo and preferably is either chloro or bromo.

“Heteroaryl” refers to an aromatic carbocyclic group of from 2 to 10 carbon atoms and 1 to 4 heteroatoms selected from oxygen, nitrogen and sulfur within the ring or oxides thereof. Such heteroaryl groups can have a single ring (e.g., pyridyl or furyl) or multiple condensed rings (e.g., indolizinyl or benzothienyl). Additionally, the heteroatoms of the heteroaryl group may be oxidized, i.e., to form pyridine N-oxides or 1,1-dioxo-1,2,5-thiadiazoles and the like. Preferred heteroaryls include pyridyl, pyrrolyl, indolyl, furyl, pyridazinyl, pyrimidinyl, pyrazinyl, 1-oxo-1,2,5-thiadiazolyl and 1,1-dioxo-1,2,5-thiadiazolyl. The term “heteroaryl having two nitrogen atoms in the heteroaryl ring” refers to a heteroaryl group having two, and only two, nitrogen atoms in the heteroaryl ring and optionally containing 1 or 2 other heteroatoms in the heteroaryl ring, such as oxygen or sulfur “Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from nitrogen, sulfur or oxygen within the ring wherein, in fused ring systems, one or more of the rings can be aryl or heteroaryl.

“Substituted heterocyclic” refers to heterocycle groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“Thiol” refers to the group —SH.

“Thioalkyl” refers to the groups —S-alkyl “Substituted thioalkyl” refers to the group —S-substituted alkyl.

“Thiocycloalkyl” refers to the groups —S-cycloalkyl.

“Substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

“Optionally subsituted” means that the recited group may be unsubstituted or the recited group may be substituted.

Compound Preparation for Compounds of Formulae IIIa, IIIb, IVa, IVb, IVc, IVd, Va, Vb, Vc Vd, VIa, VIb, VIc, and VId

The compounds of formulae IIIa, IIIb, IVa, IVb, IVc, IVd, Va, Vb, Vc, Vd, VIa, VIb, VIc, and VId can be prepared from readily available starting materials using the following general methods and procedures as described in U.S. Pat. No. 6,492,372 and Patent Publication 2003/0139402. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Compounds of Formulae VII-XX

In another aspect, the compounds that can be utilized as steroid sparing agents for treatment of a subject, with a disease selected from the group consisting of multiple sclerosis, asthma, rheumatoid arthritis, graft versus host disease, host versus graft disease, and spondyloarthropathies, are compounds of formulae VII-XX.

In one aspect, the compounds that can be utilized as steroid sparing agents are compounds defined by formula VII below. These compounds have a binding affinity to VLA-4 as expressed by an IC₅₀ of about 15 μM or less (measured as described in Example A below): 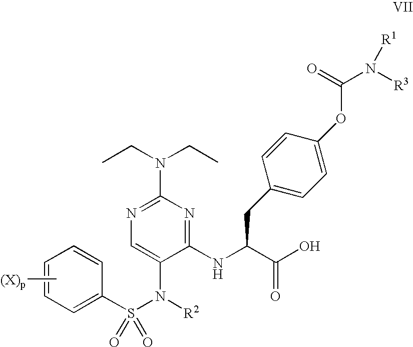
wherein each X is independently fluoro, chloro or bromo;
p is an integer from 0 to 3;
R¹ and R³ together with the nitrogen atom to which they are bound form an azetidinyl, pyrrolidinyl, pyrrolyl, 2,5-dihydopyrrol-1-yl, piperidinyl, or 1,2,3,6-tetrahydropyridin-1-yl;
R² is selected from the group consisting of lower alkyl, lower alkenyl, and lower alkylenecycloalkyl;
and pharmaceutically acceptable salts thereof.

In a preferred embodiment, R¹ and R³ together with the nitrogen atom to which they are bound form an azetidinyl, pyrrolidinyl, or piperidinyl group.

In one aspect, the compounds that can be utilized as steroid sparing agents are compounds defined by formula VIII below. These compounds have a binding affinity to VLA-4 as expressed by an IC₅₀ of about 15 μM or less (measured as described in Example A below): 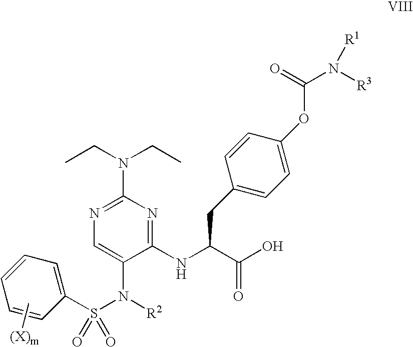
wherein each X is independently selected from the group consisting of fluoro and chloro;
m is an integer equal to 1 or 2;
R² is selected from the group consisting of lower alkyl, lower alkenyl, and lower alkylenecycloalkyl;
R¹ and R³ together with the nitrogen atom to which they are bound form an azetidinyl, pyrrolidinyl, or piperidinyl group;
and pharmaceutically acceptable salts thereof.

In one aspect, the compounds that can be utilized as steroid sparing agents are compounds defined by formula IX below. These compounds have a binding affinity to VLA-4 as expressed by an IC₅₀ of about 15 [M or less (measured as described in Example A below): 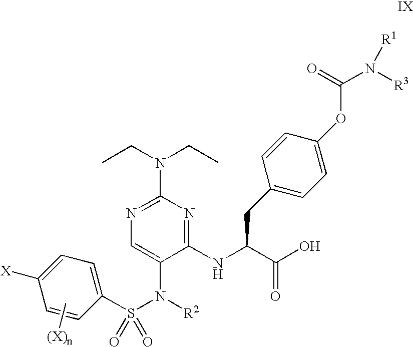
wherein each X is independently fluoro or chloro;
n is zero or one;
R² is —CH₂—R′ where R′ is selected from the group consisting of hydrogen, methyl or —CH═CH₂;
R¹ and R³ together with the nitrogen atom to which they are bound form an azetidinyl, pyrrolidinyl, or piperidinyl group;
and pharmaceutically acceptable salts thereof.

In one aspect, the compounds that can be utilized as steroid sparing agents are compounds defined by formula X below. These compounds have a binding affinity to VLA-4 as expressed by an IC₅₀ of about 15 μM or less (measured as described in Example A below): 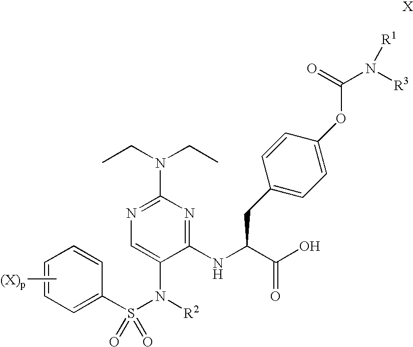
wherein each X is independently fluoro, chloro or bromo;
p is an integer from 0 to 3;
R¹ and R³ together with the nitrogen atom to which they are bound form an azetidinyl, pyrrolidinyl, pyrrolyl, 2,5-dihydopyrrol-1-yl, piperidinyl, or 1,2,3,6-tetrahydropyridin-1-yl;
R² is lower alkynyl;
and pharmaceutically acceptable salts thereof.

In a preferred embodiment, R¹ and R³ together with the nitrogen atom to which they are bound form an azetidinyl, pyrrolidinyl, or piperidinyl group and R² is propargyl.

In another aspect, the compounds that can be utilized as steroid sparing agents are compounds defined by formula XI below. These compounds have a binding affinity to VLA-4 as expressed by an IC₅₀ of about 15 μM or less (measured as described in Example A below): 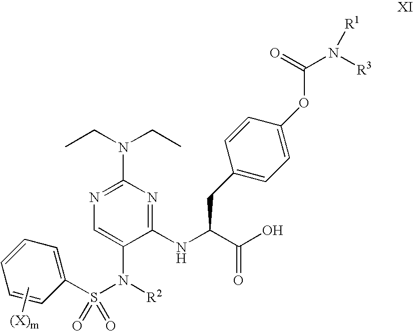
wherein each X is independently selected from the group consisting of fluoro and chloro;
m is an integer equal to 1 or 2;
R² is lower alkynyl;
R¹ and R³ together with the nitrogen atom to which they are bound form an azetidinyl, pyrrolidinyl, or piperidinyl group;
and pharmaceutically acceptable salts thereof.

In one aspect, the compounds that can be utilized as steroid sparing agents are compounds defined by formula XII below. These compounds have a binding affinity to VLA-4 as expressed by an IC₅₀ of about 15 μM or less (measured as described in Example A below): 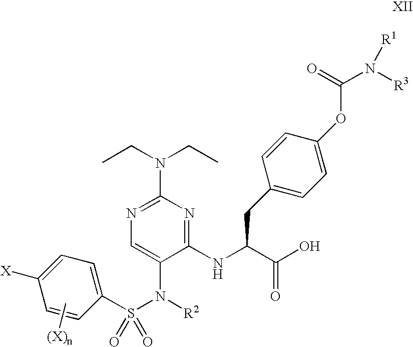
wherein each X is independently fluoro or chloro;
n is zero or one;
R² is lower alkynyl;
R¹ and R³ together with the nitrogen atom to which they are bound form an azetidinyl, pyrrolidinyl, or piperidinyl group;
and pharmaceutically acceptable salts thereof.

N-[2-N′,N′-diethylamino-5-aminosulfonylphenylpyrimidin-4-yl]-p-carbomyloxy-phenylalanine compounds within the scope of this invention include those set forth in Table 5 as follows:

TABLE 5

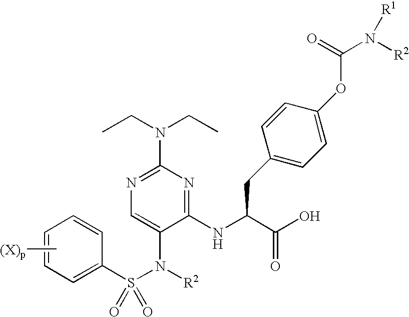

| | | | Example |
|---|---|---|---|
| R¹ and R³ | R² | 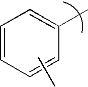 | No. |
| pyrrolidinyl | ethyl | 4-chlorophenyl | 505 |
| pyrrolidinyl | ethyl | 4-fluorophenyl | 506 |
| pyrrolidinyl | methyl | 4-fluorophenyl | 507 |
| pyrrolidinyl | methyl | 4-chlorophenyl | 508 |
| piperidinyl | methyl | 4-fluorophenyl | 509 |
| piperidinyl | ethyl | 4-fluorophenyl | 510 |
| azetidinyl | ethyl | 4-fluorophenyl | 511 |
| azetidinyl | methyl | 4-fluorophenyl | 512 |
| azetidinyl | methyl | 4-chlorophenyl | 513 |
| azetidinyl | ethyl | 4-chlorophenyl | 514 |
| pyrrolidinyl | methyl | 2,4-difluorophenyl | 515 |
| pyrrolidinyl | ethyl | 2,4-difluorophenyl | 516 |
| azetidinyl | methyl | 2,4-difluorophenyl | 517 |
| azetidinyl | ethyl | 2,4-difluorophenyl | 518 |
| pyrrolidinyl | propargyl | 4-fluorophenyl | 519 |
| pyrrolidinyl | progargyl | 2,4-difluorophenyl | 520 |
| azetidinyl | propargyl | 2,4-difluorophenyl | 521 |
| azetidinyl | propargyl | 4-fluorophenyl | 522 |
| pyrrolidinyl | progargyl | 4-chlorophenyl | 523 |

Specific compounds within the scope of this invention include the following compounds. As used below, these compounds are named based on phenylalanine derivatives but, alternatively, these compounds could have been named based on N-[2-N′,N′-diethylamino-5-aminosulfonylphenyl-pyrimidin-4-yl]-p-carbomyloxyphenylalanine derivatives or 2-{2-diethylamino-5-[(benzenesulfonyl)methylamino]-pyrimidin-4-ylamino}-p-carbamoyloxy-phenyl)propionic acid derivatives.
N-(2-[N′,N′-diethylamino]-5-[N″-(4-chlorophenylsulfonyl)-N″-ethylamino]pyrimidin-4-yl)-4′-(pyrrolidin-1-ylcarbonyloxy)-L-phenylalanine;
N-(2-[N′,N′-diethylamino]-5-[N″-(4-fluorophenylsulfonyl)-N″-ethylamino]pyrimidin-4-yl)-4′-(pyrrolidin-1-ylcarbonyloxy)-L-phenylalanine;
N-(2-[N′,N′-diethylamino]-5-[N″-(4-fluorophenylsulfonyl)-N″-methylamino]pyrimidin-4-yl)-4′-(pyrrolidin-1-ylcarbonyloxy)-L-phenylalanine;
N-(2-[N′,N′-diethylamino]-5-[N″-(4-chlorophenylsulfonyl)-N″-methylamino]pyrimidin-4-yl)-4′-(pyrrolidin-1-ylcarbonyloxy)-L-phenylalanine;
N-(2-[N′,N′-diethylamino]-5-[N″-(4-fluorophenylsulfonyl)-N″-methylamino]pyrimidin-4-yl)-4′-(piperidin-1-ylcarbonyloxy)-L-phenylalanine;
N-(2-[N′,N′-diethylamino]-5-[N″-(4-fluorophenylsulfonyl)-N″-ethylamino]pyrimidin-4-yl)-4′-(piperidin-1-ylcarbonyloxy)-L-phenylalanine;
N-(2-[N′,N′-diethylamino]-5-[N″-(4-fluorophenylsulfonyl)-N″-ethylamino]pyrimidin-4-yl)-4′-(azetidin-1-ylcarbonyloxy)-L-phenylalanine;
N-(2-[N′,N′-diethylamino]-5-[N″-(4-fluorophenylsulfonyl)-N″-methylamino]pyrimidin-4-yl)-4′-(azetidin-1-ylcarbonyloxy)-L-phenylalanine;
N-(2-[N′,N′-diethylamino]-5-[N″-(4-chlorophenylsulfonyl)-N″-methylamino]pyrimidin-4-yl)-4′-(azetidin-1-ylcarbonyloxy)-L-phenylalanine;
N-(2-[N′,N′-diethylamino]-5-[N″-(4-chlorophenylsulfonyl)-N″-ethylamino]pyrimidin-4-yl)-4′-(azetidin-1-ylcarbonyloxy)-L-phenylalanine;
N-(2-[N′,N′-diethylamino]-5-[N″-(2,4-difluorophenylsulfonyl)-N″-methylamino]pyrimidin-4-yl)-4′-(pyrrolidin-1-ylcarbonyloxy)-L-phenylalanine;
N-(2-[N′,N′-diethylamino]-5-[N″-(2,4-difluorophenylsulfonyl)-N″-ethylamino]pyrimidin-4-yl)-4′-(pyrrolidin-1-ylcarbonyloxy)-L-phenylalanine;
N-(2-[N′,N′-diethylamino]-5-[N″-(2,4-difluorophenylsulfonyl)-N″-methylamino]pyrimidin-4-yl)-4′-(azetidin-1-ylcarbonyloxy)-L-phenylalanine;
N-(2-[N′,N′-diethylamino]-5-[N″-(2,4-difluorophenylsulfonyl)-N″-ethylamino]pyrimidin-4-yl)-4′-(azetidin-1-ylcarbonyloxy)-L-phenylalanine;
N-(2-[N′,N′-diethylamino]-5-[N″-(4-fluorophenylsulfonyl)-N″-propargylamino]pyrimidin-4-yl)-4′-(pyrrolidin-1-ylcarbonyloxy)-L-phenylalanine;
N-(2-[N′,N′-diethylamino]-5-[N″-(2,4-difluorophenylsulfonyl)-N″-propargylamino]pyrimidin-4-yl)-4′-(pyrrolidin-1-ylcarbonyloxy)-L-phenylalanine;
N-(2-[N′,N′-diethylamino]-5-[N″-(2,4-difluorophenylsulfonyl)-N″-propargylamino]pyrimidin-4-yl)-4′-(azetidin-1-ylcarbonyloxy)-L-phenylalanine;
N-(2-[N′,N′-diethylamino]-5-[N″-(4-fluorophenylsulfonyl)-N″-propargylamino]pyrimidin-4-yl)-4′-(azetidin-1-ylcarbonyloxy)-L-phenylalanine;
N-(2-[N′,N′-diethylamino]-5-[N″-(4-chlorophenylsulfonyl)-N″-propargylamino]pyrimidin-4-yl)-4′-(pyrrolidin-1-ylcarbonyloxy)-L-phenylalanine; and
   pharmaceutically acceptable salts thereof.

Preferably, the compound is the compound of formula XIII below:

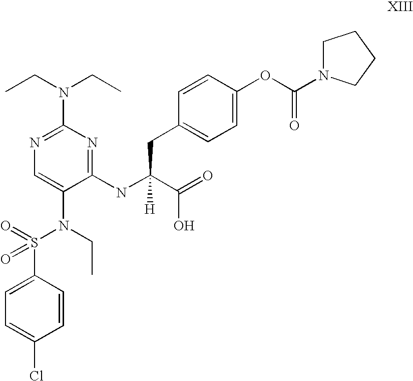

In another embodiment, preferably the compound is the compound of formula XIV below: 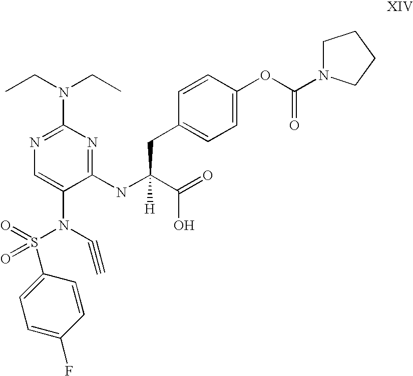
Compound Preparation of Compounds of Formulae VII-XIV

The compounds of formulae VII-XIV can be prepared from readily available starting materials using the methods and procedures set forth in the examples below. These methods and procedures outline specific reaction protocols for preparing N-[2-N′,N′-diethylamino-5-aminosulfonylphenyl-yrimidin-4-yl]-p-carbomyloxy-phenylalanine compounds. Compounds within the scope not exemplified in these examples and methods are readily prepared by appropriate substitution of starting materials which are either commercially available or well known in the art.

Other procedures and reaction conditions for preparing the compounds of this invention are described in the examples set forth below. Additionally, other procedures for preparing compounds useful in certain aspects of this invention are disclosed in U.S. Pat. No. 6,492,372, issued Dec. 10, 2002; the disclosure of which is incorporated herein by reference in its entirety.

Further description of the compounds of formulae VII-XIV, procedures, and reaction conditions for preparing these compounds are described in U.S. Patent Publication 2004/0138243, herein incorporated by reference in its entirety.

Compounds of Formulae XV-XX

In another aspect, the compounds that can be utilized as steroid sparing agents are compounds defined by formula XV below. These compounds have a binding affinity to VLA-4 as expressed by an IC₅₀ of about 15 μM or less (measured as described in Example A below): 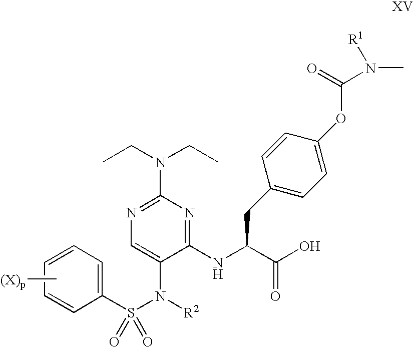
wherein each X is independently fluoro, chloro or bromo;
p is 0 or an integer from 1-3;
R¹ is selected from the group consisting of methyl and ethyl;
R² is selected from the group consisting of lower alkyl, lower alkenyl, and lower alkylenecycloalkyl;
and pharmaceutically acceptable salts thereof.

In one aspect, the compounds that can be utilized as steroid sparing agents are compounds defined by formula XVI below. These compounds have a binding affinity to VLA-4 as expressed by an IC₅₀ of about 15 μM or less (measured as described in Example A below): 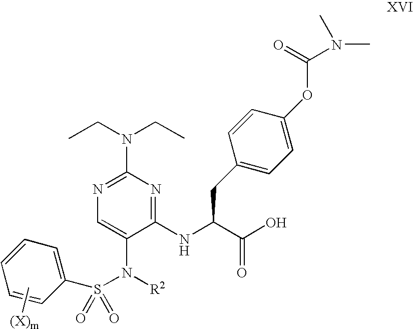
wherein each X is independently selected from the group consisting of fluoro and chloro,
m is an integer equal to 1 or 2;
R² is selected from the group consisting of lower alkyl, lower alkenyl, and lower alkylenecycloalkyl;
and pharmaceutically acceptable salts thereof.

In one aspect, the compounds that can be utilized as steroid sparing agents are compounds defined by formula XVII below. These compounds have a binding affinity to VLA-4 as expressed by an IC₅₀ of about 15 μM or less (measured as described in Example A below): 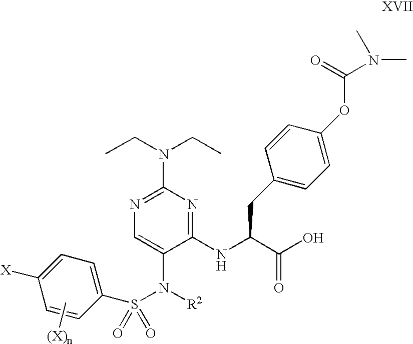
wherein each X is independently fluoro or chloro;
n is zero or one;
R² is —CH₂—R′ where R′ is selected from the group consisting of hydrogen, methyl or —CH═CH₂;
and pharmaceutically acceptable salts thereof.

In one aspect, the compounds that can be utilized as steroid sparing agents are compounds defined by formula XVIII below. These compounds have a binding affinity to VLA-4 as expressed by an IC₅₀ of about 15 μM or less (measured as described in Example A below): 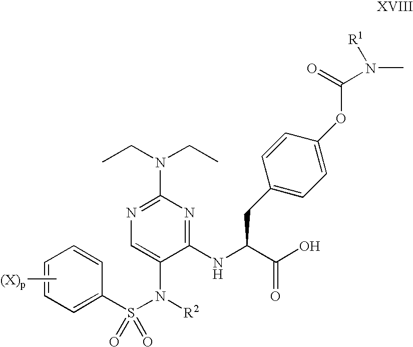
wherein each X is independently fluoro, chloro or bromo;
p is 0 or an integer from 1-3;
R¹ is selected from the group consisting of methyl and ethyl;
R² is lower alkynyl;
and pharmaceutically acceptable salts thereof.

In one aspect, the compounds that can be utilized as steroid sparing agents are compounds defined by formula XIX below. These compounds have a binding affinity to VLA-4 as expressed by an IC₅₀ of about 15 μM or less (measured as described in Example A below): 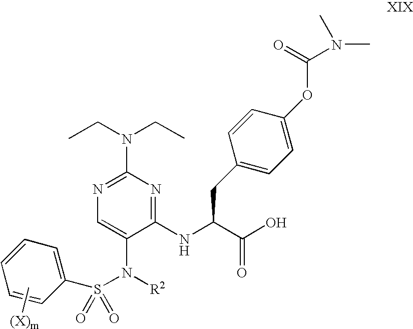
wherein each X is independently selected from the group consisting of fluoro and chloro,
m is an integer equal to 1 or 2;
R² is lower alkynyl;
and pharmaceutically acceptable salts thereof.

In one aspect, the compounds that can be utilized as steroid sparing agents are compounds defined by formula XX below. These compounds have a binding affinity to VLA-4 as expressed by an IC₅₀ of about 15 μM or less (measured as described in Example A below): 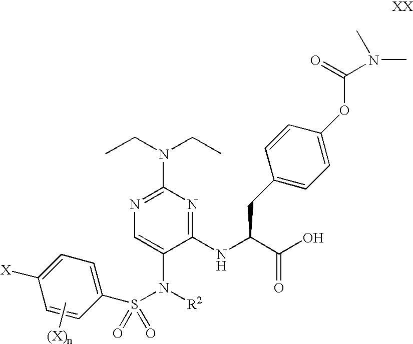
wherein each X is independently fluoro or chloro;
n is zero or one;
R² is lower alkynyl;
and pharmaceutically acceptable salts thereof.
R² is preferably propargyl in any of one of formulae XVII-XX.

N-[2-N′,N′-diethylamino-5-aminosulfonylphenylpyrimidin-4-yl]-p-carbomyloxyphenylalanine compounds within the scope of this invention include those set forth in Table 6 as follows:

TABLE 6

| | |
|---|---|
| | XVI |
| 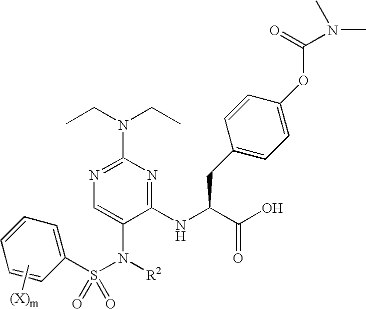 | |

| Example No. | 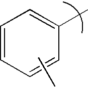 | R² |
|---|---|---|
| 524 | 4-fluorophenyl | methyl |
| 525 | 4-chlorophenyl | methyl |
| 526 | 3,4-difluorophenyl | methyl |
| 527 | 3,4-dichiorophenyl | methyl |
| 528 | phenyl | methyl |
| 529 | 2-fluorophenyl | methyl |
| 530 | 3-fluorophenyl | methyl |
| 531 | 4-fluorophenyl | isopropyl |
| 532 | 4-fluorophenyl | ethyl |
| 533 | 3,4-difluorophenyl | isopropyl |
| 534 | 4-chlorophenyl | isopropyl |
| 535 | 3,4-difluorophenyl | ethyl |
| 536 | 4-chlorophenyl | ethyl |
| 537 | 4-fluorophenyl | cyclopropylmethyl |
| 538 | 3,5-difluorophenyl | methyl |
| 539 | 3,5-difluorophenyl | ethyl |
| 540 | 2,4-difluorophenyl | methyl |
| 541 | 2,4-difluorophenyl | ethyl |
| 542 | 3,5-dichlorophenyl | methyl |
| 543 | 3,5-dichlorophenyl | ethyl |
| 544 | 4-fluorophenyl | n-propyl |
| 545 | 4-fluorophenyl | allyl |
| 546 | 4-fluorophenyl | isobutyl |
| 547 | 4-fluorophenyl | n-butyl |
| 548 | 2,6-difluorophenyl | Methyl |
| 549 | 2,3-difluorophenyl | methyl |
| 550 | 4-fluorophenyl | propargyl |
| 551 | 2,4-difluorophenyl | propargyl |
| 552 | 4-fluorophenyl | 2-trisfluoroethyl |

Specific compounds within the scope of this invention include the following. As used below, these compounds are named based on propionic acid derivatives but, alternatively, these compounds could have been named based on N-[2-N′,N′diethylamino-5-aminosulfonylphenylpyrimidin-4-yl]-p-carbomyloxy-phenylalanine derivatives.
2-{2-dimethylamino-5-[(4-chlorobenzenesulfonyl)methylamino]-pyrimidin-4-ylamino}-3-(4-dimethylcarbamoyloxyphenyl)propionic acid;
2-{2-dimethylamino-5-[(4-fluorobenzenesulfonyl)methylamino]-pyrimidin-4-ylamino}-3-(4-dimethylcarbamoyloxyphenyl)propionic acid;
2-{2-dimethylamino-5-[(3,4-difluorobenzenesulfonyl)methylamino]-pyrimidin-4-ylamino}-3-(4-dimethylcarbamoyloxyphenyl)propionic acid;
2-{2-dimethylamino-5-[(3,4-dichlorobenzenesulfonyl)methylamino]-pyrimidin-4-ylamino}-3-(4-dimethylcarbamoyloxyphenyl)propionic acid;
2-{2-dimethylamino-5-[(benzenesulfonyl)methylamino]-pyrimidin-4-ylamino}-3-(4-dimethylcarbamoyloxyphenyl)propionic acid;
2-{2-diethylamino-5-[(2-fluorobenzenesulfonyl)methylamino]-pyrimidin-4-ylamino}-3-(4-dimethylcarbamoyloxyphenyl)propionic acid;
2-{2-diethylamino-5-[(3-fluorobenzenesulfonyl)methylamino]-pyrimidin-4-ylamino}-3-(4-dimethylcarbamoyloxyphenyl)propionic acid;
2-{2-diethylamino-5-[(4-fluorobenzenesulfonyl)isopropylamino]-pyrimidin-4-ylamino}-3-(4-dimethylcarbamoyloxyphenyl)propionic acid;
2-{2-diethylamino-5-[(4-fluorobenzenesulfonyl)ethylamino]-pyrimidin-4-ylamino}-3-(4-dimethylcarbamoyloxyphenyl)propionic acid;
2-{2-diethylamino-5-[(3,4-difluorobenzenesulfonyl)isopropylamino]-pyrimidin-4-ylamino}-3-(4-dimethylcarbamoyloxyphenyl)propionic acid;
2-{2-diethylamino-5-[(4-chlorobenzenesulfonyl)isopropylamino]-pyrimidin-4-ylamino}-3-(4-dimethylcarbamoyloxyphenyl)propionic acid;
2-{2-diethylamino-5-[(3,4-difluorobenzenesulfonyl)ethylamino]-pyrimidin-4-ylamino}-3-(4-dimethylcarbamoyloxyphenyl)propionic acid;
2-{2-diethylamino-5-[(4-chlorobenzenesulfonyl)ethylamino]-pyrimidin-4-ylamino}-3-(4-dimethylcarbamoyloxyphenyl)propionic acid;
2-{2-diethylamino-5-[(4-fluorobenzenesulfonyl)cylclopropylmethyl-amino]pyrimidin-4-ylamino}-3-(4-dimethylcarbamoyloxyphenyl)propionic acid;
2-{2-diethylamino-5-[(3,5-difluorobenzenesulfonyl)methylamino]-pyrimidin-4-ylamino}-3-(4-dimethylcarbamoyloxyphenyl)propionic acid;
2-{2-diethylamino-5-[(3,5-difluorobenzenesulfonyl)ethylamino]-pyrimidin-4-ylamino}-3-(4-dimethylcarbamoyloxyphenyl)propionic acid;
2-{2-diethylamino-5-[(2,4-difluorobenzenesulfonyl)methylamino]-pyrimidin-4-ylamino}-3-(4-dimethylcarbamoyloxyphenyl)propionic acid;
2-{2-diethylamino-5-[(2,4-difluorobenzenesulfonyl)ethylamino]-pyrimidin-4-ylamino}-3-(4-dimethylcarbamoyloxyphenyl)propionic acid;
2-{2-diethylamino-5-[(3,5-dichlorobenzenesulfonyl)methylamino]-pyrimidin-4-ylamino}-3-(4-dimethylcarbamoyloxyphenyl)propionic acid;
2-{2-diethylamino-5-[(3,5-dichlorobenzenesulfonyl)ethylamino]-pyrimidin-4-ylamino}-3-(4-dimethylcarbamoyloxyphenyl)propionic acid;
2-{2-diethylamino-5-[(4-fluorobenzenesulfonyl)-n-propylamino]-pyrimidin-4-ylamino}-3-(4-dimethylcarbamoyloxyphenyl)propionic acid;
2-{2-diethylamino-5-[(4-fluorobenzenesulfonyl)allylamino]-pyrimidin-4-ylamino}-3-(4-dimethylcarbamoyloxyphenyl)propionic acid;
2-{2-diethylamino-5-[(4-fluorobenzenesulfonyl)isobotylamino]-pyrimidin-4-ylamino}-3-(4-dimethylcarbamoyloxyphenyl)propionic acid;
2-{2-diethylamino-5-[(4-fluorobenzenesulfonyl)-n-butylamino]-pyrimidin-4-ylamino}-3-(4-dimethylcarbamoyloxyphenyl)propionic acid;
2-{2-diethylamino-5-[(2,6-difluorobenzenesulfonyl)methylamino]-pyrimidin-4-ylamino}-3-(4-dimethylcarbamoyloxyphenyl)propionic acid;
2-{2-diethylamino-5-[(2,3-difluorobenzenesulfonyl)ethylamino]-pyrimidin-4-ylamino}-3-(4-dimethylcarbamoyloxyphenyl)propionic acid;
2-{2-Diethylamino-5-[(4-fluorobenzenesulfonyl)propargylamino] pyrimidin-4-ylamino}-3-(4-dimethylcarbamoyloxyphenyl)propionic acid;
2-{2-Diethylamino-5-[(2,4-difluorobenzenesulfonyl)propargylamino] pyrimidin-4-ylamino}-3-(4-dimethylcarbamoyloxyphenyl)propionic acid;
2-{2-Diethylamino-5-[(4-fluorobenzenesulfonyl)-(2-trisfluoroethyl)-amino]pyrimidin-4-ylamino}-3-(4-dimethylcarbamoyloxyphenyl)propionic acid;
   and pharmaceutically acceptable salts thereof.

The compounds of formulae XV-XX can be prepared from readily available starting materials using the methods and procedures set forth in the examples below. These methods and procedures outline specific reaction protocols for preparing N-[2-N′,N′-diethylamino-5-aminosulfonylphenyl-yrimidin-4-yl]-p-carbomyloxy-phenylalanine compounds. Compounds within the scope not exemplified in these examples and methods are readily prepared by appropriate substitution of starting materials which are either commercially available or well known in the art.

Other procedures and reaction conditions for preparing the compounds of this invention are described in the examples set forth below. Additionally, other procedures for preparing compounds useful in certain aspects of this invention are disclosed in U.S. Pat. No. 6,492,372, issued Dec. 10, 2002; the disclosure of which is incorporated herein by reference in its entirety.

Further description of the compounds of formulae XV-XX, procedures and reaction conditions for preparing these compounds are described in U.S. Patent Publication 2004/0142954, herein incorporated by reference in its entirety.

When describing the compounds of formulae VII-XX, compositions comprising compound of formulae VII-XX, and methods of this invention for compounds of formulae VII-XX, the following terms have the following meanings, unless otherwise indicated.

Definitions

As used herein, “lower alkyl” refers to monovalent alkyl groups having from 1 to 5 carbon atoms including straight and branched chain alkyl groups. This term is exemplified by groups such as methyl, ethyl, iso-propyl, n-propyl, n-butyl, iso-butyl, sec-butyl, t-butyl, n-pentyl and the like.

The term “lower alkylene” refers to divalent alkylene groups of from 1 to 4 carbon atoms including straight and branched chain alkylene groups. This term is exemplified by groups such as methylene, ethylene, n-propylene, iso-propylene (—CH₂CH(CH₃)— and —CH(CH₃)CH₂—) and the like.

The term “lower alkynyl” refers to an alkynyl group preferably having from 2 to 6 carbon atoms and having at least 1 site of alkynyl unsaturation (i.e., —C≡C). This term is exemplified by groups such as acetyl (—C≡CH), propargyl (—CH₂—C≡CH), 3-butynyl (—CH₂CH₂C≡CH₃) and the like.

“Propargyl” refers to the group —CH₂—C═CH.

The term “lower cycloalkyl” refers to cyclic alkyl groups of from 3 to 6 carbon atoms having a single cyclic ring including, by way of example, cyclopropyl, cyclobutyl, cyclopentyl and cyclohexyl.

The term “lower alkylenecycloalkyl” refers to the group consisting of a lower alkylene-lower cycloalkyl, as defined herein. Such groups are exemplified by methylenecyclopropyl (—CH₂-cyclopropyl), ethylenecyclopropyl and the like.

Compounds of Formulae XXI and XXIa

In another aspect, the compounds that can be utilized as steroid sparing agents for treatment a subject with inflammatory bowel disease, graft versus host disease, or host versus graft disease are compounds of the following formulae XXI and XXIa. Preferably, the compounds of formulae XXI and XXIa can be utilized as steriod sparing agents for treatment of a subject with inflammatory bowel disease.

In one aspect, the compounds that can be utilized as steroid sparing agents are compounds defined by formula XXI below. These compounds have a binding affinity to VLA-4 as expressed by an IC₅₀ of about 15 μM or less (as measured using the procedures described in Example A below). 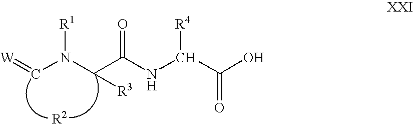
wherein:
R¹ is selected from the group consisting of alkyl, substituted alkyl, aryl, substituted aryl, cycloalkyl, substituted cycloalkyl, heterocyclic, substituted heterocylic, heteroaryl, substituted heteroaryl and —C(O)OR¹;
R² is selected from the group consisting of alkylene having from 2 to 4 carbon atoms in the alkylene chain, substituted alkylene having from 2 to 4 carbon atoms in the alkylene chain, heteroalkylene containing from 1 to 3 carbon atoms and from 1 to 2 heteroatoms selected from nitrogen, oxygen and sulfur and having from 2 to 4 atoms in the heteroalkylene chain, and substituted heteroalkylene containing, in the heteroalkylene chain, from 1 to 3 carbon atoms and from 1 to 2 heteroatoms selected from nitrogen, oxygen and sulfur and having from 2 to 4 atoms in the heteroalkylene chain;
R³ is selected from the group consisting of hydrogen, alkyl, substituted alkyl, cycloalkyl, substituted cycloalkyl, cycloalkenyl, substituted cycloalkenyl, aryl, substituted aryl, heteroaryl, substituted heteroaryl, heterocyclic, substituted heterocyclic; or R³ can be joined to R² to form a fused cycloalkyl, substititued cycloalkyl, cycloalkenyl, substituted cycloalkenyl, heterocyclic or substituted heterocyclic ring;
R⁴ is selected from the group consisting of isopropyl, —CH₂—X and ═CH—X, where X is selected from the group consisting of hydrogen, alkyl, substituted alkyl, cycloalkyl, substituted cycloalkyl, alkoxy, substituted alkoxy, aryl, substituted aryl, aryloxy, substituted aryloxy, aryloxyaryl, substituted aryloxyaryl, heteroaryl, substituted heteroaryl, heterocyclic, substituted heterocyclic, acylamino, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxyheterocyclic, carboxy-substituted heterocyclic, and hydroxyl with the proviso that when R⁴ is ═CH—X then (H) is removed from the formula and X is not hydroxyl;
W is oxygen or sulfur;
and pharmaceutically acceptable salts thereof.

In another embodiment, the compounds of formula XXI can also be provided as prodrugs which convert (e.g., hydrolyze, metabolize, etc.) in vivo to a compound of formula XXI above. In a preferred example of such an embodiment, the carboxylic acid in the compound of formula XXI is modified into a group which, in vivo, will convert to the carboxylic acid (including salts thereof). In a particularly preferred embodiment, such prodrugs are represented by compounds of formula XXIa: 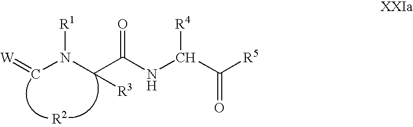
wherein:
R¹ is selected from the group consisting of alkyl, substituted alkyl, aryl, substituted aryl, cycloalkyl, substituted cycloalkyl, heterocyclic, substituted heterocylic, heteroaryl, substituted heteroaryl and —C(O)OR¹;
R² is selected from the group consisting of alkylene having from 2 to 4 carbon atoms in the alkylene chain, substituted alkylene having from 2 to 4 carbon atoms in the alkylene chain, heteroalkylene containing from 1 to 3 carbon atoms and from 1 to 2 heteroatoms selected from nitrogen, oxygen and sulfur and having from 2 to 4 atoms in the heteroalkylene chain, and substituted heteroalkylene containing, in the heteroalkylene chain, from 1 to 3 carbon atoms and from 1 to 2 heteroatoms selected from nitrogen, oxygen and sulfur and having from 2 to 4 atoms in the heteroalkylene chain;
R³ is selected from the group consisting of hydrogen, alkyl, substituted alkyl, cycloalkyl, substituted cycloalkyl, cycloalkenyl, substituted cycloalkenyl, aryl, substituted aryl, heteroaryl, substituted heteroaryl, heterocyclic, substituted heterocyclic; or R³ can be joined to R² to form a fused cycloalkyl, substititued cycloalkyl, cycloalkenyl, substituted cycloalkenyl, heterocyclic or substituted heterocyclic ring;
R⁴ is selected from the group consisting of isopropyl, —CH₂—X and ═CH—X, where X is selected from the group consisting of hydrogen, alkyl, substituted alkyl, cycloalkyl, substituted cycloalkyl, alkoxy, substituted alkoxy, aryl, substituted aryl, aryloxy, substituted aryloxy, aryloxyaryl, substituted aryloxyaryl, heteroaryl, substituted heteroaryl, heterocyclic, substituted heterocyclic, acylamino, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxyheterocyclic, carboxy-substituted heterocyclic, and hydroxyl with the proviso that when R⁴ is ═CH—X then (H) is removed from the formula and X is not hydroxyl;
R⁵ is selected from the group consisting of amino, alkoxy, substituted alkoxy, cycloalkoxy, substituted cycloalkoxy, aryloxy, substituted aryloxy, heteroaryloxy, substituted heteroaryloxy, heterocyclyloxy, substituted heterocyclyloxy, —NHOY where Y is hydrogen, alkyl, substituted alkyl, aryl, or substituted aryl, and —NH(CH₂)ₚCOOY′ where Y′ is hydrogen, alkyl, substituted alkyl, aryl, or substituted aryl, and p is an integer of from 1 to 8;
W is oxygen or sulfur;
and pharmaceutically acceptable salts thereof;
with the provisos that:
(a) when R¹ is benzyl, R² is —CH₂CH₂—, R³ is hydrogen, R⁴ is benzyl, then R⁵ is not ethyl;
(b) when R¹ is 3,4-dichlorobenzyl, R² is —CH₂CH₂—, R³ is hydrogen, R⁴ is 4-(phenylcarbonylamino)benzyl, then R⁵ is not methyl;
(c) when R¹ is benzyl, R² is —CH₂CH₂—, R³ is hydrogen, R⁴ is 4-hydroxybenzyl, then R⁵ is not isopropyl or tert-butyl;
(d) when R¹ is 4-flurobenzyl, R² is —CH₂CH₂—, R³ is hydrogen, R⁵ is tert-butyl, then R⁴ is not 4-hydroxybenzyl or 4-(4-nitrophenoxy-carbonyloxy)benzyl;
(e) when R¹ is 4-cyanobenzyl, R² is —CH₂CH₂—, R³ is hydrogen, R⁴ is 4-hydroxybenzyl, then R⁵ is not tert-butyl; and
(f) when R¹ is benzyloxycarbonyl, R² is —NHCH₂—, R³ is hydrogen, R⁵ is tert-butyl, then R⁴ is not 4-hydroxybenzyl or 4-(N,N-dimethylcarbamyloxy)benzyl.

In a preferred embodiment, R¹ is a group having the formula: 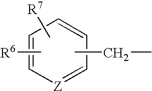
wherein:
R⁶ and R⁷ are independently selected from the group consisting of hydrogen, alkyl, alkoxy, amino, cyano, halo and nitro; and
Z is CH or N.

Preferably, Z is CH.

Preferably, one of R⁶ and R⁷ is hydrogen and the other is selected from the group consisting of hydrogen, methyl, methoxy, amino, chloro, fluoro, cyano or nitro; or both R⁶ and R⁷ are chloro.

In a particularly preferred embodiment, R¹ is selected from the group consisting of benzyl, 4-aminobenzyl, 3-chlorobenzyl, 4-chlorobenzyl, 3,4-dichlorobenzyl, 4-cyanobenzyl, 4-fluorobenzyl, 4-methylbenzyl, 4-methoxybenzyl, 4-nitrobenzyl, benzyloxycarbonyl, (pyrdin-3-yl)methyl and the like.

Preferably, R² is selected from the group consisting of alkylene having 2 or 3 carbon atoms in the alkylene chain, substituted alkylene having 2 or 3 carbon atoms in the alkylene chain, heteroalkylene containing 1 or 2 carbon atoms and 1 heteroatom selected from nitrogen, oxygen and sulfur and having 2 or 3 atoms in the heteroalkylene chain, and substituted heteroalkylene containing, in the heteroalkylene chain, 1 or 2 carbon atoms and 1 heteroatom selected from nitrogen, oxygen and sulfur and having 2 or 3 atoms in the heteroalkylene chain.

In a particularly preferred embodiment, R² is selected from the group consisting of —CH₂CH₂—, —CH₂—S—CH₂—, —CH₂—O—CH₂— and —NHCH₂—. Accordingly, R² when joined with the other atoms of the nitrogen-containing ring structure preferably forms a 2-pyrrolidinone, 3-oxothiomorpholine, 3-oxomorpholine or 2-imidazolidinone ring. In another preferred embodiment, R³ is joined to R² to form a 5-oxo-4-azatricyclo[4.2.1.0 (3,7)]nonane ring.

Preferably, in the compounds of formulae XXI and Ia above, R³ is hydrogen or it is joined with R² to form a 5-oxo-4-azatricyclo[4.2.1.0 (3,7)]nonane ring. More preferably, R³ is hydrogen.
R⁴ is preferably selected from all possible isomers arising by substitution with the following groups:
4-methylbenzyl,
4-hydroxybenzyl,
4-methoxybenzyl,
4-t-butoxybenzyl,
4-benzyloxybenzyl,
4-[φ-CH(CH₃)O-]benzyl,
4-[φ-CH(COOH)O-]benzyl,
4-[BocNHCH₂C(O)NH-]benzyl,
4-chlorobenzyl,
4-[NH₂CH₂C(O)NH-]benzyl,
4-carboxybenzyl,
4-[CbzNHCH₂CH₂NH-]benzyl,
3-hydroxy-4-(φ-OC(O)NHbenzyl,
4-[HOOCCH₂CH₂C(O)NH-]benzyl,
benzyl,
4-[2′-carboxylphenoxy-]benzyl,
4-[φ-C(O)NH-]benzyl,
3-carboxybenzyl,
4-iodobenzyl,
4-hydroxy-3,5-diiodobenzyl,
4-hydroxy-3-iodobenzyl,
4-[2′-carboxyphenyl-]benzyl,
φ-CH₂CH₂—,
4-nitrobenzyl,
2-carboxybenzyl,
4-[dibenzylamino]-benzyl,
4-[(1′-cyclopropylpiperidin-4′-yl)C(O)NH-]benzyl,
4-[-NHC(O)CH₂NHBoc]benzyl,
4-carboxybenzyl,
4-hydroxy-3-nitrobenzyl,
4-[-NHC(O)CH(CH₃)NHBoc]benzyl,
4-[-NHC(O)CH(CH₂φ)NHBoc]benzyl,
isobutyl,
methyl,
4-[CH₃C(O)NH-]benzyl,
—CH₂-(3-indolyl),
n-butyl,
t-butyl-OC(O)CH₂—,
t-butyl-OC(O)CH₂CH₂—,
H₂NC(O)CH₂—,
H₂NC(O)CH₂CH₂—,
BocNH—(CH₂)₄—,
t-butyl-OC(O)—(CH₂)₂—,
HOOCCH₂—,
HOOC(CH₂)₂—,
H₂N(CH₂)₄—,
isopropyl,
(1-naphthyl)-CH₂—,
(2-naphthyl)-CH₂—,
(2-thiophenyl)-CH₂—,
(φ-CH₂—OC(O)NH—(CH₂)₄—,
cyclohexyl-CH₂—,
benzyloxy-CH₂—,
HOCH₂—,
5-(3-N-benzyl)imidazolyl-CH₂—,
2-pyridyl-CH₂—,
3-pyridyl-CH₂—,
4-pyridyl-CH₂—,
5-(3-N-methyl)imidazolyl-CH₂—,
N-benzylpiperid-4-yl-CH₂—,
N-Boc-piperidin-4-yl-CH₂—,
N-(phenyl-carbonyl)piperidin-4-yl-CH₂—,
H₃CSCH₂CH₂—,
1-N-benzylimidazol-4-yl-CH₂—,
iso-propyl-C(O)NH—(CH₂)₄—,
iso-butyl-C(O)NH—(CH₂)₄—,
phenyl-C(O)NH—(CH₂)₄—,
benzyl-C(O)NH—(CH₂)₄—,
allyl-C(O)NH—(CH₂)₄—,
4-(3-N-methylimidazolyl)-CH₂—,
4-imidazolyl,
4-[(CH₃)₂NCH₂CH₂CH₂—O-]benzyl,
4-[(benzyl)₂N-]-benzyl,
4-aminobenzyl,
allyloxy-C(O)NH(CH₂)₄—,
allyloxy-C(O)NH(CH₂)₃—,
allyloxy-C(O)NH(CH₂)₂—,
NH₂C(O)CH₂—,
φ-CH═,
2-pyridyl-C(O)NH—(CH₂)₄—,
4-methylpyrid-3-yl-C(O)NH—(CH₂)₄—,
3-methylthien-2-yl-C(O)NH—(CH₂)₄—,
2-pyrrolyl-C(O)NH—(CH₂)₄—,
2-furanyl-C(O)NH—(CH₂)₄—,
4-methylphenyl-SO₂—N(CH₃)CH₂C(O)NH(CH₂)₄—,
4-[cyclopentylacetylenyl]-benzyl,
4-[-NHC(O)-(N-Boc)-pyrrolidin-2-yl)]-benzyl-,
1-N-methylimidazol-4-yl-CH₂—,
1-N-methylimidazol-5-yl-CH₂—,
imidazol-5-yl-CH₂—,
6-methylpyrid-3-yl-C(O)NH—(CH₂)₄—,
4-[2′-carboxymethylphenyl]-benzyl,
4-[-NHC(O)NHCH₂CH₂CH₂-φ]-benzyl,
4-[-NHC(O)NHCH₂CH₂-φ]-benzyl,
—CH₂C(O)NH(CH₂)₄P,
4-[φ(CH₂)₄O-]-benzyl,
4-[-C≡C-φ-4′φ]-benzyl,
4-[-C≡C—CH₂—O—S(O)₂-4′—CH₃-φ]-benzyl,
4-[-C≡C—CH₂NHC(O)NH₂]-benzyl,
4-[-C C—CH₂—O-4′—COOCH₂CH₃-φ]-benzyl,
4-[-C≡C—CH(NH₂)-cyclohexyl]-benzyl,
—(CH₂)₄NHC(O)CH₂-3-indolyl,
—(CH₂)₄NHC(O)CH₂CH₂-3-indolyl,
—(CH₂)₄NHC(O)-3-(5-methoxyindolyl),
—(CH₂)₄NHC(O)-3-(1-methylindolyl),
—(CH₂)₄NHC(O)-4-(-SO₂(CH₃)-φ),
—(CH₂)₄NHC(O)-4-(C(O)CH₃)-phenyl,
—(CH₂)₄NHC(O)-4-fluorophenyl,
—(CH₂)₄NHC(O)CH₂O-4-fluorophenyl,
4-[-C≡C-(2-pyridyl)]benzyl,
4-[-C≡C—CH₂—O-phenyl]benzyl,
4-[-C≡C—CH₂OCH₃]benzyl,
4-[-C≡C-(3-hydroxyphenyl)]benzyl,
4-[-C≡C—CH₂—O-4′-(-C(O)OC₂H₅)phenyl]benzyl,
4-[-C≡C—CH₂CH(C(O)OCH₃)₂]benzyl,
4-[-C≡C—CH₂NH-(4,5-dihydro-4-oxo-5-phenyl-oxazol-2-yl),
3-aminobenzyl,
4-[-C≡C—CH₂CH(NHC(O)CH₃)C(O)OH]-benzyl,
—CH₂C(O)NHCH(CH₃)φ,
—CH₂C(O)NHCH₂-(4-dimethylamino)-φ,
—CH₂C(O)NHCH₂-4-nitrophenyl,
—CH₂CH₂C(O)N(CH₃)CH₂-φ,
—CH₂CH₂C(O)NHCH₂CH₂-(N-methyl)-2-pyrrolyl,
—CH₂CH₂C(O)NHCH₂CH₂CH₂CH₃,
—CH₂CH₂C(O)NHCH₂CH₂-3-indolyl,
—CH₂C(O)N(CH₃)CH₂phenyl,
—CH₂C(O)NH(CH₂)₂-(N-methyl)-2-pyrrolyl,
—CH₂C(O)NHCH₂CH₂CH₂CH₃,
—CH₂C(O)NHCH₂CH₂-3-indolyl,
—(CH₂)₂C(O)NHCH(CH₃)φ,
—(CH₂)₂C(O)NHCH₂-4-dimethylaminophenyl,
—(CH₂)₂C(O)NHCH₂-4-nitrophenyl,
—CH₂C(O)NH-4-[-NHC(O)CH₃-phenyl],
—CH₂C(O)NH-4-pyridyl,
—CH₂C(O)NH-4-[dimethylaminophenyl],
—CH₂C(O)NH-3-methoxyphenyl,
—CH₂CH₂C(O)NH-4-chlorophenyl,
—CH₂CH₂C(O)NH-2-pyridyl,
—CH₂CH₂C(O)NH-4-methoxyphenyl,
—CH₂CH₂C(O)NH-3-pyridyl,
4-[(CH₃)₂NCH₂CH₂O-]benzyl,
—(CH₂)₃NHC(NH)NH—SO₂-4-methylphenyl,
4-[(CH₃)₂NCH₂CH₂O-]benzyl,
—(CH₂)₄NHC(O)NHCH₂CH₃,
—(CH₂)₄NHC(O)NH-phenyl,
—(CH₂)₄NHC(O)NH-4-methoxyphenyl,
4-[4′-pyridyl-C(O)NH-]benzyl,
4-[3′-pyridyl-C(O)NH-]benzyl,
4-[-NHC(O)NH-3′-methylphenyl]benzyl,
4-[-NHC(O)CH₂NHC(O)NH-3′-methylphenyl]benzyl,
4-[-NHC(O)-(2′,3′-dihydroindol-2-yl)]benzyl,
4-[-NHC(O)-(2′,3′-dihydro-N-Boc-indol-2-yl)]benzyl,
p-[—OCH₂CH₂-1′-(4′-pyrimidinyl)-piperazinyl]benzyl,
4-[—OCH₂CH₂-(1′-piperidinyl)benzyl,
4-[—OCH₂CH₂-(1′-pyrrolidinyl)]benzyl,
4-[—OCH₂CH₂CH₂-(1′-piperidinyl)]benzyl-,
40—CH₂-3-(1,2,4-triazolyl),
4-[—OCH₂CH₂CH₂-4-(3′-chlorophenyl)-piperazin-1-yl]benzyl,
4-[—OCH₂CH₂N(φ)CH₂CH₃]benzyl,
4-[—OCH₂-3′-(N-Boc)-piperidinyl]benzyl,
4-[di-n-pentylamino]benzyl,
4-[n-pentylamino]benzyl,
4-[di-iso-propylamino-CH₂CH₂O-]benzyl,
4-[—OCH₂CH₂-(N-morpholinyl)]benzyl,
4-[—O—(3′-(N-Boc)-piperidinyl]benzyl,
4-[—OCH₂CH(NHBoc)CH₂cyclohexyl]benzyl,
p-[OCH₂CH₂-(N-piperidinyl]benzyl,
4-[—OCH₂CH₂CH₂-(4-m-chlorophenyl)-piperazin-1-yl]benzyl,
4-[—OCH₂CH₂-(N-homopiperidinyl)benzyl,
4-[-NHC(O)-3′-(N-Boc)-piperidinyl]benzyl,
4-[—OCH₂CH₂N(benzyl)₂]benzyl,
—CH₂-2-thiazolyl,
3-hydroxybenzyl,
4-[—OCH₂CH₂CH₂N(CH₃)₂]benzyl,
4-[-NHC(S)NHCH₂CH₂-(N-morpholino)]benzyl,
4-[—OCH₂CH₂N(C₂H₅)₂]benzyl,
4-[—OCH₂CH₂CH₂N(C₂H₅)₂]benzyl,
4-[CH₃(CH₂)₄NH-]benzyl,
4-[N-n-butyl,N-n-pentylamino-]benzyl,
4-[-NHC(O)-4′-piperidinyl]benzyl,
4-[-NHC(O)CH(NHBoc)(CH₂)₄NHCbz]benzyl,
4-[-NHC(O)-(1′,2′,3′,4′-tetrahydro-N-Boc-isoquinolin-1′-yl]benzyl,
p-[—OCH₂CH₂CH₂-1′-(4′-methyl)-piperazinyl]benzyl,
—(CH₂)₄NH-Boc,
3-[—OCH₂CH₂CH₂N(CH₃)₂]benzyl,
4-[—OCH₂CH₂CH₂N(CH₃)₂]benzyl,
3-[—OCH₂CH₂-(1′-pyrrolidinyl)]benzyl,
4-[—OCH₂CH₂CH₂N(CH₃)benzyl]benzyl,
4-[-NHC(S)NHCH₂CH₂CH₂-(N-morpholino)]benzyl,
4-[—OCH₂CH₂-(N-morpholino)]benzyl,
4-[-NHCH₂-(4′-chlorophenyl)]benzyl,
4-[-NHC(O)NH-(4′-cyanophenyl)]benzyl,
4-[—OCH₂COOH]benzyl,
4-[—OCH₂COO-t-butyl]benzyl,
4-[-NHC(O)-5′-fluoroindol-2-yl]benzyl,
4-[-NHC(S)NH(CH₂)₂-1-piperidinyl]benzyl,
4-[-N(SO₂CH₃)(CH₂)₃—N(CH₃)₂]benzyl,
4-[-NHC(O)CH₂CH(C(O)OCH₂φ)-NHCbz]benzyl,
4-[-NHS(O)₂CF₃]benzyl,
3-[—O—(N-methylpiperidin-4′-yl]benzyl,
4-[-C(═NH)NH₂]benzyl,
4-[-NHSO₂—CH₂Cl]benzyl,
4-[-NHC(O)-(1′,2′,3′,4′-tetrahydroisoquinolin-2′-yl]benzyl,
4-[-NHC(S)NH(CH₂)₃-N-morpholino]benzyl,
4-[-NHC(O)CH(CH₂CH₂CH₂CH₂NH₂)NHBoc]benzyl,
4-[-C(O)NH₂]benzyl,
4-[-NHC(O)NH-3′-methoxyphenyl]benzyl,
4-[—OCH₂CH₂-indol-3′-yl]benzyl,
4-[—OCH₂C(O)NH-benzyl]benzyl,
4-[—OCH₂C(O)O-benzyl]benzyl,
4-[—OCH₂C(O)OH]benzyl,
4-[—OCH₂-2′-(4′,5′-dihydro)imidazolyl]benzyl,
—CH₂C(O)NHCH₂-(4-dimethylamino)phenyl,
—CH₂C(O)NHCH₂-(4-dimethylamino)phenyl,
4-[-NHC(O)-L-2′-pyrrolidinyl-N—SO₂-4′-methylphenyl]benzyl,
4-[-NHC(O)NHCH₂CH₂CH₃]benzyl,
4-aminobenzyl]benzyl,
4-[—OCH₂CH₂-1-(4-hydroxy-4-(3-methoxypyrrol-2-yl)-piperazinyl]benzyl,
4-[—O—(N-methylpiperidin-4′-yl)]benzyl,
3-methoxybenzyl,
4-[-NHC(O)-piperidin-3′-yl]benzyl,
4-[-NHC(O)-pyridin-2′-yl]benzyl,
4-[-NHCH₂-(4′-chlorophenyl)]benzyl,
4-[-NHC(O)-(N-(4′—CH₃-φ-SO₂)-L-pyrrolidin-2′-yl)]benzyl,
4-[-NHC(O)NHCH₂CH₂-φ]benzyl,
4-[—OCH₂C(O)NH₂]benzyl,
4-[—OCH₂C(O)NH-t-butyl]benzyl,
4-[—OCH₂CH₂-1-(4-hydroxy-4-phenyl)-piperidinyl]benzyl,
4-[-NHSO₂—CH═CH₂]benzyl,
4-[-NHSO₂—CH₂CH₂Cl]benzyl,
—CH₂C(O)NHCH₂CH₂N(CH₃)₂,
4-[(1′—Cbz-piperidin-4′-yl)C(O)NH-]benzyl,
4-[(1′-Boc-piperidin-4′-yl)C(O)NH-]benzyl,
4-[(2′-bromophenyl)C(O)NH-]benzyl,
4-[-NHC(O)-pyridin-4′-yl]benzyl,
4-[(4′-(CH₃)₂NC(O)O-)phenyl)-C(O)NH-]benzyl,
4-[-NHC(O)-1′-methylpiperidin-4′-yl-]benzyl,
4-(dimethylamino)benzyl,
4-[-NHC(O)-(1′-N-Boc)-piperidin-2′-yl]benzyl,
3-[-NHC(O)-pyridin-4′-yl]benzyl,
4-[(tert-butyl-O(O)CCH₂—O-benzyl)-NH-]benzyl,
[BocNHCH₂C(O)NH-]butyl,
4-benzylbenzyl,
2-hydroxyethyl,
4-[(Et)₂NCH₂CH₂CH₂NHC(S)NH-]benzyl,
4-[(1′-Boc-4′-hydroxypyrrolidin-2′-yl)C(O)NH-]benzyl,
4-[(φCH₂CH₂CH₂NHC(S)NH-]benzyl,
4-[(perhydroindolin-2′-yl)C(O)NH-]benzyl,
2-[4-hydroxy-4-(3-methoxythien-2-yl)piperidin-1-yl]ethyl,
4-[(1′-Boc-perhydroindolin-2′-yl)-C(O)NH-]benzyl,
4-[N-3-methylbutyl-N-trifluoromethanesulfonyl)amino]benzyl,
4-[N-vinylsulfonyl)amino]benzyl,
4-[2-(2-azabicyclo[3.20.2]octan-2-yl)ethyl-O-]benzyl,
4-[4′-hydroxypyrrolidin-2′-yl)C(O)NH-]benzyl,
4-(φNHC(S)NH)benzyl,
4-(EtNHC(S)NH)benzyl,
4-(φCH₂NHC(S)NH)benzyl,
3-[(1′-Boc-piperidin-2′-yl)C(O)NH-]benzyl,
3-[piperidin-2′-yl-C(O)NH-]benzyl,
4-[(3′-Boc-thiazolidin-4′-yl)C(O)NH-]benzyl,
4-(pyridin-3′-yl-NHC(S)NH)benzyl,
4-(CH₃—NHC(S)NH)benzyl,
4-(H₂NCH₂CH₂CH₂C(O)NH)benzyl,
4-(BOCHNCH₂CH₂CH₂C(O)NH)benzyl,
4-(pyridin-4′-yl-CH₂NH)benzyl,
4-[(N,N-di(4-N,N-dimethylamino)benzyl)amino]benzyl,
4-[(1—Cbz-piperidin-4-yl)C(O)NH-]butyl,
4-[(φCH₂OCH₂(BocHN)CHC(O)NH]benzyl,
4-[(piperidin-4′-yl)C(O)NH-]benzyl,
4-[(pyrrolidin-2′-yl)C(O)NH-]benzyl,
4-(pyridin-3′-yl-C(O)NH)butyl,
4-(pyridin-4′-yl-C(O)NH)butyl,
4-(pyridin-3′-yl-C(O)NH)benzyl,
4-[CH₃NHCH₂CH₂CH₂C(O)NH-]benzyl,
4-[CH₃N(Boc)CH₂CH₂CH₂C(O)NH-]benzyl,
4-(aminomethyl)benzyl,
4-[(φCH₂OCH₂(H₂N)CHC(O)NH]benzyl,
4-[(1′,4′-di(Boc)piperazin-2′-yl)-C(O)NH-]benzyl,
4-[(piperazin-2′-yl)-C(O)NH-]benzyl,
4-[(N-toluenesulfonylpyrrolidin-2′-yl)C(O)NH-]butyl,
4-[-NHC(O)-4′-piperidinyl]butyl,
4-[-NHC(O)-1′-N-Boc-piperidin-2′-yl]benzyl,
4-[-NHC(O)-piperidin-2′-yl]benzyl,
4-[(1′-N-Boc-2′,3′-dihydroindolin-2′-yl)-C(O)NH]benzyl,
4-(pyridin-3′-yl-CH₂NH)benzyl,
4-[(piperidin-1′-yl)C(O)CH₂—O-]benzyl,
4-[(CH₃)₂CH)₂NC(O)CH₂—O-]benzyl,
4-[HO(O)C(Cbz-NH)CHCH₂CH₂—C(O)NH-]benzyl,
4-[(φCH₂O(O)C(Cbz-NH)CHCH₂CH₂—C(O)NH-]benzyl,
4-[-NHC(O)-2′-methoxyphenyl]benzyl,
4-[(pyrazin-2′-yl)C(O)NH-]benzyl,
4-[HO(O)C(NH₂)CHCH₂CH₂—C(O)NH-]benzyl,
4-(2′-formyl-1′,2′,3′,4′-tetrahydroisoquinolin-3′-yl-CH₂NHbenzyl,
N-Cbz-NHCH₂—,
4-[(4′-methylpiperazin-1′-yl)C(O)O-]benzyl,
4-[CH₃(N-Boc)NCH₂C(O)NH-]benzyl,
4-[-NHC(O)-(1′,2′,3′,4′-tetrahydro-N-Boc-isoquinolin-3′-yl]-benzyl,
4-[CH₃NHCH₂C(O)NH-]benzyl,
(CH₃)₂NC(O)CH₂—,
4-(N-methylacetamido)benzyl,
4-(1′,2′,3′,4′-tetrahydroisoquinolin-3′-yl-CH₂NHbenzyl,
4-[(CH₃)₂NHCH₂C(O)NH-]benzyl,
(1-toluenesulfonylimidizol-4-yl)methyl,
4-[(1′-Boc-piperidin-4′-yl)C(O)NH-]benzyl,
4-trifluoromethylbenzyl,
4-[(2′-bromophenyl)C(O)NH-]benzyl,
4-[(CH₃)₂NC(O)NH-]benzyl,
4-[CH₃₀C(O)NH-]benzyl,
4-[(CH₃)₂NC(O)O-]benzyl,
4-[(CH₃)₂NC(O)N(CH₃)-]benzyl,
4-[CH₃₀C(O)N(CH₃)-]benzyl,
4-(N-methyltrifluoroacetamido)benzyl,
4-[(1′-methoxycarbonylpiperidin-4′-yl)C(O)NH-]benzyl,
4-[(4′-phenylpiperidin-4′-yl)C(O)NH-]benzyl,
4-[(4′-phenyl-1′-Boc-piperidin-4′-yl)-C(O)NH-]benzyl,
4-[(piperidin-4′-yl)C(O)O-]benzyl, 4-[(1′-methylpiperidin-4′-yl)-O-]benzyl,
4-[(1′-methylpiperidin-4′-yl)C(O)O-]benzyl,
4-[(4′-methylpiperazin-1′-yl)C(O)NH-]benzyl,
3-[(CH₃)₂NC(O)O-]benzyl,
4-[(4′-phenyl-1′-Boc-piperidin-4′-yl)-C(O)O-]benzyl,
4-(N-toluenesulfonylamino)benzyl,
4-[(CH₃)₃CC(O)NH-]benzyl,
4-[(morpholin-4′-yl)C(O)NH-]benzyl,
4-[(CH₃CH₂)₂NC(O)NH-]benzyl,
4-[-C(O)NH-(4′-piperidinyl)]benzyl,
4-[(2′-trifluoromethylphenyl)C(O)NH-]benzyl,
4-[(2′-methylphenyl)C(O)NH-]benzyl,
4-[(CH₃)₂NS(O)₂O-]benzyl,
4-[(pyrrolidin-2′-yl)C(O)NH-]benzyl,
4-[-NHC(O)-piperidin-1′-yl]benzyl,
4-[(thiomorpholin-4′-yl)C(O)NH-]benzyl,
4-[(thiomorpholin-4′-yl sulfone)-C(O)NH-]benzyl,
4-[(morpholin-4′-yl)C(O)O-]benzyl,
3-nitro-4-(CH₃₀C(O)CH₂Obenzyl,
(2-benzoxazolinon-6-yl)methyl-,
(2H-1,4-benzoxazin-3(4H)-one-7-yl)methyl-,
4-[(CH₃)₂NS(O)₂NH-]benzyl,
4-[(CH₃)₂NS(O)₂N(CH₃)-]benzyl,
4-[(thiomorpholin-4′-yl)C(O)O-]benzyl,
4-[(thiomorpholin-4′-yl sulfone)-C(O)O-]benzyl,
4-[(piperidin-1′-yl)C(O)O-]benzyl,
4-[(pyrrolidin-1′-yl)C(O)O-]benzyl,
4-[(4′-methylpiperazin-1′-yl)C(O)O-]benzyl,
4-[(2′-methylpyrrolidin-1′-yl)-,
(pyridin-4-yl)methyl-,
4-[(piperazin-4′-yl)-C(O)O-]benzyl,
4-[(1′-Boc-piperazin-4′-yl)-C(O)O-]benzyl,
4-[(4′-acetylpiperazin-1′-yl)C(O)O-]benzyl,
p-[(4′-methanesulfonylpiperazin-1′-yl)-benzyl,
3-nitro-4-[(morpholin-4′-yl)-C(O)O-]benzyl,
4-{[(CH₃)₂NC(S)]₂N-}benzyl,
N-Boc-2-aminoethyl-,
4-[(1,1-dioxothiomorpholin-4-yl)-C(O)O-]benzyl,
4-[(CH₃)₂NS(O)₂-]benzyl,
4-(imidazolid-2′-one-1′-yl)benzyl,
4-[(piperidin-1′-yl)C(O)O-]benzyl,
1-N-benzyl-imidazol-4-yl-CH₂—,
3,4-dioxyethylenebenzyl,
3,4-dioxymethylenebenzyl,
4-[-N(SO₂)(CH₃)CH₂CH₂CH₂N(CH₃)₂]benzyl,
4-(3′-formylimidazolid-2′-one-1′-yl)benzyl,
4-[NHC(O)CH(CH₂CH₂CH₂CH₂NH₂)NHBoc]benzyl,
[2′-[4″-hydroxy-4″-(3′″-methoxythien-2′″-yl)piperidin-2″-yl]ethoxy]benzyl, and
p-[(CH₃)₂NCH₂CH₂N(CH₃)C(O)O-]benzyl.

In a preferred embodiment, R⁴ is preferably selected from all possible isomers arising by substitution with the following groups:
benzyl,
4-aminobenzyl,
4-hydroxybenzyl,
4-nitrobenzyl,
3-chloro-4-hydroxybenzyl,
4-(phenylC(O)NHbenzyl,
4-(pyridin-4-ylC(O)NHbenzyl,
4-[(CH₃)₂NC(O)O-]benzyl,
4-[(1′—Cbz-piperidin-4′-yl)C(O)NH-]benzyl,
4-[(piperidin-4′-yl)C(O)NH-]benzyl,
4-[—O—(N-methylpiperidin-4′-yl)]benzyl,
4-[(4′-methylpiperazin-1′-yl)C(O)O-]benzyl,
4-[(4′-(pyridin-2-yl)piperazin-1′-yl)C(O)O-]benzyl,
4-[(thiomorpholin-4′-yl)C(O)O-]benzyl,
3-chloro-4-[(CH₃)₂NC(O)O-]benzyl, and
5-(3-N-benzyl)imidazolyl-CH₂—.

In the compounds of formula XXIa, R⁵ is preferably 2,4-dioxo-tetrahydrofuran-3-yl (3,4-enol), methoxy, ethoxy, iso-propoxy, n-butoxy, t-butoxy, cyclopentoxy, neo-pentoxy, 2-α-iso-propyl-4—O-methylcyclohexoxy, 2—O-isopropyl-4-β-methylcyclohexoxy, —NH₂, benzyloxy, —NHCH₂COOH, —NHCH₂CH₂COOH, —NH-adamantyl, —NHCH₂CH₂COOCH₂CH₃, —NHSO₂-p-CH₃-φ, —NHOR⁸ where R⁸ is hydrogen, methyl, iso-propyl or benzyl, O—(N-succinimidyl), —O-cholest-5-en-3—O-yl, —OCH₂—OC(O)C(CH₃)₃, —O(CH₂)_{z}NHC(O)R⁹ where z is 1 or 2 and R⁹ is selected from the group consisting of pyrid-3-yl, N-methylpyridyl, and N-methyl-1,4-dihydro-pyrid-3-yl, —NR″C(O)—R′ where R′ is aryl, heteroaryl or heterocyclic and R″ is hydrogen or —CH₂C(O)OCH₂CH₃.

In the compounds of formulae XXI and XXIa above, W is preferably oxygen.

Preferred compounds within the scope of formulae XXI and XXIa above include by way of example:
N-(benzyl)-L-pyroglutamyl-L-phenylalanine
N-(benzyloxycarbonyl)-L-pyroglutamyl-L-phenylalanine
N-(benzyl)-L-pyroglutamyl-L-4-(phenylcarbonylamino)phenylalanine
N-(3,4-dichlorobenzyl)-L-pyroglutamyl-L-4-(phenylcarbonylamino)phenylalanine
N-(3-chlorobenzyl)-L-pyroglutamyl-L-4-(phenylcarbonylamino)phenylalanine
N-(3-chlorobenzyl)-L-pyroglutamyl-L-4-(phenylcarbonylamino)phenylalanine methyl ester
N-(4-chlorobenzyl)-L-pyroglutamyl-L-4-(phenylcarbonylamino)phenylalanine
N-(4-chlorobenzyl)-L-pyroglutamyl-L-4-(phenylcarbonylamino)phenylalanine methyl ester
N-(4-methylbenzyl)-L-pyroglutamyl-L-4-(phenylcarbonylamino)phenylalanine
N-(4-methylbenzyl)-L-pyroglutamyl-L-4-(phenylcarbonylamino)phenylalanine methyl ester
N-(4-methoxybenzyl)-L-pyroglutamyl-L-4-(phenylcarbonylamino)phenylalanine
N-(4-methoxybenzyl)-L-pyroglutamyl-L-4-(phenylcarbonylamino)phenylalanine methyl ester
N-(3-chlorobenzyl)-L-pyroglutamyl-L-(N′-benzyl)histidine
N-(4-methylbenzyl)-L-pyroglutamyl-L-(N′-benzyl)histidine methyl ester
N-(4-methylbenzyl)-L-pyroglutamyl-L-(N′-benzyl)histidine
N-(benzyl)-D-pyroglutamyl-L-phenylalanine
N-(4-benzyl-3-oxothiomorpholin-5-carbonyl)-L-phenylalanine
N-(4-benzyl-3-oxothiomorpholin-5-carbonyl)-L-phenylalanine ethyl ester
N-(4-benzyl-3-oxomorpholin-5-carbonyl)-L-phenylalanine
N-(4-benzyl-3-oxothiomorpholin-5-carbonyl)-L-4-nitrophenylalanine methyl ester
N-(benzyl)-L-pyroglutamyl-L-4-(pyridin-4-ylcarbonylamino)phenylalanine methyl ester
N-(benzyl)-L-pyroglutamyl-L-4-(1′-benzyloxycarbonylpiperidin-4′-ylcarbonylamino)phenylalanine methyl ester
N-(benzyl)-L-pyroglutamyl-L-4-(pyridin-4-ylcarbonylamino)phenylalanine
N-(benzyl)-L-pyroglutamyl-L-4-(1′-benzyloxycarbonylpiperidin-4′-ylcarbonylamino)phenylalanine
N-(benzyl)-L-pyroglutamyl-L-tyrosine ethyl ester
N-(benzyl)-L-pyroglutamyl-L-4-(piperidin-4′-ylcarbonylamino)phenylalanine
N-(benzyl)-L-pyroglutamyl-L-4-nitrophenylalanine ethyl ester
N-(benzyl)-L-pyroglutamyl-L-tyrosine
N-(benzyl)-L-pyroglutamyl-L-4-(1′-methylpiperidin-4′-yloxy)phenylalanine ethyl ester
N-(benzyl)-L-pyroglutamyl-L-4-nitrophenylalanine
N-(benzyl)-L-pyroglutamyl-L-4-[(4′-methylpiperazin-1′-yl)carbonyloxy]phenylalanine ethyl ester
N-(benzyl)-L-pyroglutamyl-L-4-(1′-methylpiperidin-4′-yloxy)phenylalanine
N-(benzyl)-L-pyroglutamyl-L-4-[(4′-methylpiperazin-1′-yl)carbonyloxy]phenylalanine
N-(benzyl)-L-pyroglutamyl-L-4-(N,N-dimethylcarbamyloxy)phenylalanine ethyl ester
N-(benzyl)-L-pyroglutamyl-L-4-aminophenylalanine ethyl ester
N-(benzyl)-L-pyroglutamyl-L-4-(N,N-dimethylcarbamyloxy)phenylalanine
N-(benzyl)-L-pyroglutamyl-L-4-(N,N-dimethylcarbamyloxy)phenylalanine tert-butyl ester
N-(benzyl)-L-pyroglutamyl-L-4-[(4′-methylpiperazin-1′-yl)carbonyloxy]phenylalanine tert-butyl ester
N-(benzyl)-L-pyroglutamyl-L-4-[(thiomorpholin-4′-yl)carbonyloxy]phenylalanine tert-butyl ester
N-(4-fluorobenzyl)-L-pyroglutamyl-L-4-[(thiomorpholin-4′-yl)carbonyloxy]phenylalanine tert-butyl ester
N-(benzyl)-L-pyroglutamyl-L-4-(N,N-dimethylcarbamyloxy)phenylalanine isopropyl ester
N-(4-fluorobenzyl)-L-pyroglutamyl-L-4-(N,N-dimethylcarbamyloxy)phenylalanine tert-butyl ester
N-(benzyl)-L-pyroglutamyl-L-3-chloro-4-hydroxyphenylalanine
N-(4-cyanobenzyl)-L-pyroglutamyl-L-4-(N,N-dimethylcarbamyloxy)phenylalanine tert-butyl ester
N-(benzyl)-L-pyroglutamyl-L-3-chloro-4-(N,N-dimethylcarbamyloxy)phenylalanine methyl ester
N-(4-fluorobenzyl)-L-pyroglutamyl-L-4-[(thiomorpholin-4′-yl)carbonyloxy]phenylalanine
N-(4-cyanobenzyl)-L-pyroglutamyl-L-4-(N,N-dimethylcarbamyloxy)phenylalanine
N-(1-benzyloxycarbonyl-2-imidazolidone-5-carbonyl)-L-4-(N,N-dimethylcarbamyloxy)phenylalanine
N-(4-nitrobenzyl)-L-pyroglutamyl-L-4-(N,N-dimethylcarbamyloxy)phenylalanine tert-butyl ester
N-(benzyl)-L-pyroglutamyl-L-3-chloro-4-(N,N-dimethylcarbamyloxy)phenylalanine
N-(4-fluorobenzyl)-L-pyroglutamyl-L-4-[(4′-(pyridin-2-yl)piperazin-1′-yl)carbonyloxy]phenylalanine
N-(4-fluorobenzyl)-L-pyroglutamyl-L-4-[(4′-(pyridin-2-yl)piperazin-1′-yl)carbonyloxy]phenylalanine tert-butyl ester
N-(4-aminobenzyl)-L-pyroglutamyl-L-4-(N,N-dimethylcarbamyloxy)phenylalanine tert-butyl ester
N-(pyridin-3-ylmethyl)-L-pyroglutamyl-L-tyrosine tert-butyl ester
N-(pyridin-3-ylmethyl)-L-pyroglutamyl-L-4-(N,N-dimethylcarbamyloxy)phenylalanine
N-(pyridin-3-ylmethyl)-L-pyroglutamyl-L-4-(N,N-dimethylcarbamyloxy)phenylalanine tert-butyl ester
N-(pyridin-3-ylmethyl)-L-pyroglutamyl-L-4-[(4′-(pyridin-2-yl)piperazin-1′-yl)carbonyloxy]phenylalanine tert-butyl ester
N-(pyridin-3-ylmethyl)-L-pyroglutamyl-L-4-[(4′-(pyridin-2-yl)piperazin-1′-yl)carbonyloxy]phenylalanine
N-(4-benzyl-5-oxo-4-azatricyclo[4.2.1.0 (3,7)]nonane-3-carbonyl)-L-tyrosine tert-butyl ester
N-(4-benzyl-5-oxo-4-azatricyclo[4.2.1.0 (3,7)]nonane-3-carbonyl)-L-4-(N,N-dimethylcarbamyloxy)phenylalanine tert-butyl ester
N-(4-benzyl-5-oxo-4-azatricyclo[4.2.1.0 (3,7)]nonane-3-carbonyl)-L-4-(N,N-dimethylcarbamyloxy)phenylalanine
N-(4-fluorobenzyl)-L-pyroglutamyl-L-4-(N,N-dimethylcarbamyloxy)phenylalanine
   and pharmaceutically acceptable salts thereof as well as any of the ester compounds recited above wherein one ester is replaced with another ester selected from the group consisting of methyl ester, ethyl ester, n-propyl ester, isopropyl ester, n-butyl ester, isobutyl ester, sec-butyl ester and tert-butyl ester.

Preferred compounds of formulae XXI and XXIa above include those set forth in Tables 7, 8, 9 and 10 below:

TABLE 7

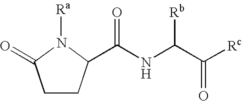

| R^{a} | R^{b} | R^{c} |
|---|---|---|
| φ-CH₂— | φ-CH₂— | —OH |
| φ-CH₂—OC(O)— | φ-CH₂— | —OH |
| φ-CH₂— | 4-[φ-C(O)NH-]-benzyl- | —OH |
| 3,4-dichlorobenzyl- | 4-[φ-C(O)NH-]-benzyl- | —OH |
| 4-chlorobenzyl- | 4-[φ-C(O)NH-]benzyl- | —OH |
| 3-chlorobenzyl- | 4-[φ-C(O)NH-]-benzyl- | —OCH₃ |
| 3-chlorobenzyl- | 4-[φ-C(O)NH-]-benzyl- | —OH |
| 4-chlorobenzyl- | 4-[φ-C(O)NH-]-benzyl- | —OCH₃ |
| 4-CH₃-benzyl- | 4-[φ-C(O)NH-]-benzyl- | —OCH₃ |
| 4-CH₃-benzyl- | 4-[φ-C(O)NH-]-benzyl- | —OH |
| 4-CH₃O-benzyl- | 4-[φ-C(O)NH-]-benzyl- | —OCH₃ |
| 4-CH₃O-benzyl- | 4-[φ-C(O)NH-]-benzyl- | —OH |
| 3-chlorobenzyl- | (1-benzylimidazol-4-yl)methyl- | —OH |
| 4-CH₃-benzyl- | (1-benzylimidazol-4-yl)methyl- | —OCH₃ |
| 4-CH₃-benzyl- | (1-benzylimidazol-4-yl)methyl- | —OH |
| φ-CH₂— | φ-CH₂— | —OH |
| φ-CH₂— | 4-[pyridin-4-yl-C(O)NH]-benzyl- | —OCH₃ |
| φ-CH₂— | 4-1[1-(benzyloxy-C(O)-)piperidin-4-yl-)C(O)NH-]-benzyl- | —OCH₃ |
| φ-CH₂— | 4-[pyridin-4-yl-C(O)NH]-benzyl- | —OH |
| φ-CH₂— | 4-[(1-(benzyloxy-C(O)-)piperidin-4-yl-)C(O)NH-]-benzyl- | —OH |
| φ-CH₂— | 4-hydroxybenzyl- | —OCH₂CH₃ |
| φ-CH₂— | 4-[pyridin-4-yl-C(O)NH]-benzyl- | —OH |
| φ-CH₂— | 4-NO₂-benzyl- | —OCH₂CH₃ |
| φ-CH₂— | 4-hydroxybenzyl- | —OH |
| φ-CH₂— | 4-[(1-methylpiperidin-4-yl-)O-]benzyl- | —OCH₂CH₃ |
| φ-CH₂— | 4-NO₂-benzyl- | —OH |
| φ-CH₂— | 4-[(4-methylpiperazin-1-yl-)C(O)O-]benzyl- | —OCH₂CH₃ |
| φ-CH₂— | 4-1(1-methylpiperidin-4-yl-)O-]benzyl- | —OH |
| φ-CH₂— | 4-[(4-methylpiperazin-1-yl-)C(O)O-]benzyl- | —OH |
| φ-CH₂— | 4-[(CH3)₂NC(O)O-]benzyl- | —OCH₂CH₃ |
| φ-CH₂— | 4-NH₂-benzyl- | —OCH₂CH₃ |
| φ-CH₂— | 4-[(CH₃)₂NC(O)O-]benzyl- | —OH |
| φ-CH₂— | 4-[(CH₃)₂NC(O)O-]benzyl- | —OC(CH₃)₃ |
| φ-CH₂— | 4-[(4-methylpiperazin-1-yl-)C(O)O-]benzyl- | —OC(CH₃)₃ |
| φ-CH₂— | 4-[thiomopholin-4-yl-C(O)O-]benzyl- | —OC(CH₃)₃ |
| 4-fluoro-benzyl- | 4-[thiomorpholin-4-yl-C(O)O-]benzyl- | —OC(CH₃)₃ |
| φ-CH₂— | 4-[(CH₃)₂NC(O)O-]benzyl- | —OCH(CH₃)₂ |
| 4-fluoro-benzyl- | 4-[(CH₃)₂NC(O)O-]benzyl- | —OC(CH₃)₃ |
| φ-CH₂— | 3-chloro-4-hydroxybenzyl- | —OCH₃ |
| 4-fluoro-benzyl- | 4-[(CH₃)₂NC(O)O-]benzyl- | —OC(CH₃)₃ |
| φ-CH₂— | 3-chloro-4-[(CH₃)₂NC(O)O-]benzyl- | —OCH₃ |
| 4-fluoro-benzyl- | 4-[(CH₃)₂NC(O)O-]benzyl- | —OH |
| 4-fluoro-benzyl- | 4-[thiomorpholin-4-yl-C(O)O-]benzyl- | —OH |
| 4-cyano-benzyl- | 4-[(CH₃)₂NC(O)O-]benzyl- | —OH |
| 4-NO₂-benzyl- | 4-[(CH₃)₂NC(O)O-]benzyl- | —OC(CH₃)₃ |
| φ-CH₂— | 3-chloro-4-[(CH₃)₂NC(O)O-]benzyl- | —OH |
| 4-fluoro-benzyl- | 4-[4-(pyridin-2-yl)piperazin-1-yl-C(O)O-]benzyl- | —OH |
| 4-fluoro-benzyl- | 4-[4-(pyridin-2-yl)piperazin-1-yl-C(O)O-]benzyl- | —OC(CH₃)₃ |
| 4-NH₂-benzyl- | 4-[(CH₃)₂NC(O)O-]benzyl- | —OC(CH₃)₃ |
| pyridin-3-CH₂— | 4-[(CH₃)₂NC(O)O-]benzyl- | —OH |
| pyridin-3-CH₂— | 4-[4-(pyridin-2-yl)piperazin-1-yl-C(O)O-]benzyl- | —OC(CH₃)₃ |
| pyridin-3-CH₂— | 4-[4-(pyridin-2-yl)piperazin-1-yl-C(O)O-]benzyl- | —OH |

TABLE 8

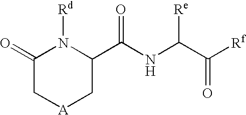

| R^{d} | R^{e} | R^{f} | A |
|---|---|---|---|
| φ-CH₂— | φ-CH₂— | —OH | S |
| φ-CH₂— | φ-CH₂— | —OCH₂CH₃ | S |
| φ-CH₂— | φ-CH₂— | —OH | O |
| φ-CH₂— | 4-NO₂-benzyl- | —OCH₃ | S |

TABLE 9

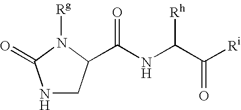

| R^{g} | R^{h} | Rⁱ |
|---|---|---|
| φ-CH₂-OC(O)— | 4-[(CH₃)₂NC(O)-]benzyl- | —OH |

TABLE 10

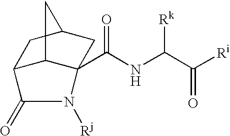

| R^{k} | R^{k} | R^{l} |
|---|---|---|
| φ-CH₂— | 4-hydroxybenzyl- | —OC(CH₃)₃ |
| φ-CH₂— | 4-[(CH₃)₂NC(O)-]benzyl- | —OC(CH₃)₃ |
| φ-CH₂— | 4-[(CH₃)₂NC(O)-]benzyl- | —OH |

The compounds of formulae XXI and XXIa can be prepared from readily available starting materials using the methods and procedures set forth in the examples below. These methods and procedures outline specific reaction protocols for preparing N-[2-N′,N′-diethylamino-5-aminosulfonylphenyl-yrimidin-4-yl]-p-carbomyloxy-phenylalanine compounds. Compounds within the scope not exemplified in these examples and methods are readily prepared by appropriate substitution of starting materials which are either commercially available or well known in the art.

Other procedures and reaction conditions for preparing the compounds of this invention are described in the examples set forth below. Additionally, other procedures for preparing compounds useful in certain aspects of this invention are disclosed in International Patent Application Publication No. WO 00/43413, published Jul. 27, 2000; the disclosure of which is incorporated herein by reference in its entirety.

When describing the compounds of formulae XXI and XXIa, compositions comprising compound of formulae XXI and XXIa, and methods of this invention for compounds of formulae XXI and XXIa, the following terms have the following meanings, unless otherwise indicated.

Definitions

As used herein, “alkyl” refers to alkyl groups preferably having from 1 to 10 carbon atoms and more preferably 1 to 6 carbon atoms. This term is exemplified by groups such as methyl, t-butyl, n-heptyl, octyl and the like.

“Substituted alkyl” refers to an alkyl group, preferably of from 1 to 10 carbon atoms, having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkylene” refers to a divalent hydrocarbon radical of the formula —(CH₂)ₙ— where n is an integer ranging from 1 to 10. By way of illustration, the term alkylene includes methylene (—CH₂—), ethylene (—CH₂CH₂—), propylene (—CH₂CH₂CH₂—) and the like.

“Substituted alkylene” refers to an alkylene group, preferably of from 1 to 10 carbon atoms, having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl. Additionally, two or more substituents on the substituted alkylene group may also be joined together to form a fused and/or bridged cycloalkyl, substituted cycloalkyl, heterocyclic or substituted heterocyclic group, or a fused aryl or heteroaryl group.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Alkoxycarbonyl” refers to the group “alkyl-O—C(O)—”.

“Substituted alkoxycarbonyl” refers to the group “substituted alkyl-O—C(O)—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocarbonylamino” refers to the group —C(S)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form, together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 10 carbon atoms and more preferably 2 to 6 carbon atoms and having at least 1 and preferably from 1-2 sites of alkenyl unsaturation.

“Substituted alkenyl” refers to alkenyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynyl” refers to alkynyl group preferably having from 2 to 10 carbon atoms and more preferably 3 to 6 carbon atoms and having at least 1 and preferably from 1-2 sites of alkynyl unsaturation.

“Substituted alkynyl” refers to alkynyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Amidino” refers to the group H₂NC(═NH)— and the term “alkylamidino” refers to compounds having 1 to 3 alkyl groups (e.g., alkyl-HNC(═NH)— and the like).

“Thioamidino” refers to the group RSC(═NH)— where R is hydrogen or alkyl.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the groups —NRC(O)O-alkyl, —NRC(O)O-substituted alkyl, —NRC(O)O-alkenyl, —NRC(O)O-substituted alkenyl, —NRC(O)O-alkynyl, —NRC(O)O-substituted alkynyl, —NRC(O)O-cycloalkyl, —NRC(O)O-substituted cycloalkyl, —NRC(O)O-aryl, —NRC(O)O-substituted aryl, —NRC(O)O-heteroaryl, —NRC(O)O-substituted heteroaryl, —NRC(O)O-heterocyclic, and —NRC(O)O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxycarbonylamino” refers to the groups —OC(O)NH₂, —OC(O)NRR, —OC(O)NR-alkyl, —OC(O)NR-substituted alkyl, —OC(O)NR-alkenyl, —OC(O)NR-substituted alkenyl, —OC(O)NR-alkynyl, —OC(O)NR-substituted alkynyl, —OC(O)NR-cycloalkyl, —OC(O)NR-substituted cycloalkyl, —OC(O)NR-aryl, —OC(O)NR-substituted aryl, —OC(O)NR-heteroaryl, —OC(O)NR-substituted heteroaryl, —OC(O)NR-heterocyclic, and —OC(O)NR-substituted heterocyclic where R is hydrogen, alkyl or where each R is joined to form, together with the nitrogen atom, a heterocyclic or substituted heterocyclic ring, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NH₂, —OC(S)NRR, —OC(S)NR-alkyl, —OC(S)NR-substituted alkyl, —OC(S)NR-alkenyl, —OC(S)NR-substituted alkenyl, —OC(S)NR-alkynyl, —OC(S)NR-substituted alkynyl, —OC(S)NR-cycloalkyl, —OC(S)NR-substituted cycloalkyl, —OC(S)NR-aryl, —OC(S)NR-substituted aryl, —OC(S)NR-heteroaryl, —OC(S)NR-substituted heteroaryl, —OC(S)NR-heterocyclic, and —OC(S)NR-substituted heterocyclic where R is hydrogen, alkyl or where each R is joined to form together with the nitrogen atom, a heterocyclic or substituted heterocyclic ring, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the groups —NRC(O)NRR, —NRC(O)NR-alkyl, —NRC(O)NR-substituted alkyl, —NRC(O)NR-alkenyl, —NRC(O)NR-substituted alkenyl, —NRC(O)NR-alkynyl, —NRC(O)NR-substituted alkynyl, —NRC(O)NR-aryl, —NRC(O)NR-substituted aryl, —NRC(O)NR-cycloalkyl, —NRC(O)NR-substituted cycloalkyl, —NRC(O)NR-heteroaryl, and —NRC(O)NR-substituted heteroaryl, —NRC(O)NR-heterocyclic, and —NRC(O)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the groups —NRC(S)NRR, —NRC(S)NR-alkyl, —NRC(S)NR-substituted alkyl, —NRC(S)NR-alkenyl, —NRC(S)NR-substituted alkenyl, —NRC(S)NR-alkynyl, —NRC(S)NR-substituted alkynyl, —NRC(S)NR-aryl, —NRC(S)NR-substituted aryl, —NRC(S)NR-cycloalkyl, —NRC(S)NR-substituted cycloalkyl, —NRC(S)NR-heteroaryl, and —NRC(S)NR-substituted heteroaryl, —NRC(S)NR-heterocyclic, and —NRC(S)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7yl, and the like). Preferred aryls include phenyl and naphthyl.

Substituted aryl refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, ——OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“4-Benzyl-5-oxo-4-azatricyclo[4.2.1.0 (3,7)]nonane-3-carboxylic acid” refers to a compound of the formula:

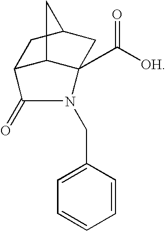

“Cycloalkyl” refers to cyclic alkyl groups of from 3 to 8 carbon atoms having a single cyclic ring including, by way of example, cyclopropyl, cyclobutyl, cyclopentyl, cyclooctyl and the like. Excluded from this definition are multi-ring or fused-ring alkyl groups such as adamantanyl, and the like.

“Cycloalkenyl” refers to cyclic alkenyl groups of from 3 to 8 carbon atoms having single or multiple unsaturation but which are not aromatic.

“Substituted cycloalkyl” and “substituted cycloalkenyl” refer to a cycloalkyl and cycloalkenyl groups, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkoxy” refers to —O-cycloalkyl groups.

“Substituted cycloalkoxy” refers to —O-substituted cycloalkyl groups.

“Guanidino” refers to the groups —NRC(═NR)NRR, —NRC(═NR)NR-alkyl, —NRC(═NR)NR-substituted alkyl, —NRC(═NR)NR-alkenyl, —NRC(═NR)NR-substituted alkenyl, —NRC(═NR)NR-alkynyl, —NRC(═NR)NR-substituted alkynyl, —NRC(═NR)NR-aryl, —NRC(═NR)NR-substituted aryl, —NRC(═NR)NR-cycloalkyl, —NRC(═NR)NR-heteroaryl, —NRC(═NR)NR-substituted heteroaryl, —NRC(═NR)NR-heterocyclic, and —NRC(═NR)NR-substituted heterocyclic where each R is independently hydrogen and alkyl as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Guanidinosulfone” refers to the groups —NRC(═NR)NRSO₂-alkyl, —NRC(═NR)NRSO₂-substituted alkyl, —NRC(═NR)NRSO₂-alkenyl, —NRC(═NR)NRSO₂-substituted alkenyl, —NRC(═NR)NRSO₂-alkynyl, —NRC(═NR)NRSO₂-substituted alkynyl, —NRC(═NR)NRSO₂-aryl, —NRC(═NR)NRSO₂-substituted aryl, —NRC(═NR)NRSO₂-cycloalkyl, —NRC(═NR)NRSO₂-substituted cycloalkyl, —NRC(═NR)NRSO₂-heteroaryl, and —NRC(═NR)NRSO₂-substituted heteroaryl, —NRC(═NR)NRSO₂-heterocyclic, and —NRC(═NR)NRSO₂-substituted heterocyclic where each R is independently hydrogen and alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Halo” or “halogen” refers to fluoro, chloro, bromo and iodo and preferably is either chloro or bromo.

“Heteroalkylene” refers to an alkylene group in which from 1 to 5, preferable from 1 to 3, of the carbon atoms in the alkylene chain have been replaced with a hetereoatom selected from nitrogen, oxygen or sulfur. By way of illustration, the term heteroalkylene includes —CH₂—O—CH₂—, —CH₂—S—CH₂—, —NHCH₂— and the like.

“Substituted heteroalkylene” refers to a heteroalkylene group having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Preferred heteroaryls include pyridyl, pyrrolyl, indolyl and furyl.

“Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from nitrogen, sulfur or oxygen within the ring wherein, in fused ring systems, one or more of the rings can be aryl or heteroaryl.

“Substituted heterocyclic” refers to heterocycle groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“L-Pyroglutamic acid” refers to (S)-(−)-2-pyrrolidone-5-carboxylic acid.

“Thiol” refers to the group —SH.

“Thioalkyl” refers to the groups —S-alkyl.

“Substituted thioalkyl” refers to the group —S-substituted alkyl.

“Thiocycloalkyl” refers to the groups —S-cycloalkyl.

“Substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

Pharmaceutical Formulations of the Compounds

In general, the compounds of the subject invention will be administered in a therapeutically effective amount by any of the accepted modes of administration for these compounds. The compounds can be administered by a variety of routes, including, but not limited to, oral, parenteral (e.g., subcutaneous, subdural, intravenous, intramuscular, intrathecal, intraperitoneal, intracerebral, intraarterial, or intralesional routes of administration), topical, intranasal, localized (e.g., surgical application or surgical suppository), rectal, and pulmonary (e.g., aerosols, inhalation, or powder). Accordingly, these compounds are effective as both injectable and oral compositions. The compounds can be administered continuously by infusion or by bolus injection. Preferably, the compounds are administered by parenteral routes. More preferably, the compounds are administered by intravenous routes. Such compositions are prepared in a manner well known in the pharmaceutical art.

The actual amount of the compound of the subject invention, i.e., the active ingredient, will depend on a number of factors, such as the severity of the disease, i.e., the condition or disease to be treated that is associated with steroid treatment which is desired to be tapered, the age and relative health of the subject, the potency of the compound used, the route and form of administration, and other factors.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The effective blood level of the compounds of the subject invention is preferably greater than or equal to 10 ng/ml.

The amount of the pharmaceutical composition administered to the patient will vary depending upon what is being administered, the purpose of the administration, such as prophylaxis or therapy, the state of the patient, the manner of administration, and the like. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described supra. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The active compound is effective over a wide dosage range and is generally administered in a pharmaceutically or therapeutically effective amount. The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 0.5 mg to about 100 mg per kilogram body weight, preferably about 3 mg to about 50 mg per kilogram body weight. Effective doses can be extrapolated from dose-response curves derived from in vitro or animal model test systems. Typically, the clinician will administer the compound until a dosage is reached that achieves the desired effect.

When employed as pharmaceuticals, the compounds of the subject invention are usually administered in the form of pharmaceutical compositions. This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient or enclosed within a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. The concentration of therapeutically active compound may vary from about 1 mg/ml to 250 g/ml.

Preferably, the compound can be formulated for parenteral administration in a suitable inert carrier, such as a sterile physiological saline solution. For example, the concentration of compound in the carrier solution is typically between about 1-100 mg/ml. The dose administered will be determined by route of administration. Preferred routes of administration include parenteral or intravenous administration. A therapeutically effective dose is a dose effective to produce a significant steroid tapering. Preferably, the amount is sufficient to produce a statistically significant amount of steroid tapering in a subject.

By way of example, for preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. The compositions may be administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The compounds of this invention can be administered in a sustained release form. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15: 167-277 (1981) and Langer, Chem. Tech. 12: 98-105 (1982) or poly(vinyl alcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e. injectable microspheres composed of lactic acid-glycolic acid copolymer and leuprolide acetate), and poly-D-(−)-3-hydroxybutyric acid (EP 133,988).

The compounds of this invention can be administered in a sustained release form, for example a depot injection, implant preparation, or osmotic pump, which can be formulated in such a manner as to permit a sustained release of the active ingredient. Implants for sustained release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host. The implant is placed in proximity to the site of protein deposits (e.g., the site of formation of amyloid deposits associated with neurodegenerative disorders), so that the local concentration of active agent is increased at that site relative to the rest of the body.

The following formulation examples illustrate pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/capsule) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### Formulation Example 4

Tablets, each containing 30 mg of active ingredient, are prepared as follows:

| | Quantity |
|---|---|
| Ingredient | (mg/capsule) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules, which after mixing, are compressed on a tablet machine to yield tablets each weighing 150 mg.

### Formulation Example 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, cellulose, starch, an magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount | |
|---|---|---|
| Active Ingredient | 25 | mg |
| Saturated fatty acid glycerides | to 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 ml dose are made as follows:

| Ingredient | Amount | |
|---|---|---|
| Active Ingredient | 50.0 | mg |
| Xanthan gum | 4.0 | mg |
| Sodium carboxymethyl cellulose | | |
| (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water | to 5.0 | ml |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

Hard gelatin tablets, each containing 15 mg of active ingredient are made as follows:

| | Quantity |
|---|---|
| Ingredient | (mg/capsule |
| Active Ingredient | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 560 mg quantities.

### Formulation Example 9

An intravenous formulation may be prepared as follows:

| Ingredient | Quantity | |
|---|---|---|
| Active Ingredient | 250.0 | mg |
| Isotonic saline | 1000 | ml |

Therapeutic compound compositions generally are placed into a container having a sterile access port, for example, an intravenous solution bag or vial having a stopper pierceable by a hypodermic injection needle or similar sharp instrument.

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### Formulation Example 11

An aerosol formulation may be prepared as follows:

A solution of the candidate compound in 0.5% sodium bicarbonate/saline (w/v) at a concentration of 30.0 mg/mL is prepared using the following procedure:

A. Preparation of 0.5% Sodium Bicarbonate/
Saline Stock Solution: 100.0 mL

| Ingredient | Gram/100.0 mL | Final Concentration |
|---|---|---|
| Sodium Bicarbonate | 0.5 g | 0.5% |
| Saline | q.s. ad 100.0 mL | q.s. ad 100% |

Procedure:
1. Add 0.5 g sodium bicarbonate into a 100 mL volumetric flask.
2. Add approximately 90.0 mL saline and sonicate until dissolved.
3. Q.S. to 100.0 mL with saline and mix thoroughly.

B. Preparation of 30.0 mg/mL Candidate Compound: 10.0 mL

| Ingredient | Gram/10.0 mL | Final Concentration |
|---|---|---|
| Candidate | 0.300 g | 30.0 mg/mL |
| Compound | | |
| 0.5% Sodium | q.s. ad 10.0 mL | q.s ad 100% |
| Bicarbonate/Saline | | |
| Stock Solution | | |

Procedure:
1. Add 0.300 g of the candidate compound into a 10.0 mL volumetric
flask.
2. Add approximately 9.7 mL of 0.5% sodium bicarbonate/saline stock
solution.
3. Sonicate until the candidate compound is completely dissolved.
4. Q.S. to 10.0 mL with 0.5% sodium bicarbonate/saline stock solution
and mix thoroughly.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety for or all purposes. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference in its entirety for all purposes.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

According to one aspect of the invention, the compound may be administered alone, as a combination of compounds, or in combination with anti-alpha-4-antibodies. The compounds of the present invention may also be administered in combination with an immunosuppressant, wherein the immunosuppressant is not a steroid, an anti-TNF composition, a 5-ASA composition, and combinations thereof, wherein the immunosuppressant, anti-TNF composition, and 5-ASA composition are typically used to treat the condition or disease for which the compound of the present invention is being administed. The immunosuppressant may be azathioprine, 6-mercaptopurine, methotrexate, or mycophenolate. The anti-TNF composition may be infliximab. The 5-ASA agent may be mesalazine or osalazine.

When administered in combination, the small compounds may be administered in the same formulation as these other compounds or compositions, or in a separate formulation. When administered in combinations, the steroid sparing agents may be administered prior to, following, or concurrently with the other compounds and compositions.

Pharmaceutical compositions of the invention are suitable for use in a variety of drug delivery systems. Suitable formulations for use in the present invention are found in REMINGTON'S PHARMACEUTICAL SCIENCES, Mace Publishing Company, Philadelphia, Pa., 17th ed. (1985).

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka et al., U.S. Pat. Nos. 4,235,871, 4,501,728 and 4,837,028 each of which is incorporated herein by reference in its entirety for all purposes.

Polymer Conjugates

Compounds of the present invention may be formulated and administered as polymer conjugates, preferably PEG derivatives. Polymer conjugates may exhibit benefits over non-conjugated polymers, such as improved solubility and stability.

As such, single polymer molecules may be employed for conjugation with the compounds of the present invention, although it is also contemplated that more than one polymer molecule can be attached as well. The conjugated compounds of the present invention may find utility in both in vivo as well as non-in vivo applications. Additionally, it will be recognized that the conjugating polymer may utilize any other groups, moieties, or other conjugated species, as appropriate to the end use application. By way of example, it may be useful in some applications to covalently bond to the polymer a functional moiety imparting IV-degradation resistance, or antioxidation, or other properties or characteristics to the polymer. As a further example, it may be advantageous in some applications to functionalize the polymer to render it reactive and enable it to cross-link to a drug molecule and to enhance various properties or characteristics of the overall conjugated material. Accordingly, the polymer may contain any functionality, repeating groups, linkages, or other constitutent structures which do not preclude the efficacy of the conjugated the compounds of the present invention composition for its intended purpose.

Illustrative polymers that may usefully be employed to achieve these desirable characteristics are described supra, as well as in PCT WO 01/54690 (to Zheng et al.) incorporated by reference herein in its entirety. The polymer may be coupled to the compounds of the present invention (preferably via a linker moiety) to form stable bonds that are not significantly cleavable by human enzymes. Generally, for a bond to be not “significantly” cleavable requires that no more than about 20% of the bonds connecting the polymer and the compounds of the present invention to which the polymer is linked, are cleaved within a 24 hour period, as measured by standard techniques in the art including, but not limited to, high pressure liquid chromatography (HPLC).

The compounds of the present inventions are conjugated most preferably via a terminal reactive group on the polymer although conjugations can also be branched from non-terminal reactive groups. The polymer with the reactive group(s) is designated herein as “activated polymer”. The reactive group selectively reacts with reactive groups on the compounds of the present invention. The activated polymer(s) is reacted so that attachment may occur at any available functional group on compounds of the present invention. Amino, carbon, free carboxylic groups, suitably activated carbonyl groups, hydroxyl, guanidyl, oxidized carbohydrate moieties, amino, carbon and mercapto groups of the compounds of the present invention (if available) can be used as attachment sites.

Generally, about 1.0 to about 10 moles of activated polymer per mole of the compounds of the present invention, depending on concentration, is employed. The final amount is a balance between maximizing the extent of the reaction while minimizing non-specific modifications of the product and, at the same time, defining chemistries that will maintain optimum activity, while at the same time optimizing the half-life of the compounds of the present invention. Preferably, at least about 50% of the biological activity of the compounds of the present invention is retained, and most preferably 100% is retained.

The reactions may take place by any suitable art-recognized method used for reacting biologically active materials with inert polymers. Generally, the process involves preparing an activated polymer and thereafter reacting the compounds of the present invention with the activated polymer to produce a soluble compound suitable for formulation. This modification reaction can be performed by several methods, which may involve one or more steps. The polymeric substances included herein are preferably water-soluble at room temperature. A non-limiting list of such polymers includes polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In the preferred practice of the present invention, polyalkylene glycol residues of C₁-C₄ alkyl polyalkylene glycols, preferably polyethylene glycol (PEG), or poly(oxy)alkylene glycol residues of such glycols are advantageously incorporated in the polymer systems of interest. Thus, the polymer to which the compounds of the present invention are attached may be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, polyoxyethylenated glycols, copolymers thereof and block copolymers thereof, provided that the water solubility of the block copolymer is maintained.

Examples of polyoxyethylated polyols include, but are not limited to, polyoxyethylated glycerol, polyoxyethylated sorbitol, polyoxyethylated glucose, or the like. The glycerol backbone of polyoxyethylated glycerol is the same backbone occurring naturally in, for example, animals and humans in mono-, di-, and triglycerides. Therefore, this branching would not necessarily be seen as a foreign agent in the body.

Those of ordinary skill in the art will recognize that the foregoing list is merely illustrative and that all polymer materials having the qualities described herein are contemplated. The polymer need not have any particular molecular weight, but it is preferred that the molecular weight be between about 300 and 100,000, more preferably between 10,000 and 40,000. In particular, sizes of 20,000 or more are most effective at preventing loss of the product due to filtration in the kidneys.

Polyethylene glycol (PEG) and related polyalkylene oxides (PAOs) are known in the art as being useful adjuncts for the preparation of drugs. See for example, PCT WO 93/24476. PEG has also been conjugated to proteins, peptides and enzymes to increase aqueous solubility and circulating life in vivo as well as reduce antigenicity. See, for example, U.S. Pat. Nos. 5,298,643 and 5,321,095, both to Greenwald et al. PCT WO 93/24476 discloses using an ester linkage to covalently bind an organic molecule to water-soluble polyethylene glycols. Thus, the compounds of the invention are preferably administered as polyethylene glycol (PEG) derivatives. Further description of polyethylene glycol derivatives of the compounds of the present invention and reaction conditions for preparing these derivatives are described in U.S. Ser. No. 60/538,573, entitled “Polyethylene Glycol Conjugates of Dipeptides,” filed Jan. 23, 2004, herein incorporated by reference in its entirety.

As such, the compounds or conjugates of this invention may contain one or more polyethylene glycol (PEG) substituents covalently attached thereto. Such conjugates demonstrate improved serum half-life, as compared to compounds lacking polyethylene glycol substituents. Without being limited to any theory, the improved serum half-life is believed to be associated with the covalent conjugation of at least one polyethylene glycol entity onto the structure of the compound.

The term “PEG” refers to polymers comprising multiple oxyalkylene units. Such polymers are optionally mono-capped with a substituent preferably selected from alkyl, aryl, substituted alkyl, and substituted aryl. Inclusive of such polymers are those diamino capped polyoxyalkylene polymers which are known in the art as Jeffamines®. Still further, such polymers can optionally contain one or more non-oxyalkylene units such as the commercially available poly[di(ethylene glycol)adipates, poly[di(ethylene glycol)phthalate diols, and the like.

By PEG derivative is meant a polyethylene glycol polymer in which one or both of the terminal hydroxyl groups found in polyethylene glycol itself has been modified. Examples of suitable modifications include replacing one or both hydroxyl group(s) with alternative functional groups, which may be protected or unprotected, with low molecular weight ligands, or with another macromolecule or polymer. Modification of the terminal hydroxyl groups in the polyethylene glycol may be achieved by reacting the polyethylene glycol with compounds comprising complementary reactive functional groups, including functional groups which are able to undergo a reaction with the hydroxyl groups in polyethylene glycol. The PEG derivatives of the compounds of this invention may contain one or more polyethylene glycol (PEG) substituents covalently attached thereto by a linking group.

“Linking group” or “linker” refers to a group or groups that covalently links a non-PEG substituted compound of the present invention with one or more PEG groups. Each linker may be chiral or achiral, linear, branched or cyclic and may be homogenous or heterogeneous in its atom content (e.g., linkers containing only carbon atoms or linkers containing carbon atoms as well as one or more heteroatoms present on the linker.

The PEG group or groups are covalently attached to the linker using conventional chemical techniques providing for covalent linkage of the PEG group to the linker. The linker, in turn, may be covalently attached to the otherwise, non-PEG substituted compounds of the present invention. Reaction chemistries resulting in such linkages are well known in the art. Such reaction chemistries involve the use of complementary functional groups on the linker, the non-PEG substituted compound of the present invention and the PEG groups. Preferably, the complementary functional groups on the linker are selected relative to the functional groups available on the PEG group for bonding or which can be introduced onto the PEG group for bonding. Again, such complementary functional groups are well known in the art.

Such polymers have a number average molecular weight of from about 100 to 100,000; preferably from about 1,000 to 50,000; more preferably from about 10,000 to about 40,000.

The polymer conjugates of the invention may provide enhanced in vivo retention as compared to the non-conjugated compounds. The improved retention of the conjugate within the body results in lower required dosages of the drug, which in turn results in fewer side effects and reduced likelihood of toxicity. In addition, the drug formulation comprising these polymer conjugates may be administered less frequently to the patient while achieving a similar or improved therapeutic effect. The conjugates of this invention have improved inhibition, in vivo, of adhesion of leukocytes to endothelial cells mediated by VLA-4 by competitive binding to VLA-4. Preferably, the compounds of this invention can be used in I.V. formulations.

The therapeutic dosage of the polymer conjugates of the present invention will vary according to, for example, the particular use for which the treatment is made, the manner of administration of the compound, the health and condition of the patient, and the judgment of the prescribing physician. For example, for intravenous administration, the dose will typically be in the range of about 20 μg to about 2000 μg per kilogram body weight, preferably about 20 μg to about 500 μg, more preferably about 100 μg to about 300 μg per kilogram body weight. Suitable dosage ranges for intranasal administration are generally about 0.1 pg to 1 mg per kilogram body weight. Effective doses can be extrapolated from dose-response curves derived from in vitro or animal model test systems.

When formulated and administered as polymer conjugates, the compounds or conjugates of this invention are characterized as containing one or more polyethylene glycol substituents covalently attached thereto. Without being limited to any theory, the improved serum half-life is believed to be associated with covalent conjugation of at least one polyethylene glycol entity onto the structure of the compound.

Accordingly, the compounds of the present invention may be PEG derivatives of formula XXII below: 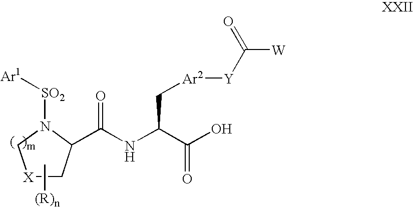
wherein:
R is selected from the group consisting of a PEG moiety, amino, substituted amino, alkyl and substituted alkyl wherein each amino, substituted amino, alkyl and substituted alkyl is optionally substituted with a PEG moiety wherein, in each case, the PEG moiety optionally comprises a linker which covalently links the PEG moiety;
Ar¹ is selected from the group consisting of aryl, substituted aryl, heteroaryl and substituted heteroaryl wherein each of aryl, substituted aryl, heteroaryl and substituted heteroaryl is optionally substituted with a PEG moiety wherein the PEG moiety optionally comprises a linker which covalently links the PEG moiety to Ar¹;
Ar² is selected from the group consisting of aryl, substituted aryl, heteroaryl and substituted heteroaryl wherein each of aryl, substituted aryl, heteroaryl and substituted heteroaryl is optionally substituted with a PEG moiety wherein the PEG moiety optionally comprises a linker which covalently links the PEG moiety to Ar²;
X is selected from the group consisting of —S—, —SO—, —SO₂ and optionally substituted —CH₂—;
Y is selected from the group consisting of —O— and —NR¹— wherein R¹ is selected from the group consisting of hydrogen and alkyl;
W is selected from the group consisting of a PEG moiety which optionally comprises a linker and —NR²R³ wherein R² and R³ are independently selected from the group consisting of alkyl, substituted alkyl, and where R² and R³, together with the nitrogen atom bound thereto, form a heterocyclic ring or a substituted heterocyclic ring wherein each of alkyl, substituted alkyl, heterocyclic and substituted heterocyclic is optionally substituted with a PEG moiety which further optionally comprises a linker;
m is an integer equal to 0, 1 or 2;
n is an integer equal to 0 to 2; and
pharmaceutically acceptable salts thereof;
provided that at least one of R, Ar¹, Ar², W and —NR²R³ contains a PEG moiety;
further provided that when R is a PEG moiety, n is one and X is not —S—, —SO—, or —SO₂—;
and still further provided that the compound of formula XXII has a molecular weight of no more than 100,000.

Preferably the PEG derivates of formula XXII are the of the L isomer as shown below:

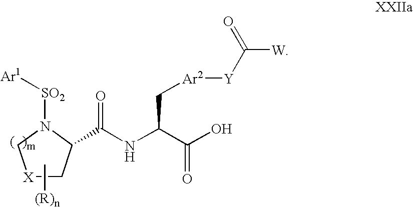

In another aspect, the compounds of the present invention may be PEG derivatives of formula XXIII below: 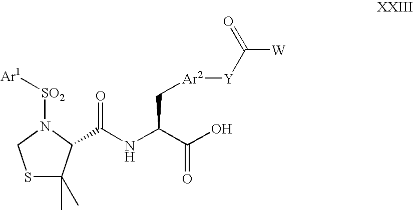
wherein:
Ar¹, Ar², Y and W are as defined above; and
pharmaceutically acceptable salts thereof;
provided that at least one of Ar¹, Ar², W and —NR²R³ contains a PEG moiety which optionally comprises a linker;
and further provided that the compound of formula XXIII has a molecular weight of no more than 100,000.

In another aspect, the compounds of the present invention may be PEG derivatives of formula XXIV below: 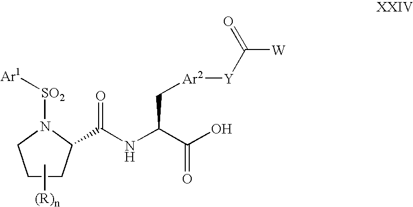
wherein:
R, Ar¹, Ar², Y, W and n are as defined above; and
pharmaceutically acceptable salts thereof;
provided that at least one of R, Ar¹, Ar², W and —NR²R³ contains a PEG moiety which optionally comprises a linker;
and further provided that the compound of formula XXVI has a molecular weight of no more than 100,000.

In another aspect, the compounds of the present invention may be PEG derivatives of formula XXV below: 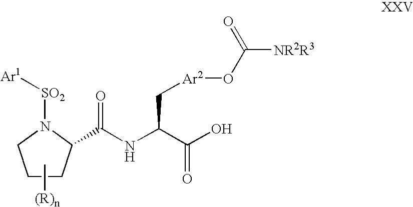
wherein:
R, R², R¹, Ar¹, Ar² and n are as defined above; and
pharmaceutically acceptable salts thereof;
provided that at least one of R, Ar¹, Ar², and —NR²R³ contains a PEG moiety which optionally comprises a linker;
and further provided that the compound of formula XXV has a molecular weight of no more than 100,000.

In another of its aspects, the compound of this invention is directed to a PEG derivative of formula XXVI below: 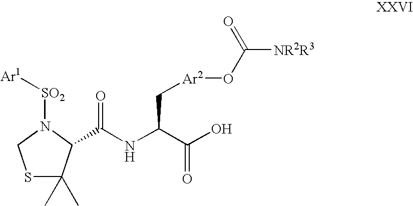
wherein:
R², R³, Ar¹, and Ar² are as defined above; and
pharmaceutically acceptable salts thereof;
provided that at least one of Ar¹, Ar² and —NR²R³ contains a PEG moiety which optionally comprises a linker;
and further provided that the compound of formula XXVI has a molecular weight of no more than 100,000.

In another aspect, the compounds of this invention can be PEG derivatives of formula XXVII: 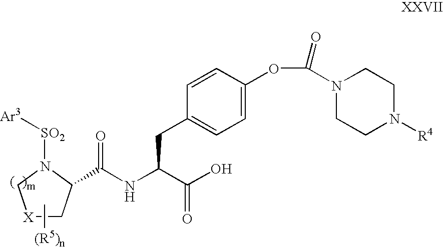
wherein:
R⁴ is a PEG moiety which optionally comprises a linker;
R⁵ is selected from the group consisting of alkyl and substituted alkyl;
Ar³ is selected from the group consisting of aryl, substituted aryl, heteroaryl and substituted heteroaryl;
X is selected from the group consisting of —S—, —SO—, and —SO₂— or optionally substituted —CH₂—;
m is an integer equal to 0, 1 or 2;
n is an integer equal to 0 to 2; and
pharmaceutically acceptable salts thereof;
provided that the compound of formula XXVII has a molecular weight of no more than 100,000.

In another aspect, the compound of the invention can be a PEG derivative of formula XXVIII: 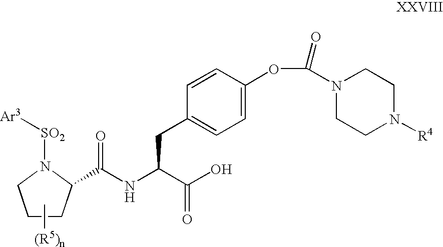
wherein:
R⁴ is a PEG moiety which optionally comprises a linker;
R⁵ is selected from the group consisting of alkyl and substituted alkyl;
Ar³ is selected from the group consisting of aryl, substituted aryl, heteroaryl and substituted heteroaryl;
n is an integer equal to 0 to 2; and
pharmaceutically acceptable salts thereof;
provided that the compound of formula XXVIII has a molecular weight of no more than 100,000.

In another aspect, the compound of the invention can be a PEG derivative of formula XXIX: 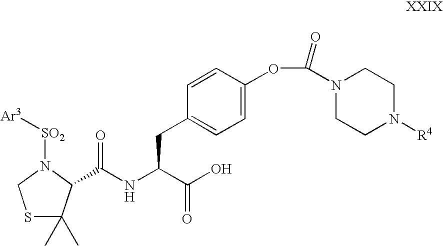
wherein:
R⁴ is a PEG moiety which optionally comprises a linker;
Ar³ is selected from the group consisting of aryl, substituted aryl, heteroaryl and substituted heteroaryl;
pharmaceutically acceptable salts thereof;
provided that the compound of formula XXIX has a molecular weight of no more than 100,000.

Preferably, when Ar¹ does not contain a PEG moiety, Ar¹ in formulas XXII-XXVI and Ar³ in formulas XXVII-XXIX is selected from the group consisting of:
phenyl,
4-methylphenyl,
4-t-butylphenyl,
2,4,6-trimethylphenyl,
2-fluorophenyl,
3-fluorophenyl,
4-fluorophenyl,
2,4-difluorophenyl,
3,4-difluorophenyl,
3,5-difluorophenyl,
2-chlorophenyl,
3-chlorophenyl,
4-chlorophenyl,
3,4-dichlorophenyl,
3,5-dichlorophenyl,
3-chloro-4-fluorophenyl,
4-bromophenyl,
2-methoxyphenyl,
3-methoxyphenyl,
4-methoxyphenyl,
3,4-dimethoxyphenyl,
4-t-butoxyphenyl,
4-(3′-dimethylamino-n-propoxy)-phenyl,
2-carboxyphenyl,
2-(methoxycarbonyl)phenyl,
4-(H₂NC(O)-)phenyl,
4-(H₂NC(S)-)phenyl,
4-cyanophenyl,
4-trifluoromethylphenyl,
4-trifluoromethoxyphenyl,
3,5-di-(trifluoromethyl)phenyl,
4-nitrophenyl,
4-aminophenyl,
4-(CH₃C(O)NH-)phenyl,
4-(PhNHC(O)NH-)phenyl,
4-amidinophenyl,
4-methylamidinophenyl,
4-[CH₃SC(═NH)-]phenyl,
4-chloro-3-[H₂NS(O)₂-]phenyl,
1-naphthyl,
2-naphthyl,
pyridin-2-yl,
pyridin-3-yl,
pyridine-4-yl,
pyrimidin-2-yl,
quinolin-8-yl,
2-(trifluoroacetyl)-1,2,3,4-tetrahydroisoquinolin-7-yl,
2-thienyl,
5-chloro-2-thienyl,
2,5-dichloro-4-thienyl,
1-N-methylimidazol-4-yl,
1-N-methylpyrazol-3-yl,
1-N-methylpyrazol-4-yl,
1-N-butylpyrazol-4-yl,
1-N-methyl-3-methyl-5-chloropyrazol-4-yl,
1-N-methyl-5-methyl-3-chloropyrazol-4-yl,
2-thiazolyl, and
5-methyl-1,3,4-thiadiazol-2-yl.

When Ar¹ contains a PEG group, Ar¹ is preferably of the formula:
—Ar¹-Z-(CH₂CHR⁷O)ₚR⁸
wherein:
Ar¹ is selected from the group consisting of aryl, substituted aryl, heteroaryl, and substituted heteroaryl,
Z is selected from the group consisting of a covalent bond, a linking group of from 1 to 40 atoms, —O—, and —NR⁹—, where R⁹ is selected from the group consisting of hydrogen and alkyl,
R⁷ is selected from the group consisting of hydrogen and methyl;
R⁸ is selected from the group consisting of hydrogen, alkyl, substituted alkyl, aryl, substituted aryl, and —CH₂CHR⁷NR¹⁰R¹¹ where R⁷ is as defined above and R¹⁰ and R¹¹ are independently selected from the group consisting of hydrogen and alkyl; and
p is an integer such that the molecular weight of the PEG moiety ranges from 100 to 100,000.

Preferably, when R does not contain a PEG moiety, the substituent of the formula: 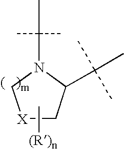
where X, m and n are as defined above, and R¹ is alkyl or substituted alkyl is preferably selected from the group consisting of:
azetidinyl, thiazolidinyl, piperidinyl, piperazinyl, thiomorpholinyl, pyrrolidinyl, 4-hydroxypyrrolidinyl, 4-oxopyrrolidinyl, 4-fluoropyrrolidinyl, 4,4-difluoropyrrolidinyl, 4-(thiomorpholin-4-ylC(O)O-)pyrrolidinyl, 4-[CH₃S(O)₂O-]pyrrolidinyl, 3-phenylpyrrolidinyl, 3-thiophenylpyrrolidinyl, 4-aminopyrrolidinyl, 3-methoxypyrrolidinyl, 4,4-dimethylpyrrolidinyl, 4-N-Cbz-piperazinyl, 4-[CH₃S(O)₂-]piperazinyl, 5,5-dimethylthiazolindin-4-yl, 1,1-dioxo-thiazolidinyl, 1,1-dioxo-5,5-dimethylthiazolidin-2-yl and 1,1-dioxothiomorpholinyl.

When the substituent of the formula: 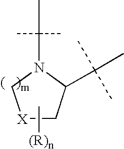
contains a PEG moiety, then preferably the substituent is of the formula: 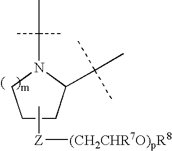
   wherein:
m is an integer equal to zero, one or two;
Z is selected from the group consisting of a covalent bond, a linking group of from 1 to 40 atoms, —O—, and —NR⁹—, where R⁹ is selected from the group consisting of hydrogen and alkyl,
R⁷ is selected from the group consisting of hydrogen and methyl;
R⁸ is selected from the group consisting of hydrogen, alkyl, substituted alkyl, aryl, substituted aryl, and —CH₂CHR⁷NR¹⁰R¹¹ where R⁷ is as defined above and R¹⁰ and R¹¹ are independently selected from the group consisting of hydrogen and alkyl; and
p is an integer such that the molecular weight of the PEG moiety ranges from 100 to 100,000.

Preferably, when Ar² does not contain a PEG moiety, Ar² in formulas I-V is preferably selected from the group consisting of phenyl, 2-pyridyl, 3-pyridyl, 4-pyridyl, and 4-pyrid-2-onyl.

When Ar² contains a PEG moiety, Ar² in formulas XXII-XXVI is preferably represented by the formula: 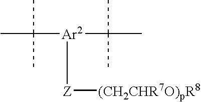
where Ar² is selected from the group consisting of aryl, substituted aryl, heteroaryl and substituted heteroaryl;
Z is selected from the group consisting of a covalent bond, a linking group of from 1 to 40 atoms, —O—, and —NR⁹—, where R⁹ is selected from the group consisting of hydrogen and alkyl,
R⁷ is selected from the group consisting of hydrogen and methyl;
R⁸ is selected from the group consisting of hydrogen, alkyl, substituted alkyl, aryl, substituted aryl, and —CH₂CHR⁷NR¹⁰R¹¹ where R⁷ is as defined above and R¹⁰ and R¹¹ are independently selected from the group consisting of hydrogen and alkyl; and
p is an integer such that the molecular weight of the PEG moiety ranges from 100 to 100,000.

Preferably, in formulas XXII-XXIV, —YC(O)W is —OC(O)NR²R³. When R² and R³ do not contain a PEG moiety, —OC(O)NR²R³ in formulas XXII-XXVI is preferably selected from the group:
(CH₃)₂NC(O)O—,
(piperidin-1-yl)C(O)O—,
(4-hydroxypiperidin-1-yl)C(O)O—,
(4-formyloxypiperidin-1-yl)C(O)O—,
(4-ethoxycarbonylpiperidin-1-yl)C(O)O—,
(4-carboxylpiperidin-1-yl)C(O)O—,
(3-hydroxymethylpiperidin-1-yl)C(O)O—,
(4-hydroxymethylpiperidin-1-yl)C(O)O—,
(4-piperidon-1-yl ethylene ketal)C(O)O—,
(piperazin-1-yl)-C(O)O—,
(1-Boc-piperazin-4-yl)-C(O)O—,
(4-methylpiperazin-1-yl)C(O)O—,
(4-methylhomopiperazin-1-yl)C(O)O—,
(4-(2-hydroxyethyl)piperazin-1-yl)C(O)O—,
(4-phenylpiperazin-1-yl)C(O)O—,
(4-(pyridin-2-yl)piperazin-1]-yl)C(O)O—,
(4-(4-trifluoromethylpyridin-2-yl)piperazin-1-yl)C(O)O—,
(4-(pyrimidin-2-yl)piperazin-1-yl)C(O)O—,
(4-acetylpiperazin-1-yl)C(O)O—,
(4-(phenylC(O)-)piperazin-1-yl)C(O)O—,
(4-(pyridin-4′-ylC(O)-)piperazin-1-yl)C(O)O,
(4-(phenylNHC(O)-)piperazin-1-yl)C(O)O—,
(4-(phenylNHC(S)-)piperazin-1-yl)C(O)O—,
(4-methanesulfonylpiperazin-1-yl-C(O)O—,
(4-trifluoromethanesulfonylpiperazin-1-yl-C(O)O—,
(morpholin-4-yl)C(O)O—,
(thiomorpholin-4-yl)C(O)O—,
(thiomorpholin-4′-yl sulfone)-C(O)O—,
(pyrrolidin-1-yl)C(O)O—,
(2-methylpyrrolidin-1-yl)C(O)O—,
(2-(methoxycarbonyl)pyrrolidin-1-yl)C(O)O—,
(2-(hydroxymethyl)pyrrolidin-1-yl)C(O)O—,
(2-(N,N-dimethylamino)ethyl)(CH₃)NC(O)O—,
(2-(N-methyl-N-toluene-4-sulfonylamino)ethyl)(CH₃)N—C(O)O—,
(2-(morpholin-4-yl)ethyl)(CH₃)NC(O)O—,
(2-(hydroxy)ethyl)(CH₃)NC(O)O—,
bis(2-(hydroxy)ethyl)NC(O)O—,
(2-(formyloxy)ethyl)(CH₃)NC(O)O—,
(CH₃₀C(O)CH₂)HNC(O)O—, and
2-[(phenylNHC(O)O-)ethyl-]HNC(O)O—.

When R² and/or R³ comprise a PEG moiety, the PEG moiety is preferably represented by the formula:
-Z′-(CH₂CHR⁷O)ₚR⁸
Z′ is selected from the group consisting of a covalent bond and a linking group of from 1 to 40 atoms;
R⁷ is selected from the group consisting of hydrogen and methyl;
R⁸ is selected from the group consisting of hydrogen, alkyl, substituted alkyl, aryl, substituted aryl, and —CH₂CHR⁷NR¹⁰R¹¹ where R⁷ is as defined above and R¹⁰ and R¹¹ are independently selected from the group consisting of hydrogen and alkyl; and
p is an integer such that the molecular weight of the PEG moiety ranges from 100 to 100,000.

Preferred —YC(O)W substituents comprising a PEG moiety include the following:
—OC(O)NH(CH₂CH₂O)ₚCH₂CH₂NH₂;
—OC(O)NH(CH₂CH(CH₃)O)ₚCH₂CH(CH₃)NH₂;
—NHC(O)O(CH₂CH₂O)ₚH;
—NHC(O)O(CH₂CH(CH₃)O)ₚH;
—NHC(O)O(CH₂CH₂O)ₚCH₃;
—NHC(O)O(CH₂CH(CH₃)O)ₚCH₃;
—NHC(O)O(CH₂CH₂O)ₚ-φ;
—NHC(O)O(CH₂CH(CH₃)O)ₚ-φ;
—NHC(O)NH(CH₂CH₂O)ₚCH₂CH₂NH₂;
—NHC(O)NH(CH₂CH(CH₃)O)ₚCH₂CH(CH₃)NH₂;
—OC(O)NH-(1,4)-φ-O—(CH₂CH₂O)ₚH;
—OC(O)NH-(1,4)-φ-O—(CH₂CH(CH₃)O)ₚH;
—OC(O)NH-(1,4)-φ-O—(CH₂CH₂O)ₚCH₃;
—OC(O)NH-(1,4)-φ-O—(CH₂CH(CH₃)O)ₚCH₃;
—OC(O)NH(CH₂CH(CH₃)O)ₚCH₂CH(CH₃)OCH₃;
—NHC(O)NH(CH₂CH₂O)ₚCH₃;
—NHC(O)NH(CH₂CH(CH₃)O)ₚCH₃; 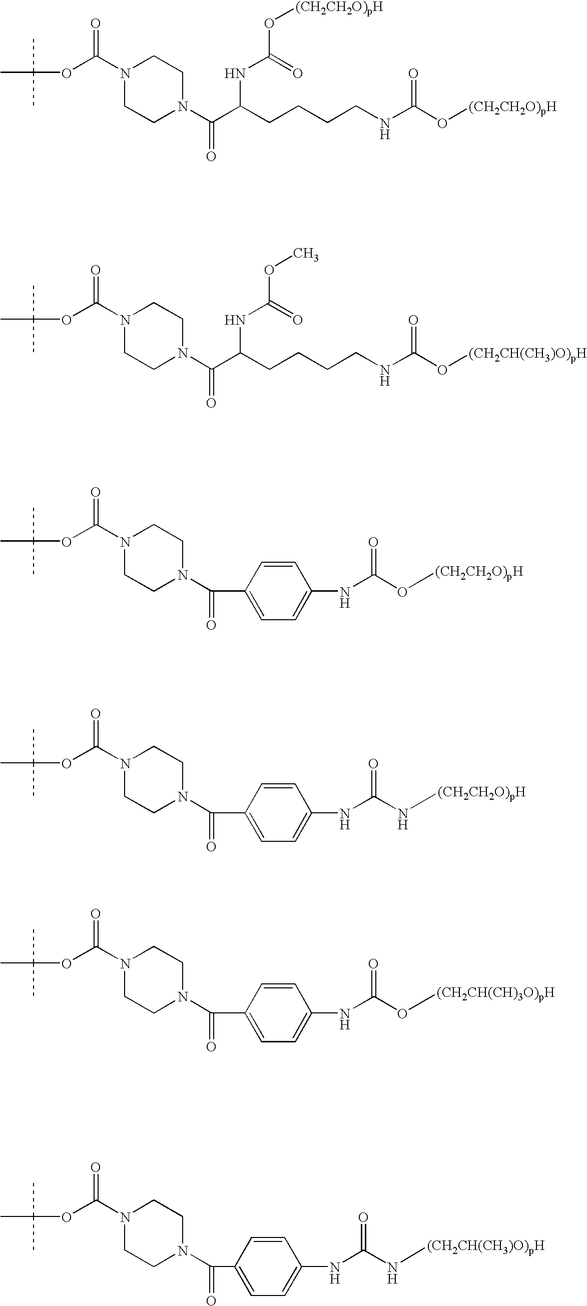 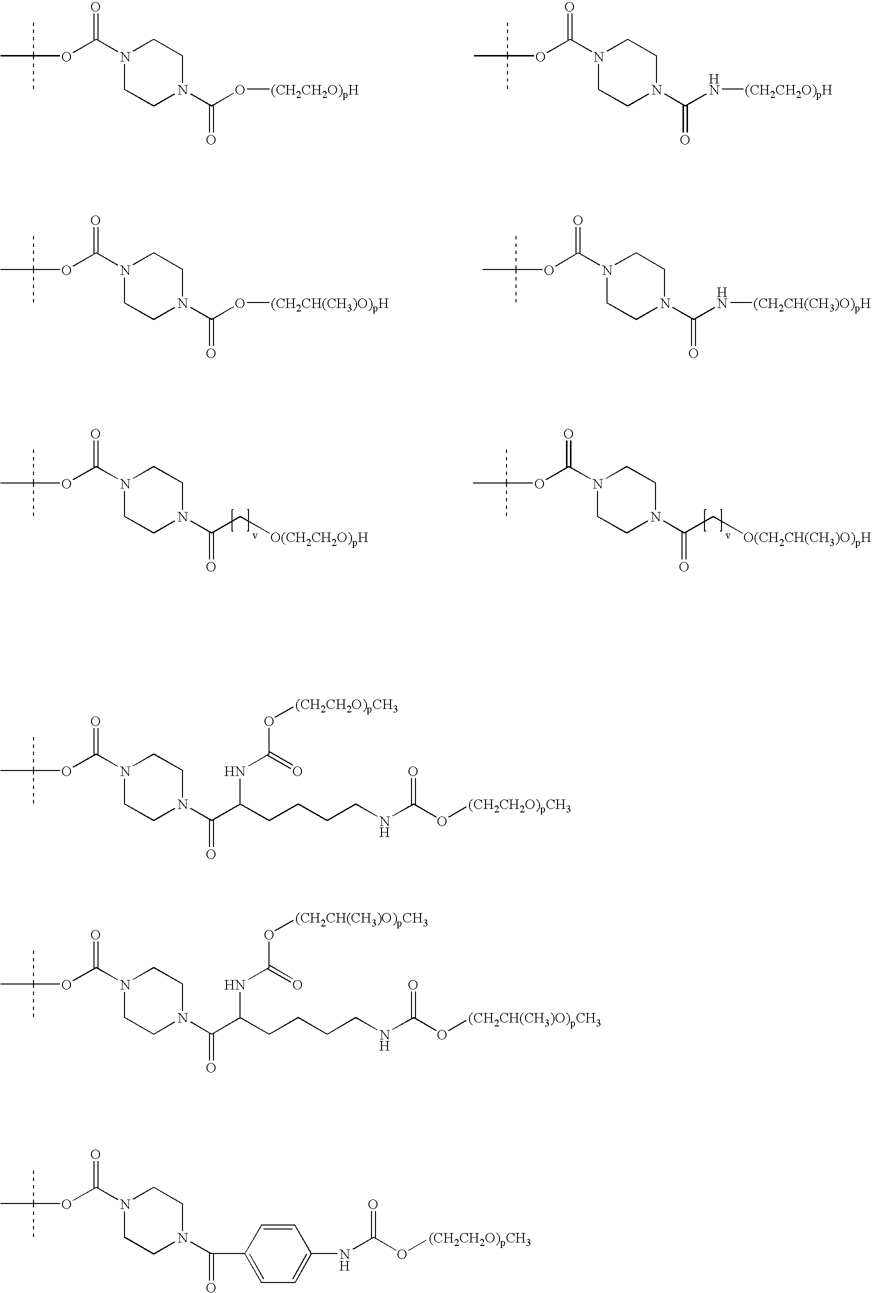 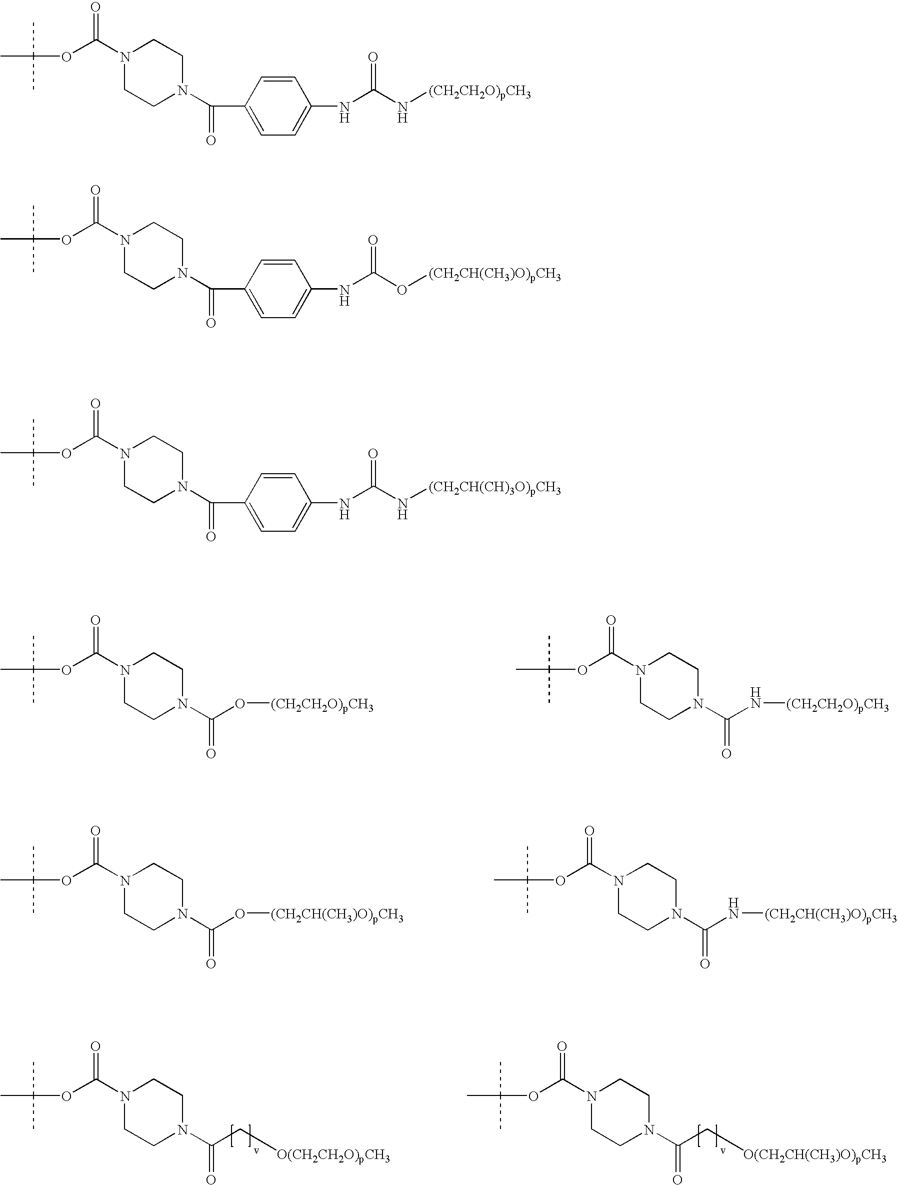 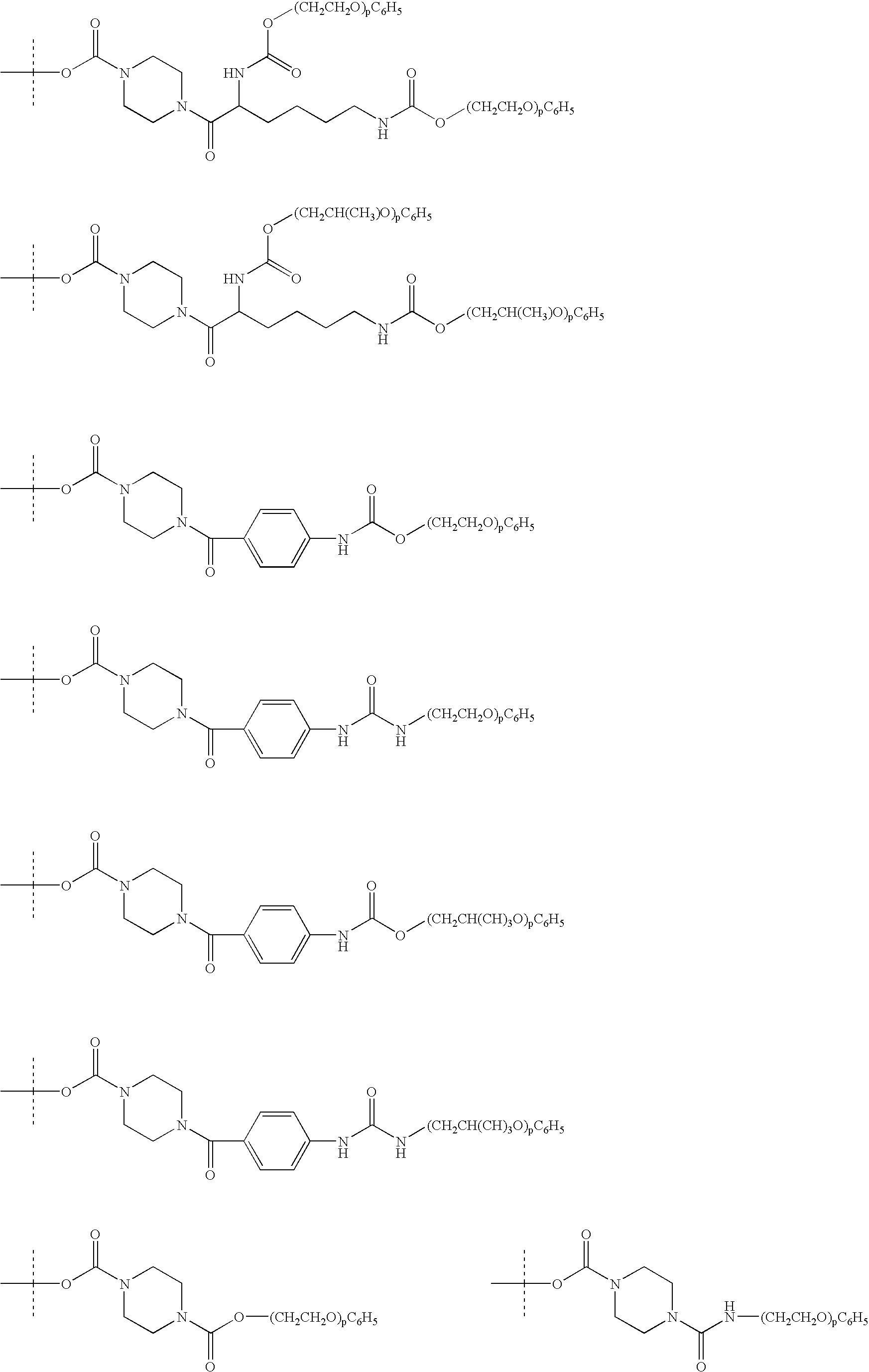 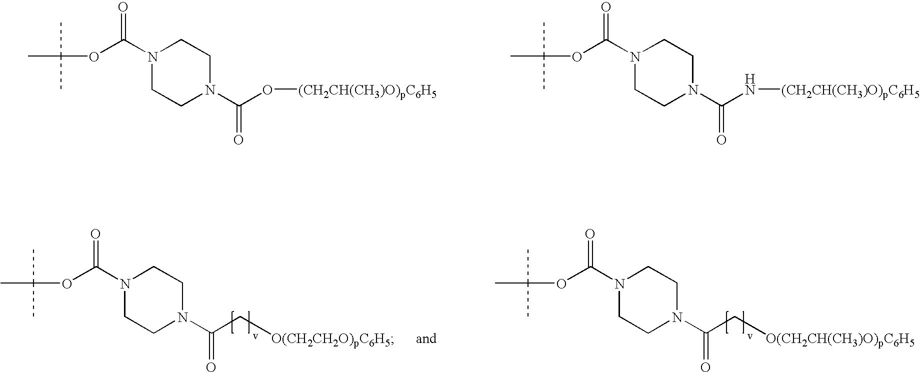
where φ or C₆H₅ is phenyl and p is an integer such that the molecular weight of the PEG moiety ranges from about 100 to 100,000 and v is 1 to 5.

Preferred PEG derivatives of this invention include those set forth below: 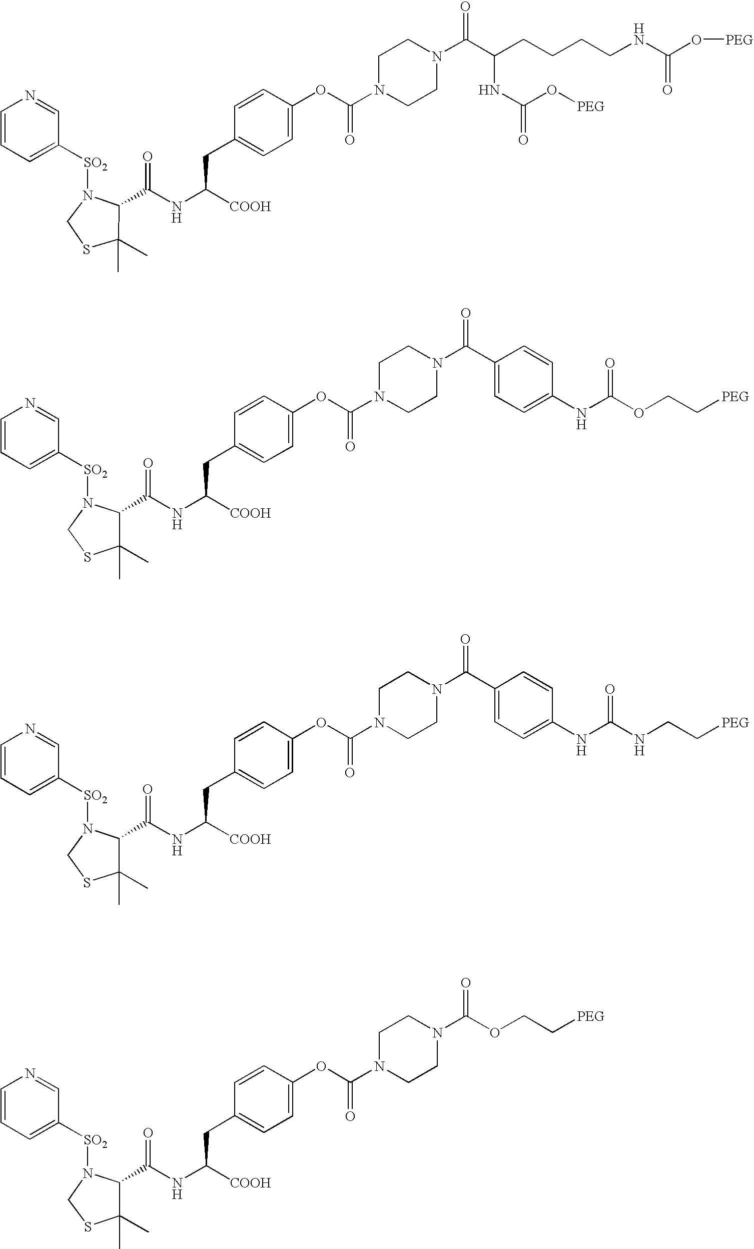 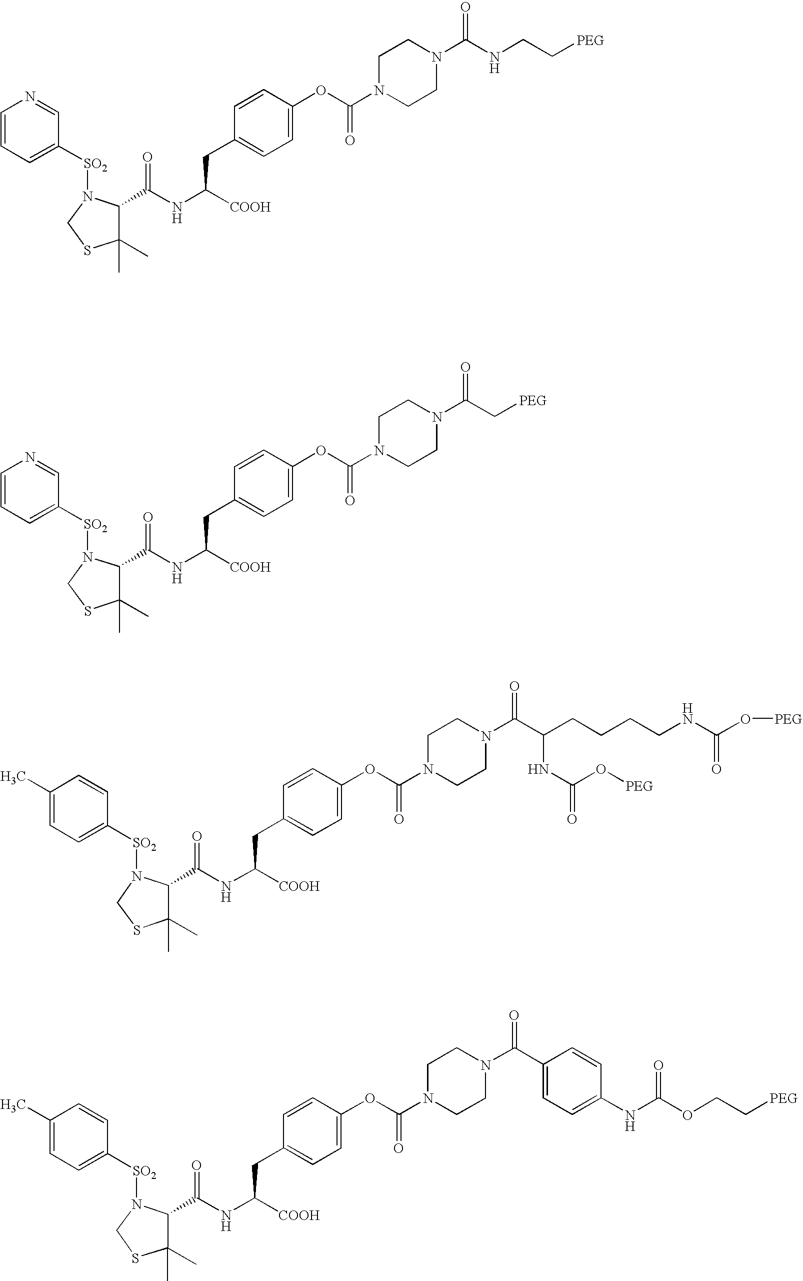 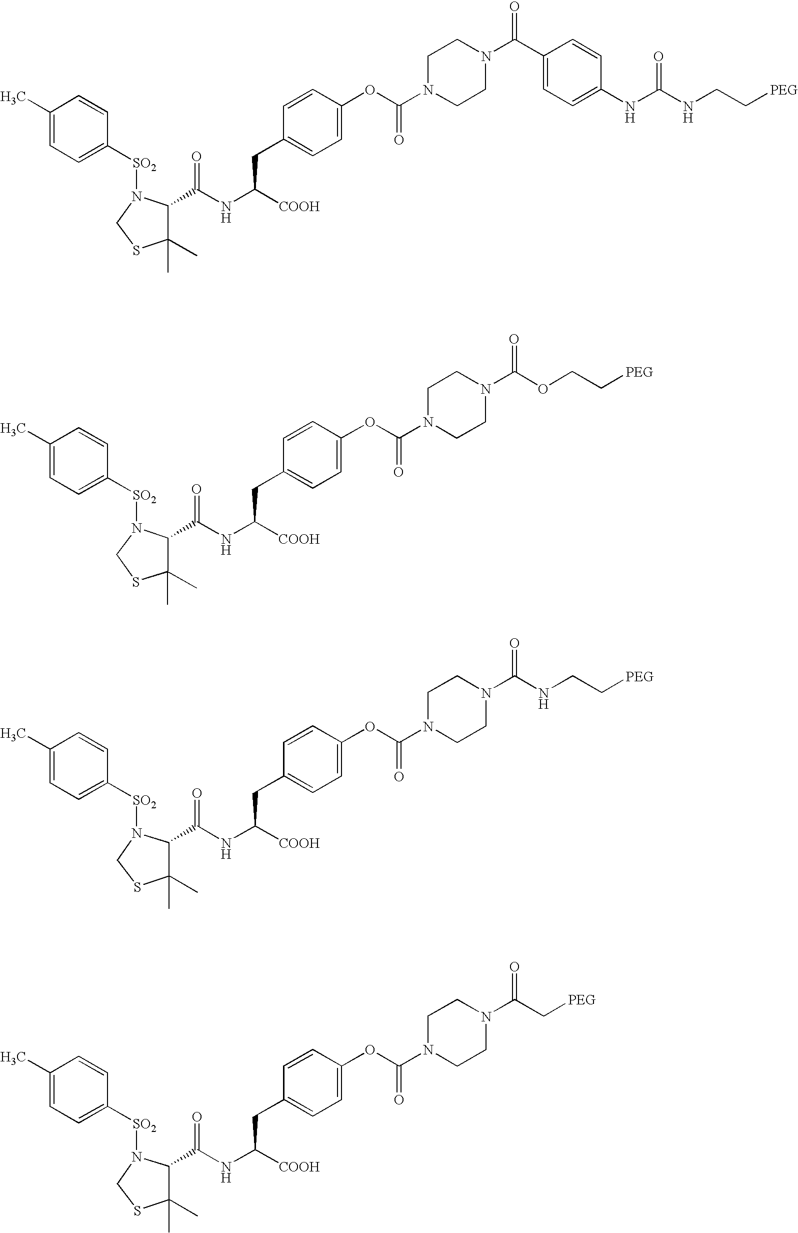
where, in each case, PEG is a methyl capped polyoxyethylene group having a molecular weight (Mw) of approximately 20,000.

“Linking group” or “linker” of from 1 to 40 atoms is a di- to hexavalent group or groups that covalently links a non-PEG substituted compound of formula I (i.e., none of Ar¹, Ar², R or —Y—C(O)—W— contain a PEG group) with 1 to 5 PEG groups. Each linker may be chiral or achiral, linear, branched or cyclic and may be homogenous or heterogeneous in its atom content (e.g., linkers containing only carbon atoms or linkers containing carbon atoms as well as one or more heteroatoms present on the linker in the form of alcohols, ketones, aldehydes, carboxyl groups, amines, amides, carbamates, ureas, thiols, ethers, etc., or residues thereof) Preferably, the linker contains 1 to 25 carbon atoms and 0 to 15 heteroatoms selected from oxygen, NR²², sulfur, —S(O)— and —S(O)₂—, where R is as defined above.

The PEG group or groups are covalently attached to the linker using conventional chemical techniques providing for covalent linkage of the PEG group to the linker. The linker, in turn, is covalently attached to the otherwise, non-PEG substituted compound of formula I. Reaction chemistries resulting in such linkages are well known in the art. Such reaction chemistries involve the use of complementary functional groups on the linker, the non-PEG substituted compound of formula XXII and the PEG groups. Preferably, the complementary functional groups on the linker are selected relative to the functional groups available on the PEG group for bonding or which can be introduced onto the PEG group for bonding. Again, such complementary functional groups are well known in the art. For example, reaction between a carboxylic acid of either the linker or the PEG group and a primary or secondary amine of the PEG group or the linker in the presence of suitable, well-known activating agents results in formation of an amide bond covalently linking the PEG group to the linker; reaction between an amine group of either the linker or the PEG group and a sulfonyl halide of the PEG group or the linker results in formation of a sulfonamide bond covalently linking the PEG group to the linker; and reaction between an alcohol or phenol group of either the linker or the PEG group and an alkyl or aryl halide of the PEG group or the linker results in formation of an ether bond covalently linking the PEG group to the linker.

Table 11 below illustrates numerous complementary reactive groups and the resulting bonds formed by reaction therebetween.

TABLE 11
Representative Complementary Binding Chemistries

| First Reactive Group | Second Reactive Group | Linkage |
|---|---|---|
| Hydroxyl | Isocyanate | urethane |
| Amine | Epoxide | β-hydroxyamine |
| sulfonyl halid | Amine | sulfonamide |
| Carboxyl | Amine | amide |
| Hydroxyl | alkyl/aryl halide | ether |

Preferred linkers include, by way of example, the following —O—, —NR²²—, —NR²²C(O)O—, —OC(O)NR²²—, —NR¹²C(O)—, —C(O)NR²², —NR²²C(O)NR²²—, -alkylene-NR²²C(O)O—, -alkylene-NR²C(O)NR²²—, -alkylene-OC(O)NR²²—, -alkylene-NR²²—, -alkylene-O—, -alkylene-NR²²C(O)—, -alkylene-C(O)NR²²—, —NR³C(O)O-alkylene-, —NR²²C(O)NR²²-alkylene-, —OC(O)NR²²-alkylene, —NR²²-alkylene-, —O-alkylene-, —NR²²C(O)-alkylene-, —C(O)NR²²-alkylene-, -alkylene-NR²²C(O)O-alkylene-, -alkylene-NR³C(O)NR²²-alkylene-, -alkylene-OC(O)NR²²-alkylene-, -alkylene-NR²²-alkylene-, alkylene-O-alkylene-, -alkylene-NR²C(O)-alkylene-, —C(O)NR²²-alkylene-, and 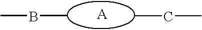
where 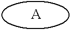
is selected from the group consisting of aryl, substituted aryl, cycloalkyl, substituted cycloalkyl, heteroaryl, substituted heteroaryl, heterocyclic and substituted heterocyclic, and B and C are independently selected from the group consisting of a bond, —O—, CO, —NR²²—, —NR²²C(O)O—, —OC(O)NR²²—, —NR²²C(O)—, —C(O)NR²²—, —NR²²C(O)NR²²—, -alkylene-NR²²C(O)O—, -alkylene-NR²²C(O)NR²²—, -alkylene-OC(O) NR²²—, -alkylene-NR²²—, -alkylene-O—, -alkylene-NR²²C(O)—, alkylene-C(O)NR²²—, —NR²²C(O)O-alkylene-, —NR²²C(O)NR²²-alkylene-, —OC(O) NR²²-alkylene-, —NR²²-alkylene-, —O-alkylene-, —NR²²C(O)-alkylene-, —C(O)NR²-alkylene-, -alkylene-NR²²C(O)O-alkylene-, -alkylene-NR²²C(O)NR²²-alkylene-, —alkylene-OC(O)NR²²-alkylene-, -alkylene-NR²²-alkylene-, alkylene-O-alkylene-, —alkylene-NR²²C(O)-alkylene-, and —C(O)NR²²-alkylene-, where R²² is as defined above.

“PEG” or “PEG moiety” refers to polymers comprising multiple oxyalkylene units. Such polymers are optionally mono-capped with a substituent preferably selected from alkyl, aryl, substituted alkyl, and substituted aryl. Inclusive of such polymers are those diamino capped polyoxyalkylene polymers which are known in the art as Jeffamines®. Still further, such polymers can optionally contain one or more non-oxyalkylene units such as the commercially available poly[di(ethylene glycol)adipates, poly[di(ethylene glycol)phthalate diols, and the like. Also included are block copolymers of oxyalkylene, polyethylene glycol, polypropylene glycol, and polyoxyethylenated polyol units.

Such polymers have a number average molecular weight of from about 100 to 100,000; preferably from about 1,000 to 50,000; more preferably from about 10,000 to about 40,000. In a particularly preferred embodiment, the molecular weight of the total amount of PEG arising from single or multiple PEG moieties bound in the molecule does not exceed 100,000; more preferably 50,000 and even more preferably 40,000.

In a preferred embodiment, the -[linking group]ᵤ-PEG group where u is zero or one can be represented by the formula:
-Z′-[(CH₂CHR⁷O)ₚR⁸]ₜ
where Z′ is selected from the group consisting of a covalent bond, a linking group of from 1 to 40 atoms, —O—, —S—, —NR²²—, —C(O)O—, —C(O)NR²²—, and —C(O)— where R²² is selected from the group consisting of hydrogen and alkyl,
R⁷ is selected from the group consisting of hydrogen and methyl;
R⁸ is selected from the group consisting of hydrogen, alkyl, substituted alkyl, aryl, substituted aryl, —CH₂CHR⁷SR⁷ and —CH₂CHR⁷NR¹⁰R¹¹ where R⁷ is as defined above and R¹⁰ and R¹¹ are independently selected from the group consisting of hydrogen and alkyl;
p is an integer such that the molecular weight of the PEG moiety ranges from 100 to 100,000; and
t is an integer from 1 to 5 provided that t is one less than the valency of the linking group and is one when there is no linking group.

When Z′ is linking group, multiple PEG groups can be present. For example, if the linking group is trivalent, then 2 PEG groups can be attached and the remaining valency is employed to link to the molecule of formula XXII. Preferably the number of PEG groups is 1 or 2. In any event, when multiple PEG groups are present, the total aggregate molecular weight of the PEG groups does not exceed 100,000.

PEG Derivative Preparation

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Second Edition, Wiley, New York, 1991, and references cited therein.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The compounds of this invention are preferably characterized by containing one or more PEG moieties at one of several sites of a compound of formula XXIIa:

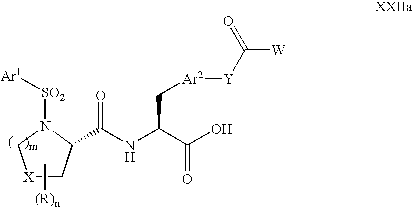

Specifically, the PEG moiety can be incorporated into the Ar¹ substituent, the R substituent, the Ar² substituent and/or in the —YC(O)W substituent wherein the PEG moiety is either directly attached or is attached via a linker. The synthetic protocol for insertion of a PEG moiety at each of these positions is similar and entails reaction of a functional group on the PEG moiety or the linking group covalently bound to the PEG moiety with a complementary functional group on the non-PEG substituted compounds of formula XXIIa.

Initially, non-PEG substituted compounds of formula XXIIa are well known in the art and are exemplified in a number of issued patents including, without limitation, U.S. Pat. Nos. 6,489,300 and 6,436,904 both of which are incorporated herein by reference in their entirety. Non-PEG variants of compounds of formula Ia include those having complementary functional groups or groups derivatizable to complementary functional groups on one or more of the Ar¹, R, Ar² and —YC(O)W moieties. For illustrative purposes, compounds having a complementary functional group (—OH) on the Ar² moiety (e.g., tyrosine) are recited below as a suitable starting point for addition of a PEG group to the molecule either directly or through a linker.

Such compounds can be prepared by first coupling a heterocyclic amino acid, 1, with an appropriate aryl sulfonyl chloride as illustrated in Scheme 1 below: 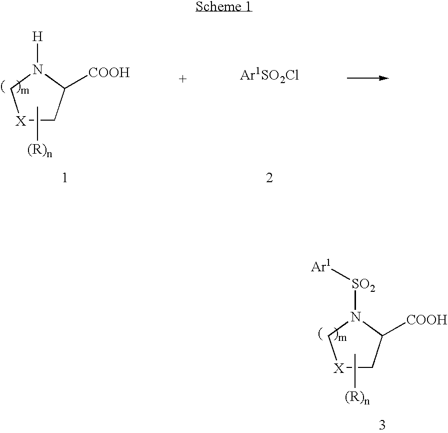
where R, Ar¹, X, m and n are as defined above.

Specifically, in Scheme 1 above, heterocyclic amino acid, 1, is combined with a stoichiometric equivalent or excess amount (preferably from about 1.1 to about 2 equivalents) of arylsulfonyl halide, 2, in a suitable inert diluent such as dichloromethane and the like. Generally, the reaction is conducted at a temperature ranging from about −70° C. to about 40° C. until the reaction is substantially complete, which typically occurs within 1 to 24 hours. Preferably, the reaction is conducted in the presence of a suitable base to scavenge the acid generated during the reaction. Suitable bases include, by way of example, tertiary amines, such as triethylamine, diisopropylethylamine, N-methyl-morpholine and the like. Alternatively, the reaction can be conducted under Schotten-Baumann-type conditions using an aqueous alkali solution such as an aqueous solution of sodium hydroxide, an aqueous phosphate solution buffered to pH 7.4, and the like. The resulting product, 3, can be recovered by conventional methods, such as chromatography, filtration, evaporation, crystallization, and the like or, alternatively, used in the next step without purification and/or isolation.

Heterocyclic amino acids, 1, employed in the above reaction are either known compounds or compounds that can be prepared from known compounds by conventional synthetic procedures. Examples of suitable amino acids for use in this reaction include, but are not limited to, L-proline, trans-4-hydroxyl-L-proline, cis-4-hydroxyl-L-proline, trans-3-phenyl-L-proline, cis-3-phenyl-L-proline, L-(2-methyl)proline, L-pipecolinic acid, L-azetidine-2-carboxylic acid, L-thiazolidine-4-carboxylic acid, L-(5,5-dimethyl)thiazolidine-4-carboxylic acid, L-thiamorpholine-3-carboxylic acid. If desired, the corresponding carboxylic acid esters of the amino acids, 1, such as the methyl esters, ethyl esters, t-butyl esters, and the like, can be employed in the above reaction with the arylsulfonyl chloride. Subsequent hydrolysis of the ester group to the carboxylic acid using conventional reagents and conditions, i.e., treatment with an alkali metal hydroxide in an inert diluent such as methano/water, then provides the N-sulfonyl amino acid, 3.

Similarly, the arylsulfonyl chlorides, 2, employed in the above reaction are either known compounds or compounds that can be prepared from known compounds by conventional synthetic procedures. Such compounds are typically prepared from the corresponding sulfonic acid, i.e., from compounds of the formula Ar¹SO₃H where Ar¹ is as defined above, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0° C. to about 80° C. for about 1 to about 48 hours to afford the sulfonyl chloride. Alternatively, the arylsulfonyl chlorides, 2, can be prepared from the corresponding thiol compound, i.e., from compounds of the Ar¹—SH where Ar¹ is as defined herein, by treating the thiol with chlorine (Cl₂) and water under conventional reaction conditions.

Alternatively, arylsulfonyl chlorides, 2, employed in the above reaction may be prepared by chlorosulfonylation of substituted benzene or heterocycloalkyl group using Cl—SO₃H.

Examples of arylsulfonyl chlorides suitable for use in this invention include, but are not limited to, benzenesulfonyl chloride, 1-naphthalenesulfonyl chloride, 2-naphthalenesulfonyl chloride, p-toluenesulfonyl chloride, o-toluenesulfonyl chloride, 4-acetamidobenzenesulfonyl chloride, 4-tert-butylbenzenesulfonyl chloride, 4-bromobenzenesulfonyl chloride, 2-carboxybenzenesulfonyl chloride, 4-cyanobenzenesulfonyl chloride, 3,4-dichlorobenzenesulfonyl chloride, 3,5-dichlorobenzenesulfonyl chloride, 3,4-dimethoxybenzenesulfonyl chloride, 3,5-ditrifluoromethylbenzenesulfonyl chloride, 4-fluorobenzenesulfonyl chloride, 4-methoxybenzenesulfonyl chloride, 2-methoxycarbonylbenzenesulfonyl chloride, 4-methylamido-benzenesulfonyl chloride, 4-nitrobenzenesulfonyl chloride, 4-trifluoromethyl-benzenesulfonyl chloride, 4-trifluoromethoxybenzenesulfonyl chloride, 2,4,6-trimethylbenzenesulfonyl chloride, 2-thiophenesulfonyl chloride, 5-chloro-2-thiophenesulfonyl chloride, 2,5-dichloro-4-thiophenesulfonyl chloride, 2-thiazolesulfonyl chloride, 2-methyl-4-thiazolesulfonyl chloride, 1-methyl-4-imidazolesulfonyl chloride, 1-methyl-4-pyrazolesulfonyl chloride, 5-chloro-1,3-dimethyl-4-pyrazolesulfonyl chloride, 3-pyridinesulfonyl chloride, 2-pyrimidinesulfonyl chloride and the like. If desired, a sulfonyl fluoride, sulfonyl bromide or sulfonic acid anhydride may be used in place of the sulfonyl chloride in the above reaction to form the N-sulfonyl amino acid, 3.

The N-arylsulfonyl amino acid, 3, is then coupled to commercially available tyrosine esters as shown in Scheme 2 below: 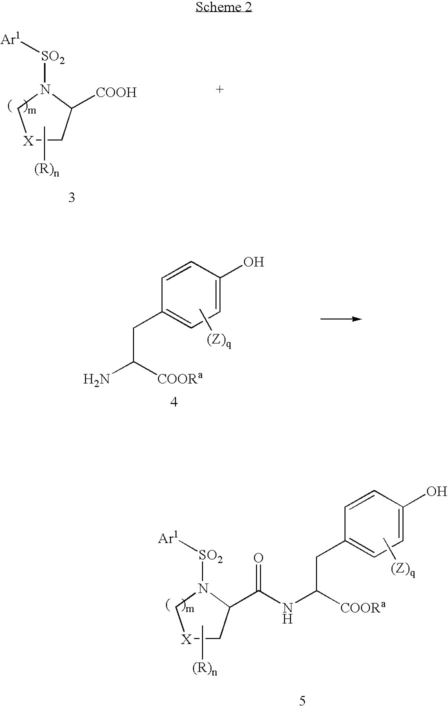
where R, Ar¹, X, m and n are as defined above, R^{a} is hydrogen or alkyl but preferably is an alkyl group such as t-butyl, Z represents optional substitution on the aryl ring and q is zero, one or two.

This coupling reaction is typically conducted using well-known coupling reagents such as carbodiimides, BOP reagent (benzotriazol-1-yloxy-tris(dimethylamino)-phosphonium hexafluorophosphonate) and the like. Suitable carbodiimides include, by way of example, dicyclohexylcarbodiimide (DCC), 1-(3-dimethylaminopropyl)-3-ethylcarbodiimide (EDC) and the like. If desired, polymer supported forms of carbodiimide coupling reagents may also be used including, for example, those described in Tetrahedron Letters, 34(48), 7685 (1993). Additionally, well-known coupling promoters, such as N-hydroxysuccinimide, 1-hydroxybenzotriazole and the like, may be used to facilitate the coupling reaction.

This coupling reaction is typically conducted by contacting the N-sulfonylamino acid, 3, with about 1 to about 2 equivalents of the coupling reagent and at least one equivalent, preferably about 1 to about 1.2 equivalents, of tyrosine derivative, 4, in an inert diluent, such as dichloromethane, chloroform, acetonitrile, tetrahydrofuran, N,N-dimethylformamide and the like. Generally, this reaction is conducted at a temperature ranging from about 0° C. to about 37° C. for about 12 to about 24 hours. Upon completion of the reaction, the compound 5 is recovered by conventional methods including neutralization, evaporation, extraction, precipitation, chromatography, filtration, and the like.

Alternatively, the N-sulfonyl amino acid, 3, can be converted into an acid halide which is then coupled with compound, 4, to provide compound 5. The acid halide can be prepared by contacting compound 3 with an inorganic acid halide, such as thionyl chloride, phosphorous trichloride, phosphorous tribromide or phosphorous pentachloride, or preferably, with oxalyl chloride under conventional conditions. Generally, this reaction is conducted using about 1 to 5 molar equivalents of the inorganic acid halide or oxalyl chloride, either neat or in an inert solvent, such as dichloromethane or carbon tetrachloride, at temperature in the range of about 0° C. to about 80° C. for about 1 to about 48 hours. A catalyst, such as DMF, may also be used in this reaction.

The acid halide of N-sulfonyl amino acid, 3, is then contacted with at least one equivalent, preferably about 1.1 to about 1.5 equivalents, of the tyrosine derivative, 4, in an inert diluent, such as dichloromethane, at a temperature ranging from about −70° C. to about 40° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like. Upon completion of the reaction, compound 5 is recovered by conventional methods including neutralization, evaporation, extraction, precipitation, chromatography, filtration, and the like.

Aternatively, compound 5 can be prepared by first forming a diamino acid derivative and then coupling the diamino acid to the arylsulfonyl halide, 2, as shown in Scheme 3 below: 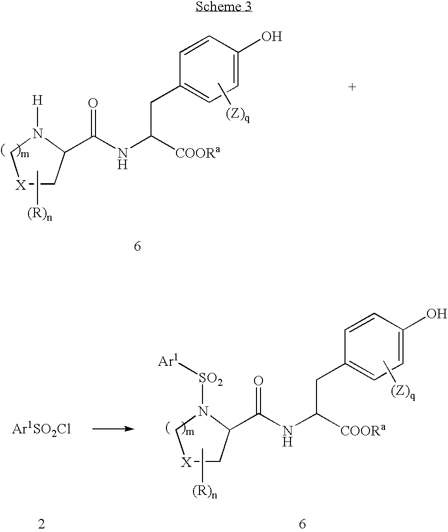
where R, R^{a}, Ar¹, X, Z, m, n and q are as defined above.

The diamino acid, 6, can be readily prepared by coupling amino acid, 1, with amino acid, 4, using conventional amino acid coupling techniques and reagents, such carbodiimides, BOP reagent and the like, as described above. Diamino acid, 6, can then be sulfonated using sulfonyl chloride, 2, and using the synthetic procedures described above to provide compound 7.

The tyrosine derivatives, 4, employed in the above reactions are either known compounds or compounds that can be prepared from known compounds by conventional synthetic procedures. For example, tyrosine derivatives, 4, suitable for use in the above reactions include, but are not limited to, L-tyrosine methyl ester, L-tyrosine t-butyl ester, L-3,5-diiodotyrosine methyl ester, L-3-iodotyrosine methyl ester, β-(4-hydroxy-naphth-1-yl)-L-alanine methyl ester, β-(6-hydroxy-naphth-2-yl)-L-alanine methyl ester, and the like. If desired, of course, other esters or amides of the above-described compounds may also be employed.

The N-arylsulfonyl-heterocyclic amino acid-tyrosine derivative, 7, can be used as a starting point to prepare PEG derivatives at the Ar² group by coupling reactions shown in Schemes 4-14 below which coupling reactions are illustrative only in demonstrating how PEG moieties can be introduced. In some cases, the PEG moiety can be directly introduced onto the phenoxy group and, in other cases, the PEG moiety can be introduced by linkage through a linker moiety.

Specifically, Scheme 4 illustrates the following: 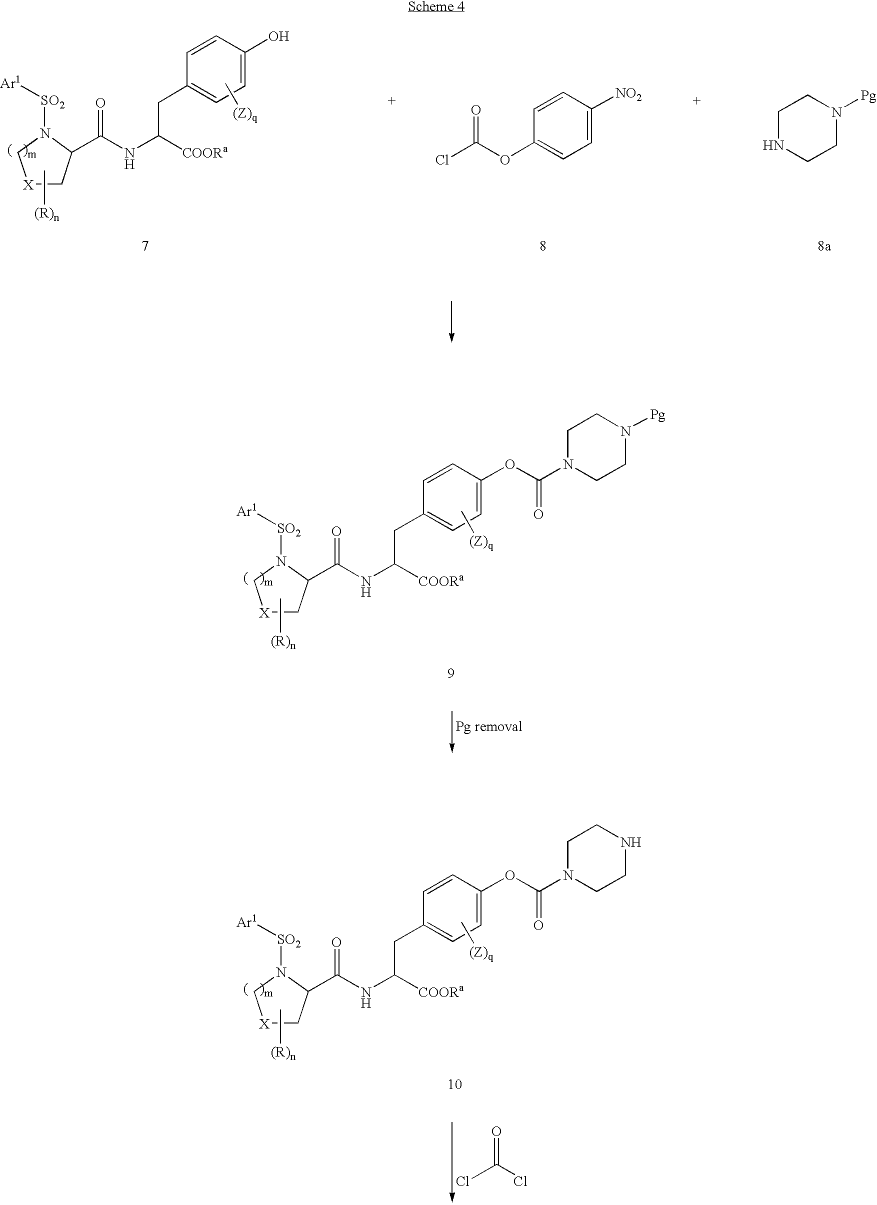 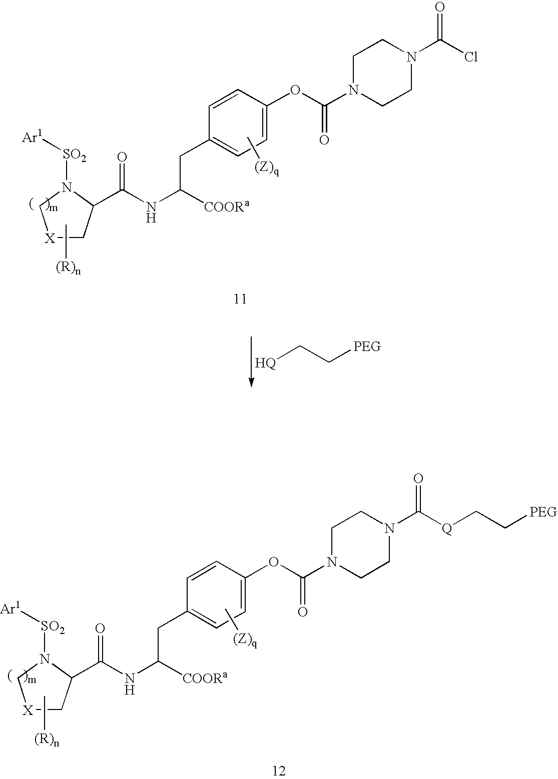
wherein Ar¹, R, R^{a}, m, n, q, X, and Z are as defined above whereas Q is oxygen, sulfur and NH, Pg is an amine protecting group such as CBZ, Boc, etc, which is preferably orthogonally removeable as compared to the R^{a} carboxyl protecting group and PEG is preferably a methyl capped poly(oxyethylene) group having a molecular weight of from 100 to 100,000.

In Scheme 4, the PEG moiety is covalently attached to the N-piperazinylcarbonyltyrosine moiety (R²/R³ are joined together with the nitrogen atom attached thereto to form a piperazine ring) via a linker entity which constitutes the group:

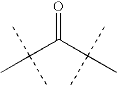

Specifically, in Scheme 4, compound 7, prepared as above, is combined with at least an equivalent and preferably an excess of 4-nitrophenyl chloroformate, 8, in a suitable solvent such as methylene chloride, chloroform and the like and preferably under an inert atmosphere. The reaction is preferably conducted at a temperature of from about −40° to about 0° C. in the presence of a suitable base to scavenge the acid generated. Suitable bases include, by way of example, triethylamine, diisopropylethylamine, and the like. After formation of the intermediate mixed carbonate (not shown), at least an approximately equimolar amount of N-Pg piperazine, 8a, is added to the reaction solution. This reaction is allowed to continue at room temperature for about 1 to 24 hours. Upon completion of the reaction, the compound 9 is recovered by conventional methods including neutralization, evaporation, extraction, precipitation, chromatography, filtration, and the like, or, alternatively, is used in the next reaction without purification and/or isolation.

Conventional removal of the protecting group provides for the free piperazine derivative, 10. Removal is accomplished in accordance with the blocking group employed. For example, a trifluoromethylcarbonyl protecting group is readily removed via an aqueous solution of potassium carbonate. Further, suitable protecting groups for various functional groups as well as suitable conditions for protecting and deprotecting particular functional groups are well known in the art. See, for example, T. W. Greene and G. M. Wuts, Protecting Groups in Organic Chemistry, Second Edition, Wiley, New York, 1991, and references cited therein.

The free piperazine derivative, 10, is then converted to the corresponding carbamyl chloride, 11, by reaction in a biphasic reaction mixture of phosgene in toluene (Fluka), dichloromethane and aqueous bicarbonate solution. Subsequent reaction of the carbamyl chloride, 11, with a mono-capped PEG compound such as commercially available CH₃(OCH₂CH₂)ₚOH provides for PEG derivative 12. The reaction is conducted in a suitable solvent such as methylene chloride, chloroform, etc. typically in the presence of a catalytic amount of DMAP and a base to scavenge the acid generated during reaction. The reaction is continued until substantially complete which typically occurs within 4 to 24 hours.

When R^{a} is alkyl, subsequent hydrolysis of the ester derivative provides for the free carboxyl group or a salt thereof.

A specific example of this reaction scheme up to formation of the piperazine derivative 10 is illustrated in Scheme 5 below:

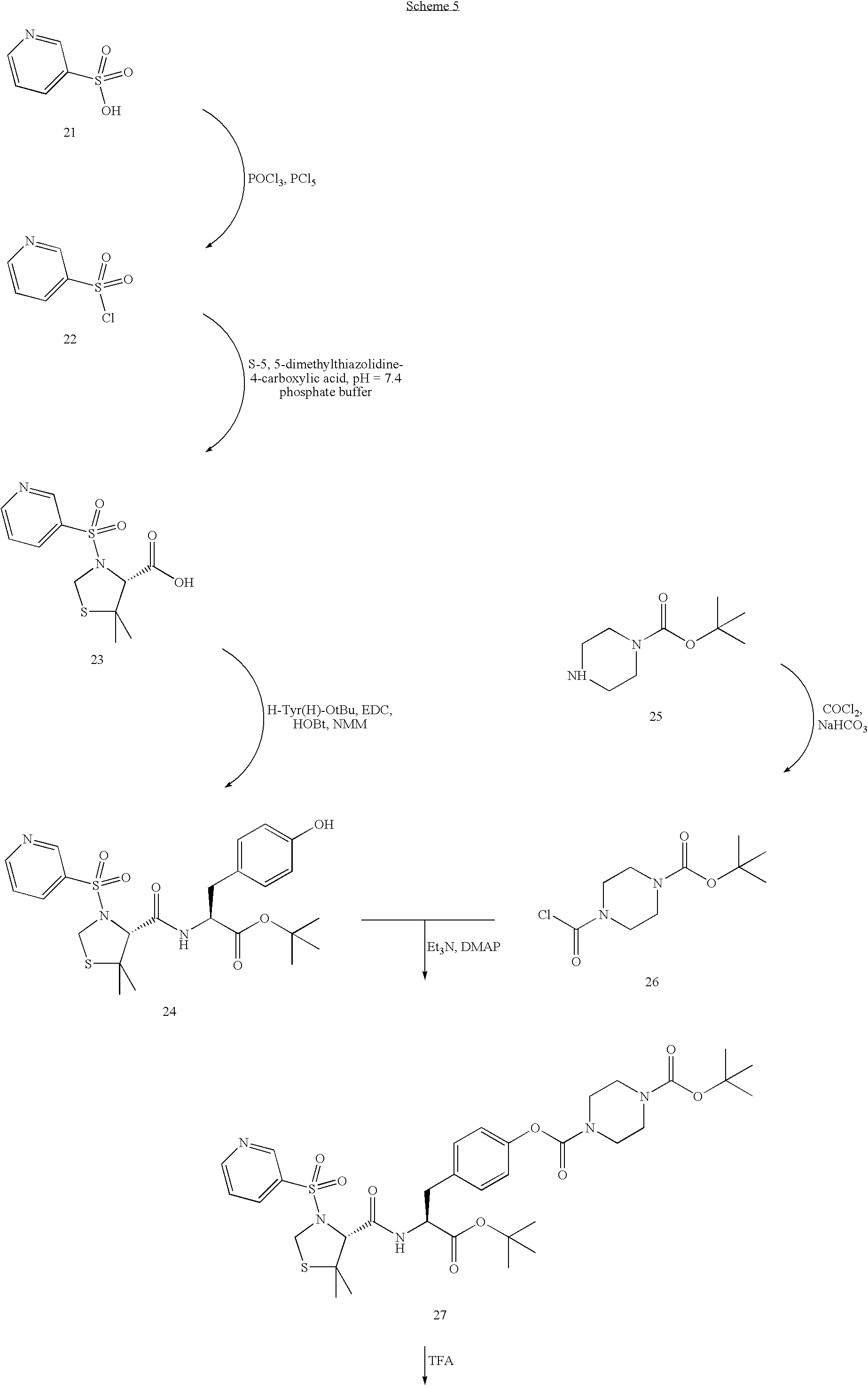

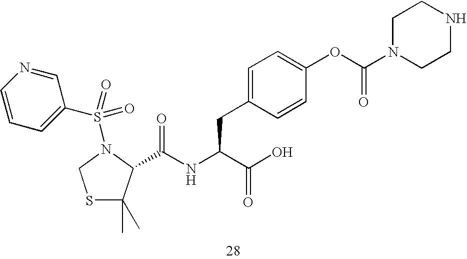

Specifically, commercially available 3-pyridinesulfonic acid, 21, is converted under conventional conditions to the corresponding sulfonyl chloride, 22, by contact with POCl₃/PCl₅ using conditions well known in the art. Coupling of sulfonyl chloride, 22, with commercially available S-5,5-dimethylthiazolidine-4-carboxylic acid, 23, is accomplished under conventional conditions preferably in the presence of a phosphate buffer (pH 7.4) using an excess of sulfonyl chloride. The reaction is preferably conducted at a temperature of from about −10 to 20° C. until the reaction is substantially complete, which typically occurs within 0.5 to 5 hours. The resulting product, 24, can be recovered by conventional methods, such as chromatography, filtration, evaporation, crystallization, and the like or, alternatively, used in the next step without purification and/or isolation.

The N-pyridyl sulfonyl-5,5-dimethylthiazolidine-4-carboxylic acid compound, 23, is next coupled to t-butyl tyrosine using conventional amino acid coupling conditions. Specifically, this coupling reaction is conducted using well known coupling reagents such as 1-(3-dimethylaminopropyl)-3-ethylcarbodiimide (EDC), 1-hydroxy-benzotriazole (HOBt) and N-methylmorpholine to facilitate the coupling reaction.

This coupling reaction is typically conducted by contacting the N-sulfonylamino acid, 23, with about 1 to about 2 equivalents of the coupling reagent and at least one equivalent, preferably about 1 to about 1.2 equivalents, of tyrosine t-butyl ester in an inert diluent, such as dichloromethane, chloroform, acetonitrile, tetrahydrofuran, N,N-dimethylformamide and the like. Generally, this reaction is conducted at a temperature ranging from about 0° C. to about 22° C. for about 12 to about 24 hours. Upon completion of the reaction, the compound 24 is recovered by conventional methods including neutralization, evaporation, extraction, precipitation, chromatography, filtration, and the like or, alternatively, is employed in the next step without purification and/or isolation.

Separately, mono-N-Boc-piperazine, 25, is converted to the corresponding carbamyl chloride, 26, by reaction with phosgene in the manner described above. Upon completion of the reaction, the compound 26 is recovered by conventional methods including neutralization, evaporation, extraction, precipitation, chromatography, filtration, and the like or, alternatively, is employed in the next step without purification and/or isolation.

Coupling of compound 24 with compound 26 to provide for compound 27 proceeds under conventional conditions in an inert diluent such as dichloromethane, with a catalytic amount of DMAP and preferably in the presence of a base to scavenge the acid generate. The reaction is run at a temperature of about −20 to about 22° C. for about 2 to about 24 hours. Upon completion of the reaction, compound 27 is recovered by conventional methods including neutralization, evaporation, extraction, precipitation, chromatography, filtration, and the like or, alternatively, is employed in the next step without purification and/or isolation.

Removal of both the amino Boc protecting group and the t-butyl ester proceeds in the presence of trifluoroacetic acid to provide for compound 28 which can be recovered by conventional methods including neutralization, evaporation, extraction, precipitation, chromatography, filtration, and the like.

Scheme 6 below illustrates the preparation of a piperazine compound orthogonally protected on one of the amine groups relative to the carboxyl protecting group found on the phenylalanine compound such that after coupling, the piperazine protecting group can be removed differentially from that of the carboxyl protecting group. Such orthogonal protection is necessary if subsequent reactions on the resulting compound require a carboxyl protecting group to avoid undesired side reactions.

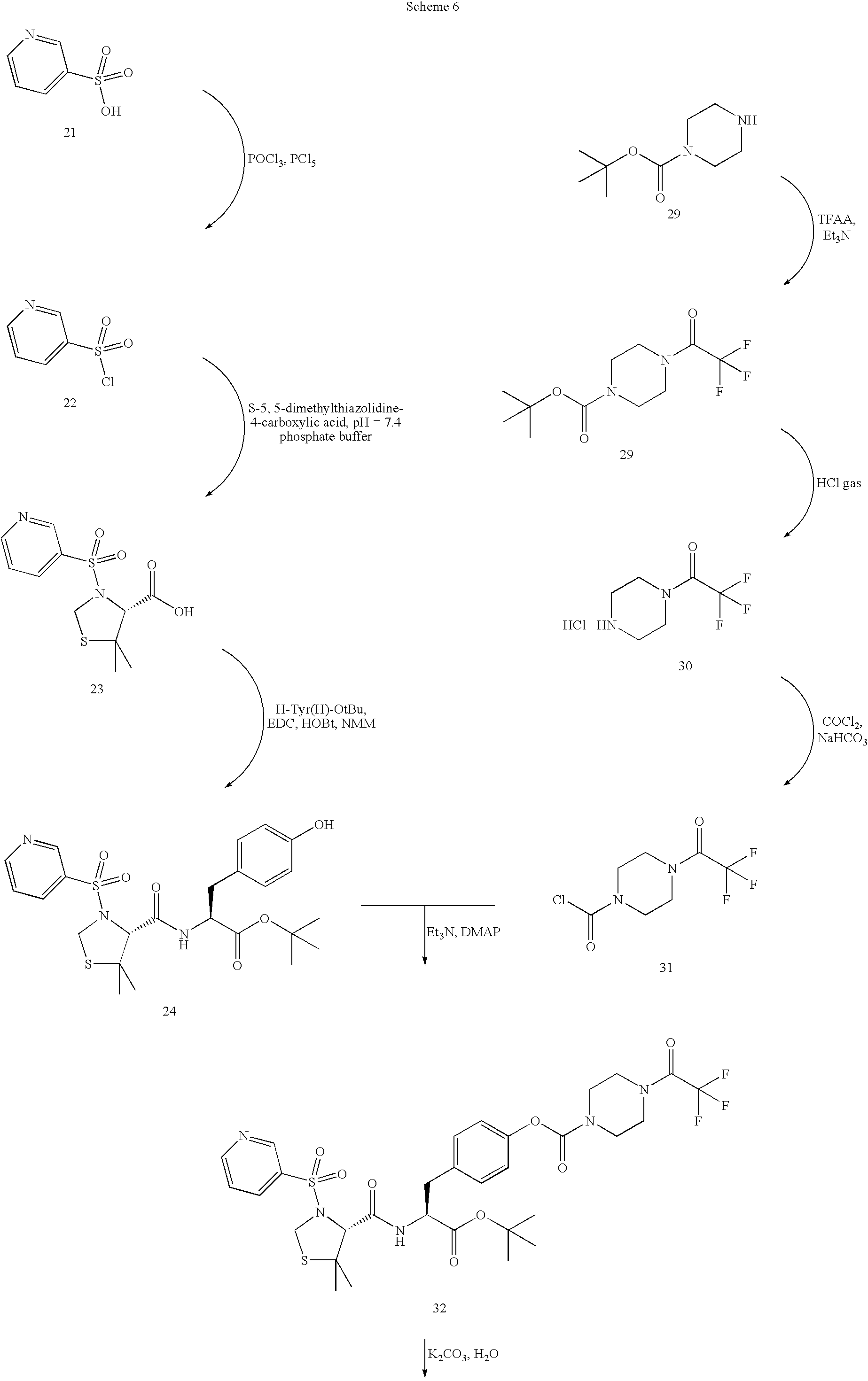

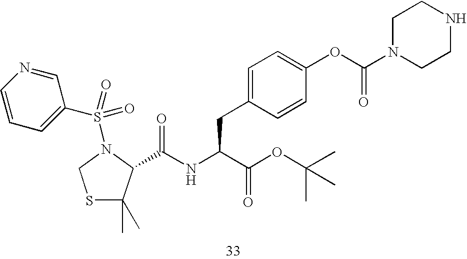

Specifically, in Scheme 6, compound 24 is prepared in the manner described above. N-t-Boc-piperazine, 25, is conventionally converted to N-t-Boc-N′-trifluoromethyl-carbonylpiperazine, 29, by contact with an excess of trifluoroacetic anhydride in the presence of a suitable amine such as triethylamine to scavenge the acid generated during reaction in a suitable solvent such as dichloromethane. Generally, this reaction is conducted at a temperature ranging from about −20° C. to about 22° C. for about 1 to about 24 hours. Upon completion of the reaction, compound 29 can be recovered by conventional methods including neutralization, evaporation, extraction, precipitation, chromatography, filtration, and the like or, alternatively and preferably, is employed in the next step without purification and/or isolation.

In turn, removal of the t-Boc protecting group on the N-t-Boc-N′-trifluoromethylcarbonylpiperazine, 29, proceeds under conventional conditions using gaseous HCl bubbled through an inert solvent such as methylene chloride, EtOAc, EtO₂, and the like under ambient conditions to provide for the hydrochloride salt of N′-trifluoromethylcarbonylpiperazine, 30. Generally, this reaction is conducted at a temperature ranging from about −20° C. to about 22° C. for about 0.5 to about 4 hours. Upon completion of the reaction, compound 30 can be recovered by conventional methods including neutralization, evaporation, extraction, precipitation, chromatography, filtration, and the like or, alternatively and preferably, is employed in the next step without purification and/or isolation.

Conversion of N′-trifluoromethylcarbonylpiperazine, 30, to the N-carbamyl chloride derivative, 31, conventionally proceeds by contact with phosgene in the manner described above. Upon completion of the reaction, compound 31 can be recovered by conventional methods including neutralization, evaporation, extraction, precipitation, chromatography, filtration, and the like or, alternatively and preferably, is employed in the next step without purification and/or isolation.

Compounds 31 and 24 are coupled under conditions similar to those described above to provide for compound 32 which is orthogonally protected at the amino moiety of the piperazine group as well as the carboxyl moiety of the phenylalanine group. Selective removal of the trifluoromethylcarbonyl amino protecting group proceeds under conventional conditions using an aqueous solution of potassium carbonate to provide for compound 33.

Scheme 7 below illustrates a first route for derivatization of compound 28 to provide for PEG substitution. In this scheme, the amino moiety of the piperazine group is employed as a complementary functional group to the activated carboxyl group of the lysine derivative to form a covalent amide bond thereby introducing two PEG moieties into the compound through a linker of the formula 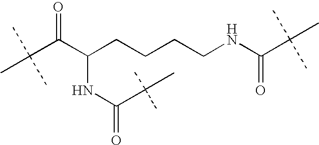
which linker comprises 8 carbon atoms and 5 heteroatoms.

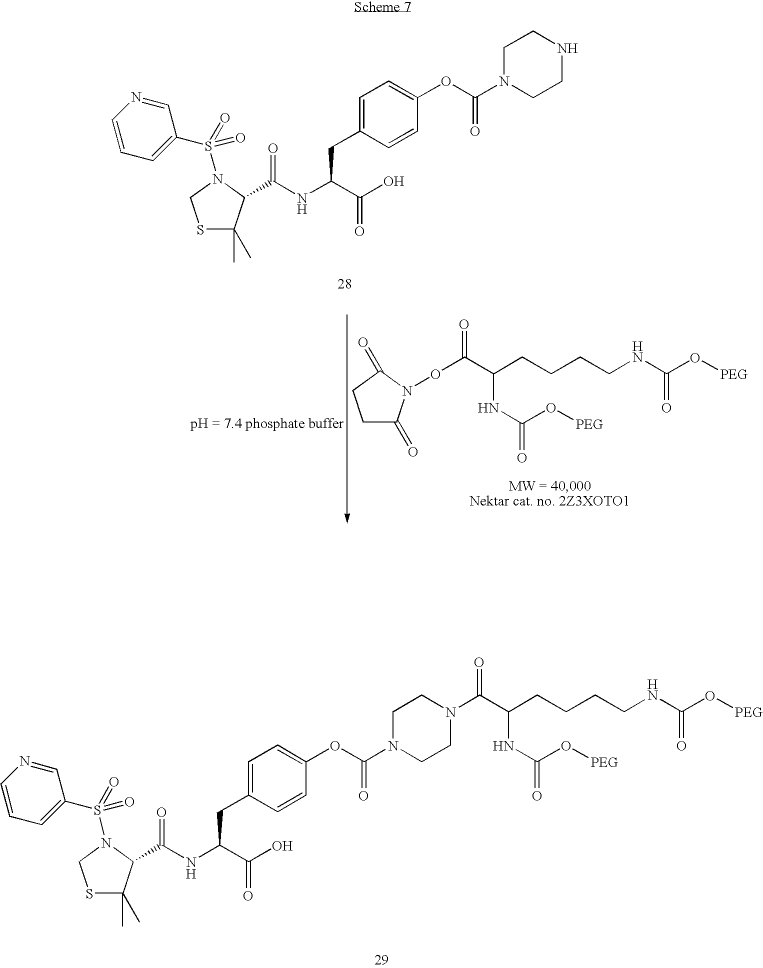

Specifically, in Scheme 7, conjugation of an excess of compound 28 (1.1 to 10 eq) with commercially available N-hydroxysuccinimidyl ester of a di-PEG substituted lysine derivative, in the presence of phosphate buffered aqueous solution provides for compound 29 which is recovered by dialysis. The commercially available N-hydroxy-succinimidyl ester of a di-PEG substituted lysine derivative has a weight average molecular weight of about 40,000 which means that each PEG moiety has a number average molecular weight of about 20,000. The reaction is run at a temperature of about 0 to about 22° C.

Scheme 8 illustrates a second route for derivatization to provide for PEG substitution. In this scheme, the amino moiety of the piperazine group is employed as a complementary functional group to an in situ formed activated carboxyl group of a commercially available carboxyl-PEG compound which under conventional reactive conditions forms a covalent amide bond thereby introducing a single PEG moiety into the compound. In this embodiment, the carboxyl-PEG compound is represented by the formula HOOC(CH₂)ᵥ(OCH₂CH₂)ₚOCH₃ where p and v are as defined above and the resulting linker to the PEG group is represented by —C(O)(CH₂)ᵥ—. Carboxylated PEG compounds can be made by oxidation of the hydroxy terminated PEG compounds using conventional methods and reagents.

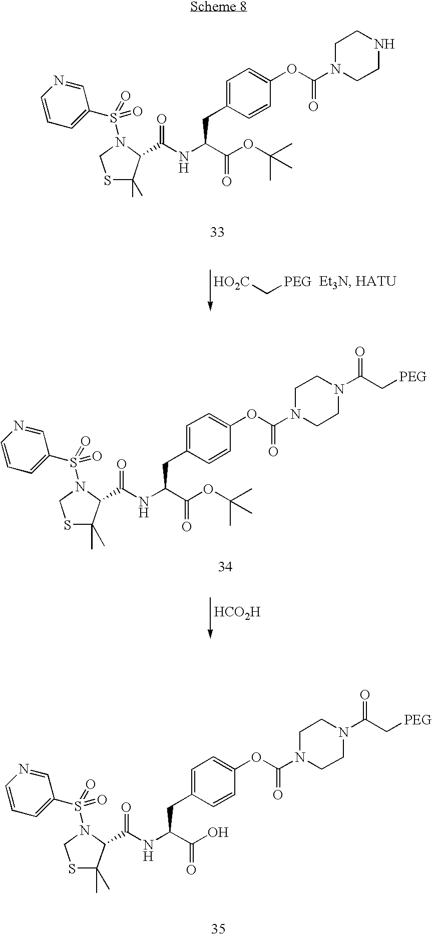

Specifically, in Scheme 8, an excess (1.1 to 10 equiv) of compound 33, prepared as in Scheme 7, is added to at least an equivalent of a commercially available carboxyl-PEG compound which is convertd in situ to an activated ester (not shown) by contact with at least an equivalent and preferably an excess of HATU [O-(7-azabenzotriazol-1-yl)-1,1,3,3,-tetramethyluronium hexafluorophosphate] in the presence of a suitable amine such as triethylamine. Coupling of the carboxyl-PEG compound to compound 33 preferably proceeds at a temperature of from about 0 to about 22° C. for about 2 to about 24 hours. Upon completion of the reaction, the compound 34 is recovered by conventional methods including neutralization, evaporation, extraction, precipitation, chromatography, filtration, and the like or, alternatively, is employed in the next step without purification and/or isolation.

Conventional removal of the t-butyl carboxyl protecting group with an excess of formic acid provides for a mono-PEG compound of formula XXII of this invention.

Scheme 9 illustrates a third route for derivatization to provide for PEG substitution. In this scheme, the amino moiety of the piperazine group is employed as a complementary functional group to an in situ formed chloroformate of a commercially available mono-hydroxy-PEG compound which under conventional reactive conditions forms a covalent carbamate bond thereby introducing a single PEG moiety into the compound. In this embodiment, the mono-hydroxy-PEG compound is represented by the formula HOCH₂CH₂(OCH₂CH₂)ₚOCH₃ where p is as defined above and the resulting linker is represented by —C(O)—.

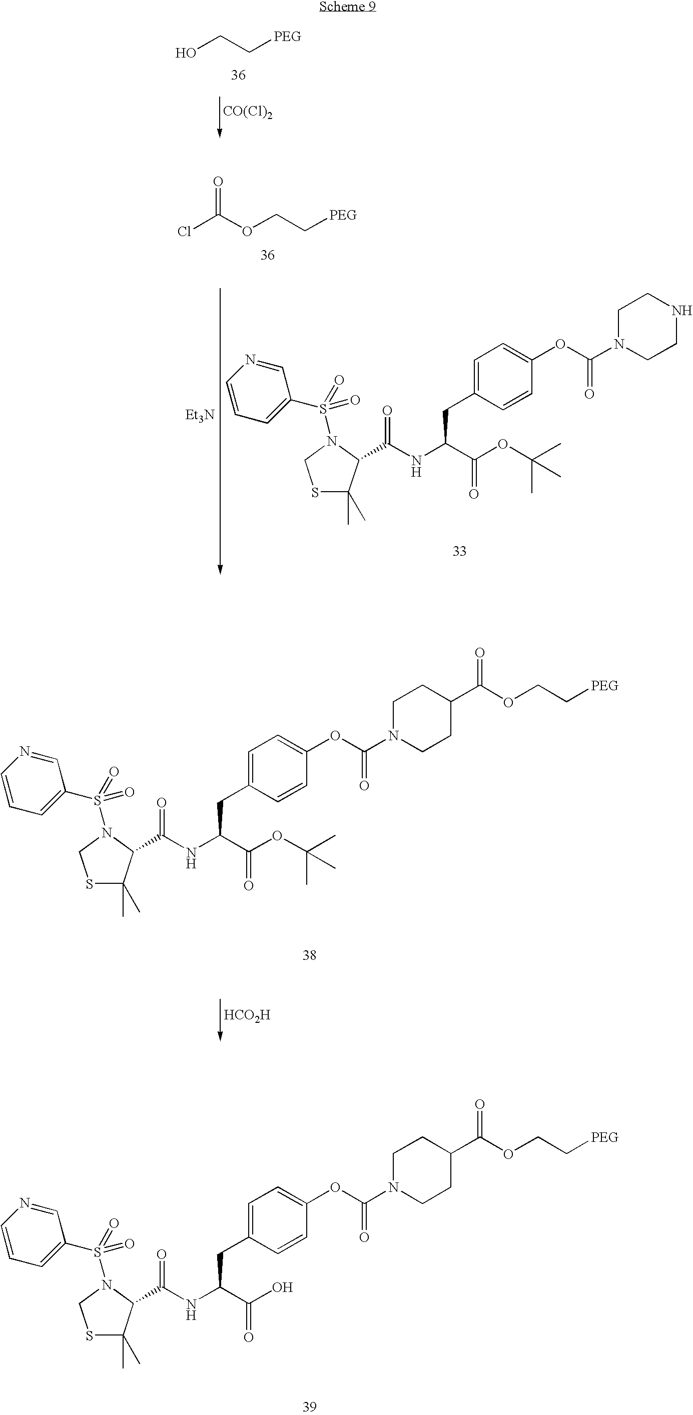

Specifically, in Scheme 9, the hydroxyl group of a commercially available mono-hydroxy PEG, 36, is converted to the corresponding chloroformate, 37, by reaction with phosgene in toluene (Fluka), in dichloromethane. The product is isolated by evaporation and is employed in the next step without further purification.

A slight excess (1.1 to 10 eq) of chloroformate 37 is contacted with compound 33, prepared as above, in the presence of a suitable base such as triethylamine to scavenge the acid generated. Coupling of the chloroformate-PEG compound to compound 33 preferably proceeds at a temperature of from about 0 to about 22° C. for about 2 to about 4 hours. Upon completion of the reaction, the compound 38 is recovered by conventional methods including neutralization, evaporation, extraction, precipitation, chromatography, filtration, and the like or, alternatively, is employed in the next step without purification and/or isolation.

Conventional removal of the t-butyl carboxyl protecting group with an excess of formic acid provides for a mono-PEG compound, 39, of formula XXII of this invention.

Scheme 10 illustrates the synthesis of two intermediates useful for subsequent PEG substitution. In this scheme, the amino moiety of the piperazine group is employed as a complementary functional group which is derivatized for subsequent PEG substitution.

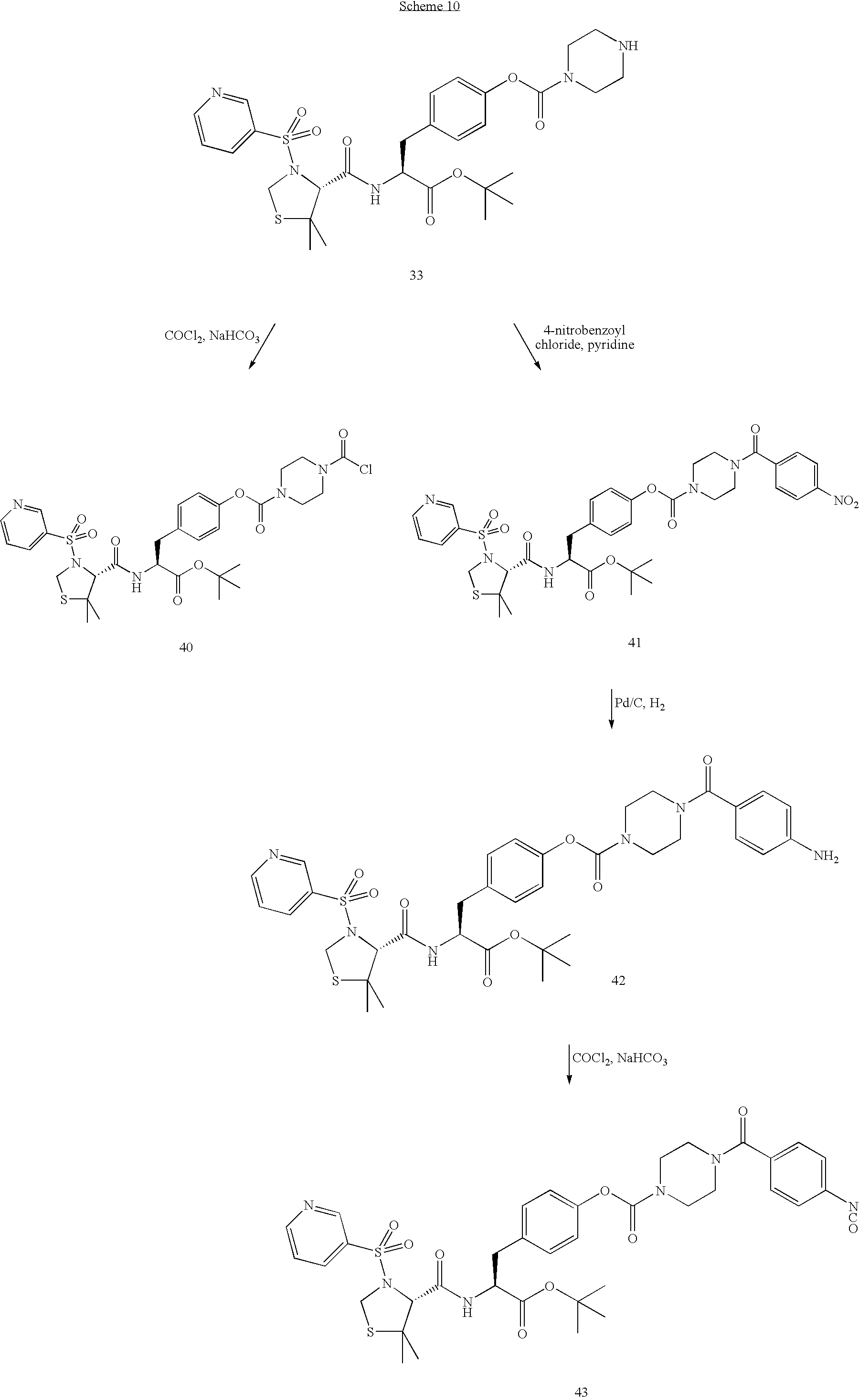

Specifically, in Scheme 10, conversion of amino moiety of the piperazine group to the corresponding N-carbamyl chloride derivative, 40, proceeds by contact with an excess of phosgene in the presence of a suitable base such as sodium bicarbonate to scavenge the acid generated during reaction. Upon completion of the reaction, compound 40 can be recovered by conventional methods including neutralization, evaporation, extraction, precipitation, chromatography, filtration, and the like or, alternatively and preferably, is employed in the next step (illustrated in Scheme 11) without purification and/or isolation.

Alternatively, the amino moiety of the piperazine group of compound 33 can be converted to the corresponding amide, compound 41, by reaction with at least an equivalent and preferably an excess of 4-nitrobenzoyl chloride in the presence of a base such as pyridine (which can also act as a solvent) to scavenge the acid generated during reaction. The reaction preferably proceeds at a temperature of from about 0 to about 22° C. for about 1 to about 24 hours. Upon completion of the reaction, compound 41 is recovered by conventional methods including neutralization, evaporation, extraction, precipitation, chromatography, filtration, and the like or, alternatively, is employed in the next step without purification and/or isolation.

Subsequent reduction of the para-nitro substituent of the phenyl group provides for the amine substituent in compound 42. Reduction is conventionally conducted using palladium/carbon under a hydrogen atmosphere typically at elevated pressures in a suitable diluent such as methanol. The reaction proceeds until substantial completion which typically occurs within about 24 to about 72 hours. During the reaction, additional catalyst is added as required to affect reaction completion. Upon completion of the reaction, the compound 42 is recovered by conventional methods including neutralization, evaporation, extraction, precipitation, chromatography, filtration, and the like or, alternatively, is employed in the next step without purification and/or isolation.

Conversion of the para-amino substituent of the phenyl group of compound 42 to the corresponding isocyanate, 43, occurs by reaction with an excess of phosgene in the presence of a suitable base such as sodium bicarbonate which scavenges the acid generated. The reaction proceeds until substantial completion which typically occurs within about 0.5 to about 5 hours at about 0° C. to about 22° C. Upon completion of the reaction, the compound 43 is recovered by conventional methods including neutralization, evaporation, extraction, precipitation, chromatography, filtration, and the like or, alternatively, is employed in the next step without purification and/or isolation.

Scheme 11 illustrates a fourth route for derivatization to provide for PEG substitution. In this scheme, the carbamyl chloride moiety of the piperazine group of compound 40 is employed as a complementary functional group to form a carbamate or urea bond with a commercially available mono-hydroxy- or mono-amino-PEG compound which under conventional reactive conditions. In this embodiment, the PEG compound is represented by the formula HQCH₂CH₂(OCH₂CH₂)ₚOCH₃ where p and Q are as defined above and the resulting linker is represented by —C(O)—.

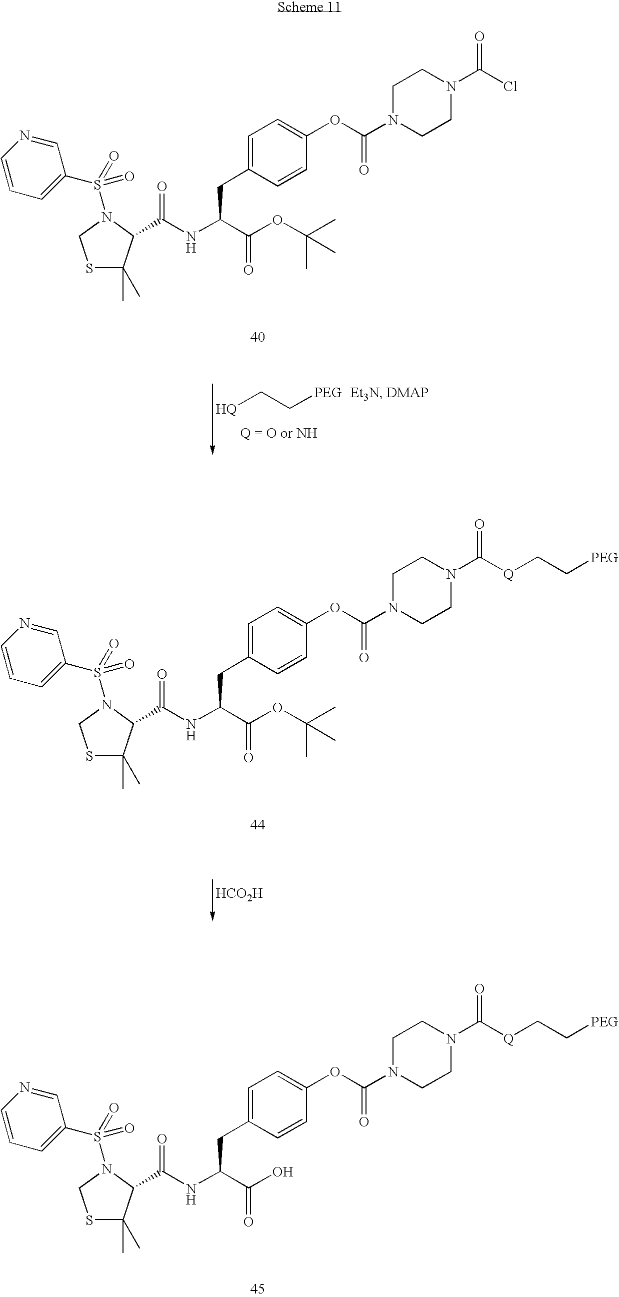

Specifically, in Scheme 11, an excess (1.1 to 10 eq) of carbamyl chloride, 40, is contacted in an inert solvent such as dichloromethane with a suitable mono-hydroxy- or mono-amino-PEG compound preferably in the presence of a suitable base such as triethylamine and/or catalytic amounts of 4-N,N-dimethylaminopyridine (DMAP). The reaction proceeds until substantial completion which typically occurs within about 4 to about 48 hours. Upon completion of the reaction, the compound 44 is recovered by conventional methods including neutralization, evaporation, extraction, precipitation, chromatography, filtration, and the like or, alternatively, is employed in the next step without purification and/or isolation.

When Q is a hydroxyl group, the resulting product contains a carbamate functionality covalently linking the PEG group to the VLA-4 antagonist through a linker represented by —C(O)—. When Q is an amino group, the resulting product contains a urea functionality covalently linking the PEG group to the VLA-4 antagonist through a linker represented by —C(O)—.

Conventional removal of the t-butyl carboxyl protecting group with an excess of formic acid provides for a mono-PEG compound, 45, of formula XXIIa of this invention.

Scheme 12 illustrates a fifth route for derivatization to provide for PEG substitution. In this scheme, the isocyanate moiety of the phenyl group of compound 43 is employed as a complementary functional group to form a carbamate or urea bond with a commercially available mono-hydroxy- or mono-amino-PEG compound which under conventional reactive conditions. In this embodiment, the PEG compound is represented by the formula HQCH₂CH₂(OCH₂CH₂)ₚOCH₃ where p and Q are as defined above and the resulting linker is represented by: 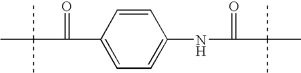
where the linker comprises 8 carbon atoms and 3 heteroatoms.

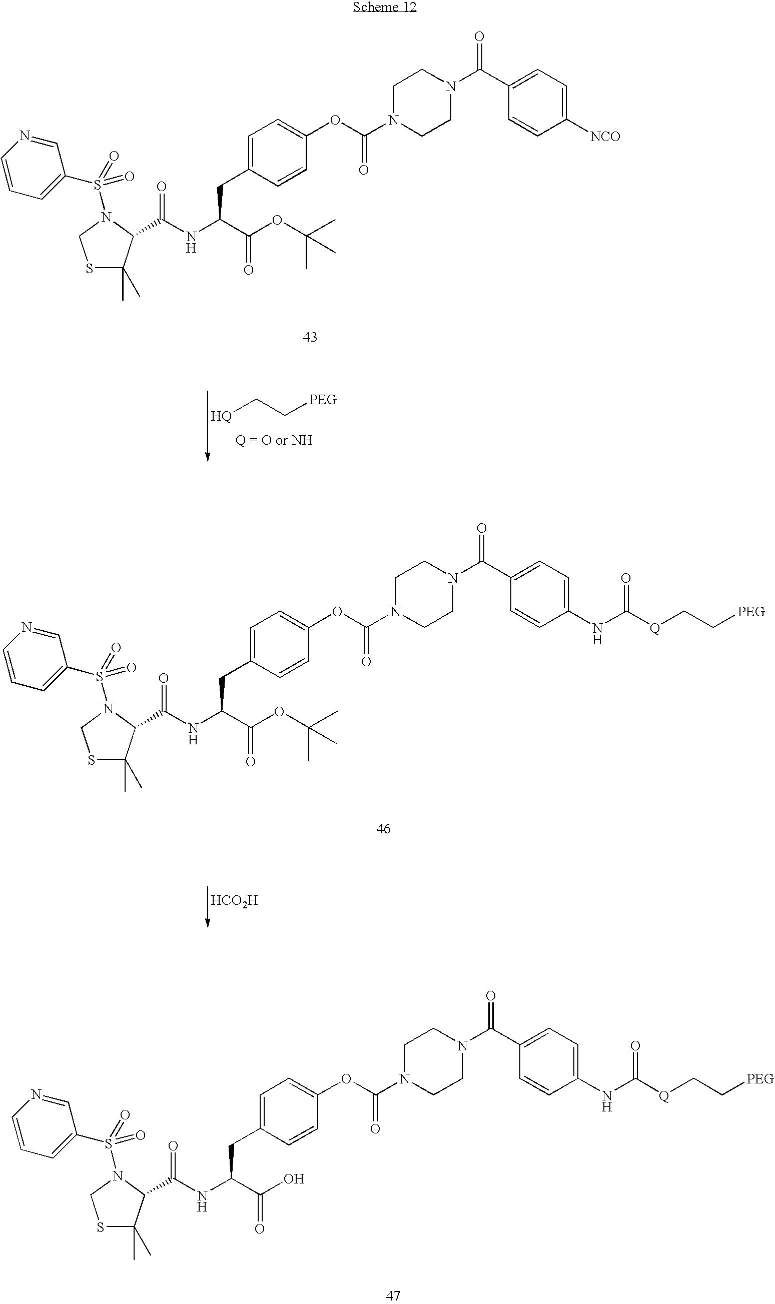

Specifically, in Scheme 12, an excess (1.1 to 10 eq) isocyanate, 43, is contacted with a suitable mono-hydroxy- or mono-amino-PEG compound in a suitable inert diluent such as dichloromethane or toluene. The reaction is preferably maintained at a temperature of from about 0° to about 105° C. until substantial completion which typically occurs within about 1 to about 24 hours. Upon completion of the reaction, compound 46 is recovered by conventional methods including neutralization, evaporation, extraction, precipitation, chromatography, filtration, and the like or, alternatively, is employed in the next step without purification and/or isolation.

When Q is a hydroxyl group, the resulting product contains a carbamate functionality covalently linking the PEG group to the VLA-4 antagonist through a —C(O)— linking group. When Q is an amino group, the resulting product contains a urea functionality covalently linking the PEG group to the VLA-4 antagonist through a —C(O)— linking group.

Conventional removal of the t-butyl carboxyl protecting group with an excess of formic acid provides for a mono-PEG compound, 47, of formula XXII of this invention.

In the schemes above, amine moieties located on other portions of the molecule can be employed in the manner described above to covalently link a PEG group to the molecule. For example, amines located on Ar¹, on the heterocyclic amino acid or on Ar² can be similarly derivatized to provide for PEG substitution. The amine moieties can be included in these substituents during synthesis and appropriately protected as necessary. Alternatively, amine precursors can be employed. For example, as shown in Scheme 10, reduction of a nitro group provides for the corresponding amine. Similarly, reduction of a cyano group provides for a H₂NCH₂— group. Nitro and cyano substituted Ar¹ groups are provided in U.S. Pat. No. 6,489,300 as is an amino substituted Ar¹ group.

Further, the amino substitution can be incorporated into the heterocyclic amino acid functionality and then derivatized to include a PEG moiety found in formula XXII as R. For example, the heterocyclic amino acid functionality can be 2-carboxylpiperazine depicted in U.S. Pat. No. 6,489,300. Alternatively, commercially available 3- or 4-hydroxyproline can be oxidized to the corresponding ketone and then reductively aminated with ammonia in the presence of sodium cyanoborohydride to form the corresponding amine moiety. Still further, 4-cyanoproline can be reduced to provide for a substituted alkyl group of the formula —CH₂NH₂ which can be derivatized through the amine.

Still further, the amine moiety can be incorporated into the Ar² functionality. Preferably, the amine moiety is present as an amine precursor such as a nitro or cyano group bound to Ar².

In the schemes above, the reactions of the amine with a complementary functional group can be reversed such that the carboxyl or hydroxyl group is on the VLA-4 antagonist of formula XXIIa (without any PEG substituents) and the amine group could be part of the PEG moiety. In such cases, the amine group, preferably terminating the PEG moiety, can be converted to an isocyanate, using phosgene and Et₃N, and reacted with the hydroxyl group to form a carbamate as illustrated in Scheme 13 below:

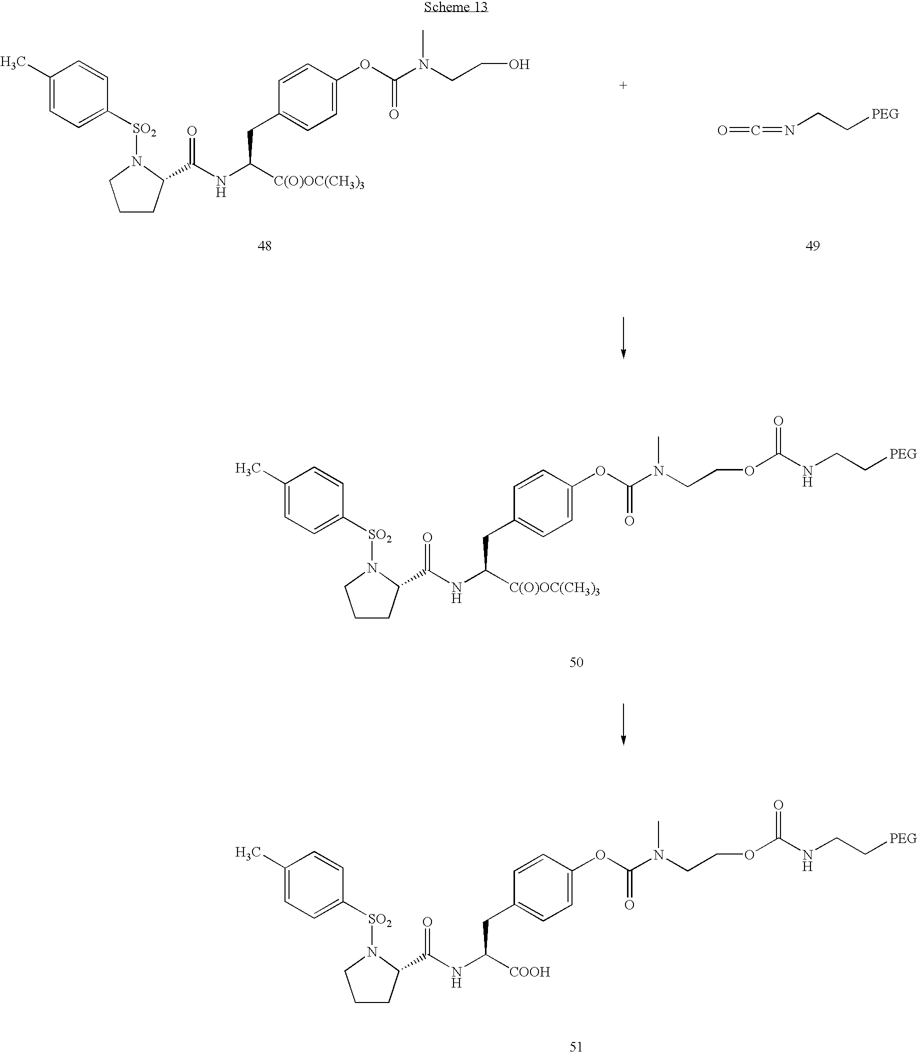

Specifically, compound 48 described in U.S. Pat. No. 6,489,300 is contacted with at least an equivalent and preferably an excess of 49 in the manner described above to provide for the corresponding carbamate, 50. Deprotection, as described above, then provides for compound 51.

Alternatively, in Scheme 13, the hydroxyl functionality can be reacted with phosgene to provide for the chlorocarbonyloxy derivative which reacts with an amine group of a monoamine compound to provide for the carbamate.

Carboxyl functionality, for example on the Ar¹ moiety, can be converted to the corresponding amide by reaction with a mono-amino-PEG compound in the manner described above in Scheme 8.

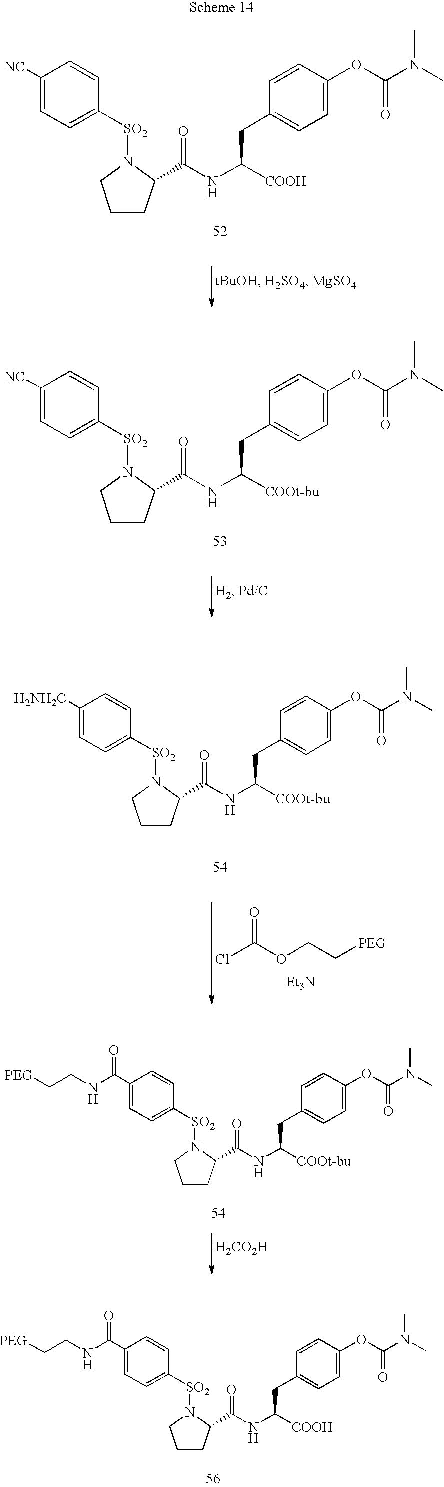

Specifically, in Scheme 14, known compound 52, described in U.S. Pat. No. 6,489,300, is t-butyl protected under convention conditions to provide the cyano compound 53, which is hydrogenated under conventional conditions to provide the aminomethyl compound 54. The aminomethyl group is reacted with Et₃N and a PEG chloroformate, as illustrated previously in Scheme 9, to provide the carbamate-linked conjugate t-butyl ester 55. Treatment of the t-butyl ester with HCO₂H provides the conjugate carboxylic acid 56.

Suitable PEG compounds are commercially available or can be prepared by art recognized procedures. For example, mono-capped linear PEGs with one terminal amine are available in varying molecular weights (e.g., 2 kilodaltons (kDa), 5 kDa, 10 kDa and 20 kDa from Nektar, San Carlos, Calif.). Preferred mono-capped PEGs having one terminal amine group can be represented by the formula H₂NCH₂CH₂(OCH₂CH₂)ₚOCH₃.

Mono-capped linear PEGs with one terminal alcohol are available in varying molecular weights (e.g., 2 kilodaltons (kDa), 5 kDa, 10 kDa and 20 kDa from Nektar, San Carlos, CA). Preferred mono-capped linear PEGs having one terminal alcohol can be represented by the formula HOCH₂CH₂(OCH₂CH₂)ₚOCH₃.

Diamino-capped linear PEGs having an amino group at both termini are commercially available and are sometimes referred to as “Jeffamines” (tradename of Huntsman). Preferred diamino-capped linear PEGs having an amino group at both termini can be represented by the formula: H₂NCH₂CH₂(OCH₂CH₂)ₚNH₂.

Scheme 15 below illustrates an alternative synthesis of 3-aminopyrrolidinyl derivatives useful as starting materials in this invention for subsequent PEG substitution at the amino group.

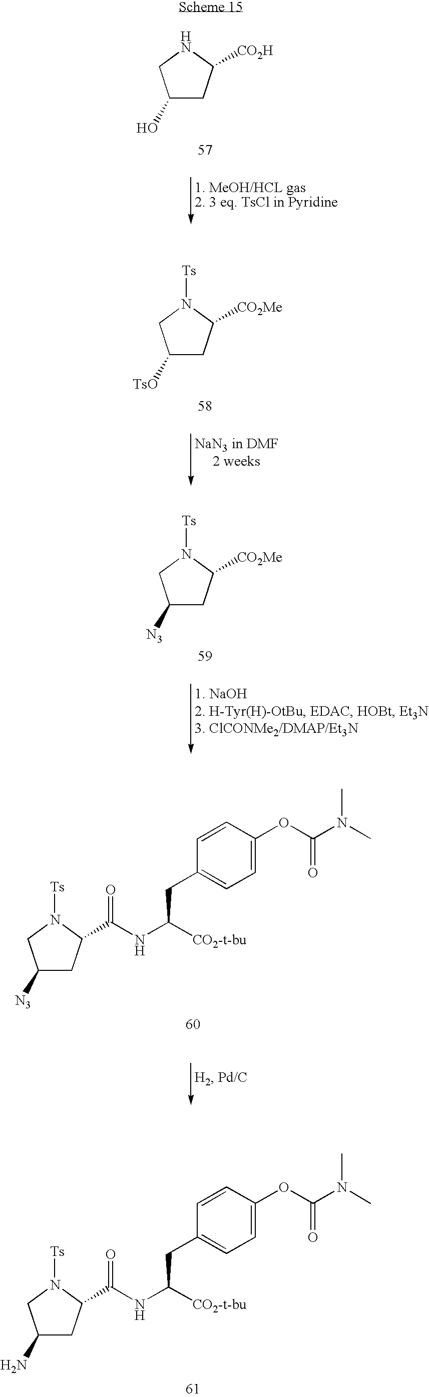

Using conventional methods, commercially available cis-4-hydroxy L-proline, 57, is treated with methanolic hydrogen chloride for several hours at reflux, followed by evaporation, and the so generated methyl ester hydrochloride is treated with excess tosyl chloride in pyridine for two days at room temperature, giving the product, 58. Compound 58 is isolated by neutralizing the pyridine using weak aqueous acid and extracting the product with an organic solvent such as EtOAc. The product 58 may be purified by crystallization, flash chromatography, or more preferably be used in subsequent steps without purification.

Reaction of 58 with a saturated solution of excess sodium azide in DMF at room temperature for 15 days affords compound 59. Compound 59 is isolated by dilution of the reaction mixture with water, followed by extraction with an organic solvent such as EtOAc. The product 59 may be purified by crystallization, flash chromatography, or more preferably be used in subsequent steps without purification.

Compound 59 is treated with sodium hydroxide, in a mixture of water and methanol, thus hydrolyzing the methyl ester and generating a carboxylic acid, which is isolated by acidification and extraction with an organic solvent such as EtOAc. The carboxylic acid is treated with L-tyrosine t-butyl ester [H-Tyr(H)-OtBu], EDAC, HOBt, and Et3N in DMF, generating a dipeptide, which is isolated by dilution with water and extraction with an organic solvent such as EtOAc. The dipeptide is treated with ClCONMe2, Et3N, and DMAP in DCM at reflux for 24 hours, generating the carbamate, 60, which is isolated by dilution with EtOAc, sequential washing with weak aqueous acid and base, and then evaporation. Compound 60 is rigorously purified by flash chromatography.

Finally, compound 61 is prepared by shaking of a solution of 60 in methanol, with a Pd/C catalyst under an atmosphere of hydrogen. The product, 61, is isolated by removal of the catalyst by filtration and evaporation.

Still further, the synthesis of varying mono-capped mono-hydroxy PEGs are described in detail by Campbell, U.S. Pat. No. 4,604,103 which is incorporated herein by reference in its entirety. If a mono-capped mono-amino PEG is preferred, the mono-capped mono-hydroxy PEGs can readily be converted to the corresponding chloride by conventional methods and subsequently converted to an amine by contact with an excess of ammonia.

The PEGs of this invention comprise, for example, the following:

| | |
|---|---|
| HO(alkylene-O)ₚH | dihydroxy-PEG |
| HO(alkylene-O)ₚR^{b} | mono-capped mono-hydroxy PEG |
| H₂N(alkylene-O)ₚR^{b} | mono-capped mono-amino PEG |
| H₂N(alkylene-O)ₚCH₂CH₂NH₂ | Jeffamines |

where p and alkylene are as defined herein and R^{b} is preferably selected from the group consisting of alkyl, substituted alkyl, aryl and substituted aryl.

The PEG derivatives described herein can be used in the pharmaceuticals formulations described above. Preferably, the formulations are administered orally or parenterally to a subject in need thereof.

6. Immunoglobulins

In one specific embodiment, the agents of the invention are immunoglobulins the when administered to a patient may be used in the diagnosis and treatment of inflammatory bowel disease, asthma, multiple sclerosis (MS), rheumatoid arthritis (RA), graft versus host disease (GVHD), host versus graft disease, and various spondyloarthropathies, such that a patient previously taking steroids may be tapered off the steroids and/or discontinued from them. These immunoglobulins may be selected from immunoglobulins that selectively bind to an α₄ integrin or a dimer comprising α₄ integrin, such as α₄β₁, or bind VCAM-1. Preferably, the immunoglobulins bind α₄β₁ or α₄β₇ and inhibits α₄β₁ or α₄β₇ activity. The immunoglobulins are preferably antibodies or fragments thereof.

By “antibodies” is meant to include complete immunoglobulins such as IgG1 (or any IgG subclass) or IgM, or inhibitors derived from antibodies, such as natalizumab (Tysabri®).

By “antibody homolog” is meant to include intact antibodies consisting of immunoglobulin light and heavy chains linked via disulfide bonds. The term “antibody homolog” is also intended to encompass a protein comprising one or more polypeptides selected from immunoglobulin light chains, immunoglobulin heavy chains and antigen-binding fragments thereof which are capable of binding to one or more antigens (i.e., integrin or integrin ligand). The component polypeptides of an antibody homolog composed of more than one polypeptide may optionally be disulfide-bound or otherwise covalently crosslinked. Accordingly, therefore, “antibody homologs” include intact immunoglobulins of types IgA, IgG, IgE, IgD, IgM (as well as subtypes thereof, e.g., IgG1), wherein the light chains of the immunoglobulin may be of types kappa or lambda. “Antibody homologs” also includes portions of intact antibodies that retain antigen-binding specificity, for example Fab fragments, Fab′ fragments, F(ab′)₂ fragments, Fv fragments, scFv fragments, heavy and light chain monomers, dimers, derivatives, or mixtures thereof.

When the agent of the invention is an antibody, a monoclonal antibody is the preferred antibody. In contrast to polyclonal antibody preparations, which typically include different antibodies directed against different epitopes, each monoclonal antibody is directed against a single epitope on the antigen. A second advantage of monoclonal antibodies is that they are synthesized by means that are uncontaminated by other immunoglobulins, e.g., by phage display or isolation from a hybridoma. Although the present invention intends to encompass both polyclonal and monoclonal antibodies as agents of the invention, monoclonal antibodies are preferred as they are highly specific, and the invention is thus discussed primarily in terms of monoclonal antibodies.

“Native antibodies and immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one and (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., 1985, J. Mol. Biol., 186: 651-63; Novotny et al., 1985, Proc. Natl. Acad. Sci. USA, 82: 4592-6).

In addition, other antibodies can be identified using techniques available in the art. For example, monoclonal antibodies of the present invention can be produced using phage display technology. Antibody fragments, which selectively bind to an α₄ integrin or a dimer comprising an α₄ integrin, are then isolated. Exemplary preferred methods for producing such antibodies via phage display are disclosed in U.S. Pat. Nos. 6,225,447; 6,180,336; 6,172,197; 6,140,471; 5,969,108; 5,885,793; 5,872,215; 5,871,907; 5,858,657; 5,837,242; 5,733,743 and 5,565,332.

A “variant” antibody refers herein to an immunoglobulin molecule that differs in amino acid sequence from a “parent” antibody amino acid sequence by virtue of addition, deletion and/or substitution of one or more amino acid residue(s) in the parent antibody sequence. The parent antibody or immunoglobulin can be a polyclonal antibody, monoclonal antibody, humanized antibody, primatized® antibody or any antibody fragment. In the preferred embodiment, the variant comprises one or more amino acid substitution(s) in one or more hypervariable region(s) of the parent antibody. For example, the variant may comprise at least one, e.g., from about one to about ten, and preferably from about two to about five, substitutions in one or more hypervariable regions of the parent antibody. Ordinarily, the variant will have an amino acid sequence having at least 75% amino acid sequence identity with the parent antibody heavy or light chain variable domain sequences, more preferably at least 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. No N-terminal, C-terminal, or internal extensions, deletions, or insertions into the antibody sequence shall be construed as affecting sequence identity or homology. The variant retains the ability to bind the receptor and preferably has properties that are superior to those of the parent antibody. For example, the variant may have a stronger binding affinity, enhanced ability to activate the receptor, etc. To analyze such properties, one should compare a Fab form of the variant to a Fab form of the parent antibody or a full-length form of the variant to a full-length form of the parent antibody. The variant antibody of particular interest herein is one which displays at least about 10 fold, preferably at least about 20 fold, and most preferably at least about 50 fold, enhancement in biological activity when compared to the parent antibody. The “parent” antibody herein is one that is encoded by an amino acid sequence used for the preparation of the variant. Preferably, the parent antibody has a human framework region and has human antibody constant region(s). For example, the parent antibody may be a humanized or human antibody. An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibodies will be prepared by at least one purification step.

6.1 Monoclonal Antibodies

Monoclonal antibodies can also be produced using the conventional hybridoma methods or genetically engineered. These methods have been widely applied to produce hybrid cell lines that secrete high levels of monoclonal antibodies against many specific antigens, and can also be used to produce monoclonal antibodies of the present invention. For example, mice (e.g., Balb/c mice) can be immunized with an antigenic α₄ epitope by intraperitoneal injection. After sufficient time has passed to allow for an immune response, the mice are sacrificed and the spleen cells obtained and fused with myeloma cells, using techniques well known in the art. The resulting fused cells, hybridomas, are then grown in a selective medium, and the surviving cells grown in such medium using limiting dilution conditions. After cloning and recloning, hybridomas can be isolated that secrete antibodies (for example, of the IgG or IgM class or IgG1 subclass) that selectively bind to the target, α₄ or a dimer comprising an α₄ integrin. To produce agents specific for human use, the isolated monoclonal can then be used to produce chimeric and humanized antibodies. Antibodies can also be prepared that are anti-peptide antibodies. Such anti-peptide antibodies would be prepared against peptides of α₄ integrin.

The term “chimeric”, when referring to an agent of the invention, means that the agent is comprised of a linkage (chemical cross-linkage or covalent or other type) of two or more proteins having disparate structures and/or having disparate sources of origin. Thus, a chimeric α₄ integrin antagonist may include one moiety that is an α₄ integrin antagonist or fragment and another moiety that is not an α₄β₁ integrin antagonist.

A species of “chimeric” protein is a “fusion” or “fusion protein” refers to a co-linear, covalent linkage of two or more proteins or fragments thereof via their individual peptide backbones, most preferably through genetic expression of a polynucleotide molecule encoding those proteins. Thus, preferred fusion proteins are chimeric proteins that include an antibody or fragment thereof covalently linked to a second moiety that is not original to the antibody (i.e., which derives from another immuoglobulin or polypeptide). Preferred fusion proteins of the invention may include portions of intact antibodies that retain antigen-binding specificity, for example, Fab fragments, Fab′ fragments, F(ab′)₂ fragments, Fv fragments, scFv fragments, heavy chain monomers or dimers, light chain monomers or dimers, dimers consisting of one heavy and one light chain, and the like.

The most preferred fusion proteins are chimeric and comprise a moiety fused or otherwise linked to all or part of the hinge and constant regions of an immunoglobulin light chain, heavy chain, or both. Thus, this invention features a molecule which includes: (1) first moiety, (2) a second peptide, e.g., one which increases solubility or in vivo life time of the moiety, e.g., a member of the immunoglobulin super family or fragment or portion thereof, e.g., a portion or a fragment of IgG, e.g., the human IgG1 heavy chain constant region, e.g., CH₂, CH₃, and hinge regions. Specifically, a “steroid sparing/Ig fusion” is a protein comprising a biologically active steroid sparing moiety of the invention. A species of agents is an “integrin/Fc fusion” which is a protein comprising a steroid sparing immunoglobulin of the invention linked to at least a part of the constant domain of an immunoglobulin. A preferred Fc fusion comprises an steroid sparing immunoglobulin of the invention linked to a fragment of an antibody containing the C terminal domain of the heavy immunoglobulin chains.

The term “fusion protein” also means a steroid sparing moiety that is chemically linked via a mono- or hetero-functional molecule to a second moiety that is not a steroid sparing moiety (resulting in a “chimeric” molecule) and is made de novo from purified protein as described below. Thus, one example of a chemically linked, as opposed to recombinantly linked, chimeric molecule that is a fusion protein may comprise: (1) an α₄ integrin subunit targeting moiety, e.g., a VCAM-1 moiety capable of binding to VLA-4) on the surface of VLA-4 bearing cells; (2) a second molecule which increases solubility or in vivo life time of the targeting moiety, e.g., a polyalkylene glycol polymer such as polyethylene glycol (PEG). The α₄ targeting moiety can be any naturally occurring α₄ ligand or fragment thereof, e.g., a VCAM-1 peptide or a similar conservatively substituted amino acid sequence.

Chimeric, primatized® and humanized antibodies can be produced from non-human antibodies, and can have the same or similar binding affinity as the antibody from which they are produced. Techniques developed for the production of chimeric antibodies (Morrison et al., 1984 Proc. Natl. Acad. Sci. 81: 6851; Neuberger et al., 1984 Nature 312: 604; Takeda et al., 1985 Nature 314: 452) by splicing the genes from a mouse antibody molecule of appropriate antigen specificity together with genes from, for example, a human antibody molecule of appropriate biological activity can be used; such antibodies are within the scope of this invention. For example, a nucleic acid encoding a variable (V) region of a mouse monoclonal antibody can be joined to a nucleic acid encoding a human constant (C) region, e.g., IgG1 or IgG4. The resulting antibody is thus a species hybrid, generally with the antigen binding domain from the non-human antibody and the C or effector domain from a human antibody.

Humanized antibodies are antibodies with variable regions that are primarily from a human antibody (the acceptor antibody), but which have complementarity determining regions substantially from a non-human antibody (the donor antibody). See, e.g., Queen et al., 1989 Proc. Natl Acad. Sci. USA 86: 10029-33; WO 90/07861; and U.S. Pat. Nos. 6,054,297; 5,693,761; 5,585,089; 5,530,101 and 5,224,539. The constant region or regions of these antibodies are generally also from a human antibody. The human variable domains are typically chosen from human antibodies having sequences displaying a high homology with the desired non-human variable region binding domains. The heavy and light chain variable residues can be derived from the same antibody, or a different human antibody. In addition, the sequences can be chosen as a consensus of several human antibodies, such as described in WO 92/22653.

Specific amino acids within the human variable region are selected for substitution based on the predicted conformation and antigen binding properties. This can be determined using techniques such as computer modeling, prediction of the behavior and binding properties of amino acids at certain locations within the variable region, and observation of effects of substitution. For example, when an amino acid differs between a non-human variable region and a human variable region, the human variable region can be altered to reflect the amino acid composition of the non-human variable region. Several examples of humanizing anti-α₄ antibodies are described herein.

By “humanized antibody homolog” is meant an antibody homolog, produced by recombinant DNA technology, in which some or all of the amino acids of a human immunoglobulin light or heavy chain that are not required for antigen binding have been substituted for the corresponding amino acids from a nonhuman mammalian immunoglobulin light or heavy chain. A “human antibody homolog” is an antibody homolog in which all the amino acids of an immunoglobulin light or heavy chain (regardless of whether or not they are required for antigen binding) are derived from a human source.

In a specific embodiment, the antibodies used in the chronic dosage regime of the present invention are humanized antibodies as disclosed in U.S. Pat. No. 5,840,299, which is incorporated herein by reference.

In another embodiment, transgenic mice containing human antibody genes can be immunized with an antigenic α₄ structure and hybridoma technology can be used to generate human antibodies that selectively bind to α₄.

Chimeric, human and/or humanized antibodies can be produced by recombinant expression, e.g., expression in human hybridomas (Cole et al., MONOCLONAL ANTIBODIES AND CANCER THERAPY, Alan R. Liss, p. 77 (1985)), in myeloma cells or in Chinese Hamster Ovary (CHO) cells. Alternatively, antibody-coding sequences can be incorporated into vectors suitable for introducing into the genome of animal thereby producing a transgenic animal. One example would be to produce such antibodies in the milk of a transgenic animal such as a bovine. See e.g., U.S. Pat. Nos. 5,849,992 and 5,304,489. Suitable transgenes include trangenes having a promoter and/or enhancer from a mammary gland specific gene, for example casein or β-lactoglobulin.

6.2 Humanized and Primatized® Antibodies

In one embodiment of the invention, humanized (and primatized®) immunoglobulins (or antibodies) specific for the α₄ subunit of VLA-4 are provided, which when administered in an effective amount may be used in the treatment and diagnosis of inflammatory bowel disease such as Crohns's disease, asthma, multiple sclerosis (MS), rheumatoid arthritis (RA), graft versus host disease (GVHD), host versus graft disease, and various spondyloarthropathies such that steroids are not necessary. Humanized and primatized® antibodies are antibodies of animal (typically mammalian) origin that have been modified using genetic engineering techniques. The techniques are used to replace constant region and/or variable region framework sequences with juman sequences, while retaining the original antigen specificity of the antibody. Humanized and primatized® antibodies are commonly derived from rodent (e.g., mouse and hamster) antibodies with specificity for human antigens (e.g., human VCAM-1 or human VLA-4). By reshaping the donor antibody (the antibody from the animal to which the antigen was administered) to have sequences from the animal to which the antibody will be administered for therapeutic purposes, there will be a reduced host response in the animal upon administration of the antibody. Only the Fc regions or all but the complementarity determining regions (CDRs) can be replaced with acceptor domains, wherein the acceptor is the animal to whom the reshaped antibody is to be administered (e.g., mammals such as humans, domesticated animals, agricultural animals and the like).

Antibodies that bind to the α₄ subunit of VLA-4 which when administered to a patient in an effective amount treat inflammatory bowel disease, asthma, multiple sclerosis (MS), rheumatoid arthritis (RA), graft versus host disease (GVHD), host versus graft disease, and various spondyloarthropathies are preferred.

Typically, CDRs of a murine antibody are transplanted onto the corresponding regions in a human antibody, since it is the CDRs (ie., three in antibody heavy chains, three in light chains) that are the regions of the mouse antibody (or any other animal antibody), which bind to a specific antigen. Transplantation of CDRs is achieved by genetic engineering, whereby CDR DNA sequences are determined by cloning of murine heavy and light chain variable (V) region gene segments, and are then transferred to corresponding human V regions by site directed mutagenesis. In the final stage of the process, human constant region gene segments of the desired isotype (usually gamma I for CH and kappa for CL) are added and the humanized heavy and light chain genes are co-expressed in mammalian cells to produce soluble humanized antibody.

The transfer of these CDRs to a human antibody confers on this antibody the antigen binding properties of the original murine antibody. The six CDRs in the murine antibody are mounted structurally on a V region “framework” region. The reason that CDR-grafting is successful is that framework regions between mouse and human antibodies may have very similar 3-D structures with similar points of attachment for CDRS, such that CDRs can be interchanged. Such humanized antibody homologs may be prepared, as exemplified in, e.g., Jones et al., 1986, Nature 321: 522-5; Riechmann et al., 1988, Nature 332: 323-7; Queen et al., 1989, Proc. Nat. Acad. Sci. USA 86: 10029; and Orlandi et al., 1989, Proc. Nat. Acad. Sci. USA 86: 3833.

Nonetheless, certain amino acids within framework regions are thought to interact with CDRs and to influence overall antigen binding affinity. The direct transfer of CDRs from a murine antibody to produce a recombinant humanized antibody without any modifications of the human V region frameworks often results in a partial or complete loss of binding affinity. In several cases, it appears to be critical to alter residues in the framework regions of the acceptor antibody (e.g., human antibody) in order to obtain binding activity.

Queen et al., 1989 (supra) and WO 90/07861 (Protein Design Labs) have described the preparation of a humanized antibody that contains modified residues in the framework regions of the acceptor antibody by combining the CDRs of a murine MAb (anti-Tac) with human immunoglobulin framework and constant regions. One solution to solve the problem of the loss of binding affinity without any modifications of the human V region framework residues involves two key steps. First, the human V framework regions are chosen by computer analysts for optimal protein sequence homology to the V region framework of the original murine antibody. In the second step, the tertiary structure of the murine V region is modeled by computer in order to visualize framework amino acid residues that are likely to interact with the murine CDRs. These murine amino acid residues are then superimposed on the homologous human framework. For additional detail, see U.S. Pat. Nos. 5,693,762; 5,693,761; 5,585,089; and 5,530,101 (Protein Design Labs).

Certain α₄ subunit-containing integrin antagonists useful in the present invention include chimeric and humanized recombinant antibody homologs (i.e., intact immunoglobulins and portions thereof) with B epitope specificity that have been prepared and are described in U.S. Pat. No. 5,932,214 (MAb HP1/2). The starting material for the preparation of chimeric (mouse Variable-human Constant) and humanized anti-integrin antibody homologs may be a murine monoclonal anti-integrin antibody as previously described, a monoclonal anti-integrin antibody commercially available (e.g., HP2/1, Amae International, Inc., Westbrook, Me.). Other preferred humanized anti-VLA-4 antibody homologs are described by Athena Neurosciences, Inc. in PCT/US95/01219 (Jul. 27, 1995), U.S. Pat. Nos. 5,840,299 and 6,033,665. The content of the U.S. Pat. Nos. 5,932,214, 5,840,299 and 6,033,665 patents are incorporated by reference in their entirety herein for all purposes.

These humanized anti-VLA-4 antibodies comprise a humanized light chain and a humanized heavy chain. The humanized light chain comprises three complementarity determining regions (CDR1, CDR2 and CDR3) having amino acid sequences from the corresponding complementarity determining regions of a mouse 21.6 immunoglobulin light chain, and a variable region framework from a human kappa light chain variable region framework sequence except in at least position the amino acid position is occupied by the same amino acid present in the equivalent position of the mouse 21.6 immunoglobulin light chain variable region framework. The humanized heavy chain comprises three complementarity determining regions (CDR1, CDR2 and CDR3) having amino acid sequences from the corresponding complementarity determining regions of a mouse 21.6 immunoglobulin heavy chain, and a variable region framework from a human heavy chain variable region framework sequence except in at least one position the amino acid position is occupied by the same amino acid present in the equivalent position of the mouse 21.6 immunoglobulin heavy chain variable region framework. See, U.S. Pat. Nos. 5,840,299 and 6,033,665.

Fragments of an isolated α₄ integrin antagonist (e.g., fragments of antibody homologs described herein) can also be produced efficiently by recombinant methods, by proteolytic digestion, or by chemical synthesis using methods known to those of skill in the art. In recombinant methods, internal or terminal fragments of a polypeptide can be generated by removing one or more nucleotides from one end (for a terminal fragment) or both ends (for an internal fragment) of a DNA sequence which encodes for the isolated hedgehog polypeptide. Expression of the mutagenized DNA produces polypeptide fragments. Digestion with certain endonucleases can also generate DNAs, which encode an array of fragments. DNAs that encode fragments of a protein can also be generated by random shearing, restriction digestion, or a combination thereof. Protein fragments can be generated directly from intact proteins. Peptides can be cleaved specifically by proteolytic enzymes, including, but not limited to plasmin, thrombin, trypsin, chymotrypsin, or pepsin. Each of these enzymes is specific for the type of peptide bond it attacks. Trypsin catalyzes the hydrolysis of peptide bonds in which the carbonyl group is from a basic amino acid, usually arginine or lysine. Pepsin and chymotrypsin catalyze the hydrolysis of peptide bonds from aromatic amino acids, such as tryptophan, tyrosine, and phenylalanine. Alternative sets of cleaved protein fragments are generated by preventing cleavage at a site which is susceptible to a proteolytic enzyme. For instance, reaction of the ε-amino acid group of lysine with ethyltrifluorothioacetate in mildly basic solution yields blocked amino acid residues whose adjacent peptide bond is no longer susceptible to hydrolysis by trypsin. Proteins can be modified to create peptide linkages that are susceptible to proteolytic enzymes. For instance, alkylation of cysteine residues with β-haloethylamines yields peptide linkages that are hydrolyzed by trypsin (Lindley, 1956, Nature 178: 647). In addition, chemical reagents that cleave peptide chains at specific residues can be used. For example, cyanogen bromide cleaves peptides at methionine residues (Gross et al., 1961, J. Am. Chem. Soc. 83: 1510). Thus, by treating proteins with various combinations of modifiers, proteolytic enzymes and/or chemical reagents, the proteins may be divided into fragments of a desired length with no overlap of the fragments, or divided into overlapping fragments of a desired length.

6.2.1 Natalizumab and Related Humanized Antibodies

The invention provides for a method of using humanized immunoglobulins that specifically bind to a VLA-4 ligand either alone or in combination to diagnose and/or treat inflammatory bowel disease such as Crohns's disease, asthma, multiple sclerosis (MS), rheumatoid arthritis (RA), graft versus host disease (GVHD), host versus graft disease, and various spondyloarthropathies. One preferred antibody for use in such methods of treatment and in medicaments includes that described in U.S. Pat. No. 5,840,299 assigned to Elan Pharmaceuticals, which is herein incorporated in its entirety. Another aspect contemplates the use of fragments of these antibodies as assessed in vivo.

The humanized antibodies comprise a humanized light chain and a humanized heavy chain. In one aspect, the humanized light chain can comprise three complementarity determining regions (i.e., CDR1, CDR2 and CDR3) having amino acid sequences from the corresponding complementarity determining regions of a mouse 21-6 immunoglobulin light chain, and a variable region framework from a human kappa light chain variable region framework sequence except in at least one position selected from a first group consisting of positions L45, L49, L58 and L69, wherein the amino acid position is occupied by the same amino acid present in the equivalent position of the mouse 21.6 immunoglobulin light chain variable region framework.

The humanized heavy chain comprises three complementarity determining regions (i.e., CDR1, CDR2 and CDR3) having amino acid sequences from the corresponding complementarity determining regions of a mouse 21-6 immunoglobulin heavy chain, and a variable region framework from a human heavy chain variable region framework sequence except in at least one position selected from a group consisting of H27, H28, H29, H30, H44, H71, wherein the amino acid position is occupied by the same amino acid present in the equivalent position of the mouse 21-6 immunoglobulin heavy chain variable region framework. The immunoglobulins specifically bind to VLA-4 with an affinity having a lower limit of about 10⁷ M⁻¹ and an upper limit of about five times the affinity of the mouse 21-6 immunoglobulin.

Usually, the humanized light and heavy chain variable region frameworks are from RE1 and 21/28′CL variable region framework sequences respectively. When the humanized light chain variable region framework is from RE1, at least two framework amino acids are replaced. One amino acid is from the first group of positions described supra. The other amino acids are from a third group consisting of positions L104, L105 and L107. This position is occupied by the same amino acid present in the equivalent position of a kappa light chain from a human immunoglobulin other than RE1.

Some humanized immunoglobulins have a mature light chain variable region sequence designated La or Lb, or a mature heavy chain variable region sequence designated Ha, Hb or Hc. Preferred humanized immunoglobulins include those having a La light chain and an Ha, Hb or Hc heavy chain.

The humanized immunoglobulins have variable framework regions substantially from a human immunoglobulin (termed an acceptor immunoglobulin) and complementarity determining regions substantially from a mouse immunoglobulin termed mu MAb 21.6 (referred to as the donor immunoglobulin). The constant region(s), if present, are also substantially from a human immunoglobulin. The humanized antibodies exhibit a specific binding affinity for VLA-4 of at least 10⁷, 10⁸, 10⁹, or 10¹⁰ M⁻¹. Usually the upper limit of binding affinity of the humanized antibodies for VLA-4 is within a factor of three or five of that of mu MAb 21.6 (about 10⁹ M⁻¹). Often the lower limit of binding affinity is also within a factor of three or five of that of mu MAb 21.6.

Humanized antibodies can be produced as exemplified, for example, with the mouse MAb 21.6 monoclonal antibody. The starting material for production of humanized antibodies is mu MAb 21.6. The isolation and properties of this antibody are described in U.S. Pat. No. 6,033,655 (assigned to Elan Pharmaceuticals, Inc.), which is herein incorporated by reference in its entirety. Briefly, mu MAb 21.6 is specific for the α₄ subunit of VLA-4 and has been shown to inhibit human lymphocyte binding to tissue cultures of rat brain cells stimulated with tumor necrosis factor. From N-terminal to C-terminal, both light and heavy chains comprise the domains FR1, CDR1, FR2, CDR2, FR3, CDR3 and FR4. The assignment of amino acids to each domain is in accordance with the numbering convention of Kabat.

The next step involved selecting human antibodies to supply framework residues. The substitution of mouse CDRs into a human variable domain framework is most likely to result in retention of their correct spatial orientation if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4: 773 (1991); Kolbinger et al., Protein Engineering 6: 971 (1993).

Suitable human antibody sequences are identified by computer comparisons of the amino acid sequences of the mouse variable regions with the sequences of known human antibodies. The comparison is performed separately for heavy and light chains but the principles are similar for each. This comparison reveals that the mu 21.6 light chain shows greatest sequence identity to human light chains of subtype kappa 1; the mu 21.6 heavy chain shows greatest sequence identity to human heavy chains of subtype one, as defined by Kabat, supra. Thus, light and heavy human framework regions are usually derived from human antibodies of these subtypes, or from consensus sequences of such subtypes. The preferred light and heavy chain human variable regions showing greatest sequence identity to the corresponding regions from mu MAb 21.6 are from antibodies RE1 and 21/28′CL respectively.

Computer modeling can then be used to further enhance the humanized antibody's ability to bind to its cognate antigen. The unnatural juxtaposition of murine CDR regions with human variable framework region can result in unnatural conformational restraints, which, unless corrected by substitution of certain amino acid residues, lead to loss of binding affinity. The selection of amino acid residues for substitution is determined, in part, by computer modeling. Computer hardware and software for producing three-dimensional images of immunoglobulin molecules are widely available. In general, molecular models are produced starting from solved structures for immunoglobulin chains or domains thereof. The chains to be modeled are compared for amino acid sequence similarity with chains or domains of solved three dimensional structures, and the chains or domains showing the greatest sequence similarity is/are selected as starting points for construction of the molecular model. For example, for the light chain of mu MAb 21.6, the starting point for modeling the framework regions, CDR1 and CDR2 regions, was the human light chain RE1. For the CDR3 region, the starting point was the CDR3 region from the light chain of a different human antibody HyHEL-5. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

As noted supra, the humanized antibodies of the invention comprise variable framework regions substantially from a human immunoglobulin and complementarity determining regions substantially from a mouse immunoglobulin termed mu MAb 21.6. Having identified the complementarity determining regions (CDRs) of mu MAb 21.6 and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a HAMA response in humans. Amino acids are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

When an amino acid differs between a mu MAb 21.6 variable framework region and an equivalent human variable framework region, the human framework amino acid should usually be substituted by the equivalent mouse amino acid if it is reasonably expected that the amino acid:
(1) non-covalently binds antigen directly (e.g., amino acids at positions L49, L69 of mu MAb 21.6),
(2) is adjacent to a CDR region, is part of a CDR region under the alternative definition proposed by Chothia et al., supra, or otherwise interacts with a CDR region (e.g., is within about 3 Å of a CDR region) (e.g., amino acids at positions L45, L58, H27, H28, H29, H30 and H71 of mu MAb 21.6), or
(3) participates in the V_{L}-V_{H} interface (e.g., amino acids at position H44 of mu MAb 21.6).

Other candidates for substitution are acceptor human framework amino acids that are unusual for a human immunoglobulin at that position (e.g., amino acids at positions L104, L105 and L107 of mu MAb 21.6). These amino acids can be substituted with amino acids from the equivalent position of more typical human immunoglobulins. Alternatively, amino acids from equivalent positions in the mouse MAb 21.6 can be introduced into the human framework regions when such amino acids are typical of human immunoglobulin at the equivalent positions.

In general, substitution of all or most of the amino acids fulfilling the above criteria is desirable. Occasionally, however, there is some ambiguity about whether a particular amino acid meets the above criteria, and alternative variant immunoglobulins are produced, one of which has that particular substitution, the other of which does not. The humanized antibodies will usually contain a substitution of a human light chain framework residue with a corresponding mu MAb 21.6 residue in at least 1, 2 or 3, and more usually 4, of the following positions: L45, L49, L58 and L69. The humanized antibodies also usually contain a substitution of a human heavy chain framework residue in at least 1, 2, 3, 4, or 5, and sometimes 6, of the following positions: H27, H28, H29, H30, H44 and H71. Optionally, H36 may also be substituted. In preferred embodiments when the human light chain acceptor immunoglobulin is RE1, the light chain also contains substitutions in at least 1 or 2, and more usually 3, of the following positions: L104, L105 and L107. These positions are substituted with the amino acid from the equivalent position of a human immunoglobulin having a more typical amino acid residues.

Usually the CDR regions in humanized antibodies are substantially identical, and more usually, identical to the corresponding CDR regions in the mu MAb 21.6 antibody. Occasionally, however, it is desirable to change one of the residues in a CDR region. For example, Example 4 identifies an amino acid similarity between the mu MAb 21.6 CDR3 and the VCAM-1 ligand. This observation suggests that the binding affinity of humanized antibodies might be improved by redesigning the heavy chain CDR3 region to resemble VCAM-1 even more closely. Accordingly, one or more amino acids from the CDR3 domain can be substituted with amino acids from the VCAM-1 binding domain. Although not usually desirable, it is sometimes possible to make one or more conservative amino acid substitutions of CDR residues without appreciably affecting the binding affinity of the resulting humanized immunoglobulin.

Other than for the specific amino acid substitutions discussed above, the framework regions of humanized immunoglobulins are usually substantially identical, and more usually, identical to the framework regions of the human antibodies from which they were derived. Of course, many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin. However, in general, such substitutions are undesirable.

6.2.2 Production of Variable Regions

Having conceptually selected the CDR and framework components of humanized immunoglobulins, a variety of methods are available for producing such immunoglobulins. Because of the degeneracy of the code, a variety of nucleic acid sequences will encode each immunoglobulin amino acid sequence. The desired nucleic acid sequences can be produced by de novo solid-phase DNA synthesis or by PCR mutagenesis of an earlier prepared variant of the desired polynucleotide. Oligonucleotide-mediated mutagenesis is a preferred method for preparing substitution, deletion and insertion variants of target polypeptide DNA. See Adelman et al., DNA 2: 183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

6.2.3 Selection of Constant Region

The variable segments of humanized antibodies produced as described supra are typically linked to at least a portion of an immunoglobulin constant region (Fc), typically that of a human immunoglobulin. Human constant region DNA sequences can be isolated in accordance with well-known procedures from a variety of human cells, but preferably immortalized B-cells (see Kabat et al., supra, and WO 87/02671) (each of which is incorporated by reference in its entirety). Ordinarily, the antibody will contain both light chain and heavy chain constant regions. The heavy chain constant region usually includes CH₁, hinge, CH₂, CH₃, and CH₄ regions.

The humanized antibodies include antibodies having all types of constant regions, including IgM, IgG, IgD, IgA and IgE, and any isotype, including IgG1, IgG2, IgG3 and IgG4. When it is desired that the humanized antibody exhibit cytotoxic activity, the constant domain is usually a complement-fixing constant domain and the class is typically IgG₁. When such cytotoxic activity is not desirable, the constant domain may be of the IgG₂ class. The humanized antibody may comprise sequences from more than one class or isotype.

6.3 Other Anti-VLA-4 Antibodies

Other anti-VLA-4 antibodies include but are not limited to HP1/2, HP-2/1, HP2/4, L25, and P4C2. These antibodies may also be administered in an effective amount to diagnose and/or treat imflammatory bowel conditions as one skilled in the art as discussed herein and as generally known in the art would readily appreciate.

Frequently, monoclonal antibodies created in mice are later humanized to avoid the human anti-mouse antibody (HAMA) immune response in a human subject injected with a mouse antibody. This occurs by CDR grafting or reshaping. Thus, typically the antibodies are first mouse monoclonal antibodies that through CDR grafting or reshaping become humanized, as discussed above for the 21.6 antibody.

Specifically, the humanized antibodies have specificity for VLA-4 and have the ability to diagnose and/or treat imflammatory bowel conditions. These antibodies are derived from sources (e.g., mouse typically) that at least one or more of the complementarity determining regions (CDRs) of the variable domains are derived from a donor non-human anti-VLA-4 antibody, and in which there may or may not have been minimal alteration of the acceptor antibody heavy and/or light variable framework region in order to retain donor antibody binding specificity. Preferably, the antigen binding regions of the CDR-grafted heavy chain variable domain comprise the CDRs corresponding to positions 31-35 (CDR1), 50-65 (CDR2) and 95-102 (CDR3). In a preferred embodiment, the heavy chain further includes non-human residues at framework positions 27-30 (Kabat numbering). The heavy chain can further include non-human residues at framework position 75 (Kabat numbering). The heavy chain can further include non-human residues at framework position(s) 77-79 or 66-67 and 69-71 or 84-85 or 38 and 40 or 24 (Kabat numbering). Preferably, the antigen binding regions of the CDR-grafted light chain variable domain comprise CDRs corresponding to positions 24-34 (CDR1), 50-56 (CDR2) and 89-97 (CDR3). In a preferred embodiment, the light chain further includes non-human residues at framework positions 60 and 67 (Kabat numbering). These residue designations are numbered according to the Kabat numbering (Kabat et al., 5^{th} ed. 4 vol. SEQUENCES OF PROTEINS OF IMMUNOLOGICAL INTEREST, U.S. Department of Health Human Services, NIH, USA (1991)).

Synthesis and Humanization of Mouse Antibody HP1/2. HP1/2 is another antibody that is directed against VLA-4. The method of preparing a humanized version of this antibody for use in human subjects is described herein and is further described in U.S. Pat. No. 6,602,503 assigned to Biogen, Inc., and hereby incorporated by reference in its entirety. The sequences of the humanized antibodies are provided as follows. The HP1/2 V_{H} DNA sequence and its translated amino acid sequence are:

```
    5′-gtc aaa ctg cag cag tct ggg gca gag ctt gtg aag cca ggg gcc tca
48
      N-Val Lys Leu Gln Gln Ser Gly Ala Glu Leu Val Lys Pro Gly Ala Ser
          1 5 10 15
        gtc aag ttg ttc tgc aca gct tct ggc ttc aac att aaa gac acc tat
96
        Val Lys Leu Phe Cys Thr Ala Ser Gly Phe Asn Ile Lys Asp Thr Tyr
                                20 25 30
        atg cac tgg gtg aag cag agg cct caa cag ggc ctg gag tgg att gga
144
        Met His Trp Val Lys Gln Arg Pro Gln Gln Gly Leu Glu Trp Ile Gly
                        35 40 45
        agg att gat cct gcg agt ggc gat act aaa tat gac ccg aag ttc cag
192
        Arg Ile Asp Pro Ala Ser Gly Asp Thr Lys Tyr Asp Pro Lys Phe Gln
                  50 55 60
        gtc aag gcc act att aca gcg gac acg tcc tcc aac aca gcc tgg ctg
240
        Val Lys Ala Thr Ile Thr Ala Asp Thr Ser Ser Asn Thr Ala Trp Leu
          65 70 75 80
        cag ctc agc agc ctg aca tct gag gac act gcc gtc tac tac tgt gca
288
        Gln Leu Ser Ser Leu Thr Ser Glu Asp Thr Ala Val Tyr Tyr Cys Ala
                                          85 90 95
        gac gga atg tgg gta tca acg gga tat gct ctg gac ttc tgg ggc caa
336
        Asp Gly Met Trp Val Ser Thr Gly Tyr Ala Leu Asp Phe Trp Gly Gln
                                100 105 110
        ggg acc acg gtc acc gtc tcc tca-3′
360
        Gly Thr Thr Val Thr Val Ser Ser-C
                        115 120
```

A comparison between HP1/2 V_{H} the two sequences and a consensus sequence of family IIC revealed that the only unusual residues are at amino acid positions 80, 98 and 121 (i.e., 79, 94 and 121 in Kabat numbering). Although Tyr-80 is invariant in subgroup IIC other sequenced murine V_{H} regions have other aromatic amino acids at this position, although none have Trp. The majority of human and murine V_{H}s have an arginine residue at Kabat position 94. The presence of Asp-94 in HP1/2 V_{H} is extremely rare; there is only one reported example of a negatively charged residue at this position. Proline at Kabat position 113 is also unusual but is unlikely to be important in the conformation of the CDRs because of its distance from them. The amino acids making up CDR1 have been found in three other sequenced murine V_{H} regions. However, CDR2 and CDR3 are unique to HP1/2 and are not found in any other reported murine V_{H}.

The HP1/2 V_{K} DNA sequence and its translated amino acid sequence are as follows:

```
  5′-agt att gtg atg acc cag act ccc aaa ttc ctg ctt gtt tca gca gga
48
    N-Ser Ile Val Met Thr Gln Thr Pro Lys Phe Leu Leu Val Ser Ala Gly
          1 5 10 15
        gac agg gtt acc ata acc tgc aag gcc agt cag agt gtg act aat gat
96
        Asp Arg Val Thr Ile Thr Cys Lys Ala Ser Gln Ser Val Thr Asn Asp
                                  20 25 30
        gta gct tgg tac caa cag aag cca ggg cag tct cct aaa ctg ctg ata
144
        Val Ala Trp Tyr Gln Gln Lys Pro Gly Gln Ser Pro Lys Leu Leu Ile
                        35 40 45
        tat tat gca tcc aat cgc tac act gga gtc cct gat cgc ttc act ggc
192
        Tyr Tyr Ala Ser Asn Arg Tyr Thr Gly Val Pro Asp Arg Phe Thr Gly
                  50 55 60
        agt gga tat ggg acg gat ttc act ttc acc atc agc act gtg cag gct
240
        Ser Gly Tyr Gly Thr Asp Phe Thr Phe Thr Ile Ser Thr Val Gln Ala
          65 70 75 80
        gaa gac ctg gca gtt tat ttc tgt cag cag gat tat agc tct ccg tac
288
        Glu Asp Leu Ala Val Tyr Phe Cys Gln Gln Asp Tyr Ser Ser Pro Tyr
                                          85 90 95
        acg ttc gga ggg ggg acc aag ctg gag atc-3′
318
        Thr Phe Gly Gly Gly Thr Lys Leu Glu Ile-C
                                100 105
```

HP1/2 V_{K} is a member of Kabat family V (Kabat et al., 5^{th} ed., 4 vol., SEQUENCES OF PROTEINS OF IMMUNOLOGICAL INTEREST, U.S. Department of Health Human Services (1991)) and has no unusual residues. The amino acids of CDR1 and CDR3 are unique. The amino acids making up CDR2 have been reported in one other murine V_{K}.

Design of a CDR-grafted Anti-VLA-4 Antibody. To design a CDR-grafted anti-VLA-4 antibody, it was necessary to determine which residues of murine HP1/2 comprise the CDRs of the light and heavy chains. Three regions of hypervariability amid the less variable framework sequences are found on both light and heavy chains (Wu and Kabat, J. Exp. Med. 132: 211-250 (1970); Kabat et al. (1991)). In most cases these hypervariable regions correspond to, but may extend beyond, the CDR. CDRs of murine HP1/2 were elucidated in accordance with Kabat et al. (1991) by alignment with other V_{H} and V_{K} sequences. The CDRs of murine HP1/2 V_{H} were identified and correspond to the residues identified in the humanized V_{H} sequences as follows:

| | |
|---|---|
| CDR1 | AA₃₁-AA₃₅ |
| CDR2 | AA₅₀-AA₆₆ |
| CDR3 | AA₉₉-AA₁₁₀ |

These correspond to AA₃₁-AA₃₅, AA₅₀-AA₆₅, and AA₉₅-AA₁₀₂, respectively, in Kabat numbering. The CDRs of murine HP1/2 V_{K} were identified and correspond to the residues identified in the humanized V_{K} sequences as follows:

| | |
|---|---|
| CDR1 | AA₂₄-AA₃₄ |
| CDR2 | AA₅₀-AA₅₆ |
| CDR3 | AA₈₉-AA₉₇ |

These correspond to the same numbered amino acids in Kabat numbering. Thus, only the boundaries of the V_{K}, but not V_{H}, CDRs corresponded to the Kabat CDR residues. The human frameworks chosen to accept the HP1/2 (donor) CDRs were NEWM and RE1 for the heavy and light chains, respectively. The NEWM and the RE1 sequences have been published in Kabat et al. (1991).

The DNA and corresponding amino acid sequence of the humanized heavy chain variable region of the humanized HP1/2 antibody is:

```
  5′-atg gac tgg acc tgg agg gtc ttc tgc ttg ctg gct gta gca cca ggt
48
    N-Met Asp Trp Thr Trp Arg Val Phe Cys Leu Leu Ala Val Ala Pro Gly
          1 5 10 15
        gcc cac tcc cag gtc caa ctg cag gag tcc ggt gct gaa gtt gtt aaa
96
        Ala His Ser Gln Val Gln Leu Gln Glu Ser Gly Ala Glu Val Val Lys
                                  20 25 30
        ccg ggt tcc tcc gtt aaa ctg tcc tgc aaa gct tcc ggt ttc aac atc
144
        Pro Gly Ser Ser Val Lys Leu Ser Cys Lys Ala Ser Gly Phe Asn Ile
                        35 40 45
        aaa gac acc tac atg cac tgg gtt aaa cag cgt ccg ggt cag ggt ctg
192
        Lys Asp Thr Tyr Met His Trp Val Lys Gln Arg Pro Gly Gln Gly Leu
                  50 55 60
        gaa tgg atc ggt cgt atc gac ccg gct tcc ggt gac acc aaa tac gac
240
        Glu Trp Ile Gly Arg Ile Asp Pro Ala Ser Gly Asp Thr Lys Tyr Asp
          65 70 75 80
        ccg aaa ttc cag gtt aaa gct acc atc acc gct gac gaa tcc acc tcc
288
        Pro Lys Phe Gln Val Lys Ala Thr Ile Thr Ala Asp Glu Ser Thr Ser
                                          85 90 95
        acc gct tac ctg gaa ctg tcc tcc ctg cgt tcc gaa gac acc gct gtt
336
        Thr Ala Tyr Leu Glu Leu Ser Ser Leu Arg Ser Glu Asp Thr Ala Val
                                100 105 110
        tac tac tgc gct gac ggt atg tgg gtt tcc acc ggt tac gct ctg gac
384
        Tyr Tyr Cys Ala Asp Gly Met Trp Val Ser Thr Gly Tyr Ala Leu Asp
                        115 120 125
        ttc tgg ggt cag ggt acc acg gtc acc gtc tcc tca ggt gag tcc-3′
429
        Phe Trp Gly Gln Gly Thr Thr Val Thr Val Ser Ser Gly Glu Ser-C
                130 135 140
```

The DNA and corresponding amino acid sequence of the humanized light chain variable region of the humanized HP1/2 antibody:

```
  5′-atg ggt tgg tcc tgc atc atc ctg ttc ctg gtt gct acc gct acc ggt
48
    N-Met Gly Trp Ser Cys Ile Ile Leu Phe Leu Val Ala Thr Ala Thr Gly
          1 5 10 15
        gtt cac tcc atc gtt atg acc cag tcc ccg gac tcc ctg gct gtt tcc
96
        Val His Ser Ile Val Met Thr Gln Ser Pro Asp Ser Leu Ala Val Ser
                                  20 25 30
        ctg ggt gaa cgt gtt acc atc aac tgc aaa gct tcc cag tcc gtt acc
144
        Leu Gly Glu Arg Val Thr Ile Asn Cys Lys Ala Ser Gln Ser Val Thr
                          35 40 45
        aac gac gtt gct tgg tac cag cag aaa ccg ggt cag tcc ccg aaa ctg
192
        Asn Asp Val Ala Trp Tyr Gln Gln Lys Pro Gly Gln Ser Pro Lys Leu
                      50 55 60
        ctg atc tac tac gct tcc aac cgt tac acc ggt gtt ccg gac cgt ttc
240
        Leu Ile Tyr Tyr Ala Ser Asn Arg Tyr Thr Gly Val Pro Asp Arg Phe
          65 70 75 80
        tcc ggt tcc ggt tac ggt acc gac ttc acc ttc acc atc tcc tcc gtt
288
        Ser Gly Ser Gly Tyr Gly Thr Asp Phe Thr Phe Thr Ile Ser Ser Val
                                          85 90 95
        cag gct gaa gac gtt gct gtt tac tac tgc cag cag gac taG tcc tcc
336
        Gln Ala Glu Asp Val Ala Val Tyr Tyr Cys Gln Gln Asp Tyr Ser Ser
                                100 105 110
        ccg tac acc ttc ggt ggt ggt acc aaa ctg gag atc taa ggatcctc-3′
383
        Pro Tyr Thr Phe Gly Gly Gly Thr Lys Leu Glu Ile-C
                        115 120
```

In addition to the above humanized HP1/2 antibody light and heavy chains, other acceptor heavy and light chains regions can also be utilized for insertion of the donor HP1/2 regions. All the following constructs contain Ser-75 (Kabat numbering). The STAW construct further contains Gln to Thr at position 77, Phe to Ala at position 78, and Ser to Trp at position 79 (Kabat numbering). The V_{H} DNA sequence and its translated amino acid sequence are set forth below:

```
  5′-atg gac tgg acc tgg agg gtc ttc tgc ttg ctg gct gta gca cca ggt
48
    N-Met Asp Trp Thr Trp Arg Val Phe Cys Leu Leu Ala Val Ala Pro Gly
          1 5 10 15
        gcc cac tcc cag gtc caa ctg cag gag agc ggt cca ggt ctt gtg aga
96
        Ala His Ser Gln Val Gln Leu Gln Glu Ser Gly Pro Gly Leu Val Arg
                                  20 25 30
        cct agc cag acc ctg agc ctg acc tgc acc gtg tct ggc ttc aac att
144
        Pro Ser Gln Thr Leu Ser Leu Thr Cys Thr Val Ser Gly Phe Asn Ile
                          35 40 45
        aaa gac acc tat atg cac tgg gtg aga cag cca cct gga cga ggt ctt
192
        Lys Asp Thr Tyr Met His Trp Val Arg Gln Pro Pro Gly Arg Gly Leu
                      50 55 60
        gag tgg att gga agg att gat cct gcg agt ggc gat act aaa tat gac
240
        Glu Trp Ile Gly Arg Ile Asp Pro Ala Ser Gly Asp Thr Lys Tyr Asp
          65 70 75 80
        ccg aag ttc cag gtc aga gtg aca atg ctg gta gac acc agc agc aac
288
        Pro Lys Phe Gln Val Arg Val Thr Met Leu Val Asp Thr Ser Ser Asn
                                          85 90 95
        aca gcc tgg ctg aga ctc agc agc gtg aca gcc gcc gac acc gcg gtC
336
        Thr Ala Trp Leu Arg Leu Ser Ser Val Thr Ala Ala Asp Thr Ala Val
                                100 105 110
        tat tat tgt gca gac gga atg tgg gta tca acg gga tat gct ctg gac
384
        Tyr Tyr Cys Ala Asp Gly Met Trp Val Ser Thr Gly Tyr Ala Leu Asp
                        115 120 125
        ttc tgg ggc caa ggg acc acg gtc acc gtc tcc tca ggt gag tcc-3′
429
        Phe Trp Gly Gln Gly Thr Thr Val Thr Val Ser Ser Gly Glu Ser-C
                130 135 140
```

The KAITAS construct contains the additional changes of Arg to Lys (position 66), Val to Ala (position 67), Met to Ile (position 69), Leu to Thr (position 70) and Val to Ala (position 71) (Kabat numbering. The KAITAS V_{H} DNA sequence and its translated amino acid sequence are set forth below:

```
  5′-atg gac tgg acc tgg agg gtc ttc tgc ttg ctg gct gta gca cca ggt
48
    N-Met Asp Trp Thr Trp Arg Val Phe Cys Leu Leu Ala Val Ala Pro Gly
          1 5 10 15
        gcc cac tcc cag gtc caa ctg cag gag agc ggt cca ggt ctt gtg aga
96
        Ala His Ser Gln Val Gln Leu Gln Glu Ser Gly Pro Gly Leu Val Arg
                                  20 25 30
        cct agc cag acc ctg agc ctg acc tgc acc gtg tct ggc ttc aac att
144
        Pro Ser Gln Thr Leu Ser Leu Thr Cys Thr Val Ser Gly Phe Asn Ile
                          35 40 45
        aaa gac acc tat atg cac tgg gtg aga cag cca cct gga cga ggt ctt
192
        Lys Asp Thr Tyr Met His Trp Val Arg Gln Pro Pro Gly Arg Gly Leu
                      50 55 60
        gag tgg att gga agg att gat cct gcg agt ggc gat act aaa tat gac
240
        Glu Trp Ile Gly Arg Ile Asp Pro Ala Ser Gly Asp Thr Lys Tyr Asp
          65 70 75 80
        ccg aag ttc cag gtc aaa gcg aca att acg gca gac acc agc agc aac
288
        Pro Lys Phe Gln Val Lys Ala Thr Ile Thr Ala Asp Thr Ser Ser Asn
                                          85 90 95
        cag ttc agc ctg aga ctc agc agc gtg aca gcc gcc gac acc gcg gtc
336
        Gln Phe Ser Leu Arg Leu Ser Ser Val Thr Ala Ala Asp Thr Ala Val
                                100 105 110
        tat tat tgt gca gac gga atg tgg gta tca acg gga tat gct ctg gac
384
        Tyr Tyr Cys Ala Asp Gly Met Trp Val Ser Thr Gly Tyr Ala Leu Asp
                        115 120 125
        ttc tgg ggc caa ggg acc acg gtc acc gtc tcc tca ggt gag tcc-3′
429
        Phe Trp Gly Gln Gly Thr Thr Val Thr Val Ser Ser Gly Glu Ser-C
                130 135 140
```

The SSE construct comprises the additional changes of Ala to Ser (position 84) and Ala to Glu (position 85) (Kabat numbering). The SSE V_{H} DNA sequence and its translated amino acid sequence are set forth below:

```
  5′-cag gtc caa ctg cag gag agc ggt cca ggt ctt gtg aga cct agc cag
48
    N-Gln Val Gln Leu Gln Glu Ser Gly Pro Gly Leu Val Arg Pro Ser Gln
          1 5 10 15
        acc ctg agc ctg acc tgc acc gtg tct ggc ttc aac att aaa gac acc
96
        Thr Leu Ser Leu Thr Cys Thr Val Ser Gly Phe Asn Ile Lys Asp Thr
                                  20 25 30
        tat atg cac tgg gtg aga cag cca cct gga cga ggt ctt gag tgg att
144
        Tyr Met His Trp Val Arg Gln Pro Pro Gly Arg Gly Leu Glu Trp Ile
                          35 40 45
        gga agg att gat cct gcg agt ggc gat act aaa tat gac ccg aag ttc
192
        Gly Arg Ile Asp Pro Ala Ser Gly Asp Thr Lys Tyr Asp Pro Lys Phe
                      50 55 60
        cag gtc aga gtg aca atg ctg gta gac acc agc agc aac cag ttc agc
240
        Gln Val Arg Val Thr Met Leu Val Asp Thr Ser Ser Asn Gln Phe Ser
          65 70 75 80
        ctg aga ctc agc agc gtg aca tct gag gac acc gcg gtc tat tat tgt
288
        Leu Arg Leu Ser Ser Val Thr Ser Glu Asp Thr Ala Val Tyr Tyr Cys
                                          85 90 95
        gca gac gga atg tgg gta tca acg gga tat gct ctg gac ttc tgg ggc
336
        Ala Asp Gly Met Trp Val Ser Thr Gly Tyr Ala Leu Asp Phe Trp Gly
                                100 105 110
        caa ggg acc acg gtc acc gtc tcc tca ggt gag tcc-3′
372
        Gln Gly Thr Thr Val Thr Val Ser Ser Gly Glu Ser-C
                        115 120
```

The KRS construct comprises the additional changes of Arg to Lys (position 38) and Pro to Arg (position 40) (Kabat numbering). The KRS V_{H} DNA sequence and its translated amino acid sequence are set forth below:

```
  5′-atg gac tgg acc tgg agg gtc ttc tgc ttg ctg gct gta gca cca ggt
48
    N-Met Asp Trp Thr Trp Arg Val Phe Cys Leu Leu Ala Val Ala Pro Gly
          1 5 10 15
        gcc cac tcc cag gtc caa ctg cag gag agc ggt cca ggt ctt gtg aga
96
        Ala His Ser Gln Val Gln Leu Gln Glu Ser Gly Pro Gly Leu Val Arg
                                  20 25 30
        cct agc cag acc ctg agc ctg acc tgc acc gtg tct ggc ttc aac att
144
        Pro Ser Gln Thr Leu Ser Leu Thr Cys Thr Val Ser Gly Phe Asn Ile
                          35 40 45
        aaa gac acc tat atg cac tgg gtg aaa cag cga cct gga cga ggt ctt
192
        Lys Asp Thr Tyr Met His Trp Val Lys Gln Arg Pro Gly Arg Gly Leu
                      50 55 60
        gag tgg att gga agg att gat cct gcg agt ggc gat act aaa tat gac
240
        Glu Trp Ile Gly Arg Ile Asp Pro Ala Ser Gly Asp Thr Lys Tyr Asp
          65 70 75 80
        ccg aag ttc cag gtc aga gtg aca atg ctg gta gac acc agc agc aac
288
        Pro Lys Phe Gln Val Arg Val Thr Met Leu Val Asp Thr Ser Ser Asn
                                          85 90 95
        cag ttc agc ctg aga ctc agc agc gtg aca gcc gcc gac acc gcg gtc
336
        Gln Phe Ser Leu Arg Leu Ser Ser Val Thr Ala Ala Asp Thr Ala Val
                                100 105 110
        tat tat tgt gca gac gga atg tgg gta tca acg gga tat gct ctg gac
384
        Tyr Tyr Cys Ala Asp Gly Met Trp Val Ser Thr Gly Tyr Ala Leu Asp
                        115 120 125
        ttc tgg ggc caa ggg acc acg gtc acc gtc tcc tca ggt gag tcc-3′
429
        Phe Trp Gly Gln Gly Thr Thr Val Thr Val Ser Ser Gly Glu Ser-C
                130 135 140
```

The AS construct comprises the change Val to Ala at position 24 (Kabat numbering). The AS V_{H} DNA sequence and its translated amino acid sequence are:

```
  5′-atg gac tgg acc tgg agg gtc ttc tgc ttg ctg gct gta gca cca ggt
48
    N-Met Asp Trp Thr Trp Arg Val Phe Cys Leu Leu Ala Val Ala Pro Gly
          1 5 10 15
        gcc cac tcc cag gtc caa ctg cag gag agc ggt cca ggt ctt gtg aga
96
        Ala His Ser Gln Val Gln Leu Gln Glu Ser Gly Pro Gly Leu Val Arg
                                  20 25 30
        cct agc cag acc ctg agc ctg acc tgc acc gcg tct ggc ttc aac att
144
        Pro Ser Gln Thr Leu Ser Leu Thr Cys Thr Ala Ser Gly Phe Asn Ile
                          35 40 45
        aaa gac acc tat atg cac tgg gtg aga cag cca cct gga cga ggt ctt
192
        Lys Asp Thr Tyr Met His Trp Val Arg Gln Pro Pro Gly Arg Gly Leu
                      50 55 60
        gag tgg att gga agg att gat cct gcg agt ggc gat act aaa tat gac
240
        Glu Trp Ile Gly Arg Ile Asp Pro Ala Ser Gly Asp Thr Lys Tyr Asp
          65 70 75 80
        ccg aag ttc cag gtc aga gtg aca atg ctg gta gac acc agc agc aac
288
        Pro Lys Phe Gln Val Arg Val Thr Met Leu Val Asp Thr Ser Ser Asn
                                          85 90 95
        cag ttc agc ctg aga ctc agc agc gtg aca gcc gcc gac acc gcg gtc
336
        Gln Phe Ser Leu Arg Leu Ser Ser Val Thr Ala Ala Asp Thr Ala Val
                                100 105 110
        tat tat tgt gca gac gga atg tgg gta tca acg gga tat gct ctg gac
384
        Tyr Tyr Cys Ala Asp Gly Met Trp Val Ser Thr Gly Tyr Ala Leu Asp
                        115 120 125
        ttc tgg ggc caa ggg acc acg gtc acc gtc tcc tca ggt gag tcc-3′
429
        Phe Trp Gly Gln Gly Thr Thr Val Thr Val Ser Ser Gly Glu Ser-C
                130 135 140
```

The humanized light chain generally requires few, if any, modifications. However, in the preparation of humanized anti-VLA-4 antibodies, several empirical changes did improve the immunological activity of the antibody towards its ligand. For example, the humanized heavy chain with the Ser mutation with the murine light chain was about 2.5 fold lower potency than murine HP1/2. The same humanized heavy chain with a humanized light chain was about 4-fold lower potency.

A humanized V_{K} construct (VK1) comprises a Ser to Asp substitution at position 60, and a Ser for a Tyr at position 67. The DNA sequence and its translated amino acid sequence are set forth below:

```
  5′-atg ggt tgg tcc tgc aic atc ctg ttc ctg gtt gct acc gct acc ggt
48
    N-Met Gly Trp Ser Cys Ile Ile Leu Phe Leu Val Ala Thr Ala Thr Gly
          1 5 10 15
        gtt cac tcc gac atc cag ctg acc cag agc cca agc agc ctg agc gcc
96
        Val His Ser Asp Ile Gln Leu Thr Gln Ser Pro Ser Ser Leu Ser Ala
                                  20 25 30
        agc gtg ggt gac aga gtg acc atc acc tgt aag gcc agt cag agt gtg
144
        Ser Val Gly Asp Arg Val Thr Ile Thr Cys Lys Ala Ser Gln Ser Val
                          35 40 45
        act aat gat gta gct tgg tac cag cag aag cca ggt aag gct cca aag
192
        Thr Asn Asp Val Ala Trp Tyr Gln Gln Lys Pro Gly Lys Ala Pro Lys
                      50 55 60
        ctg ctg atc tac tat gca tcc aat cgc tac act ggt gtg cca agc aga
240
        Leu Leu Ile Tyr Tyr Ala Ser Asn Arg Tyr Thr Gly Val Pro Ser Arg
          65 70 75 80
        ttc agc ggt agc ggt agc ggt acc gac ttc acc ttc acc atc agc agc
288
        Phe Ser Gly Ser Gly Ser Gly Thr Asp Phe Thr Phe Thr Ile Ser Ser
                                          85 90 95
        ctc cag cca gag gac atc gcc acc tac tac tgc cag cag gat tat agc
336
        Leu Gln Pro Glu Asp Ile Ala Thr Tyr Tyr Cys Gln Gln Asp Tyr Ser
                                100 105 110
        tct ccg tac acg ttc ggc caa ggg acc aag gtg gaa atc aaa cgt aag tg-3′
386
        Ser Pro Tyr Thr Phe Gly Gln Gly Thr Lys Val Glu Ile Lys Arg Lys-C
                        115 120 125
```

Another V_{K} construct (i.e., VK2) has the DQMDY sequences of the original RE1 framework restored. The DNA and corresponding amino acid sequence are provided below:

```
  5′-atg ggt tgg tcc tgc atc atc ctg ttc ctg gtt gct acc gct acc ggt
48
    N-Met Gly Trp Ser Cys Ile Ile Leu Phe Leu Val Ala Thr Ala Thr Gly
          1 5 10 15
        gtc cac tcc agc atc gtg atg acc cag agc cca agc agc ctg agc gcc
96
        Val His Ser Ser Ile Val Met Thr Gln Ser Pro Ser Ser Leu Ser Ala
                                  20 25 30
        agc gtg ggt gac aga gtg acc atc acc tgt aag gcc agt cag agt gtg
144
        Ser Val Gly Asp Arg Val Thr Ile Thr Cys Lys Ala Ser Gln Ser Val
                        35 40 45
        act aat gat gta gct tgg tac cag cag aag cca ggt aag gct cca aag
192
        Thr Asn Asp Val Ala Trp Tyr Gln Gln Lys Pro Gly Lys Ala Pro Lys
                  50 55 60
        ctg ctg atc tac tat gca tcc aat cgc tac act ggt gtg cca gat aga
240
        Leu Leu Ile Tyr Tyr Ala Ser Asn Arg Tyr Thr Gly Val Pro Asp Arg
          65 70 75 80
        ttc agc ggt agc ggt tat ggt acc gac ttc acc ttc acc atc agc agc
288
        Phe Ser Gly Ser Gly Tyr Gly Thr Asp Phe Thr Phe Thr Ile Ser Ser
                                          85 90 95
        ctc cag cca gag gac atc gcc acc tac tac tgc cag cag gat tat agc
336
        Leu Gln Pro Glu Asp Ile Ala Thr Tyr Tyr Cys Gln Gln Asp Tyr Ser
                                100 105 110
        tct ccg tac acg ttc ggc caa ggg acc aag gtg gaa atc aaa cgt aag tg-3′
386
        Ser Pro Tyr Thr Phe Gly Gln Gly Thr Lys Val Glu Ile Lys Arg Lys-C
                        115 120 125
```

A third V_{K} construct is VK3 has SVM versus DQM in the amino terminus and two other residue changes. The DNA and corresponding amino acid sequence are:

```
  5′-atg ggt tgg tcc tgc atc atc ctg ttc ctg gtt gct acc gct acc ggt
48
    N-Met Gly Trp Ser Cys Ile Ile Leu Phe Leu Val Ala Thr Ala Thr Gly
          1 5 10 15
        gtc cac tcc gac atc cag atg acc cag agc cca agc agc ctg agc gcc
96
        Val His Ser Asp Ile Gln Met Thr Gln Ser Pro Ser Ser Leu Ser Ala
                                  20 25 30
        agc gtg ggt gac aga gtg acc atc acc tgt aag gcc agt cag agt gtg
144
        Ser Val Gly Asp Arg Val Thr Ile Thr Cys Lys Ala Ser Gln Ser Val
                        35 40 45
        act aat gat gta gct tgg tac cag cag aag cca ggt aag gct cca aag
192
        Thr Asn Asp Val Ala Trp Tyr Gln Gln Lys Pro Gly Lys Ala Pro Lys
                  50 55 60
        ctg ctg atc tac tat gca tcc aat cgc tac act ggt gtg cca gat aga
240
        Leu Leu Ile Tyr Tyr Ala Ser Asn Arg Tyr Thr Gly Val Pro Asp Arg
          65 70 75 80
        ttc agc ggt agc ggt tat ggt acc gac ttc acc ttc acc atc agc agc
288
        Phe Ser Gly Ser Gly Tyr Gly Thr Asp Phe Thr Phe Thr Ile Ser Ser
                                          85 90 95
        ctc cag cca gag gac atc gcc acc tac tac tgc cag cag gat tat agc
336
        Leu Gln Pro Glu Asp Ile Ala Thr Tyr Tyr Cys Gln Gln Asp Tyr Ser
                                100 105 110
        tct ccg tac acg ttc ggc caa ggg acc aag gtg gaa atc aaa cgt aag tg-3′
386
        Ser Pro Tyr Thr Phe Gly Gln Gly Thr Lys Val Glu Ile Lys Arg Lys-C
                        115 120 125
```

Details regarding how each of these light and heavy chain sequences were prepared are provided in U.S. Pat. No. 6,602,503, which is hereby incorporated by reference in its entirety for all puposes. Various combinations of the above light and heavy chains can be prepared based on computer modeling as known in the art.

Additional antibodies that recognize and bind to α₄ integrin are known in the art. These include but are not limited to GG5/3 (Keszthelyi et al., Neurology 47(4): 1053-1059 (1996)), FW3-218-1 (ATCC No.: HB-261; an IgG2b antibody against sheep α₄ integrin), and R¹-2 (ATCC No.: HB-227; IgG2b antibody developed in Rattus norvegicus). Whether the antibodies are developed in mouse or other animals, each of the sequences can be genetically engineered such that they are humanized based on what is known in the art and with the aid of computer modeling. The anti-α₄ integrin humanized antibodies can then be assessed for their ability to diagnose and/or treat imflammatory bowel conditions on the in vitro and in vivo assays disclosed herein.

6.4 Antibody Fragments

Also contemplated for use in diagnosing and/or treating imflammatory bowel conditions are antibody fragments of antibodies that bind to anti-α₄ or VCAM-1 such that they inhibit VLA-4 and VCAM-1 interaction. Antibody fragments include Fab, F(ab′)₂, scFv and Fv fragments which can be used in the compositions disclosed herein.

The term “Fab fragment” as used herein refers to a partial antibody molecule containing a single antigen-binding region, which consists of a portion of both the heavy and light chains of the molecule.

The term “F(ab′)₂ fragment” as used herein refers to a partial antibody molecule containing both antigen binding regions, and which consists of the light chains and a portion of the heavy chains of the molecule.

The term “Fv fragment” as used herein refers to the portion of the antibody molecule involved in antigen recognition and binding.

The term “scFv” as used herein refers to single chain Fv (scFv) fragments. These scFv fragments are recombinant antibody derivatives that consist only of the variable domains of antibody heavy and light chains connected by a flexible linker. scFv antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, 269-315 (Rosenburg and Moore eds., Springer-Verlag, New York 1994).

Also included in antibody fragments are diabodies. The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., 1993 Proc. Natl. Acad. Sci. USA 90: 6444-8.

Antibody fragments also include linear antibodies. The expression “linear antibodies” when used throughout this application refers to the antibodies described in, e.g., Zapata et al., 1995 Protein Eng. 8(10): 1057-62. Briefly, these antibodies comprise a pair of tandem Fd segments (V_{H}-C_{H}1-V_{H}-C_{H}1), which form a pair of antigen binding regions. Linear antibodies can be bispecific or monospecific.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen.

Several mouse anti-VLA-4 monoclonal antibodies have been previously described. See, e.g., U.S. Pat. Nos. 6,602,503, 6,033,665, and 5,840,299, as further discussed herein and which are herein incorporated by reference in their entirety; Sanchez-Madrid et al., 1986, Eur. J. Immunol. 16: 1343-9; Hemler et al., 1987, J. Biol. Chem. 262: 11478-85; Pulido et al., 1991, J. Biol. Chem., 266: 10241-45; Issekutz et al., 1991, J. Immunol., 147: 109 (TA-2 MAb)). These anti-VLA-4 monoclonal antibodies and other anti-VLA-4 antibodies (e.g., U.S. Pat. No. 5,888,507—Biogen, Inc. and references cited therein) capable of recognizing the alpha and/or beta chain of VLA-4 will be useful in the methods of treatment according to the present invention. AntiVLA-4 antibodies that will recognize the VLA-4 α₄ chain epitopes involved in binding to VCAM-1 and fibronectin ligands (i.e., antibodies which can bind to VLA-4 at a site involved in ligand recognition and block VCAM-1 and fibronectin binding) are preferred. Such antibodies have been defined as B epitope-specific antibodies (B1 or B2) (Pulido et al., 1991, supra) and are also anti-VLA-4 antibodies according to the present invention.

Fully human monoclonal antibody homologs against VLA-4 are another preferred binding agent that may block or coat VLA-4 ligands in the method of the invention. In their intact form these may be prepared using in vitro-primed human splenocytes, as described by Boerner et al., 1991, J. Immunol., 147: 86-95. Alternatively, they may be prepared by repertoire cloning as described by Persson et al., 1991, Proc. Nat. Acad. Sci. USA, 88: 2432-36 or by Huang et al., 1991, J. Immunol. Meth., 141: 227-236. U.S. Pat. No. 5,798,230 (Aug. 25, 1998, “Process for the preparation of human monoclonal antibodies and their use”) describes preparation of human monoclonal antibodies from human B cells. According to this process, human antibody-producing B cells are immortalized by infection with an Epstein-Barr virus, or a derivative thereof, that expresses Epstein-Barr virus nuclear antigen 2 (EBNA2). EBNA2 function, which is required for immortalization, is subsequently shut off, which results in an increase in antibody production. Additional methods are known in the art.

For yet another method for producing fully human antibodies, see, e.g., U.S. Pat. No. 5,789,650, which describes transgenic non-human animals capable of producing heterologous antibodies and transgenic non-human animals having inactivated endogenous immunoglobulin genes. Endogenous immunoglobulin genes are suppressed by antisense polynucleotides and/or by antiserum directed against endogenous immunoglobulins. Heterologous antibodies are encoded by immunoglobulin genes not normally found in the genome of that species of non-human animal. One or more transgenes containing sequences of unrearranged heterologous human immunoglobulin heavy chains are introduced into a non-human animal thereby forming a transgenic animal capable of functionally rearranging transgenic immunoglobulin sequences and producing a repertoire of antibodies of various isotypes encoded by human immunoglobulin genes. Such heterologous human antibodies are produced in B-cells, which are thereafter immortalized, e.g., by fusing with an immortalizing cell line such as a myeloma or by manipulating such B-cells by other techniques to perpetuate a cell line capable of producing a monoclonal, heterologous, fully human antibody homolog. Large non-immunized human phage display libraries may also be used to isolate high affinity antibodies that can be developed as human therapeutics using standard phage technology.

Following the early methods for the preparation of true “chimeric antibodies” (i.e., where the entire constant and entire variable regions are derived from different sources), a new approach was described in EP 0239400 (Winter et al.) whereby antibodies are altered by substitution (within a given variable region) of their complementarity determining regions (CDRs) for one species with those from another. This process may be used, for example, to substitute the CDRs from human heavy and light chain Ig variable region domains with alternative CDRs from murine variable region domains. These altered Ig variable regions may subsequently be combined with human Ig constant regions to created antibodies, which are totally human in composition except for the substituted murine CDRs. Such CDR-substituted antibodies would be predicted to be less likely to elicit an immune response in humans compared to true chimeric antibodies, because the CDR-substituted antibodies contain considerably less non-human components. The process for humanizing monoclonal antibodies via CDR “grafting” has been termed “reshaping” (Riechmann et al., 1988, Nature 332: 323-7; and Verhoeyen et al., 1988, Science 239: 1534-6).

6.5 Antibody Purification

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10: 163-7 (1992) describe a procedure for isolating antibodies, which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. In instances when the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells is preferably subjected to at least one purification step prior to LPHIC. Examples of suitable purification steps include hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., 1983 J. Immunol. Meth. 62: 1-13). Protein G is recommended for all mouse isotypes and for human γ3 (Guss et al., 1986 EMBO J. 5: 1567-75). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminant(s) is subjected to LPHIC. Often, the antibody composition to be purified will be present in a buffer from the previous purification step. However, it may be necessary to add a buffer to the antibody composition prior to the LPHIC step. Many buffers are available and can be selected by routine experimentation. The pH of the mixture comprising the antibody to be purified and at least one contaminant in a loading buffer is adjusted to a pH of about 2,5-4.5 using either an acid or base, depending on the starting pH. Preferably, the loading buffer has a low salt concentration (i.e., less than about 0.25 M salt).

The mixture is loaded on the HIC column. HIC columns normally comprise a base matrix (e.g., cross-linked agarose or synthetic copolymer material) to which hydrophobic ligands (e.g., alkyl or aryl groups) are coupled. A preferred HIC column comprises an agarose resin substituted with phenyl groups (e.g., a Phenyl SEPHAROSE™ column). Many HIC columns are available commercially. Examples include, but are not limited to, Phenyl SEPHAROSE 6 FAST FLOW™ column with low or high substitution (Pharmacia LKB Biotechnology, AB, Sweden); Phenyl SEPHAROSE™ High Performance column (Pharmacia LKB Biotechnology, AB, Sweden); Octyl SEPHAROSE™ High Performance column (Pharmacia LKB Biotechnology, AB, Sweden); FRACTOGEL™ EMD Propyl or FRACTOGEL™ EMD Phenyl columns (E. Merck, Germany); MACRO-PREPT™ Methyl or MACRO-PREP™ t-Butyl Supports (Bio-Rad, California); WP HI-Propyl (C₃)™ column (J. T. Baker, New Jersey); and TOYOPEARL™ ether, phenyl or butyl columns (TosoHaas, PA).

The antibody is eluted from the column using an elution buffer, which is normally the same as the loading buffer. The elution buffer can be selected using routine experimentation. The pH of the elution buffer is between about 2,5-4.5 and has a low salt concentration (i.e., less than about 0.25 M salt). It has been discovered that it is not necessary to use a salt gradient to elute the antibody of interest; the desired product is recovered in the flow through fraction, which does not bind significantly to the column.

The LPHIC step provides a way to remove a correctly folded and disulfide bonded antibody from unwanted contaminants (e.g., incorrectly associated light and heavy fragments). In particular, the method provides a means to substantially remove an impurity characterized herein as a correctly folded antibody fragment whose light and heavy chains fail to associate through disulfide bonding.

Diagnostic or therapeutic formulations of the purified protein can be made by providing the antibody composition in the form of a physiologically acceptable carrier, examples of which are provided below.

To remove contaminants (e.g., unfolded antibody and incorrectly associated light and heavy fragments) from the HIC column so that it can be re-used, a composition including urea (e.g., 6.0 M urea, 1% MES buffer pH 6.0, 4 mM ammonium sulfate) can be flowed through the column. Other methods are known in the art.

6.6 Immunoglobulin Formulations

Antibodies and immunoglobulins having the desired therapeutic effect may be administered in a physiologically acceptable carrier to a subject. The antibodies may be administered in a variety of ways including but not limited to parenteral administration, including subcutaneous, subdural, intravenous, intramuscular, intrathecal, intraperitoneal, intracerebral, intraarterial, or intralesional routes of administration, localized (e.g., surgical application or surgical suppository), and pulmonary (e.g., aerosols, inhalation, or powder) and as described further below.

Depending upon the manner of introduction, the immunoglobulins may be formulated in a variety of ways. The concentration of therapeutically active immunoglobulin in the formulation (i.e., a formulation sufficient to diagnose and/or treat imflammatory bowel conditions) may vary from about 1 mg/ml to 1 g/ml. Preferably, the immunoglobulin composition, when administered to a subject in need thereof, reaches a blood level of immunoglobulin in the subject of about 10 ng/ml or more.

Preferably, the immunoglobulin is formulated for parenteral administration in a suitable inert carrier, such as a sterile physiological saline solution. For example, the concentration of immunoglobulin in the carrier solution is typically between about 1-100 mg/ml. The dose administered will be determined by route of administration. Preferred routes of administration include parenteral or intravenous administration.

For parenteral administration, the antibodies of the invention can be administered as injectionable dosages of a solution or suspension of the substance in a physiologically acceptable diluent with a pharmaceutical carrier which can be a sterile liquid such as water and oils with or without the addition of a surfactant and other pharmaceutically preparations are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. The antibodies of this invention can be administered in the form of a depot injection or implant preparation, which can be formulated in such a manner as to permit a sustained release of the active ingredient. A preferred composition comprises monoclonal antibody at 5 mg/mL, formulated in aqueous buffer consisting of 50 mM L-histidine, 150 mM NaCl, adjusted to pH 6.0 with HCl.

According to an important feature of the invention, an immunoglobulin that recognizes and binds to VLA-4 may be administered alone, or in combination with an another agent which is typically used to treat inflammatory bowel disease such as Crohns's disease, asthma, multiple sclerosis (MS), rheumatoid arthritis (RA), graft versus host disease (GVHD), host versus graft disease, and various spondyloarthropathies. Administration of of these agents can occur prior to, concurrent with or after administration with the immunoglobulin. Preferably, the other agent is not a steroid.

A therapeutically effective amount of an antibody or immunoglobulin, e.g., natalizumab, can be estimated by comparison with established effective doses for known antibodies, taken together with data obtained for natalizumab in both in vivo and in vitro models. Preferably the data is from studies of treatment of inflammatory bowel disease such as Crohns's disease, asthma, multiple sclerosis (MS), rheumatoid arthritis (RA), graft versus host disease (GVHD), host versus graft disease, and spondyloarthropathies, as appropriate. As is known in the art, adjustments in the dose may be necessary due to immunoglobulin degeneration or metabolism, systemic versus localized delivery, as well as the age, body weight, general health, sex, diet, time of administration, drug interactions and the severity of the condition of the subject to whom the immunoglobulin is administered. Such adjustments may be made and appropriate doses determined by one of skill in the art through routine experimentation.

Therapeutic formulations of the immunoglobulin are prepared for storage by mixing the immunoglobulin having the desired degree of purity with optional physiologically acceptable carriers, excipients, or stabilizers (REMINGTON'S PHARMACEUTICAL SCIENCES, 16^{th} ed., A. Osol, Ed., 1980 and more recent editions), in the form of lyophilized cake or aqueous solutions. Acceptable immunoglobulin carriers, excipients or stabilizers are nontoxic, nontherapeutic and/or non-immunogenic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG). Specific examples of carrier molecules include but are not limited to glycosaminoglycans (e.g., heparin sulfate), hyaluronic acid, keratan-sulfate, chondroitin 4-sulfate, chondroitin 6-sulfate, heparan sulfate and dermatin sulfate, perlecan and pentopolysulfate.

Pharmaceutical compositions comprising immunoglobulins can also include if desired, pharmaceutically acceptable, non-toxic carriers or diluents, which are vehicles commonly used to formulated pharmaceutical compositions for animal or human administration. The diluent is selected so as not to affect the biological activity of the combination. Examples include but are not limited to distilled water, physiological phosphate-buffered saline, Ringer's solutions, dextrose solution, and Hank's solution.

The agents of the invention can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol. The formulations may also contain conventional additives, such as solubilizers, isotonic agents, suspending agents, emulsifying agents, stabilizers and preservatives.

The immunoglobulins may also be utilized in aerosol formulation to be administered via inhalation or pulmonary delivery. The agents of the present invention can be formulated into pressurized acceptable propellants such as dichlorodifluoromethane, propane, nitrogen and the like.

The immunoglobulin also may be entrapped in microcapsules prepared, for example, by coacervation techniques or by interfacial polymerization (e.g., hydroxymethylcellulose or gelatin-microcapsules and poly-methylmethacylate microcapsules), in colloidal drug delivery systems (e.g., liposomes, albumin microspheres, microemulsions, nano-particles and nanocapsules), or in macroemulsions. Such techniques are disclosed in REMINGTON'S PHARMACEUTICAL SCIENCES, supra.

The immunoglobulin to be used for in vivo administration must be sterile. This is readily accomplished by filtration through sterile filtration membranes, prior to or following lyophilization and reconstitution. The immunoglobulin ordinarily will be stored in lyophilized form or in solution.

Therapeutic immunoglobulin compositions generally are placed into a container having a sterile access port, for example, an intravenous solution bag or vial having a stopper pierceable by a hypodermic injection needle or similar sharp instrument.

Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15: 167-277 (1981) and Langer, Chem. Tech. 12: 98-105 (1982) or poly(vinylalcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for immunoglobulin stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, developing specific polymer matrix compositions, and the like.

Sustained-release immunoglobulin compositions also include liposomally entrapped immunoglobulin. Liposomes containing the immunoglobulin are prepared by methods known per se. See, e.g., Epstein et al., Proc. Natl. Acad. Sci. USA 82: 3688-92 (1985); Hwang et al., Proc. Natl. Acad Sci. USA 77: 4030-4 (1980); U.S. Pat. Nos. 4,485,045; 4,544,545; 6,139,869; and 6,027,726. Ordinarily, the liposomes are of the small (about 200 to about 800 Angstroms), unilamellar type in which the lipid content is greater than about 30 mole percent (mol. %) cholesterol; the selected proportion being adjusted for the optimal immunoglobulin therapy.

The immunoglobulins of this invention can be administered in a sustained release form, for example a depot injection, implant preparation, or osmotic pump, which can be formulated in such a manner as to permit a sustained release of the active ingredient. Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of protein deposits (e.g., the site of formation of amyloid deposits associated with neurodegenerative disorders), so that the local concentration of active agent is increased at that site relative to the rest of the body.

In addition, immunoglobulins which diagnose and/or treat imflammatory bowel conditions may be provided by administering a polynucleotide encoding a whole or partial antibody (e.g., a single chain Fv) to a subject. The polynucleotide is administered to a subject in an appropriate vehicle to allow the expression of the immunoglobulin in the subject in a therapeutically effective amount.

A typical daily dosage might range for immunoglobulins ranges from about 1 μg/kg to up to about 10 mg/kg or more, depending on the factors mentioned herein. Typically, the clinician will administer immunoglobulin until a dosage is reached that achieves the desired effect. The progress of this therapy is easily monitored by conventional assays.

A “stable” antibody or antibody fragment formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, (Vincent Lee Ed., Marcel Dekker, Inc., New York, N.Y., Pubs. 1991) and A. Jones, Adv. Drug Delivery Rev. 10: 29-90 (1993), for example. Stability can be measured at a selected temperature for a selected time period. Preferably, the formulation is stable at room temperature (about 30° C.) or at 40° C. for at least 1 month and/or stable at about 2-8° C. for at least 1 year for at least about 2 years. Furthermore, the formulation is preferably stable following freezing (to, e.g., −70° C.) and thawing of the formulation.

A protein “retains its physical stability” in a pharmaceutical formulation if it shows no signs of aggregation, precipitation and/or denaturation upon visual examination of color and/or clarity, or as measured by IV light scattering or by size exclusion chromatography.

A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g., clipping) that can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation) that can be evaluated by, e.g., ion-exchange chromatography.

An immunoglobulin “retains its biological activity” in a pharmaceutical formulation, if the biological activity of the immunoglobulin at a given time is within about 10% (within the errors of the assay) of the biological activity exhibited at the time the pharmaceutical formulation was prepared as determined in an antigen-binding assay, for example.

6.7 Routes of Administration of Immunoglobulin Compositions

The pharmaceutical compositions discussed supra can be administered for diagnosis, prophylactic and/or therapeutic treatments of inflammatory bowel diseases, asthma, multiple sclerosis (MS), rheumatoid arthritis (RA), graft versus host disease (GVHD), host versus graft disease, and various spondyloarthropathies. In therapeutic applications, compositions are administered to a patient suspected of, or already suffering from a disease, in an amount sufficient to provide treatment. An amount adequate to accomplish this is defined as a therapeutically or pharmaceutically effective dose.

The pharmaceutical compositions will be administered by parenteral, topical, intravenous, oral, or subcutaneous, intramuscular local administration, such as by aerosol or transdermally, for prophylactic and/or therapeutic treatment. Although the proteinaceous substances of this invention may survive passage through the gut following oral administration (p.o.), subcutaneous (s.c.), intravenous (i.v.), intramuscular (i.m.), intraperitoneal administration by depot injection; or by implant preparation are preferred.

The pharmaceutical compositions can be administered in a variety of unit dosage forms depending upon the method of administration. For example, unit dosage forms suitable for oral administration include powder, tablets, pills, capsules, and lozenges.

Effective doses of the compositions of the present invention, for the treatment of the above described conditions will vary depending upon many different factors, including means of administration, target site, physiological state of the patient, and other medicaments administered. Thus, treatment dosages will need to be titrated to optimize safety and efficacy. These compositions may be administered to mammals for veterinary use and for clinical use in humans in a manner similar to other therapeutic agents, ie., in a physiologically acceptable carrier. In general, the administration dosage will range from about 0.0001 to 100 mg/kg, and more usually 0.01 to 0.5 mg/kg of the host body weight.

In a preferred treatment regime, the antibody is administered by intravenous infusion or subcutaneous injection at a dose from 1 to 5 mg antibody per kilo of patient bodyweight. The dose is repeated at interval from 2 to 8 weeks. Within this range, the preferred treatment regimen is 3 mg antibody per kilo of bodyweight repeated at a 4-week interval.

7. Drug Combinations

Other drugs are currently used for the treatment of inflammatory bowel diseases, asthma, multiple sclerosis (MS), rheumatoid arthritis (RA), graft versus host disease (GVHD), host versus graft disease, and various spondyloarthropathies. These and other drugs are also contemplated for use in combination with the compounds and compositions disclosed herein. Selection of one or more agent to be utilized in a cocktail and/or combination with the compounds and compositions disclosed herein will be dependent on the management of the disease. For example, the compounds and compositions disclosed herein can be administered with immunosuppressant agents to further treat Crohns's disease and to suppress symptoms.

Dosage forms of the agents to be used in combination with the compounds and compositions disclosed herein would vary depending on the subject and drug combination being utilized.

8. Chronic Administration Dosage Regimens

The chronic treatment regimen of the present invention provides an α₄ integrin agent (e.g., small molecule or immunoglobulin) at a level that will maintain sufficient receptor saturation to suppress pathological inflammation in a patient in need of such. The methods of the invention entails administration once per every two weeks or once a month to once every two months, with repeated dosings taking place over a period of at least six months, and more preferably for a year or longer. The methods of the invention involve obtaining and maintaining a receptor saturation level in a human patient of a dimer comprising α₄ integrin (e.g., VLA-4) in a range of from about 65% to 100%, more preferably between 75%, to 100%, and even more preferably between 80-100%. These receptor saturation levels are maintained at these levels chronically (e.g., over a period of 6 months or so) to allow for continued suppression of pathological inflammation.

In a specific embodiment, the treatment agent is an antibody, preferably a humanized or human antibody, and the dosing is on a monthly basis. Levels of receptor saturation can be monitored to determine the efficacy of the dosing regime, and physiological markers measured to confirm the success of the dosage regime. As a confirmation, serum levels of the antibody can be monitored to identify clearance of the antibody and to determine the potential effect of half-life on the efficacy of the treatment.

In another specific embodiment, the treatment agent is a small molecule compound, and the dosing is on a monthly basis. Levels of saturation may be monitored to determine the efficacy of the dosing regime, and physiological markers measured to confirm the success of the dosage regime.

For treatment with an agent of the invention, the dosage ranges from about 0.0001 to 100 mg/kg, and more usually 0.01 to 5 mg/kg, of the host body weight. For example dosages can be 1 mg/kg body weight or 10 mg/kg body weight. Dosage and frequency vary depending on the half-life of the agent in the patient. The dosage and frequency of administration can vary depending on whether the treatment is prophylactic or therapeutic. For immunoglobulin administration, each dosing injection is generally between 2.0 to 8.0 mg/kg dosage. For a compound administration, each dosing injection is generally between 1.0 to 50.0 mg/kg dosage. In accordance with the teachings provided herein, effective dosages can be monitored by obtaining a fluid sample from a patient. For this, generally a blood serum or cerebrospinal fluid sample is taken and integrin receptor saturation is determined using methods well known in the art. Ideally, a sample is taken prior to initial dosing; subsequent samples are taken and measured prior to and/or after each treatment.

As an alternative to chronic administration comprised of repeated individual dosings, a agent for the treatment of inflammatory bowel diseases, asthma, multiple sclerosis (MS), rheumatoid arthritis (RA), graft versus host disease (GVHD), host versus graft disease, and various spondyloarthropathies can be administered as a sustained release formulation, provided the dosage is such that the levels of receptor saturation remain sufficient to suppress inflammation. For example, controlled release systems can be used to chronically administer an agent within the scope of this invention. Discussions of appropriate controlled release dosage forms may be found in Lesczek Krowczynski, EXTENDED-RELEASE DOSAGE FORMS, 1987 (CRC Press, Inc.).

The various controlled release technologies cover a very broad spectrum of drug dosage forms. Controlled release technologies include, but are not limited to physical systems and chemical systems. Physical systems include, but not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., non-erodible, erodible, environmental agent ingression, and degradable), and materials physically dispersed in non-porous, polymeric, or elastomeric matrices (e.g., non-erodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, CONTROLLED RELEASE TECHNOLOGIES: METHODS, THEORY AND APPLICATIONS, 1980 (CRC Press, Inc.).

The methods of the invention can be used to treat a patient that is affected with a disorder involving or arising from pathological inflammation, or to prophylactically treat a patient at risk for a particular disorder. The dosage regimens necessary for prophylactic versus therapeutic treatment can vary, and will need to be designed for the specific use and disorder treated.

In some methods, two or more agents (e.g., monoclonal antibodies with different binding specificities, a monoclonal antibody and a compound as disclosed herein) are administered concurrently, in which case the dosage of each agent administered falls within the ranges indicated. Combination therapies can also occur where the agents are administered consecutively to the patient with a desired time interval been periods of administration. Intervals can also be irregular as indicated by measuring receptor saturation levels or by following other indicia of the disease process.

Those of skill will readily appreciate that dose levels can vary as a function of the specific agent, the severity of the symptoms and the susceptibility of the subject to side effects. Some of the specific agents are more potent than others. Preferred dosages for a given agent are readily determinable by those of skill in the art by a variety of means. A preferred means is to measure the physiological potency of a given agent.

In prophylactic applications, pharmaceutical compositions are chronically administered to a patient susceptible to, or otherwise at risk of, a particular disease in an amount sufficient to eliminate or reduce the risk or delay the outset of the disease. Such an amount is defined to be a prophylactically effective dose. In patients with multiple sclerosis in remission, risk may be assessed by NMR imaging or, in some cases, by pre-symptomatic indications observed by the patient.

Effective dosage regimes of the compositions of the present invention, for the treatment of the above described conditions will vary depending upon many different factors, including means of administration, target site, physiological state of the patient, and other medicaments administered. Thus, treatment dosages will need to be titrated to optimize safety and efficacy. In general, each administration of the dosage regimen will range from about 0.0001 to about 100 mg/kg, usually about 0.01 to about 50, and more usually from about 0.1 to about 30 mg/kg of the host body weight.

9. Testing Reagents

Reagents can be tested in vitro and in vivo. Many in vitro models exist to test whether a reagent binds to the α₄ subunit, as would be known in the art. Testing whether the reagent has activity in vivo at diagnosis and/or treatment of imflammatory bowel conditions, as well as other inflammatory conditions, can be performed using the experimental autoimmune encephalomyelitis (EAE) animal model. EAE is an inflammatory condition of the central nervous system with similarities to multiple sclerosis (Paterson, IN TEXTBOOK OF IMMUNOPATHOLOGY, eds. Miescher and Mueller-Eberhard, 179-213, Grune and Stratton, N.Y. 1976).

Sections of EAE brain can be tested for their ability to support leukocyte attachment using, for example, an in vitro binding assay described in Stamper and Woodruff, J. Exp. Med. 144: 828-833 (1976). Reagents against leukocyte adhesion receptors can be examined for inhibitory activity in the in vitro section assay. The attachment of U937 cells (a human monocytic cell line) was almost completely blocked by antibodies against human VLA-4 integrin. The antibodies produced significantly greater blocking effect as compared to antibodies against other adhesion molecules.

Surprisingly, antibodies that selectively inhibit the fibronectin binding activity of α₄ integrin (P4G9 and HP1/7) enhanced U937 attachment to the EAE vessels. These results suggest that fibronectin-binding activity of α₄ integrin is not directly involved in U937 adhesion to EAE vessels in vitro. Given the in vitro results using the α₄β₁ reagents described above, the effect of these antibodies on the progression of EAE can also be tested in vivo by measuring the delay in the onset of paralysis or reduction in severity of the paralysis.

Additional reagents effective for diagnosis and/or treatment of imflammatory bowel conditions can be identified by use of adhesion assays. Using HP2/1 or N-[N-(3-pyridinesulfonyl)-L-3,3-dimethyl-4-thiaprolyl]-O-[1-methylpiperazin-4-ylcarbonyl]-L-tyrosine isopropyl ester as a control for example, other antibodies or reagents can be screened for their ability to inhibit the binding of lymphocytes to a known ligand for α₄β₁ integrin. Several additional reagents can be identified that inhibit adhesion by targeting the α₄ subunit of the VLA-4 leukocyte cell surface receptor.

Monoclonal antibodies useful in the methods and compositions of the present invention include for example HP2/1, TY21.6, TY21.12, and L25 as discussed in U.S. Pat. No. 6,033,665, which is herein incorporated by reference in its entirety. These antibodies react with the α chain of VLA-4 and block binding to VCAM-1, fibronectin and inflamed brain endothelial cells, but do not affect the activity of the other members of the β₁ integrin family.

Other reagents which selectively react against the VLA-4/VCAM-1 target can also be used. This reagent further would not affect matrix interactions (mediated by all members of the β₁ integrins) nor would it affect normal intestinal immunity (mediated by α₄P₇). The production of this and other such reagents are well within the skill of the art.

Assays for determining whether agents exhibit α4β1 and/or α4β7 activity are known to those of skill in the art.

For example, in an assay, the compounds can be bound to a solid support and the α4β7 integrin sample added thereto. The amount of α4β1 or α4β7 integrin in the sample can be determined by conventional methods such as use of a sandwich ELISA assay. In addition, certain of the compounds of this invention inhibit, in vivo, adhesion of leukocytes to endothelial cells and epithelial cells in mucosal organs mediated by α4β1 or α4β7 integrin and, accordingly, can be used in the treatment of diseases mediated by α4β1 or α4β7 integrin.

The biological activity of the compounds identified above may be assayed in a variety of systems. For example, a compound can be immobilized on a solid surface and adhesion of cells expressing α4β1 or α4β7 integrin can be measured. Using such formats, large numbers of compounds can be screened. Cells suitable for this assay include any leukocytes known to express α4β1 or α4β7 integrin such as memory T cells and eosinophils. A number of leukocyte cell lines can also be used, examples include the cell line RPMI-8866.

The compounds may also be tested for the ability to competitively inhibit binding between α4β1 or α4β7 integrin and MAdCAM-1, or between α4β1 or α4β7 integrin and a labeled compound known to bind α4β1 or α4β7 integrin such as a compound of this invention or antibodies to α4β7 integrin. In these assays, the MAdCAM-1 can be immobilized on a solid surface. MAdCAM-1 may also be expressed as a recombinant fusion protein having an Ig tail (e.g., IgG Fc) so that binding to α4β7 integrin may be detected in an immunoassay. Alternatively, MAdCAM-1 expressing cells, such as activated endothelial cells or MAdCAM-1 transfected fibroblasts can be used.

Both α4β7 and α4β1 can mediate adhesion to VCAM-1 and to fibronectin. For assays which measure the ability to block adhesion to VCAM-1 and to fibronectin, the assays described in International Patent Application Publication No. WO US98/15324 are particularly preferred. This application is incorporated herein by reference in its entirety.

Many assay formats employ labelled assay components. The labelling systems can be in a variety of forms. The label may be coupled directly or indirectly to the desired component of the assay according to methods well known in the art. A wide variety of labels may be used. The component may be labelled by any one of several methods. The most common method of detection is the use of autoradiography with 3H, 125I, 35S, 14C, or 32P labelled compounds or the like. Non-radioactive labels include ligands which bind to labelled antibodies, fluorophores, chemiluminescent agents, enzymes and antibodies which can serve as specific binding pair members for a labelled ligand. The choice of label depends on sensitivity required, ease of conjugation with the compound, stability requirements, and available instrumentation.

Appropriate in vivo models for demonstrating efficacy in treating inflammatory responses include EAE (experimental autoimmune encephalomyelitis) in mice, rats, guinea pigs or primates, as well as other inflammatory models dependent upon α₄ integrins.

Compounds having the desired biological activity may be modified as necessary to provide desired properties such as improved pharmacological properties (e.g., in vivo stability, bio-availability), or the ability to be detected in diagnostic applications. For instance, inclusion of one or more D-amino acids in the sulfonamides of this invention typically increases in vivo stability. Stability can be assayed in a variety of ways such as by measuring the half-life of the proteins during incubation with peptidases or human plasma or serum. A number of such protein stability assays have been described (see, e.g., Verhoef et al., Eur. J. Drug Metab. Pharmacokinet., 1990, 15(2):83-93).

10. Synthesis of Compounds

The following examples are put forth so as to provide those of ordinary skill in the art with a complete disclosure and description of how to make and use the present invention, and is not intended to limit the scope of what the inventors regard as their invention nor is it intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

10.1 Synthesis of Compounds of Formulae I and II

In the examples below, if an abbreviation is not defined above, it has its generally accepted meaning. Further, all temperatures are in degrees Celsius (unless otherwise indicated). The following Methods were used to prepare the compounds set forth below as indicated.

### Method 1

### N-Tosylation Procedure

N-Tosylation of the appropriate amino acid was conducted via the method of Cupps, Boutin and Rapoport J. Org. Chem. 1985, 50, 3972.

### Method 2

### Methyl Ester Preparation Procedure

Amino acid methyl esters were prepared using the method of Brenner and Huber Helv. Chim. Acta 1953, 36, 1109.

### Method 3

### BOP Coupling Procedure

The desired dipeptide ester was prepared by the reaction of a suitable N-protected amino acid (1 equivalent) with the appropriate amino acid ester or amino acid ester hydrochloride (1 equivalent), benzotriazol-1-yloxy-tris(dimethylamino)phosphonium hexafluorophosphate [BOP] (2.0 equivalent), triethylamine (1.1 equivalent), and DMF. The reaction mixture was stirred at room temperature overnight. The crude product is purified flash chromatography to afford the dipeptide ester.

### Method 4

### Hydrogenation Procedure I

Hydrogenation was performed using 10% palladium on carbon (10% by weight) in methanol at 30 psi overnight. The mixture was filtered through a pad of Celite and the filtrate concentrated to yield the desired amino compound.

### Method 5

### Hydrolysis Procedure I

To a chilled (0° C.) THF/H₂O solution (2:1, 5-10 mL) of the appropriate ester was added LiOH (or NaOH) (0.95 equivalents). The temperature was maintained at 0° C. and the reaction was complete in 1-3 hours. The reaction mixture was extracted with ethyl acetate and the aqueous phase was lyophilized resulting in the desired carboxylate salt.

### Method 6

### Ester Hydrolysis Procedure II

To a chilled (0° C.) THF/H₂O solution (2:1, 5-10 mL) of the appropriate ester was added LiOH (1.1 equivalents). The temperature was maintained at 0° C. and the reaction was complete in 1-3 hours. The reaction mixture was concentrated and the residue was taken up into H₂O and the pH adjusted to 2-3 with aqueous HCl. The product was extracted with ethyl acetate and the combined organic phase was washed with brine, dried over MgSO₄, filtered and concentrated to yield the desired acid.

### Method 7

### Ester Hydrolysis Procedure III

The appropriate ester was dissolved in dioxane/H₂O (1:1) and 0.9 equivalents of 0.5 N NaOH was added. The reaction was stirred for 3-16 hours and than concentrated. The resulting residue was dissolved in H₂O and extracted with ethyl acetate. The aqueous phase was lyophilized to yield the desired carboxylate sodium salt.

### Method 8

### Sulfonylation Procedure I

To the appropriately protected aminophenylalanine analog (11.2 mmol), dissolved in methylene chloride (25 ml) and cooled to −78° C. was added the desired sulfonyl chloride (12 mmol) followed by dropwise addition of pyridine (2 mL). The solution was allowed to warm to room temperature and was stirred for 48 hr. The reaction solution was transferred to a 250 mL separatory funnel with methylene chloride (100 mL) and extracted with 1N HCl (50 mL×3), brine (50 mL), and water (100 mL). The organic phase was dried (MgSO₄) and the solvent concentrated to yield the desired product.

### Method 9

### Reductive Amination Procedure

Reductive amination of Tos-Pro-p-NH₂-Phe with the appropriate aldehyde was conducted using acetic acid, sodium triacetoxyborohydride, methylene chloride and the combined mixture was stirred at room temperature overnight. The crude product was purified by flash chromatography.

### Method 10

### BOC Removal Procedure

Anhydrous hydrochloride (HCl) gas was bubbled through a methanolic solution of the appropriate Boc-amino acid ester at 0° C. for 15 minutes and the reaction mixture was stirred for three hours. The solution was concentrated to a syrup and dissolved in Et₂O and reconcentrated. This procedure was repeated and the resulting solid was placed under high vacuum overnight.

### Method 11

### tert-Butyl Ester Hydrolysis Procedure I

The tert-butyl ester was dissolved in CH₂Cl₂ and treated with TFA. The reaction was complete in 1-3 hr at which time the reaction mixture was concentrated and the residue dissolved in H₂O and lyophilized to yield the desired acid.

### Method 12

### EDC Coupling Procedure I

To a CH₂Cl₂ solution (5-20 mL) of N-(toluene-4-sulfonyl)-L-proline (1 equivalent), the appropriate amino acid ester hydrochloride (1 equivalent), N-methylmorpholine (1,1-2.2 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight. The reaction mixture was poured into H₂O and the organic phase was washed with sat. NaHCO₃, brine, dried (MgSO₄ or Na₂SO₄), filtered and concentrated. The crude product was purified by column chromatography.

### Method 13

### EDC Coupling Procedure II

To a DMF solution (5-20 mL) of the appropriate N-protected amino acid (1 equivalent), the appropriated amino acid ester hydrochloride (1 equivalent), Et₃N (1.1 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight. The reaction mixture was partitioned between EtOAc and H₂O and the organic phase washed with 0.2 N citric acid, H₂O, sat. NaHCO₃, brine, dried (MgSO₄ or Na₂SO₄), filtered and concentrated. The crude product was purified by column chromatography or preparative TLC.

### Method 14

### Sulfonylation Procedure II

The appropriate sulfonyl chloride was dissolved in CH₂Cl₂ and placed in an ice bath. L-Pro-L-Phe-OMe.HCl (1 equivalent) and Et₃N (1.1 equivalent) was added and the reaction allowed to warm to room temperature and stirred overnight under an atmosphere of nitrogen. The reaction mixture was concentrated and the residue partitioned between EtOAc and H₂O and the organic phase washed with sat. NaHCO₃, brine, dried (MgSO₄ or Na₂SO₄), filtered and concentrated. The crude product was purified by column chromatography or preparative TLC.

### Method 15

### Sulfonylation Procedure III

To a solution of L-Pro-L-4-(3-dimethylaminopropyloxy)-Phe-OMe [prepared using the procedure described in Method 10] (1 equivalent) in CH₂Cl₂ was added Et₃N (5 equivalents) followed by the appropriate sulfonyl chloride (1.1 equivalent). The reaction was allowed to warm to room temperature and stirred overnite under an atmosphere of nitrogen. The mixture was concentrated, dissolved in EtOAc, washed with sat. NaHCO₃ and 0.2 N citric acid. The aqueous phase was made basic with solid NaHCO₃ and the product extracted with EtOAc. The organic phase was washed with brine, dried (MgSO₄ or Na₂SO₄), filtered and concentrated. The crude methyl ester was purified by preparative TLC. The corresponding acid was prepared using the procedure described in Method 7.

### Method 16

### Hydrogenation Procedure II

To a methanol (10-15 mL) solution of the azlactone was added NaOAc (1 equivalent) and 10% Pd/C. This mixture was placed on the hydrogenator at 40 psi H₂. After 8-16 hours, the reaction mixture was filtered through a pad of Celite and the filtrate concentrated to yield the dehydrodipeptide methyl ester. The ester was dissolved in dioxane/H₂O (5-10 mL), to which was added 0.5 N NaOH (1.05 equivalents). After stirring for 1-3 hours, the reaction mix was concentrated and the residue was redissolved in H₂O and washed with EtOAc. The aqueous phase was made acidic with 0.2 N HCl and the product was extracted with EtOAc. The combined organic phase was washed with brine (1×5 mL), dried (MgSO₄ or Na₂SO₄), filtered and concentrated to yield the acid as approximately a 1:1 mixture of diastereomers.

### Method 17

### tert-Butyl Ester Hydrolysis Procedure II

The tert-butyl ester was dissolved in CH₂Cl₂ (5 mL) and treated with TFA (5 mL). The reaction was complete in 1-3 hours at which time the reaction mixture was concentrated and the residue dissolved in H₂O and concentrated. The residue was redissolved in H₂O and lyophilized to yield the desired product.

### EXAMPLE 1

### Synthesis of N-(Toluene-4-sulfonyl)-L-prolyl-L-4-(4-methylpiperazin-1-ylcarbonyloxy)phenylalanine Ethyl Ester

The title compound was prepared following the procedure outlined for the preparation of Example 4 and substitution of appropriate starting materials.

NMR data was as follows:

¹H NMR (CD₃)₂SO): δ=8.33 (d, 1H), 7.70 (d, 2H), 7.41 (d, 2H), 7.24 (d, 2H), 7.00 (d, 2H), 4.52-4.44 (m, 1H), 4.09-4.00 (m, 3H), 3.53 (bs, 2H), 3.38-3.31 (m, 3H), 3.11-3.01 (m, 3H), 2.39 (s, 3H), 2.32 (bs, 4H), 2.19 (s, 3H), 1.61-1.50 (m, 3H), 1.43-1.38 (m, 1H), 1.13 (t, 3H).

¹³C NMR (CD₃)₂SO): δ=171.1, 171.1, 153.9, 149.8, 143.6, 134.1, 133.9, 130.0, 129.8, 127.4, 121.5, 61.2, 60.7, 54.2, 54.1, 53.3, 49.0, 45.7, 44.0, 43.4, 35.8, 30.5, 23.8, 21.0, 14.0.

### EXAMPLE 2

### Synthesis of N-(Toluene-4-sulfonyl)-L-prolyl-L-4-(N,N-dimethylcarbamyloxy)phenylalanine Ethyl Ester

Into a reaction vial were combined 7.00 g (15.2 mmol, 1.0 eq) Ts-Pro-Tyr(H)-OEt and 1.86 g (15.2 mmol, 1.0 eq) DMAP. Methylene chloride (50 mL), triethylamine (2.12 mL—1.54 g, 15.2 mmol, 1.0 eq), and dimethylcarbamyl chloride (1.68 mL—1.96 g, 18.2 mmol, 1.2 eq) were then added. The vial was capped tightly, and the reaction solution swirled to obtain a homogeneous solution. The reaction solution was then heated to 40° C. After 48 h, TLC of the resulting colorless solution indicated complete conversion. The workup of the reaction solution was as follows: add 50 mL EtOAc and 50 mL hexanes to the reaction mixture, and wash with 3×50 mL 0.5 mL hexanes to the reaction mixture, and wash with 3×50 mL 0.5 M citric acid, 2×50 mL water, 2×50 mL 10% K₂CO₃, and 1×50 mL sat. NaCl. Dry with MgSO₄. Filter. Evaporate to obtain 8.00 g (99%) of the title compound as a clear oil, which solidifies upon standing. Recrystallize from 5:3:2 heptane/EtOAc/CH₂Cl₂.

NMR data was as follows:

¹H NMR (CD₃)₂SO): δ=8.32 (d, 1H), 7.70 (d, 2H), 7.41 (d, 2H), 7.23 (d, 2H), 7.00 (d, 2H), 4.52-4.44 (m, 1H), 4.09-4.02 (m, 3H), 3.37-3.31 (m, 1H), 3.11-2.96 (m, 3H), 3.00 (s, 3H), 2.87 (s, 3H), 2.39 (s, 3H), 1.61-1.50 (m, 3H), 1.43-1.38 (m, 1H), 1.13 (t, 3H).

¹³C NMR (CD₃)₂SO): δ=171.1, 171.1, 154.0, 150.0, 143.6, 133.9, 133.9, 130.0, 129.8, 127.4, 121.5, 61.2, 60.6, 53.3, 49.0, 36.3, 36.1, 35.8, 30.5, 23.8, 21.0, 14.0.

### EXAMPLE 3

### Synthesis of N-(Toluene-4-sulfonyl)-L-prolyl-L-4-(4-methylpiperazin-1-ylcarbonyloxy)phenylalanine Isopropyl Ester

The title compound was prepared following the procedure outlined for the preparation of Example 4 and substitution of appropriate starting materials.

NMR data was as follows:

¹H NMR (CDCl₃): δ =7.72 (d, 2H), 7.36 (d, 1H), 7.33 (d, 2H), 7.16 (d, 2H), 7.03 (d, 2H), 5.07 (Sept., 1H), 4.78 (dt, 1H), 4.08-4.05 (m, 1H), 3.67 (bs, 2H), 3.57 (bs, 2H), 3.41-3.35 (m, 1H), 3.24 (dd, 1H), 3.15-3.07 (m, 1H), 3.04 (dd, 1H), 3.46-2.43 (m, 7H), 2.34 (s, 3H), 2.05-2.02 (m, 1H).

¹³C NMR (CDCl₃): δ =170.9, 170.4, 153.6, 150.5, 144.3, 133.2, 133.1, 130.2, 130.0, 127.9, 121.7, 69.5, 62.2, 54.7, 53.4, 49.6, 46.1, 44.3, 43.7, 37.2, 29.7, 24.1, 21.6, 21.6, 21.4.

### EXAMPLE 4

### Synthesis of N-(Toluene-4-sulfonyl)-L-prolyl-L-4-(4-methylpiperazin-1-ylcarbonyloxy)phenylalanine tert-Butyl Ester

Combine 41.2 g (84.34 mmol, 1.0 eq) Ts-Pro-Tyr(H)-OtBu and 17.0 g (84.34 mmol, 1.0 eq) 4-nitrophenyl chloroformate. Add 700 mL CH₂Cl₂. Cap with a septum. Attach a N₂ line. Immerse the flask in a 4:1 water/EtOH+dry ice slurry, and stir to cool to −15° C. Add 29.38 mL (21.33 g, 210.81 mmol, 2.5 eq) Et₃N over five minutes with stirring. Stir at −10 to −15° C. for 1 h. Add 9.35 mL (8.45 g, 84.34 mmol, 1.0 eq) N-methyl piperazine over 3 minutes with stirring. Stir overnight while warming to room temperature. Dilute with 700 mL hexanes. Wash repeatedly with 10% K₂CO₃, until no yellow color (4-nitrophenol) is seen in the aqueous layer. Wash with sat. NaCl. Dry over anhydrous MgSO₄. Filter. Evaporate. Dissolve in 500 mL EtOH, and evaporate, to remove Et₃N. Repeat once. Dissolve in 400 mL EtOH, and add 600 mL water with stirring, to precipitate a solid or oil. If an oil, stir vigorously to solidify. Isolate the solid by filtration. Repeat dissolution, precipitation, and filtration, once. Rinse with water to remove traces of yellow color. High vacuum to constant mass yields the title compound as a white solid.

NMR data was as follows:

¹H NMR (CDCl₃): δ =7.72 (d, 2H), 7.33 (d, 3H), 7.17 (d, 2H), 7.02 (d, 2H), 4.71 (q, 1H), 4.09-4.06 (m, 1H), 3.67 (bs, 2H), 3.57 (bs, 2H), 3.41-3.34 (m, 1H), 3.22 (dd, 1H), 3.16-3.09 (m, 1H), 3.03 (dd, 1H), 2.46-2.43 (m, 7H), 2.34 (s, 3H), 2.05-2.02 (m, 1H), 1.57-1.43 (m, 3H), 1.47 (s, 9H).

¹³C NMR (CDCl₃): δ =171.8, 169.9, 153.6, 150.4, 144.3, 133.4, 133.1, 130.3, 130.0, 127.9, 121.6, 82.6, 62.3, 54.5, 53.8, 49.6, 46.1, 44.3, 43.7, 37.3, 29.7, 27.8, 24.1, 21.4.

### EXAMPLE 5

### Synthesis of N-(Toluene-4-sulfonyl)-L-prolyl-L-4-(4-methylpiperazin-1-ylcarbonyloxy)phenylalanine

The title compound was prepared from the product of Example 1 using the procedure described in Method 7.

NMR data was as follows:

¹H NMR (CD₃OD): δ=7.74 (d, 2H), 7.42 (d, 2H), 7.26 (d, 2H), 7.04 (d, 2H), 4.58-4.54 (m, 1H), 4.16-4.12 (m, 1H), 3.70 (bs, 2H) 3.53 (bs, 2H), 3.43-3.31 (m, 1H), 3.26-3.13 (m, 7H), 2.82 (s, 3H), 2.43 (s, 3H), 1.98-1.94 (m, 1H), 1.76-1.51 (m, 3H).

¹³C NMR (CD₃OD): δ=175.7, 173.6, 154.8, 151.6, 146.1, 136.3, 134.8, 131.9, 131.3, 129.1, 122.7, 63.6, 55.9, 53.9, 50.7, 43.5, 37.6, 31.3, 25.5, 21.5.

### EXAMPLE 6

### Synthesis of N-(Toluene-4-sulfonyl)-L-prolyl-L-4-(N,N-dimethylcarbamyloxy)phenylalanine n-Butyl Ester

The title compound was prepared following the procedure outlined for the preparation of Example 2 and substitution of appropriate starting materials.

NMR data was as follows:

¹H NMR (CD₃)₂SO: δ=8.31 (d, 1H), 7.70 (d, 2H), 7.41 (d, 2H), 7.23 (d, 2H), 6.99 (d, 2H), 4.53-4.46 (m, 1H), 4.10-4.01 (m, 1H), 3.63-3.30 (m, 1H), 3.10-2.96 (m, 3H), 3.00 (s, 3H), 2.88 (s, 3H), 2.39 (s, 3H), 1.59-1.30 (m, 6H), 1.33-1.20 (m, 2H), 0.85 (t, 3H).

¹³C NMR (CD₃)₂SO: δ=171.4, 171.3, 154.2, 150.2, 143.7, 134.0, 130.1, 130.0, 127.6, 121.7, 64.3, 61.2, 59.2, 53.4, 49.0, 36.2, 36.0, 35.8, 30.0, 23.8, 21.0, 18.5, 13.5.

### EXAMPLE 7

### Synthesis of N-(Toluene-4-sulfonyl)-L-prolyl-L-4-(N,N-dimethylcarbamyloxy)phenylalanine Cyclopentyl Ester

The title compound was prepared following the procedure outlined for the preparation of Example 2 and substitution of appropriate starting materials.

NMR data was as follows:

¹H NMR (CD₃)₂SO: δ=8.27 (d, 1H), 7.70 (d, 2H), 7.41 (d, 2H), 7.22 (d, 2H), 6.99 (d, 2H), 5.04 (bs, 1H), 4.48-4.40 (m, 1H), 4.08-4.05 (m, 1H), 3.34-3.30 (m, 1H), 3.09-2.95 (m, 3H), 3.00 (s, 3H), 2.88 (s, 3H), 2.39 (s, 3H), 1.76-1.74 (m, 2H), 1.57-1.40 (m, 10H).

¹³C NMR (CD₃)₂SO: δ=171.3, 171.0, 154.2, 150.2, 432.7, 134.1, 130.1, 130.0, 127.6, 121.6, 77.4, 61.2, 53.4, 49.0, 36.2, 36.1, 35.7, 32.0, 30.5, 23.8, 23.2, 21.0.

### EXAMPLE 8

### Synthesis of N-(Toluene-4-sulfonyl)-L-prolyl-L-4-(N,N-dimethylcarbamyloxy)phenylalanine tert-Butyl Ester

The title compound was prepared following the procedure outlined for the preparation of Example 2 and substitution of appropriate starting materials.

NMR data was as follows:

¹H NMR (CD₃)₂SO: δ=8.18 (d, 1H), 7.71 (d, 2H), 7.41 (d, 2H), 7.23 (d, 2H), 6.99 (d, 2H), 4.42-4.38 (m, 1H), 4.10-4.07 (m, 1H), 3.37-3.30 (m, 1H), 3.09-2.95 (m, 3H), 3.00 (s, 3H), 2.88 (s, 3H), 2.39 (s, 3H), 1.58-1.50 (m, 3H), 1.40-1.30 (m, 1H), 1.36 (s, 9H).

¹³C NMR (CD₃)₂SO: δ=171.1, 170.3, 154.2, 150.2, 143.8, 134.2, 134.1, 130.2, 130.0, 127.6, 121.6, 81.0, 61.3, 53.8, 49.0, 36.3, 36.0, 35.9, 30.5, 27.5, 23.8, 21.0.

### EXAMPLE 9

### Synthesis of N-(Toluene-4-sulfonyl)-L-prolyl-L-4-(N,N-dimethylcarbamyloxy)phenylalanine

The title compound was prepared from the product of Example 2 using the procedure described in Method 7.

NMR data was as follows:

¹H NMR (CD₃)₂SO: δ=8.13 (d, 1H), 7.70 (d, 2H), 7.41 (d, 2H), 7.23 (d, 2H), 6.99 (d, 2H), 4.51-4.44 (m, 1H), 4.11-4.09 (m, 1H), 3.40-3.34 (m, 2H), 3.11-2.94 (m, 3H), 3.00 (s, 3H), 2.87 (s, 3H), 2.39 (s, 3H), 1.59-1.36 (m, 4H).

¹³C NMR (CD₃)₂SO: δ=172.7, 171.2, 153.6, 150.2, 143.8, 134.3, 134.0, 130.2, 130.0, 127.6, 121.6, 61.3, 53.2, 49.0, 36.3, 36.1, 35.9, 30.4, 23.8, 21.0.

### EXAMPLE 10

### Synthesis of N-(Toluene-4-sulfonyl)-L-prolyl-L-3-(N,N-dimethylcarbamyloxy)phenylalanine Ethyl Ester

The title compound was prepared following the procedure outlined for the preparation of Example 2 and substitution of appropriate starting materials.

NMR data was as follows:

¹H NMR (CDCl₃): δ =7.74 (m, 2H), 7.70-7.36 (m, 4H), 7.24-7.14 (m, 3H), 6.93-4.90 (m, 1H), 4.78-4.27 (m, 3H), 4.05-3.55 (m, 0.5H), 3.48-3.43 (m, 0.5H), 3.37-3.30 (m, 3H), 3.02-3.08 (bs, 3H), 2.99 (bs, 3H), 2.45 (s, 1.5H), 2.43 (s, 1.5H), 2.12 (m, 1H), 198, 1.80 (m, 0.5M), 1.62-1.44 (m, 2.5H), 1.29 (t, 1.5H), 1.24 (t, 1.5H).

¹³C NMR (CDCl₃): δ =171.1, 171.0, 170.9, 154.9, 154.8, 151.8, 151.6, 144.4, 144.3, 137.6, 137.1, 133.1, 132.9, 130.0, 129.9, 129.5, 129.2, 127.9, 127.9, 126.5, 126.1, 122.9, 122.7, 120.7, 120.5.

### EXAMPLE 11

### Synthesis of N-(Toluene-4-sulfonyl)-L-(5,5-dimethyl)thiaprolyl-L-4-(N,N-dimethylcarbamyloxy)phenylalanine Isopropyl Ester

The title compound was prepared following the procedure outlined for the preparation of Example 2 and substitution of appropriate starting materials.

NMR data was as follows:

¹H NMR (CDCl₃): δ =7.76 (d, 2H), 7.35 (d, 2H), 7.22 (d, 2H), 7.01 (m, 3H), 5.05 (m, 1H), 4.85 (m, 1H), 4.57 (d, 1H), 4.38 (d, 1H), 3.86 (s, 1H), 3.19-3.00 (m, 2H), 3.09 (s, 3H), 3.01 (s, 3H), 2.45 (s, 3H), 1.24 (t, 6H), 1.16 (s, 3H), 1.09 (s, 3H).

¹³C NMR (CDCl₃): δ =170.3, 168.4, 154.9, 150.6, 144.8, 132.9, 132.8, 130.3, 130.0, 128.2, 121.7, 73.4, 69.5, 54.5, 53.2, 50.4, 37.7, 36.5, 36.3, 29.0, 23.8, 21.5, 21.4.

### EXAMPLE 12

### Synthesis of N-(Toluene-4-sulfonyl)-L-(5,5-dimethyl)thiaprolyl-L-4-(N,N-dimethylcarbamyloxy)phenylalanine tert-Butyl Ester

The title compound was prepared following the procedure outlined for the preparation of Example 2 and substitution of appropriate starting materials.

NMR data was as follows:

¹H NMR (CDCl₃): δ =7.75 (d, 2H), 7.34 (d, 2H), 7.23 (d, 2H), 7.05-6.98 (m, 3H), 4.76 (m, 1H), 4.56 (d, 1H), 4.40 (d, 1H), 3.85 (s, 1H), 3.09-3.00 (m, 8H), 2.44 (s, 314), 1.43 (s, 3H), 1.16 (s, 3H), 1.09 (s, 3H).

¹³C NMR (CDCl₃): δ =169.8, 168.3, 154.9, 150.6, 144.8, 133.2, 132.9, 130.4, 130.0, 128.2, 121.6, 82.6, 73.4, 54.6, 53.8, 50.4, 37.8, 36.5, 36.3, 29.0, 27.7, 23.8, 21.5.

### EXAMPLE 13

### Synthesis of N-(Toluene-4-sulfonyl)-L-(5,5-dimethyl)thiaprolyl-L-4-(N,N-dimethylcarbamyloxy)phenylalanine

The title compound was prepared from the product of Example 11 using the procedure described in Method 7.

NMR data was as follows:

¹H NMR (CDCl₃): δ =7.76 (d, 2H), 7.35 (d, 2H), 7.25 (d, 2H), 7.14 (d, 1H), 7.02 (d, 2H), 5.17 (br s, 1H), 4.89 (m, 1H), 4.56 (d, 1H), 4.40 (d, 1H), 3.90 (s, 1H), 3.30-3.00 (m, 8H), 2.43 (s, 3H), 1.09 (s, 6H).

¹³C NMR (CDCl₃): δ =172.7, 169.3, 155.2, 150.6, 144.9, 133.1, 132.7, 130.5, 130.1, 128.1, 121.9, 73.3, 54.5, 53.3, 50.5, 36.9, 36.6, 36.4, 29.0, 23.7, 21.5.

### EXAMPLE 14

### Synthesis of N-(Toluene-4-sulfonyl)-L-[(1,1-dioxo)thiamorpholin-3-carbonyl]-L-4-(N,N-dimethylcarbamyloxy)phenylalanine tert-Butyl Ester

L-Thiamorpholine-5-carboxylic acid was prepared by the method of Larsson and Carlson (Acta Chemica Scan. 1994, 48, 517-525). N-(Toluene-4-sulfonyl)-L-thiamorpholine-5-carboxylic acid was prepared using the procedure described in Method 1 and was then coupled to t-butyl tyrosine in DMF in the presence of BOP and NMM, to give after aqueous workup and flash chromatography N-(Toluene-4-sulfonyl)-L-[thiamorpholin-3-carbonyl]-L-4-phenylalanine tert-butyl ester.

Formation of the 4-(N,N-dimethylcarbamyloxy) group was per Example 2 above and oxidation of the thiamorpholino group to the 1,1-dioxo-thiamorpholino group was per Larsson and Carlson (Acta Chemica Scan. 1994, 48, 522).

NMR data was as follows:

¹H NMR (CDCl₃): δ =7.68 (d, 2H), 7.37 (d, 2H), 7.08 (m, 4H), 6.73 (d, 1H), 5.11 (m, 1H), 4.62 (m, 1H), 4.23 (m, 1H), 4.00 (m, 1H), 3.82 (m, 1H), 3.14 (s, 3H), 3.03 (s, 3H), 2.80 (m, 5H), 2.44 (s, 3H), 1.48 (s, 9H).

¹³C NMR (CDCl₃): δ =171.3, 169.9, 164.4, 145.6, 135.4, 132.6, 130.8, 130.4, 127.3, 121.9, 83.0, 56.1, 53.8, 49.4, 48.7, 44.5, 42.0, 36.9, 36.6, 36.4, 27.8, 21.5.

### EXAMPLE 15

### Synthesis of N-(Toluene-4-sulfonyl)-L-[(1,1-dioxo)thiamorpholin-3-carbonyl]-L-4-(N,N-dimethylcarbamyloxy)phenylalanine

The title compound was prepared from the product of Example 14 using the procedure described in Method 11.

NMR data was as follows:

¹H NMR (CD₃OD): δ=7.77 (d, 2H), 7.40 (d, 2H), 7.22 (d, 2H), 7.00 (d, 2H), 5.19 (m, 1H), 4.65 (m, 1H), 4.30 (m, 1H), 3.95 (m, 1H), 3.61 (m, 1H), 3.20 (m, 5H), 3.09 (s, 3H), 2.97 (s, 3H), 2.43 (s, 3H).

¹³C NMR (CD₃OD): δ=174.1, 168.0, 157.0, 152.0, 146.4, 137.7, 135.3, 131.7, 131.6, 128.8, 123.0, 57.1, 54.8, 51.1, 50.9, 48.0, 47.7, 43.2, 37.4, 36.8, 36.7, 21.5.

### EXAMPLE 16

### Synthesis of N-(Toluene-4-sulfonyl)-L-(5,5-dimethyl)thiaprolyl-L-4-(4-methylpiperazin-1-ylcarbonyloxy)phenylalanine tert-Butyl Ester

The title compound was prepared following the procedure outlined for the preparation of Example 4 and substitution of appropriate starting materials.

NMR data was as follows:

¹H NMR (CDCl₃): δ =7.74 (d, 2H), 7.33 (d, 2H), 7.25 (d, 2H), 7.20-7.00 (m, 3H), 4.74 (m, 1H), 4.55 (d, 1H), 4.38 (d, 1H), 3.83 (s, 1H), 3.66 (br m, 2H), 3.57 (br m, 2H), 3.08-3.05 (m, 2H), 2.45-2.42 (m, 7H), 2.33 (s, 3H), 1.42 (s, 9H), 1.15 (s, 3H), 1.08 (s, 3H).

¹³C NMR (CDCl₃): δ=169.7, 168.2, 153.6, 150.3, 144.7, 133.3, 132.7, 130.4, 129.9, 128.1, 121.5, 82.6, 73.4, 54.5, 53.7, 50.4, 46.0, 44.2, 43.6, 37.7, 28.9, 27.7, 23.8, 21.4.

### EXAMPLE 17

### Synthesis of N-(Toluene-4-sulfonyl)-L-(5,5-dimethyl)thiaprolyl-L-4-(4-methylpiperazin-1-ylcarbonyloxy)phenylalanine

The title compound was prepared from the product Example 16 using the procedure described in Method 11.

NMR data was as follows:

¹H NMR (CDCl₃): δ=8.31 (d, 1H), 7.72 (d, 2H), 7.42-7.35 (m, 4H), 7.08 (d, 2H), 4.90-4.68 (m, 1H), 4.64-4.61 (m, 1H), 4.47-4.44 (m, 1H), 4.01 (s, 1H), 3.36-3.32 (br m, 4H), 3.27-3.25 (m, 1H), 3.22-3.10 (m, 1H), 2.94 (s, 3H), 2.43 (s, 3H), 1.14 (s, 3H), 1.07 (s, 3H).

### EXAMPLE 18

### Synthesis of N-(Toluene-4-sulfonyl)sarcosyl-L-4-(N,N-dimethylcarbamyloxy)phenylalanine Isopropyl Ester

The title compound was prepared following the procedure outlined for the preparation of Example 2 and substitution of appropriate starting materials.

NMR data was as follows:

¹H NMR (CDCl₃): δ =7.66 (d, 2H), 7.34 (d, 2H), 7.18 (d, 2H), 7.07 (d, 2H), 6.98 (d, 1H), 5.03 (m, 1H), 4.81 (m, 1H), 3.69 (d, 1H), 3.49 (d, 1H), 3.08 (m, 2H), 3.04 (s, 3H), 2.99 (s, 3H), 2.63 (s, 3H), 2.43 (s, 3H).

¹³C NMR (CDCl₃): δ =167.4, 154.9, 150.8, 144.4, 132.6, 130.2, 130.1, 127.7, 122.0, 110.9, 69.5, 57.3, 53.9, 53.0, 37.1, 36.6, 21.6, 21.4.

### EXAMPLE 19

### Synthesis of N-(Toluene-4-sulfonyl)sarcosyl-L-4-(N,N-dimethylcarbamyloxy)phenylalanine tert-Butyl Ester

The title compound was prepared following the procedure for the preparation of Example 2 and substitution of appropriate starting materials.

NMR data was as follows:

¹H NMR (CDCl₃): δ =7.67 (d, 2H), 7.34 (d, 2H), 7.19 (d, 2H), 7.03 (d, 2H), 6.98 (d, 1H), 4.76 (m, 1H), 3.67 (q, 1H), 3.06 (m, 2H), 3.16 (s, 3H), 2.99 (s, 3H), 2.64 (s, 3H), 2.43 (s, 3H), 1.42 (s, 9H).

¹³C NMR (CDCl₃): δ =170.0, 137.2, 154.9, 150.7, 144.3, 133.2, 132.9, 130.3, 130.0, 127.7, 121.9, 82.6, 83.9, 53.3, 37.2, 36.6, 36.4, 27.9, 21.4.

### EXAMPLE 20

### Synthesis of N-(Toluene-4-sulfonyl)sarcosyl-L-4-(N,N-dimethylcarbamyloxy)phenylalanine

The title compound was prepared from the product of Example 18 using the procedure described in Method 7.

NMR data was as follows:

¹H NMR (CDCl₃): δ =7.41 (d, 2H), 7.10 (d, 2H), 6.98 (d, 2H), 6.75 (d, 2H), 4.42 (m, 1H), 3.43 (m, 2H), 3.04 (m, 2H), 2.80 (s, 3H), 2.69 (s, 3H), 2.33 (s, 3H), 2.14 (s, 3H).

¹³C NMR (CDCl₃): δ =174.2, 170.2, 156.9, 151.9, 145.6, 135.5, 135.2, 131.4, 131.1, 128.9, 123.0, 54.6, 54.0, 37.4, 36.8, 36.7, 21.4.

### EXAMPLE 21

### Synthesis of N-(Toluene-4-sulfonyl)-L-prolyl-L-4-(N,N-dimethylaminosulfonyloxy)phenylalanine tert-Butyl Ester

Substituting dimethysulfamoyl chloride for dimethylcarbamyl chloride, and following the method for the preparation of Example 2, gave the title compound.

NMR data was as follows:

¹H NMR (CDCl₃): δ =7.72 (d, 2H), 7.34 (d, 2H), 7.21 (s, 4H), 4.69 (m, 1H), 4.04 (m, 1H), 3.4 (m, 1H), 3.24 (m, 3H), 2.96 (s, 6H), 2.42 (s, 3H), 2.02 (m, 1H), 1.45 (m, 13H).

¹³C NMR (CDCl₃): δ =166.3, 165.3, 144.8, 140.0, 130.9, 126.4, 125.6, 123.5, 117.3, 95.5, 78.3, 57.8, 49.2, 45.2, 34.2, 32.9, 25.0, 23.4, 19.7, 17.1.

### EXAMPLE 22

### Synthesis of N-(Toluene-4-sulfonyl)-L-prolyl-L-4-(N,N-dimethylaminosulfonyloxy)phenylalanine

The title compound was prepared from the product of Example 21 using the procedure described in method 11.

NMR data was as follows:

¹H NMR (CD₃OD): δ=7.73 (d, 2H), 7.41 (d, 2H), 7.38 (d, 2H), 7.22 (d, 2H), 4.69 (m, 1H), 4.11 (m, 1H), 3.41 (m, 2H), 3.19 (m, 2H), 2.94 (s, 6H), 2.41 (s, 3A), 1.78 (m, 1H), 1.61 (m, 3H).

¹³C NMR (CD₃OD): δ=174.3, 174.0, 150.8, 145.9, 137.3, 135.1, 132.1, 131.2, 129.1, 123.1, 63.3, 54.6, 50.6, 39.1, 37.5, 31.6, 25.3, 21.5.

### EXAMPLE 23

### Synthesis of N-(Toluene-4-sulfonyl)-sarcosyl-L-(4-morpholinecarbamyloxy) phenylalanine t-butyl ester

Substituting sacrosine for L-proline in the preparation of Ts-Pro-Tyr(H)-O-t-butyl ester and substitution of 4-morpholinecarbonyl chloride for dimethylcarbamyl chloride, and following the method for the preparation of Example 2, gave the title compound as a white solid.

NMR data was as follows:

¹H NMR (CDCl₃): δ 7.61 (d, 2H), 7.28 (d, 2H), 7.16 (d, 2H), 7.02 (d, 2H), 4.69 (m, 1H), 3.67 (m, 8H), 3.58 (m, 1H), 3.48 (m, 1H), 3.06 (m, 2H), 2.59 (s, 3H), 2.36 (s, 3H), 1.26 (s, 9H).

¹³C NMR (CDCl₃): δ 169.7, 167.1, 153.5, 150.1, 144.1, 133.1, 133.0, 133.0, 130.1, 129.8, 127.4, 121.6, 82.6, 66.3, 53.6, 53.1, 44.5, 43.7, 36.9, 36.4, 27.6, 21.2.

### EXAMPLE 24

### Synthesis of N-(Toluene-4-sulfonyl)sarcosyl-L-4-(isonipecotoyloxy)phenylalanine

The title compound was prepared from the product of Example 23 using the procedure described in Method 11.

NMR data was as follows:

¹H NMR (CD₃OD): δ=7.30 (d, 2H), 7.02 (d, 2H), 6.88 (d, 2H), 6.67 (d, 2H), 4.33 (m, 1H), 3.32 (m, 3H), 3.25 (m, 2H), 3.12 (m, 3H), 2.89 (m, 1H), 2.70 (m, 3H), 2.22 (s, 3H), 2.03 (s, 3H).

¹³C NMR (CD₃OD): δ=174.2, 170.3, 155.6, 151.7, 145.6, 135.8, 135.2, 131.5, 131.1, 128.9, 123.0, 67.5, 54.6, 54.0, 37.4, 36.8, 21.5.

### EXAMPLE 25

### Synthesis of N-(Toluene-4-sulfonyl)-L-[(1,1-dioxo)thiamorpholin-3-carbonyl]-L-4-(morpholin-4-ylcarbonyloxy)phenylalanine tert-Butyl Ester

Substitution of 4-morpholinecarbonyl chloride for dimethylcarbamyl chloride, and following the methods for the preparation of Example 2 and 14, gave the title compound as a white solid.

NMR data was as follows:

¹H NMR (CDCl₃): δ =7.76 (d, 1H), 7.68 (d, 1H), 7.37 (m, 2H), 7.14 (m, 2H), 7.05 (m, 1H), 6.97 (d, 1H), 6.80 (d, 0.5H), 6.57 (d, 0.5H), 5.09 (m, 0.5H), 4.91 (m, 0.5H), 4.75 (m, 0.5H), 4.62 (m, 0.5H), 4.25 (m, 0.5H), 4.09 (m, 2H), 3.79 (m, 4H), 3.65 (m, 4H), 2.91 (s, 3H), 2.44 (s, 3H), 1.69 (s, 4H), 1.44 (s, 5H).

¹³C NMR (CDCl₃): δ=170.0, 169.8, 164.8, 164.4, 153.7, 150.4, 145.6, 145.4, 135.4, 135.3, 132.9, 130.8, 130.7, 130.5, 130.4, 127.5, 127.2, 122.1, 121.8, 83.01, 82.8, 66.4, 56.1, 56.1, 53.7, 53.6, 49.5, 49.3, 48.6, 44.7, 43.9, 42.0, 41.6, 36.9, 36.3, 27.8, 21.5.

### EXAMPLE 26

### Synthesis of N-(Toluene-4-sulfonyl)-L-[(1,1-dioxo)thiamorpholin-3-carbonyl]-L-4-(morpholin-4-ylcarbonyloxy)phenylalanine

The title compound was prepared from the product of Example 25 using the procedure described in Method 11.

NMR data was as follows:

¹H NMR (CD₃OD): δ=7.67 (m, 2H), 7.32 (m, 2H), 7.08 (m, 2H), 6.93 (m, 2H), 5.09 (m, 1H), 4.54 (m, 1H), 4.19 (m, 0.5H), 4.02 (m, 0.5H), 3.81 (m, 0.5H), 3.66 (m, 8H), 2.99 (m, 7H), 2.32 (s, 3H).

¹³C NMR (CD₃OD): δ=174.0, 168.0, 155.7, 151.9, 151.8, 146.6, 146.4, 137.5, 135.5, 135.3, 131.7, 131.6, 131.6, 128.8, 123.3, 122.9, 67.6, 57.3, 57.1, 54.8, 51.1, 50.9, 50.6, 46.0, 45.3, 45.2, 43.0, 37.4, 37.0, 21.5.

### EXAMPLE 27

### Synthesis of N-(4-Fluorobenzenesulfonyl)-L-prolyl-L-4-(N,N-dimethylcarbamyloxy)phenylalanine tert-Butyl Ester

The title compound was prepared following the procedure outlined for the preparation of Example 2 and substitution of appropriate starting materials.

NMR data was as follows:

¹³C NMR (CDCl₃): δ =7.87-7.83 (m, 2H), 7.26-7.13 (m, 5H), 4.74-4.69 (m, 1H), 4.05 (m, 1H), 3.36 (m, 1H), 3.24-3.17 (m, 1H), 3.11-3.01 (m, 4H), 2.97 (s, 3H), 2.05-2.02 (m, 1H), 1.60-1.47 (m, 3H), 1.46 (s, 9H).

¹³C NMR (CDCl₃): δ =170.6, 170.0, 165.7, 154.9, 150.6, 133.2, 132.4, 130.7, 130.2, 121.7, 116.7, 82.7, 62.3, 53.7, 49.6, 37.2, 36.6, 36.4, 29.9, 27.9, 24.2.

### EXAMPLE 28

### Synthesis of N-(Toluene-4-sulfonyl)sarcosyl-L-4-(4-methylpiperazin-1-ylcarbonyloxy)phenylalanine tert-Butyl Ester

The title compound was prepared following the procedure outlined for the preparation of Example 4 and substitution of appropriate starting materials.

NMR data was as follows:

¹H NMR (CDCl₃): δ =8.17 (d, 1H), 7.59 (d, 2H), 7.26 (d, 2H), 7.13 (d, 2H), 7.00 (d, 2H), 4.66 (m, 1H), 3.60 (m, 6H), 3.04 (m, 2H), 2.56 (s, 3H), 2.40 (m, 7H), 2.34 (s, 3H), 1.41 (s, 9H).

¹³C NMR (CDCl₃): δ =169.7, 167.0, 153.4, 150.2, 144.0, 133.0, 132.9, 130.1, 129.8, 127.4, 121.6, 82.2, 54.3, 53.5, 53.1, 45.8, 44.2, 43.5, 36.9, 27.6, 21.2.

### EXAMPLE 29

### Synthesis of N-(Toluene-4-sulfonyl)-L-(1,1-dioxo-5,5-dimethyl)-thiaprolyl-L-4-(N,N-dimethylcarbamyloxy)phenylalanine tert-Butyl Ester

The product of Example 12 was oxidized by the method of Larsson and Carlson (Acta Chemica Scan. 1994, 48, 517-525), yielding the title compound as a white solid.

NMR data was as follows:

¹H NMR (CDCl₃): δ =7.73 (d, 2H), 7.36 (d, 2H), 7.21 (d, 2H), 7.06-6.95 (m, 3H), 4.79 (m, 1H), 4.38 (dd, 2H), 4.10 (s, 1H), 3.18-2.95 (m, 8H), 2.43 (s, 3H), 1.45 (s, 9H), 1.33 (s, 3H), 1.08 (s, 3H).

¹³C NMR (CDCl₃): δ =169.8, 166.2, 154.9, 120.7, 145.8, 133.0, 131.9, 130.2, 128.5, 121.9, 82.9, 68.0, 60.9, 59.3, 53.9, 37.5, 36.6, 36.3, 27.7, 21.6, 19.3, 18.5.

### EXAMPLE 30

### Synthesis of N-(1-Methylimidazolyl-4-sulfonyl)-L-prolyl-L-4-(N,N-dimethylcarbamyloxy)phenylalanine

The title compound was prepared from the product of Example 106 using the procedure described in Method 11.

NMR data was as follows:

¹H NMR (CDCl₃): δ =8.07 (d, 1H), 7.75 (s, 1H), 7.71 (s, 1H), 7.25 (d, 2H), 7.01 (d, 2H), 4.71-4.66 (m, 1H), 4.28-4.24 (m, 1H), 3.77 (s, 3H), 3.42-3.05 (m, 3H), 3.09 (s, 3H), 2.96 (s, 3H), 1.84-1.69 (m, 2H), 1.61-1.54 (m, 2H).

¹³C NMR (CDCl₃): δ =174.4, 174.1, 156.9, 151.9, 141.8, 137.7, 135.6, 131.6, 127.6, 122.9, 63.7, 54.7, 50.8, 37.4, 36.8, 36.7, 34.3, 31.6, 25.4.

### Preparative Example A

### Synthesis of 2-(Saccharin-2-yl)propionoyl-L-4-(N,N-dimethylcarbamyloxy)phenylalanine tert-Butyl Ester

N-(Benzisothiazolone)-L-alanyl-L-tyrosine t-butyl ester was prepared by first combining sodium hydride (washed free of mineral oil) in THF chilled to 0° C., and a solution of N-(2-methoxycarbonyl)sulfonyl-L-alanine-L-tyrosine t-butyl ester in THF which was added dropwise. The reaction was stirred at 0° C. for one hour and then at room temperature for two hours. The reaction mixture was extracted with EtOAc and 0.2 N HCl, the combined EtOAc layers were washed successively with 0.2 N HCl, sat. NaHCO₃, and sat. NaCl. The organic layer was dried over MgSO₄, filtered and concentrated. The residue was filtered by silica gel chromatography to afford N-(benzisothiazolone)-L-alanyl-L-tyrosine t-butyl ester.

The title compound was then prepared following the procedure described in Example 2.

NMR data was as follows:

¹H NMR (DMSO-d₆, 400 MHz) (1:1 mixture of diastereomers) δ=8.15 (m, 2H); 8.5 (m, 3H); 7.20 (m, 2H); 6.95 (m, 2H); 4.75 (m, 1H); 4.30 (m, 1H); 3.05 (s, 3H); 2.95 (m, 2H); 2.90 (s, 3H); 1.75 and 1.65 (two d, 3H); 1.30 and 1.35 (two s, 9H).

### EXAMPLE 31

### Synthesis of N-(Toluene-4-sulfonyl)-L-(1,1-dioxo-5,5-dimethyl)-thiaprolyl-L-4-(N,N-dimethylcarbamyloxy)phenylalanine

The title compound was prepared from the product of Example 29 using the procedure described in Method 11.

NMR data was as follows:

¹H NMR (CDCl₃): δ =7.75 (m, 3H), 7.29 (m, 4H), 7.08 (d, 2H), 4.95 (m, 1H), 4.46-4.20 (m, 3H), 3.17 (s, 3H), 3.30-3.10 (m, 2H), 3.02 (s, 3H), 2.43 (s, 3H), 1.15 (s, 3H), 0.88 (s, 3H).

¹³C NMR (CDCl₃): δ =127.2, 167.5, 155.8, 150.3, 145.4, 133.6, 132.6, 130.8, 130.2, 128.3, 121.9, 67.9, 65.8, 60.8, 53.9, 36.8, 36.6, 35.8, 21.6, 18.8, 15.0.

### EXAMPLE 32

### Synthesis of N-(4-Fluorobenzenesulfonyl)-L-prolyl-L-4-(N,N-dimethylcarbamyloxy)phenylalanine

The title compound was prepared from the product of Example 27 using the procedure described in Method 11.

NMR data was as follows:

¹³C NMR (CDCl₃): δ =7.88-7.84 (m, 2H), 7.54 (d, 1H), 7.26-7.18 (m, 4H), 7.01 (d, 2H), 6.92 (s, 3H), 4.88-4.83 (m, 1H), 4.14-4.11 (m, 1H), 3.39-3.29 (m, 2H), 3.13 (m, 2H), 3.00 (s, 3H), 2.99 (s, 3H), 1.92-1.89 (m, 1H), 1.59-1.43 (m, 3H).

¹³C NMR (CDCl₃): δ =173.1, 172.4, 165.6, 155.5, 150.4, 133.2, 131.9, 130.6, 130.3, 121.8, 116.6, 61.9, 53.1, 49.6, 36.6, 36.3, 30.2, 23.9.

### EXAMPLE 33

### Synthesis of N-(Toluene-4-sulfonyl)-D-prolyl-L-4-(4-methylpiperazin-1-yl)phenylalanine t-butyl Ester

The title compound was prepared following the procedure outlined for the preparation of Example 4 and substitution of appropriate starting materials.

NMR data was as follows:

¹³C NMR (CDCl₃): δ =7.72 (d, 2H), 7.33 (d, 3H), 7.17 (d, 2H), 7.02 (d, 2H), 4.71 (q, 1H), 4.09-4.06 (m, 1H), 3.67 (bs, 2H), 3.57 (bs, 2H), 3.41-3.34 (m, 1H), 3.22 (dd, 1H), 3.16-3.09 (m, 1H), 3.03 (dd, 1H), 2.46-2.43 (m, 7H), 2.05-2.02 (m, 1H), 1.57-1.43 (m, 3H), 1.47 (s, 9H).

¹³C NMR (CDCl₃): δ =170.8, 169.9, 153.6, 150.4, 144.3, 133.4, 133.1, 130.3, 130.0, 127.9, 121.6, 82.6, 62.3, 54.5, 53.8, 49.6, 46.1, 44.3, 43.7, 37.3, 29.7, 27.8, 24.1, 21.4.

### EXAMPLE 34

### Synthesis of N-(Toluene-4-sulfonyl)-N-methyl-L-alanyl-L-4-(4-methylpiperazin-1-ylcarbonyloxy)phenylalanine t-butyl ester

The title compound was prepared following the procedure outlined for the preparation of Example 4 and substitution of appropriate starting materials.

NMR data was as follows:

¹H NMR (CDCl₃): δ =7.68 (d, 2H), 7.31 (d, 2H), 7.17 (d, 2H), 7.04 (d, 2H), 6.86 (d, 1H), 4.65 (m, 1H), 4.47 (q, 1H), 3.71-3.53 (m, 4H), 3.24-2.92 (m, 2H), 2.50-2.40 (m, 10H), 2.35 (s, 3H), 1.45 (s, 9H), 0.92 (d, 3H).

¹³C NMR (CDCl₃): δ =170.1, 169.9, 153.6, 150.4, 143.9, 135.6, 133.3, 130.2, 129.9, 127.2, 121.8, 82.4, 55.4, 54.6, 53.6, 46.0, 44.2, 43.7, 37.2, 29.6, 27.8, 21.4, 11.5.

### EXAMPLE 35

### Synthesis of N-(4-Nitrobenzenesulfonyl)-L-prolyl-L-4-(N,N-dimethylcarbarnyloxy)phenylalanine tert-Butyl Ester

The title compound was prepared following the procedure outlined for the preparation of Example 2 and substitution of appropriate starting materials.

NMR data was as follows:

¹H NMR (CDCl₃): δ =8.38-8.34 (m, 2H), 8.05-8.00 (m, 2H), 7.16-2.12 (m, 2H), 7.03-6.94 (m, 3H), 4.74-4.68 (m, 1H), 4.15-4.14 (m, 1H), 3.41-3.32 (m, 1H), 3.23-3.14 (m, 2H), 3.08 (s, 3H), 3.03 (m, 1H), 2.98 (s, 3H), 2.05 (m, 1H), 1.66-1.48 (m, 3H), 1.47 (s, 9H).

¹³C NMR (CDCl₃): δ=170.0, 169.9, 154.8, 150.6, 150.4, 142.4, 132.9, 130.2, 129.0, 124.5, 121.6, 82.7, 62.2, 53.4, 49.4, 37.0, 36.5, 36.2, 30.1, 27.7, 24.1.

### EXAMPLE 36

### Synthesis of N-(Toluene-4-sulfonyl)-L-[(1,1-dioxo)-thiamorpholin-3-carbonyl]-L-4-(N,N-dimethylaminosulfonyloxy)phenylalanine tert-Butyl Ester

The title compound was prepared using the procedure described for the preparation of Example 21 and substitution of appropriate starting materials.

NMR data was as follows:

¹H NMR (CDCl₃): δ =7.73 (d, 1H), 7.67 (d, 1H), 7.35 (m, 2H), 7.27 (m, 2H), 6.88 (d, 1H), 6.66 (d, 1H), 5.08 (m, 0.5H), 4.97 (m, 0.5H), 4.71 (m, 0.5H), 4.61 (m, 0.5H), 4.25 (m, 0.5H), 4.03 (m, 1H), 3.21-3.04 (m, 4H), 2.89 (s, 3H), 2.83 (s, 3H), 2.78 (m, 3H), 2.42 (s, 3H), 1.44 (s, 4.5H), 1.38 (s, 4.5H).

¹³C NMR (CDCl₃): δ =169.8, 169.6, 164.9, 164.5, 149.3, 149.1, 145.6, 145.4, 135.4, 135.0, 134.6, 130.9, 130.6, 130.5, 127.4, 127.2, 122.0, 121.8, 83.0, 83.0, 56.0, 53.7, 49.2, 49.1, 48.5, 41.9, 41.4, 38.6, 36.8, 36.2, 27.7, 21.5.

### EXAMPLE 37

### Synthesis of N-(Toluene-4-sulfonyl)sarcosyl-L-4-(thiomorpholin-4-ylcarbonyloxy)phenylalanine tert-Butyl Ester

Substituting thiomorpholine for N-methylpiperazine, and following the method for the preparation of Example 4, gave the title compound.

NMR data was as follows:

¹H NMR (CDCl₃): δ =7.65 (d, 2H), 7.33 (d, 2H), 7.20 (d, 2H), 7.04 (d, 2H), 4.76 (m, 1H), 3.89 (m, 4H), 3.68 (d, 1H), 3.48 (d, 1H), 3.10 (m, 2H), 2.66 (m, 7H), 2.41 (s, 3H), 1.43 (s, 9H).

¹³C NMR (CDCl₃): δ =169.9, 167.2, 153.5, 150.3, 144.3, 133.1, 130.3, 130.0, 127.6, 121.8, 82.5, 53.8, 53.3, 47.0, 36.4, 37.2, 36.6, 27.8, 27.3, 27.0, 21.4.

### EXAMPLE 38

### Synthesis of N-(Toluene-4-sulfonyl)-L-N-methylalanyl-L-4-(4-methylpiperazin-1-ylcarbonyloxy)phenylalanine

The title compound was prepared from the product of Example 34 using the procedure described in Method 11.

NMR data was as follows:

¹H NMR (CDCl₃): δ =7.65 (d, 2H), 7.34 (d, 2H), 7.27 (d, 2H), 7.09 (d, 2H), 4.64-4.50 (m, 2H), 4.48-4.23 (m, 2H), 3.60-2.96 (m, 8H), 2.92 (s, 3H), 2.55 (s, 3H), 2.40 (s, 3H), 0.93 (d, 3H).

¹³C NMR (CDCl₃): δ =174.3, 173.1, 154.9, 151.6, 145.5, 137.0, 136.1, 131.6, 131.2, 128.5, 123.1, 56.4, 54.8, 54.0, 43.8, 37.3, 30.2, 21.5, 13.2.

### EXAMPLE 39

### Synthesis of N-(Toluene-4-sulfonyl)-L-prolyl-L-4-(1,1-dioxothiomorpholin-4-ylcarbonyloxy)phenylalanine

The title compound was prepared from the product of Example 81 using the procedure described in Method 11.

NMR data was as follows:

¹H NMR (CD₃OD): δ=8.03 (m, 1H), 7.73 (d, 2H), 7.41 (d, 2H), 7.28 (d, 2H), 7.08 (d, 2H), 4.70-4.65 (m, 1H), 4.12-4.00 (m, 5H), 3.38-3.36 (m, 1H), 3.31-3.06 (m, 7H), 2.43 (s, 3H), 1.77-1.48 (m, 5H).

¹³C NMR (CD₃OD): δ=168.4, 159.1, 130.0, 129.1, 125.6, 125.1, 123.0, 116.9, 57.2, 48.8, 46.3, 44.5, 31.5, 25.6, 19.3, 15.4.

### EXAMPLE 40

### Synthesis of N-(Toluene-4-sulfonyl)-L-prolyl-L-4-(thiomorpholin-4-ylcarbonyloxy)phenylalanine

The title compound was prepared from the product of Example 82 using the procedure described in Method 11.

NMR data was as follows:

¹H NMR (CD₃OD): δ=7.73 (d, 2H), 7.41 (d, 2H), 7.27 (d, 2H), 7.04 (d, 2H), 4.68-4.65 (m, 1H), 4.10-4.07 (m, 1H), 3.90 (t, 2H), 3.77 (t, 2H), 3.38-3.11 (m, 4H), 2.66 (m, 4H), 2.43 (s, 3H), 1.80-1.48 (m, 5H).

¹³C NMR (CD₃OD): δ=168.4, 168.2, 149.4, 145.7, 139.8, 129.7, 129.0, 125.6, 125.1, 123.1, 116.9, 57.2, 48.8, 44.6, 42.1, 36.0, 31.4, 25.7, 22.1, 21.8, 19.3, 15.4.

### EXAMPLE 41

### Synthesis of N-(Toluene-4-sulfonyl)-L-prolyl-L-4-(isonipecotoyloxy)phenylalanine

The title compound was prepared from the product of Example 80 using the procedure described in Method 11.

NMR data was as follows:

¹H NMR (CD₃OD): δ=8.08 (m, 1H), 7.73 (d, 2H), 7.41 (d, 2H), 7.27 (d, 2H), 7.03 (d, 2H), 4.71 (m, 1H), 4.11-4.08 (m, 1H), 3.61 (t, 2H), 3.47-3.38 (m, 3H), 3.31-3.11 (m, 4H), 2.43 (s, 3H), 1.77-1.47 (m, 10H).

¹³C NMR (CD₃OD): δ=168.3, 168.1, 158.8, 149.6, 145.9, 139.8, 129.5, 129.0, 125.6, 125.1, 123.1, 116.9, 57.2, 48.6, 44.6, 40.6, 40.1, 36.0, 31.4, 25.7, 20.9, 20.6, 19.3.

### EXAMPLE 42

### Synthesis of N-(Toluene-4-sulfonyl)-L-prolyl-L-4-(pyrrolidin-1-ylcarbonyloxy)phenylalanine

The title compound was prepared from the product of Example 83 using the procedure described in Method 11.

NMR data was as follows:

¹H NMR (CD₃OD): δ=8.08 (m, 1H), 7.73 (d, 2H), 7.41 (d, 2H), 7.04 (d, 2H), 7.27 (d, 2H), 4.72-4.68 (m, 1H), 4.11-4.08 (m, 1H), 3.57-3.53 (t, 2H), 3.43-3.28 (m, 3H), 3.25-3.06 (m, 4H), 2.43 (s, 3H), 1.99-1.80 (m, 4H), 1.78-1.49 (m, 5H).

¹³C NMR (CD₃OD): δ=168.2, 158.3, 149.2, 145.8, 139.8, 129.4, 129.1, 125.6, 125.1, 123.1, 116.9, 57.2, 48.7, 44.5, 41.5, 31.4, 25.7, 20.6, 19.8, 19.3, 15.4.

### EXAMPLE 43

### Synthesis of N-(Toluene-4-sulfonyl)-L-prolyl-L-4-(morpholin-4-ylcarbonyloxy)phenylalanine

The title compound was prepared from the product of Example 108 using the procedure described in Method 11.

NMR data was as follows:

¹H NMR (CD₃OD): δ=7.73 (d, 2H), 7.41 (d, 2H), 7.27 (d, 2H), 7.04 (d, 2H), 4.95-4.93 (m, 1H), 4.10-4.07 (m, 1H), 3.71-3.65 (m, 6H), 3.50 (t, 2H), 3.40-3.10 (m, 4H), 2.43 (s, 3H), 1.78-1.48 (m, 4H).

¹³C NMR (CD₃OD): δ=168.4, 168.2, 149.6, 145.7, 139.8, 129.1, 125.6, 125.1, 123.1, 116.8, 61.5, 57.2, 44.5, 36.0, 31.4, 25.6, 19.3, 15.4.

### EXAMPLE 44

### Synthesis of N-(Toluene-4-sulfonyl)-L-prolyl-L-4-(4-methylpiperazin-1-ylcarbonyloxy)phenylalanine Neopentyl Ester

Titanium isopropoxide (0.3 equivalents) was added to Tos-Pro-Tyr ethyl ester (1 equivalent) and an excess of neopentyl alcohol. The mixture was heated to reflux under an argon atmosphere overnight. Excess neopentyl alcohol was removed under reduced pressure and the residue purified by flash column chromatography (silica, hexane: EtOAc 2:1) to give the neopentyl ester a white solid (0.9 g, 85%).

The title compound was prepared following the procedure described in Example 4.

NMR data was as follows:

¹H NMR (DMSO-d₆, 400 MHz) δ=8.29 (d, 1H, J=7.91 Hz); 7.68 (d, 2H, J=8.45 Hz); 7.40 (d, 2H, J=8.34 Hz); 7.24 (d, 2H, J=8.57 Hz); 7.00 (d, 2H, J=8.57 Hz); 4.56 (m, 1H); 4.07 (m, 1H); 3.73 (s, 2H); 3.55 (br s, 2H); 3.40 (m, 3H); 3.10 (m, 3H); 2.40 (s, 3H); 2.35 (br s, 4H); 2.20 (s, 3H); 1.55 (m, 3H); 1.37 (m, 1H); 0.85 (s, 9H).

### EXAMPLE 45

### Synthesis of N-(Toluene-4-sulfonyl)-L-prolyl-L-4-(N,N-dimethylcarbamyloxy)phenylalanine Neopentyl Ester

Titanium isopropoxide (0.3 equivalents) was added to Tos-Pro-Tyr ethyl ester (1 equivalent) and an excess of neopentyl alcohol. The mixture was heated to reflux under an argon atmosphere overnight. Excess neopentyl alcohol was removed under reduced pressure and the residue purified by flash column chromatography (silica, hexane: EtOAc 2:1) to give the neopentyl ester a white solid (0.9 g, 85%).

The title compound was prepared following the procedure described in Example 2.

NMR data was as follows:

¹H NMR (DMSO-d₆, 400 MHz) δ=8.28 (d, 1H, J=8.13 Hz); 7.68 (d, 2H, J=8.4 Hz); 7.40 (d, 2H, J=7.9 Hz); 7.23 (d, 2H, J=8.56 Hz), 6.99 (d, 2H, J=8.35 Hz); 4.57 (m, 3H); 2.40 (s, 3H); 1.55 (m, 3H); 1.38 (m, 1H); 0.85 (s, 9H).

### EXAMPLE 46

### Synthesis of 2-(Saccharin-2-yl)propionoyl-L-4-(4-methylpiperazin-1-ylcarbonyloxy)phenylalanine tert-Butyl Ester

The title compound was prepared following the procedure described in Preparative Example A and Example 4.

NMR data was as follows:

¹H NMR (DMSO-d₆, 400 MHz) (1:1 mixture of diastereomers) δ=8.31 (m, 1H); 8.26 (m, 1H); 8.03 (m, 3H); 7.20 (m, 2H); 7.00 (m, 2H); 4.73 (m, 1H); 4.30 (m, 1H); 3.58 (br s, 2H); 3.40 (br s, 2H); 3.02 (m, 1H); 2.95 (m, 1H); 2.35 (br s, 4H); 2.20 (s, 3H); 2.75 and 2.65 (two d, 3H); 1.35 and 1.32 (two s, 9H).

### EXAMPLE 47

### Synthesis of 2-(Saccharin-2-yl)propionoyl-L-4-(N,N-dimethylcarbamyloxy)phenylalanine

The title compound was prepared from the product of Preparative Example A using the procedure described in Method 11.

NMR data was as follows:

¹H NMR (DMSO-d₆, 400 MHz) (1:1 mixture of diastereomers) δ=12.75 (br s, 1H); 8.28 (m, 2H); 8.05 (m, 3H); 7.20 (m, 2H); 7.00 and 9.95 (two d, 2H); 4.75 (m, 1H); 4.40 (m, 1H); 3.10 (m, 1H); 3.05 (s, 3H); 2.95 (m, 1H); 2.90 (s, 3H); 2.75 and 2.60 (two d, 3H).

### EXAMPLE 48

### Synthesis of N-(Toluene-4-sulfonyl)-L-N-methylalanyl-L-4-(N,N-dimethylcarbamyloxy)phenylalanine tert-Butyl Ester

The title compound was prepared following the procedure for the synthesis of Example 2 with the substitution of appropriate starting materials.

NMR data was as follows:

¹H NMR (CDCl₃): δ =7.68 (d, 2H), 7.31 (d, 2H), 7.17 (d, 2H), 7.04 (d, 2H), 6.87 (d, 2H), 4.67 (m, 1H), 4.48 (q, 1H), 3.09 (s, 3H), 3.00 (s, 3H), 3.14-2.92 (m, 2H), 2.46 (s, 3H), 2.43 (s, 3H), 1.45 (s, 9H), 0.92 (d, 3H).

¹³C NMR (CDCl₃): δ =170.2, 169.9, 154.9, 150.6, 143.9, 135.6, 133.2, 130.2, 130.0, 127.3, 121.9, 82.5, 55.5, 53.7, 37.2, 36.6, 36.4, 29.7, 27.8, 21.4, 11.5.

### EXAMPLE 49

### Synthesis of N-(Toluene-4-sulfonyl)-L-(thiamorpholin-3-carbonyl)-L-4-(N,N-dimethylcarbamloxy)phenylalanine tert-Butyl Ester

L-Thiamorpholine-3-carboxylic acid was prepared by the method of Larsson and Carlson (Acta Chemica Scan. 1994, 48, 517-525). N-(Toluene-4-sulfonyl)-L-thiamorpholine-3-carboxylic acid was prepared using the procedure described in Method 1. The title compound was prepared following the procedure for the synthesis of Example 2 with substitution of appropriate starting materials.

NMR data was as follows:

¹H NMR (CDCl₃): δ =7.69 (d, 2H), 7.31 (d, 2H), 7.16 (d, 2H), 6.98 (d, 2H), 6.86 (d, 1H), 4.71 (m, 1H), 4.62 (m, 1H), 3.94 (m, 1H), 3.31 (m, 1H), 3.09 (m, 4H), 2.98 (s, 3H), 2.67 (m, 1H), 2.50 (m, 1H), 2.40 (s, 3H), 2.31 (m, 1H), 2.10 (m, 1H), 1.49 (s, 9H).

¹³C NMR (CDCl₃): δ =169.9, 167.4, 154.8, 150.6, 144.2, 136.8, 132.8, 130.4, 130.2, 127.3, 121.8, 82.6, 55.2, 54.0, 43.3, 36.5, 36.3, 27.8, 25.2, 24.6, 21.4.

### EXAMPLE 50

### Synthesis of N-(Toluene-4-sulfonyl)sarcosyl-L-4-(1,1-dioxothiomorpholin-4-ylcarbonyloxy)phenylalanine

The title compound was prepared from the product of Example 121 using the procedure described in Method 11.

NMR data was as follows:

¹H NMR (CD₃OD): δ=7.67 (d, 2H), 7.40 (d, 2H), 7.27 (d, 2H), 7.09 (d, 2H), 4.61 (m, 1H), 4.12 (m, 2H), 3.99 (m, 2H), 3.60 (m, 2H), 3.23 (m, 8H), 2.58 (s, 3H), 2.42 (s, 3H).

¹³C NMR (CD₃OD): δ=174.2, 170.3, 155.0, 151.6, 145.6, 136.1, 135.2, 131.5, 131.1, 128.9, 123.0, 54.6, 54.0, 52.4, 52.2, 44.4, 44.0, 37.4, 36.8, 21.4.

### EXAMPLE 51

### Synthesis of N-(Toluene-4-sulfonyl)-L-(1,1-dioxothiamorpholin-3-carbonyl)-L-4-(N,N-dimethylcarbamyloxy)phenylalanine tert-Butyl Ester

The title compound was prepared from the product of Example 49 following the procedure described by Larsson and Carlson (Acta Chemica Scan. 1994, 48, 522).

NMR data was as follows:

¹H NMR (CDCl₃): δ =7.76 (d, 2H), 7.37 (d, 2H), 7.08 (d, 2H), 6.98 (d, 2H), 6.56 (d, 1H), 4.95 (m, 1H), 4.62 (m, 1H), 3.99 (m, 2H), 3.25 (m, 1H), 3.07 (s, 3H), 2.97 (m, 8H), 2.44 (s, 3H), 1.48 (s, 9H).

¹³C NMR (CDCl₃): δ =170.0, 164.8, 154.9, 150.7, 145.4, 135.3, 132.6, 130.7, 130.3, 127.5, 122.3, 82.8, 56.1, 53.6, 49.5, 48.6, 41.6, 36.6, 36.4, 27.9, 21.6.

### EXAMPLE 52

### Synthesis of N-(Toluene-4-sulfonyl)-L-(1,1-dioxothiamorpholin-3-carbonyl)-L-4-(morpholin-4-ylcarbonyloxy)phenylalanine tert-Butyl Ester

The title compound was prepared following the procedure described for the preparation of Example 71.

NMR data was as follows:

¹H NMR (CDCl₃): δ =7.75 (d, 2H), 7.36 (d, 2H), 7.12 (d, 2H), 6.98 (d, 2H), 6.58 (d, 1H), 4.93 (m, 1H), 4.63 (m, 1H), 4.09 (m, 2H), 3.72 (m, 4H), 3.63 (m, 2H), 3.51 (m, 2H), 3.24 (m, 1H), 2.96 (m, 4H), 2.43 (s, 3H), 1.46 (s, 9H).

¹³C NMR (CDCl₃): δ =170.0, 164.8, 153.7, 150.4, 145.4, 135.2, 132.9, 130.7, 130.4, 127.5, 122.1, 82.9, 66.4, 56.1, 53.6, 49.4, 48.5, 44.7, 43.9, 41.6, 36.3, 27.8, 21.6.

### EXAMPLE 53

### Synthesis of N-(Toluene-4-sulfonyl)-L-N-methylalanyl-L-4-(N,N-dimethylcarbamyloxy)phenylalanine

The title compound was prepared from the product of Example 48 using the procedure described in Method 11.

NMR data was as follows:

¹H NMR (CDCl₃): δ =7.68 (d, 2H), 7.31 (d, 2H), 7.20 (d, 2H), 7.11-7.04 (m, 3H), 6.35 (br s, 1H), 4.81 (m, 1H), 4.52 (q, 1H), 3.35-2.98 (m, 2H), 3.09 (s, 3H), 3.00 (s, 3H), 2.45 (s, 3H), 2.43 (s, 3H), 0.91 (d, 3H).

¹³C NMR (CDCl₃): δ =173.7, 170.8, 155.2, 150.6, 144.0, 135.4, 133.2, 130.2, 130.0, 127.3, 122.1, 55.5, 53.2, 36.6, 36.5, 36.4, 29.8, 21.4, 11.6.

### EXAMPLE 54

### Synthesis of N-(4-Fluorobenzenesulfonyl)-L-(thiamorpholin-3-carbonyl)-L-4-(N,N-dimethylcarbamyloxy)phenylalanine tert-Butyl Ester

L-Thiamorpholine-3-carboxylic acid was prepared by the method of Larsson and Carlson (Acta Chemica Scan. 1994, 48, 517-525). N-(Toluene-4-sulfonyl)-L-thiamorpholine-3-carboxylic acid was prepared using the procedure described in Method 1.

The title compound was then prepared following the procedure for the synthesis of Example 2.

NMR data was as follows:

¹H NMR (CDCl₃): δ =7.87-7.82 (m, 2H), 7.20 (t, 2H), 7.16 (d, 2H), 7.00 (d, 2H), 6.76 (d, 1H), 4.74 (t, 1H), 4.65 (q, 1H), 3.92 (d, 1H), 3.32 (dd, 1H), 3.17-3.00 (m, 2H), 3.09 (s, 3H), 2.99 (s, 3H), 2.76-2.66 (m, 1H), 2.62 (dd, 1H), 2.46 (dt, 1H), 2.22 (d, 1H), 1.49 (s, 9H).

¹³C NMR (CDCl₃): δ =170.0, 167.2, 165.5, 154.8, 150.7, 135.8, 132.7, 130.5, 130.1, 121.9, 116.9, 82.8, 55.3, 53.9, 43.4, 36.6, 36.4, 36.3, 27.9, 25.8, 25.0.

### EXAMPLE 55

### Synthesis of N-(4-Fluorobenzenesulfonyl)-L-(1,1-dioxothiamorpholin-3-carbonyl)-L-4-(N,N-dimethylcarbamyloxy)phenylalanine tert-Butyl Ester

The title compound was prepared from the product of Example 54 following the procedure described by Larsson and Carlson (Acta Chemica Scan. 1994, 48, 522).

NMR data was as follows:

¹H NMR (CDCl₃): δ =7.92-7.88 (m, 2H), 7.24 (t, 2H), 7.09 (d, 2H), 6.97 (d, 2H), 6.41 (d, 1H), 4.96 (d, 1H), 4.62 (d, 1H), 4.03 (d, 1H), 3.26 (dd, 1H), 3.13-2.92 (m, 6H), 3.09 (s, 3H), 2.97 (s, 3H), 1.49 (s, 9H).

¹³C NMR (CDCl₃): δ =170.1, 165.9, 164.5, 154.9, 150.7, 134.0, 132.4, 130.5, 130.4, 122.2, 117.3, 83.0, 56.1, 53.4, 50.0, 49.1, 41.7, 36.6, 36.3, 36.1, 27.9.

### EXAMPLE 56

### Synthesis of N-(Pyridine-3-sulfonyl)-L-prolyl-L-4-(N,N-dimethylcarbamyloxy)phenylalanine tert-Butyl Ester

N-Benzyl-L-proline was coupled to L-tyrosine t-butyl ester using the procedure described in Method 12. N-Benzyl-L-prolyl-L-(N,N-dimethylcarbamyloxy)phenyl-alanine t-butyl ester was prepared following the procedure described for the preparation of Example 2. L-Prolyl-L-(4-N,N-dimethylcarbamyloxy)phenylalanine t-butyl ester was prepared from the product of the previous reaction using the procedure described in Method 4. The title compound was prepared using the procedure described for the preparation of 3-pyridine sulfonyl chloride (see Crowell et al., J. Med Chem., 1989, 32, 2436-2442) and the product of the last reaction.

NMR data was as follows:

¹H NMR (CDCl₃): δ =9.95 (d, 1H), 8.83 (dd, 1H), 8.14-8.10 (m, 1H), 7.51-7.47 (m, 1H), 7.16-7.13 (m, 3H), 7.02-6.99 (m, 2H), 4.72-4.69 (m, 1H), 4.09-4.06 (m, 1H), 3.41-3.39 (m, 1H), 3.23-3.17 (m, 1H), 3.13-2.98 (m, 1H), 3.07 (s, 3H), 2.97 (s, 3H), 2.04 (m, 1H), 1.59-1.47 (m, 3H), 1.45 (s, 9H).

¹³C NMR (CDCl₃): δ =170.1, 169.9, 154.8, 153.9, 150.5, 148.4, 135.5, 133.0, 130.1, 123.9, 121.6, 82.6, 52.2, 53.6, 49.5, 37.1, 36.5, 36.3, 29.9, 27.8, 24.0.

### Preparative Example B

### Synthesis of N-(Pyrimidine-2-sulfonyl)-L-prolyl-L-4-(N,N-dimethylcarbamyloxy)phenylalanine tert-Butyl Ester

The title compound was prepared by substituting 2-pyrimidine sulfonyl chloride (see Skulnick et al., J. Med. Chem., 1997, 40, 1149-1164) and following the method for the preparation of Example 56.

NMR data was as follows:

¹H NMR (CDCl₃): δ =8.28 (d, 2H), 7.39 (d, 1H), 7.02 (d, 2H), 6.88 (d, 2H), 6.54 (m, 1H), 4.76-4.69 (m, 1H), 4.57-4.55 (m, 1H), 3.64 (m, 1H), 3.55-3.52 (m, 1H), 3.09-3.03 (m, 1H), 3.08 (s, 3H), 2.99-2.95 (m, 1H), 2.98 (s, 3H), 2.32 (m, 1H), 2.01-1.97 (m, 3H), 1.37 (s, 9H).

¹³C NMR (CDCl₃): δ =172.1, 170.4, 160.6, 157.7, 154.8, 150.3, 133.0, 130.1, 121.3, 110.5, 82.0, 60.7, 53.3, 47.5, 37.1, 36.5, 36.3, 28.9, 27.7, 24.1.

### EXAMPLE 57

### Synthesis of N-(4-Nitrobenzenesulfonyl)-L-prolyl-L-4-(N,N-dimethylcarbamyloxy)phenylalanine

The title compound was prepared from the product of Example 35 using the procedure described in method 11.

NMR data was as follows:

¹H NMR (CDCl₃): δ =8.36 (d, 2H), 8.02 (d, 2H), 7.42 (d, 1H), 7.20 (d, 2H), 7.01 (d, 2H), 4.86 (m, 1H), 4.18-4.15 (m, 1H), 3.46-3.43 (m, 1H), 3.32-3.26 (m, 1H), 3.19-3.11 (m, 2H), 3.09 (s, 3H), 3.01 (s, 3H), 1.91 (m, 1H), 1.65-1.54 (m, 3H).

¹³C NMR (CDCl₃): δ =172.9, 171.7, 155.5, 150.4, 150.4, 142.1, 133.2, 130.5, 129.1, 124.6, 121.8, 61.9, 52.9, 49.6, 36.6, 36.3, 36.3, 30.6, 24.1.

### EXAMPLE 58

### Synthesis of N-(4—Cyanobenzenesulfonyl)-L-prolyl-L-4-(N,N-dimethylcarbamyloxy)phenylalanine tert-Butyl Ester

The title compound was prepared following the procedure described for the preparation of Example 2 and substitution of appropriate starting materials.

NMR data was as follows:

¹H NMR (CDCl₃): δ =7.94 (d, 2H), 7.82 (d, 2H), 7.13 (d, 2H), 7.05-6.99 (m, 3H), 4.71-4.66 (m, 1H), 4.12-4.09 (m, 1H), 3.36-3.35 (m, 1H), 3.22-3.11 (m, 2H), 3.07 (s, 3H), 3.06-3.01 (m, 1H), 2.97 (s, 3H), 2.05 (m, 1H), 1.63-1.37 (m, 3H), 1.46 (s, 9H).

¹³C NMR (CDCl₃): δ =170.1, 169.9, 154.8, 150.6, 140.8, 133.1, 132.9, 130.2, 128.4, 121.7, 117.1, 116.9, 82.7, 62.2, 53.4, 49.4, 37.0, 36.5, 36.3, 30.1, 27.8, 24.1.

### EXAMPLE 59

### Synthesis of N-(Toluene-4-sulfonyl)-L-(1,1-dioxothiamorpholin-3-carbonyl)-L-4-(N,N-dimethylaminosulfonyloxy)phenylalanine

The title compound was prepared from the product of Example 36 using the procedure described in Method 11.

NMR data was as follows:

¹H NMR (CD₃OD): δ=7.79 (m, 2H), 7.44 (m, 2H), 7.27 (m, 2H), 7.17 (m, 2H), 5.21 (m, 1H), 4.64 (m, 1H), 4.14 (m, 1H), 3.61 (m, 2H), 3.24 (m, 2H), 3.08 (m, 2H), 2.89 (s, 6H), 2.80 (m, 2H), 2.43 (s, 3H).

¹³C NMR (CD₃OD): δ=173.9, 168.1, 168.0, 150.8, 150.8, 146.7, 146.5, 137.6, 137.5, 137.1, 136.9, 132.2, 132.1, 131.7, 131.6, 128.8, 123.3, 123.1, 57.3, 54.8, 51.0, 50.8, 50.5, 47.9, 47.8, 43.2, 43.0, 39.0, 39.0, 37.4, 37.0, 21.5.

### EXAMPLE 60

### Synthesis of N-(Toluene-4-sulfonyl)-L-(1,1-dioxothiamorpholin-3-carbonyl)-L-4-(N,N-dimethylcarbamyloxy)phenylalanine

The title compound was prepared from the product of Example 51 using the procedure described in Method 11.

NMR data was as follows:

¹H NMR (CDCl₃): δ =7.79 (d, 2H), 7.43 (d, 2H), 7.20 (d, 2H), 7.00 (d, 2H), 5.21 (m, 1H), 4.65 (m, 1H), 4.12 (m, 1H), 3.75 (m, 1H), 3.29 (m, 3H), 3.08 (s, 3H), 3.00 (m, 1H), 3.00 (m, 1H), 2.97 (s, 3H), 2.80 (m, 3H), 2.44 (s, 3H).

¹³C NMR (CDCl₃): δ =165.1, 159.0, 147.9, 143.1, 137.6, 128.6, 126.1, 122.7, 122.6, 119.8, 114.3, 48.3, 45.8, 41.6, 34.0, 28.0, 27.8, 27.7, 12.5.

### EXAMPLE 61

### Synthesis of N-(Toluene-4-sulfonyl)-L-(1,1-dioxo)thiaprolyl-L-4-(N,N-dimethylcarbamyloxy)phenylalanine tert-Butyl Ester

The title compound was prepared from N-(toluene-4-sulfonyl)-L-thiaprolyl-L-4-(N,N-dimethylcarbamyloxy)phenylalanine tert-butyl ester, prepared as per the examples herein, following the procedure described by by Larsson and Carlson (Acta Chemica Scan. 1994, 48, 522).

NMR data was as follows:

¹H NMR (CDCl₃): δ =7.77 (d, 2H), 7.38 (d, 2H), 7.18 (m, 3H), 7.09 (d, 2H), 4.83-4.57 (m, 3.H), 3.77-3.60 (m, 2H), 3.36-3.23 (m, 1H), 3.15-3.00 (m, 7H), 2.85-2.73 (m, 1H), 2.46 (s, 3H), 1.50 (s, 9H).

### EXAMPLE 62

### Synthesis of N-(4-Fluorobenzenesulfonyl)-L-prolyl-L-4-(N,N-dimethylcarbamyloxy)phenylalanine tert-Butyl Ester

The title compound was prepared following the procedure outlined for the preparation of Example 2 and substitution of appropriate starting materials.

NMR data was as follows:

¹H NMR (CDCl₃): δ =7.96 (d, 2H), 7.80 (d, 2H), 7.26-7.13 (m, 3H), 7.01 (d, 2H), 4.72-4.70 (m, 1H), 4.11-4.08 (m, 1H), 3.40-3.37 (m, 1H), 3.25-3.10 (m, 2H), 3.07 (s, 3H), 3.04-3.02 (m, 1H), 2.98 (s, 3H), 2.06 (m, 1H), 2.06-2.04 (m, 1H), 1.61-1.52 (m, 3H), 1.46 (s, 9H).

¹³C NMR (CDCl₃): δ =170.3, 169.9, 154.9, 150.6, 139.9, 134.9, 133.1, 130.2, 128.4, 126.5, 121.7, 82.7, 62.3, 5.35, 49.6, 37.2, 36.6, 36.3, 30.0, 27.8 24.1.

### EXAMPLE 63

### Synthesis of N-(1-Methylpyrazolyl-4-sulfonyl)-L-prolyl-L-4-(N,N-dimethylcarbamyloxy)phenylalanine

The title compound was prepared from the product of Example 117 using the procedure described in Method 11.

NMR data was as follows:

¹H NMR (CDCl₃): δ =8.84 (br s, 1H), 7.93 (s, 1H), 7.79 (s, 1H), 7.68-7.65 (m, 1H), 7.18 (d, 2H), 6.99 (d, 2H), 4.88-4.81 (m, 1H), 4.08-4.06 (m, 1H), 3.92 (s, 3H), 3.45-3.40 (m, 1H), 3.34-3.27 (m, 1H), 3.11-3.01 (m, 5H), 2.97 (s, 3H), 1.82 (m, 1H), 1.66-1.57 (m, 2H), 1.45 (m, 1H).

¹³C NMR (CDCl₃): δ=173.1, 172.9, 159.1, 158.6, 150.4, 138.8, 133.4, 133.2, 130.3, 121.9, 117.3, 62.0, 53.1, 49.7, 39.4, 36.6, 36.5, 36.4, 30.4, 23.9.

### EXAMPLE 64

### Synthesis of N-(Toluene-4-sulfonyl)-L-(1,1-dioxo)thiaprolyl-L-4-(N,N-dimethylcarbamyloxy)phenylalanine

The title compound was prepared from the product of Example 61 using the procedure described in Method 11.

NMR data was as follows:

¹H NMR (CDCl₃): δ =8.34 (d, 1H), 7.70 (d, 2H), 7.33 (d, 2H), 7.14 (d, 2H), 7.01 (d, 2H), 5.07 (m, 1H), 4.93 (m, 1H), 4.43 (d, 1H), 4.01 (d, 1H), 3.68 (m, 1H), 3.37 (m, 1H), 3.17 (s, 3H), 3.14 (m, 1H), 3.09 (s, 3H), 2.54 (m, 1H), 2.43 (s, 3H).

¹³C NMR (CDCl₃): δ =171.5, 166.4, 156.4, 150.5, 145.5, 134.2, 134.1, 131.4, 130.3, 128.1, 121.8, 64.3, 59.2, 53.7, 50.5, 36.9, 36.5, 35.8, 21.6.

### EXAMPLE 65

### Synthesis of N-(4-Fluorobenzenesulfonyl)-L-thiaprolyl-L-4-(N,N-dimethylcarbamyloxy)phenylalanine

The title compound was prepared from the product of Example 84 using the procedure described in Method 11.

NMR data was as follows:

¹H NMR (CDCl₃): δ =7.83 (m, 2H), 7.73 (d, 1H), 7.16 (m, 4H), 6.99 (d, 2H), 5.57 (br s, 1H), 4.87 (m, 1H), 4.76 (m, 1H), 4.53 (d, 1H), 4.10 (d, 1H), 3.34 (m, 1H), 3.22 (d, 2H), 3.12 (s, 3H), 3.04 (s, 3H), 2.43 (m, 1H).

¹³C NMR (CDCl₃): δ =172.1, 168.7, 155.7, 150.5, 133.6, 133.1, 130.8, 130.7, 121.7, 116.9, 116.6, 65.3, 53.3, 51.3, 36.8, 36.4, 36.1, 33.4.

### EXAMPLE 66

### Synthesis of N-(4-Fluorobenzenesulfonyl)-L-(5,5-dimethyl)-thiaprolyl-L-4-(N,N-dimethylcarbamyloxy)phenylalanine tert-Butyl Ester

The title compound was prepared following the procedure described for the preparation of Example 2 with the substitution of appropriate starting materials.

NMR data was as follows:

¹H NMR (CDCl₃): δ =7.91 (m, 2H), 7.26 (m, 4H), 7.02 (d, 2H), 6.96 (d, 1H), 4.75 (m, 1H), 4.55 (d, 1H), 4.42 (d, 1H), 3.86 (s, 1H), 3.08 (s, 3H), 3.05 (m, 2H), 3.00 (s, 3H), 1.43 (s, 9H), 1.17 (s, 3H), 1.16 (s, 3H).

¹³C NMR (CDCl₃): δ =169.9, 168.1, 167.6, 164.2, 154.9, 150.6, 133.1, 132.2, 131.0, 130.9, 130.4, 121.7, 116.9, 116.6, 82.7, 73.5, 54.7, 53.7, 50.5, 37.8, 36.6, 36.4, 29.1, 27.8, 23.8.

### EXAMPLE 67

### Synthesis of N-(4-Fluorobenzenesulfonyl)-L-(1,1-dioxothiamorpholin-3-carbonyl)-L-4-(1,1-dioxothiomorpholin-4-ylcarbonyloxy)phenylalanine tert-Butyl Ester

The title compound was prepared following the procedure described for the preparation of Example 68.

NMR data was as follows:

¹H NMR (CDCl₃): δ =7.91-7.87 (m, 2H), 7.27-7.25 (m, 2H), 7.15 (d, 2H), 6.51 (d, 1H), 4.93-4.90 (m, 1H), 4.64-4.58 (m, 1H), 4.14-3.99 (m, 7H), 3.28-2.90 (m, 10H), 1.47 (s, 9H).

¹³C NMR (CDCl₃): δ =170.1, 167.6, 164.5, 153.1, 149.8, 133.9, 133.4, 130.7, 130.5, 121.7, 117.4, 117.1, 83.1, 56.1, 53.4, 51.6, 49.9, 48.9, 43.1, 41.6, 36.2, 27.8.

### EXAMPLE 68

### Synthesis of N-(Toluene-4-sulfonyl)-L-(1,1-dioxothiamorpholin-3-carbonyl)-L-4-(1,1-dioxothiomorpholin-4-ylcarbonyloxy)phenylalanine tert-Butyl Ester

Substituting thiomorpholine for N-methylpiperazine, and following the method for the preparation of Example 4 and oxidation of the sulfur group in the thiomorpholino ring per by Larsson and Carlson (Acta Chemica Scan. 1994, 48, 522) gave the title compound as a white solid.

NMR data was as follows:

¹H NMR (CDCl₃): δ =7.75 (d, 2H), 7.35 (d, 2H), 7.17 (d, 2H), 6.99 (d, 2H), 6.65 (d, 1H), 4.92-4.90 (m, 1H), 4.63-4.60 (m, 1H), 4.15-3.95 (m, 7H), 3.30-3.23 (m, 1H), 3.14 (t, 4H), 3.07-2.80 (m, 6H), 2.45 (s, 3H), 1.48 (s, 9H).

¹³C NMR (CDCl₃): δ =169.9, 164.8, 153.1, 149.8, 145.5, 135.1, 133.6, 130.7, 127.5, 121.8, 82.9, 60.3, 56.1, 53.7, 51.8, 49.3, 48.4, 43.1, 42.7, 41.5, 36.3, 27.8, 21.5.

### EXAMPLE 69

### Synthesis of N-(4-Fluorobenzenesulfonyl)-L-prolyl-L-4-(thiomorpholin-4-ylcarbonyloxy)phenylalanine tert-Butyl Ester

The title compound was prepared following the procedure described in Example 37 and substitution of appropriate starting materials.

NMR data was as follows:

¹H NMR (CDCl₃): δ =7.88-7.83 (m, 2H), 7.26-7.15 (m, 5H), 7.01 (d, 2H), 4.74-4.67 (m, 1H), 4.08-4.05 (m, 1H), 3.91-3.80 (m, 4H), 3.41-3.35 (m, 1H), 3.24-3.00 (m, 3H), 2.70-2.65 (t, 4H), 2.06-2.04 (m, 1H), 1.60-1.46 (m, 12H).

¹³C NMR (CDCl₃): δ =170.5, 169.8, 153.4, 150.2, 133.5, 130.7, 130.5, 130.3, 121.6, 116.8, 116.5, 82.6, 62.2, 53.6, 49.6, 47.0, 46.4, 37.2, 29.8, 27.8, 27.3, 27.0, 24.1.

### EXAMPLE 70

### Synthesis of N-(4-Fluorobenzenesulfonyl)-L-(5,5-dimethyl)-thiaprolyl-L-4-(N,N-dimethylcarbamyloxy)phenylalanine

The title compound was prepared from the product of Example 66 using the procedure described in Method 11.

NMR data was as follows:

¹H NMR (CDCl₃): δ =7.90 (m, 2H), 7.30-7.14 (m, 5H), 7.02 (d, 2H), 5.83 (br s, 1H), 4.90 (m, 1H), 4.57 (d, 1H), 4.40 (d, 1H), 3.96 (s, 1H), 3.09 (s, 3H), 3.28-3.02 (m, 2H), 3.00 (s, 3H), 1.13 (s, 6H).

¹³C NMR (CDCl₃): δ =173.2, 169.2, 164.2, 163.9, 155.3, 150.6, 133.1, 132.0, 131.0, 130.9, 130.6, 122.0, 117.0, 116.7, 73.3, 54.6, 53.3, 50.5, 37.0, 36.7, 36.4, 29.0, 23.7.

### EXAMPLE 71

### Synthesis of N-(4-Fluorobenzenesulfonyl)-L-(1,1-dioxothiamorpholin-3-carbonyl)-L-4-(morpholin-4-ylcarbonyloxy)phenylalanine tert-Butyl Ester

Substituting 4-morpholinecarbamyl chloride for dimethylcarbamyl chloride, and following the methods for the preparation of Example 2, gave the title compound as a white solid.

NMR data was as follows:

¹H NMR (CDCl₃): δ =7.91-7.87 (m, 2H), 7.26-7.20 (m, 2H), 7.11 (d, 2H), 6.98 (d, 2H), 6.43 (d, 1H), 4.95-4.92 (m, 1H), 4.62-4.60 (m, 1H), 4.05-4.00 (m, 2H), 3.74 (t, 4H), 3.66-3.52 (m, 4H), 3.30-2.92 (m, 6H), 1.48 (s, 9H).

¹³C NMR (CDCl₃): δ =170.1, 164.5, 150.4, 134.6, 132.7, 130.5, 122.0, 1 17.4, 117.1, 83.1, 66.5, 56.1, 53.4, 49.9, 49.0, 44.7, 44.0, 41.6, 36.2, 27.8.

### EXAMPLE 72

### Synthesis of N-(4-Trifluoromethoxybenzenesulfonyl)-L-prolyl-L-4-(N,N-dimethylcarbamyloxy)phenylalanine tert-Butyl Ester

The title compound was prepared following the procedure described for the preparation of Example 2 and substitution of appropriate starting materials.

NMR data was as follows:

¹H NMR (CDCl₃): δ =7.89 (d, 2H), 7.35 (d, 2H), 7.25-7.13 (m, 3H), 7.01 (d, 2H), 4.70 (m, 1H), 4.09-4.06 (m, 1H), 3.39-3.36 (m, 1H), 3.24-3.01 (m, 5H), 2.98 (s, 3H), 2.05 (m, 1H), 1.62-1.47 (m, 3H), 1.46 (s, 9H).

¹³C NMR (CDCl₃): δ =170.4, 169.9, 154.9, 152.7, 150.6, 134.6, 113.2, 130.2, 130.1, 121.7, 120.2, 82.7, 62.2, 53.6, 49.6, 37.2, 36.6, 36.3, 29.9, 27.8, 24.1.

### EXAMPLE 73

### Synthesis of N-(Toluene-4-sulfonyl)-L-(1,1-dioxothiamorpholin-3-carbonyl)-L-4-(N,N-dimethylcarbamyloxy)phenylalanine Isopropyl Ester

Following the method for the preparation of Example 2 and oxidation of the sulfur group in the thiomorpholino ring per by Larsson and Carlson (Acta Chemica Scan. 1994, 48, 522) gave the title compound.

NMR data was as follows:

¹H NMR (CDCl₃): δ =7.70 (d, 2H), 7.31 (d, 2H), 7.04 (d, 2H), 6.93 (d, 2H), 6.59 (d, 1H), 5.01 (m, 2H), 4.65 (m, 1H), 4.01 (d, 1H), 3.90 (d, 1H), 3.25 (m, 1H), 3.00 (s, 3H), 2.82 (m, 8H), 2.37 (s, 3H), 1.22 (s, 3H), 1.20 (s, 3H).

¹³C NMR (CDCl₃): δ =170.3, 165.0, 154.6, 150.5, 145.1, 135.2, 132.3, 130.4, 130.0, 127.2, 122.1, 69.5, 55.9, 53.1, 49.1, 48.5, 41.4, 36.3, 36.1, 35.9, 21.4.

### EXAMPLE 74

### Synthesis of N-(4-Fluorobenzenesulfonyl)-L-(1,1-dioxo-5,5-dimethyl)thiaprolyl-L-4-(N,N-dimethylcarbamyloxy)phenylalanine tert-Butyl Ester

The title compound was prepared from the product of Example 66 following the procedure described by Larsson and Carlson (Acta Chemica Scan. 1994, 48, 522).

NMR data was as follows:

¹H NMR (CDCl₃): δ =7.88 (m, 2H), 7.24 (m, 4H), 7.05 (d, 2H), 6.95 (d, 1H), 4.80 (m, 1H), 4.40 (m, 2H), 4.10 (s, 1H), 3.17-3.03 (m, 2H), 3.10 (s, 3H), 3.01 (s, 3H), 1.47 (s, 9H), 1.36 (s, 3H), 1.11 (s, 3H).

¹³C NMR (CDCl₃): δ =169.8, 168.6, 166.0, 154.5, 150.8, 139.7, 133.0, 131.5, 131.4, 130.3, 122.0, 117.1, 116.8, 83.0, 68.0, 60.9, 59.3, 53.8, 37.4, 36.6, 36.4, 27.8, 18.9, 18.8.

### EXAMPLE 75

### Synthesis of N-(Toluene-4-sulfonyl)-L-(1,1-dioxo-5,5-dimethyl)thiaprolyl-L-4-(N,N-dimethylcarbamyloxy)phenylalanine Isopropyl Ester

The title compound was prepared from the product of Example 11 following the procedure described by Larsson and Carlson (Acta Chemica Scan. 1994, 48, 522).

NMR data was as follows:

¹H NMR (CDCl₃): δ =7.75 (d, 2H), 7.38 (d, 2H), 7.21 (d, 2H), 7.03 (m, 3H), 5.08 (m, 1H), 4.89 (m, 1H), 4.38 (m, 2H), 4.10 (s, 1H), 3.22-3.04 (m, 2H), 3.10 (s, 3H), 3.00 (s, 3H), 2.43 (s, 3H), 1.26 (m, 9H), 1.09 (s, 3H).

¹³C NMR (CDCl₃): δ =170.3, 166.3, 150.8, 145.9, 132.8, 131.9, 130.3, 128.6, 122.0, 69.8, 68.0, 60.9, 59.4, 53.4, 37.4, 36.6, 36.4, 21.6, 21.5, 19.2, 18.6.

### EXAMPLE 76

### Synthesis of N-(4-Fluorobenzenesulfonyl)-L-(5,5-dimethyl)thiaprolyl-L-4-(N,N-dimethylcarbamyloxy)phenylalanine

The title compound was prepared from the product of Example 74 using the procedure described in Method 11.

NMR data was as follows:

¹³C NMR (CDCl₃): δ =171.7, 167.9, 137.3, 164.5, 155.9, 150.4, 133.6, 131.8, 131.3, 131.2, 130.8, 121.9, 117.1, 116.8, 67.8, 60.9, 59.9, 53.8, 36.8, 36.6, 36.0, 19.1, 19.0.

### EXAMPLE 77

### Synthesis of N-(Pyrimidine-2-sulfonyl)-L-prolyl-L-4-(N,N-dimethylcarbamyloxy)phenylalanine

The title compound was prepared from the product of Preparative Example B using the procedure described in Method 11.

NMR data was as follows:

¹H NMR (CDCl₃): δ =8.45 (br m, 2H), 8.22 (br s, 1H), 7.55 (d, 1H), 7.11 (d, 2H), 6.95 (d, 2H), 6.81 (m, 1H), 4.80-4.74 (m, 2H), 3.70 (m, 1H), 3.55 (m, 1H), 3.20-3.08 (m, 4H), 2.98 (s, 3H), 2.89-2.76 (m, 1H), 2.13-1.96 (m, 3H), 1.60 (m, 1H).

¹³C NMR (CDCl₃): δ =190.0, 173.6, 171.0, 155.2, 153.9, 150.6, 133.2, 130.1, 121.9, 110.3, 62.0, 55.1, 48.2, 36.6, 36.6, 36.3, 30.2, 23.4.

### EXAMPLE 78

### Synthesis of N-(Toluene-4-sulfonyl)-L-(1,1-dioxothiamorpholin-3-carbonyl)-L-4-(4-methylpiperazin-1-ylcarbonyloxy)phenylalanine tert-Butyl Ester

Following the method for the preparation of Example 4 and oxidation of the sulfur group in the thiamorpholino ring per Larsson and Carlson (Acta Chemica Scan. 1994, 48, 522) gave the title compound.

NMR data was as follows:

¹H NMR (CDCl₃): δ =7.76 (d, 2H), 7.37 (d, 2H), 7.12 (d, 2H), 6.96 (d, 2H), 6.57 (d, 1H), 4.95 (m, 1H), 4.62 (m, 1H), 4.03 (m, 2H), 3.67 (m, 4H), 3.25 (m, 1H), 2.89 (m, 4H), 2.45 (m, 6H), 2.35 (s, 3H), 1.48 (s, 9H).

¹³C NMR (CDCl₃): δ =170.0, 164.8, 153.7, 150.5, 145.4, 135.3, 132.8, 130.7, 130.4, 127.5, 122.2, 82.9, 56.2, 54.6, 54.5, 53.6, 49.5, 48.6, 46.0, 44.2, 43.7, 41.6, 36.3, 27.9, 21.6.

### EXAMPLE 79

### Synthesis of N-(4-Fluorobenzenesulfonyl)-L-(1,1-dioxo)thiaprolyl-L-4-(N,N-dimethylcarbamyloxy)phenylalanine

The title compound was prepared from the product of Example 85 using the procedure described in Method 11.

NMR data was as follows:

¹H NMR (CDCl₃): δ =4.98, (m, 1H), 4.90 (m, 1H), 4.44 (d, 1H), 4.03 (d, 1H), 3.67 (m, 1H), 3.37 (m, 1H), 3.25-3.02 (m, 1H), 3.20 (s, 3H), 3.11 (s, 3H), 2.68 (m, 1H).

¹³C NMR (CDCl₃): δ =171.7, 167.9, 166.3, 164.4, 157.0, 156.4, 150.5, 139.6, 134.0, 133.1, 131.3, 131.1, 130.9, 121.9, 117.2, 116.9, 64.1, 58.8, 53.7, 50.6, 36.9, 36.5, 35.6.

### EXAMPLE 80

### Synthesis of N-(Toluene-4-sulfonyl)-L-prolyl-L-4-(isonipecotoyloxy)phenylalanine tert-Butyl Ester

Substituting piperazine for N-methylpiperazine, and following the methods for the preparation of Example 4, gave the title compound as a white solid.

NMR data was as follows:

¹³C NMR (CDCl₃): δ =7.70 (d, 2H), 7.32-7.26 (m, 2H), 7.14 (d, 2H), 7.01 (d, 2H), 4.72-4.68 (m, 1H), 4.07-4.05 (m, 1H), 3.60-3.49 (m, 4H), 3.37-3.31 (m, 1H), 3.22-2.98 (m, 3H), 2.42 (s, 3H), 2.02 (m, 2H), 1.61-1.55 (m, 6H), 1.50-1.45 (m, 13H).

¹³C NMR (CDCl₃): δ =177.3, 170.7, 169.8, 150.6, 144.3, 133.1, 130.1, 129.9, 127.9, 121.6, 110.8, 82.5, 62.2, 57.2, 53.7, 49.5, 44.9, 37.2, 29.7, 27.8, 25.7, 24.1, 21.4.

### EXAMPLE 81

### Synthesis of N-(Toluene-4-sulfonyl)-L-prolyl-L-4-(1,1-dioxothiomorpholin-4-ylcarbonyloxy)phenylalanine tert-Butyl Ester

The product of Example 82 was oxidized by the method of Larsson and Carlson (Acta Chemica Scan. 1994, 48, 517-525), yielding the title compound as a white solid.

NMR data was as follows:

¹H NMR (CDCl₃): δ =7.69 (d, 2H), 7.33-7.29 (m, 3H), 7.20 (d, 2H), 7.00 (d, 2H), 4.71-4.66 (m, 1H), 4.13-4.04 (m, 5H), 3.37-3.32 (m, 1H), 3.21-3.00 (m, 7H), 2.41 (s, 3H), 2.05-2.01 (m, 1H), 1.52-1.44 (m, 12H).

¹³C NMR (CDCl₃): δ =170.7, 169.7, 149.8, 144.3, 134.4, 133.3, 130.6, 130.0, 127.9, 121.4, 82.7, 62.4, 54.0, 52.1, 49.7, 43.2, 37.6, 29.7, 28.1, 24.4, 21.7.

### EXAMPLE 82

### Synthesis of N-(Toluene-4-sulfonyl)-L-prolyl-L-4-(thiomorpholin-4-ylcarbonyloxy)phenylalanine tert-Butyl Ester

Substituting thiomorpholine for N-methylpiperazine, and following the methods for the preparation of Example 4, gave the title compound as a white solid.

NMR data was as follows:

¹³C NMR (CDCl₃): δ =7.70 (d, 2H), 7.31-7.26 (m, 2H), 7.16 (d, 2H), 7.00 (d, 2H), 4.72-4.66 (m, 1H), 4.07-4.04 (m, 1H), 3.89-3.79 (m, 4H), 3.37-3.32 (m, 1H), 3.22-2.99 (m, 3H), 2.67 (t, 4H), 2.42 (s, 3H), 2.02 (m, 2H), 1.50-1.45 (m, 12H).

¹³C NMR (CDCl₃): δ =177.2, 170.7, 169.8, 153.5, 150.2, 144.3, 133.6, 132.9, 130.3, 129.9, 127.9, 121.5, 82.5, 62.4, 53.7, 49.5, 47.0, 46.4, 37.2, 29.6, 27.8, 27.3, 24.1, 21.4.

### EXAMPLE 83

### Synthesis of N-(Toluene-4-sulfonyl)-L-prolyl-L-4-(pyrrolidin-1-ylcarbonyloxy)phenylalanine tert-Butyl Ester

Substituting pyrrolidinecarbonyl chloride for dimethylcarbamyl chloride, and following the methods for the preparation of Example 2, gave the title compound as a white solid.

NMR data was as follows:

¹H NMR (CDCl₃): δ =7.71 (d, 2H), 7.32 (d, 2H), 7.15 (d, 2H), 7.04 (d, 2H), 4.73-4.67 (m, 1H), 4.07-4.04 (m, 1H), 3.53 (t, 2H), 3.45 (t, 2H), 3.36-3.32 (m, 1H), 3.24-2.98 (m, 3H), 2.42 (s, 3H), 2.03-1.88 (m, 5H), 1.75 (s, 1H), 1.52 (1.24 (m, 12H).

¹³C NMR (CDCl₃): δ =170.7, 169.8, 153.1, 150.4, 144.3, 133.1, 130.1, 129.9, 127.9, 121.6, 110.8, 99.8, 82.5, 62.2, 53.7, 49.5, 46.3, 37.2, 29.7, 27.8, 25.6, 24.8, 24.0, 21.4.

### EXAMPLE 84

### Synthesis of N-(4-Fluorobenzenesulfonyl)-L-thiaprolyl-L-4-(N,N-dimethylcarbamyloxy)phenylalanine tert-Butyl Ester

The title compound was prepared following the procedures described for the preparation of Example 2 and substitution of appropriate starting materials.

NMR data was as follows:

¹H NMR (CDCl₃): δ =7.87 (m, 2H), 7.28-7.13 (m, 5H), 7.02 (d, 2H), 4.70-4.60 (m, 2H), 4.58 (d, 1H), 4.06 (d, 1H), 3.38-3.01 (m, 3H), 3.09 (s, 3H), 3.00 (s, 3H), 2.58 (m, 1H), 1.47 (s, 9H).

¹³C NMR (CDCl₃): δ =169.7, 167.8, 154.9, 150.7, 132.7, 130.9, 130.7, 130.4, 121.8, 117.1, 116.8, 82.9, 65.1, 53.9, 51.4, 36.8, 36.6, 36.4, 33.1, 27.9.

### EXAMPLE 85

### Synthesis of N-(4-Fluorobenzenesulfonyl)-L-(1,1-dioxo)thiaprolyl-L-4-(N,N-dimethylcarbamyloxy)phenylalanine tert-Butyl Ester

The title compound was prepared from the product of Example 84 following the procedure oxidation procedure of Larsson and Carlson (Acta Chemica Scan. 1994, 48, 517-525).

NMR data was as follows:

¹H NMR (CDCl₃): δ =7.90 (m, 2H), 7.30-7.04 (m, 7H), 4.83-4.58 (m, 3H), 3.66 (m, 2H), 3.32-3.24 (m, 1H), 3.09-2.85 (m, 2H), 3.10 (s, 3H), 3.01 (s, 3H), 1.50 (s, 9H).

¹³C NMR (CDCl₃): δ =173.1, 169.8, 168.0, 165.6, 154.9, 150.9, 132.6, 131.1, 131.0, 130.3, 122.3, 117.3, 117.0, 83.2, 62.8, 57.8, 53.9, 49.0, 36.8, 36.6, 36.4, 27.9.

### EXAMPLE 86

### Synthesis of N-(2,5-Dichlorothiophene-3-sulfonyl)-L-prolyl-L-4-(N,N-dimethylcarbamyloxy)phenylalanine tert-Butyl Ester

The title compound was prepared following the procedure described for the preparation of Example 2 and substitution of appropriate starting materials.

NMR data was as follows:

¹H NMR (CDCl₃): δ =7.14 (d, 2H), 7.09 (s, 1H), 7.07 (d, 1H), 7.01 (d, 2H), 4.73-4.66 (m, 1H), 4.32-4.28 (m, 1H), 3.42-3.17 (m, 3H), 3.08 (s, 3H), 3.06-3.01 (m, 1H), 2.98 (s, 3H), 2.17-2.04 (m, 1H), 1.84-1.60 (m, 2H), 1.60-1.46 (m, 1H), 1.45 (s, 9H).

¹³C NMR (CDCl₃): δ =170.2, 169.9, 154.9, 150.6, 133.4, 133.1, 131.2, 130.2, 127.9, 127.0, 121.7, 82.7, 62.2, 53.6, 49.3, 37.2, 36.6, 36.4, 30.1, 27.8, 24.2.

### EXAMPLE 87

### Synthesis of N-(4-Acetamidobenzenesulfonyl)-L-prolyl-L-4-(N,N-dimethylcarbamyloxy)phenylalanine tert-Butyl Ester

The title compound was prepared following the procedure described for the preparation of Example 2 and substitution of appropriate starting materials.

NMR data was as follows:

¹H NMR (CDCl₃): δ =8.58 (s, 1H), 7.70-7.67 (m, 4H), 7.32 (d, 1H), 7.14 (d, 2H), 7.01 (d, 2H), 4.68 (m, 1H), 3.99 (m, 1H), 3.37-3.34 (m, 1H), 3.23-3.16 (m, 1H), 3.11-3.01 (m, 1H), 3.08 (s, 3H), 2.98 (s, 3H), 2.13 (s, 3H), 1.97-1.94 (m, 1H), 1.55-1.47 (m, 3H), 1.44 (s, 9H).

¹³C NMR (CDCl₃): δ =171.1, 169.9, 169.4, 155.0, 150.6, 143.3, 133.3, 130.2, 130.0, 128.9, 121.7, 119.4, 82.7, 62.2, 53.8, 49.6, 37.2, 36.6, 36.4, 29.9, 27.8, 24.4, 24.1.

### EXAMPLE 88

### Synthesis of N-(4-tert-Butylbenzenesulfonyl)-L-(1,1-dioxothiamorpholin-3-carbonyl)-L-4-(N,N-dimethylcarbamyloxy)phenylalanine tert-Butyl Ester

The title compound was prepared following the procedure outlined for the preparation of Example 73 and substitution of appropriate starting materials.

NMR data was as follows:

¹H NMR (CDCl₃): δ =7.81 (d, 2H), 7.59 (d, 2H), 7.07 (d, 2H), 6.97 (d, 2H), 6.46 (d, 1H), 4.95 (m, 1H), 4.62 (m, 1H), 4.06 (m, 2H), 3.23 (m, 1H), 3.07 (m, 4H), 2.97 (m, 4H), 2.81 (m, 4H), 1.55 (s, 9H), 1.37 (s, 9H).

¹³C NMR (CDCl₃): δ =170.0, 164.9, 158.2, 154.8, 150.6, 135.0, 132.6, 130.2, 127.4, 126.9, 122.2, 82.7, 56.1, 53.5, 49.7, 48.8, 41.5, 36.5, 36.3, 36.1, 35.2, 30.8, 27.8.

### EXAMPLE 89

### Synthesis of N-(Pyridine-3-sulfonyl)-L-prolyl-L-4-(N,N-dimethylcarbamyloxy)phenylalanine

The title compound was prepared from the product of Example 56 using the procedure described in Method 11.

NMR data was as follows:

¹H NMR (CD₃OD): δ=8.95 (s, 1H), 8.83 (d, 1H), 8.28-8.24 (m, 1H), 7.73-7.69 (m, 1H), 7.30 (d, 2H), 7.05 (d, 2H), 4.68-4.63 (m, 1H), 4.29-4.25 (m, 1H), 3.47-3.41 (m, 1H), 3.38-3.22 (m, 2H), 3.09 (s, 3H), 3.06-3.02 (m, 1H), 2.96 (s, 3H), 1.92-1.66 (m, 4H).

¹³C NMR (CD₃OD): δ=174.2, 173.9, 160.6, 160.0, 156.9, 152.9, 152.0, 147.9, 139.1, 136.9, 135.7, 131.6, 126.5, 123.1, 63.1, 54.8, 50.4, 37.5, 36.8, 36.7, 32.2, 25.5.

### EXAMPLE 90

### Synthesis of N-(2-Fluorobenzenesulfonyl)-L-(1,1-dioxothiamorpholin-3-carbonyl)-L-4-(N,N-dimethylcarbamloxy)phenylalanine tert-Butyl Ester

L-Thiamorpholine-3-carboxylic acid was prepared by the method of Larsson and Carlson (Acta Chemica Scan. 1994, 48, 517-525). N-(2-fluorobenzene-4-sulfonyl)-L-thiamorpholine-3-carboxylic acid was prepared using the procedure described in Method 1. The title compound was prepared according to the procedures set forth above using suitable starting materials.

NMR data was as follows:

¹H NMR (CDCl₃): δ =7.92 (m, 1H), 7.69 (m, 1H), 7.34 (m, 2H), 7.16 (m, 2H), 6.99 (m, 2H), 6.60 (d, 1H), 5.01 (m, 1H), 4.64 (m, 1H), 4.03 (m, 2H), 3.29 (m, 1H), 3.06 (m, 6H), 2.90 (m, 7H), 1.49 (d, 9H).

¹³C NMR (CDCl₃): δ =169.9, 164.8, 160.3, 156.9, 154.9, 150.7, 136.6, 136.4, 132.7, 131.0, 130.3, 128.8, 126.4, 126.2, 125.1, 122.2, 118.1, 117.8, 82.7, 56.3, 56.7, 50.2, 49.5, 41.8, 36.5, 36.3, 27.8.

### EXAMPLE 91

### Synthesis of N-(3-Fluorobenzenesulfonyl)-L-(1,1-dioxothiamorpholin-3-carbonyl)-L-4-(N,N-dimethylcarbamyloxy)phenylalanine tert-Butyl Ester

L-Thiamorpholine-5-carboxylic acid was prepared by the method of Larsson and Carlson (Acta Chemica Scan. 1994, 48, 517-525). N-(3-fluorobenzene-4-sulfonyl)-L-thiamorpholine-5-carboxylic acid was prepared using the procedure described in Method 1. The title compound was prepared according to the procedures set forth above using suitable starting materials.

NMR data was as follows:

¹H NMR (CDCl₃): δ =7.66 (m, 1H), 7.58 (m, 2H), 7.34 (m, 1H), 7.07 (d, 1H), 6.92 (d, 1H), 6.42 (d, 1H), 5.00 (m, 1H), 4.58 (m, 1H), 4.02 (m, 2H), 3.22 (m, 1H), 3.05 (s, 3H), 2.98 (m, 6H), 1.45 (s, 9H).

¹³C NMR (CDCl₃): δ =170.0, 164.5, 164.4, 161.0, 154.9, 150.6, 140.3, 140.2, 132.5, 131.9, 131.8, 130.2, 123.2, 123.1, 122.2, 121.4, 121.2, 115.0, 114.7, 82.9, 56.1, 53.4, 49.9, 49.1, 41.7, 36.5, 36.3, 36.0, 27.8.

### EXAMPLE 92

### Synthesis of N-(2,4-Difluorobenzenesulfonyl)-L-(1,1-dioxothiamorpholin-3-carbonyl)-L-4-(N,N-dimethylcarbamyloxy)phenylalanine tert-Butyl Ester

L-Thiamorpholine-5-carboxylic acid was prepared by the method of Larsson and Carlson (Acta Chemica Scan. 1994, 48, 517-525). N-(2,4-difluorobenzene-4-sulfonyl)-L-thiamorpholine-5-carboxylic acid was prepared using the procedure described in Method 1. The title compound was prepared according to the procedures set forth above using suitable starting materials.

NMR data was as follows:

¹H NMR (CDCl₃): δ =7.93 (m, 1H), 7.15 (m, 2H), 7.04 (m, 4H), 6.53 (d, 1H), 4.97 (m, 1H), 4.64 (m, 1H), 4.05 (m, 2H), 3.21 (m, 3H), 3.17 (s, 3H), 2.97 (m, 5H), 1.43 (s, 9H).

¹³C NMR (CDCl₃): δ =170.0, 164.6, 154.9, 150.7, 132.6, 132.6, 130.3, 122.6, 122.1, 112.6, 112.3, 107.0, 106.7, 106.3, 82.8, 56.3, 53.5, 50.5, 49.8, 42.0, 36.5, 36.3, 27.8.

### EXAMPLE 93

### Synthesis of N-(4-Acetamidobenzenesulfonyl)-L-prolyl-L-4-(N,N-dimethylcarbamyloxy)phenylalanine

The title compound was prepared from the product of Example 87 using the procedure described in Method 11.

NMR data was as follows:

¹H NMR (CD₃OD): δ=8.05 (d, 1H), 7.78 (m, 4H), 7.26 (d, 2H), 7.02 (d, 2H), 4.94 (m, 1H), 4.72-4.67 (m, 1H), 4.13-4.09 (m, 1H), 3.40-3.36 (m, 1H), 3.30-3.05 (m, 3H), 3.08 (s, 3H), 2.97 (s, 3H), 2.15 (s, 3H), 1.81-1.51 (m, 4H).

¹³C NMR (CD₃OD): δ=174.3, 174.2, 172.3, 156.9, 152.0, 144.9, 135.5, 132.4, 131.6, 130.2, 122.9, 120.7, 63.2, 54.7, 50.6, 37.5, 36.8, 36.7, 31.7, 25.4, 24.0.

### EXAMPLE 94

### Synthesis of N-(4-Trifluoromethoxybenzenesulfonyl)-L-prolyl-L-4-(N,N-dimethylcarbamyloxy)phenylalanine

The title compound was prepared from the product of Example 72 using the procedure described in Method 11.

NMR data was as follows:

¹H NMR (CD₃OD): δ=7.93 (d, 2H), 7.48 (d, 2H), 7.28 (d, 2H), 7.03 (d, 2H), 4.72-4.68 (m, 1H), 4.17-4.13 (m, 1H), 3.45-3.42 (m, 1H), 3.28-3.11 (m, 2H), 3.14-3.07 (m, 1H), 3.09 (s, 3H), 2.97 (s, 3H), 1.85-1.69 (m, 3H), 1.59 (m, 1H).

¹³C NMR (CD₃OD): δ=174.2, 174.1, 157.0, 153.9, 152.0, 137.3, 135.6, 131.7, 131.5, 123.0, 122.5, 121.8, 63.1, 54.7, 50.6, 37.4, 36.8, 36.6, 31.9, 25.4.

### EXAMPLE 95

### Synthesis of N-(4-Fluorobenzenesulfonyl)-L-(5,5-dimethyl)thiaprolyl-L-4-(N,N-dimethylcarbamyloxy)phenylalanine tert-Butyl Ester

The title compound was prepared using the procedure described in Example 2 and substitution of appropriate starting materials.

NMR data was as follows:

¹H NMR (CDCl₃): δ =7.90 (m, 2H), 7.22 (m, 4H), 7.00 (m, 3H), 5.08 (m, 1H), 4.84 (m, 1H), 4.56 (d, 1H), 4.42 (d, 1H), 3.88 (s, 1H), 3.15-2.99 (m, 2H), 3.09 (s, 3H), 3.00 (s, 3H), 1.26-1.16 (m, 12H).

¹³C NMR (CDCl₃): δ =170.4, 168.2, 167.5, 164.1, 154.9, 150.7, 132.8, 132.2, 132.1, 131.0, 130.8, 130.3, 121.8, 116.9, 116.6, 73.5, 69.6, 54.6, 53.2, 50.5, 37.6, 36.6, 36.3, 29.1, 23.8, 21.6, 21.5.

### EXAMPLE 96

### Synthesis of N-(4—Cyanobenzenesulfonyl)-L-prolyl-L-4-(N,N-dimethylcarbamyloxy)phenylalanine

The title compound was prepared from the product of Example 58 using the procedure described in Method 11.

NMR data was as follows:

¹H NMR (CD₃OD): δ=8.14 (d, 1H), 7.94-7.89 (m, 4H), 7.29 (d, 2H), 7.03 (d, 2H), 4.70-4.66 (m, 1H), 4.21-4.17 (m, 1H), 3.47-3.40 (m, 1H), 3.31-3.21 (m, 2H), 3.11-3.04 (m, 1H), 3.09 (s, 3H), 2.97 (s, 3H), 1.87-1.72 (m, 3H), 1.70-1.61 (m, 1H).

¹³C NMR (CD₃OD): δ=174.2, 173.9, 157.0, 152.0, 142.9, 135.7, 134.5, 131.7, 129.7, 123.0, 118.6, 111.8, 63.0, 54.7, 50.5, 37.4, 36.8, 36.7, 32.0, 25.4.

### EXAMPLE 97

### Synthesis of N-(Toluene-4-sulfonyl)-L-(3,3-dimethyl)prolyl-L-4-(N,N-dimethylcarbamyloxy)phenylalanine tert-Butyl Ester

The title compound was prepared using the procedure described for the preparation of Example 98.

NMR data was as follows:

¹H NMR (CDCl₃): δ =7.76 (d, 1H), 7.75 (d, 1H), 7.35 (d, 1H), 7.34 (d, 1H), 7.33 (d, 1H), 7.20 (d, 1H), 7.10 (d, 1H), 7.03 (d, 1H), 6.91 (d, 0.5H), 6.08 (d, 0.5H), 4.86 (ddd, 0.5H), 4.77 (q, 0.5H), 3.61-3.47 (m, 2H), 3.27-3.02 (m, 3H), 3.09 (s, 3H), 3.00 (s, 3H), 2.45 (s, 1.5H), 2.43 (s, 1.5H), 1.75-1.68 (m, 0.5H), 1.61-1.51 (m, 0.5H), 1.45 (s, 4.5H), 1.40 (s, 4.5H), 1.48-1.25 (m, 3H); 0.95 (s, 1.5H), 0.80 (s, 1.5H); 0.61 (s, 1.5H).

¹³C NMR (CDCl₃): δ =170.4, 170.1, 170.0, 169.6, 155.0, 154.9, 150.7, 150.6, 144.3, 144.2, 133.4, 133.1, 132.8, 132.6, 130.7, 130.2, 129.9, 129.8, 128.0, 121.8, 121.7, 82.6, 82.2, 71.5, 71.2, 53.6, 52.7, 47.3, 47.2, 42.7, 42.5, 38.2, 38.1, 37.7, 37.5, 36.6, 36.3, 27.8, 27.8, 27.2, 23.4, 23.2, 21.5.

### EXAMPLE 98

### Synthesis of N-(Toluene-4-sulfonyl)-L-(3,3-dimethyl)prolyl-L-4-(N,N-dimethylcarbamyloxy)phenylalanine Isopropyl Ester

3,3-Dimethyl proline (see Sharma and Lubell, J. Org. Chem. 1996, 61, 202-209) was N-tosylated using the procedure described in Method 1. The title compound was then prepared following the procedure described for the preparation of Example 2.

NMR data was as follows:

¹H NMR (CDCl₃): δ =7.76 (d, 1H), 7.74 (d, 1H), 7.36 (d, 1H), 7.33 (d, 2H), 7.19 (d, 1H), 7.10 (d, 1H), 7.03 (d, 1H), 6.91 (d, 0.5H), 6.89 (d, 0.5H), 5.06 (sept., 0.5H), 4.96 (sept., 0.5H), 4.98-4.83 (m, 1H), 3.59-3.48 (m, 2H), 3.31-3.03 (m, 3H), 3.09 (s, 3H), 3.00 (s, 3H), 2.45 (s, 1.5H), 2.43 (s, 1.5H), 1.75-1.66 (m, 0.5), 1.62-1.52 (m, 0.5H), 1.34-1.22 (m, 3H), 1.27 (s, 1.5H), 1.25 (s, 1.5H), 1.22 (s, 1.5H), 1.20 (s, 1.5H), 0.95 (s, 1.5H), 0.78 (s, 1.5H), 0.60 (s, 1.5H), 0.57 (s, 1.5H).

¹³C NMR (CDCl₃): δ =170.8, 170.6, 170.0, 169.7, 154.9, 150.8, 150.6, 144.4, 144.2, 133.2, 132.5, 132.5, 130.7, 130.2, 129.9, 129.8, 128.0, 122.0, 121.8, 71.5, 17.2, 69.5, 69.3, 53.0, 52.2, 47.3, 47.2, 42.8, 42.5, 38.2, 38.1, 37.6, 37.2, 36.6, 36.3, 27.1, 23.4, 23.2, 21.6, 21.6, 21.5, 21.5.

Other compounds prepared by the methods described above include those set forth in Examples 99-137. In addition, Examples 101, 109, 111, 117, 132 and 137 are exemplified as follows:

### EXAMPLE 101

### Synthesis of N-(Toluene-4-sulfonyl)-L-prolyl-4-(N,N-dimethylcarbamyloxy)-L-phenylalanine Isopropyl Ester

The title compound was prepared following the procedure outlined for the preparation of Example 2.

NMR data was as follows:

¹H NMR (CD₃)₂SO: δ=8.28 (d, 1H), 7.70 (d, 2H), 7.41 (d, 2H), 7.23 (d, 2H), 6.99 (d, 2H), 4.86 (sept, 1H), 4.47 (m, 1H), 4.40 (m, 1H), 4.10 (m, 1H), 4.07 (m, 1H), 3.38 (m, 1H), 3.30 (m, 1H), 3.09 (m, 3H), 2.95 (s, 3H), 3.00 (s, 3H), 2.88 (s, 3H), 2.39 (s, 3H), 1.63 (m, 3H), 1.51 (m, 3H), 1.44 (m, 1H), 1.39 (m, 1H), 1.16 (d, 3H), 1.11 (d, 3H).

¹³C NMR (CD₃)₂SO: δ=171.3, 170.8, 154.2, 150.2, 143.7, 134.1, 130.2, 130, 127.6, 121.6, 68.2, 61.2, 53.5, 49, 36.3, 36.1, 35.7, 30.5, 23.8, 21.4, 21.4, 21.

### EXAMPLE 109

### Synthesis of N-(Benzylsulfonyl)-L-(5,5-dimethyl) thiaprolyl-L-(4-N,N-dimethylcarbamyloxy)phenylalanine

The title compound was prepared from the product of Example 111 using the procedure described in Method 11.

Physical data was as follows:

MS (FAB) (M+H)+550.

Calcd. for: C₂₅H₃₁N₃O₇S₂; C, 54.62; H, 5.68; N 7.64. Found: C 54.51; H 5.60; N 7.63.

### EXAMPLE 111

### Synthesis of N-(Benzylsulfonyl)-L-(5,5-dimethyl) thiaprolyl-L-(4-N,N-dimethylcarbamyloxy)phenylalanine tert-Butyl Ester

The title compound was prepared following the procedure outlined for the preparation of Example 2 and by substituting the appropriate starting materials.

Physical data was as follows:

MS [M+H]⁺ 550.

Calcd. for: C₂₉H₃₉N₃O₇S₂; C, 57.52; H, 6.45; N, 6.94. Found: C, 57.32; H, 6.52; N, 6.81.

### EXAMPLE 117

### Synthesis of N-(Methyl-pyrazole-4-sulfonyl)-L-prolyl-L-(4-N,N-dimethylcarbamyloxy)phenylalanine tert-Butyl Ester

Substituting N-methyl-pyrazole sulfonyl chloride (see Dickson, U.S. Pat. No. 3,665,009 (May 23, 1972) and following the method for the preparation of Example 56, gave the title compound.

NMR data was as follows:

¹H NMR (CDCl₃): δ =7.83 (s, 1H), 7.76 (s, 1H), 7.26 (m, 1H), 7.15 (m, 2H), 7.00 (m, 2H), 4.69 (m, 1H), 3.95 (m, 1H), 3.93 (s, 3H), 3.38 (m, 1H), 3.23-3.11 (m, 1H), 3.10-2.99 (m, 4H), 2.99 (s, 3H), 2.05 (m, 1H), 1.66-1.46 (m, 3H), 1.44 (s, 9H).

¹³C NMR (CDCl₃): δ 170.7, 169.9, 154.9, 150.6, 138.9, 133.2, 132.5, 130.2, 121.7, 117.9, 82.6, 62.4, 53.7, 49.7, 39.6, 37.7, 36.6, 36.4, 29.9, 27.9, 24.2.

### EXAMPLE 132

### Synthesis of N-(4-Fluorobenzenesulfonyl)-L-prolyl-L-4-(1,1-dioxothiomorpholin-4-ylcarbonyloxy)-phenylalanine tert-Butyl Ester

Substituting thiamorpholine for N-methylpiperazine, and following the method for the preparation of Example 4 and 14, gave the title compound.

NMR data was as follows:

¹H NMR (CDCl₃): δ =7.87-7.82 (m, 2H), 7.28-7.17 (m, 5H), 7.01 (d, 2H), 4.71-4.69 (m, 1H), 4.14-4.05 (m, 5H), 3.39-3.36 (m, 1H), 3.23-3.01 (m, 7H), 2.05-2.03 (m, 1H), 1.58-1.44 (m, 12H).

¹³C NMR (CDCl₃): δ =170.4, 169.8, 153.0, 149.7, 134.2, 130.6, 130.5, 121.3, 116.8, 116.5, 82.6, 62.1, 53.6, 51.8, 49.5, 43.1, 42.7, 37.2, 29.7, 27.8, 24.2.

### EXAMPLE 137

### Synthesis of N-(Methyl-pyrazole-4-sulfonyl)-L-prolyl-L-(4-N,N-dimethylcarbamyloxy)-phenylalanine Isopropyl Ester

The title compound was prepared following the procedure described for the preparation of Example 117.

NMR data was as follows:

¹H NMR (CDCl₃): δ 7.83 (s, 1H), 7.76 (s, 1H), 7.27 (d, 1H), 7.13 (d, 2H), 7.01 (d, 2H), 5.06-5.02 (m, 1H), 4.80-4.73 (m, 1H), 3.97-3.94 (m, 1H), 3.93 (s, 3H), 3.44-3.37 (m, 1H), 3.25-3.19 (m, 1H), 3.09-3.00 (m, 5H), 2.97 (s, 3H), 2.06-2.02 (m, 1H), 1.66-1.48 (m, 3H), 1.23 (d, 6H).

¹³C NMR (CDCl₃): δ 170.8, 170.5, 154.9, 150.6, 138.9, 132.9, 32.5, 130.2, 121.7, 117.8, 69.5, 62.3, 53.2, 49.7, 39.6, 37.1, 36.6, 36.3, 29.9, 24.1, 21.6, 21.5.

TABLE 12

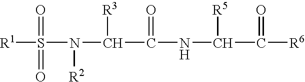

| | | | | | Ex. |
|---|---|---|---|---|---|
| R¹ | R² | R³ | R⁵ | R⁶ | No. |
| p-CH₃-φ- | R²/R³ = cyclic | | p-[(4-methylpiperazin-1-yl)C(O)O-]benzyl- | —O-n-butyl | 99 |
| | 3 carbon atoms | | | | |
| | (L-pyrrolidinyl) | | | | |
| p-CH₃-φ- | R²/R³ = cyclic | | p-[(4-methylpiperazin-1-yl)C(O)O-]benzyl- | —O-cyclopentyl | 100 |
| | 3 carbon atoms | | | | |
| | (L-pyrrolidinyl) | | | | |
| p-CH₃-φ- | R²/R³ = cyclic | | p-[(CH₃)₂NC(O)O-]benzyl- | —OCH(CH₃)₂ | 101 |
| | 3 carbon atoms | | | | |
| | (L-pyrrolidinyl) | | | | |
| p-CH₃-φ- | R²/R³ = cyclic | | p-[(piperidin-4-yl)C(O)O-]benzyl- | —OCH₂CH₃ | 102 |
| | 3 carbon atoms | | | | |
| | (L-pyrrolidinyl) | | | | |
| φ-CH₂— | R²/R³ = cyclic | | p-[(1-methylpiperidin-4-yl)C(O)O-]benzyl- | —OCH₂CH₃ | 103 |
| | 3 carbon atoms | | | | |
| | (L-pyrrolidinyl) | | | | |
| φ-CH₂- | R²/R³ = cyclic | | p-[(4-methylpiperazin-1-yl)C(O)O-]benzyl- | —OH | 104 |
| | 3 carbon atoms | | | | |
| | (L-pyrrolidinyl) | | | | |
| p-CH₃-φ- | R²/R³ = cyclic | | p-[(1-Boc-4-phenylpiperidin-4-yl)- | —OCH₂CH₃ | 105 |
| | 3 carbon atoms | | C(O)O-]benzyl- | | |
| | (L-pyrrolidinyl) | | | | |
| 1- | R²/R³ = cyclic | | p-[(CH₃)₂NC(O)O-]benzyl- | —OC(CH₃)₃ | 106 |
| methylimidazol-4-yl | 3 carbon atoms | | | | |
| | (L-pyrrolidinyl) | | | | |
| p-NH₂-φ- | R²/R³ = cyclic | | p-[(CH₃)₂NC(O)O-]benzyl- | —OC(CH₃)₃ | 107 |
| | 3 carbon atoms | | | | |
| | (L-pyrrolidinyl) | | | | |
| p-CH₃-φ- | R²/R³ = cyclic | | p-[(morpholin-4-yl)C(O)O-]benzyl- | —OC(CH₃)₃ | 108 |
| | 3 carbon atoms | | | | |
| | (L-pyrrolidinyl) | | | | |
| φ-CH₂— | R²/R³ = cyclic | | p-[(CH₃)₂NC(O)O-]benzyl- | —OH | 109 |
| | —CH₂—S—C(CH₃)₂— | | | | |
| | (L-5,5- | | | | |
| | dimethylthiazolidin- | | | | |
| | 4-yl) | | | | |
| p-CH₃-φ- | R²/R³ = cyclic | | p-[(CH₃)₂NC(O)O-]benzyl- | —OH | 110 |
| | —CH₂CH₂—NH—CH₂— | | | | |
| | (L-piperazinyl) | | | | |
| φ-CH₂— | R²/R³ = cyclic | | p-[(CH₃)₂NC(O)O-]benzyl- | —OC(CH₃)₃ | 111 |
| | —CH₂—S—C(CH₃)₂— | | | | |
| | (L-5,5- | | | | |
| | dimethylthiazolidin | | | | |
| | 4-yl) | | | | |
| p-CH₃-φ- | R²/R³ = cyclic | | p-[(CH₃)₂NC(O)O-]benzyl- | —NH-adamantyl | 112 |
| | 3 carbon atoms | | | | |
| | (L-pyrrolidinyl) | | | | |
| p-CH₃-φ- | R²/R³ = cyclic | | p-[(CH₃)₂NC(O)O-]benzyl- | —NHCH₂C(O)OH | 113 |
| | 3 carbon atoms | | | | |
| | (L-pyrrolidinyl) | | | | |
| p-CH₃-φ- | R²/R³ = cyclic | | p-[(CH₃)₂NS(O)₂O-]benzyl- | —OCH₃ | 114 |
| | 3 carbon atoms | | | | |
| | (L-pyrrolidinyl) | | | | |
| p-CH₃-φ- | R²/R³ = cyclic | | p-[(CH₃)₂NC(O)O-]benzyl- | —OC(CH₃)₃ | 115 |
| | —CH₂CH₂—NH—CH₂— | | | | |
| | (L-piperazinyl) | | | | |
| p-CH₃-φ- | R²/R³ = cyclic | | p-[(CH₃)₂NC(O)O-]benzyl- | —OC(CH₃)₃ | 116 |
| | —CH₂CH₂— | | | | |
| | (Cbz)NHCH₂- | | | | |
| | [L-4-N-(Cbz)- | | | | |
| | piperazinyl] | | | | |
| 1- | R²/R³ = cyclic | | p-[(CH₃)₂NC(O)O-]benzyl- | —OC(CH₃)₃ | 117 |
| methylpyrazol- | 3 carbon atoms | | | | |
| 4-yl | (L-pyrrolidinyl) | | | | |
| 3-pyridyl | R²/R³ = cyclic | | p-[(CH₃)₂NC(O)O-]benzyl- | —OH | 118 |
| | 3 carbon atoms | | | | |
| | (L-pyrrolidinyl) | | | | |
| p-CH₃-φ | R²/R³ = cyclic | | p-[(1-Boc-piperazin-4-yl)C(O)O-]benzyl- | —OCH₂CH₃ | 119 |
| | 3 carbon atoms | | | | |
| | (L-pyrrolidinyl) | | | | |
| p-CH₃-φ | R²/R³ = cyclic | | p-[(morpholin-4-yl)C(O)O-]benzyl- | —OCH₂CH₃ | 120 |
| | 3 carbon atoms | | | | |
| | (L-pyrrolidinyl) | | | | |
| p-CH₃-φ | —CH₃ | H | p-[(thiomorpholin-4-yl sulfone)- | —OC(CH₃)₃ | 121 |
| | | | C(O)O-]benzyl- | | |
| p-CH₃-φ | —CH₃ | H | p-[(thiomorpholin-4-yl)C(O)O-]benzyl- | —OH | 122 |
| p-CH₃-φ | R²/R³ = cyclic | | p-[(CH₃)₂NC(O)O-]benzyl- | 2,4-dioxo- | 123 |
| | 3 carbon atoms | | | tetrahydrofuran- | |
| | (L-pyrrolidinyl) | | | 3-yl(3,4-enol) | |
| p-CH₃-φ | R²/R³ = cyclic | | p-[(piperazin-4-yl)C(O)O-]benzyl- | —OH | 124 |
| | 3 carbon atoms | | | | |
| | (L-pyrrolidinyl) | | | | |
| p-CH₃-φ | R²/R³ = cyclic | | p-[(1-Boc-piperazin-4-yl)C(O)O-]benzyl- | —OC(CH₃)₃ | 125 |
| | 3 carbon atoms | | | | |
| | (L-pyrrolidinyl) | | | | |
| p-CH₃-φ | R²/R³ = cyclic | | p-[(piperazin-4-yl)C(O)O-]benzyl- | —OCH₂CH₃ | 126 |
| | 3 carbon atoms | | | | |
| | (L-pyrrolidinyl) | | | | |
| p-CH₃-φ | R²/R³ = cyclic | | p-[(4-acetylpiperazin-1-yl)C(O)O-]benzyl- | —OCH₂CH₃ | 127 |
| | 3 carbon atoms | | | | |
| | (L-pyrrolidinyl) | | | | |
| p-CH₃-φ | R²/R³ = cyclic | | p-[(4-methanesulfonylpiperazin-1-yl)- | —OCH₂CH₃ | 128 |
| | 3 carbon atoms | | C(O)O-]benzyl- | | |
| | (L-pyrrolidinyl) | | | | |
| p-CH₃-φ | R²/R³ = cyclic | | 3-nitro-4-[(morpholin-4-yl)- | —OH | 129 |
| | 3 carbon atoms | | C(O)O-]benzyl | | |
| | (L-pyrrolidinyl) | | | | |
| p-CH₃-φ | R²/R³ = cyclic | | p-[(1-Boc-piperazin-4-yl)C(O)O-]benzyl- | —OH | 130 |
| | 3 carbon atoms | | | | |
| | (L-pyrrolidinyl) | | | | |
| p-CH₃-φ | —CH₃ | —C(CH₃)₃ | p-[(4-methylpiperazin-1-yl)C(O)O-]benzyl- | —OC(CH₃)₃ | 131 |
| p-F-φ | R²/R³ = cyclic | | p-[(1,1-dioxothiomorpholin-4-yl)- | —OC(CH₃)₃ | 132 |
| | 3 carbon atoms | | C(O)O-]benzyl | | |
| | (L-pyrrolidinyl) | | | | |
| p-F-φ | R²/R³ = cyclic | | p-[(morpholin-4-yl)C(O)O-]benzyl- | —OC(CH₃)₃ | 133 |
| | 3 carbon atoms | | | | |
| | (L-pyrrolidinyl) | | | | |
| p-CH₃-φ | R²/R³ = cyclic | | p-[(morpholin-4-yl)C(O)O-]benzyl- | —OH | 134 |
| | —CH₂—CH₂—SO₂—CH₂— | | | | |
| | (L-1,1-dioxothiomorpholin | | | | |
| | 3-yl | | | | |
| 1- | R²/R³ = cyclic | | p-[(4-methylpiperazin-1-yl)C(O)O-]benzyl- | —OC(CH₃)₃ | 135 |
| methylpyrazol-4-yl | 3 carbon atoms | | | | |
| | (L-pyrrolidinyl) | | | | |
| morpholin- | R²/R³ = cyclic | | p-[(CH₃)₂NC(O)O-]benzyl- | —OC(CH₃)₃ | 136 |
| 4-yl | 3 carbon atoms | | | | |
| 1- | R²/R³ = cyclic | | p-[(CH₃)₂NC(O)O-]benzyl- | —OCH(CH₃)₂ | 137 |
| methylpyrazol- | 3 carbon atoms | | | | |
| 4-yl | (L-pyrrolidinyl) | | | | |

Additional compounds prepared by the methods described above include the following:

### EXAMPLE 138

### Synthesis of N-(1-Methylpyrazole-4-sulfonyl)-L-(5,5-dimethyl)thiaprolyl-L-4-(N,N-dimethylcarbamyloxy)phenylalanine tert-Butyl Ester

The N-methylpyrazole sulfonyl chloride was prepared by adding N-methylpyrazole to chilled (0° C.) chlorosulfonic acid. The reaction mixture was allowed to warm to room temperature and then heated to 100° C. overnight under a stream of N₂. The reaction mixture was then cooled to room temperature and chilled to 0° C. To this solution was added thionyl chloride (2.5 eq.) and the reaction was stirred at room temperature for 30 min., then warmed to 70° C. for two hours. The reaction was cooled to room temperature and then chilled in an ice bath. Water and ice were slowly added to the reaction mixture to precipitate a white solid which was collected by filtration. The desired sulfonyl chloride was washed with cold water and hexane.

The title compound was then prepared following the procedure outlined for the preparation of Example 2 by substitution of the appropriate starting materials, mp: 169-170° C.

### EXAMPLE 139

### Synthesis of N-(1-Methylpyrazole-4-sulfonyl)-L-(5,5-dimethyl)thiaprolyl-L-4-(N,N-dimethylcarbamyloxy)phenylalanine

The title compound was prepared from the product of Example 138 using the procedure described in Method 11.

NMR data was as follows:

¹H NMR (CDCl₃): δ=7.94 (s, 1H); 7.79 (s, 1H); 7.25 (d, 2H, J=8.8 Hz); 7.0 (d, 2H, J=8.8 Hz); 5.15 (br s, 1H); 4.80 (m, 1H); 4.54 (d, 1H, J=9.HHz); 4.39 (d, 1H, J=9.3 Hz); 3.93 (s, 3H); 3.88 (s, 1H); 3.23-3.02 (m, 2H0; 3.07 (s, 3H); 2.98 (s, 3H); 1.27 (s, 3H); 1.14 (s, 3H).

¹³C NMR (CDCl₃): 173.86, 169.05, 155.23, 150.47, 139.21, 133.59, 133.15, 130.53, 121.84, 117.57, 73, 58, 54, 71, 53.75, 50.42, 39.60, 37.18, 36.60, 36.36, 35.11, 28.97, 23.95.

### EXAMPLE 140

### Synthesis of N-(Toluene-4-sulfonyl)-L-prolyl-L-4-(N-(1,4-dioxa-8-aza-spiro[4.5]decan-8-yl)carbonyloxy)phenylalanine Ethyl Ester

The title compound was prepared following the procedure outlined for Example 4 by substitution of the appropriate starting materials.

Physical data was as follows:

MS (+ESI): 630 [M+H]+

Anal. Calcd. for C₃₁H₃₉N₃O₉S.0.2 CH₂Cl₂: C, 57.94; H, 6.14; N, 6.50. Found: C, 57.73; H, 5.90; N, 6.47.

### EXAMPLE 141

### Synthesis of N-(Toluene-4-sulfonyl)-L-prolyl-L-4-(N-(1,4-dioxa-8-aza-spiro[4.5]decan-8-yl)carbonyloxy)phenylalanine

The product of Example 140 was hydrolyzed using the procedure described in Method 5 but employing methanol as the solvent and running the reaction at 25° C. for 24 h. The solvent was then evaporated, the residue taken up in H₂O, washed with methylene chloride and lyophilized to afford the title compound.

Physical data was as follows:

MS (+ESI): 619 [M+H]⁺.

Anal. Calcd. for C₂₉H₃₅N₃O₉SLi.1.5H₂O: C, 53.37; H, 6.02; N, 6.44. Found: C, 53.40; H, 5.58; N, 6.48.

### EXAMPLE 142

### Synthesis of N-(Toluene-4-sulfonyl)-L-prolyl-L-4-(4′-acetylpiperazin-1-ylcarbonyloxy)phenylalanine

The product of Example 127 was hydrolyzed using the procedure described in Method 5 but employing methanol as the solvent and running the reaction at 25° C. for 24 h. The solvent was then evaporated, the residue taken up in H₂O, washed with methylene chloride and lyophilized to afford the title compound.

Physical data was as follows:

MS (+ESI): 587 [M+H]⁺.

Anal. Calcd. for C₂₈H₃₃N₄O₈SLi.3H₂O: C, 52.01; H, 6.08; N, 8.66. Found: C, 52.03; H, 5.36; N, 8.04.

### EXAMPLE 143

### Synthesis of N-(Toluene-4-sulfonyl)-L-prolyl-L-4-(4′-methanesulfonylpiperazin-1-ylcarbonyloxy)phenylalanine

The product of Example 128 was hydrolyzed using the procedure described in Example 142.

Physical data was as follows:

MS (+ESI): 623 [M+H]⁺.

Anal. Calcd. for C₂₇H₃₃N₄O₉S₂Li ˜2H₂O: C, 48.79; H, 5.61; N, 8.43. Found: C, 48.66; H, 5.14; N, 8.04.

### EXAMPLE 144

### Synthesis of N-(Toluene-4-sulfonyl)-L-prolyl-L-4-(4′-phenylpiperazin-1-ylcarbonyloxy)phenylalanine

The ethyl ester of the title compound was prepared following the procedure outlined for Example 4 by substitution of the appropriate starting materials. The ethyl ester was then hydrolyzed using the procedure described in Example 142.

Physical data was as follows:

MS (−ESI): 619 [M−H]⁻.

Anal. Calcd. for C₃₂H₃₆N₄O₇SLi.2H₂O: C, 58.00; H, 5.93; N, 8.45. Found: C, 57.65; H, 5.49; N, 8.13.

### EXAMPLE 145

### Synthesis of N-(Toluene-4-sulfonyl)-L-prolyl-L-4-(piperazin-ly-ylcarbonyloxy)phenylalanine tert-Butyl Ester

The product of Example 125 (0.7 g, 1 mmol) was dissolved in methylene chloride (9 mL). The solution was cooled to 0° C. and trifluoroacetic acid (1.0 mL) was added and the resulting clear solution was stirred for 4 h. The reaction solution was then diluted with additional methylene chloride (50 m]L), washed with saturated sodium bicarbonate solution (3×50 mL), dried (K₂CO₃) and the solvent stripped off to give a white solid (0.465 g). Flash chromatography (9:1 CH₂Cl₂:EtOH) of this material gave a clear oil which was washed several times with hexane to give a white solid (0.289 g, 48%).

Physical data was as follows:

MS (+ESI): 601.7 [M+1]⁺.

Anal. Calcd. for C₃₀H₄₀N₄O₇S.0.25 CH₂Cl₂: C, 58.42; H, 6.56; N, 9.01. Found: C, 58.79; H, 6.51; N, 8.74.

### EXAMPLE 146

### Synthesis of 2-(Saccharin-2-yl)propionyl-L-4-(4′-methylpiperazin-1-ylcarbonyloxy)phenylalanine,

The title compound was prepared from the product of Example 46 using the procedure described in Method 11, mp=117-122° C. (with foaming).

Physical data was as follows:

Anal. Calcd. for C₂₅H₂₈N₄O₈S.1.5H₂O: C, 52.53; H, 5.47; N, 9.80. Found: C, 52.26; H, 5.36; N, 9.23.

### EXAMPLE 147

### Synthesis of N-(Toluene-4-sulfonyl)-L-prolyl-L-4-(4′-methanesulfonyl piperazin-1-ylcarbonyloxy)phenylalanine tert-Butyl Ester

The title compound was prepared following the procedure outlined for Example 128 by substitution of the appropriate starting materials.

Physical data was as follows:

MS (+ESI): 696 [M+NH₄]⁺.

Anal. Calcd. for C₃₁H₄₂N₄O₉S₂.0.5 CH₂Cl₂: C, 51.62; H, 6.00; N, 7.76. Found: C, 51.55; H, 6.21; N, 7.60.

### EXAMPLE 148

### Synthesis of N-(Toluene-4-sulfonyl)-L-prolyl-L-4-(N,N-dimethylcarbamyloxy)phenylalanine (N-tert-butoxycarbonyl-2-amino-2-methylpropyl) Ester

(BOC)₂₀ (96 mg, 0.44 mmol) was added to a solution of the product from Example 9 (200 mg, 0.4 mmol.), N-Boc-2-amino-2-methyl-1-propanol (965 mg, 0.5 mmol) and a catalytic amount of DMAP in THF (92 mL) containing pyridine (50 μl). The mixture was stirred at room temperature under argon for 48 h. The mixture was poured into 1N HCl and extracted with ethyl acetate. The organic phase was washed (1N HCl), dried (MgSO₄) and the solvent was removed under reduced pressure. The residue was purified by flash chromatography (EtOAc:hexanes 2:1) to give the desired compound as an amorphous white foam (150 mg., 55%).

Physical data was as follows:

MS: [M+H]+at 675.

MS (+ESI): [M+NH₄]+at 692 (100%).

### EXAMPLE 149

### Synthesis of N-(Toluene-4-sulfonyl)-L-prolyl-L-4-(N,N-dimethylcarbamyloxy)phenylalanine 2-(Morpholin-4-yl)ethyl Ester

The title compound was prepared following the procedure outlined for Example 148 by substituting 2-morpholinoethanol for N-Boc-2-amino-2-methyl-1-propanol.

Physical data was as follows:

Anal. Calcd. for C₃₀H₄₀N₄O₈S.0.5H₂O: C, 57.58; H, 6.60; N, 8.95. Found: C, 57.26; H, 6.29; N, 8.82.

### EXAMPLE 150

### Synthesis of N-(Toluene-4-sulfonyl)-L-prolyl-L-4-(4′-acetylpiperazin-1-ylcarbonyloxy)phenylalanine tert-Butyl Ester

The title compound was prepared following the procedure outlined for Example 127 by substitution of the appropriate starting materials.

Physical data was as follows:

MS (+ESI): 660.4 [M+NH₄]⁺.

Anal. Calcd. for C₃₂N₄₂N₄O₈S 0.15 CH₂Cl₂: C, 58.91; H, 6.50; N, 8.55. Found: C, 58.64; H, 6.36; N, 8.40.

### EXAMPLE 151

### Synthesis of N-(Toluene-4-sulfonyl)-L-prolyl-L-4-(4-hydroxypiperidin-1-ylcarbonyloxy)phenylalanine tert-Butyl Ester

The title compound was prepared following the procedure outlined for Example 4 by substituting 4-piperidinol for N-methyl piperazine.

Physical data was as follows:

Anal. Calcd. for C₃₁H₄₁N₃O₈S.0.6H₂O.0.22 EtOAc: C, 59.28; H, 6.86; N, 6.51 Found: C, 58.92; H, 6.37; N, 6.47.

### EXAMPLE 152

### Synthesis of N-(Toluene-4-sulfonyl)-L-prolyl-L-4-(N-(2-(morpholin-4′-yl)ethyl)carbamyloxy)phenylalanine tert-Butyl Ester

The title compound was prepared following the procedure outlined for Example 4 by substituting 4-(2-aminoethyl)morpholine for N-methyl piperazine.

Physical data was as follows:

Anal. Calcd. for C₃₂H₄₄N₄O₈S.0.25H₂O: C, 59.20; H, 6.91; N, 8.63 Found: C, 59.01; H, 6.54; N, 8.38.

### EXAMPLE 153

### Synthesis of N-(Toluene-4-sulfonyl)-L-prolyl-L-4-(N-(1,4-dioxa-8-aza-spiro[4.5]decan-8-yl)carbonyloxy)phenylalanine tert-Butyl Ester

The title compound was prepared following the procedure outlined for Example 4 by substitution of the appropriate starting materials.

Physical data was as follows:

MS (−ESI): 656 [M−H]⁻.

Anal. Calcd. for C₃₃H₄₃N₃O₉S.0.1 CH₂Cl₂: C, 59.67; H, 6.54; N, 6.31. Found: C, 59.83; H, 6.63; N, 6.66.

### EXAMPLE 154

### Synthesis of N-(Toluene-4-sulfonyl)-L-prolyl-L-4-(N-(2-hydroxyethyl)-N-methylcarbamyloxy)phenylalanine tert-Butyl Ester

The title compound was prepared folllowing the procedure outlined for Example 4 by substituting 2-(methylamino)ethanol for N-methyl piperazine.

Physical data was as follows:

Anal. Calcd. for C₂₉H₃₉N₃O₈S.0.5H₂O: C, 58.18; H, 6.73; N, 7.02. Found: C, 57.95; H, 6.5; N, 6.9.

### EXAMPLE 155

### Synthesis of N-(Toluene-4-sulfonyl)-L-prolyl-L-4-(4-formyloxypiperidin-1-ylcarbonyloxy)phenylalanine

The title compound was prepared by treating the product of Example 151 with formic acid overnight with stirring. The title compound was obtained as a white foam (130 mg., 94%), following removal of excess formic acid.

NMR data was as follows:

¹H NMR (DMSO-d₆, 400 MHZ) δ 12.8 (s, 1H); 8.23 (s, 1H); 8.09 (d, 1H); 7.69 (d, 2H), 7.4 (d, 2H); 7.23 (d, 2H), 7.02 (d, 2H); 5.00 (m, 1H); 4.45 (m, 1H); 4.10 (m, 1H); 3.6-3.8 (br, 2H); 3.4 (br s, 1H); 3.25 (m, 2H); 3.10 (m, 2H); 2.95 (m, 1H); 2.35 (s, 3H); 1.95 (m, 2H); 1.56-1.75 (m, 5H); 1.4 (m, 1H).

IR (KBr, cm⁻¹) 3400, 2950, 1720, 1680, 1510, 1430, 1325, 1250, 1150, 1010, 650, 75, 540.

MS ((+) ESI, m/z (%)) 605 (100 [M+NH, 1⁺).

Anal. Calcd. for C₂₈H₃₃N₃O₉S.0.66H₂O: C, 56.09; H, 5.77; N, 7.01. Found: C, 56.14; H, 5.83; N, 6.78.

### EXAMPLE 156

### Synthesis of N-(Toluene-4-sulfonyl)-L-prolyl-L-4-(4′-hydroxypiperidin-1-ylcarbonyloxy)phenylalanine Isopropyl Ester

The title compound was prepared following the procedure outlined for Example 4 by substitution of the appropriate starting materials, mp. 64-67° C. (with foaming).

Physical data was as follows:

Anal. Calcd. for C₃₀H₃₉N₃O₈S.0.75H₂O.0.1 EtOAc: C, 58.51; H, 6.67; N, 6.73. Found: C, 58.55; H, 6.09; N, 6.78.

### EXAMPLE 157

### Synthesis of N-(Toluene-4-sulfonyl)-L-prolyl-L-4-(4′-(2-hydroxyethyl)piperazin-1-ylcarbonyloxy)phenylalanine tert-Butyl Ester

The carbonate was prepared by treatment of Tos-Pro-Tyr-t-butyl ester with 4-nitrophenyl chloroformate, followed by addition of N-(2-hydroxyl ethyl)piperazine (triethylamine, methylene chloride, chilled to 0° C., then stirred at room temperature overnight). The crude product was purified by flash chromatography (silica, 95:5 EtOAc:EtOH) to afford a white solid, mp. 158-160° C. (0.387 g, 58%).

NMR data was as follows:

¹H NMR (DMSO-d₆, 400 MHZ) δ 8.15 (d, 1H, J=7.90 Hz); 7.70 (d, 2H, J=6.59 Hz); 7.40 (d, 2H, J=7.90 Hz); 7.23 (d, 2H, J=8.56 Hz); 7.00 (d, 2H, J=8.56 Hz); 4.42 (m, 1H); 4.38 (m, 1H); 4.08 (m, 1H); 3.51 (m, 4H); 3.34 (m, 3H); 3.09 (m, 1H); 2.99 (m, 2H); 2.43 (m, 6H); 2.39 (s, 3H); 1.59 (m, 3H); 1.39 (m, 1H); 1.35 (s, 9H).

IR (KBr, cm⁻¹) 3505, 3400, 2990, 2930, 2890, 1730, 1700, 1670, 1510, 1430, 1350, 1220, 1200, 1160, 670, 590, 545.

MS ((−) ESI, m/z (%)) 643 (98 [M-NH₄]).

Anal. Calcd. for C₃₂H₄₄N₄O₈S: C, 59.61; H, 6.88; N, 8.69. Found: C, 59.06; H, 6.95; N, 8.43.

### EXAMPLE 158

### Synthesis of N-(Toluene-4-sulfonyl)-L-prolyl-L-4-(N-(2-formyloxyethyl)-N-methylcarbamyloxy)phenylalanine

The title compound was prepared by treating the product of Example 154 with formic acid overnight with stirring. The title compound was obtained as a white foam (110 mg., 77%), following removal of excess formic acid.

NMR data was as follows:

¹H NMR (DMSO-d₆, 400 MHz) δ 12.8 (s, 1H); 8.25 (d, 1H); 8.08 (d, 1H); 7.69 (d, 2H), 7.40 (d, 2H); 7.22 (d, 2H), 6.98 (dd, 2H); 4.47 (m, 1H); 4.35 (m, 1H); 4.27 (m, 1H); 4.10 (m, 1H); 3.65 (m, 1H); 3.55 (m, 1H); 2.85-3.15 (overlapping m, 7H); 2.40 (s, 3H); 1.55 (m, 3H); 1.40 (m, 1H).

IR (KBr, cm⁻¹) 3420, 2910, 1725, 1510, 1400, 1340, 1270, 1150, 675, 590, 550.

MS ((+) ESI, m/z (%)) 579 (100 [M+NH, 1⁺).

Anal. Calcd. for C₂₆H₃₁N₃O₉S.0.66H₂O: C, 54.45; H, 5.68; N, 7.33 Found: C, 54.41; H, 5.60; N, 7.24.

### EXAMPLE 159

### Synthesis of N-(Toluene-4-sulfonyl)-L-prolyl-L-4-(N-(2′-hydroxyethyl)-N-methylcarbamyloxy)phenylalanine Isopropyl Ester

The title compound was prepared following the procedure outlined for Example 4 by substitution of the appropriate starting materials, mp. 49-52° C.

Physical data was as follows:

Anal Calcd. for C₂₈H₃₇N₃O₈S.0.5H₂O: C, 57.52; H, 6.55; N, 7.19. Found: C, 57.56; H, 6.38; N, 7.14.

### EXAMPLE 160

### Synthesis of N-(Toluene-4-sulfonyl)-L-prolyl-4-(N-(methoxycarbonylmethyl)carbamyloxy)phenylalanine tert-Butyl Ester

The carbonate was prepared by treatment of Tos-Pro-Tyr-t-butyl ester with 4-nitrophenyl chloroformate, followed by addition of glycine methyl ester (triethylamine, methylene chloride, chilled to 0°, then stirred at room temperature overnight). The crude product was purified by flash chromatography (silica, 3:2 EtOAc:hexane) to afford a white foam (0.640 g, 35%).

NMR data was as follows:

¹H NMR (DMSO-d₆, 400 MHz) δ 8.15 (d, 1H, J=8.12 Hz); 8.12 (d, 2H, J=6.15 Hz); 7.73 (d, 2H, J=8.34 Hz); 7.40 (d, 2H, J=7.90 Hz); 7.24 (d, 2H, J=8.56 Hz); 6.98 (d, 2H, J=8.34 Hz); 4.25 (m, 1H); 4.07 (m, 1H); 3.83 (d, 2H, J=6.15 Hz); 3.64 (s, 3H); 3.32 (m, 1H); 3.02 (m, 3H); 2.39 (s, 3H); 1.56 (m, 3H); 1.41 (m, 1H); 1.35 (s, 9H).

IR (KBr, CM⁻¹) 3400, 2990, 1745, 1680, 1500, 1370, 1350, 1200, 1160, 670, 600.

MS ((+) ESI, m/z (%)) 621 (100[M+NH₄]⁺).

Anal. Calcd. for C₂₉H₃₇N₃O₉S: c, 57.70; H, 6.18; N, 6.96. Found: C, 57.63; H, 6.11; N, 6.74.

### EXAMPLE 161

### Synthesis of N-(1-Methylpyrazole-4-sulfonyl)-L-(5,5-dimethyl) thiaprolyl-L-4-(N,N-dimethylcarbamyloxy)phenylalanine Isopropyl Ester

The title compound was prepared following the procedure outlined for Example 138 by substitution of the appropriate starting materials.

NMR data was as follows:

¹H NMR (CDCl₃): δ 7.91 (s, 1H), 7.81 (s, 1H), 7.21 (d, 2H, J=8.2 Hz), 7.03 (m, 3H); 5.03 (m, 1H), 4.84 (m, 1H), 4.55 (d, 1H), 4.42 (d, 1H), 3.96 (s, 3H), 3.83 (s, 1H), 3.18-3.01 (m, 2H), 3.10 (s, 3H), 3.01 (s, 3H), 1.28 (s, 3H), 1.24 (m, 6H), 1.17 (s, 3H).

¹³C NMR (CDCl₃): δ 170.43, 166.31, 154.92, 150.68, 132.91, 132.88, 130.34, 121.78, 117.69, 73.76, 69.61, 54, 79, 53.2, 50.52, 39.61, 37.62, 36.58, 36.35, 28.96, 24.02, 21.57, 21.49.

### EXAMPLE 162

### Synthesis of N-(Toluene-4-sulfonyl)-L-prolyl-L-4-(4′-methoxypiperidin-1-ylcarbonyloxy)phenylalanine Isopropyl Ester

The title compound was prepared following the procedure outlined for Example 156 by substitution of the appropriate starting materials.

NMR data was as follows:

¹H NMR (DSMO-d₆, 400 MHz) δ 8.10 (d, 1H); 7.72 (d, 2H); 7.41 (d, 2H); 7.24 (d, 2H); 7.02 (d, 2H); 4.92 (m, 1H); 4.45 (m, 1H); 4.10 (m, 1H); 3.8 (br s, 1H); 3.65 (br s, 1H); 3.40 (M, 2H); 3.25 (s, 3H); 2.95-3.15 (overlapping m, 5H); 2.40 (s, 3H); 1.85 (br, 2H); 1.4-1.6 (m, 6H); 1.18 (d, 3H); 1.12 (d, 3H).

IR (KBr, cm⁻¹) 3400, 2950, 1720, 1520, 1425, 1340, 1210, 1160, 1100, 625, 590, 540.

MS ((+) ESI, m/z (%)) 633 [M+NH]⁺).

### EXAMPLE 163

### Synthesis of N-(Toluene-4-sulfonyl)-L-prolyl-L-4-(4′-methoxypiperidin-1-ylcarbonyloxy)phenylalanine

The title compound was prepared from the product of Example 162 using the procedure described in Method 5.

NMR data was as follows:

¹H NMR (DMSO-d₆, 400 MHz) δ 12.8 (s, 1H); 8.10 (d, 1H); 7.72 (d, 2H); 7.41 (d, 2H); 7.24 (d, 2H); 7.02 (d, 2H); 4.45 (m, 1H); 4.10 (m, 1H); 3.8 (br s, 1H); 3.65 (br s, 1H); 3.40 (m, 2H); 3.25 (s, 3H); 2.95-3.15 (overlapping m, 5H); 2.40 (s, 3H); 1.85 (br, 2H); 1.4-1.6 (m, 6H).

IR (KBr, cm⁻¹) 3400, 2950, 1720, 1520, 1425, 1340, 1210, 1160, 1100, 625, 590, 540.

MS ((−) ESI, m/z (%)) 572 (100 [M−H]⁻).

Anal. Calcd. for C₂₈H₃₅N₃O₈S.0.33EtOAc.1H₂O: C, 56.73; H, 6.44; N, 6.77. Found: C, 56.96; H, 6.01; N, 6.76.

### EXAMPLE 164

### Synthesis of N-(Toluene-4-sulfonyl)-L-4-oxoprolyl-L-4-(N,N-dimethylcarbamyloxy)phenylalanine tert-Butyl Ester

Dichloromethane (7 mL) was cooled to −60° C. (chloroform/dry ice bath). Oxalyl chloride (0.15 mL) was added. The product from Example 165 (870 mg) and dry DMSO (0.26 mL) were dissolved in dichloromethane (8 mL) and added slowly to the above solution. The reaction was stirred at −60° C. for 30 minutes under dry conditions. Triethylamine (1.05 mL) was added. After 5 minutes, the dry ice bath was removed. The reaction was stirred at room temperature for 1 hour. The solvent was evaporated in vacuo. Ethyl acetate (30 mL) was added to the residue. The mixture was washed with citric acid solution (5%, 2×30 mL) and saturated NaHCO₃ solution (2×30 mL); and finally with brine. The solution was dried over MgSO₄. The solvent was evaporated in vacuo, and the residue was flushed on a silica gel column to give 440 mg of the desired product, mp: 78-80° C.

### EXAMPLE 165

### Synthesis of N-(Toluene-4-sulfonyl)-L-trans-4-hydroxyprolyl-L-4-(N,N-dimethylcarbamyloxy)phenylalanine tert-Butyl Ester

N-(Toluene-4-sulfonyl)-L-trans-4-hydroxyprolyl-L-4-(hydroxy)phenylalanine tert-butyl ester (1.60 g) and dimethylcarbamyl chloride (0.30 mL) were dissolved in DMF at 0° C. in an ice bath. Potassium carbonate powder (2.03 g) was added to the solution. The ice bath was removed after 5 minutes. The reaction was stirred at room temperature for 6 hours. The solid was filtered. Ethyl acetate (40 mL) was added to the solution. The solution was washed with citric acid solution (5%, 40 mL) 2 times, and saturated NaHCO₃ solution (40 mL) 1 time. The solution was then washed with brine and dried with MgSO₄. The solvent was evaporated in vacuo to give 1.07 g of the title compound, mp: 170-172° C.

### EXAMPLE 166

### Synthesis of N-(3-Fluorobenzenesulfonyl)-L-prolyl-L-4-(N,N-dimethylcarbamyloxy)phenylalanine tert-Butyl Ester

N-(3-Fluorobenzenesulfonyl)-L-prolyl-L-4-(hydroxy)phenylalanine tert-butyl ester (700 mg) and dimethylcarbamyl chloride (0.2 mL) were dissolved in DMF (15 mL) at 0° C. in an ice bath. Potassium carbonate powder (1.375 g) was added to the solution. The ice bath was removed after 5 minutes. The reaction was stirred at room temperature for 6 hours. The solid was filtered. Ethyl acetate (20 mL) was added to the solution. The solution was washed with citric acid solution (5%, 30 mL, 2×), and saturated NaHCO₃ solution. The solution was then washed with brine and dried with MgSO₄. The solvent was evaporated in vacuo to give 890 mg of the title compound, mp: 107-109° C.

### EXAMPLE 167

### Synthesis of N-(Morpholino-sulfonyl)-L-prolyl-L-(4-N,N-dimethylcarbamyloxy)phenylalanine tert-Butyl Ester

N-(Morpholino-sulfonyl)-L-proline was prepared using the procedure described by Cheeseright et al., J. Chem. Soc. Perkin Trans. 1 1994, 12, 1595-1600. The title compound was prepared following the procedure described for the preparation of Example 2.

NMR data was as follows:

¹H NMR (CDCl₃): δ 7.13 (d, 2H), 7.03 (d, 2H), 6.92 (d, 1H), 4.71 (q, 1H), 4.25 (t, 1H), 3.67 (t, 4H), 3.39 (dt, 1H), 3.28-3.19 (m, 1H), 3.23 (t, 4H), 3.18 (dd, 1H), 3.08 (dd, 1H), 3.09 (s, 3H), 3.00 (s, 3H), 2.16-2.08 (m, 2H), 1.98-1.86 (m, 1H), 1.78-1.66 (m, 1H), 1.45 (s, 9H).

¹³C NMR (CDCl₃): δ 171.2, 170.4, 154.8, 150.7, 132.9, 130.3, 121.7, 82.7, 66.3, 62.6, 53.3, 49.6, 46.2, 37.0, 36.6, 36.3, 30.5, 27.8, 24.7.

### EXAMPLE 168

### Synthesis of N-(Morpholino-sulfonyl)-L-prolyl-L-(4-N,N-dimethylcarbamyloxy)phenylalanine

The title compound was prepared from the product of Example 167 using the procedure described in Method 11.

NMR data was as follows:

¹H NMR (CD₃OD): δ 8.04 (d, 1H), 7.25 (d, 2H), 7.01 (d, 2H), 4.71-4.64 (m, 1H), 4.22 (dd, 1H), 3.62-3.50 (m, 4H), 3.43-3.31 (m, 2H), 3.24 (dd, 1H), 3.11 (t, 4H), 3.09 (s, 3H), 3.03 (dd, 1H), 2.97 (s, 3H), 2.22-2.11 (m, 1H), 1.98-1.80 (m, 3H).

¹³C NMR (CD₃OD): δ 174.65, 174.58, 174.00, 156.60, 151.70, 135.30, 131.20, 122.70, 67.10, 63.10, 54.59, 54.50, 50.6, 47.10, 37.10, 36.50, 36.40, 32.0, 25.60.

### EXAMPLE 169

### Synthesis of N-(1-Methylpyrazole-4-sulfonyl)-L-(1,1-dioxothiamorpholin-3-carbonyl)-L-4-(N,N-dimethylcarbamyloxy)phenylalanine tert-Butyl Ester

The title compound was prepared following the procedures described for the preparation of Examples 14 and 117.

NMR data was as follows:

¹H NMR (CDCl₃): δ 7.93 (s, 1H), 7.80 (s, 1H), 7.12 (d, 2H), 6.98 (d, 2H), 6.44 (d, 1H0HH), 4.95 (m, 1H), 4.66 (m, 1H), 4.04 (m, 2H), 3.98 (s, 3H), 3.19 (m, 2H), 3.06 (m, 6H), 2.98 (m, 4H), 1.42 (m, 9H).

¹³C NMR (CDCl₃): δ 170.58, 164.75, 154.91, 150.75, 139.33, 132.73, 132.43, 130.43, 122.18, 119.66, 83.07, 56.02, 53.23, 50.03, 49.03, 41.49, 39.63, 36.56, 36.31, 36.16, 27.87.

### EXAMPLE 170

### Synthesis of N-(2-Fluorobenzenesulfonyl)-L-(1,1-dioxothiamorpholin-3-carbonyl)-L-4-(N,N-dimethylcarbamyloxy)phenylalanine

The title compound was prepared from the product of Example 90 using the procedure described in Method 11.

NMR data was as follows:

¹H NMR (CD₃OD): δ 7.90 (m, 1H), 7.72 (m, 1H), 7.56 (d, 1H), 7.37 (m, 2H), 7.20 (d, 2H), 7.07 (d, 2H), 5.18 (m, 1H), 4.59 (m, 1H), 4.26 (m, 1H), 3.76 (m, 2H), 3.36 (m, 1H), 3.21 (m, 2H), 3.08 (m, 6H), 2.96 (s, 3H).

¹³C NMR (CD₃OD): δ 173.85, 168.04, 162.06, 158.69, 156.92, 152.06, 137.69, 135.05, 131.83, 131.59, 129.77, 128.44, 128.26, 126.21, 123.17, 119.04, 118.75, 57.04, 54.99, 52.08, 51.66, 43.36, 37.24, 36.83, 36.66.

### EXAMPLE 171

### Synthesis of N-(2,4-Difluorobenzenesulfonyl)-L-(1,1-dioxothiamorpholin-3-carbonyl)-L-4-(N,N-dimethylcarbamyloxy)phenylalanine

The title compound was prepared from the product of Example 92 using the procedure described in Method 11.

NMR data was as follows:

¹H NMR (CD₃OD): δ 8.88 (m, ½H), 8.14 (m, ½H), 7.90 (m, 1H), 7.64 (m, 1H), 7.20 (m, 2H), 7.10 (m, 1H), 7.03 (m, 2H), 5.16 (m, 1H), 4.63 (m, 1H), 4.28 (m, 1H), 3.75 (m, 2H), 3.41 (m, 1H), 3.15 (m, 5H), 3.02 (m, 4H).

¹³C NMR (CD₃OD): δ 173.91, 168.04, 156.93, 152.05, 135.15, 133.81, 133.67, 131.60, 123.13, 113.48, 113.18, 107.38, 107.02, 57.02, 55.02, 52.29, 51.84, 43.45, 37.34, 36.83, 36.66.

### EXAMPLE 172

### Synthesis of N-(Toluene-4-sulfonyl)-L-(thiamorpholin-3-carbonyl)-L-4-(N,N-dimethylcarbamyloxy)phenylalanine

The title compound was prepared from the product of Example 49 using the procedure described in Method 11.

NMR data was as follows:

¹H NMR (CDCl₃): δ 7.67 (d, 2H), 7.32 (d, 2H), 7.21 (d, 2H), 7.10 (d, 1H), 7.00 (d, 2H), 5.40 (bs, 1H), 4.85 (m, 2H), 3.95 (m, 1H), 3.41 (m, 1H), 3.07 (m, 6H), 2.98 (m, 4H), 2.62 (m, 1H), 2.41 (m, 5H), 2.13 (m, 1H).

¹³C NMR (CDCl₃): δ 173.40, 168.49, 155.26, 144.44, 136.88, 132.95, 130.51, 130.30, 127.28, 122.08, 55.34, 53.45, 43.43, 36.62, 36.38, 35.85, 25.25, 24.54, 21.43.

### EXAMPLE 173

### Synthesis of N-(Pyridine-3-sulfonyl)-L-(5,5-dimethyl-thiaprolyl-L-4-(N,N-dimethylcarbamyloxy)phenylalanine Isopropyl Ester

The title compound was prepared following the procedure outlined for the preparation of Example 56 by substitution of the appropriate starting materials.

NMR data was as follows:

¹H NMR (CDCl₃): δ 9.13 (m, 1H), 8.90 (m, 1H), 8.19 (m, 1H), 7.56 (m, 1H), 7.23 (d, 2H), 7.04 (d, 2H), 6.93 (d, 1H), 5.07 (m, 1H), 4.85 (m, 1H), 4.62 (d, 1H), 4.48 (d, 1H), 3.92 (s, 1H), 3.20-3.05 (m, 2H), 3.12 (s, 3H), 3.03 (s, 3H), 1.32-1.16 (m, 12H).

¹³C NMR (CDCl₃): δ 170.30, 167.75, 154.19, 150.67, 148.59, 135.72, 132.94, 132.72, 130.27, 123.91, 121.78, 73.62, 69.64, 54.69, 53.12, 50.48, 37.50, 36.53, 36.29, 29.05, 23.73, 21.54, 21.46.

### EXAMPLE 174

### Synthesis of N-(3-Fluorobenzenesulfonyl)-L-(1,1-dioxothiamorpholin-3-carbonyl)-L-4-(N,N-dimethylcarbamyloxy)phenylalanine

The title compound was prepared from the product of Example 91 using the procedure described in Method 11.

NMR data was as follows:

¹H NMR (CD₃OD): δ 7.68 (m, 3H), 7.44 (m, 1H), 7.20 (m, 2H), 7.01 (m, 2H), 5.21 (m, 1H), 4.60 (m, 1H), 4.20 (m, 1H), 3.75 (m, 1H), 3.43 (m, 1H), 3.21 (m, 3H), 3.02 (m, 4H), 2.96 (m, 4H).

¹³C NMR (CD₃OD): δ 173.98, 167.98, 165.89, 162.56, 156.94, 152.06, 142.70, 142.61, 135.11, 133.30, 133.19, 131.57, 124.71, 123.25, 122.21, 121.93, 116.05, 115.71, 57.27, 54.87, 54.79, 51.29, 51.06, 43.24, 37.11, 36.83.

### EXAMPLE 175

### Synthesis of N-(1-Methylpyrazole-4-sulfonyl)-L-(1,1-dioxothiamorpholin-3-carbonyl)-L-4-(N,N-dimethylcarbamyloxy)phenylalanine

The title compound was prepared from the product of Example 169 using the procedure described in Method 11.

NMR data was as follows:

¹H NMR (CD₃OD): δ 8.11 (s, 1H), 7.83 (s, 1H), 7.36 (d, 1H), 7.24 (d, 2H), 7.02 (d, 2H), 5.16 (m, 1H), 4.69 (m, 1H), 4.19 (m, 1H), 3.90 (s, 3H), 3.81 (m, 2H), 3.33 (m, 3H), 3.10 (s, 3H), 3.02 (m, 4H).

¹³C NMR (CD₃OD): 6174.07, 168.11, 156.93, 152.08, 140.12, 135.05, 134.90, 131.67, 123.28, 121.82, 57.33, 54.77, 50.83, 50.64, 42.94, 39.80, 37.02, 36.84, 36.76.

### EXAMPLE 176

### Synthesis of N-(4-tert-Butylbenzenesulfonyl)-L-(1,1-dioxothiamorpholin-3-carbonyl)-L-4-(N,N-dimethylcarbamyloxy)phenylalanine

The title compound was prepared from the product of Example 88 using the procedure described in Method 11.

NMR data was as follows:

¹H NMR (CD₃OD): δ 7.70 (d, 2H), 7.53 (d, 2H), 7.04 (d, 2H), 6.87 (d, 2H), 5.09 (m, 1H), 4.48 (m, 1H), 3.99 (m, 1H), 3.60 (m, 1H), 2.90 (m, 5H), 2.80 (m, 5H), 1.15 (s, 9H).

¹³C NMR (CD₃OD): δ 173.95, 168.09, 159.33, 156.88, 152.09, 137.52, 135.03, 131.54, 128.68, 128.15, 123.32, 57.27, 54.81, 50.75, 43.04, 36.97, 36.82, 36.65, 36.16, 31.35.

### EXAMPLE 177

### Synthesis of N-(Toluene-4-sulfonyl)-(3,3-dimethyl)prolyl-L-4-(N,N-dimethylcarbamyloxy)phenylalanine

The title compound was prepared from the product of Example 97 using the procedure described in Method 11.

NMR data was as follows:

¹H NMR (CDCl₃): δ 7.77 (d, 1H), 7.75 (d, 1H), 7.42-7.33 (m, 3.5H), 7.27 (d, 1H), 7.19 (d, 0.5H), 7.10 (d, 1H), 7.03 (d, 1H), 5.07-5.00 (m, 0.5H), 4.94-4.87 (m, 0.5), 3.67 (d, 1H), 3.58-3.52 (m, 1H), 3.35-3.25 (m, 1H), 3.19-3.08 (m, 2H), 3.11 (s, 3H), 3.02 (s, 3H), 2.45 (s, 1.5H), 2.43 (s, 1.5H), 1.70-1.57 (m, 1H), 1.34-1.27 (m, 1H), 0.94 (s, 1.5H), 0.75 (s, 1.5H), 0.54 (s, 6H).

¹³C NMR (CDCl₃): δ 174.6, 174.4, 171.8, 171.4, 155.7, 150.5, 150.4, 144.5, 144.4, 133.5, 132.6, 130.9, 130.6, 130.0, 129.9, 128.0, 127.9, 122.2, 122.0, 71.2, 70.9, 53.3, 52.2, 47.3, 47.1, 43.0, 42.7, 38.1, 37.9, 36.6, 36.4, 27.0, 26.8, 23.3, 23.0.

### EXAMPLE 178

### Synthesis of N-(2,5-Dichlorothiophene-3-sulfonyl)-L-prolyl-L-4-(N,N-dimethylcarbamyloxy)phenylalanine

The title compound was prepared from the product of Example 86 using the procedure described in Method 11.

NMR data was as follows:

¹H NMR (CD₃OD): δ 8.10 (d, 1H), 7.25 (d, 2H), 7.20 (s, 1H), 7.0 (d, 2H), 4.65 (m, 1H), 4.35 (m, 1H), 3.55-3.35 (m, 2H), 3.30-3.20 (m, 2H), 3.15-3.00 (m, 4H), 2.95 (s, 3H), 2.05-1.80 (m, 2H), 1.80-1.65 (m, 2H).

¹³C NMR (CD₃OD): δ 174.2, 173.9, 156.9, 151.9, 135.9, 135.5, 132.3, 131.6, 128.9, 128.6, 122.9, 63.1, 54.8, 54.7, 50.3, 37.4, 36.8, 36.7, 32.1, 25.5.

### EXAMPLE 179

### Synthesis of N-(4-Methoxybenzenesulfonyl)-L-prolyl-L-4-(N,N-dimethylcarbamyloxy)phenylalanine

The title compound was prepared from the product of Example 180 using the procedure described in Method 7.

NMR data was as follows:

¹H NMR (CD₃OD): δ 7.78 (d, 2H), 7.27 (d, 2H), 7.09 (d, 2H), 7.02 (d, 2H), 4.71-4.67 (m, 1H), 4.10-4.06 (m, 1H), 3.88 (s, 3H), 3.41-3.31 (m, 1H), 3.28-3.07 (m, 6H), 2.97 (s, 3H), 1.81-1.50 (m, 4H).

¹³C NMR (CD₃OD): δ 168.3, 168.2, 159.2, 150.9, 145.9, 129.5, 125.6, 125.3, 123.5, 116.9, 109.6, 57.2, 50.2, 48.7, 44.6, 31.4, 30.8, 30.6, 25.7, 19.3.

### EXAMPLE 180

### Synthesis of N-(4-Methoxybenzenesulfonyl)-L-prolyl-L-4-(N,N-dimethylcarbamyloxy)phenylalanine Isopropyl Ester

The title compound was prepared using the procedure described in Example 2 by substitution of the appropriate starting materials.

NMR data was as follows:

¹H NMR (CDCl₃): δ 7.76 (d, 2H), 7.34 (d, 1H), 7.14 (d, 2H), 7.03-6.97 (m, 4H), 5.08-5.04 (m, 1H), 4.77 (m, 1H), 4.05-4.03 (m, 1H), 3.86 (s, 3H), 3.37-3.34 (m, 1H), 3.26-3.19 (m, 1H), 3.10-3.01 (m, 4H), 2.98 (s, 3H), 2.02 (m, 1H), 1.56-1.46 (m, 3H), 1.25 (d, 6H).

¹³C NMR (CDCl₃): δ 170.8, 170.3, 163.4, 154.8, 150.5, 132.9, 130.1, 129.9, 127.6, 121.6, 114.3, 69.4, 62.1, 55.4, 53.2, 49.5, 37.1, 36.5, 36.2, 29.7, 24.0, 21.5, 21.4.

### EXAMPLE 181

### Synthesis of N-(Toluene-4-sulfonyl)-L-(1-oxo-thiomorpholin-3-carbonyl)thiaprolyl-L-4-(N,N-dimethylcarbamyloxy)phenylalanine

The title compound was prepared from the product of Example 182 using the procedure described in Method 11.

NMR data was as follows:

¹H NMR (CD₃OD): δ 7.90 (m, 1H), 7.78 (m, 2H), 7.40 (m, 2H), 7.26 (m, 2H), 7.03 (m, 2H), 5.14 (m, 1H), 4.64 (m, 2H), 3.81 (m, 1H), 3.71 (m, 2H), 3.19 (m, 1H), 3.14 (m, 3H), 3.02 (m, 4H), 2.84 (m, 1H), 2.60 (m, 1H), 2.42 (m, 4H), 2.21 (m, 1H).

¹³C NMR (CD₃OD): δ 174.22, 173.93, 169.59, 156.88, 152.08, 152.05, 146.44, 146.26, 137.75, 137.63, 135.61, 134.96, 131.79, 131.64, 131.55, 131.39, 128.75, 128.66, 123.35, 123.06, 57.03, 54.88, 54.66, 51.64, 42.69, 42.51, 40.34, 37.12, 36.83, 36.66, 32.76, 21.51.

### EXAMPLE 182

### Synthesis of N-(Toluene-4-sulfonyl)-L-(1-oxo-thiomorpholin-3-carbonyl)-L-4-(N,N-dimethylcarbamyloxy)phenylalanine tert-Butyl Ester

The title compound was prepared following the procedure described for the preparation of Example 49. The oxidation of the thiomorpholine group to the 1-oxo-thiomorpholine group was per Larsson and Carlson (Acta Chemica Scan. 1994, 48, 517-525).

NMR data was as follows:

¹H NMR (CDCl₃): δ 7.72 (m, 2H), 7.69 (m, 2H), 7.31 (m, 2H), 7.11 (m, 2H), 7.07 (m, 2H), 6.96 (m, 2H), 4.79 (m, 1H), 4.54 (m, 1H), 3.80 (m, 4H), 3.04 (4H), 2.92 (m, 3H), 2.64 (m, 1H), 2.43 (m, 4H), 1.44 (s, 3H), 1.36 (s, 6H).

¹³C NMR (CDCl₃): δ 169.8, 166.5, 166.3, 154.6, 150.5, 150.4, 144.9, 144.4, 135.7, 135.3, 132.8, 130.5, 130.1, 29.9, 127.4, 126.9, 122.1, 121.4, 82.6, 82.2, 55.6, 53.9, 53.1, 50.6, 48.1, 47.8, 41.7, 40.5, 38.3, 36.4, 36.1, 31.1, 27.5, 21.2.

### EXAMPLE 183

### Synthesis of N-(3,4-Difluorobenzenesulfonyl)-L-prolyl-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.72-7.60 (m, 2H), 7.87-7.37 (m, 1H), 7.13-7.11 (m, 3H), 7.01 (d, 2H), 5.08-5.04 (m, 1H), 4.81-4.74 (m, 1H), 4.09-4.06 (m, 1H), 3.39-3.35 (m, 1H), 3.26-3.19 (m, 1H), 3.12-2.97 (m, 8H), 2.06-2.03 (m, 1H), 1.66-1.57 (m, 3H), 1.26 (d, 6H).

¹³C NMR (CDCl₃): δ 170.50, 170.40, 154.90, 153.60, 150.70, 150.30, 133.30, 132.90, 130.10, 125.00, 121.80, 121.80, 118.50, 112.80, 69.60, 62.20, 53.20, 49.60, 37.10, 36.60, 36.30, 30.10, 24.20, 21.59, 21.56.

### EXAMPLE 184

### Synthesis of N-(3,4-Difluorobenzenesulfonyl)-L-prolyl-4-(N,N-dimethylcarbamyloxy)phenylalanine

The title compound was prepared from the product of Example 183 using the procedure described in Method 7.

NMR data was as follows:

¹H NMR (CD₃OD): δ 8.10 (d, 1H), 7.84-7.77 (m, 1H), 7.69-7.65 (m, 1H), 7.53-7.45 (m, 1H), 7.28 (d, 2H), 7.02 (d, 2H), 4.72-4.68 (m, 1H), 4.19-4.16 (m, 1H), 3.43-3.39 (m, 1H), 3.31-3.21 (m, 2H), 3.13-3.05 (m, 4H), 2.97 (s, 3H), 1.86-1.61 (m, 4H).

¹³C NMR (CD₃OD): δ 174.2, 174.1, 164.7, 156.9, 154.9, 152.0, 151.6, 135.8, 135.6, 131.6, 129.7, 122.9, 119.7, 118.8, 63.1, 54.7, 50.5, 37.4, 36.8, 36.6, 31.9, 25.5.

### EXAMPLE 185

### Synthesis of N-(3,4-Difluorobenzenesulfonyl)-L-(1,1-dioxothiamorpholin-3-carbonyl)-L-4-(N,N-dimethylcarbamyloxy)phenylalanine tert-Butyl Ester

The title compound was prepared using the procedure described in Example 92 by substitution of the appropriate starting materials.

NMR data was as follows:

¹H NMR (CDCl₃): δ 7.71 (m, 2H), 7.33 (m, 1H), 7.07 (d, 2H), 6.91 (d, 2H), 6.36 (d, 1H), 4.95 (m, 1H), 4.61 (m, 1H), 4.03 (m, 2H), 3.16 (m, 2H), 3.13 (m, 4H), 3.07 (m, 1H), 2.93 (s, 9H), 1.43 (s, 9H).

¹³C NMR (CDCl₃): δ 170.07, 169.45, 164.42, 155.06, 155.44, 154.81, 152.21, 152.17, 150.58, 148.81, 148.64, 134.90, 134.85, 132.41, 130.29, 124.82, 124.71, 124.66, 121.97, 119.07, 118.76, 117.52, 117.23, 82.92, 55.98, 53.20, 50.10, 49.40, 41.76, 36.41, 36.16, 35.99, 27.64.

### EXAMPLE 186

### Synthesis of N-(3,4-Difluorobenzenesulfonyl)-L-(1,1-dioxothiamorpholin-3-carbonyl)-L-4-(N,N-dimethylcarbamyloxy)phenylalanine

The title compound was prepared from the product of Example 185 using the procedure described in Method 11.

NMR data was as follows:

¹H NMR (CD₃OD): δ 6.22 (m, 1H), 6.03 (m, 1H), 5.84 (m, 1H), 5.58 (m, 2H), 5.38 (m, 2H), 3.33 (m, 1H), 3.01 (m, 1H), 2.57 (m, 1H), 2.14 (m, 1H), 1.91 (m, 1H), 1.66 (m, 3H), 1.44 (s, 3H), 1.35 (m, 3H), 1.32 (s, 3H).

¹³C NMR (CD₃OD): δ 173.97, 167.89, 156.94, 153.53, 152.07, 150.00, 137.48, 135.17, 131.63, 126.54, 126.43, 123.20, 120.21, 119.96, 118.84, 118.57, 57.25, 54.82, 51.29, 49.86, 43.29, 37.21, 36.85, 36.67.

### EXAMPLE 187

### Synthesis of N-(Toluene-4-sulfonyl)-L-(5,5-dimethyl)thiaprolyl-L-(thiomorpholin-4-ylcarbonyloxy)phenylalanine tert-Butyl Ester

The title compound was prepared using the procedure described in Example 82 by substitution of the appropriate starting materials.

NMR data was as follows:

¹H NMR (CDCl₃): δ 7.64 (d, 2H), 7.33 (d, 2H), 7.25 (d, 2H), 7.08-6.97 (m, 3H), 4.76 (m, 1H), 4.57 (d, 1H), 4.38 (d, 1H), 3.83 (s, 1H), 3.95-3.78 (m, 4H), 3.09 (m, 2H), 2.69 (m, 4H), 2.43 (s, 3H), 1.44 (s, 9H), 1.16 (s, 3H), 1.08 (s, 3H).

¹³C NMR (CDCl₃): δ 169.78, 168.36, 153.53, 150.28, 144.84, 133.53, 132.76, 130.51, 130.03, 128.19, 121.58, 82.69, 73.42, 54.56, 53.78, 50.46, 47.05, 46.40, 37.80, 29.06, 27.76, 27.37, 27.04, 23.86, 21.52.

### EXAMPLE 188

### Synthesis of N-(Toluene-4-sulfonyl)-L-(5,5-dimethyl)thiaprolyl-L-(thiomorpholin-4-ylcarbonyloxy)phenylalanine

The title compound was prepared from the product of Example 187 using the procedures described in Method 11.

NMR data was as follows:

¹H NMR (CDCl₃): δ 7.77 (d, 2H), 7.37 (d, 2H), 7.28 (d, 2H), 7.22 (d, 1H), 7.03 (d, 2H), 5.35 (brs, 1H), 4.91 (m, 1H), 4.60 (d, 1H), 4.39 (d, 1H), 3.91 (s, 1H), 3.96-3.28 (m, 4H), 3.30-3.07 (m, 2H), 2.67 (m, 4H), 2.45 (s, 3H), 1.10 (s, 3H), 1.08 (s, 3H).

¹³C NMR (CDCl₃): δ 173.09, 169.45, 153.81, 150.28, 145.02, 133.42, 132.61, 130.60, 130.12, 128.13, 121.86, 73.28, 54.51, 53.31, 50.48, 47.08, 46.47, 36.97, 28.97, 27.35, 27.03, 23.70, 21.52.

### EXAMPLE 189

### Synthesis of N-(1-Methylpyrazole-4-sulfonyl)-L-(5,5-dimethyl)thiaprolyl-L-4-(N,N-dimethylcarbamyloxy)phenylalanine Ethyl Ester

The title compound was prepared following the procedure described for the preparation of Example 117 by substitution of the appropriate starting materials.

NMR data was as follows:

¹H NMR (CDCl₃): δ 7.89 (s, 1H), 7.81 (s, 1H), 7.19 (d, 2H), 7.00 (m, 3H), 4.87 (m, 1H), 4.54 (d, 1H), 4.42 (d, 1H), 4.18 (q, 2H), 3.95 (s, 3H), 3.81 (s, 1H), 3.11 (m, 2H), 3.08 (s, 3H), 2.99 (s, 3H), 1.30 (s, 3H), 1.25 (t, 3H), 1.16 (s, 3H).

¹³C NMR (CDCl₃): δ 170.98, 168.34, 154.91, 150.71, 139.62, 132.88, 130.28, 121.85, 117.71, 73.77, 61.66, 54.80, 53.16, 50.53, 39.64, 37.63, 36.60, 36.36, 28.98, 24.00, 13.92.

### EXAMPLE 190

### Synthesis of N-(Pyridine-3-sulfonyl)-L-(5,5-dimethyl)thiaprolyl-L-4-(N,N-dimethylcarbamyloxy)phenylalanine

The title compound was prepared from the product of Example 191 using the procedure described in Method 11.

NMR data was as follows:

¹H NMR (CDCl₃): δ 9.09 (s, 1H), 8.82 (m, 1H), 8.20 (m, 1H), 7.56 (m, 1H), 7.23 (d, 2H), 7.07 (d, 1H), 5.58 (brs, 1H), 4.83 (m, 1h), 4.56 (m, 2H), 4.07 (s, 1H), 3.14 (m, 2H), 3.07 (s, 3H), 2.99 (s, 3H), 1.26 (s, 3H), 1.18 (s, 3H).

¹³C NMR (CDCl₃): δ 173.04, 168.29, 155.16, 153.39, 150.60, 147.96, 136.43, 133.91, 133.06, 130.66, 130.50, 124.65, 122.14, 121.91, 73.43, 54.58, 53.21, 50.38, 37.18, 36.64, 36.38, 29.25, 23.64.

### EXAMPLE 191

### Synthesis of N-(Pyridine-3-sulfonyl)-L-(5,5-dimethyl)thiaprolyl-L-4-(N,N-dimethylcarbamyloxy)phenylalanine tert-Butyl Ester

The title compound was prepared following the procedure described for the preparation of Example 56 and substitution of appropriate starting materials.

Physical data was as follows:

MS: [M+H]⁺ 593

Anal. Calcd. for C₂₇H₃₆N₄O₇S₂.0.5H₂O: C, 53.88; H, 6.07; N, 9.27. Found: C, 53.98; H, 6.07; N, 9.27.

### EXAMPLE 192

### Synthesis of N-(Pyridine-2-sulfonyl)-L-prolyl-L-4-(N,N-dimethylcarbamyloxy)phenylalanine Isopropyl Ester

Substituting 2-pyridinesulfonyl chloride (see Corey et al, J. Org. Chem. 1989, 54, 389-393) and following the method for the preparation of Example 56, gave the title compound.

NMR data was as follows:

¹H NMR (CDCl₃): δ 8.59 (d, 1H), 8.00-7.89 (m, 2H), 7.78 (d, 1H), 7.53-7.49 (m, 1H), 7.16 (d, 2H), 7.01 (d, 2H), 5.05-4.99 (m, 1H), 4.85-4.78 (m, 1H), 4.60-4.57 (m, 1H), 3.44-3.35 (m, 2H), 3.25-3.19 (m, 1H), 3.07 (s, 3H), 3.06-3.01 (m, 1H), 2.97 (s, 3H), 2.19-2.13 (m, 1H), 1.88-1.71 (m, 2H), 1.55 (m, 1H), 1.22-1.19 (m, 6H).

¹³C NMR (CDCl₃): δ 170.90, 170.30, 156.20, 154.80, 150.50, 150.00, 138.00, 133.10, 130.10, 127.00, 123.40, 121.60, 69.20, 62.80, 53.30, 49.60, 37.20, 36.40, 36.20, 29.80, 24.30, 21.42, 21.40.

### EXAMPLE 193

### Synthesis of N-(Pyridine-2-sulfonyl)-L-prolyl-L-4-(N,N-dimethylcarbamyloxy)phenylalanine

The title compound was prepared from the product of Example 192 using the procedure described in Method 7.

NMR data was as follows:

¹H NMR (CD₃OD): δ 8.67 (d, 1H), 8.27 (d, 1H), 8.07-8.02 (m, 1H), 7.96-7.91 (m, 1H), 7.65-7.61 (m, 1H), 7.27 (d, 2H), 7.01 (d, 2H), 4.72-4.69 (m, 1H), 4.58-4.54 (m, 1H), 3.44-3.37 (m, 2H), 3.28-3.24 (m, 1H), 3.13-3.05 (m, 4H), 2.96 (s, 3H), 1.94-1.89 (m, 2H), 1.70-1.63 (m, 2H).

¹³C NMR (CD₃OD): δ 174.5, 174.4, 174.2, 157.7, 156.9, 151.9, 139.9, 135.6, 131.6, 128.8, 124.7, 122.9, 64.1, 54.8, 54.7, 50.9, 37.5, 36.8, 36.7, 31.9, 25.6.

### EXAMPLE 194

### Synthesis of N-(Pyridine-2-sulfonyl)-L-(5,5-dimethyl)thiaprolyl-L-4-(N,N-dimethylcarbamyloxy)phenylalanine Isopropyl Ester

The title compound was prepared following the procedure described for the preparation of Example 192 by substitution of the appropriate starting materials.

NMR data was as follows:

¹H NMR (CDCl₃): δ 8.64-8.62 (m, 1H), 7.98-7.92 (m, 2H), 7.56-7.51 (m, 1H), 7.28-7.21 (m, 3H), 7.01 (d, 2H), 5.01-4.97 (m, 1H), 4.88-4.85 (m, 2H), 4.80 (d, 1H), 4.63 (d, 1H), 4.19 (s, 1H), 3.11-3.07 (m, 5H), 2.98 (s, 3H), 1.28 (s, 3H), 1.26-1.18 (m, 9H).

¹³C NMR (CDCl₃): δ 170.3, 168.4, 155.5, 154.9, 150.7, 150.4, 138.2, 133.0, 130.4, 127.5, 123.5, 121.8, 73.5, 69.5, 54.7, 53.3, 51.0, 37.6, 36.6, 36.4, 29.3, 23.9, 21.52, 21.50.

### EXAMPLE 195

### Synthesis of N-(Pyridine-2-sulfonyl)-L-(5,5-dimethyl)thiaprolyl-L-4-(N,N-dimethylcarbamyloxy)phenylalanine

The title compound was prepared from the product of Example 194 using the procedure described in Method 7.

NMR data was as follows:

¹H NMR (CD₃OD): δ 8.70-8.69 (m, 1H), 8.07-8.01 (m, 1H), 7.92-7.89 (m, 1H), 7.67-7.63 (m, 1H), 4.77-4.67 (m, 3H), 4.30 (s, 1H), 3.23-3.06 (m, 5H), 2.97 (s, 3H), 1.27-1.18 (m, 6H).

¹³C NMR (CD₃OD): δ 174.1, 171.2, 157.0, 151.9, 151.6, 139.9, 135.7, 131.8, 131.7, 129.0, 124.6, 122.9, 74.3, 61.6, 55.7, 54.9, 51.9, 37.6, 36.8, 36.7, 30.1, 24.9.

### EXAMPLE 196

### Synthesis of N-(Toluene-4-sulfonyl)-L-(thiamorpholin-3-carbonyl)-L-4-(N,N-dimethylcarbamyloxy)phenylalanine Isopropyl Ester

The title compound was prepared following the procedure described for the preparation of Example 49 by substitution of the appropriate starting materials.

NMR data was as follows:

¹H NMR (CDCl₃): δ 7.67 (d, 2H), 7.30 (d, 2H), 7.12 (d, 2H), 6.97 (d, 2H), 6.86 (d, 1H), 5.05 (m, 1H), 4.70 (m, 2H), 3.90 (m, 1H), 3.31 (m, 1H), 3.06 (m, 4H), 2.97 (s, 3H), 2.68 (m, 1H), 2.50 (m, 1H), 2.44 (s, 3H), 2.29 (m, 1H), 2.13 (m, 1H), 1.24 (s, 3H), 1.22 (s, 3H).

¹³C NMR (CDCl₃): δ 170.35, 167.55, 155.00, 150.61, 144.20, 136.80, 132.51, 130.24, 130.14, 127.20, 121.82, 69.48, 55.14, 53.55, 43.26, 36.43, 36.16, 25.21, 24.56, 21.48, 21.31.

### EXAMPLE 197

### Synthesis of N-(3-Fluorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.68 (d, 1H), 7.61-7.52 (m, 2H), 7.36 (dt, 1H), 7.21 (d, 2H), 7.02 (d, 2H), 6.94 (d, 1H), 5.05 (sept, 1H), 4.85 (q, 1H), 4.59 (d, 1H), 4.41 (d, 1H), 3.88 (s, 1H), 3.17-3.03 (m, 2H), 3.09 (s, 3H), 3.00 (s, 3H), 1.25 (d, 3H), 1.23 (d, 3H), 1.16 (s, 3H), 1.12 (s, 3H).

¹³C NMR (CDCl₃): δ 170.3, 168.1, 162.6, 154.9, 150.7, 137.9, 132.8, 131.3, 130.4, 123.9, 121.8, 121.0, 115.4, 73.5, 69.6, 54.5, 53.2, 50.5, 37.6, 36.6, 36.3, 29.0, 23.7, 21.6, 21.5.

### EXAMPLE 198

### Synthesis of N-(2-Fluorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.92-7.87 (m, 1H), 7.67-7.59 (m, 1H), 7.33-7.24 (m, 2H), 7.21 (d, 2H), 7.03 (d, 2H), 6.93 (d, 1H), 5.03 (Sept, 1H), 4.83 (q, 1H), 4.67 (d, 1H), 4.63 (d, 1H), 4.03 (s, 1H), 3.16-3.03 (m, 2H), 3.09 (s, 3H), 3.00 (s, 3H), 1.31 (s, 3H), 1.24 (d, 3H), 1.22 (d, 3H), 1.19 (s, 3H).

¹³C NMR (CDCl₃): δ 170.3, 168.1, 159.2, 154.9, 150.7, 136.0, 132.9, 132.0, 130.3, 124.6, 121.8, 117.6, 73.3, 69.6, 54.8, 53.2, 50.3, 37.6, 36.6, 36.3, 29.1, 23.9, 21.6, 21.5.

### EXAMPLE 199

### Synthesis of N-(3,4-Difluorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.77-7.71 (m, 1H), 7.70-7.65 (m, 1H), 7.40-7.31 (m, 1H), 7.20 (d, 2H), 7.02 (d, 2H), 6.87 (d, 1H), 5.05 (Sept, 1H), 4.88-4.82 (m, 1H), 4.55 (d, 1H), 4.44 (d, 1H), 3.91 (s, 1H), 3.17-3.03 (m, 2H), 3.09 (s, 3H), 3.00 (s, 3H), 1.25 (d, 3H), 1.23 (d, 3H), 1.23 (s, 3H), 1.18 (s, 3H).

¹³C NMR (CDCl₃): δ 170.4, 167.9, 154.9, 150.7, 133.1, 132.7, 130.4, 124.4, 121.8, 118.5, 118.0, 73.6, 69.7, 54.6, 53.1, 50.5, 37.6, 36.6, 36.3, 29.2, 23.7, 21.6, 21.5.

### EXAMPLE 200

### Synthesis of N-(3,5-Difluorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.77-7.71 (m, 1H), 7.70-7.65 (m, 1H), 7.40-7.31 (m, 1H), 7.20 (d, 2H), 7.02 (d, 2H), 6.87 (d, 1H), 5.05 (Sept, 1H), 4.88-4.82 (m, 1H), 4.55 (d, 1H), 4.44 (d, 1H), 3.91 (s, 1H), 3.17-3.03 (m, 2H), 3.09 (s, 3H), 3.00 (s, 3H), 1.25 (d, 3H), 1.23 (d, 3H), 1.23 (s, 3H), 1.18 (s, 3H).

¹³C NMR (CDCl₃): δ 170.4, 167.9, 154.9, 150.7, 133.1, 132.7, 130.4, 124.4, 121.8, 118.5, 118.0, 73.6, 69.7, 54.6, 53.1, 50.5, 37.6, 36.6, 36.3, 29.2, 23.7, 21.6, 21.5.

### EXAMPLE 201

### Synthesis of N-(2,4-Difluorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.94-7.86 (m, 1H), 7.20 (d, 2H), 7.03 (d, 2H), 7.02-6.95 (m, 2H), 6.88 (d, 1H), 5.03 (Sept, 1H), 4.82 (q, 1H), 4.67 (d, 1H), 4.61 (d, 1H), 4.01 (s, 1H), 3.16-3.03 (m, 2H), 3.09 (s, 3H), 3.00 (s, 3H), 1.36 (s, 3H), 1.23 (d, 3H), 1.21 (d, 3H), 1.20 (s, 3H).

¹³C NMR (CDCl₃): δ 170.3, 167.9, 154.9, 150.7, 133.7, 132.8, 130.3, 121.8, 112.1, 106.1, 73.4, 69.6, 54.9, 53.2, 50.4, 37.6, 36.6, 36.3, 29.1, 23.9, 21.6, 21.5.

### EXAMPLE 202

### Synthesis of N-(4-Chlorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.82 (d, 2H), 7.53 (d, 2H), 7.21 (d, 2H), 7.02 (d, 2H), 6.93 (d, 1H), 5.05 (Sept, 1H), 4.89-4.82 (m, 1H), 4.55 (d, 1H), 4.41 (d, 1H), 3.87 (s, 1H), 3.17-3.03 (m, 2H), 3.09 (s, 3H), 3.00 (s, 3H), 1.25 (d, 3H), 1.23 (d, 3H), 1.16 (s, 6H).

¹³C NMR (CDCl₃): δ 170.3, 168.1, 154.9, 150.7, 140.4, 134.5, 132.8, 130.4, 129.7, 129.5, 121.8, 73.5, 69.6, 54.6, 53.1, 50.5, 37.6, 36.6, 36.3, 29.1, 23.8, 21.6, 21.0.

### EXAMPLE 203

### Synthesis of N-(3-Chlorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.88 (t, 1H), 7.78-7.75 (m, 1H), 7.64-7.61 (m, 1H), 7.51 (t, 1H), 7.21 (d, 2H), 7.02 (d, 2H), 6.92 (d, 1H), 5.05 (sept, 1H), 4.89-4.82 (m, 1H), 4.58 (d, 1H), 4.40 (d, 1H), 3.88 (s, 1H), 3.18-3.03 (m, 2H), 3.09 (s, 3H), 3.00 (s, 3H), 1.25 (d, 3H), 1.23 (d, 3H), 1.16 (s, 3H), 1.14 (s, 3H).

¹³C NMR (CDCl₃): δ 170.3, 168.0, 154.9, 150.7, 137.7, 135.7, 133.9, 132.8, 130.7, 130.3, 127.9, 126.2, 121.8, 73.6, 69.96, 54.5, 53.2, 50.5, 37.6, 36.6, 36.3, 29.1, 23.7, 21.6, 21.5.

### EXAMPLE 204

### Synthesis of N-(2-Chlorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 8.08 (dd, 1H), 7.54-7.52 (m, 2H), 7.45-7.39 (m, 1H), 7.19 (d, 2H), 7.02 (d, 2H), 6.79 (d, 1H), 5.00 (sept, 1H), 4.78 (d, 1H), 4.75-4.68 (m, 1H), 4.69 (d, 1H), 4.19 (s, 1H), 3.09 (s, 3H), 3.06 (d, 2H), 3.00 (s, 3H), 1.38 (s, 3H), 1.23 (s, 3H), 1.23 (d, 3H), 1.19 (d, 3H).

¹³C NMR (CDCl₃): δ 170.3, 168.1, 154.9, 150.7, 135.6, 134.4, 132.8, 132.7, 132.4, 130.3, 127.3, 121.8, 73.3, 69.5, 54.7, 53.3, 50.4, 37.6, 36.6, 36.3, 29.6, 23.7, 21.6, 21.5.

### EXAMPLE 205

### Synthesis of N-(3,4-Dichlorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.97 (d, 1H), 7.70 (dd, 1H), 7.63 (d, 1H), 7.20 (d, 2H), 7.02 (d, 2H), 6.86 (d, 1H), 5.05 (sept, 1H), 4.89-4.82 (m, 1H), 4.55 (d, 1H), 4.43 (d, 1H), 3.92 (s, 1H), 3.17-3.03 (m, 2H), 3.09 (s, 3H), 3.00 (s, 3H), 1.26 (d, 3H), 1.22 (d, 3H), 1.23 (s, 3H), 1.18 (s, 3H).

¹³C NMR (CDCl₃): δ 170.3, 167.9, 154.9, 150.7, 138.7, 136.1, 134.2, 132.7, 131.4, 130.3, 129.8, 127.1, 121.8, 73.6, 69.7, 54.6, 53.1, 50.5, 37.5, 36.6, 36.3, 29.2, 23.7, 21.6, 21.5.

### EXAMPLE 206

### Synthesis of N-(3,5-Dichlorobenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting material.

NMR data was as follows:

¹H NMR (CDCl₃): δ 7.76 (d, 2H), 7.62 (t, 1H), 7.20 (d, 2H), 7.03 (d, 2H), 6.85 (d, 1H), 5.05 (sept, 1H), 4.89-4.82 (m, 1H), 4.57 (d, 1H), 4.42 (d, 1H), 3.92 (s, 1H), 3.18-3.04 (m, 2H), 3.09 (s, 3H), 3.00 (s, 3H), 1.25 (d, 3H), 1.23 (d, 3H), 1.27 (s, 3H), 1.18 (s, 3H).

¹³C NMR (CDCl₃): δ 170.3, 167.8, 154.9, 150.7, 139.1, 136.5, 133.7, 132.7, 130.3, 126.2, 121.8, 73.7, 69.7, 54.6, 53.1, 50.5, 37.5, 36.6, 36.3, 29.2, 23.7, 21.6, 21.5.

### EXAMPLE 207

### Synthesis of N-(3-Chlorobenzenesulfonyl)-L-(1,1-dioxothiamorpholin-3-carbonyl)-L-4-(N,N-dimethylcarbamyloxy)phenylalanine tert-Butyl Ester

The title compound was prepared using the procedure described in Example 92 by substitution of the appropriate starting materials.

NMR data was as follows:

¹H NMR (CDCl₃): δ 7.85 (m, 1H), 7.76 (m, 1H), 7.63 (m, 1H), 7.53 (m, 1H), 7.06 (d, 2H), 6.96 (d, 2H), 6.37 (m, 1H), 5.01 (m, 1H), 4.62 (m, 1H), 4.01 (m, 2H), 3.26 (m, 1H), 3.06 (s, 3H), 2.96 (m, 7H), 1.49 (s, 9H).

¹³C NMR (CDCl₃): δ 170.0, 164.5, 154.9, 150.6, 140.0, 136.1, 134.2, 132.5, 131.3, 130.2, 127.4, 125.5, 122.2, 82.8, 56.0, 53.3, 49.9, 49.2, 41.7, 36.5, 36.3, 36.0, 27.8.

### EXAMPLE 208

### Synthesis of N-(3,4-Dichlorobenzenesulfonyl)-L-(1,1-dioxothiamorpholin-3-carbonyl)-L-4-(N,N-dimethylcarbamyloxy)phenylalanine tert-Butyl Ester

The title compound was prepared using the procedure described in Example 92 by substitution of the appropriate starting materials.

NMR data was as follows:

¹H NMR (CDCl₃): δ 7.91 (m, 1H), 7.66 (m, 2H), 7.06 (d, 2H), 6.94 (d, 2H), 6.33 (m, 1H), 4.98 (m, 1H), 4.60 (m, 1H), 3.49 (m, 3H), 3.12 (m, 2H), 3.04 (s, 3H), 3.00 (m, 2H), 2.94 (s, 3H), 1.44 (s, 9H).

¹³C NMR (CDCl₃): δ 170.0, 164.3, 154.8, 150.6, 138.8, 137.9, 134.3, 132.4 132.0, 130.3, 129.2, 126.4, 122.1, 83.0, 55.5, 53.1, 50.2, 49.5, 41.8, 36.5, 36.2, 36.0, 27.7.

### EXAMPLE 209

### Synthesis of N-(4-Methoxybenzenesulfonyl)-L-(5,5-dimethyl)thiaprolyl-L-4-(N,N-dimethylcarbamloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.81 (d, 2H), 7.22 (d, 2H), 7.06-6.99 (m, 5H), 5.04 (sept, 1H), 4.89-4.82 (m, 1H), 4.56 (d, 1H), 4.39 (d), 3.88 (s, 3H), 3.83 (s, 1H), 3.17-3.03 (m, 2H), 3.09 (s, 3H), 3.00 (s, 3H), 1.25 (d, 3H), 1.22 (d, 3H), 1.15 (s, 3H), 1.12 (s, 3H).

¹³C NMR (CDCl₃): δ 170.3, 168.5, 163.8, 154.9, 150.7, 132.9, 130.4, 130.3, 127.4, 121.7, 114.5, 73.5, 69.5, 55.6, 54.6, 53.2, 50.5, 37.7, 36.6, 36.3, 29.1, 23.9, 21.6, 21.5.

### EXAMPLE 210

### Synthesis of N-(3-Methoxybenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.47-7.45 (m, 2H), 7.37-7.36 (m, 1H), 7.21 (d, 2H), 7.19-7.15 (m, 1H), 7.04-6.98 (m, 3H), 5.04 (sept, 1H), 4.88-4.82 (m, 1H), 4.58 (d, 1H), 4.40 (d, 1H), 3.89 (s, 1H), 3.87 (s, 3H), 3.17-3.03 (m, 2H), 3.09 (s, 3H), 3.00 (s, 3H), 1.25 (d, 3H), 1.23 (d, 3H), 1.15 (s, 3H), 1.08 (s, 3H).

¹³C NMR (CDCl₃): δ 170.3, 168.3, 160.2, 154.9, 150.7, 136.9, 132.9, 130.5, 130.4, 121.7, 120.2, 120.0, 112.6, 73.4, 69.6, 55.7, 54.5, 53.2, 50.4, 37.7, 36.6, 36.3, 29.1, 23.7, 21.6, 21.5.

### EXAMPLE 211

### Synthesis of N-(2-Methoxybenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.92 (dd, 1H), 7.54 (dd, 1H), 7.21 (d, 2H), 7.07-7.00 (m, 4H), 6.96 (d, 1H), 5.01 (sept, 1H), 4.83-4.76 (m, 1H), 4.73 (d, 1H), 4.61 (d, 1H), 4.17 (s, 1H), 3.93 (s, 3H), 3.14-3.03 (m, 2H), 3.09 (s, 3H), 3.00 (s, 3H), 1.36 (s, 3H), 1.22 (d, 3H), 1.21 (s, 3H), 1.19 (d, 3H).

¹³C NMR (CDCl₃): δ 170.3, 168.7, 157.7, 154.9, 150.6, 135.4, 133.0, 132.5, 130.3, 125.2, 121.7, 120.5, 112.6, 73.3, 69.5, 56.0, 54.8, 53.3, 50.4, 37.7, 36.6, 36.3, 29.2, 24.1, 21.6, 21.5.

### EXAMPLE 212

### Synthesis of N-(3,4-Dimethoxybenzenesulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.50 (dd, 1H), 7.31 (d, 1H), 7.21 (d, 2H), 7.05-7.01 (m, 3H), 6.97 (d, 1H), 5.04 (sept, 1H), 4.89-4.82 (m, 1H), 4.56 (d, 1H), 4.40 (d, 1H), 3.95 (s, 3H), 3.94 (s, 3H), 3.89 (s, 1H), 3.17-3.03 (m, 2H), 3.09 (s, 3H), 3.00 (s, 3H), 1.25 (d, 3H), 1.22 (d, 3H), 1.16 (s, 3H), 1.14 (s, 3H).

¹³C NMR (CDCl₃): δ 170.3, 168.5, 154.9, 153.5, 150.7, 149.4, 132.9, 130.4, 127.6, 122.3, 121.7, 110.6, 110.3, 73.5, 69.6, 56.3, 56.1, 54.6, 53.2, 50.5, 37.7, 36.6, 36.3, 29.2, 23.8, 21.6, 21.5.

### EXAMPLE 213

### Synthesis of N-(2,4-Difluorobenzenesulfonyl)-L-(thiamorpholin-3-carbonyl)-L-4-(N,N-dimethylcarbamyloxy)phenylalanine Isopropyl Ester

The title compound was prepared following the procedure described for the preparation of Example 49 by substitution of the appropriate starting material.

NMR data was as follows:

¹H NMR (CDCl₃): δ 7.89 (m, 1H), 7.16 (m, 2H), 6.97 (m, 4H), 6.77 (d, 1H), 4.72 (m, 1H), 4.60 (m, 1H), 3.92 (m, 1H), 3.29 (m, 1H), 3.09 (m, 5H), 2.93 (s, 3H), 2.70 (m, 2H), 2.55 (m, 1H), 2.10 (m, 1H), 1.42 (s, 9H).

¹³C NMR (CDCl₃): δ 170.0, 168.0, 167.7, 137.1, 164.4, 164.3, 161.1, 160.9, 157.7, 157.5, 154.8, 150.5, 132.7, 132.6, 132.4, 130.4, 124.0, 123.8, 121.7, 112.2, 111.9, 106.5, 106.1, 105.8, 82.6, 55.4, 53.9, 43.5, 36.4, 36.2, 27.7, 26.8, 25.5.

### EXAMPLE 214

### Synthesis of N-(3,4-Dichlorobenzenesulfonyl)-L-(1,1-dioxothiamorpholin-3-carbonyl)-L-4-(N,N-dimethylcarbamyloxy)phenylalanine

The title compound was prepared from the product of Example 208 using the procedure described in Method 11.

NMR data was as follows:

¹H NMR (CD₃OD): δ 8.04 (m, 1H), 7.68 (m, 2H), 7.52 (m, 1H), 7.21 (d, 2H), 7.02 (d, 2H), 5.22 (m, 1H), 4.63 (m, 1H), 4.22 (m, 1H), 3.71 (m, 1H), 3.57 (m, 1H), 3.30 (m, 3H), 3.08 (s, 3H), 3.02 (m, 3H), 2.97 (s, 3H).

¹³C NMR (CD₃OD): δ 174.0, 168.0, 156.9, 152.1, 140.7, 139.3, 135.2, 133.2, 131.6, 130.7, 128.3, 123.2, 57.2, 54.9, 54.6, 51.7, 51.4, 43.3, 37.3, 36.9, 36.7.

### EXAMPLE 215

### Synthesis of N-(3-Chlorobenzenesulfonyl)-L-(1,1-dioxothiamorpholin-3-carbonyl)-L-4-(N,N-dimethylcarbamyloxy)phenylalanine

The title compound was prepared from the product of Example 207 using the procedure described in Method 11.

NMR data was as follows:

¹H NMR (CD₃OD): δ 7.94 (m, 1H), 7.77 (m, 2H), 7.58 (m, 1H), 7.46 (d, 1H), 7.19 (d, 2H), 7.07 (d, 2H), 5.23 (m, 1H), 4.63 (m, 1H), 4.20 (m, 1H), 3.71 (m, 1H), 3.43 (m, 1H), 3.26 (m, 4H), 3.17 (s, 3H), 2.95 (m, 5H).

¹³C NMR (CD₃OD): δ 168.0, 152.1, 142.5, 136.8, 135.0, 132.7, 131.6, 128.6, 127.1, 123.3, 57.2, 54.9, 51.4, 51.2, 43.2, 37.2, 36.8, 36.7.

### EXAMPLE 216

### Synthesis of N-(3-Chloro-4-fluorobenzenesulfonyl)-L-(1,1-dioxothiamorpholin-3-carbonyl)-L-4-(N,N-dimethylcarbamyloxy)phenylalanine tert-Butyl Ester

The title compound was prepared using the procedure described in Example 92 by substitution of the appropriate starting materials.

NMR data was as follows:

¹H NMR (CD₃OD): δ 7.93 (d), 7.90 (m), 7.29 (s), 7.27 (d), 7.04 (d), 4.60 (m), 4.46 (d), 3.90-3.40 (m), 3.10 (s), 2.98 (s), 1.43 (s).

¹³C NMR (CD₃OD): δ 171.5, 166.5, 156.9, 151.9, 135.2, 131.3, 129.9, 127.9, 127.8, 123.1, 117.8, 117.5, 101.4, 83.7, 57.9, 56.0, 42.9, 37.3, 36.9, 36.7, 28.1.

### EXAMPLE 217

### Synthesis of N-(1-Methylpyrazole-4-sulfonyl)-L-(thiamorpholin-3-carbonyl)-L-4-(N,N-dimethylcarbamyloxy)phenylalanine tert-Butyl Ester

The title compound was prepared following the procedures described for the preparation of Examples 49 and 117.

NMR data was as follows:

¹H NMR (CDCl₃): δ 7.77 (s), 7.63 (s), 7.08 (d), 6.93 (d), 6.76 (d), 6.71 (d), 5.50 (d), 5.22 (s), 4.82 (t), 4.61 (q), 3.83 (s), 3.25 (dt), 3.04 (m), 2.90 (s), 2.05 (dd), 1.34 (s).

¹³C NMR (CDCl₃): δ 169.3, 166.8, 154.7, 150.4, 138.4, 132.4, 132.2, 130.2, 121.4, 118.3, 105.4, 82.5, 55.2, 53.6, 53.3, 39.5, 38.3, 36.6, 36.3, 36.1, 27.6, 23.5.

### EXAMPLE 218

### Synthesis of N-(3,4-Difluorobenzenesulfonyl)-L-(thiamorpholin-3-carbonyl)-L-4-(N,N-dimethylcarbamyloxy)phenylalanine tert-Butyl Ester

The title compound was prepared following the procedure described for the preparation of Example 49 by substitution of the appropriate starting materials.

NMR data was as follows:

¹H NMR (CD₃OD): δ 7.88 (m, 1H), 7.70 (m, 1H), 7.57 (m, 1H), 7.23 (d, 2H), 7.03 (d, 2H), 6.83 (d, 1H), 5.63 (dd, 1H), 5.07 (t, 1H), 4.58 (m, 1H), 3.22-3.00 (m, 3H), 3.09 (s, 3H), 2.98 (s, 3H), 2.07 (dd, 1H), 1.44 (s, 9H).

¹³C NMR (CD₃OD): δ 171.3, 169.3, 156.9, 152.0, 135.0, 131.6, 126.5, 122.9, 120.2, 119.9, 119.4, 118.7, 118.4, 106.4, 83.6, 56.5, 55.6, 37.1, 36.8, 36.6, 28.1, 25.2.

### EXAMPLE 219

### Synthesis of N-(Toluene-4-sulfonyl)-L-(5,5-dimethyl)thioprolyl-L-(thiomorpholin-4-ylcarbonyloxy)phenylalanine Isopropyl Ester

The title compound was prepared using the procedure described in Example 82 by substitution of the appropriate starting materials.

NMR data was as follows:

¹H NMR (CDCl₃): δ 9.09 (s, 1H), 8.88 (m, 1H), 8.16 (m, 1H), 7.50 (m, 1H), 7.22 (d, 2H), 7.01 (d, 2H), 6.91 (d, 1H), 5.05 (m, 1H), 4.85 (m, 1H), 4.60 (d, 1H), 4.46 (d, 1H), 3.89 (s, 1H), 3.93-3.83 (m, 4H), 3.11 (m, 2H), 2.69 (m, 4H), 1.29-1.16 (m, 12H).

¹³C NMR (CDCl₃): δ 170.3, 167.8, 154.3, 153.5, 150.4, 148.7, 135.8, 133.1, 132.9, 130.4, 124.0, 121.8, 73.7, 69.7, 54.7, 53.2, 50.5, 47.1, 46.4, 37.6, 29.1, 27.4, 27.0, 23.8, 21.6, 21.5.

### EXAMPLE 220

### Synthesis of N-(3,4-Difluorobenzenesulfonyl)-L-(thiamorpholin-3-carbonyl)-L-4-(N,N-dimethylcarbamyloxy)phenylalanine

The title compound was prepared from the product of Example 218 using the procedure described in Method 11.

NMR data was as follows:

¹H NMR (CDCl₃): δ 7.89 (s, 1H), 7.57-7.46 (m, 2H), 7.35 (d, 1H), 7.32-7.22 (m, 1H), 7.09 (d, 2H), 6.91 (d, 2H), 6.64 (d, 1H), 5.50 (d, 1H), 4.89 (s, 1H), 4.88-4.79 (m, 1H), 3.17-3.02 (m, 3H), 3.02 (s, 3H), 2.93 (s, 3H), 1.75 (dd, 1H).

¹³C NMR (CDCl₃): δ 173.6, 167.7, 155.5, 152.0, 151.8, 150.1, 148.4, 132.8, 130.4, 124.6, 121.5, 118.7, 118.5, 117.5, 117.3, 117.1, 106.9, 54.9, 53.0, 36.4, 36.2, 36.0, 23.4.

### EXAMPLE 221

### Synthesis of N-(2,5-Dichlorothiophene-3-sulfonyl)-L-(5,5-dimethyl)thiaprolyl-L-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 7.18 (d, 2H), 7.11 (s, 1H), 7.00 (d, 2H), 6.87 (d, 1H), 5.03-4.99 (m, 1H), 4.84-4.81 (m, 1H), 4.65-4.56 (m, 2H), 4.07 (s, 1H), 3.10-3.01 (m, 5H), 2.98 (s, 3H), 1.37 (s, 3H), 1.22 (s, 3H), 1.21 (s, 3H), 1.18 (s, 3H).

¹³C NMR (CDCl₃): δ 170.3, 167.7, 154.9, 150.7, 132.9, 132.8, 131.9, 130.3, 128.0, 127.0, 121.8, 73.4, 69.6, 54.8, 53.2, 50.5, 37.5, 36.6, 36.3, 29.1, 23.8, 21.6, 21.5.

### EXAMPLE 222

### Synthesis of N-(1-Methylpyrazole-4-sulfonyl)-L-(5,5-dimethyl)thiaprolyl-L-4-(thiomorpholin-4-ylcarbonyloxy)phenylalanine Isopropyl Ester

The title compound was prepared using the procedure described in Example 82 by substitution of the appropriate starting materials.

NMR data was as follows:

¹H NMR (CDCl₃): δ 7.89 (s, 1H), 7.80 (s, 1H), 7.21 (d, 2H), 7.01 (m, 3H), 5.03 (m, 1H), 4.83 (m, 1H), 4.54 (d, 1H), 4.40 (d, 1H), 3.95 (s, 3H), 3.86 (m, 4H), 3.80 (s, 1H), 3.09 (m, 2H), 2.68 (m, 4H), 1.28 (s, 3H), 1.22 (m, 6H), 1.16 (s, 3H).

¹³C NMR (CDCl₃): δ 170.4, 168.3, 153.5, 150.4, 139.3, 133.3, 132.9, 130.4, 121.7, 117.6, 73.8, 69.7, 54.8, 53.2, 50.5, 47.1, 46.4 39.6, 37.6, 29.0, 27.4, 27.1, 24.0, 21.6, 21.5.

### EXAMPLE 223

### Synthesis of N-(8-Quinolinesulfonyl)-L-prolyl-L-4-(N,N-dimethylcarbamyloxy)phenylalanine Isopropyl Ester

The title compound was prepared following the procedure described for the preparation of Example 2 by substitution of the appropriate starting materials.

NMR data was as follows:

¹H NMR (CDCl₃): δ 9.01-8.99 (m, 1H), 8.56-8.53 (m, 1H), 8.27-8.23 (m, 1H), 8.07-8.04 (m, 1H), 7.66-7.61 (m, 2H), 7.55-7.51 (m, 1H), 7.17 (d, 2H), 7.01 (d, 2H), 5.27-5.23 (m, 1H), 5.07-4.98 (m, 1H), 4.84-4.76 (m, 1H), 3.34-3.20 (m, 3H), 3.06-2.98 (m, 4H), 2.97 (s, 3H), 2.15-2.09 (m, 1H), 1.64-1.51 (m, 3H), 1.23 (d, 6H).

¹³C NMR (CDCl₃): δ 172.0, 170.5, 154.9, 151.5, 150.6, 143.9, 136.8, 135.6, 134.9, 134.1, 133.3, 130.2, 129.2, 125.6, 122.3, 121.7, 69.3, 62.8, 53.5, 48.7, 37.3, 36.5, 36.3, 29.7, 24.3, 21.6, 21.6.

### EXAMPLE 224

### Synthesis of N-(8-Quinolinesulfonyl)-L-prolyl-L-4-(N,N-dimethylcarbamyloxy)phenylalanine

The title compound was prepared from the product of Example 223 using the procedure described in Method 7.

NMR data was as follows:

¹H NMR (CD₃OD): δ 9.03-9.01 (m, 1H), 8.49-8.42 (m, 2H), 8.23-8.20 (m, 1H), 8.09-8.07 (m, 1H), 7.73-7.61 (m, 2H), 7.25 (d, 2H), 7.00 (d, 2H), 5.30-5.27 (m, 1H), 4.73-4.69 (m, 1H), 3.38-3.21 (m, 3H), 3.09-3.02 (m, 4H), 2.95 (s, 3H), 1.86 (m, 1H), 1.78-1.73 (m, 1H), 1.58-1.50 (m, 2H).

¹³C NMR (CD₃OD): δ 175.3, 174.2, 164.7, 156.9, 152.9, 145.2, 138.5, 136.9, 135.8, 135.6, 131.6, 130.9, 126.9, 123.8, 122.9, 63.9, 54.7, 50.0, 37.5, 36.8, 36.7, 31.6, 25.5.

### EXAMPLE 225

### Synthesis of N-(8-Quinolinesulfonyl)-L-(5,5-dimethyl)thiaprolyl-L-4-(N,N-dimethylcarbamyloxy)phenylalanine Isoproplyl Ester

The title compound was prepared following the procedure described for the preparation of Example 2 by substitution of the appropriate starting materials.

NMR data was as follows:

¹H NMR (CDCl₃): δ 9.05-9.03 (m, 1H), 8.53-8.49 (m, 1H), 8.26-8.22 (m, 1H), 8.08-8.05 (m, 1H), 7.65-7.60 (m, 1H), 7.56-7.52 (m, 1H), 7.19 (d, 2H), 7.06 (d, 1H), 7.00 (d, 2H), 5.17 (d, 1H), 4.94 (m, 1H), 7.74-4.78 (m, 2H), 4.66 (s, 1H), 3.08-2.99 (m, 8H), 1.20-1.16 (m, 12H).

¹³C NMR (CDCl₃): δ 170.2, 168.9, 154.9, 151.5, 150.6, 144.2, 136.7, 134.4, 134.4, 133.1, 130.3, 129.2, 125.5, 122.3, 121.7, 73.2, 69.3, 54.8, 53.3, 50.6, 37.6, 36.6, 36.3, 29.2, 24.1, 21.5, 21.4.

### EXAMPLE 226

### Synthesis of N-(8-Quinolinesulfonyl)-L-(5,5-dimethyl)thiaprolyl-L-4-(N,N-dimethylcarbamyloxy)phenylalanine

The title compound was prepared from the product of Example 225 using the procedure described in Method 7.

NMR data was as follows:

¹H NMR (CD₃OD): δ 9.06-9.04 (m, 1H), 8.45-8.39 (m, 2H), 8.23-8.14 (m, 1H), 7.72-7.61 (m, 2H), 7.32 (d, 2H), 7.03 (d, 2H), 5.12 (d, 1H), 4.87 (d, 1H), 4.69-4.64 (m, 2H), 3.28-3.02 (m, 5H), 2.98 (s, 2H), 1.18 (s, 3H), 1.08 (s, 3H).

¹³C NMR (CD₃OD): δ 174.1, 171.8, 157.1, 152.9, 152.0, 145.5, 138.4, 137.3, 135.8, 135.6, 135.1, 131.8, 130.9, 126.8, 123.8, 122.9, 73.7, 55.9, 54.8, 51.7, 37.6, 36.8, 36.7, 30.2, 25.0.

### EXAMPLE 227

### Synthesis of N-(3-Sulfonamido-4-chloro-benzenesulfonyl)-L-prolyl-L-4-(N,N-dimethylcarbamyloxy)phenylalanine Isopropyl Ester

The title compound was prepared following the procedure described for the preparation of Example 2 and substitution of appropriate starting materials.

NMR data was as follows:

¹H NMR (CDCl₃): δ 8.45 (d, 1H), 7.91 (d, 1H), 7.67 (d, 1H), 7.13 (d, 2H), 7.06 (d, 1H), 7.01 (d, 2H), 5.90 (brs, 2H), 5.06-5.02 (m, 1H), 4.79-4.72 (m, 1H), 4.14-4.10 (m, 1H), 3.42-3.39 (m, 1H), 3.25-3.14 (m, 2H), 3.07 (s, 3H), 3.04-2.97 (m, 1H), 2.96 (s, 3H), 1.98-1.96 (m, 1H), 1.72-1.62 (m, 3H), 1.28-1.25 (m, 6H).

¹³C NMR (CDCl₃): δ 170.8, 170.7, 155.1, 150.6, 141.4, 136.9, 136.1, 132.9, 132.8, 131.9, 130.3, 128.7, 121.9, 69.8, 62.1, 53.3, 49.6, 36.9, 36.6, 36.4, 30.4, 24.3, 21.6, 21.6.

### EXAMPLE 228

### Synthesis of N-(Toluene-4-sulfonyl)-L-(1-oxothiomorpholin-3-carbonyl)-L-4-(N,N-dimethylcarbamyloxy)phenylalanine Isopropyl Ester

The title compound was prepared following the procedure described for the preparation of Example 182 and substitution of appropriate starting materials.

NMR data was as follows:

¹H NMR (CDCl₃): δ 7.77 (d, 2H), 7.72 (d, 2H), 7.33 (m, 2H), 7.20 (m, 2H), 7.12 (d, 2H), 7.01 (m, 2H), 5.10 (m, 1H), 5.01 (m, 1H), 4.84 (m, 1H), 4.75 (m, 1H), 3.80 (m, 3H), 3.05 (m, 4H), 2.96 (m, 3H), 2.74 (m, 1H), 2.42 (m, 4H), 1.30-1.20 (m, 6H).

¹³C NMR (CDCl₃): δ 170.6, 170.4, 166.8, 166.7, 154.9, 150.7, 150.6, 145.1, 144.8, 135.8, 135.5, 132.7, 130.6, 130.4, 130.3, 130.0, 127.7, 127.1, 122.4, 121.8, 69.8, 69.4, 55.8, 53.7, 52.9, 50.8, 48.2, 47.9, 42.0, 41.2, 38.4, 36.6, 36.5, 36.3, 31.2, 21.5, 21.5.

### EXAMPLE 229

### Synthesis of N-(2,4-Difluorobenzenesulfonyl)-L-(1-oxothiomorpholin-3-carbonyl)-L-4-(N,N-dimethylcarbamyloxy)phenylalanine tert-Butyl Ester

The title compound was prepared following the procedure described for the preparation of Example 182 and substitution of appropriate starting materials.

NMR data was as follows:

¹H NMR (CDCl₃): δ 7.91 (m, 1H), 7.30 (m, 2H), 6.97 (m, 4H), 4.71 (m, 1H), 4.55 (m, 1H), 3.90 (m, 2H), 3.77 (m, 1H), 3.11 (m, 4H), 2.85 (m, 3H), 2.80 (m, 1H), 2.60 (m, 2H), 1.46 (s, 9H), 1.39 (s, 9H).

¹³C NMR (CDCl₃): δ 170.0, 168.0, 167.9, 166.4, 166.2, 164.6, 164.4, 162.7, 161.4, 161.2, 157.9, 157.8, 154.8, 150.6, 150.4, 132.8, 132.5, 132.4, 130.9, 130.4, 130.1, 123.3, 123.1, 122.2, 121.6, 121.1, 122.6, 122.2, 111.9, 106.6, 106.3, 105.9, 82.8, 82.3, 55.8, 54.1, 53.2, 51.6, 49.2, 48.7, 43.1, 42.3, 38.7, 36.5, 36.2, 31.8, 27.7.

### EXAMPLE 230

### Synthesis of N-(1-Methylpyrazole-4-sulfonyl)-L-(5,5-dimethyl)thiaprolyl-L-4-(N,N-dimethylcarbamyloxy)phenylalanine 2,2-Dimethylpropyl Ester

The product from Example 161 (1 g., 0.72 mmol) was dissolved in neopentyl alcohol (5 mL). Titanium (IV) isopropoxide (260 mg., 0.9 mmol) was added and the mixture heated at 100 C under an inert atmosphere for 48 h. Excess neopentyl alcohol was removed under reduced pressure and the residue purified by flash column chromatography (silica, 1% MeOH in CHCl₃) to give the title compound as a white solid (1.02 g, 97%).

Physical data was as follows:

MS (+) ESI [M+H]⁺ 610; [M+NH₄]⁺ 627 (100%).

Anal. Calcd. for C₂₉H₃₉N₅O₇S: C, 53.18; H, 6.45; N, 11.49. Found: C, 53.46; H, 6.38; N, 11.06.

### EXAMPLE 231

### Synthesis of N-(Pyridine-3-sulfonyl)-L-(5,5-dimethyl)thiaprolyl-L-4-(N,N-dimethylcarbamyloxy)phenylalanine 2,2-Dimethylpropyl Ester

The product from Example 173 was subjected to the transesterification procedure described for the preparation of Example 230. The compound was purfied by flash column chromatography (silica, 1% MeOH in CHCl₃) followed by recrystallization from ethyl acetate to give the title compound as a white solid (720 mg, 47%).

Physical data was as follows:

Anal. Calcd. for C₂₈H₃₈N₄O₇S₂: C, 55.43; H, 6.31; N, 9.23. Found: C, 55.37; H, 6.32; N, 9.22.

### EXAMPLE 232

### Synthesis of N-(1-Methylpyrazole-4-sulfonyl)-L-(5,5-dimethyl)thiaprolyl-L-4-(N,N-dimethylcarbamyloxy)phenylalanine Cyclopropylmethyl Ester

The product from Example 161 was subjected to the transesterification procedure described for the preparation of Example 230. The title compound was obtained as a white solid following flash column chromatography (silica, 1% MeOH in CHCl₃) (860 mg, 70%).

Physical data was as follows:

Anal. Calcd. for C₂₆H₃₅N₅O₇S₂: C, 52.6; H, 5.94; N, 11.8. Found: C, 52.49; H, 5.93; N, 11.62.

### EXAMPLE 233

### Synthesis of N-(1-Methylpyrazole-4-sulfonyl)-L-(5,5-dimethyl)thiaprolyl-L-4-(N,N-dimethylcarbamyloxy)phenylalanine Methyl Ester

The title compound was prepared following the procedure described for the preparation of Example 161 and substitution of appropriate starting materials.

Physical data was as follows:

MS (+) ESI [M+H]⁺ 554; [M+NH₄]⁺ 571 (100%).

Anal. Calcd. for C₂₃H₃₁N₅O₇S₂.0.2 EtOAc: C, 50.04; H, 5.75; N, 12.26. Found: C, 50.12; H, 5.69; N, 12.19.

### EXAMPLE 234

### Synthesis of N-(Pyridine-3-sulfonyl)-L-(5,5-dimethyl)thiaprolyl-L-4-(N,N-dimethylcarbamyloxy)phenylalanine Ethyl Ester

The product from Example 173 was subjected to the transesterification procedure described for the preparation of Example 230. The compound was purified by flash column chromatography (silica, 2% MeOH in CHCl₃), followed by recrystallization from ethyl acetate to give the title compound as a white solid (1.2 g, 61%).

Physical data was as follows:

Anal. Calcd. for C₂₅H₃₂N₄O₇S₂: C, 53.18; H, 5.71; N, 9.92. Found: C, 53.14; H, 5.72; N, 9.57.

### EXAMPLE 235

### Synthesis of N-(Pyridine-3-sulfonyl)-L-(5,5-dimethyl)thiaprolyl-L-4-(N,N-dimethylcarbamyloxy)phenylalanine Cyclopropylmethyl Ester

The product from Example 173 was subjected to the transesterification procedure described for the preparation of Example 230. The compound was isolated as a white solid following flash column chromatography (silica, 2% MeOH in CHCl₃) and recrystallization from EtOAc/hexanes (1 g, 65%).

Physical data was as follows:

Anal. Calcd. for C₂₇H₃₄N₄O₄S₂: C, 54.9; H, 5.8; N, 9.48. Found: C, 54.77; H, 5.65; N, 9.46.

### EXAMPLE 236

### Synthesis of N-(1-Methylpyrazole-4-sulfonyl)-L-(5,5-dimethyl)thiaprolyl-L-4-(N,N-dimethylcarbamyloxy)phenylalanine 2-Methoxyphenyl Ester

To a solution of the compound from Example 139 (1.79 g, 3.31 mmol), 2-methoxy-phenol (0.45 g, 3.64 mmol) and BOP (1.61 g, 3.64 mmol) in methylene chloride (25 mL) at 0° C. was added triethylamine (0.7 mL, 4.97 mmol). The reaction mixture was then slowly warmed to 25° C. where it was stirred, under nitrogen, for 24 h. The reaction was quenched by addition of 100 mL saturated brine and extracted with EtOAc. The organic extract was washed sequentially with 2N HCl (3 times), saturated sodium bicarbonate (3×) and saturated brine (2×), dried over MgSO₄, and evaporated to 2.1 g of crude product. Flash chromatography (eluant: 96-4 methylene chloride:EtOAc) afforded 1.85 g of a white solid which upon trituration with hexane gave 1.68 g (79%) of white crystals, mp 72-75° C.

Physical data was as follows:

Anal. Calcd. for C₂₉H₃₅N₅O₈S₂: C, 59.94; H, 5.46; N, 10.85. Found: C, 53.45; H, 5.62; N, 10.31.

### EXAMPLE 237

### Synthesis of N-(1-Methylpyrazole-4-sulfonyl)-L-(5,5-dimethyl)thiaprolyl-L-4-(N,N-dimethylcarbamyloxy)phenylalanine n-Butyl Ester

A solution of the compound of Example 139 (2 g) in n-butanol (50 mL) was saturated upon ice-cooling with HCl gas. The mixture was stirred at ambient temperature for 36 h, evaporated in vacuo to almost dryness, then partitioned between 5% NaHCO₃ and chloroform. The organic layer was dried and evaporated in vacuo to furnish 900 mg of the title compound.

Physical data was as follows:

MS: [(+) ESI], [M+H]⁺ 596.

### EXAMPLE 238

### Synthesis of N-(1-Methylpyrazole-4-sulfonyl)-L-(5,5-dimethyl)thiaprolyl-L-4-(N,N-dimethylcarbamyloxy)phenylalanine n-Propyl Ester

A solution of N-(1-methylpyrazole-4-sulfonyl)-L-(5,5-dimethyl)thiaprolyl-L-4-(N,N-dimethylcarbamyloxy)phenylalanine (2 g) in n-propanol (50 mL) was saturated upon ice-cooling with HCl gas. The mixture was stirred at ambient temperature for 36 hours, evaporated in vacuo to almost dryness, then partioned between 5% NaHCO₃ and chloroform. The organic layer was dried and evaporated in vacuo to provide 1500 mg of the title compound.

Physical data was as follows:

MS: [(+) ESI], [M+H]⁺ 582.

### EXAMPLE 239

### Synthesis of N-(1-Methylpyrazole-4-sulfonyl)-L-(5,5-dimethyl)thiaprolyl-L-4-(N,N-dimethylcarbamyloxy)phenylalanine 2,2-Dimethylpropionyloxymethyl Ester

Potassium iodide (324 mg) was added at once to a mixture of the compound of Example 139 (1.08 g), chloromethylpivalate (294 mg) and powdered K₂CO₃ (222 mg) in DMF (5 mL). The reaction mixture was stirred at ambient temperature overnight, partitioned between water (12 mL) and ethyl acetate (60 mL). The separated organic layer was washed with ice cold 0.1 N sodium thiosulfate, water and brine, then dried over MgSO₄, filtered and evaporated in vacuo to yield 750 mg of the title compound.

Physical data was as follows:

MS: [(+) ESI], [M+H]⁺ 654.

### EXAMPLE 240

### Synthesis of N-(Toluene-4-sulfonyl)-L-prolyl-L-4-(4-phenylpiperazin-1-ylcarbonyloxy)phenylalanine tert-Butyl Ester

The title compound was prepared following the procedure outlined for Example 4 by substitution of the appropriate starting materials. A white solid was obtained, mp. 60-65° C.

Physical data was as follows:

MS (+ESI) 694.3 [M+NH₄]⁺.

Anal. Calcd for C₃₆H₄₄N₄O₇S 0.5C₄H₈O₂: C, 63.31; H, 6.71; N, 7.77. Found: C, 63.12; H, 6.58; N, 7.69.

### EXAMPLE 241

### Synthesis of N-(Toluene-4-sulfonyl)-L-prolyl-L-4-(4′-(ethoxycarbonyl)piperidin-1-ylcarbonyloxy)phenylalanine tert-Butyl Ester

The carbamate was prepared by treatment of Tos-Pro-Tyr-t-butyl ester with 4-nitrophenylchloroformate, followed by addition of ethylisonipecotate (triethylamine, methylene chloride, chilled to 0° C., then stirred at room temperature overnight). The crude product was purified by flash chromatography (silica, 95:5 EtoAc:Et₃N) to afford a white solid. (0.78 g, 39%).

NMR data was as follows:

¹H NMR (DMSO-d₆, 400 MHz): δ 8.15 (d, 1H, J=7.68 Hz); 7.70 (d, 2H, J=8.34 Hz); 7.40 (d, 2H, J=7.90 Hz); 7.22 (d, 2H, J=8.56 Hz); 7.00 (d, 2H, J=8.56 Hz); 4.37 (m, 1H), 4.07 (q, 2H, J=7.14, 14.08 Hz); 4.03 (m, 2H); 3.90 (m, 1H); 3.34 (m, 1H); 3.09 (m, 2H); 3.00 (m, 3H); 2.59 (m, 1H); 2.39 (s, 3H); 1.87 (m, 2H); 1.58 (m, 5H); 1.41 (m, 1H); 1.35 (s, 9H); 1.18 (t, 3H, 7.14 Hz).

IR (KBr, cm⁻¹): 3410, 2990, 2950, 1725, 1680, 1510, 1430, 1355, 1220, 1200, 1170, 1000, 675, 595.

MS ((+) ESI, m/z (%)) 689 (100[M+NH₄]⁺); 691 (37[M+NH₄]⁺).

Anal. Calcd. for C₃₄H₄₅N₃O₉S: C, 60.79; H, 6.75; N, 6.25. Found: C, 60.59; H, 6.67; N, 6.22.

### EXAMPLE 242

### Synthesis of N-(Toluene-4-sulfonyl)-L-prolyl-L-4-(N-(4′-(2′-aminoethyl)morpholino)carbamyloxy)phenylalanine

The title compound was prepared from the product of Example 152 using the procedure described in Method 11.

NMR data was as follows:

¹H NMR (DMSO-d₆, 400 MHz): δ 12.75 (s, 1H); 8.08 (d, 1H); 7.68 (d, 2H); 7.60 (t, 1H); 7.39 (d, 2H); 7.21 (d, 2H); 6.97 (d, 2H); 4.46 (m, 1H); 4.08 (m, 1H); 3.56 (m, 4H); 3.26 (m, 3H); 3.09 (m, 2H); 2.94 (m, 1H); 2.49 (s, 6H); 2.48 (s, 3H); 1.5 (m, 3H); 1.38 (m, 1H).

IR (KBr, cm⁻¹) 3400, 2975, 1725, 1650, 1500, 1350, 1150, 650, 575, 550.

MS ((−) ESI, m/z (%)) 587 (100[M−H]⁺).

Anal. Calcd. for C₂₈H₃₆N₄O₈S.HCOOH.0.5H₂O: C, 54.11; H, 6.11; N, 8.70. Found: C, 53.96; H, 6.02; N, 8.68.

### EXAMPLE 243

### Synthesis of N-(Toluene-4-sulfonyl)-L-prolyl-L-4-[4-(carboxy)piperidin-1-ylcarbonyloxy]phenylalanine

The title compound was prepared from the product of Example 241 using the procedures described in Methods 6 and 11.

NMR data was as follows:

¹H NMR (DMSO-d₆, 400 MHz): δ 12.50 (bs, 2H); 8.08 (d, 1H, J=7.90 Hz); 7.69 (d, 2H, J=8.34 Hz); 7.39 (d, 2H, J=7.90 Hz); 7.22 (D, 2H, J=8.56 Hz); 6.99 (d, 2H, J=8.56 Hz); 4.46 (m, 1H); 4.09 (m, 1H); 4.00 (m, 1H); 3.90 (m, 1H); 3.30 (m, 1H); 3.09 (m, 3H); 2.95 (m, 2H); 2.49 (m, 1H); 2.38 (s, 3H); 1.86 (m, 2H); 1.36-1.61 (m, 6H).

IR (KBr, cm⁻¹) 3400, 2960, 1720, 1535, 1430, 1350, 1200, 1160, 670, 590, 550.

MS ((+) ESI, m/z (%)) 605 (100[M+NH₄]⁺).

Anal. Calcd. for C₂₈H₃₃N₃O₉S H₂O: C, 55.53; H, 5.65; N, 6.94. Found: C, 55.23; H, 5.82; N, 6.59.

### EXAMPLE 244

### Synthesis of N-(Toluene-4-sulfonyl)-L-prolyl-L-4-(N,N-bis-(2-hydroxyethyl)carbamyloxy)phenylalanine Isopropyl Ester

The carbamate was prepared by treatment of Tos-Pro-Tyr-iPr ester with 4-nitrophenyl chloroformate, followed by addition of diethanol amine (triethylamine, methylene chloride, chilled to 0° C., stirred at room temperature overnight). The crude product was purified by flash chromatography (silica, 98:2 EtOAc:EtOH) to afford a white foam. (0.180 g, 28%).

NMR data was as follows:

¹H NMR (DMSO-d₆, 400 MHz): δ 8.26 (d, 1H, J=7.90 Hz); 7.69 (d, 2H, J=8.12 Hz); 7.40 (d, 2H, J=8.12 Hz); 7.23 (D, 2H, J=8.56 Hz); 6.99 (d, 2H, J=8.56 Hz); 4.87 (m, 1H); 4.83 (t, 1H, J=5.49 Hz); 4.76 (t, 1H, J=5.49 Hz); 4.42 (m, 1H); 4.08 (m, 1H); 3.58 (m, 2H); 3.51 (m, 2H); 3.44 (m, 2H); 3.34 (m, 3H); 2.99-3.09 (m, 3H); 2.39 (s, 3H); 1.59 (m, 3H); 1.41 (m, 1H); 1.16 (d, 3H, J=6.15 Hz); 1.12 (d, 3H, J=6.15 Hz).

IR (KBr, cm⁻¹) 3420, 2940, 1725, 1535, 1670, 1520, 1460, 1410, 1350, 1220, 1160, 1110, 670, 600, 550.

MS ((+) ESI, m/z (%)) 606 (15[M+H]⁺); 623 (100[M+NH₂]⁺).

Anal. Calcd. for C₂₉H₃₉N₃O₉S H₂O: C, 56.66; H, 6.56; N, 6.84. Found: C, 56.66; H, 6.41; N, 6.72.

### EXAMPLE 245

### Synthesis of N-(Toluene-4-sulfonyl)-L-prolyl-L-4-[3-(hydroxymethyl)piperidin-1-ylcarbonyloxy]phenylalanine Isopropyl Ester

The carbamate was prepared by treatment of Tos-Pro-Tyr-iPr ester with 4-nitrophenyl chloroformate, followed by addition of 3-piperidine methanol (triethylamine, methylene chloride, chilled to 0° C., stirred at room temperature overnight). The crude product was purified by flash chromatography (silica, 3:2 EtOAc:Hex) to afford a white foam (0.519 g, 67%).

NMR data was as follows:

¹H NMR (DMSO-d₆, 400 MHz): δ 8.26 (d, 1H, J=7.90 Hz); 7.69 (d, 2H, J=8.12 Hz); 7.40 (d, 2H, J=8.12 Hz); 7.22 (d, 2H, J=8.56 Hz); 6.98 (d, 2H, J=8.34 Hz); 4.85 (M, 1H); 4.57 (bs, 1H); 4.42 (m, 1H); 3.99-4.09 (m, 3H); 3.85 (m, 1H); 3.31 (m, 1H); 3.22 (m, 1H); 2.91-3.10 (m, 4H); 2.80 (m, 1H); 2.55 (m, 1H); 2.39 (s, 3H); 1.51-1.72 (m, 6H); 1.42 (m, 2H); 1.16 (d, 3H, J=6.15 Hz); 1.11 (d, 3H), J=6.15 Hz).

IR (KBr, cm⁻¹) 3400, 2990, 2940, 2880, 1725, 1520, 1430, 1350, 1220, 1165, 1100, 660, 600, 550.

MS ((−) ESI, m/z (%)) 614 (30[M−H]).

Anal. Calcd. for C₃₁H₄₁N₃O₈S: C, 60.47; H, 6.71; N, 6.82. Found: C, 59.83; H, 6.61; N, 6.59.

### EXAMPLE 246

### Synthesis of N-(Toluene-4-sulfonyl)-L-prolyl-L-4-(4-trifluoromethanesulfonylpiperazin-1-ylcarbonyloxy)phenylalanine tert-Butyl Ester

The title compound was prepared following the procedure outlined for Example 128 and substitution of appropriate starting materials.

Physical data was as follows:

MS (+ESI): 733 [M+H]⁺.

Anal. Calcd. for C₃₁H₃₉F₃N₄O₉S₂.0.10C₄H₈O₂: C, 50.20; H, 5.40; N, 7.55. Found: C, 50.25; H, 5.46; N, 7.07.

### EXAMPLE 247

### Synthesis of N-(4-(N-Phenylurea)benzenesulfonyl)-L-prolyl-L-4-(N,N-dimethylcarbamyloxy)phenylalanine tert-Butyl Ester

A mixture of Example 107 (250 mg, 0.51 mmol), phenyl isocyanate (62 mg, 0.56 mmol) and triethylamine (76 μL, 0.56 mmol) was heated to reflux under argon. Reflux was continued overnight. Solvent was removed under reduced pressure and the residue purified by flash chromatography. (silica, hexanes: EtOAc 1:1 then EtOAc) to give the title compound as an off-white foam (160 mg, 46%), mp 112-115° C.

Physical data was as follows:

MS (+ESI) [M+NH₄]⁺ 697 (100%).

### EXAMPLE 248

### Synthesis of N-(2-Trifluoroacetyl-1,2,3,4-tetrahydroisoquinolin-7-sulfonyl)-L-prolyl-L-4-(N,N-dimethylcarbamyloxy)phenylalanine Isopropyl Ester

The title compound was prepared following the procedure described for the preparation of Example 2 and substitution of appropriate starting materials.

NMR data was as follows:

¹H NMR (CDCl₃): δ 7.70-7.66 (m, 2H), 7.35-7.30 (m, 1H), 7.27-7.21 (m, 1H), 7.14-7.10 (m, 2H), 7.01 (d, 2H), 5.09-4.95 (m, 1H), 4.89-4.75 (m, 2H), 4.14-4.07 (m, 1H), 3.93-3.85 (m, 2H), 3.35-3.20 (m, 2H), 3.13-2.97 (m, 9H), 2.05-2.01 (m, 1H), 1.63 (1.50 (m, 3H), 1.20 (d, 6H).

¹³C NMR (CDCl₃): δ 170.7, 170.6, 170.5, 156.3, 155.8, 154.9, 150.6, 140.1, 139.2, 135.1, 135.1, 13.2, 133.0, 133.0, 132.9, 130.2, 130.1, 129.9, 126.9, 126.4, 126.3, 125.8, 121.7, 118.3, 114.5, 69.6, 62.1, 62.0, 53.2, 49.6, 46.6, 46.5, 45.1, 42.7, 40.9, 37.1, 36.6, 36.3, 30.1, 30.0, 29.2, 27.8, 24.2, 24.2, 21.6, 21.6.

### EXAMPLE 249

### Synthesis of N-(1-Methylpyrazole-3-sulfonyl)-L-(5,5-dimethyl)thiaprolyl-L-4-(N,N-dimethylcarbamyloxy)phenylalanine Isopropyl Ester

Substituting N-methylpyrazole-3-sulfonyl chloride (See European Patent Application, 095925) and following the method for the preparation of Example 56, gave the title compound.

NMR data was as follows:

¹H NMR (CDCl₃): δ 7.45 (d, 1H), 7.21 (d, 2H), 7.09 (d, 1H), 7.01 (d, 2H), 6.71 (d, 1H), 5.03-4.98 (m, 1H), 4.87-4.84 (m, 1H), 4.60-4.59 (m, 2H), 4.05 (s, 1H), 3.97 (s, 3H), 3.12-3.01 (m, 5H), 2.98 (s, 3H), 1.22-1.15 (m, 12H).

¹³C NMR (CDCl₃): δ 170.3, 168.3, 154.9, 150.7, 146.7, 133.0, 131.9, 130.3, 121.7, 108.9, 73.5, 69.5, 54.7, 53.3, 50.7, 39.9, 37.7, 36.6, 36.3, 28.8, 24.1, 21.5, 21.5.

### EXAMPLE 250

### Synthesis of N-(1-Methylpyrazole-3-sulfonyl)-L-(5,5-dimethyl)thiaprolyl-L-4-(N,N-dimethylcarbamyloxy)phenylalanine

The title compound was prepared from the product of Example 249 using the procedure described in Method 7.

NMR data was as follows:

¹H NMR (CD₃OD): δ 8.25 (d, 1H), 7.76 (d, 1H), 7.32 (d, 2H), 7.01 (d, 2H), 6.70 (d, 1H), 4.74-4.71 (m, 1H), 4.68 (d, 1H), 4.56 (d, 1H), 4.12 (s, 1H), 3.97 (s, 3H), 3.24-3.07 (m, 5H), 2.97 (s, 3H), 1.14 (s, 3H), 1.13 (s, 3H).

¹³C NMR (CD₃OD): δ 174.1, 171.4, 157.0, 151.9, 148.2, 135.7, 134.2, 131.8, 122.9, 109.6, 74.4, 55.6, 55.0, 51.5, 40.0, 37.6, 36.8, 36.7, 29.6, 24.8.

### EXAMPLE 251

### Synthesis of N-(Pyridine-4-sulfonyl)-L-(5,5-dimethyl)thiaprolyl-L-4-(N,N-dimethylcarbamyloxy)phenylalanine Isopropyl Ester

The title compound was prepared following the procedure outlined for the preparation of Example 56, where 4-pyridinesulfonyl chloride N-oxide was used in place of 3-pyridinesulfonyl chloride (see Marsais and coworkers, J. Org. Chem. 1987, 52, 1133-1136). Deoxygenation of the N-oxide was accomplished using the procedure of Aoyagi and coworkers, Synthesis 1997, 891.

NMR data was as follows:

¹H NMR (CDCl₃): δ 8.89-8.87 (m, 2H), 7.72-7.70 (m, 2H), 7.19 (d, 2H), 7.01 (d, 2H), 6.79 (d, 1H), 5.05-5.01 (m, 1H), 4.85-4.82 (m, 1H), 4.58 (d, 1H), 4.45 (d, 1H), 3.91 (s, 1H), 3.11-3.02 (m, 5H), 2.99 (s, 3H), 1.28-1.16 (m, 12H).

¹³C NMR (CDCl₃): δ 170.3, 167.7, 154.9, 151.5, 150.7, 144.2, 132.7, 130.3, 121.8, 120.9, 73.6, 69.7, 54.6, 53.1, 50.4, 37.5, 36.6, 36.3, 29.1, 23.6, 21.6, 21.5.

### EXAMPLE 252

### Synthesis of N-(Pyridine-4-sulfonyl)-L-(5,5-dimethyl)thiaprolyl-L-4-(N,N-dimethylcarbamyloxy)phenylalanine

The title compound was prepared from the product of Example 251 using the procedure described in Method 7.

NMR data was as follows:

¹H NMR (CD₃OD): δ 8.78 (d, 2H), 7.42 (d, 1H), 7.69 (d, 2H), 7.35 (d, 2H), 7.06 (d, 2H), 4.69-4.61 (m, 3H), 4.16 (s, 1H), 3.25-3.19 (m, 1H), 3.13-3.05 (m, 4H), 2.97 (s, 3H), 1.25 (s, 6H).

¹³C NMR (CD₃OD): δ 174.1, 170.5, 157.0, 152.2, 152.0, 147.2, 135.8, 131.8, 123.1, 122.7, 73.9, 55.6, 54.9, 54.4, 37.5, 36.8, 36.7, 30.1, 24.8.

### EXAMPLE 253

### Synthesis of N-(Toluene-4-sulfonyl)-L-(5,5-dimethyl)thiaprolyl-L-4-(N-methyl-N-(2-dimethylaminoethyl)carbamyloxy)phenylalanine tert-Butyl Ester

A solution of the starting acid (500 mg), (2S)-2-amino-3-{4-[(2-dimethylaminoethyl)-methylcarbamoyloxy]phenyl}propionic acid tert-butyl ester (730 mg), HOBt (235 mg), and 4-methylmorpholine (0.87 mL) in DMF (10 mL) was stirred in ice bath at 0° C. 1-[3-(Dimethylamino)propyl]-3-ethylcarbodiimide hydrochloride (360 mg) was added to the solution. The ice bath was removed after 10 minutes. The reaction was stirred at room temperature for 3 hours. Ethyl acetate (20 mg) was added. The solution was washed with saturated NaHCO₃ solution (30 mL) 2 times, then washed with brine. The solution was dried with MgSO₄. The solvent was evaporated in vacuo, and the residue flash chromatographed on silica gel to give 385 mg of the title compound.

Physical data was as follows:

MS: [(+) ESI], [M+H]⁺ 663.

### EXAMPLE 254

### Synthesis of N-(Toluene-4-sulfonyl)-L-prolyl-L-4-(N-methyl-N-(2-dimethylaminoethyl)carbamyloxy)phenylalanine tert-Butyl Ester

The title compound was prepared following the procedure described for the preparation of Example 253 and substitution of appropriate starting materials.

Physical data was as follows:

MS: [(+) ESI], [M+H]⁺ 617.

### EXAMPLE 255

### Synthesis of N-(Toluene-4-sulfonyl)-L-(5,5-dimethyl)thiapropyl-L-4-(N-methyl-N-(2-dimethylaminoethyl)carbamyloxy)phenylalanine

The title compound was prepared from the product of Example 253 using the procedure described in Method 11.

Physical data was as follows:

MS: [(+) ESI], [M+H]⁺ 607.

### EXAMPLE 256

### Synthesis of N-(Toluene-4-sulfonyl)-L-prolyl-L-4-(N-methyl-N-(2-dimethylaminoethyl)carbamyloxy)phenylalanine

The title compound was prepared from the product of Example 254 using the procedure described in Method 11.

Physical data was as follows:

MS: [(+) ESI], [M+H]⁺ 561.

### EXAMPLE 257

### Synthesis of N-(4-Fluorobenzenesulfonyl)-L-(5,5-dimethyl)thiaprolyl-L-3-chloro-4-(4-methylpiperazin-1-ylcarbonyloxy)phenylalanine tert-Butyl Ester

The title compound was prepared following the procedure described for the preparation of Example 4 and substitution of appropriate starting materials, mp: 64-67° C.

Physical data was as follows:

MS: [M+H]⁺ 699.

Anal. Calcd. for C₃₁H₄₀ClFN₄O₇S₂H₂O: C, 51.90; H, 5.9; N, 7.8. Found: C, 51.53; H, 5.50; N, 7.62.

### EXAMPLE 258

### Synthesis of N-(4-Fluorobenzenesulfonyl)-L-(5,5-dimethyl)thiaprolyl-L-3-chloro-4-(N,N-dimethycarbamyloxy)phenylalanine

The title compound was prepared for the product of Example 257 using the procedure described in Method 11.

Physical data was as follows:

MS: [M+1] 603.

Anal. Calcd. for C₂₄H₂₇FN₃O₇S₂: C, 49.02; H, 4.63; N, 7.15. Found: C, 49.25; H, 4.89; N, 6.73.

### EXAMPLE 259

### Synthesis of N-(4-Fluorobenzenesulfonyl)-L-(5,5-dimethyl)thiaprolyl-L-3-chloro-4-(thiomorpholin-4-ylcarbonyloxy)phenylalanine tert-Butyl Ester

The title compound was prepared following the procedure described for the preparation of Example 82 and substitution of appropriate starting materials, mp. 111-114° C.

Physical data was as follows:

MS: +ESI [M+NH₄]⁺ 719.

Anal. Calcd. for C₃₀H₃₇ClFN₃O₇S: C, 50.02; H, 5.46; N, 5.8. Found: C, 50.23; H, 5.10; N, 5.50.

### EXAMPLE 260

### Synthesis of N-(4-Fluorobenzenesulfonyl)-L-(5,5-dimethyl)thiaprolyl-L-3-chloro-4-(thiomorpholin-4-ylcarbonyloxy)phenylalanine Isopropyl Ester

The title compound was prepared following the procedure described for the preparation of Example 82 and substitution of appropriate starting materials, mp. 77-81° C.

Physical data was as follows:

MS: [M+NH₄]+ 705.

Anal. Calcd. for C₂₉H₃₅ClFN₃O₇S₃: C, 50.61; H, 5.13; N, 6.1. Found: C, 50.33; H, 5.07; N, 5.94.

### EXAMPLE 261

### Synthesis of N-(4-Fluorobenzenesulfonyl)-L-(5,5-dimethyl)thiaprolyl-L-3-chloro-4-(N,N-dimethylcarbamyloxy)]phenylalanine Isopropyl Ester

The title compound was prepared following the procedure described for the preparation of Example 2 and substitution of appropriate starting materials, mp. 65-69° C.

Physical data was as follows:

MS: [M+NH₄]⁺ 647.

Anal. Calcd. for C₂₇H₃₃ClFN₃O₇S₂: C, 51.46; H, 5.28; N, 6.4. Found: C, 51.29; H, 5.19; N, 6.50.

### EXAMPLE 262

### Synthesis of N-(Toluene-4-sulfonyl)-L-(5,5-dimethyl)thiaprolyl-L-3-chloro-4-(N,N-dimethylcarbamyloxy)phenylalanine Isopropyl Ester

The title compound was prepared following the procedure described for the preparation of Example 2 and substitution of appropriate starting materials, mp. 68-72° C.

Physical data was as follows:

MS: [M+H]⁺ 626.

Anal. Calcd. for C₂₈H₃₆ClN₃O₇S₂: C, 53.77; H, 5.80; N, 6.71. Found: C, 53.26; H, 5.8; N, 6.63.

### EXAMPLE 263

### Synthesis of N-(4-Fluorobenzenesulfonyl)-L-(5,5-dimethyl)thiaprolyl-L-3-chloro-4-(4-methylpiperazin-1-ylcarbonyloxy)]phenylalanine Isopropyl Ester

The title compound was prepared following the procedure described for the preparation of Example 4 and substitution of appropriate starting materials.

Physical data was as follows:

MS: [M+H]⁺ 685.

Anal. Calcd. for C₃₀H₃₈ClN₄O₇: C, 52.59; H, 5.59; N, 8.18. Found: C, 52.09; H, 5.48; N, 7.77.

### EXAMPLE 264

### Synthesis of N-(Toluene-4-sulfonyl)-L-prolyl-L-3-chloro-4-(N,N-dimethylcarbamyloxy)]phenylalanine Isopropyl Ester

The title compound was prepared following the procedure described for the preparation of Example 2 and substitution of appropriate starting materials.

Physical data was as follows:

MS: [M+H]⁺ 580.

Anal. Calcd. for C₂₇H₃₄ClN₃O₇S 0.5H₂O: C, 55.04; H, 6.00; N, 7.13. Found: C, 55.06; H, 5.71; N, 6.93.

### EXAMPLE 265

### Synthesis of N-(4-Fluorobenzenesulfonyl)-L-(5,5-dimethyl)thiaprolyl-L-3-chloro-4-(4-(2′-pyridyl)-piperazin-1-ylcarbonyloxy)]phenylalanine Isopropyl Ester

The title compound was prepared following the procedure described for the preparation of Example 4 and substitution of appropriate starting materials.

Physical data was as follows:

MS: [M+H]⁺ 748.

Anal. Calcd. for C₃₄H₃₉ClFN₅O₇S₂: C, 54.57; H, 5.25; N, 9.3. Found: C, 54.26; H, 5.10; N, 9.07.

### EXAMPLE 266

### Synthesis of N-(4-Fluorobenzenesulfonyl)-L-(5,5-dimethyl)thiaprolyl-L-3-chloro-4-(4-(2′-pyridyl)-piperazin-1-ylcarbonyloxy)]phenylalanine tert-Butyl Ester

The title compound was prepared following the procedure described for the preparation of Example 4 and substitution of appropriate starting materials, mp. 80-86° C.

Physical data was as follows:

MS: [M+H]⁺ 762.

Anal. Calcd. for C₃₅H₄₁ClFN₅O₇S₂: C, 55.14; H, 5.42; N, 9.19. Found: C, 54.67; H, 5.40; N, 8.69.

### EXAMPLE 267

### Synthesis of N-(4-Nitrobenzenesulfonyl)-L-prolyl-L-4-(N,N-dimethylcarbamyloxy)phenylalanine Isopropyl Ester

The title compound was prepared following the procedure described for the preparation of Example 2 and substitution of appropriate starting materials.

Physical data was as follows:

Anal. Calcd. for C₂₆H₃₂N₄O₉S: C, 54.16; H, 5.59; N, 9.72. Found: C, 53.69; H, 5.24; N, 9.52.

### EXAMPLE 268

### Synthesis of N-(4-Aminobenzenesulfonyl)-L-prolyl-L-4-(N,N-dimethylcarbamyloxy)phenylalanine Isopropyl Ester

The title compound was prepared from the product of Example 267 using the procedure described in Method 4.

Physical data was as follows:

Anal. Calcd. for C₂₆H₃₄N₄O₇S: C, 57.13; H, 6.27; N, 10.25. Found: C, 56.30; H, 6.12; N, 10.05.

### EXAMPLE 269

### Synthesis of N-(Toluene-4-sulfonyl)-L-prolyl-L-4-(thiomorpholin-4-ylcarbonyloxy)phenylalanine Isopropyl Ester

The title compound was prepared following the procedure described for the preparation of Example 82 and substitution of appropriate starting materials.

Physical data was as follows:

Anal. Calcd. for C₂₉H₃₇N₃O₇S₂: C, 57.69; H, 6.18; N, 6.96. Found: C, 57.36; H, 5.99; N, 6.76.

### EXAMPLE 270

### Synthesis of N-(Toluene-4-sulfonyl)-L-prolyl-L-4-(4-phenylcarbamylpiperazin-1-ylcarbonyloxy)phenylalanine Isopropyl Ester

The title compound was prepared following the procedure described for the preparation of Example 4 and substitution of appropriate starting materials.

NMR data was as follows:

¹H NMR (DMSO-d₆, 400 MHz): δ 8.62 (s, 1H); 8.11 (d, 1H); 7.73 (d, 2H); 7.45 (m, 4H); 7.26 (m, 3H); 7.04 (m, 2H); 6.95 (m, 1H); 6.25 (d, 1H); 4.90 (m, 1H); 4.50 (m, 1H); 4.11 (m, 1H); 3.6 (br, 4H); 3.4 (br, 4H); 3.10 (m, 2H); 3.00 (m, 1H); 2.40 (s, 3H); 1.60 (m, 3H); 1.40 (m, 1H); 1.18 (d, 3H); 1.12 (d, 3H).

IR (KBr, cm⁻¹) 3400-3500 (br), 2950, 2900, 1725, 1650, 1540, 1450, 1240, 1210, 1000, 760, 675, 580, 540.

MS ((+) ESI, m/z (%)) 706 (100 [M+H]⁺).

Anal. Calcd. for C₃₆H₄₃N₅O₈S.0.35 EtOAc: C, 60.98; H, 6.27; N, 9.51. Found: C, 50.31; H, 6.16; N, 9.33.

### EXAMPLE 271

### Synthesis of N-(Toluene-4-sulfonyl)-L-prolyl-L-4-(4-phenylcarbamylpiperazin-1-ylcarbonyloxy)phenylalanine

The title compound was prepared from the product of Example 270 using the procedure described in Method 7.

NMR data was as follows:

¹H NMR (DMSO-d₆, 400 MHz): δ 12.8 (s, 1H); 8.62 (s, 1H); 8.11 (d, 1H); 7.73 (d, 2H), 7.45 (m, 4H); 7.26 (m, 3H); 7.04 (m, 2H); 6.95 (m, 1H); 6.25 (d, 1H); 4.50 (m, 1H); 4.11 (m, 1H); 3.6 (br, 4H); 3.4 (br, 4H); 3.10 (m, 2H); 3.00 (m, 1H); 2.40 (s, 3H); 1.60 (m, 3H); 1.40 (m, 1H).

IR (KBr, cm⁻¹) 3400, 1725, 1650, 1540, 1450, 1240, 1210, 1000, 760, 675, 580, 540.

MS ((−) ESI, m/z (%)) 662 (100 [M−H]⁺).

### EXAMPLE 272

### Synthesis of N-(1-n-Butylpyrazole-4-sulfonyl)-L-(5,5-dimethyl)thiaprolyl-L-4-(N,N-dimethylcarbamyloxy)phenylalanine Isopropyl Ester

The title compound was prepared following the procedure described for the preparation of Example 137 and substitution of appropriate starting materials.

NMR data was as follows:

¹H NMR (CDCl₃): δ 7.89 (s, 1H), 7.83 (s, 1H), 7.21 (d, 2H), 7.06 (d, 1H), 7.02 (d, 2H), 5.04 (sept, 1H), 4.89-4.82 (m, 1H), 4.57 (d, 1H), 4.41 (d, 1H), 4.16 (t, 2H), 3.78 (s, 1H), 3.14 (dd, 1H), 3.06 (dd, 1H), 3.09 (s, 3H), 3.00 (s, 3H), 1.85 (pent, 2H), 1.36-1.23 (m, 2H), 1.27 (s, 3H), 1.24 (d, 3H), 1.21 (d, 3H), 1.16 (s, 3H).

¹³C NMR (CDCl₃): δ 170.4, 168.3, 154.9, 150.7, 139.2, 131.8, 130.3, 121.8, 117.0, 73.8, 69.6, 54.8, 53.2, 52.7, 50.6, 37.7, 36.6, 36.3, 31.8, 28.9, 24.0, 21.6, 21.5, 19.4, 13.3.

### EXAMPLE 273

### Synthesis of N-(Toluene-4-sulfonyl)-L-prolyl-L-4-(pyridin-4-ylcarbonyl)piperazin-1-ylcarbonyloxy)phenylalanine Isopropyl Ester

The title compound was prepared following the procedure described for the preparation of Example 4 and substitution of appropriate starting materials.

NMR data was as follows:

¹H NMR (DMSO-d₆, 400 MHz): δ 8.69 (dd, 2H), 8.28 (d, 1H); 7.71 (d, 2H); 7.43 (m, 4H); 7.26 (d, 2H); 7.04 (d, 2H); 4.86 (m, 1H); 4.42 (m, 1H); 4.05 (m, 1H); 3.4-3.8 (brm, 9H); 3.05 (m, 3H); 2.40 (s, 3H); 1.60 (m, 3H); 1.40 (m, 1H); 1.18 (d, 3H); 1.15 (d, 3H).

IR (KBr, cm⁻¹) 3400, 1725, 1650, 1510, 1200, 1160, 1100, 1010, 650, 600, 550.

MS ((+) ESI, m/z (%)) 692 (100 [M+H]⁺).

Anal. Calcd. for C₃₅H₄₁N₅O₉S.0.75H₂O: C, 59.60; H, 6.07; N, 9.93 Found: C, 59.45; H, 5.86; N, 9.88.

### EXAMPLE 274

### Synthesis of N-(Toluene-4-sulfonyl)-L-4-oxoprolyl-L-4-(N,N-dimethylcarbamyloxy)phenylalanine

The title compound was prepared from the product of Example 164 using the procedure described in Method 11.

Physical data was as follows:

MS [(−) ESI] [M−H]) 516.

### EXAMPLE 275

### Synthesis of N-(Toluene-4-sulfonyl)-L-trans-4-hydroxyprolyl-L-4-(N,N-dimethylcarbamyloxy)phenylalanine

The title compound was prepared from the product of Example 165 using the procedure described in Method 11.

Physical data was as follows:

MS [(−) ESI] [M−H]) 518.

### EXAMPLE 276

### Synthesis of N-(4—Cyanobenzenesulfonyl)-L-prolyl-L-4-(N,N-dimethylcarbamyloxy)phenylalanine Isopropyl Ester

The title compound was prepared following the procedure described for the preparation of Example 2 and substitution of appropriate starting materials, mp. 166-167° C.

### EXAMPLE 277

### Synthesis of N-(4-Aminobenzenesulfonyl)-L-prolyl-L-4-(N,N-dimethylcarbamyloxy)phenylalanine

The title compound was prepared from the product of Example 107 using the procedure described in Method 11.

Physical data was as follows:

Anal. Calcd. for C₂₃H₂₈N₄O₇S: C, 47.34; H, 4.84; N, 9.60. Found: C, 47.57; H, 5.20; N, 8.75.

### EXAMPLE 278

### Synthesis of N-(Toluene-4-sulfonyl)-L-4-oxoprolyl-L-4-(4-methylpiperazin-1-ylcarbonyloxy)phenylalanine tert-Butyl Ester

Acetonitrile (3 mL) was cooled to −40° C. (CH₃CN/dry ice). Oxalyl chloride (0.10 mL) was added. N-(Toluene-4-sulfonyl)-L-4-hydroxyprolyl-L-4-(4-methylpiperazin-1-ylcarbonyloxy)phenylalanine tert-butyl ester (300 mg) and dry DMSO (0.008 mL) were dissolved in acetonitrile (4 mL) and were added to the above solution. The reaction was stirred at −40° C. for half an hour under dry conditions. Triethylamine (0.33 mL) was added to the solution. The dry ice bath was removed after 5 minutes. The reaction was stirred at room temperature for 1 hour. The solvent was evaporated in vacuo, and ethyl acetate (15 mL) was added. The mixture was washed with water (3×), then washed with brine. The solution was dried over MgSO₄. The solvent was evaporated in vacuo, and the residue was flushed on a silica gel column to give 150 mg of the title compound, mp. 84-85° C.

### EXAMPLE 279

### Synthesis of N-(Toluene-4-sulfonyl)-L-prolyl-L-4-[3-(hydroxymethyl)piperidin-1-ylcarbonyloxy]phenylalanine

The title compound was prepared following the procedure described for the preparation of Example 4 and substitution of appropriate starting materials, mp. 84-85° C.

### EXAMPLE 280

### Synthesis of N-(Toluene-4-sulfonyl)-L-(4,4-difluoro)prolyl-L-4-(N,N-dimethylcarbamyloxy)phenylalanine Isopropyl Ester

The title compound was prepared following the procedure described for the preparation of Example 2 and substitution of appropriate starting materials.

Physical data was as follows:

MS: [(+) ESI], [M+NH]⁺ 599.

### EXAMPLE 281

### Synthesis of N-(Toluene-4-sulfonyl)-L-(4,4-difluoro)prolyl-L-4-(N,N-dimethylcarbamyloxy)phenylalanine

The title compound was prepared from the product of Example 280 using the procedure described in Method 7.

Physical data was as follows:

MS: [(+) ESI], [M+NH₄] 557.

### EXAMPLE 282

### Synthesis of N-(Toluene-4-sulfonyl)-L-prolyl-L-(4-benzoylpiperazin-1-ylcarbonyloxy)phenylalanine Isopropyl Ester

The title compound was prepared following the procedure described for the preparation of Example 4 and substitution of appropriate starting materials.

NMR data was as follows:

¹H NMR (DMSO-d₆, 400 MHz): δ 8.27 (d, 1H); 7.69 (d, 2H); 7.45 (m, 7H); 7.24 9d, 2H); 7.02 (d, 2H); 4.86 (m, 1H); 4.42 (m, 1H); 4.07 (m, 1H); 3.65 (br s, 4H); 3.45 (br s, 4H); 3.35 (m, 1H); 3.05 (m, 3H); 2.38 (s, 3H); 1.60 (m, 3H); 1.40 (m, 1H); 1.18 (d, 3H); 1.11 (d, 3H).

IR (KBr, cm⁻¹) 3400, 1725, 1675, 1625, 1510, 1425, 1350, 1250, 1175, 1110, 1010, 700, 660, 590, 550.

MS ((+) ESI, m/z (%)) 708 (100 [M+NH₂]⁺).

Anal. Calcd. for C₃₆H₄₂N₄O₈S.0.5H₂O: C, 61.79; H, 6.19; N, 8.01. Found: C, 61.64; H, 6.10; N, 7.72.

### EXAMPLE 283

### Synthesis of N-(1-Methyl-1H-imidazole-4-sulfonyl)-L-prolyl-L-4-(N,N-dimethylcarbamyloxy)phenylalanine Isopropyl Ester

The carbamate was prepared by treatment of 1-methylimidazole-4-sulfonyl-Pro-Try-iPr ester with 4-nitrophenyl chloroformate, followed by addition of dimethylamine (triethylamine, methylene chloride, 0° C., stirred at room temperature overnight.) The crude product was purified by flash chromatography (silica, 95:3:2 EtOAc:EtOH; Et₃N), followed by recrystallization from EtOAc. A white solid was obtained, mp 162-164° C. (8.7 g, 66%).

Physical data was as follows:

Anal. Calcd. for C₂₄H₃₃N₅O₇S: C, 53.82; H, 6.21; N, 13.08. Found: C, 53.47; H, 6.13; N, 12.96.

### EXAMPLE 284

### Synthesis of N-(Toluene-4-sulfonyl)-L-4-(thiomorpholin-4-ylcarbonyloxy)prolyl-L-4-(thiomorpholin-4-ylcarbonyloxy)phenylalanine

The title compound was prepared from the tert-butyl ester using the procedure described in Method 11, mp. 116-118° C.

### EXAMPLE 285

### Synthesis of N-(4—Cyanobenzenesulfonyl)-L-prolyl-L-4-(thiomorpholin-4-ylcarbonyloxy)phenylalanine Isopropyl Ester

The title compound was prepared following the procedure described for the preparation of Example 4 and substitution of appropriate starting materials, mp. 70-71° C.

### EXAMPLE 286

### Synthesis of N-(4-Amidinobenzenesulfonyl)-L-prolyl-L-4-(N,N-dimethylcarbamyloxy)phenylalanine Methyl Ester

Methanol (dry) was cooled to 0° C. HCl was bubbled in the solution for 15 minutes to make a saturated solution. Example 276 was added and the reaction mixture was stirred at 0° C. for 30 minutes then at room temperature for 24 hours. The solvent was evaporated. NH₃ in methanol (2M, 5 mL) was added. The reaction was stirred at room temperature for 24 hours. The solvent was evaporated. The residue was purified by reverse phase HPLC in CH₃CN:H₂O (20:80). At a retention time of 12.45 minutes, the product was isolated and freeze-dried to provide the title compound.

NMR data was as follows:

¹H NMR (in DMSO) multiplet at 1.47-1.55 ppm (1H), 1.63-1.72 ppm (3H's), singlet at 2.87 ppm (3H's), singlet at 3.02 ppm (3H's), multiplet at 3.05-3.10 ppm (2H's), multiplet at 3.17-3.22 ppm (1H), multiplet at 3.37-3.42 ppm (1H), singlet at 3.62 ppm (3H's), multiplet at 4.21-4.23 ppm (1H), quartet at 4.48-4.56 ppm (1H), doublet at 7.00-7.03 ppm (2H's), doublet at 7.23-7.26 ppm (2H's), a broad peak at 7.20-7.50 ppm, doublet at 8.02-8.03 ppm (4H's), doublet at 8.48-8.52 ppm (1H).

### EXAMPLE 287

### Synthesis of N-(Toluene-4-sulfonyl)-L-4-hydroxyprolyl-L-4-(thiomorpholin-4-ylcarbonyloxy)phenylalanine tert-Butyl Ester

The title compound was prepared following the procedure described for the preparation of Example 4 and substitution of appropriate starting materials, mp. 80-82° C.

### EXAMPLE 288

### Synthesis of N-(Toluene-4-sulfonyl)-L-4-hydroxyprolyl-L-4-(thiomorpholin-4-ylcarbonyloxy)phenylalanine

N-(Toluene-4-sulfonyl)-L-4-hydroxyprolyl-L-4-(thiomorpholin-4-ylcarbonyloxy)phenylalanine tert-butyl ester (160 mg) was dissolved in formic acid (7 mL). The reaction was stirred at room temperature for 6 hours. The solvent was evaporated and the residue purified using reverse phase HPLC in 20:80 CH₃CN/water. At a retention time of 5.85 minutes, 50 mg of the title compound was obtained, mp. 170-172° C.

### EXAMPLE 289

### Synthesis of N-(Toluene-4-sulfonyl)-L-prolyl-L-(4-benzoylpiperazin-1-ylcarbonyloxy)phenylalanine

The title compound was prepared from the product of Example 282 using the procedure described in Method 1.

NMR data was as follows:

¹H NMR (DMSO-d₆, 400 MHz): δ 12.8 (s, 1H); 8.27 (d, 1H); 7.69 (d, 2H); 7.45 (m, 7H); 7.24 (d, 2H); 7.02 (d, 2H); 4.42 (m, 1H); 4.07 (m, 1H); 3.65 (br s, 4H); 3.45 (br s, 4H); 3.35 (m, 1H); 3.05 (m, 3H); 2.38 (s, 3H); 1.60 (m, 3H); 1.40 (m, 1H).

IR (KBr, cm⁻¹) 3400, 1725, 1675, 1625, 1510, 1425, 1350, 1260, 1175, 1110, 1010, 700, 660, 590, 550.

MS ((+) ESI, m/z (%)) 666 (100 [M+NH₄]⁺).

Anal. Calcd. for C₃₃H₃₆N₄O₈S.0.66H₂O: C, 60.00; H, 5.69; N, 8.48 Found: C, 60.36; H, 5.70; N, 7.81.

### EXAMPLE 290

### Synthesis of N-(4-Amidinobenzenesulfonyl)-L-prolyl-L-4-(thiomorpholin-4-ylcarbonyloxy)phenylalanine Methyl Ester

The title compound was prepared following the procedure described for the preparation of Examples 285 and 286.

Physical data was as follows:

MS: [(+) ESI] [M+H] 604.

### EXAMPLE 291

### Synthesis of N-(3-Fluorobenzenesulfonyl)-L-prolyl-L-4-(N,N-dimethylcarbonyloxy)phenylalanine

The title compound was prepared from the product of Example 166 using the procedure described in Method 11, mp. 82-83° C.

### EXAMPLE 292

### Synthesis of N-(Toluene-4-sulfonyl)-L-prolyl-L-4-[N-methyl-N-(2-(N′-methyl-N′-toluenesulfonyl-amino)ethyl)carbamyloxy]phenylalanine Isopropyl Ester

The title compound was prepared following the procedure described for the preparation of Example 4 and substitution of appropriate starting materials.

NMR data was as follows:

¹H NMR (DMSO-d₆, 400 MHz): δ 8.27 (d, 1H); 7.71 (d, 2H); 7.69 (d, 2H); 7.40 (m, 4H); 7.24 (d, 2H); 6.99 (d, 2H); 4.86 (m, 1H); 4.43 (m, 1H); 4.06 (m, 1H); 3.51 (m, 1H); 3.2-3.35 (m, 3H); 2.9-3.2 (overlapping m, 7H); 2.67 (d, 3H); 2.38 (s, 6H); 1.60 (m, 3H); 1.40 (m, 1H); 1.20 (d, 3H); 1.15 (d, 3H).

IR (KBr, cm⁻) 3400, 2975, 2950, 1725, 1680, 1510, 1450, 1400, 1280, 1225, 1150, 1110, 800, 730, 675, 575, 550.

MS ((+) ESI, m/z (%)) 760 (100 [M+NH₄]⁺).

Anal. Calcd. for C₃₆H₄₆N₄O₉S₂: C, 58.20; H, 6.24; N, 7.54. Found: C, 57.90; H, 6.30; N, 7.34.

### EXAMPLE 293

### Synthesis of N-(Toluene-4-sulfonyl)-L-prolyl-L-4-[N-(2-(N′-phenylaminocarbonyloxy)ethyl)carbamyloxy)]phenylalanine Isopropyl Ester

The title compound was prepared following the procedure described for the preparation of Example 4 and substitution of appropriate starting materials.

NMR data was as follows:

¹H NMR (DMSO-d₆, 400 MHz): δ 9.67 (s, 1H); 8.27 (d, 1H); 7.72 (d, 2H); 7.47 (d, 2H); 7.42 (d, 2H); 7.24 (m, 4H); 6.98 (m, 3H); 4.87 (m, 1h); 4.45 (m, 1H); 4.18 (m, 2H); 4.05 (m, 1H); 3.4 (m, 3H); 3.05 (m, 3H) 2.40 (s, 3H); 1.6 (m, 3H); 1.40 (m, 1H); 1.2 (d, 3H); 1.15 (d, 3H).

IR (KBr, cm⁻¹) 3350, 2950, 1725, 1675, 1600, 1550, 1500, 1325, 1200, 1150, 1100, 650, 575, 525.

MS ((+) ESI, m/z (%)) 698 (100 [M+NH₄]⁺).

Anal. Calcd. for C₃₄H₄₀N₄O₉S.0.21 EtOAc. 0.5H₂O: C, 59.08; H, 6.07; N, 7.91. Found: C, 59.08; H, 6.02; N, 7.80.

### EXAMPLE 294

### Synthesis of N-(4-Fluorobenzenesulfonyl)-L-4-(trans-hydroxy)prolyl-L-4-(N,N-dimethylcarbamyloxy)phenylalanine Isopropyl Ester

The title compound was prepared following the procedure described in Example 2 and substitution of appropriate starting materials.

Physical data was as follows:

MS: [(+) ESI], [M+NH₄] 583.

### EXAMPLE 295

### Synthesis of N-(4-Fluorobenzenesulfonyl)-L-4-(trans-hydroxy)prolyl-L-4-(N,N-dimethylcarbamyloxy)phenylalanine tert-Butyl Ester

The title compound was prepared following the procedure described in Example 2 and substitution of appropriate starting materials.

Physical data was as follows:

MS: [(+) ESI], [M+NH₄] 597.

### EXAMPLE 296

### Synthesis of N-(4-Amidinobenzenesulfonyl)-L-prolyl-L-4-(N,N-dimethylcarbamyloxy)phenylalanine

The title compound was prepared from the product of Example 286 using the procedure described in Method 5, mp. 130-132° C.

### EXAMPLE 297

### Synthesis of piperazine-1,4-dicarboxylic Acid Bis-{4-[(2S)-2-tert-butoxycarbonyl-2-((4R)-5,5-dimethyl-3-(toluene-4-sulfonyl)thiazolidine-4-carboxamido)ethyl]phenyl}Ester

The title compound was prepared following the procedure described in Example 4, except that 0.5 equivalents of piperazine were used.

Physical data was as follows:

Anal. Calcd. for C₅₈H₇₄N₆O₁₄S₄: C, 57.69; H, 6.18; N, 6.96. Found: C, 58.01; H, 6.07; N, 6.68.

### EXAMPLE 298

### Synthesis of piperazine-1,4-dicarboxylic Acid Bis-{4-[(2S)-2-carboxy-2-((4R)-5,5-dimethyl-3-(toluene-4-sulfonyl)thiazolidine-4-carboxamido)ethyl]phenyl} Ester

The title compound was prepared by hydrolysis of the di-t-butyl ester from Example 297 with formic acid to give a white foam (300 mg, quantitative).

Physical data was as follows:

Anal. Calcd. for C₅₀H₅₈N₆O₁₄S₄: C, 54.83; H, 5.34; N, 7.67 Found: C, 55.10; H, 5.57; N, 7.37.

Other compounds of formulae I and Ia prepared by the methods described above include those set forth in Examples 299-368 in Table 13 below.

TABLE 13

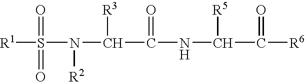

| Ex. | | | | | |
|---|---|---|---|---|---|
| No. | R¹ | R² | R³ | R⁵ | R⁶ |
| 299 | p-CH₃-φ- | R²/R³ = cyclic | | p-[(CH₃)₂NC(O)O-]benzyl- | —OC(CH₃)₃ |
| | | —CH₂CH₂—NH—CH₂— | | | |
| | | (L-piperazinyl) | | | |
| 300 | p-F-φ- | R²/R³ = cyclic | | p-[(2-(hydroxymethyl)pyrrolidin-1-yl-C(O)O-]- | —OC(CH₃)₃ |
| | | —CH₂—S—C(CH₃)₂— | | benzyl- | |
| | | (L-5,5- | | | |
| | | dimethylthiazolidin- | | | |
| | | 4-yl | | | |
| 301 | p-F-φ- | R²/R³ = cyclic | | p-[(2-(hydroxymethyl)pyrrolidin-1-yl-C(O)O-]- | —OH |
| | | —CH₂—S—C(CH₃)₂— | | benzyl- | |
| | | (L-5,5- | | | |
| | | dimethylthiazolidin | | | |
| 4-yl) | | | | | |
| 302 | p-CH₃-φ- | R²/R³ = cyclic | | p-[(2-(CH₃OC(O)-)pyrrolidin-1-yl)-C(O)O-] | —OC(CH₃)₃ |
| | | 3 carbon atoms | | benzyl | |
| | | (L-pyrrolidinyl) | | | |
| 303 | p-F-φ- | R²/R³ = cyclic | | 3-chloro-4-[(thiomorphohn-4-yl)-C(O)O-] | —OH |
| | | —CH₂—S—C(CH₃)₂— | | benzyl | |
| | | (L-5,5- | | | |
| | | dimethyithiazolidin | | | |
| | | 4-yl) | | | |
| 304 | p-F-φ- | R²/R³cyclic | | p-[(4-(pyridin-2-yl)piperazin-1-yl)-C(O)O-] | —OH |
| | | —CH₂—S-C(CH₃)₂— | | benzyl- | |
| | | (L-5,5- | | | |
| | | dimethylthiazolidin- | | | |
| | | 4-yl | | | |
| 305 | p-F-φ- | R²/R³ = cyclic | | p-[(4-(pyridin-2-yl)piperazin-1-yl)-C(O)O]- | —OC(CH₃)₃ |
| | | —CH₂—S—C(CH₃)₂— | | benzyl- | |
| | | (L-5,5- | | | |
| | | dimethyithiazolidin- | | | |
| | | 4-yl) | | | |
| 306 | p-CH3-φ- | R²/r³ = cyclic | | p-[(thiomorpholin-4-yl)-C(O)O-]benzyl- | —OC(CH₃)₃ |
| | | —CH₂CH(OH)CH₂— | | | |
| | | (L-4- | | | |
| | | hydroxypyrrolidinyl) | | | |
| 307 | p-CH3-φ- | R²R³ = cyclic | | p-[(CH₃)₂NC(O)O-]benzyl- | —O(CH₂CH₂O)₂CH₃ |
| | | 3 carbon atoms | | | |
| | | (L-pyrrolidinyl) | | | |
| 308 | p-F-φ- | R²/R³ = cyclic | | p-[(4-(pyrimidin-2-yl)piperazin-1-yl)- | —OC(CH₃)₃ |
| | | —CH₂—S—C(CH₃)₂— | | C(O)O-]benzyl | |
| | | (L-5,5- | | | |
| | | dimethyithiazolidin | | | |
| | | 4-yl) | | | |
| 309 | p-F-φ- | R²/R³ = cyclic | | 3-fluoro-4-[(CH₃)₂NC(O)O-]-benzyl- | —OCH(CH₃)₂ |
| | | —CH₂—S—C(CH₃)₂— | | | |
| | | (L-5,5- | | | |
| | | dimethyithiazolidin- | | | |
| | | 4-yl) | | | |
| 310 | p-CH₃-φ- | R²/R³ = cyclic | | p-[(CH₃)₂NC(O)O-]benzyl- | —OC(CH₃)₃ |
| | | —CH₂—CH₂N— | | | |
| | | (—SO₂CH₃)—CH₂— | | | |
| | | (L-4- | | | |
| | | methanesulfonyl | | | |
| | | piperazinyl) | | | |
| 311 | R¹/R^{2 =} | | H | p-[(CH₃)₂NC(O)O-]benzyl- | —OH |
| | 1,1-dioxo-2,3-dihydro- | | | | |
| | 3,3-dimethyl- | | | | |
| | 1,2-benzisothiazol-2-yl | | | | |
| 312 | R¹/R² = | | H | p-[(CH₃)₂NC(O)O-]benzyl- | —OC(CH₃)₃ |
| | N-2,10- | | | | |
| | camphorsultamyl- | | | | |
| 313 | R¹/R² = | | H | p-[(CH₃)₂NC(O)O-]benzyl- | —OH |
| | N-2,10- | | | | |
| | camphorsultamyl- | | | | |
| 314 | R¹/R² = | | H | 3-chloro-4-[(CH₃)₂NC(O)O-]-benzyl- | —OCH(CH₃)₂ |
| | N-2,10- | | | | |
| | camphorsultamyl- | | | | |
| 315 | p-Brφ- | R²/R³ = cyclic | | p-[(CH₃)₂NC(O)O-]benzyl- | —OC(CH₃)₃ |
| | | —CH₂—S—C(CH₃)₂— | | | |
| | | (L-5,5- | | | |
| | | dimethyithiazolidin- | | | |
| | | 4-yl | | | |
| 316 | p-Br-φ- | R²/R³ = cyclic | | p-[(CH₃)₂NC(O)O-]benzyl- | —OH |
| | | —CH₂—S—C(CH₃)₂— | | | |
| | | (L-5,5- | | | |
| | | dimethyithiazolidin- | | | |
| | | 4-yl | | | |
| 317 | p-CH₃-φ- | R² /R³ = cyclic | | p-[(4-methylpiperazin-1-yl)-C(O)O-]benzyl- | —OH |
| | | —CH₂—CH(OH)— | | | |
| | | CH₂— | | | |
| | | (L-4- | | | |
| | | hydroxypyrrolidinyl) | | | |
| 318 | p-F-φ- | R²/R³ = cyclic | | p-[(4-pyrimidin-2-yl)piperazin-1-yl)-C(O)O-] | —OH |
| | | —CH₂—S—C(CH₃)₂— | | benzyl | |
| | | (L-5,5- | | | |
| | | dimethylthiazolidin-4-yl) | | | |
| 319 | p-F-φ- | R²/R³ = cyclic | | p-[4-(pyridin-2-yl)piperazin-1-yl)C(O)O-] | —OCH(CH₃)₂ |
| | | —CH₂—S—C(CH₃)₂— | | benzyl | |
| | | (L-5,5- | | | |
| | | dimethylthiazolidin | | | |
| | 4-yl) | | | | |
| 320 | p-F-φ- | R²/R³ = cyclic | | p-[(CH₃)₂NC(O)O-]benzyl- | —OH |
| | | -CH2-CH2-S- | | | |
| | | (thiazolidin-2-yl) | | | |
| 321 | p-F-φ- | R²/R³ = cyclic | | p-[(CH₃)₂NC(O)O-]benzyl- | —OC(CH₃)₃ |
| | | —CH₂—CH₂—S— | | | |
| | | (thiazolidin-2-yl) | | | |
| 322 | p-CH₃-φ- | R²/R³ =cyclic | | p-[(thiomorpholin-4-yl)C(O)O-]benzyl- | —OH |
| | | —CH₂—C(O)—CH₂— | | | |
| | | (L-4- | | | |
| | | (oxypyrrolidinyl) | | | |
| 323 | p-CH₃-φ- | R²/R³ = cyclic | | p-[(4-methylpiperazin-1-yl)-C(O)O-]benzyl- | —OH |
| | | —CH₂—C(O)—CH₂— | | | |
| | | (L-4- | | | |
| | | oxypyrrolidinyl) | | | |
| 324 | p-F-φ- | R²/R³ = cyclic | | p-[(4-(pyridin-2-yl)piperazin-1-yl)- | —OH |
| | | —CH₂—CH₂—S— | | C(O)O-]benzyl- | |
| | | (thiazolidin-2-yl) | | | |
| 325 | p-NO₂-φ- | R²/R³ = cyclic | | p-[(4-(pyridin-2-yl)piperazin-1-yl)- | —OC(CH₃)₃ |
| | | 3 carbon atoms | | C(O)O-]benzyl | |
| | | (L-pyrrolidinyl) | | | |
| 326 | p-F-φ- | R²/R³ = cyclic | | p-[(4-(pyridin-2-yl)piperazin-1-yl)- | —OC(CH₃)₃ |
| | | —CH₂—CH₂—S— | | C(O)O-]benzyl | |
| | | (thiazolidin-2-yl) | | | |
| 327 | p-Br-φ- | R²/R³ = cyclic | | p-[(4-(pyridin-2-yl)piperazin-1-yl)- | —OH |
| | | —CH₂—S—C(CH₃)₂— | | C(O)O-]benzyl- | |
| | | (L-5,5- | | | |
| | | dimethyithiazolidin- | | | |
| | | 4-yl) | | | |
| 328 | p-CH₃-φ- | R²/R³ = cyclic | | p-[(4-(φNHC(S)-)piperazin-1-yl)-C(O)O-] | —OCH(CH₃)₂ |
| | | 3 carbon atoms | | benzyl- | |
| | | (L-pyrrolidinyl) | | | |
| 329 | p-F-φ- | R² /R³ = cyclic | | p-[(4-CH₃-homopiperazin-1-yl)-C(O)O-]benzyl- | —OC(CH₃)₃ |
| | | —CH₂—CH₂—S— | | | |
| | | (thiazolidin-2-yl) | | | |
| 330 | p-CH₃-φ- | R²/R³ = cyclic | | p-[(CH₃)₂NC(O)O-]benzyl- | —OC(CH₃)₃ |
| | | —CH₂CH(—OSO₂CH₃)- | | | |
| | | CH₂-(L-4- | | | |
| | | methanesulfoxy | | | |
| | | (pyrrolidinyl) | | | |
| 331 | p- | R²/R³ = cyclic | | p-[(CH₃)₂NC(O)O-]benzyl- | —OH |
| | H₂NC(O) | 3 carbon atoms | | | |
| | -φ- | (L-pyrrolidinyl) | | | |
| 332 | p- | R²/R³ = cyclic | | p-[(thiomorpholin-4-yl)C(O)O-]benzyl- | —OH |
| | H₂NC(O) | 3 carbon atoms | | | |
| | -φ- | (L-pyrrolidinyl) | | | |
| 333 | p- | R²/R³ = cyclic | | p-[(thiomorpholin-4-yl)C(O)O-]benzyl- | —OH |
| | H₂NC(═N) | 3 carbon atoms | | | |
| | -φ- | (L-pyrrolidinyl) | | | |
| 334 | p-NO₂-φ- | R²/R³ = cyclic | | p-[(4-(pyridin-2-yl)piperazin-1-yl)-C(O)O-] | —OH |
| | | 3 carbon atoms | | benzyl | |
| | | (L-pyrrolidinyl) | | | |
| 335 | p-F-φ- | R²/R³ = cyclic | | 3-chloro-4-[(4-pyridin-2-yl)piperazin-1- | —OCH₂CH₃ |
| | | —CH₂—S—C(CH₃)₂— | | yl)C(O)O-]- | |
| | | (L-5,5- | | benzyl- | |
| | | dimethylthiazolidin- | | | |
| | | 4-yl) | | | |
| 336 | p-F-φ- | R²/R³ = cyclic | | 3-chloro-4-[(4-pyridin-2-yl)piperazin-1- | —OH |
| | | —CH₂—S—C(CH₃)₂— | | yl)C(O)O-]- | |
| | | (L-5,5- | | benzyl- | |
| | | dimethylthiazolidin- | | | |
| | | 4-yl) | | | |
| 337 | p-F-φ- | R²/R³ = cyclic | | p-[(4-CH₃-homopiperazin-1-yl-C(O)O-]benzyl- | —OH |
| | | —CH₂—CH₂—S— | | | |
| | | (thiazolidin-2-yl) | | | |
| 338 | 1- | R²/R³ = cyclic | | 3-chloro-4-[(CH₃)₂NC(O)O-]benzyl- | —OCH(CH₃)₂ |
| | methylpyrazol- | —CH₂—S—C(CH₃)₂— | | | |
| | 4-yl- | (L-5,5- | | | |
| | | dimethylthiazolidin- | | | |
| | | 4-yl) | | | |
| 339 | 1- | R²/R³ = cyclic | | p-[(4-(pyridin-2-yl)piperazin-1-yl)C(O)O-] | —OCH(CH₃)₂ |
| | methylimidazol-4-yl- | 3 carbon atoms | | benzyl- | |
| | | (L-pyrrolidinyl) | | | |
| 340 | 1- | R²/R³ = cyclic | | p-[(4-(pyridin-2-yl)piperazin-1-yl)C(O)O-] | —OC(CH₃)₃ |
| | methylimidazol- | 3 carbon atoms | | benzyl- | |
| | 4-yl- | (L-pyrrolidinyl) | | | |
| 341 | p-CH₃-φ- | R²/R³ = cyclic | | p-[(4-(pyridin-2-yl)piperazin-1-yl)C(O)O-] | —OH |
| | | 3 carbon atoms | | benzyl | |
| | | (L-pyrrolidinyl) | | | |
| 342 | p-CH₃-φ- | R²/R³ = cyclic | | p-[(4-(pyridin-2-yl)piperazin-1-yl)C(O)O-] | —OC(CH₃)₃ |
| | | 3 carbon atoms | | benzyl- | |
| | | (L-pyrrolidinyl) | | | |
| 343 | p-CH₃-φ- | R²/R³ = cyclic | | p-[(4-(pyridin-2-yl)piperazin-1-yl)-C(O)O-] | —OCH(CH₃)₂ |
| | | 3 carbon atoms | | benzyl- | |
| | | (L-pyrrolidinyl) | | | |
| 344 | p-F-φ- | R²/R³ = cyclic | | p-[(4-(pyridin-2-yl)piperazin-1-yl)C(O)O-] | —OCH(CH₃)₂ |
| | | 3 carbon atoms | | benzyl | |
| | | (L-pyrrolidinyl) | | | |
| 345 | p-F-φ- | R²/R³ = cyclic | | p-[(4-(pyridin-2-yl)piperazrn-1-yl)C(O)O-] | —OC(CH₃)₃ |
| | | 3 carbon atoms | | benzyl- | |
| | | (L-pyrrolidinyl) | | | |
| 346 | p-CH₃-φ- | R²/R³ = cyclic | | p-[(CH₃)₂NC(O)O-]benzyl- | —OH |
| | | —CH₂CH₂N(—SO₂— | | | |
| | | CH₃)CH₂— | | | |
| | | (4-methanesulfonyl- | | | |
| | | piperazin-2-yl) | | | |
| 347 | p-CH₃-φ- | R²/R³ = cyclic | | p-[(CH₃)₂NC(O)O-]benzyl- | —OH |
| | | —CH₂CH(—OSO₂— | | | |
| | | CH₃)CH₂— | | | |
| | | (L-4- | | | |
| | | methanesulfoxy- | | | |
| | | pyrrolidinyl) | | | |
| 348 | CH₃— | —CH₂φ | H | p-[(CH₃)₂NC(O)O-]benzyl- | —OC(CH₃)₃ |
| 349 | p-Br-φ- | R²/R³ = cyclic | | p-[(4-(pyridin-2-yl)piperazin-1-yl)C(O)O-] | —OC(CH₃)₃ |
| | | —CH₂—S—C(CH₃)₂— | | benzyl- | |
| | | (L-5,5- | | | |
| | | dimethylthiazolidin- | | | |
| | | 4-yl) | | | |
| 350 | p-CF₃O- | R²/R³ = cyclic | | p-[(CH₃)₂NC(O)O-]benzyl- | —OH |
| | φ- | —CH₂—S—C(CH₃)₂— | | | |
| | | (L-5,5- | | | |
| | | dimethylthiazolidin- | | | |
| | | 4-yl) | | | |
| 351 | p-CF₃O- | R²/R³ = cyclic | | p-[(CH₃)₂NC(O)O-]benzyl- | —OC(CH₃)₃ |
| | φ- | —CH₂—S—C(CH₃)₂— | | | |
| | | (L-5,5- | | | |
| | | dimethylthiazolidin- | | | |
| | | 4-yl) | | | |
| 352 | p-CF₃O- | R²/R³ = cyclic | | p-[(4-(pyridin-2-yl)piperazin-1-yl)C(O)O-] | —OC(CH₃)₃ |
| | φ- | —CH₂—S—C(CH₃)₂- | | benzyl- | |
| | | (L-5,5- | | | |
| | | dimethylthiazolidin-4-yl) | | | |
| 353 | p-F-φ- | R²/R³ = cyclic | | p-[(4-(pyridin-2-yl)piperazin-1-yl)C(O)O-] | —OH |
| | | 3 carbon atoms | | benzyl- | |
| | | (L-pyrrolidinyl) | | | |
| 354 | p-F-φ- | R²/R³ = cyclic | | p-[(4-(pyridin-2-yl)piperazin-1-yl)C(O)O-] | —OH |
| | | —CH₂CH(OH)CH₂— | | benzyl- | |
| | | (L-4- | | | |
| | | hydroxypyrrolidinyl) | | | |
| 355 | p-CF₃O- | R²/R³ = cyclic | | p-[(4-(pyridin-2-yl)piperazin-1-yl)C(O)O-] | —OH |
| | φ- | —CH₂—S—C(CH₃)₂- | | benzyl- | |
| | | (L-5,5- | | | |
| | | dimethylthiazolidin- | | | |
| | | 4-yl) | | | |
| 356 | 1- | R²/R³ = cyclic | | 3-chloro-4-[(CH₃)₂NC(O)O-]-benzyl- | —OH |
| | methylimidazol- | 3 carbon atoms | | | |
| | 4-yl- | (L-pyrrolidinyl) | | | |
| 357 | 1- | R²/R³ = cyclic | | 3-chloro-4-[(CH₃)₂NC(O)O-]-benzyl- | —OCH(CH₃)₂ |
| | methylimidazol- | 3 carbon atoms | | | |
| | 4-yl- | (L-pyrrolidinyl) | | | |
| 358 | 1- | R²/R³ = cyclic | | p-[(4-(pyridin-2-yl)piperazin-1-yl)-C(O)O-] | —OH |
| | methylimidazol- | 3 carbon atoms | | benzyl- | |
| | 4-yl- | (L-pyrrolidinyl) | | | |
| 359 | 1- | R²/R³ = cyclic | | p-[(4-(pyridin-2-yl)piperazin-1-yl)C(O)O-] | —OH |
| | methylimidazol- | —CH₂—S—C(CH₃)₂— | | benzyl- | |
| | 4-yl | (L-5,5- | | | |
| | | dimethyithiazolidin | | | |
| | | 4-yl) | | | |
| 360 | 1- | R²/R³ = cyclic | | p-[(4-(pyridin-2-yl)piperazin-1-yl)C(O)O-] | —OH |
| | methylpyrazol- | 3 carbon atoms | | benzyl | |
| | 4-yl- | | | | |
| 361 | 1- | R²/R³ = cyclic | | p-[(4-(pyridin-2-yl)piperazin-1-yl)C(O)O-] | —OCH(CH₃)₂ |
| | methylpyrazol- | 3 carbon atoms | | benzyl- | |
| | 4-yl- | (L-pyrrolidinyl) | | | |
| 362 | 1- | R²/R³ = cyclic | | p-[(4-(pyridin-2-yl)piperazin-1-yl)C(O)O-] | —OC(CH₃)₃ |
| | methylpyrazol- | 3 carbon atoms | | benzyl | |
| | 4-yl- | (L-pyrrolidinyl) | | | |
| 363 | 1- | R²/R³ = cyclic | | p-[(4-(pyridin-2-yl)piperazin-1-yl)C(O)O-] | —OC(CH₃)₃ |
| | methylpyrazol- | —CH₂—S—C(CH₃)₂— | | benzyl- | |
| | 4-yl- | (L-5,5- | | | |
| | | dimethylthiazolidin | | | |
| | | 4-yl | | | |
| 364 | 1- | R²/R³ = cyclic | | 3-chloro-4-[(4-(pyridin-2-yl)piperazin-1- | —OCH(CH₃)₂ |
| | methylpyrazol- | 3 carbon atoms | | yl)C(O)O-]benzyl- | |
| | 4-yl- | (L-pyrrolidinyl) | | | |
| 365 | 1- | R²/R³ = cyclic | | p-[(CH₃)₂NC(O)O-]benzyl- | —OCH₂CH₂Oφ |
| | methylpyrazol- | —CH₂—S—C(CH₃)₂— | | | |
| | 4-yl- | (L-5,5- | | | |
| | | dimethylthiazolidin- | | | |
| | | 4-yl) | | | |
| 366 | 1- | R²/R³ = cyclic | | 3-chloro-4-[(4-pyridin-2-yl)piperazin-1- | —OH |
| | methylpyrazol- | —CH₂—S—C(CH₃)₂— | | yl)C(O)O-]- | |
| | 4-yl- | (L-5,5- | | benzyl- | |
| | dimethylthiazolidin- | | | | |
| | | 4-yl) | | | |
| 367 | 1- | R²/R³ cyclic | | 3-chloro-4-[(4-pyridin-2-yl)piperazin-1- | —OCH₂CH₃ |
| | methylpyrazol- | —CH₂—S—C(CH₃)₂— | | yl)C(O)O-]- | |
| | 4-yl- | (L-5,5- | | benzyl- | |
| | | dimethylthiazolidin- | | | |
| | | 4-yl) | | | |
| 368 | 1,5- | R²/R³ = cyclic | | p-[4-[5-CF₃-pyridin-2-yl)piperazin-1 yl)-C(O)O-] | —OH |
| | dimethyl- | —CH₂—S—C(CH₃)₂— | | benzyl- | |
| | 3- | (L-5,5- | | | |
| | chloropyrazol- | dimethylthiazolidin- | | | |
| | 4-yl- | 4-yl) | | | |

In addition, Examples 317, 322, 323, 330, 331, 332, 333 and 347 in Table 13 are exemplified as follows:

### EXAMPLE 317

### Synthesis of N-(Toluene-4-sulfonyl)-L-(4-hydroxy)prolyl-L-4-(4-methylpiperazin-1-ylcarbonyloxy)phenylalanine

The starting N-(toluene-4-sulfonyl)-L-(4-hydroxy)prolyl-L-4-(4-methylpiperazin-1-ylcarbonyloxy)phenylalanine tert-butyl ester (300 mg) was dissolved in formic acid (15 mL). The reaction was stirred at room temperature for 72 hours. The solvent was evaporated and the residue was purified using HPLC, reverse phase, 20-80% CH₃CN/water. At a retention time of 10.75 minutes, 82 mg of the title compound was obtained, mp: 128-130° C.

### EXAMPLE 322

### Synthesis of N-(Toluene-4-sulfonyl)-L-(4-oxo)prolyl-L-4-(thiomorpholin-4-ylcarbonyloxy)phenylalanine

The starting N-(toluene-4-sulfonyl)-L-(4-oxo)prolyl-L-4-(thiomorpholin-4-ylcarbonyloxy)phenylalanine tert-butyl ester (130 mg) was dissolved in formic acid (7 mL). The reaction was stirred at room temperature for 6 hours. The solvent was evaporated in vacuo to give 150 mg of the desired product, mp: 1,1-112° C.

### EXAMPLE 323

### Synthesis of N-(Toluene-4-sulfonyl)-L-(4-oxo)prolyl-L-4-(4-methylpiperazin-1-ylcarbonyloxy)phenylalanine

The starting N-(toluene-4-sulfonyl)-L-(4-oxo)prolyl-L-4-(4-methylpiperazin-1-ylcarbonyloxy)phenylalanine tert-butyl ester (150 mg) was dissolved in formic acid (7 mL). The reaction was stirred at room temperature for 6 hours. The solvent was evaporated in vacuo, and the residue was purified using HPLC, reverse phase, 20-80% CH₃CH/water. The retention time was 10.34 minutes. The product was freeze dried to yield 82 mg of the title compound, mp: 99-101° C.

### EXAMPLE 330

### Synthesis of N-(Toluene-4-sulfonyl)-L-(4-methanesulfonyloxy)prolyl-L-4-(N,N-dimethylcarbamyloxy)phenylalanine tert-Butyl Ester

The starting N-(toluene-4-sulfonyl)-L-(4-hydroxy)prolyl-L-4-(N,N-dimethylcarbamyloxy)phenylalanine tert-butyl ester (300 mg) and methylsulfonyl chloride was dissolved in THF (7 mL) at 0° C. in an ice bath. Triethylamine (0.21 mL) was added. The ice bath was removed after 10 minutes. The reaction mixture was stirred at room temperature for 24 hours. Ethyl acetate (20 mL) as added. The mixture was washed with citric acid (5%, 20 mL, 2×), and washed with saturated NaHCO₃ solution (20 mL), then with brine. The solution was dried over MgSO₄. The solvent was evaporated, and the residue was flushed on a silica gel column. The solvent was evaporated in vacuo to give 300 mg of the desired product, mp: 73-74° C.

### EXAMPLE 331

### Synthesis of N-(4-Aminobenzenesulfonyl)-L-prolyl-L-4-(N,N-dimethylcarbamyloxy)phenylalanine

The starting N-(4-aminobenzenesulfonyl)-L-prolyl-L-4-(N,N-dimethylcarbamyloxy)phenylalanine methyl ester (300 mg) and LiOH solution (2M, 0.6 mL) were added to methanol (6 mL). The reaction was stirred at room temperature for 7 hours. The solvent was evaporated in vacuo, and the residue was purified using HPLC, reverse phase, 20-80% CH₃CN/water. At a retention time of 12.11 minutes, 27 mg of the desired product were obtained, mp: 130-132° C.

### EXAMPLE 332

### Synthesis of N-(4-Aminocarbonylbenzenesulfonyl)-L-prolyl-L-4-(thiomorpholin-4-ylcarbonyloxy)phenylalanine

The starting N-(4-aminocarbonylbenzenesulfonyl)-L-prolyl-L-4-(thiomorpholin-4-ylcarbonyloxy)phenylalanine methyl ester (300 mg) and LiOH solution (2M, 0.5 mL) were added to methanol (6 mL). The reaction was stirred at room temperature for 8 hours. The solvent was evaporated in vacuo, and the residue purified using HPLC, reverse phase, 20-80% CH₃CN/water. At a retention time of 12.69 minutes, 20 mg of the desired product was obtained, mp: 123-125° C.

### EXAMPLE 333

### Synthesis of N-(4-Amidinobenzenesulfonyl)-L-prolyl-L-4-(thiomorpholin-4-ylcarbonyloxy)phenylalanine

The starting N-(4-amidinobenzenesulfonyl)-L-prolyl-L-4-(thiomorpholin-4-ylcarbonyloxy)phenylalanine methyl ester (300 mg) and LiOH solution (2M, 0.5 mL) were added to methanol (6 mL). The reaction was stirred at room temperature for 8 hours. The solvent was evaporated in vacuo, and the residue was purified using HPLC, reverse phase, 20-80% CH₃CN/water. At a retention time of 11.78 minutes, 25 mg of the desired product were obtained, mp: 123-125° C.

### EXAMPLE 347

### Synthesis of N-(Toluene-4-sulfonyl)-L-(4-methanesulfonyloxy)prolyl-L-4-(N,N-dimethylcarbamyloxy)phenylalanine

The starting N-(toluene-4-sulfonyl)-L-(4-methanesulfonyloxy)prolyl-L-4-(N,N-dimethylcarbamyloxy)phenylalanine tert-butyl ester (200 mg) was dissolved in formic acid (5 mL). The reaction mixture was stirred at room temperature for 6 hours. The solvent was evaporated in vacuo to provide the desired product (195 mg), mp: 83-84° C.

### EXAMPLE 369

### Synthesis of N-(Toluene-4-sulfonyl)-L-prolyl-4-(α-methylbenzyloxy)-L-phenylalanine

N-(Toluene-4-sulfonyl)-L-prolyl-L-tyrosine methyl ester (785 mg, 1.89 mmol) was dissolved in DMF (20 μL) at room temperature. To this was added K₂CO₃ (1.1 eq, 281 mg) and 1-bromoethyl benzene (1.1 eq, 284 μL). The reaction was stirred for 12 hours at room temperature. Ethyl acetate (100 mL) was added, and the organic layer washed several times with brine (5×50 mL). The organic layer was dried over MgSO₄. Upon filtration and evaporation of the solvents under reduced pressure, an oil was isolated. The crude material was purified by elution on silica gel (EtOAc/hexanes (1:4)). The desired material was isolated in 32% yield (330 mg, 0.6 mmol). The methyl ester (330 mg, 0.6 mmol) was then converted to the corresponding acid upon treatment with NaOH (1.1 eq, 27 mg), in MeOH:H₂O (1:1) (15 mL), for 4 hours at room temperature. EtOAc was added as well as water. The aqueous layer was collected and acidified with 1N HCl to pH 2.5, and reextracted with EtOAc. The organic layer was dried over MgSO₄. Upon filtration and evaporation of the solvents under reduced pressure, a foam was isolated in quantitative yields.

NMR data was as follows:

¹H NMR (300 MHz, CDCl₃): δ =7.71 (bd, 2H), 7.34 (m, 7H), 7.20 (m, 1H), 7.01 (m, 2H), 6.80 (d, 2H, J=8.37 Hz), 5.27 (m, 1H), 4.75 (m, 1H), 4.04 (m, 1H), 3.23-2.93 (m, 4H), 2.42 (s, 3H), 1.85 (m, 1H), 1.60 (d, 3H, J=6.09 Hz), 1.36-1.26 (m, 3H).

¹³C NMR (75 MHz, CDCl₃): δ =174.74, 172.22, 157.53, 145.00, 143.77, 133.42, 130.76, 130.58, 129.14, 128.60, 128.48, 127.94, 126.15, 116.57, 76.39, 62.73, 53.90, 50.09, 37.09, 25.07, 24.52, 22.17.

Mass Spectroscopy: (FAB) 537 (M+H).

### EXAMPLE 370

### Synthesis of N-(Toluene-4-sulfonyl)-L-prolyl-4-(2-carboxyphenoxy)-L-phenylalanine

N-(Toluene-4-sulfonyl)-L-prolyl-L-tyrosine methyl ester (2.14 g, 5.16 mmol) was added to a suspension of sodium hydride, 60% in oil (1.1 eq., 228 mg) in xylenes (50 mL) at 0° C. The reaction mixture was stirred for 5 minutes and cuprous bromide dimethyl sulfide complex (1.4 eq., 1.48 g) was added. The reaction mixture was stirred at 23° C. for 0.5 hr. To this was added sodium 2-iodobenzoate (1.5 eq., 8.06 mmol), and the reaction mixture was refluxed for 12 hours. EtOAc (100 mL) was added, and the organic layer washed with NH₄Cl, 10% HCl, and brine, then dried over MgSO₄. The crude material was eluted on column chromatography (silica gel), with CHCl₃:MeOH (9:1), and isolated as an oil. The acid was prepared by treatment with NaOH (1.1 eq), in MeOH:H₂O (1:1) for 4 hours at room temperature. The diacid was isolated as a foam.

NMR data was as follows:

¹H NMR (300 MHz, CDCl₃): δ =7.71 (m, 2H), 7.29 (m, 4H), 7.19 (m, 4H), 6.72 (m, 1H), 4.84 (m, 1H), 4.13 (m, 1H), 3.39 (m, 1H), 3.11 (m, 3H), 2.43 (s, 3H), 1.89 (m, 1H), 1.48 (m, 3H).

¹³C NMR (75 MHz, CDCl₃): δ =172.67, 157.84, 155.89, 155.04, 145.17, 133.61, 133.19, 133.08, 131.69, 131.02, 130.64, 128.42, 127.87, 124.24, 120.04, 119.61, 116.12, 62.81, 50.31, 37.28, 30.69, 24.81, 22.15.

Mass Spectroscopy: (FAB) 553 (M+H).

### EXAMPLE 371

### Synthesis of N-(Toluene-4-sulfonyl)-L-prolyl-O-(benzyl)-L-tyrosine

N-(Toluene-4-sulfonyl)-L-Pro-OH was treated with (COCl)₂ and DMF in CH₂Cl₂ to give, after evaporation, N-(Toluene-4-sulfonyl)-L-Pro-Cl. This product was treated with L-Tyr(Bn)-OH and NaOH in THF and H₂O, to give, after acidification, extraction, drying with MgSO₄, and evaporation the title compound as a clear oil.

NMR data was as follows:

¹H NMR (DMSO-d₆, 300 MHz): δ=8.04 (d, J=8.2, 1H), 7.70 (d, J=8.1, 2H), 7.42-7.21 (m, 6H), 7.15 (d, J=8.5, 2H), 6.90 (d, J=8.5, 2H), 5.04 (s, 2H), 4.49-4.42 (m, 1H), 4.13-4.09 (m, 1H), 3.33-3.27 (m, 2H), 3.10-2.89 (m, 3H), 2.38 (s, 3H), 1.60-1.35 (m, 4H).

¹³C NMR (DMSO-d₆, 75 MHz): δ=172.63, 170.8, 157.0, 143.6, 137.2, 133.8, 130.3, 129.8, 129.4, 128.9, 128.4, 127.6, 125.3, 114.4, 69.1, 61.3, 53.4, 49.0, 35.8, 30.4, 23.8, 21.0.

Mass Spectroscopy: (+FAB, 3-nitrobenzyl alcohol) 523 (M+H).

### EXAMPLE 372

### Synthesis of N-(Toluene-4-sulfonyl)-L-prolyl-4-(1-H, 2-oxo-3-methyltetrahydropyrimidin-1-yl)-L-phenylalanine

The title compound was prepared from the corresponding t-butyl ester using the procedure described in Method 11.

NMR data was as follows:

¹H NMR (CDCl₃): δ =7.73 (d, 2H), 7.58 (d, 1H), 7.34 (d, 2H), 7.21 (d, 2H), 7.17 (d, 2H), 4.79 (q, 1H), 4.15-4.11 (m, 1H), 3.68-3.63 (m, 2H), 3.48-3.39 (m, 3H), 3.27 (dd, 1H), 3.17 (dd, 1H), 3.15-3.07 (m, 1H), 2.99 (s, 3H), 2.43 (s, 3H), 2.16-2.08 (m, 2H), 2.00-1.98 (m, 1H).

¹³C NMR (CDCl₃): δ=173.4, 172.2, 164.2, 156.4, 144.4, 142.5, 134.1, 133.0, 130.2, 130.0, 127.9, 126.2, 62.1, 53.4, 49.5, 48.9, 47.9, 36.5, 35.9, 30.2, 24.2, 22.0, 21.4.

### EXAMPLE 373

### Synthesis of N-(Toluene-4-sulfonyl)-L-prolyl-4-(2-methoxyphenyl)-L-phenylalanine

The title compound was prepared from the corresponding t-butyl ester using the procedure described in Method 11.

NMR data was as follows:

¹H NMR (CD₃OD): δ=7.70 (m, 2H), 7.36 (m, 4H), 7.22 (m, 4H), 6.98 (m, 2H), 4.75 (m, 1H), 4.10 (m, 1H), 3.71 (s, 3H), 3.29 (m, 2H), 3.11 (m, 2H), 2.39 (s, 3H), 1.75 (m, 1H), 1.53 (m, 3H).

¹³C NMR (CD₃OD): δ=174.4, 174.2, 158.1, 145.9, 138.9, 136.7, 135.1, 131.2, 130.9, 130.8, 130.2, 129.9, 129.1, 122.0, 112.6, 63.3, 55.9, 54.6, 50.5, 37.9, 31.5, 25.2, 21.4.

### EXAMPLE 374

### Synthesis of N-(Toluene-4-sulfonyl)-L-prolyl-4-(2-methoxyphenyl)-L-phenylalanine

The title compound was prepared from the corresponding t-butyl ester using the procedure described in Method 11.

NMR data was as follows:

¹H NMR (CDCl₃): δ 8.41 (d, 1H), 8.21 (s, 1H), 8.03 (d, 1H), 7.98 (s, 1H), 7.74 (d, 2H), 7.39 (d, 1H), 7.33 (d, 2H), 4.72-4.68 (m, 1H), 4.17-4.13 (m, 1H), 3.54-3.34 (m, 3H), 3.20-3.12 (m, 1H), 2.82 (s, 6H), 2.43 (s, 3H), 2.09-2.04 (m, 1H), 1.79-1.59 (m, 3H).

¹³C NMR (CDCl₃): δ=173.7, 171.8, 154.5, 147.2, 144.4, 137.8, 135.5, 133.2, 130.1, 127.9, 126.4, 62.2, 53.0, 49.5, 38.5, 36.0, 30.3, 24.4, 21.4.

### EXAMPLE 375

### Synthesis of N-(Toluene-4-sulfonyl)-L-prolyl-4-(2,4,5-trioxo-3-(3-chlorophenyl)-tetrahydroimidazol-1-yl)-L-phenylalanine benzyl ester

The compound was prepared by treatment of N-(toluene-4-sulfonyl)-L-prolyl-4-[(3-chlorophenyl ureido)-tetrahydroimidazol-1-yl]-L-phenylalanine isopropyl ester with oxalyl chloride in methylene chloride. The crude product was purified by flash chromatography (silica, 3:2 Hex: EtOAc) to afford a white solid. (0.410 g, 50%).

MS ((+) ESI, m/z (%) 746 (100[M+H]⁺) (746/748 1Cl)

### EXAMPLE 376

### Synthesis of N-(Phenyl-sulfonyl)-D-prolyl-L-4-(2,6-dimethoxyphenyl)phenylalanine

The title compound was prepared by coupling of 2,6-dimethoxyphenylboronic acid and 4′-iodophenylalanine derivates to provide dimethoxybiphenylalanines such as the title compound following procedures outlined in Hagmann et al., Bioorganic & Medicinal Chemistry Letters, 2001; 11(20): 2709-2713; Kamenecka et al., Bioorganic & Medicinal Chemistry Letters, 2002; 12(16): 2205-2208; and Doherty et al., Bioorganic & Medicinal Chemistry Letters, 2003; 13(11): 1891-1895.

### EXAMPLE 377

### Synthesis of N-(3,5-dichlorophenyl-sulfonyl)-D-prolyl-L-4-[4-(methylcarbonyl aminobutyl)-2,5-Dioxo-imidazolidin-1-yl]phenylalanine

The title compound was prepared following procedures outlined in WO 01/54690.

### EXAMPLE 378

### Synthesis of N-(2,6-dichlorophenyl-carbonyl)-L-4-(2,6-dimethoxyphenyl)phenylalanine

The title compound was prepared by coupling of 2,6-dimethoxyphenylboronic acid and 4′-iodophenylalanine derivates to provide dimethoxybiphenylalanines such as the title compound following procedures outlined in WO 99/36393 and Sircar et al., Bioorganic & Medicinal Chemistry, 2002; 10(6): 2051-2066.

### EXAMPLE 379

### Synthesis of N-[N-(3-pyridinesulfonyl)-L-3,3-dimethyl-4-thiaprolyl]-0-[1-methylpiperazin-4-ylcarbonyl]-L-tyrosine isopropyl ester

### 3-Pyridinesulfonyl chloride

The free base of the title compound may be prepared from 3-pyridinesulfonic acid (Aldrich) by a published procedure: Corey et al., J. Org. Chem. 1989, 54(2): 389. Alternatively, the hydrochloride of the title compound may be prepared from 3-pyridinesulfonic acid (Aldrich) by published procedures: Crowell et al., J. Med. Chem. 1989, 32(11): 2436; Karaman et al., J. Am. Chem. Soc. 1992, 114(12): 4889.

### L-3,3-Dimethyl-4-thiaproline

The title compound may be prepared from L-penicillamine (Aldrich) by published procedures: Samanen et al., J. Med. Chem. 1989, 32(2): 466; Nagasawa et al., J. Med. Chem. 1984, 27(5): 591.

### N-(3-pyridinesulfonyl)-L-3,3-dimethyl-4-thiaproline

A pH=7.4 buffer was prepared by dissolving disodium hydrogen phosphate (43.2 g, 0.304 mol) and potassium dihydrogen phosphate (11.8 g, 0.0870 mol) in H₂O to give a volume of 1.0 L. To a 0° C. solution of L-3,3-dimethyl-4-thiaproline (25.4 g, 0.157 mol) in 700 mL pH=7.4 buffer was added with stirring a solution of 3-pyridinesulfonyl chloride (28.0 g, 0.157 mol) in 300 mL CH₂Cl₂. The mixture was stirred for 24 h while warming to room temperature, and was acidified to pH=2 by addition of 3 M H₂SO₄, precipitating a yellow solid. The yellow solid was isolated by filtration of both phases, and the CH₂Cl₂ layer was separated and evaporated to afford additional yellow solid. The combined yellow solids were stirred in 700 mL H₂O for 1 h, to dissolve associated inorganic salts, and isolated again by filtration. The two aqueous layers were combined and extracted with EtOAc (3×500 mL). The EtOAc layers were washed with brine, treated with sodium sulfate, filtered, and evaporated to afford additional yellow solid. All aliquots of yellow solid were combined to afford 36.1 g (76%) N-(3-pyridinesulfonyl)-L-3,3-dimethyl-4-thiaproline.

### N-[N-(3-pyridinesulfonyl)-L-3,3-dimethyl-4-thiaprolyl]-L-tyrosine isopropyl ester

1-(3-Dimethylaminopropyl)-3-ethylcarbodiimide hydrochloride (4.83 g, 0.0253 mol) was added to a 0° C. solution of N-(3-pyridinesulfonyl)-L-3,3-dimethyl-4-thiaproline (6.37 g, 0.0211 mol), L-tyrosine isopropyl ester hydrochloride (5.48 g, 0.0211 mol), 1-hydroxybenzotriazole (5.69 g, 0.0421 mol), and 4-methylmorpholine (2.32 mL, 2.13 g, 0.0211 mol) dissolved in 125 mL DMF. The mixture was stirred for 16 h while warming to room temperature, and 200 mL EtOAc and 200 mL H₂O were added. The mixture was shaken, and the aqueous layer was separated, and the organic layer was washed with 0.2 M citric acid (2×100 mL), H₂O (2×100 mL), sat. NaHCO₃ (2×100 mL), H₂O (2×100 mL), and brine (2×100 mL). The organic layer was treated with sodium sulfate, filtered, and evaporated to afford 9.40 g (86%) N-[N-(3-pyridinesulfonyl)-L-3,3-dimethyl-4-thiaprolyl]-L-tyrosine isopropyl ester as a yellow foam. ¹H NMR (CDCl₃, 300 MHz) δ 9.08 (bs, 1H), 8.86 (bs, 1H), 8.16 (dt, J_{d}=8.1 Hz, Jₜ=2.0 Hz, 1H), 7.51 (dd, J=8.0 Hz, J=4.6), 7.07 (d, J=8.1 Hz, 2H), 6.87 (d, J=8.1 Hz, 1H), 6.74 (d, J=8.1 Hz, 2H), 5.96 (bs, 1H), 5.06 (sept, J=6.3, 1H), 4.83 (dt, J_{d}=6.0 Hz, Jₜ=7.8 Hz, 1H), 4.57 (d, J=9.3 Hz, 1H), 4.46 (d, J=9.3 Hz, 1H), 3.91 (s, 1H), 3.09 (dd, J=14.1 Hz, J=5.4 Hz, 1H), 2.98 (dd, J=14.1 Hz, J=7.5 Hz, 1H), 1.25 (t, J=6.6 Hz, 6H), 1.18 (s, 3H), 1.13 (s, 3H). ¹³C NMR (CDCl₃, 75 MHz) δ 170.5, 168.0, 155.2, 154.2, 148.6, 135.9, 130.7, 127.6, 124.1, 115.5, 105.5, 73.7, 69.7, 54.7, 53.4, 50.5, 37.5, 29.2, 23.7, 21.62, 21.55.

### N-[N-(3-pyridinesulfonyl)-L-3,3-dimethyl-4-thiaprolyl]-O-[1-methylpiperazin-4-ylcarbonyl]-L-tyrosine isopropyl ester

N-[N-(3-pyridinesulfonyl)-L-3,3-dimethyl-4-thiaprolyl]-L-tyrosine isopropyl ester (1.51 g, 2.89 mmol) and 4-nitrophenyl chloroformate (0.58 g, 2.89 mmol) were dissolved in 40 mL CH₂Cl₂, and the solution was stirred for 15 min while cooling in a −15° C. slurry of 4:1H₂O/EtOH and dry ice. To the solution was added Et₃N (1.00 mL, 0.73 g, 7.23 mol) with stirring over 2 min, and the solution was stirred for 1 h at −15° C. To the resulting suspension was added 1-methylpiperazine (0.32 mL, 0.289 g, 2.89 mmol) with stirring over 1 min, and the mixture was stirred for 16 h while warming to room temperature. The mixture was diluted with 40 mL hexanes, and washed with 10% (w/v) K₂CO₃ (4×50 mL) until no yellow color (4-nitrophenol) was seen in the aqueous layer. The organic layer was washed with brine (75 mL), treated with sodium sulfate, filtered, and evaporated to give a light yellow residue. The residue was purified by chromatography on silica gel using 70:25:5 CH₂Cl₂/EtOAc/EtOH to afford 1.53 g (84%) N-[N-(3-pyridinesulfonyl)-L-3,3-dimethyl-4-thiaprolyl]-O-[1-methylpiperazin-4-ylcarbonyl]-L-tyrosine isopropyl ester as a colorless foam. ¹H NMR (CDCl₃, 300 MHz) δ 9.09 (d, J=2.1 Hz, 1H), 8.87 (dd, J=4.9 Hz, J=1.6 Hz, 1H), 8.16 (dt, J_{d}=8.4 Hz, Jₜ=2.0 Hz, 1H), 7.51 (dd, J=8.2 Hz, J=4.9 Hz, 1H), 7.21 (d, J=8.4 Hz, 2H), 7.02 (d, J=8.4 Hz, 2H), 6.89 (d, J=7.8 Hz, 1H), 5.05 (sept, J=6.4 Hz, 1H), 4.84 (q, J=7.0 Hz, 1H), 6.59 (d, J=9.9 Hz, 1H), 4.47 (d, J=9.9 Hz, 1H), 3.90 (s, 1H), 3.67 (bs, 2H), 3.58 (bs, 2H), 3.18-3.03 (m, 2H), 2.45 (t, J=10.2 Hz, 4H), 2.34 (s, 3H), 1.26 (d, J=6.0 Hz, 3H), 1.23 (d, J=6.6 Hz, 3H), 1.20 (s, 3H), 1.17 (s, 3H). ¹³C NMR (CDCl₃, 75 MHz) δ 170.4, 167.8, 154.3, 153.7, 150.6, 148.7, 135.8, 133.1, 133.0, 130.4, 133.0, 121.8, 73.7, 69.7, 54.8, 54.6, 54.5, 50.5, 46.1, 44.3, 43.8, 37.6, 29.1, 23.8, 21.6, 21.5.

10.2 Synthesis of Compounds of Formulae III-IX

The following Methods may be used to prepare the compounds of formula

### Method A

### Methyl Ester Preparation Procedure

Amino acid methyl esters can be prepared using the method of Brenner and Huber Helv. Chim. Acta 1953, 36, 1109.

### Method B

### BOP Coupling Procedure

The desired dipeptide ester was prepared by the reaction of a carboxylic acid (1 equivalent) with the appropriate amino acid ester or amino acid ester hydrochloride (1 equivalent), benzotriazol-1-yloxy-tris(dimethylamino)phosphonium hexafluorophosphate [BOP] (2.0 equivalent), triethylamine (1.1 equivalent), and DMF. The reaction mixture was stirred at room temperature overnight. The crude product is purified flash chromatography to afford the dipeptide ester.

### Method C

### Hydrogenation Procedure I

Hydrogenation was performed using 10% palladium on carbon (10% by weight) in methanol at 30 psi overnight. The mixture was filtered through a pad of Celite and the filtrate concentrated to yield the desired compound.

### Method D

### Hydrolysis Procedure I

To a chilled (0° C.) THF/H₂O solution (2:1, 5-10 mL) of the appropriate ester was added LiOH (or NaOH) (0.95 equivalents). The temperature was maintained at 0° C. and the reaction was complete in 1-3 hours. The reaction mixture was extracted with ethyl acetate and the aqueous phase was lyophilized resulting in the desired carboxylate salt.

### Method E

### Ester Hydrolysis Procedure II

To a chilled (0° C.) THF/H₂O solution (2:1, 5-10 mL) of the appropriate ester was added LiOH (1.1 equivalents). The temperature was maintained at 0° C. and the reaction was complete in 1-3 hours. The reaction mixture was concentrated and the residue was taken up into H₂O and the pH adjusted to 2-3 with aqueous HCl. The product was extracted with ethyl acetate and the combined organic phase was washed with brine, dried over MgSO₄, filtered and concentrated to yield the desired acid.

### Method F

### Ester Hydrolysis Procedure III

The appropriate ester was dissolved in dioxane/H₂O (1:1) and 0.9 equivalents of 0.5 N NaOH was added. The reaction was stirred for 3-16 hours and then concentrated. The resulting residue was dissolved in H₂O and extracted with ethyl acetate. The aqueous phase was lyophilized to yield the desired carboxylate sodium salt.

### Method G

### BOC Removal Procedure

Anhydrous hydrochloride (HCl) gas was bubbled through a methanolic solution of the appropriate Boc-amino acid ester at 0° C. for 15 minutes and the reaction mixture was stirred for three hours. The solution was concentrated to a syrup and dissolved in Et₂O and reconcentrated. This procedure was repeated and the resulting solid was placed under high vacuum overnight.

### Method H

### tert-Butyl Ester Hydrolysis Procedure I

The tert-butyl ester was dissolved in CH₂Cl₂ and treated with TFA. The reaction was complete in 1-3 hr at which time the reaction mixture was concentrated and the residue dissolved in H₂O and lyophilized to yield the desired acid.

### Method I

### EDC Coupling Procedure I

To a CH₂Cl₂ solution (5-20 mL) of a carboxylic acid (1 equivalent), the appropriate amino acid ester hydrochloride (1 equivalent), N-methylmorpholine (1,1-2.2 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight. The reaction mixture was poured into H₂O and the organic phase was washed with sat. NaHCO₃, brine, dried (MgSO₄ or Na₂SO₄), filtered and concentrated. The crude product was purified by column chromatography.

### Method J

### EDC Coupling Procedure II

To a DMF solution (5-20 mL) of a carboxylic acid (1 equivalent), the appropriated amino acid ester hydrochloride (1 equivalent), Et₃N (1.1 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight. The reaction mixture was partitioned between EtOAc and H₂O and the organic phase washed with 0.2 N citric acid, H₂O, sat. NaHCO₃, brine, dried (MgSO₄ or Na₂SO₄), filtered and concentrated. The crude product was purified by column chromatography or preparative TLC.

### Method K

### tert-Butyl Ester Hydrolysis Procedure II

The tert-butyl ester was dissolved in CH₂Cl₂ (5 mL) and treated with TFA (5 mL). The reaction was complete in 1-3 hours at which time the reaction mixture was concentrated and the residue dissolved in H₂O and concentrated. The residue was redissolved in H₂O and lyophilized to yield the desired product.

### Method L

### Carbamate Formation Procedure I

Into a reaction vial were combined 15.2 mmol, 1.0 eq. of the starting hydroxy compound (typically a tyrosine derivative) and 1.86 g (15.2 mmol, 1.0 eq) DMAP. Methylene chloride (50 mL), triethylamine (2.12 mL, 1.54 g, 15.2 mmol, 1.0 eq), and dimethylcarbamyl chloride (1.68 mL, 1.96 g, 18.2 mmol, 1.2 eq) were then added. The vial was capped tightly, and the reaction solution swirled to obtain a homogeneous solution. The reaction solution was then heated to 40° C. After 48 h, TLC of the resulting colorless solution indicated complete conversion. The work-up of the reaction solution was as follows: 50 mL EtOAc and 50 mL hexanes was added to the reaction mixture, and the resulting mixture was washed with 0.5 M citric acid (3×50 mL), water (2×50 mL), 10% K₂CO₃ (2×50 mL), and sat. NaCl (1×50 mL); dried with MgSO₄, filtered and evaporated to afford the desired compound.

### Method M

### Carbamate Formation Procedure II

Into a reaction vial were combined 84.34 mmol (1.0 eq) of the starting hydroxy compound (typically a tyrosine derivative) and 17.0 g (84.34 mmol, 1.0 eq) 4-nitrophenyl chloroformate. Methylene chloride (700 mL) was added and the vial was capped with a septum. A nitrogen line was attached and the vial was immersed in a 4:1 water/ethanol dry ice slurry with stirring to cool to −15° C. Triethylamine (29.38 mL, 21.33 g, 210.81 mmol, 2.5 eq) was added over five minutes with stirring and the stirring was continued at −10 to −15° C. for 1 h. N-Methyl piperazine (9.35 mL, 8.45 g, 84.34 mmol, 1.0 eq) was added over three minutes with stirring and stirring was continued overnight while warming to room temperature. The reaction mixture was diluted with 700 mL hexanes and the resulting mixture was washed repeatedly with 10% K₂CO₃, until no yellow color (from 4-nitrophenol) is observed in the aqueous layer. The mixture was then washed with sat. NaCl, dried over anhydrous MgSO₄, filtered and evaporated. The residue was dissolved in 500 mL of ethanol and evaporated to remove triethylamine. The residue was again dissolved in 500 mL of ethanol and evaporated to remove triethylamine. The residue was then dissolved in 400 mL of ethanol and 600 mL of water was added with stirring to precipitate a solid or oil. If an oil if formed, the oil is stirred vigorously to induce it to solidify. The solid is then isolated by filtration. Dissolution, precipitation, and filtration are repeated once and the resulting solid is rinsed with water to remove traces of yellow color. The solid is then subjected to high vacuum until the mass remains constant thereby affording the desired carbamyloxy compound.

### Method N

### Preparation of 5-Iodo-4(3H)-pyrimidinone

The procedure of Sakamoto et. al. (Chem. Pharm. Bull. 1986, 34(7), 2719-2724) was used to convert 4(3H)-pyrimidinone into 5-iodo-4(3H)-pyrimidinone, which was of sufficient purity for conversion to 4-chloro-5-iodopyrimidine.

### Method O

### Preparation of 4-Chloro-5-iodopyrimidine

5-Iodo-4(3H)-pyrimidinone (1 eq.) was suspended in toluene to which was added POCl₃ (2.0 eq.). The reaction mixture was heated to reflux for 3 hours, and then cooled and concentrated. The residue was suspended in water, adjusted to pH=7 by addition of 4N sodium hydroxide, and extracted with ethyl acetate. The organic extracts were washed with brine, dried (MgSO₄), filtered and stripped to give a red oil. The crude product was dissolved in methanol and silica gel was added. Following concentration, the coated silica gel was loaded onto a plug of silica gel and elution with ethyl acetate/hexanes yielded the title compound.

### Method P

### Preparation of N-(5-Iodopydrimidin-4-yl)-L-4-(N,N-dimethylcarbamyloxy)phenylalanine tert-Butyl Ester

A solution 4-chloro-5-iodopyrimidine (1.0 eq.), L-4-(N,N-dimethylcarbamyloxy)-phenylalanine tert-butyl ester (1.0 eq), and N,N-diisoproylethyl amine (2.0 eq) in tetrahydrofuran was heated at reflux for 16 hours. The reaction mixture was then cooled and diluted with water and ethyl acetate. The organic phase was washed with 0.2 N citric acid, water, saturated NaHCO₃, brine, dried (MgSO₄), filtered and concentrated. The residue was purified by silica gel chromatography using ethyl acetate/hexanes to afford the title compound.

### Method Q

### Suzuki Coupling Procedure I

To an ethyleneglycol dimethyl ether solution of tetrakis(triphenylphosphine)palladium (0.04 eq) was added N-(5-iodopyrimidin-4-yl)-L-4-(N,N-dimethylcarbamyloxy)phenylalanine tert-butyl ester (1.0 eq.). After stirring for approximately ten minutes a boronic acid or ester (1.2 eq) and 2M Na₂CO₃ (2.0 eq) were added, and the reaction flask was evacuated and then flushed with nitrogen gas. The reaction was heated at reflux from three to sixteen hours. The reaction mixture was then cooled, diluted with water and ethyl acetate, and the organic phase was washed with 0.2 N citric acid, water, saturated NaHCO₃, brine, dried (MgSO₄), filtered and concentrated. Alternatively, the cooled reaction mixture was diluted with ethyl acetate and washed with water, saturated NaHCO₃, dried (MgSO₄), filtered and concentrated. Either column chromatography or preparative thin layer chromatography on silica gel using ethyl acetate/hexanes afforded the desired product.

### Method R

### Suzuki Coupling Procedure II

To a dimethylformamide solution of tetrakis(triphenylphosphine)-palladium (0.02-0.05 eq) was added N-(5-iodopyrimidin-4-yl)-L-4-(N,N dimethylcarbamyloxy)phenylalanine tert-butyl ester (1.0 eq.). After stirring for approximately ten minutes, the boronic acid (1,1-4.0 eq) and K₃PO₄ (1.5-2.0 eq) were added, and the reaction was heated at 100° C. for three to sixteen hours. The reaction mixture was then cooled, diluted with water and ethyl acetate, and the organic phase was washed with 0.2 N citric acid, water, saturated NaHCO₃, brine, dried (MgSO₄), filtered and concentrated. Either column chromatography or preparative thin layer chromatography on silica gel using ethyl acetate/hexanes afforded the desired product.

### Method S

### Suzuki Coupling Procedure III

An ethyleneglycol dimethyl ether/2M Na₂CO₃ (1:1 by volume) solution of tetrakis(triphenylphosphine)palladium (0.04 eq), N-(5-iodopyrimidin-4-yl)-L-4-(N,N-dimethylcarbamyloxy)phenylalanine tert-butyl ester (1.0 eq.), the boronic acid (1.1 eq) and lithium chloride (3.0 eq) was heated to reflux for approximately six hours. The cooled reaction mixture was diluted with ethyl acetate and washed with water, brine, dried (MgSO₄), filtered and concentrated. The residue was purified by silica gel column chromatography using ethyl acetate/hexanes to afford the desired product.

### Method T

### Suzuki Coupling Procedure IV

An ethyleneglycol dimethyl ether/2M Na₂CO₃, (1:1 by volume) solution of tetrakis(triphenylphosphine)palladium (0.05 eq), N-(5-iodopyrimidin-4-yl)-L-4-(N,N-dimethylcarbamyloxy)phenylalanine tert-butyl ester (1.0 eq.), the boronic acid (1.5 eq) and tri-o-tolylphosphine (0.1 eq) was heated to reflux for approximately three hours. The cooled reaction mixture was diluted with ethyl acetate and water and washed with water, brine, dried (MgSO₄), filtered and concentrated. The residue was purified by preparative thin layer chromatography on silica gel using ethyl acetate/hexanes to afford the desired product.

### Method U

### Heck Reaction Procedure I

A dimethylformamide solution of N-(5-iodopyrimidin-4-yl)-L-4-(N,N-dimethylcarbamyloxy)phenylalanine tert-butyl ester (1.0 eq.), N,N-dimethylacrylamide (2.0 eq), and triethylamine (6.0 eq) was degassed with nitrogen and then dichlorobis-(triphenylphosphine)palladium was added. The reaction was warmed to 90° C. under a stream of nitrogen for 16 hours. The cooled reaction mixture was diluted with ethyl acetate and water and washed with water, brine, dried (MgSO₄), filtered and concentrated. The residue was purified by column chromatography on silica gel using ethyl acetate/hexanes followed by preparative thin layer chromatography on silica gel using ethyl acetate/hexanes to afford the desired product.

### Method V

### Hydrogenation Procedure II

N-(5-(2-N,N-dimethylcarbamylethyl)pyrimidin-4-yl)-L-4-(N,N-dimethylcarbamyloxy)-phenylalanine tert-butyl ester was dissolved in ethanol to which was added 10% palladium on carbon. The reaction mixture was hydrogenated at 35 psi hydrogen for approximately five hours. The reaction mixture was filtered through a pad of Celite, and the filtrate was concentrated. The residue was purified by preparative thin layer chromatography on silica gel using methanol/dichloromethane to afford the desired product.

### Method W

### Heck Reaction Procedure II

To a tetrahydrofuran solution of N-(5-iodopyrimidin-4-yl)-L-4-(N,N-dimethylcarbamyloxy)phenylalanine tert-butyl ester (1.0 eq) dichlorobis(triphenylphosphine)palladium, triethylamine (0.05 eq) and triphenylphosphine (0.025 eq) was added phenylacetylene (1.5 eq) and triethylamine (1.5 eq). After twenty minutes, copper (1) iodide (0.012 eq) was added, and the resulting mixture was stirred overnight at room temperature. The reaction mixture was then diluted with ethyl acetate and water and washed with 0.2 N citric acid, water, saturated NaHCO₃, brine, dried (MgSO₄), filtered and concentrated. The residue was chromatographed on a silica gel column using ethyl acetate/hexanes. ¹H NMR analysis showed that the desired product to be contaminated with the iodopyrimidine starting material. However, the product was used without further purification.

### Method X

### Hydrogenation Procedure III

Crude N-(5-(2-phenylethynyl)pyrimidin-4-yl)-L-4-(N,N-dimethylcarbamyloxy)phenylalanine tert-butyl ester was dissolved in ethanol to which was added 10% palladium on carbon and sodium acetate (3.0 eq). The reaction mixture was hydrogenated at 40 psi hydrogen for approximately three hours, then filtered through a pad of Celite, and the filtrate concentrated. The residue was washed with 0.2 N citric acid, water, saturated NaHCO₃, brine, dried (MgSO₄), filtered and concentrated. Silica gel column chromatography using ethyl acetate/hexanes yielded the desired product.

### Method Y

### Preparation of N-(6-Chloropyrimidin-4-yl)-L-4-(N,N-dimethylcarbarnyloxy)phenylalanine tert-Butyl Ester

A solution 4,6-dichloropyrimidine (1.2 eq), L-4-(N,N-dimethylcarbamyloxy)-phenylalanine tert-butyl ester (1.0 eq), and triethylamine (1.05 eq) in ethanol was heated at reflux for 16 hours. The reaction mixture was cooled and concentrated, and the residue was taken-up in water and ethyl acetate. The organic phase was washed with 0.2 N citric acid, water, saturated NaHCO₃, brine, dried (MgSO₄), filtered and concentrated. The residue was purified by silica gel chromatography using ethyl acetate/hexanes to afford the title compound.

### Method Z

### Suzuki Coupling Procedure V

An ethyleneglycol dimethyl ether solution of tetrakis(triphenylphosphine)palladium (0.12 eq), N-(6-chloropyrimidin-4-yl)-L-4-(N,N-dimethylcarbamyloxy)phenylalanine tert-butyl ester (1.0 eq.) and triphenylphosphine (0.05 eq) was stirred for approximately ten minutes. The boronic acid or ester (1.2-2.5 eq) and 2M Na₂CO₃ (2.0 eq) were added, and the reaction was heated at 90° C. for 16 to 72 hours. The reaction mixture was cooled and concentrated, and the residue was taken up in water and ethyl acetate. The organic phase was washed with 0.2 N citric acid, water, saturated NaHCO₃, brine, dried (MgSO₄), filtered and concentrated. The residue was purified by preparative thin layer chromatography on silica gel using ethyl acetate/hexanes to afford the desired product.

### Method AA

### Preparation of N-(6-(N-Alkylamino)pyrimidin-4-yl)-L-4-(N,N-dimethylcarbamyloxy)phenylalanine tert-Butyl Ester

A mixture of N-(6-chloropyrimidin-4-yl)-L-4-(N,N-dimethylcarbamyloxy)phenylalanine tert-butyl ester (1.0 eq) and an alkylamine (10.0 eq) was heated in a sealed tube at 120° C. for 16 hours. The reaction mixture was cooled and diluted with ethyl acetate. The organic portion was washed with 0.2 N citric acid, water, saturated NaHCO₃, brine, dried (MgSO₄), filtered and concentrated. The residue was purified by silica gel chromatography using ethyl acetate/hexanes to afford the desired compound.

### Method BB

### Preparation of 4-N-Alkylamino-5-bromo-2-chloropyrimidine

A methanol solution of 5-bromo-2,4-dichloropyrimidine (1.0 eq), the alkylamine (1.05 eq, typically L-4-(N,N-dimethylcarbamyloxy)phenylalanine tert-butyl ester), and N,N-diisoproylethylamine (5.0 eq) was heated to 40° C. for 16 hours. The reaction mixture was then concentrated, and the residue was taken up in ethyl acetate. The organic portion was washed with 0.2 N citric acid, water, saturated NaHCO₃, brine, dried (MgSO₄), filtered and concentrated. The crude material was purified by silica gel chromatography using ethyl acetate/hexanes to afford the desired compound.

### Method CC

### Preparation of 4-N-Alkylamino-5-bromo-2-N-alkylaminopyrimidine

An isopropanol solution of the 4-N-alkylamino-5-bromo-2-chloropyrimidine (1.0 eq) and an alkylamine (5.0 eq) was heated in sealed tube at 130° C. for 3-5 hours. The reaction mixture was then cooled and washed with 0.2 N citric acid, water, saturated NaHCO₃, brine, dried (MgSO₄), filtered and concentrated. The crude material was purified by silica gel chromatography using ethyl acetate/hexanes to afford the desired compound.

### Method DD

### 4-N-Alkylamino-5-bromo-2-N-alkylaminopyrimidine Suzuki Coupling Procedure

To an ethyleneglycol dimethyl ether solution of tetrakis(triphenylphosphine)palladium (0.04 eq) was added an 4-N-alkylamino-5-bromo-2-N-alkylaminopyrimidine (1.0 eq.). After stirring for approximately ten minutes, the boronic acid or ester (1.2 eq) and 2M Na₂CO₃ (2.0 eq) was added, and the reaction flask was evacuated and then flushed with nitrogen gas. The reaction was heated at reflux for three to four hours. The reaction mixture was then cooled and diluted with water and ethyl acetate, and the organic phase was washed with 0.2 N citric acid, water, saturated NaHCO₃, brine, dried (MgSO₄), filtered and concentrated. The residue was purified by either silica gel column or preparative thin layer chromatography using ethyl acetate/hexanes to afford the desired product.

### Method EE

### Preparation of N-tert-Butoxycarbonyl-4-Iodo-L-phenyalanine Methyl Ester

The title compound was prepared from 4-iodo-L-phenylalanine by standard conditions described by Bodanszky and Bodanszky in The Practice of Peptide Synthesis; Springer-Verlag: Berlin, 1984.

### Method FF

### Preparation of N-tert-Butoxycarbonyl-4-(2,6-dimethoxyphenyl)-L-phenyalanine Methyl Ester

To a dimethylformamide solution of tetrakis(triphenylphosphine)palladium (0.02-0.05 eq) was added N-tert-butoxycarbonyl-4-(2,6-dimethoxyphenyl)-L-phenyalanine methyl ester (1.0 eq.). After stirring for approximately ten minutes, 2,6-dimethoxyphenyl boronic acid (1.1 eq) and K₃PO₄ (2.0 eq) were added, and the reaction was heated at 100° C. for sixteen hours. The reaction mixture was then cooled, diluted with water and ethyl acetate, and the organic phase was washed with 0.2 N citric acid, water, saturated NaHCO₃, brine, dried (MgSO₄), filtered and concentrated. Column chromatography on silica gel using ethyl acetate/hexanes afforded the desired product.

### Method GG

### Preparation of 4-(2,6-Dimethoxyphenyl)-L-phenyalanine Methyl Ester Trifluoroacetic Acid Salt

A methylene chloride solution of N-tert-butoxycarbonyl-4-(2,6-dimethoxyphenyl)-L-phenyalanine methyl ester was treated with trifluoroacetic acid for six hours at room temperature. Concentration of the volatiles yielded the title compound.

### Method HH

### tert-Butyl Ester Cleavage Procedure III

A methylene chloride solution of the appropriate tert-butyl ester was treated with trifluoroacetic acid at room temperature. After 2-3 hours the volatiles were evaporated, and the residue was treated again with methylene chloride and trifluoroacetic acid. After 2-3 hours the volatiles were evaporated again to yield the desired compound.

### Method II

### Preparation of N-(5-Allylpyrimidin-4-yl)-L-4-(N,N-dimethyl-carbamyloxy)phenylalanine tert-Butyl Ester

N-(5-Iodo-pyrimidin-4-yl)-L-4-(N,N-dimethyl-carbamyloxy)phenylalanine tert-butyl ester (1.0 eq) was dissolved in dry DMF, with allyltributylstannane (1.1 eq), bis(triphenylphosphine)palladium dichloride (0.03 eq) and LiCl (3.0 eq). The reaction mixture was flushed under nitrogen, and heated to 90° C. for 2 hours. EtOAc was added, and the organic layer was washed with water and brine, and dried over MgSO₄. After filtration and evaporation of the solvent under reduced pressure, the crude material was purified by column chromatography (silica gel) eluting with EtOAc/hexanes 1:3. The title material was isolated in good yields.

### Method JJ

### Preparation of N-[5-propylpyrimidin-4-yl]-L-4-(N,N-dimethyl-carbamyloxy)phenylalanine tert-Butyl Ester

N-(5-Allylpyrimidin-4-yl)-L-4-(N,N-dimethylcarbamyloxy)phenylalanine tert-butyl ester was dissolved in methanol and treated with a catalytic amount of 10% palladium on carbon. The mixture was shaken under 10 psi hydrogen gas for 3 hours. Upon filtration though a pad of Celite, and evaporation of the solvent under reduced pressure, the desired material was isolated as a foam.

### Method KK

### Preparation of N-(5-propylpyrimidin-4-yl)-L-4-(N,N-dimethyl-carbamyloxy)phenylalanine

N-(5-Propylpyrimidin-4-yl)-L-4-(N,N-dimethylcarbamyloxy)-phenylalanine tert-butyl ester was treated with neat trifluoroacetic acid, and the mixture was stirred for 5 h at room temperature. Upon evaporation of the solvent under reduced pressure, the desired material was isolated as a foam.

### Method LL

### Preparation of Dimethyl 2-Alkylmalonate

To a suspension of sodium hydride 60% dispersion in mineral oil (1.1 eq) in anhydrous THF was added slowly with stirring dimethyl malonate (1.1 eq), causing the evolution of gas. To the resulting solution was added a bromoalkane, iodoalkane, or trifluoromethanesulfonyloxyalkane (1.0 eq), and the mixture was heated to 50° C. for 48 h, at which point TLC indicated consumption of the bromoalkane, iodoalkane, or trifluoromethanesulfonyloxyalkane. The mixture was diluted with diethyl ether and washed with 70% saturated sodium chloride. The organic extracts were treated with anhydrous magnesium sulfate, filtered, and evaporated to afford a dimethyl 2-alkylmalonate of sufficient purity for immediate conversion to a 5-alkyl-4,6-dihydroxypyrimidine.

### Method MM

### Preparation of Diethyl 2-Alkylidenylmalonate

Procedure B (p. 2759) of Houve and Winberg (J. Org. Chem. 1980, 45(14), 2754-2763) was employed to react diethyl malonate with a ketone or an aldehyde to afford a diethyl 2-alkylidenylmalonate of sufficient purity for immediate conversion to a diethyl 2-alkylmalonate.

### Method NN

### Preparation of Diethyl 2-Alkylmalonate

A diethyl 2-alkylidenylmalonate and an equal mass 10% palladium on carbon were suspended in ethanol. The mixture was shaken under 55 psi hydrogen gas for 24 h, at which point TLC indicated consumption of the diethyl 2-alkylidenylmalonate. The mixture was filtered through Celite and evaporated to afford a diethyl 2-alkylmalonate of sufficient purity for immediate conversion to a 5-alkyl-4,6-dihydroxypyrimidine.

### Method OO

### Preparation of 5-Alkyl-4,6-dihydroxypyrimidine

To a diethyl 2-alkylmalonate or a dimethyl 2-alkylmalonate (1.0 eq) was added formamidine acetate (1.0 eq) and 25% sodium methoxide in methanol (3.3 eq). The resulting slurry was stirred vigorously and heated to 60° C. for 4 h, and then allowed to cool. The slurry was diluted with water, and acidified to pH=2 by addition of HCl. The resulting precipitate was collected by filtration, washed with water, and dried under vacuum, to afford a 5-alkyl-4,6-dihydroxypyrimidine of sufficient purity for immediate conversion to a 5-alkyl-4,6-dichloropyrimidine.

### Method PP

### Preparation of 5-Alkoxy-4-hydroxypyrimidine

The method (p. 308) of Anderson et al. (Org. Proc. Res. Devel. 1997, 1, 300-310) was employed to react a methyl alkoxyacetate, sodium methoxide, ethyl formate, and formamidine acetate to afford a 5-alkoxy-4-hydroxypyrimidine of sufficient purity for immediate conversion to a 5-alkoxy-4-chloropyrimidine.

### Method QQ

### Preparation of 5-Alkyl-4,6-dichloropyrimidine or 5-Alkoxy-4-chloropyrimidine

To a 5-alkyl-4,6-dihydroxypyrimidine or a 5-alkoxy-4-hydroxypyrimidine (1.0 eq) were added phosphorus oxychloride (15.0 eq) and N,N-dimethylaniline (1.0 eq), and the mixture was heated to 100° C. for 3 h, and then allowed to cool. The resulting solution was poured onto ice, and the mixture was extracted with dichloromethane. The organic extracts were treated with anhydrous magnesium sulfate, filtered, and evaporated to afford a 5-alkyl-4,6-dichloropyrimidine or a 5-alkoxy-4-chloropyrimidine of sufficient purity for immediate conversion to a 5-alkyl-4-N-alkylamino-6-chloropyrimidine or a 5-alkoxy-4-N-alkylaminopyrimidine.

### Method RR

### Preparation of 5-Alkyl-4-N-alkylamino-6-chloropyrimidine or 5-Alkoxy-4-N-alkylaminopyrimidine

To a solution of a 5-alkyl-4,6-dichloropyrimidine or a 5-alkoxy-4-chloropyrimidine (1.0 eq) in ethanol were added an alkyl amine (1.2 eq, typically L-4-(N,N-dimethylcarbamyloxy)-phenylalanine tert-butyl ester) and diisopropylethylamine (2.0 eq). The mixture was sealed in a pressure tube and heated to 120° C. for 48 h, at which point TLC indicated consumption of the 5-alkyl-4,6-dichloropyrimidine or the 5-alkoxy-4-chloropyrimidine. The mixture was evaporated, and the residue was partitioned between ethyl acetate and pH=4.5 citrate buffer. The organic extracts were washed with saturated sodium chloride, treated with anhydrous magnesium sulfate, filtered, and evaporated. The residue was purified by chromatography on silica gel using ethyl acetate and hexanes to afford a pure 5-alkyl-4-N-alkylamino-6-chloropyrimidine or 5-alkoxy-4-N-alkylaminopyrimidine.

### Method SS

### Preparation of 5-Alkyl-4-N-alkylaminopyrimidine (Procedure I)

A suspension of 5-alkyl-4-N-alkylamino-6-chloropyrimidine (1.0 eq), and an equal mass 10% palladium on carbon, and sodium bicarbonate (5.0 eq) in methanol was shaken under 55 psi hydrogen gas for 16 h, at which point TLC indicated consumption of the 5-alkyl-4-N-alkylamino-6-chloropyrimidine. The mixture was filtered through Celite and evaporated to give a residue, which was partitioned between ethyl acetate and 70% saturated sodium chloride. The organic extracts were treated with anhydrous magnesium sulfate, filtered, and evaporated. The residue was purified by chromatography on silica gel using ethyl acetate and hexanes to afford a pure 5-alkyl-4-N-alkylaminopyrimidine.

### Method TT

### Preparation of 5-Alkyl-4-N-alkylaminopyrimidine (Procedure II)

A suspension of 5-alkyl-4-N-alkylamino-6-chloropyrimidine (1.0 eq), sodium acetate (10.0 eq), and zinc powder (20.0 eq) in a 9:1 mixture of acetic acid and water was stirred vigorously at 40° C. for 72 h, at which point TLC indicated partial consumption of the 5-alkyl-4-N-alkylamino-6-chloropyrimidine. The supernatant solution was decanted from remaining zinc and evaporated. The residue was partitioned between ethyl acetate and saturated sodium bicarbonate, and the organic extracts were treated with anhydrous magnesium sulfate, filtered, and evaporated. The residue was purified by chromatography on silica gel using ethyl acetate and hexanes to afford a pure 5-alkyl-4-N-alkylaminopyrimidine.

### Method UU

### Preparation of N-Benzyloxycarbonyl-L-Tyrosine tert-Butyl Ester

To a 0° C. suspension of L-tyrosine tert-butyl ester (Bachem, 1.0 eq) and sodium bicarbonate (2.0 eq) in a 1:1 mixture of THF and water was added slowly with stirring benzyl chloroformate (1.1 eq). After the addition, the mixture was stirred at 0° C. for 3 h and at room temperature for 24 h. The mixture was diluted with diethyl ether, and the aqueous layer was separated. The organic extracts were washed with saturated sodium chloride, treated with anhydrous magnesium sulfate, filtered, and evaporated to afford N-benzyloxycarbonyl-L-tyrosine tert-butyl ester of sufficient purity for immediate conversion of the tyrosine hydroxyl into a carbamate.

### Method VV

### Preparation of N-Benzyloxycarbonyl-L-4-(N,N-Dimethylcarbamyloxy)phenylalanine tert-Butyl Ester

A mixture of N-benzyloxycarbonyl-L-tyrosine tert-butyl ester (1.0 eq), 4-dimethylaminopyridine (1.0 eq), triethylamine (1.5 eq), dimethylcarbamylchloride (1.2 eq), and dichloromethane was heated to 37° C. for 16 h. The mixture was diluted with additional dichloromethane and washed sequentially with 1.0 M potassium bisulfate, water, saturated sodium bicarbonate, and saturated sodium chloride. The organic extracts were treated with anhydrous magnesium sulfate, filtered, and evaporated to afford N-benzyloxycarbonyl-L-4-(N,N-dimethylcarbamyloxy)phenylalanine tert-butyl ester as a white solid of sufficient purity for immediate conversion to L-4-(N,N-dimethylcarbamyloxy)phenylalanine tert-butyl ester.

### Method WW

### Preparation of L-4-(N,N-Dimethylcarbamyloxy)phenylalanine tert-Butyl Ester

A suspension of N-benzyloxycarbonyl-L-4-(N,N-dimethylcarbamyloxy)phenylalanine tert-butyl ester and an equal mass of 10% palladium on carbon in methanol was shaken under 55 psi hydrogen gas for 1 h, at which point TLC indicated consumption of the N-benzyloxycarbonyl-L-4-(N,N-dimethylcarbamyloxy)phenylalanine tert-butyl ester. The mixture was filtered through Celite and evaporated to afford L-4-(N,N-dimethylcarbamyloxy)-phenylalanine tert-butyl ester of sufficient purity for immediate use in reactions with chloropyrimidines.

### Method XX

### Preparation of N-Benzloxycarbonyl-L-4-(4-methylpiperazin-1-ylcarbonyloxy)phenylalanine tert-Butyl Ester

To a stirred solution maintained at 0° C. of N-benzyloxycarbonyl-L-tyrosine tert-butyl ester (1.0 eq) and triethylamine (2.5 eq) in dichloromethane was added 4-nitrophenyl chloroformate (1.0 eq). The mixture was stirred for 30 min at 0° C., and then 1-methylpiperazine (1.5 eq) was added, and then the mixture was stirred for 2 h while warming to room temperature. The mixture was diluted with ethyl acetate and washed five times with 10% potassium carbonate and once with saturated sodium chloride. The organic extracts were treated with anhydrous magnesium sulfate, filtered, and evaporated to afford N-benzyloxycarbonyl-L-4-(4-methylpiperazin-1-ylcarbonyloxy)phenylalanine tert-butyl ester of sufficient purity for immediate conversion to L-4-(4-methylpiperazin-1-ylcarbonyloxy)phenylalanine tert-butyl ester.

### Method YY

### Preparation of L-4-(4-Methylpiperazin-1-ylcarbonyloxy)phenylalanine tert-Butyl Ester

A suspension of N-benzyloxycarbonyl-L-4-(4-methylpiperazin 1-ylcarbonyloxy)-phenylalanine tert-butyl ester and an equal mass of 10% palladium on carbon in methanol was shaken under 55 psi hydrogen gas for 1 h, at which point TLC indicated consumption of N-benzyloxycarbonyl-L-4-(4-methylpiperazin-1-ylcarbonyloxy)phenylalanine tert-butyl ester. The mixture was filtered through Celite and evaporated to afford L-4-(4-methylpiperazin-1-ylcarbonyloxy)phenylalanine tert-butyl ester of sufficient purity for immediate use in reactions with chloropyrimidines.

### Method ZZ

### tert-Butyl Ester Cleavage Procedure IV

The tert-butyl ester was dissolved in 96% formic acid and heated to 40° C. for 16 h, at which point TLC indicated consumption of the tert-butyl ester. The mixture was evaporated under a stream of air to give a residue, which was stored under high vacuum for 72 h to afford the pure carboxylic acid.

### Method AAA

### Preparation of 2,4-Dichloro-5-nitropyrimidine

5-Nitrouracil was treated with phosphorus oxychloride and N,N-dimethylaniline, according to the procedure of Whittaker (J. Chem. Soc. 1951, 1565), to give 2,4-dichloro-5-nitropyrimidine as an orange oil, which was used without distillation immediately in the next step.

### Method BBB

### Preparation of N-(2-Chloro-5-nitropyrimidin-4-yl)-L-4-(N,N-dimethylcarbamyloxy)phenylalanine tert-Butyl Ester

To a stirred solution of L-4-(N,N-dimethylcarbamyloxy)phenylalanine tert-butyl ester (6.38 g, 20.69 mmol) and N,N-diisopropylethylamine (5.40 mL, 4.01 g, 31.03 mmol) in 70 mL CH₂Cl₂ at 0° C., was added a solution of 2,4-dichloro-5-nitropyrimidine (3.25 g, 20.69 mmol) in 70 mL of CH₂Cl₂, at such a rate the temperature did not exceed 10° C. After the addition, the mixture was stirred at 0-10° C. for 15 minutes, at which point TLC indicated conversion of 2,4-dichloro-5-nitropyrimidine. To the mixture were added 100 mL 1 \ddot{M} KHSO₄ and 200 mL diethyl ether. The organic layer was separated, washed (H₂O, sat. NaHCO₃, and sat. NaCl), dried (MgSO₄), filtered, and evaporated to give N-(2-chloro-5-nitropyrimidin-4-yl)-L-4-(N,N-dimethylcarbamyloxy)phenylalanine tert-butyl ester (9.52 g, 20.45 mmol, 99%) as an orange oil, which was used immediately in the next step.

### Method CCC

### Preparation of N-(5-Aminopyrimidin-4-yl)-L-4-(N,N-dimethylcarbamyloxy)phenylalanine tert-Butyl Ester

A mixture of N-(2-chloro-5-nitropyrimidin-4-yl)-L-4-(N,N-dimethylcarbamyloxy)-phenylalanine tert-butyl ester (9.52 g, 20.45 mmol), Degussa-type 20% palladium on carbon (9.52 g), NaHCO₃ (8.59 g, 102.2 mmol), and 165 mL MeOH was shaken under 55 psi H₂ for 16 h, at which point TLC indicated conversion of N-(2-chloro-5-nitropyrimidin-4-yl)-L-4-(N,N-dimethylcarbamyloxy)phenylalanine tert-butyl ester into a single product. The mixture was filtered through Celite, and the filtrate was evaporated to give a residue, which was dissolved by addition of 150 mL EtOAc and 75 mL H₂O. The organic layer was separated, washed (sat. NaCl), dried (MgSO₄), filtered, and evaporated to give N-(5-aminopyrimidin-4-yl)-L-4-(N,N-dimethylcarbamyloxy)phenylalanine tert-butyl ester (7.14 g, 17.79 mmol, 87%) as an orange solid, which was used immediately in the next step.

### Method DDD

### Preparation of N-(5-(N-4-Toluenesulfonylamino)pyrimidin-4-yl)-L-4-(N,N-dimethylcarbamyloxy)phenylalanine tert-Butyl Ester

To a stirred solution of N-(5-aminopyrimidin-4-yl)-L-4-(N,N-dimethylcarbamyloxy)-phenylalanine tert-butyl ester (1.00 g, 2.49 mmol) in 10 mL anhydrous pyridine at 0° C., was added in portions 4-toluenesulfonylchloride (0.474 g, 2.49 mmol). After the addition, the resulting red solution was stirred at 0° C. for 3 h, at which point TLC indicated nearly complete conversion of N-(5-aminopyrimidin-4-yl)-L-4-(N,N-dimethylcarbamyloxy)phenylalanine tert-butyl ester. To the mixture was added 3-dimethylaminopropylamine (0.325 mL, 0.264 g, 2.49 mmol), and the mixture was stirred for 30 min while warming to room temperature. The mixture was poured into 100 mL 1 M KHSO₄, and extracted with 150 mL EtOAc. The organic layer was washed (2×1 \ddot{M} KHSO₄, H₂O, sat. NaHCO₃, sat. NaCl), dried (MgSO₄), filtered, and evaporated to give a brown residue, which was purified by flash chromatography using EtOAc/hexanes on silica gel, to give N-(5-(N-4-toluenesulfonylamino)pyrimidin-4-yl)-L-4-(N,N-dimethylcarbamyloxy)phenylalanine tert-butyl ester (1.01 g, 1.81 mmol, 73%) as a clear oil.

### Method EEE

### Preparation of N-(5-(N-Methyl-N-4-toluenesulfonylamino)pyrimidin-4-yl)-L-4-(N,N-dimethylcarbamyloxy)phenylalanine tert-Butyl Ester

To a stirred two-phase mixture of 45 mL 1 \ddot{M} NaOH and 25 mL diethyl ether at 0° C., was added in portions 1-methyl-3-nitro-1-nitrosoguanidine (1.33 g, 9.05 mmol). After stirring for 25 min, at which point evolution of N₂ had subsided, the bright yellow solution of diazomethane in diethyl ether was transferred by pipette to a stirred solution of N-(5-(N-4-toluenesulfonylamino)pyrimidin-4-yl)-L-4-(N,N-dimethylcarbamyloxy)phenylalanine tert-butyl ester (1.01 g, 1.81 mmol) in 15 mL diethyl ether and 15 mL CH₂Cl₂ at 0° C. After stirring for 15 min, at which point TLC indicated complete conversion of N-(5-(N-4-toluenesulfonylamino)pyrimidin-4-yl)-L-4-(N,N-dimethylcarbamyloxy)phenylalanine tert-butyl ester, excess AcOH was added to destroy unreacted diazomethane. The mixture was diluted with 100 mL diethyl ether, washed (2×sat. NaHCO₃, sat. NaCl), dried (MgSO₄), filtered and evaporated to give a yellow residue, which was purified by flash chromatography using EtOAc/hexanes on silica gel, to give N-(5-(N-methyl-N-4-toluenesulfonylamino)pyrimidin-4-yl)-L-4-(N,N-dimethylcarbamyloxy)phenylalanine tert-butyl ester (0.846 g, 1.48 mmol, 82%) as a clear oil.

### Method FFF

### Preparation of Diethyl 2-(N,N-Dialkylamino)malonate

The appropriate amine (1.0 eq) was added to a 0° C. solution of diethyl bromomalonate (1.0 eq) and N,N-diisopropylethyl amine (1.1 eq) in ethanol. The mixture was stirred and allowed to warm room temperature. After 16 hours, the reaction mixture was concentrated and the residue was suspended in ethyl acetate and sat. NaHCO₃. The organic portion was washed with sat NaHCO₃, brine, dried (MgSO₄) filtered and concentrated to yield the diethyl 2-(N,N-dialkylamino)malonate, of sufficient purity for immediate conversion to a 5-(N,N-dialkylamino)-4,6-dihydroxypyrimidine.

### Method GGG

### Preparation of 5-(N,N-Dialkylamino)-4,6-dihydroxypyrimidine

A suspension of a diethyl 2-(N,N-dialkylamino)malonate (1.0 eq), formamidine acetate (1.10 eq.) and 25% sodium methoxide in methanol (3.3 eq) was heated to 65° C. for 3.5 hours. The reaction mixture was cooled and diluted with water. The mixture was acidified to pH=4.5 by addition of dilute HCl. The resulting precipitate was collected by filtration, washed with water, and dried under vacuum to afford a 5-(N,N-dialkylamino)-4,6-dihydroxypyrimidine of sufficient purity for immediate conversion to a 5-(N,N-dialkylamino)-4,6-dichloropyrimidine. Alternatively, the acidified solution was evaporated to give a solid residue, which was extracted with boiling ethanol. The ethanol extracts were filtered and concentrated to give a residue, which was recrystallized from isopropyl alcohol to afford a 5-(N,N-dialkylamino)-4,6-dihydroxypyrimidine of sufficient purity for immediate conversion to a 5-(N,N-dialkylamino)-4,6-dichloropyrimidine.

### Method HHH

### Preparation of 5-(N,N-Dialkylamino)-4,6-dichloropyrimidine

A 5-(N,N-dialkylamino)-4,6-dihydroxypyrimidine (1.0 eq) was suspended in POCl₃ (15.0 eq), and the mixture was heated to reflux for 16 hours. Then the mixture was cooled and carefully poured into a suspension of ethyl ether and aqueous K₂CO₃. The organic portion was washed with brine, dried (MgSO₄), filtered and concentrated to yield a 5-(N,N-dialkylamino)-4,6-dichloro-pyrimidine of sufficient purity for immediate reaction with alkylamines.

### Method III

### Preparation of 4-(N-Alkylamino)-5-(N,N-dialkylamino)-6-chloropyrimidine

A 5-(N,N-dialkylamino)-4,6-dichloropyrimidine (1.0 eq), L-4-(N,N-dimethylcarbamyloxy)phenylalanine tert-butyl ester (1.5 eq) and N,N-diisopropyl ethylamine (1.5 eq) were dissolved in ethanol and heated to 120° C. in a sealed tube for 72 h. The cooled reaction mixture was concentrated, and the residue dissolved in ethyl acetate. The ethyl acetate solution was washed with 0.2 N citric acid, water, saturated NaHCO₃, brine, dried (MgSO₄), filtered and concentrated. The residue was purified by silica gel chromatography using ethyl acetate/hexanes to afford the 4-(N-alkylamino)-5-(N,N-dialkylamino)-6-chloropyrimidine.

### Method JJJ

### Preparation of 4-(N-Alkylamino)-5-(N,N-dialkylamino)pyrimidine

A 4-(N-Alkylamino)-5-(N,N-dialkylamino)-6-chloropyrimidine (1.0 eq), an equal mass of 10% palladium on carbon. and NaHCO₃ (5.0 eq) were suspended in methanol. The reaction mixture was hydrogenated at 45 psi hydrogen for 16 hours and then filtered through a pad of Celite. The filtrate was concentrated, and the residue was dissolved in ethyl acetate. The ethyl acetate solution was washed with water, brine, dried (MgSO₄), filtered and concentrated to yield an oil. The oil was purified by column chromatorgraphy on silica gel using ethyl actate and hexanes to afford a pure 4-(N-alkylamino)-5-(N,N-dialkylamino)pyrimidine.

### Method KKK

### Suzuki Coupling Procedure V

To an ethyleneglycol dimethyl ether solution of tetrakis(triphenylphosphine) palladium (0.04 eq) was added N-(5-bromo-2-chloro-pyrimidin-4-yl)-L-4-(N,N-dimethylcarbamyloxy)phenylalanine tert-butyl ester (1.5 eq.). After stirring for approximately ten minutes o-tolylboronic acid (1.5 eq) and 2M Na₂CO₃ (2.0 eq) were added, and the reaction flask was evacuated and flushed with nitrogen gas. The reaction was heated tp reflux for four hours. The reaction mixture was then cooled and diluted with water and methylene chloride. The organic phase was separataed and washed with brine, dried (MgSO₄), filtered and concentrated. The residue was purified by silica gel chromatography using ethyl acetate/hexanes to afford the desired product.

### Method LLL

### Preparation of L-Phenylalanine Isopropyl Ester Hydrochloride or

L-Tyrosine Isopropyl Ester Hydrochloride

Excess HCl gas was added with stirring to a suspension of L-phenylalanine or L-tyrosine in excess isopropanol. The mixture was heated to reflux for 16 h, and then the volatiles were evaporated under vacuum to give L-phenylalanine isopropyl ester hydrochloride or L-tyrosine isopropyl ester hydrochloride of sufficient purity for immediate use.

### Method MMM

### Bromopyrimidine Debromination Procedure

The bromopyrimidine was dissolved in isopropyl alcohol to which was added 10% palladium on carbon. The reaction was hydrogenated at 45 psi hydrogen. Filtration and concentration of the filtrate yielded the desired dehalogenated pyrimidine.

### Method NNN

### Preparation of 2-Isopropropoxypyrimidine

A 2-chloropyrimidine was dissolved in isopropyl alcohol to which was added diisopropylamine. The reaction was heated in a sealed tube for ten days at 130° C. The cooled reaction mixture was concentrated, and the product purified via silica gel column chromatography to yield the 2-isopropoxypyrimidine.

### Method OOO

### Heck Reaction Procedure III

To a dioxane/triethylamine (1:1 by volume) solution of the N-(5-iodopyridin-4-yl)-L-4-(N,N-dimethylcarbamyloxy)phenylalanine isopropyl ester (1.0 eq), triphenylphosphine (0.05 eq), copper (I) iodide (0.2 eq) was added phenylacetylene (4.0 eq). After flushing the solution for ten minutes with nitrogen gas, dichlorobis(triphenylphosphine)palladium (0.10 eq) was added, and the resulting reaction mixture heated to 50° C. for 16 hours. The reaction mixture was then diluted with ethyl acetate and water, and the organic portion was washed with 0.2 N citric acid, water, saturated NaHCO₃, brine, dried (MgSO₄), filtered and concentrated. The residue was chromatographed on a silica gel column using ethyl acetate/hexanes to afford the desired product.

### Method PPP

### Preparation of N-[5-(Phenyl)pyrimidin-4-yl]-L-4-(N,N-dimethylcarbamyloxy)phenylalanine tert-Butyl Ester

N-[5-iodopyrimidin-4-yl]-L-4-(N,N-dimethylcarbamyloxy)phenylalanine tert-butyl ester (123 mg, 0.2 mmol) was diluted in dry DMF (5 mL) under nitrogen with KOAc (3.0 eq, 73 mg), bis(pinacolato)diboron (1.1 eq, 63 mg), and a catalytic amount of [1,1′-bis(diphenylphosphino)-ferrocene]dichloropalladium (II) complex with dichloromethane (1:1). The reaction was heated for 2 hours at 100° C. To this was added, K3PO4 (2.0 eq, 105 mg), iodobenzene (2.0 eq, 0.056 mL) and an additional catalytic amount of [1,1′-bis(diphenylphosphino)-ferrocene]dichloropalladium (II) complex with dichloromethane (1:1). The reaction mixture was stirred overnight at 100° C. EtOAc was added and the organic layer washed with brine, dried over MgSO₄. Upon filtration, and evaporation of the solvent under reduced pressure, the crude material was eluted on column chromatography (silica gel) with EtOAc/hexanes 1:1. The desired material was isolated in good yields.

### Method QQQ

### Preparation of 2-Amino-3-Chloropyrazine

A mixture of 2,3-dichloropyrazine (Lancaster) and ammonium hydroxide was heated in a sealed tube at 100° C. for 24 h resulting in a white precipitate. The precipitate was collected by filtration and dried under vacuum to afford 2-amino-3-chloropyrazine of sufficient purity for immediate conversion to 2-chloro-3-nitropyrazine.

### Method RRR

### Preparation of 2-Chloro-3-Nitropyrazine

The method (p. 1638) of Hartman et al. (J. Med. Chem. 1984, 27(12), 1634-1639) was employed to convert 2-amino-3-chloropyrazine into 2-chloro-3-nitropyrazine of sufficient purity for immediate use.

### Method SSS

### Preparation of 4-Alkylamino-2-dialkylamino-5-nitropyrimidine

A solution of 1.0 eq 4-alkylamino-2-chloro-5-nitropyrimidine and 5.0 eq dialkylamine in THF was allowed to stand for 16 h. The mixture was diluted with ethyl acetate and then washed with pH=4.5 citrate buffer and saturated sodium chloride. The organic extracts were treated with anhydrous magnesium sulfate, filtered, and evaporated to give a residue, which was purified by chromatography on silica gel using ethyl acetate and hexanes.

### Method TTT

### Preparation of L-4-(2,6-Dimethoxyphenyl)phenylalanine Methyl Ester

To a stirred solution (DMF, 66 mL) of N-Boc-L-φ-iodo)phenylalanine methyl ester (13.2 g, 32.7 mmol) prepared according to the procedure of Schwabacher et al., J. Org. Chem. 1994, 59, 4206-4210) was added Pd(PPh₃)₄ (0.03 eq, 1.13 g, 1 mmol). The solution was stirred for 10 min and then 2,6-dimethoxyboronic acid (1.2 eq, 7.1 g, 39 mmol) and K₃PO₄ (1.5 eq, 10.4 g, 49 mmol) were added. The reaction flask was evacuated and flushed with nitrogen. This process was repeated twice and the reaction mixture was then heated to 100° C. under a stream of nitrogen for about 3.5 h at which time TLC showed the reaction to be complete (4.5:1 hexanes:EtOAc, R^{f}=0.2, IV active). The reaction mixture was cooled and partitioned between water and ethyl acetate (200 mL each). The organic portion was washed with 0.2N citric acid (3×100 mL), brine (1×100 mL), dried (MgSO₄), filtered and stripped to a thick reddish oil, about 13 g. The resulting product was chromatographed on silica gel eluting with 4.5:1 hexanes/EtOAc, R^{f}=0.2. The combined fractions were stripped and treated with methanol saturated with HCl to yield the title intermediate as the hydrochloride salt.

### EXAMPLE 380

### Synthesis of N-(2-Chloro-5-nitropyrimidin-4-yl)-L-4-(N,N-dimethylcarbamyloxy)phenylalanine

### Step A—Preparation of 2,4-Dichloro-5-nitropyrimidine

5-Nitrouracil (Aldrich Chemical Company) was treated with phosphorous oxychloride and N,N-dimethylaniline according to the procedure described in Whittaker, J. Chem. Soc. 1951, 1565, to give 2,4-dichloro-5-nitropyrimidine as an orange oil which was used without distillation immediately in the next step.

### Step B—Preparation of N-(2-Chloro-5-nitropyrimidin-4-yl)-L-4-(N,N-dimethylcarbamyloxy)phenylalanine tert-Butyl Ester

To a stirred solution of L-4-(N,N-dimethylcarbamyloxy)phenylalanine tert-butyl ester (6.38 g, 2069 mol) and N,N-diisopropylethylamine (5.40 mL, 4.01 g, 31.03 mol.) in 70 mL CH₂Cl₂ at 0° C., was added a solution of 2,4-dichloro-5-nitropyrimidine (3.25 g, 20.69 mol.) in 70 mL CH₂Cl₂ at such a rate that the temperature did not exceed 10° C. After the addition, the mixture was stirred at 0-110° C. for 15 minutes, at which point TLC indicated conversion of the starting materials. To the mixture were added 100 mL 1 M KHSO₄ and 200 mL diethyl ether. The organic layer was separated, washed (H₂O, sat. NaHCO₃, and sat. NaCl), dried (MgSO₄), filtered, and evaporated to give the title compound (9.52 g, 2045 mol., 99%) as an orange oil.

### Step C—Preparation of N-(2-Chloro-5-nitropyrimidin-4-yl)-L-4-(N,N-dimethylcarbamyloxy)phenylalanine

The title compound was prepared by hydrolysis of the product from Step B using the procedure of Example 384.

### EXAMPLE 381

### Synthesis of N-[5-(N-4-Toluenesulfonylamino)pyrimidin-4-yl]-L-4-(N,N-dimethylcarbamyloxy)phenylalanine tert-Butyl Ester

### Step A—Preparation of N-(5-Aminopyrimidin-4-yl)-L-4-(N,N-dimethylcarbamyloxy)phenylalanine tert-Butyl Ester

A mixture of N-(2-chloro-5-nitropyrimidin-4-yl)-L-4-(N,N-dimethylcarbamyloxy)phenylalanine tert-butyl ester (9.52 g, 20.45 mol), Degussa-type 20% palladium on carbon (9.52 g), NaHCO₃ (8.59 g, 102.2 mol), and 165 mL MeOH was shaken under 55 psi for 16 h, at which point TLC indicated conversion of the starting material into a single product. The mixture was filtered through Celite, and the filtrate was evaporated to give a residue, which was dissolved by addition of 150 mL EtOAc and 75 mL H₂O. The organic layer was separated, washed (sat. NaCl), dried (MgSO₄), filtered, and evaporated to give the title intermediate (7.14 g, 17.79 mol, 87%) as an orange solid, which was used immediately in the next step.

### Step B—Preparation of N-[5-(N-4-Toluenesulfonyl-amino)pyrimidin-4-yl]-L-4-(N,N-dimethylcarbamyloxy)phenylalanine tert-Butyl Ester

To a stirred solution of the product from Step A (100 g, 2.49 mol) in 10 mL anhydrous pyridine at 0° C., was added in portions 4-toluenesulfonyl chloride (0.474 g, 2.49 mol). After the addition, the resulting red solution was stirred at 0° C. for 3 h, at which point TLC indicated nearly complete conversion of the starting material. To the mixture was added 3-dimethylaminopropylamine (0.325 mL, 0.264 g, 2.49 mol), and the mixture was stirred for 30 min while warming to room temperature. The mixture was poured into 100 mL 1 M KHSO₄, and extracted with 150 mL EtOAc. The organic layer was washed (2×1 M KHSO₄, H₂O, sat. NaHCO₃, sat. NaCl), dried (MgSO₄), filtered, and evaporated to give a brown residue, which was purified by flash chromatography using EtOAc/hexanes on silica gel, to give the title compound (1.01 g, 1.81 mol., 73%) as a clear oil.

### EXAMPLE 382

### Synthesis of N-[5-(N-4-Toluenesulfonylamino)pyrimidin-4-yl]-L-4-(N,N-dimethylcarbamyloxy)phenylalanine

The title compound was prepared by hydrolysis of N-[5-(N-4-toluenesulfonylamino)pyrimidin-4-yl]-L-4-(N,N-dimethylcarbamyloxy)-phenylalanine tert-butyl ester using the procedure of Example 384.

### EXAMPLE 383

### Synthesis of N-[5-(N-Methyl-N-4-toluenesulfonylamino)pyrimidin-4-yl]-L-4-(N,N-dimethylcarbamyloxy)phenylalanine tert-Butyl Ester

To a stirred two-phase mixture of 45 mL 1 M NaOH and 25 mL diethyl ether at 0° C., was added in portions 1-methyl-3-nitro-1-nitrosoguanidine (1.33 g, 9.05 mol). After stirring for 25 min, at which point evolution of N₂ had subsided, the bright yellow solution of diazomethane in diethyl ether was transferred by pipette to a stirred solution of the product of Example 381 (1.01 g, 1.81 mol) in 15 mL diethyl ether and 15 mL CH₂Cl₂ at 0° C. After stirring for 15 min, at which point TLC indicated complete conversion of the starting material, excess AcOH was added to destroy unreacted diazomethane. The mixture was diluted with 100 mL diethyl ether, washed (2×sat. NaHCO₃, sat. NaCl), dried (MgSO₄), filtered and evaporated to give a yellow residue, which was purified by flash chromatography using EtOAc/hexanes on silica gel, to give the title compound (0.846 g, 1.48 mol, 82%) as a clear oil.

### EXAMPLE 384

### Synthesis of N-[5-(N-Methyl-N-4-toluenesulfonylamino)pyrimidin-4-yl]-L-4-(N,N-dimethylcarbamyloxy)phenylalanine

The product of Example 383 (0.400 g, 0.700 mol) was dissolved in 8 mL 96% formic acid, and the mixture was heated to 40° C. for 16 h, at which point TLC indicated conversion of the starting material. Most of the formic acid was evaporated under a stream of N₂, and then the residue was placed under high vacuum for 48 h to give the title compound (0.382 g, 0.700 mol, 100%) as a clear oil.

Physical data were as follows:

¹H NMR (CD₃OD): δ=8.33 (bs, 1H), 8.07 (bs, 1H), 7.64 (d, J=8.1 Hz, 2H), 7.42 (d, J=8.1 Hz, 2H), 7.36 (bs, 1H), 7.29 (bs, 2H), 6.99 (d, J=7.5 Hz, 2H), 5.07-4.96 (m, 1H), 3.42-3.31 (m, 1H), 3.25-3.15 (m, 1H), 3.08 (s, 3H), 3.05 (bs, 3H), 2.96 (s, 3H), 2.44 (s, 3H).

¹³C NMR (CD₃OD): δ=174.7, 174.6, 164.6, 157.8, 156.8, 152.9, 152.1, 146.5, 135.4, 135.1, 131.7, 131.3, 129.4, 123.2, 122.9, 55.8, 38.2, 37.1, 36.8, 36.7, 21.5.

Using the appropriate starting materials and reagents, the following additional compounds were prepared:
N-[5-(N,N-Di-4-toluenesulfonylamino)pyrimidin-4-yl]-L-4-(N,N-dimethylcarbamyloxy)phenylalanine (Example 385);
N-[5-[N-(1-N′-Methylpyrazol-4-ylsulfonyl)-N-methylamino]pyrimidin-4-yl]-L-4-(N,N-dimethylcarbamyloxy)phenylalanine (Example 386);
N-[5-(N-Methyl-N-4-toluenesulfonylamino)pyrimidin-4-yl]-L-4-(N,N-dimethylcarbamyloxy)phenylalanine isopropyl ester (Example 387);
N-[5-(N-Methyl-N-3-pyridylsulfonylamino)pyrimidin-4-yl]-L-4-(N,N-dimethylcarbamyloxy)phenylalanine tert-butyl ester (Example 388).

### EXAMPLE 389

### Synthesis of N-(5-(N-Methyl-N-(1-butylpyrazol-4-yl)sulfonylamino)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 5-Nitrouracil (Aldrich) was converted via Method AAA into 2,4-dichloro-5-nitropyrimidine. L-4-(N,N-Dimethylcarbamyloxy)-phenylalanine tert-butyl ester and 2,4-dichloro-5-nitropyrimidine were coupled via Method BBB, and the product of this coupling was sequentially converted via Methods CCC, DDD (using 1-butyl-4-chlorosulfonylpyrazole), EEE and ZZ to give the title compound.

Physical data were as follows:

¹H NMR (CD₃OD): δ=8.35 (s, 1H), 8.14 (s, 1H), 7.76 (s, 1H), 7.61 (bs, 1H), 7.23 (bs, 2H), 6.98 (d, 2H), 5.01-4.94 (m, 1H), 4.19 (t, 2H), 3.40-3.28 (m, 1H), 3.26-3.14 (m, 1H), 3.09 (s, 3H), 3.06 (bs, 3H), 2.96 (s, 3H), 1.84 (pent., 2H), 1.29 (sext., 2H), 0.945 (t, 3H).

### EXAMPLE 390

### Synthesis of N-(5-(2,4-Dimethoxypyrimidin-5-yl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-pyrimidinone (Aldrich) was sequentially converted via Methods N and O into 4-chloro-5-iodopyrimidine. L-4-(N,N-dimethylcarbamyloxy)phenylalanine tert-butyl ester and 4-chloro-5-iodopyrimidine were coupled via Method P, and the coupled product was reacted with 2,4-dimethoxypyrimidin-5-yl boronic acid (Frontier Scientific, Inc.) via Method S. The product of this coupling was converted via Method KK to give the title compound.

### EXAMPLE 391

### Synthesis of N-(5-(2,6-Difluorophenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P, and the coupled product was reacted with 2,6-difluorophenyl boronic acid (Lancaster Synthesis) via Method R. The product of this coupling was converted via Method HH to give the title compound.

### EXAMPLE 392

### Synthesis of N-(5-(2-Hydroxymethylphenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-dimethylcarbamyloxy)phenylalanine tert-butyl ester and 4-chloro-5-iodopyrimidine were coupled via Method P, and the coupled product was reacted with 2-(hydroxymethyl)phenyl boronic acid (Lancaster Synthesis) via Method Q. The product of this coupling was converted via Method HH to give the title compound.

### EXAMPLE 393

### Synthesis of N-(2-(N-Cyclohexylamino)-5-(2-tolyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with cyclohexylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =9.68 (s, 1H), 7.3-6.8 (m, 9H), 6.35 (m, 1H), 4.73 (m, 1H), 3.81 (bs, 1H), 3.6-3.0 (m, 2H), 3.09 (s, 3H), 3.0 (s, 3H), 2.18 (s, 1.5H), 1.94 (s, 1.5H), 2.1-1.1 (m, 10H).

¹³C NMR (CDCl₃): δ =176.11, 175.94, 160.05, 159.79, 154.76, 153.58, 150.05, 150.01, 139.26, 137.84, 137.63, 134.29, 134.15, 130.66, 130.36, 130.11, 129.14, 126.70, 126.41, 121.25, 109.57, 109.39, 56.84, 56.35, 50.15, 36.55, 36.32, 32.34, 31.99, 25.41, 24.86, 19.48, 19.27.

### EXAMPLE 394

### Synthesis of N-(2-(N-Methyl-N-(1-methylpiperidin-4-yl)amino)-5-(2-tolyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with 1-methyl-4-(N-methylamino)piperidine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =8.82 (s, 2H), 8.43 (s, 1H), 7.62 (s, 1H), 7.30-6.90 (m, 8H), 5.42 (br, 1H), 4.66 (br, 2H), 3.60-2.8 (m, 15H), 2.66 (bs, 3H), 2.32 (br, 2H), 2.18 (s, 1.5H), 1.82 (brs, 3.5H).

### EXAMPLE 395

### Synthesis of N-(2-(N-Ethyl-N-isopropylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-ethyl-N-isopropylamine (Aldrich) via Method CC to give a product that was coupled with o-tolyl boronic acid (Aldrich) via Method DD.

The product of this coupling was converted via Method ZZ to give the title compound.

Physical data were as follows:

¹H NMR (CDCl₃): δ =8.0-6.5 (br, 1H), 7.66 (s, 0.5H), 7.62 (s, 0.5H), 7.3-6.8 (m, 8H), 6.2 (m, 1H), 4.86 (br, 1H), 4.70 (m, 1H), 3.70-3.08 (m, 4H), 3.09 (s, 3H), 3.0 (s, 3H), 2.14 (bs, 1.5H), 1.92 (bs, 1.5H), 1.4-0.9 (br, 9H).

¹³C NMR (CDCl₃): δ =174.38, 174.19, 159.44, 159.16, 155.24, 154.68, 152.39, 150.02, 141.63, 137.77, 137.56, 134.30, 134.09, 130.79, 130.66, 130.54, 130.46, 130.41, 130.33, 130.08, 129.07, 126.54, 126.45, 126.38, 121.21, 121.16, 110.27, 110.01, 56.77, 56.36, 47.59, 36.80, 36.55, 36.32, 20.27, 20.18, 19.57, 19.38, 14.51.

### EXAMPLE 396

### Synthesis of N-(5-(2,4-6-Trimethylphenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P, and the coupled product was reacted with 2,4,6-trimethylphenyl boronic acid (Frontier Scientific, Inc) via Method R. The product of this coupling was converted via Method HH to give the title compound.

Physical data were as follows:

¹H NMR (CD₃OD): δ=8.68 (d, 1H), 7.95 (d, 1H), 7.10 (d, 2H), 7.09-6.95 (m, 2H), 6.94-6.91 (m, 2H), 5.32-5.27 (m, 1H), 3.42-3.36 (m, 1H), 3.15-3.09 (m, 4H), 2.97 (s, 3H), 2.33 (s, 3H), 2.04 (s, 3H), 1.84 (s, 3H).

¹³C NMR (CD₃OD): δ=172.9, 163.5, 161.5, 161.0, 156.7, 152.0, 151.9, 142.6, 141.5, 138.9, 138.6, 135.3, 131.2, 130.4, 130.3, 126.5, 123.0, 120.3, 56.4, 36.7, 36.6, 36.5, 21.2, 19.9, 19.7.

### EXAMPLE 397

### Synthesis of N-(5-Isopropylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. Diethyl 2-isopropylmalonate (Aldrich) was sequentially converted via Methods OO and QQ into 4,6-dichloro-5-isopropylpyrimidine. L-4-(N,N-Dimethylcarbamyloxy)phenylalanine tert-butyl ester and 4,6-dichloro-5-isopropylpyrimidine were coupled via Method RR, and the product of this coupling was sequentially converted via Methods SS and ZZ to give the title compound.

Physical data were as follows:

¹H NMR (CD₃OD): δ=8.44 (bs, 1H), 7.94 (bs, 1H), 7.22 (d, 2H), 6.94 (d, 2H), 5.12 (dd, 1H), 3.46 (dd, 1H), 3.19 (dd, 1H), 3.07 (s, 3H), 2.95 (s, 3H), 3.00-2.88 (m, 1H), 1.25 (d, 3H), 1.13 (d, 3H).

¹³C NMR (CD₃OD): δ=175.60, 165.74, 163.78, 156.91, 152.38, 151.85, 141.88, 136.30, 131.43, 126.17, 122.87, 57.84, 37.48, 36.81, 36.64, 26.63, 21.09, 20.94.

### EXAMPLE 398

### Synthesis of N-(2-(N-Methyl-N-butylamino)-5-(2-tolyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-butylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =12.5-11.4 (br, 1H), 7.6 (s, 0.5H), 7.58 (s, 0.5H), 7.3-6.8 (m, 8H), 6.3 (m, 1H), 4.7 (m, 1H), 3.7-2.9 (m, 4H), 3.08 (s, 3H), 3.01 (s, 6H), 2.13 (s, 1.5H), 1.91 (s, 1.5H), 1.57 (bs, 2H), 1.33 (m, 2H), 0.96 (t, 3H).

¹³C NMR (CDCl₃): δ =174.21, 174.06, 159.37, 159.22, 154.69, 153.52, 169.99, 141.87, 137.77, 137.54, 134.43, 130.78, 130.59, 130.10, 128.98, 126.51, 126.32, 121.17, 121.11, 110.20, 109.96, 56.82, 56.43, 50.03, 36.54, 36.32, 35.91, 29.27, 19.89, 19.52, 19.35, 13.84.

### EXAMPLE 399

### Synthesis of N-(2-(N-Ethyl-N-propylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-ethyl-N-propylamine (Aldrich) via Method CC to give a product that was coupled with o-tolyl boronic acid (Aldrich) via Method DD. The product of this coupling was converted via Method ZZ to give the title compound.

Physical data were as follows:

1H NMR (CDCl₃): δ =11.0-9.5 (br, 1H), 7.66 (s, 0.5H), 7.64 (s, 0.5H), 7.4-6.8 (m, 8H), 6.28 (m, 1H), 4.65 (m, 1H), 3.70-2.80 (m, 6H), 3.09 (s, 3H), 3.01 (s, 3H), 3.01 (s, 3H), 2.2 (s, 1.5H), 1.85 (s, 1.5H), 1.58 (bs, 2H), 1.05 (bs, 3H), 0.85 (bs, 3H).

¹³C NMR (CDCl₃): δ =174.26, 174.11, 159.36, 159.11, 154.70, 153.07, 149.96, 142.43, 137.80, 137.56, 134.54, 134.37, 130.84, 130.74, 130.57, 130.14, 128.86, 126.47, 126.29, 121.10, 121.06, 110.01, 109.71, 56.86, 56.49, 49.62, 63.20, 36.55, 36.32, 20.87, 19.61, 19.41, 12.63, 11.03.

### EXAMPLE 400

### Synthesis of N-(2-(N,N-Diethylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N,N-diethylamine (Aldrich) via Method CC to give a product that was coupled with o-tolyl boronic acid (Aldrich) via Method DD. The product of this coupling was converted via Method ZZ to give the title compound.

Physical data were as follows:

H NMR (CDCl₃): δ =12.2 (br, 1H), 7.63 (s, 0.5H), 7.60 (s, 0.5H), 7.40-6.80 (m, 8H), 6.28 (m, 1H), 4.70 (m, 1H), 3.80-2.90 (m, 6H), 3.06 (s, 3H), 2.98 (s, 3H), 2.13 (s, 1.5H), 1.92 (s, 1.5H), 0.90 (s, 6H).

¹³C NMR (CDCl₃): δ =174.34, 174.15, 159.4, 159.1, 154.70, 152.66, 169.97, 142.06, 137.76, 137.55, 134.44, 134.27, 130.81, 130.57, 130.10, 128.95, 126.48, 126.32, 121.14, 121.08, 110.08, 109.80, 56.78, 56.37, 42.77, 36.53, 36.31, 19.57, 19.38, 12.77.

### EXAMPLE 401

### Synthesis of N-(2-(N-Methyl-N-ethylamino)-5-(2-tolyl)pyrimidin-4-yl)-L-4-(N,N-dimethylcarbamyloxy)phenylalanine

Tyrosine tert-butyl ester (Bachem) was sequentially converted via Methods U, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-ethylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =12.5 (br, 2H), 8.23 (s, 1H), 7.50 (s, 0.5H), 7.44 (s, 0.5H), 7.30-6.80 (m, 8H), 6.10 (m, 1H), 4.75 (m, 1H), 3.58 (bs, 2H), 3.30 (m, 1H), 3.00 (m, 1H), 3.08 (s, 3H), 3.00 (s, 3H), 2.93 (s, 3H), 2.08 (s, 1.5H), 1.92 (s, 1.5H), 1.50 (s, 3H).

¹³C NMR (CDCl₃): δ =174.63, 174.34, 165.72, 159.96, 159.72, 154.88, 152.62, 150.49, 150.45, 140.64, 137.90, 137.81, 133.83, 133.65, 131.03, 130.95, 130.85, 130.63, 130.10, 130.04, 129.76, 129.62, 126.88, 126.72, 121.70, 121.61, 110.69, 110.46, 56.65, 56.11, 45.16, 36.57, 36.35, 35.17, 19.38, 19.17, 11.96.

### EXAMPLE 402

### Synthesis of N-(5-Benzyloxypyrimidin-4-yl)-L-phenylalanine

Methyl 2-benzyloxyacetate (Aldrich) was sequentially converted via Methods PP and QQ into 4-chloro-5-benzyloxypyrimidine. L-4-phenylalanine tert-butyl ester (Bachem) and 4-chloro-5-benzyloxypyrimidine were coupled via Method RR, and the product of this coupling was converted via Method ZZ to give the title compound.

Physical data were as follows:

¹H NMR (CD₃OD): δ=8.54 (s, formate), 8.03 (s, 1H), 7.67 (s, 1H), 7.37-7.31 (m, 5H), 7.17-7.12 (m, 5H), 5.11 (s, 2H), 4.78-4.75 (m, 1H), 3.35-3.11 (m, 2H).

¹³C NMR (CD₃OD): δ=159.07, 143.16, 132.35, 130.64, 124.52, 123.94, 123.83, 123.59, 123.11, 122.00, 99.47, 66.28, 50.32, 32.05.

### EXAMPLE 403

### Synthesis of N-(5-Benzyloxypyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. Methyl 2-benzyloxyacetate (Aldrich) was sequentially converted via Methods PP and QQ into 4-chloro-5-benzyloxypyrimidine. L-4-(N,N-Dimethylcarbamyloxy)phenylalanine tert-butyl ester and 4-chloro-5-benzyloxypyrimidine were coupled via Method RR, and the product of this coupling was converted via Method ZZ to give the title compound.

### EXAMPLE 404

### Synthesis of N-(5-(N-Methyl-N-4-toluenesulfonylamino)pyrimidin-4-yl)-L-phenylalanine

5-Nitrouracil (Aldrich) was converted via Method AAA into 2,4-dichloro-5-nitropyrimidine. L-4-Phenylalanine tert-butyl ester (Bachem) and 2,4-dichloro-5-nitropyrimidine were coupled via Method BBB, and the product of this coupling was sequentially converted via Methods CCC, DDD, EEE and ZZ to give the title compound.

### EXAMPLE 405

### Synthesis of N-(5-(N-Methyl-N-3-pyridinesulfonylamino)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 5-Nitrouracil (Aldrich) was converted via Method AAA into 2,4-dichloro-5-nitropyrimidine. L-4-(N,N-Dimethylcarbamyloxy)-phenylalanine tert-butyl ester and 2,4-dichloro-5-nitropyrimidine were coupled via Method BBB, and the product of this coupling was sequentially converted via Methods CCC, DDD (using 3-chlorosulfonylpyridine), EEE and ZZ to give the title compound.

Physical data were as follows:

¹H NMR (CD₃OD): δ=8.90 (d, 1H), 8.85 (d, 1H), 8.36 (s, 1H), 8.15 (d, 1H), 7.64 (dd, 1H), 7.53 (bs, 1H), 7.27 (bs, 2H), 6.99 (d, 2H), 5.04-4.87 (m, 1H), 3.40-3.28 (m, 1H), 3.26-3.16 (m, 1H), 3.13 (bs, 3H), 3.09 (s, 3H), 2.97 (s, 3H).

### EXAMPLE 406

### Synthesis of N-(5-Phenylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P, and the coupled product was reacted with phenyl boronic acid (Aldrich) via Method S. The product of this coupling was converted via Method HH to give the title compound.

Physical data were as follows:

¹H NMR (CD₃OD): δ=8.62 (s, 1H), 8.04 (s, 1H), 7.53-7.51 (m, 3H), 7.30-7.27 (m, 2H), 7.17-7.15 (m, 2H), 7.00-6.97 (m, 2H), 5.27-5.22 (m, 1H), 3.45-3.39 (m, 1H), 3.16-3.08 (m, 4H), 2.96 (s, 3H).

¹³C NMR (CD₃OD): δ=173.8, 163.7, 157.5, 152.8, 152.3, 142.4, 135.9, 132.2, 132.1, 131.8, 130.7, 123.9, 122.4, 57.7, 37.7, 37.5.

### EXAMPLE 407

### Synthesis of N-(3-(N-Methyl-N-4-toluenesulfonylamino)pyrazin-2-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 2,3-Dichloropyrazine (Lancaster) was converted via Method QQQ and RRR into 2-chloro-3-nitropyrazine. L-4-(N,N-Dimethylcarbamyloxy)phenylalanine tert-butyl ester and 2-chloro-3-nitropyrazine were coupled via Method BBB, and the product of this coupling was sequentially converted via Methods CCC, DDD, EEE and ZZ to give the title compound.

Physical data were as follows:

¹H NMR (CD₃OD): δ=8.07 (s, formate), 7.94 (d, 1H), 7.59 (d, 2H), 7.51 (d, 1H), 7.36 (d, 2H), 7.29 (d, 2H), 7.01 (d, 2H), 4.90 (m, 1H), 3.30-3.18 (m, 2H), 3.08 (s, 3H), 2.96 (s, 3H), 2.94 (s, 3H), 2.43 (s, 3H).

¹³C NMR (CD₃OD): δ=177.07, 169.41, 158.64, 150.92, 147.23, 145.92, 139.97, 137.14, 133.12, 129.62, 128.90, 125.69, 124.67, 124.08, 116.86, 49.99, 31.67, 31.28, 30.77, 30.62, 15.46.

### EXAMPLE 408

### Synthesis of N-(5-(2,2,2-Trifluoroethyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 1-Trifluoromethanesulfonyloxy-2,2,2-trifluoroethane was sequentially converted via Methods LL, OO and QQ into 4,6-dichloro-5-(2,2,2-trifluoroethyl)pyrimidine. L-4-(N,N-Dimethylcarbamyloxy)-phenylalanine tert-butyl ester and 4,6-dichloro-5-(2,2,2-trifluoroethyl)pyrimidine were coupled via Method RR, and the product of this coupling was sequentially converted via Methods SS and ZZ to give the title compound.

Physical data were as follows:

1H NMR (CD₃OD): δ=8.41 (s, 1H), 8.09 (s, formate), 8.06 (s, 1H), 7.24 (d, 2H), 6.96 (d, 2H), 5.06 (m, 1H), 3.60-3.40 (m, 2H), 3.37-3.11 (m, 2H), 3.08 (s, 3H), 2.96 (s, 3H).

¹³C NMR (CD₃OD): δ=169.35, 158.91, 156.43, 151.33, 150.97, 148.87, 145.76, 130.21, 125.27, 116.80, 50.80, 31.34, 30.75, 30.60, 26.65, 26.23.

### EXAMPLE 409

### Synthesis of N-(5-(N-Methyl-N-3-pyridinesulfonylamino)pyrimidin-4-yl)-L-4-(4-methylpiperazin-1-ylcarbonyloxy)phenylalanine Isopropyl Ester

L-Tyrosine (Aldrich) was sequentially converted via Methods LLL, UU, XX and YY into L-4-(4-methylpiperazin-1-ylcarbonyloxy)phenylalanine isopropyl ester. 5-Nitrouracil (Aldrich) was converted via Method AAA into 2,4-dichloro-5-nitropyrimidine. L-4-(4-Methylpiperazin-1-ylcarbonyloxy)phenylalanine isopropyl ester and 2,4-dichloro-5-nitropyrimidine were coupled via Method BBB, and the product of this coupling was sequentially converted via Methods CCC, DDD (using 3-chlorosulfonylpyridine) and EEE to give the title compound.

### EXAMPLE 410

### Synthesis of N-(5-Benzylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. Diethyl 2-benzylmalonate (Aldrich) was sequentially converted via Methods OO and QQ into 4,6-dichloro-5-benzylpyrimidine. L-4-(N,N-Dimethylcarbamyloxy)phenylalanine tert-butyl ester and 4,6-dichloro-5-benzylpyrimidine were coupled via Method RR, and the product of this coupling was sequentially converted via Methods SS and ZZ to give the title compound.

Physical data were as follows:

¹H NMR (CD₃OD): δ=8.41 (s, 1H), 8.13 (s, formate), 7.80 (s, 1H) 7.34-7.19 (m, 3H), 7.17 (d, 2H), 7.00 (d, 2H), 6.85 (d, 2H), 5.01 (m, 1H), 3.82 (m, 2H), 3.09 (s, 3H), 3.09-2.97 (m, 2H), 2.97 (s, 3H).

¹³C NMR (CD₃OD): δ=159.31, 156.23, 150.88, 148.07, 145.70, 141.38, 131.56, 129.81, 125.30, 124.21, 124.01, 122.37, 116.81, 51.35, 31.68, 30.78, 30.61, 28.28.

### EXAMPLE 411

### Synthesis of N-(5-(N-Methyl-N-3-pyridinesulfonylamino)pyrimidin-4-yl)-L-4-(4-methylpiperazin-1-ylcarbonyloxy)phenylalanine tert-Butyl Ester

L-Tyrosine tert-butyl ester (Bachem) was sequentially converted via Methods UU, XX and YY into L-4-(4-methylpiperazin-1-ylcarbonyloxy)phenylalanine tert-butyl ester. 5-Nitrouracil (Aldrich) was converted via Method AAA into 2,4-dichloro-5-nitropyrimidine. L-4-(4-Methylpiperazin-1-ylcarbonyloxy)phenylalanine tert-butyl ester and 2,4-dichloro-5-nitropyrimidine were coupled via Method BBB, and the product of this coupling was sequentially converted via Methods CCC, DDD (using 3-chlorosulfonylpyridine) and EEE to give the title compound.

### EXAMPLE 412

### Synthesis of N-(5-(2-Trifluoromethylphenyl)pyrimidin-4-yl)-L-4-(N,N-dimethylcarbamyloxy)phenylalanine

L-Tyrosine tert-butyl ester (Bachem) was sequentially converted via UU, VV and WW into L-4-(N,N-dimethylcarbamyloxy)phenylalanine tert-butyl ester. 4(3H)-Pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with 2-trifluoromethylphenyl boronic acid (Aldrich) via Method Q. The product of this coupling was converted via Method HH to give the title compound.

Physical data were as follows:

¹H NMR (CD₃OD): δ=8.51 (s, 1H), 7.84-7.49 (m, 2H), 7.71-7.63 (m, 2H), 7.37 (d, 1H), 7.11-6.97 (m, 4H), 6.88 (d, 1H), 4.99 (s, 1H), 3.37-3.19 (m, 1H), 3.14-3.02 (m, 4H), 2.97 (s, 3H).

¹³C NMR (CD₃OD): δ=175.7, 175.5, 165.6, 161.9, 161.7, 158.6, 157.6, 157.5, 153.3, 153.1, 152.6, 152.5, 136.4, 136.2, 135.0, 134.9, 134.5, 133.1, 132.2, 131.9, 131.7, 128.9, 128.7, 127.8, 124.3, 123.6.

### EXAMPLE 413

### Synthesis of N-(5-(2-N,N-Dimethylcarbamylethyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with dimethylacrylamide (Aldrich) via Method U. The product of this reaction which was sequentially converted via Methods V and HH to give the title compound.

Physical data were as follows:

¹H NMR (CD₃OD): δ=8.56 (s, 1H), 8.06 (s, 1H), 7.32 (d, 2H), 7.01 (d, 2H), 5.35-5.30 (m, 1H), 3.56-3.49 (m, 1H), 3.23-3.18 (m, 1H), 3.11 (s, 3H), 3.02 (s, 3H), 2.99 (s, 3H), 2.97 (s, 3H), 2.88 (t, 2H), 2.65 (t, 2H).

¹³C NMR (CD₃OD): δ=174.5, 174.2, 152.7, 151.6, 142.6, 136.5, 132.0, 123.8, 121.0, 57.8, 38.4, 37.9, 37.5, 36.9, 32.2, 24.6.

### EXAMPLE 414

### Synthesis of N-(5-(N-Methyl-N-3-(1-methylpyrazole)sulfonylamino)pyrimidin-4-yl)-L-4-(N,N-dimethylcarbamyloxy)phenylalanine Isopropyl Ester

L-Tyrosine (Aldrich) was sequentially converted via Methods LLL, UU, VV and WW into L-4-(N,N-dimethylcarbamyloxy)phenylalanine isopropyl ester. 5-Nitrouracil (Aldrich) was converted via Method AAA into 2,4-dichloro-5-nitropyrimidine. L-4-(N,N-Dimethylcarbamyloxy)-phenylalanine isopropyl ester and 2,4-dichloro-5-nitropyrimidine were coupled via Method BBB, and the product of this coupling was sequentially converted via Methods CCC, DDD (using 1-methyl-3-chlorosulfonylpyrazole) and EEE to give the title compound.

Physical data were as follows:

¹H NMR (CDCl₃) δ=8.47 (s, 1H), 7.76 (s, 1H), 7.68 (bs, 2H), 7.19 (m, 2H), 7.04 (d, 2H), 6.17 (d, 1H), 5.03 (m, 2H), 3.95 (s, 3H), 3.31-3.12 (m, 2H), 3.08 (s, 3H), 3.06 (s, 3H), 2.99 (s, 3H), 1.24 (d, 3H), 1.21 (d, 3H).

### EXAMPLE 415

### Synthesis of N-(6-Phenylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 4,6-dichloropyrimidine (Aldrich) were coupled via Method Y and the coupled product was reacted with phenyl boronic acid (Aldrich) via Method Z. The product of this coupling was converted via Method HH to give the title compound.

Physical data were as follows:

¹H NMR (CD₃OD): δ=8.65 (s, 1H), 7.82-7.79 (m, 2H), 7.77-7.62 (m, 3H), 7.31 (d, 2H), 7.06-7.01 (m, 4H), 5.32-5.28 (m, 1H), 3.50-3.44 (m, 1H), 3.20-3.06 (m, 4H), 2.99 (s, 3H).

¹³C NMR (CD₃OD): δ=173.9, 165.7, 157.6, 154.9, 154.3, 152.8, 135.8, 134.6, 132.3, 132.2, 131.7, 129.2, 123.8, 104.6, 57.8, 38.8, 37.7, 37.5.

### EXAMPLE 416

### Synthesis of N-(6-(2-Trifluoromethylphenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 4,6-dichloropyrimidine (Aldrich) were coupled via Method Y and the coupled product was reacted with 2-trifluoromethylphenyl boronic acid (Aldrich) via Method Z. The product of this coupling was converted via Method HH to give the title compound.

Physical data were as follows:

¹H NMR (CD₃OD): δ=8.46 (s, 1H), 7.95-7.82 (m, 1H), 7.73-7.67 (m, 2H), 7.50-7.48 (m, 1H), 7.29 (d, 2H), 7.03 (d, 2H), 6.65 (s, 1H), 5.05 (s, 1H), 3.39 (m, 1H), 3.16-3.12 (m, 4H), 3.00 (s, 3H).

¹³C NMR (CD₃OD): δ=176.0, 164.3, 158.8, 157.7, 152.6, 136.6, 139.0, 132.9, 132.1, 131.4, 130.1, 129.7, 128.2, 128.2, 123.6, 38.8, 37.7, 37.5.

### EXAMPLE 417

### Synthesis of N-(6-(2-Hydroxymethylphenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbarnyloxy)-phenylalanine tert-butyl ester. L-4-(N,N-Dimethylcarbarnyloxy)-phenylalanine tert-butyl ester and 4,6-dichloropyrimidine (Aldrich) were coupled via Method Y and the coupled product was reacted with 2-(hydroxymethyl)phenyl boronic acid (Lancaster Synthesis) via Method Z. The product of this coupling was converted via Method HH to give the title compound.

Physical data were as follows:

¹H NMR (CD₃OD): δ=8.48 (s, 1H), 8.09 (s, 1H), 7.61-7.44 (m, 4H), 7.29 (d, 2H), 7.02 (d, 2H), 6.71 (s, 1H), 5.27 (s, 2H), 5.10-5.02 (m, 1H), 3.42-3.41 (m, 1H), 3.16-3.12 (m, 4H), 2.99 (s, 3H).

¹³C NMR (CD₃OD): δ=175.7, 165.6, 164.7, 158.0, 157.6, 152.6, 141.6, 138.5, 136.7, 135.8, 132.2, 131.9, 131.7, 131.4, 131.3, 123.7, 64.9, 64.3, 38.9, 37.7, 37.5.

### EXAMPLE 418

### Synthesis of

### N-(5—Cyclohexylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. Cyclohexanone (Aldrich) was sequentially converted via Methods MM, NN, OO and QQ into 4,6-dichloro-5-cyclohexylpyrimidine. L-4-(N,N-Dimethylcarbamyloxy)phenylalanine tert-butyl ester and 4,6-dichloro-5-cyclohexylpyrimidine were coupled via Method RR, and the product of this coupling was sequentially converted via Methods SS and ZZ to give the title compound.

Physical data were as follows:

¹H NMR (CD₃OD): δ=8.41 (bs, 1H), 7.89 (bs, 1H), 7.21 (d, 2H), 6.94 (d, 2H), 5.12 (dd, 1H), 3.47 (dd, 1H), 3.19 (dd, 1H), 3.06 (s, 3H), 2.95 (s, 3H), 3.0 (m, 1H), 2.88-2.57 (bs, 1H), 2.5 (bs, 1H), 1.95-1.67 (m, 1H).

¹³C NMR (CD₃OD): δ=175.68, 165.82, 156.87, 152.10, 151.88, 141.96, 136.30, 131.44, 125.38, 122.89, 57.86, 37.44, 36.81, 36.64, 36.30, 32.65, 32.13, 27.29, 27.25, 26.95.

### EXAMPLE 419

### Synthesis of N-(2-(N-Methyl-N-2-furanmethylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methylfurfurylamine (Salor) via Method CC to give a product that was coupled with o-tolyl boronic acid (Aldrich) via Method DD. The product of this coupling was converted via Method ZZ to give the title compound.

Physical data were as follows:

¹H NMR (CD₃OD): δ=7.43-7.35 (m, 2H), 7.35-7.2 (m, 2H), 7.2-7.0 (m, 4H), 7.0-6.9 (m, 2H), 6.42 (d, 0.1H), 6.39 (d, 1H), 4.85 (m, 1H), 3.3-3.1 (m, 7H), 3.09 (s, 3H), 2.98 (s, 3H), 2.16 (s, 3H), 1.89 (s, 3H).

### EXAMPLE 420

### Synthesis of N-(2-(N-Methyl-N-4-chloropheylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-4-chloroaniline (Aldrich) via Method CC to give a product that was coupled with o-tolyl boronic acid (Aldrich) via Method DD. The product of this coupling was converted via Method ZZ to give the title compound.

Physical data were as follows:

¹H NMR (CD₃OD): δ=8.17 (s, 1H), 7.56-7.34 (m, 8H), 7.1-6.97 (m, 4H), 3.50 (m, 2H), 3.13 (s, 3H), 2.1 (s, 3H), 2.17 (s, 3H), 1.94 (s, 3H).

### EXAMPLE 421

### Synthesis of N-(5-(3-Thienyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with 3-thiophenyl boronic acid (Frontier Scientific, Inc.) via Method S. The product of this coupling was converted via Method KK to give the title compound.

Physical data were as follows:

¹H NMR (CD₃OD): δ=8.62 (s, 1H), 8.13 (s, 1H), 7.62 (m, 1H), 7.59 (m, 1H), 7.20 (d, 2H), 7.09 (d, 1H), 7.01 (d, 2H), 3.47-3.13 (m, 2H), 3.13 (s, 3H), 2.97 (s, 3H).

¹³C NMR: δ=173.22, 162.83, 156.84, 152.17, 151.43, 141.46, 135.22, 131.54, 131.35, 129.96, 127.99, 127.90, 123.24, 117.13, 56.87, 36.82, 36.64.

### EXAMPLE 422

### Synthesis of N-(5-(2-Thienyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O to 4-chloro-5-iodopyrimidine. L-4-(N,N-dimethylcarbamyloxy)phenylalanine tert-butyl ester and 4-chloro-5-iodopyrimidine were coupled via Method P, and the coupled product was reacted with 2-thiophenyl boronic acid (Frontier Scientific, Inc.) via Method S. The product of this coupling was converted via Method KK to give the title compound.

Physical data were as follows:

¹H NMR (CD₃OD): δ=8.10 (s, 1H), 7.67 (s, 1H), 7.19 (d, 1H), 6.73 (m, 4H), 6.49 (m, 2H), 4.80 (m, 1H), 2.89 (m, 1H), 2.70 (m, 1H), 2.60 (s, 3H), 2.45 (s, 3H).

¹³C NMR (CD₃OD): δ=173.07, 162.72, 156.80, 152.13, 151.74, 142.30, 135.07, 131.58, 131.14, 130.69, 130.38, 129.92, 123.19, 115.18, 56.94, 36.87, 36.81, 36.62, 28.74.

### EXAMPLE 423

### Synthesis of N-(2-(N-Methyl-N-2-hydroxyethylamino)-5-(2-fluorophenyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with 2-(N-methylamino)ethanol (Aldrich) via Method CC to give a product that was coupled with 2-fluorophenyl boronic acid (Aldrich) via Method DD. The product of this coupling was converted via Method KK to give the title compound.

### EXAMPLE 424

### Synthesis of N-(5-(Piperidin-1-yl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. Piperidine (Aldrich) was sequentially converted via Methods FFF, GGG and HHH into 4,6-dichloro-5-piperidin-1-ylpyrimidine. L-4-(N,N-Dimethylcarbamyloxy)phenylalanine tert-butyl ester and 4,6-dichloro-5-piperidin-1-ylpyrimidine were coupled via Method III, and the product of this coupling was sequentially converted via Methods JJJ and ZZ into the title compound.

### EXAMPLE 425

### Synthesis of N-(5-(1-Propylbutyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4-Heptanone (Aldrich) was sequentially converted via Methods MM, NN, 00 and QQ into 4,6-dichloro-5-(1-propylbutyl)pyrimidine. L-4-(N,N-Dimethylcarbamyloxy)phenylalanine tert-butyl ester and 4,6-dichloro-5-(1-propylbutyl)pyrimidine were coupled via Method RR, and the product of this coupling was sequentially converted via Methods SS and ZZ to give the title compound.

### EXAMPLE 426

### Synthesis of N-(2-(N-Methyl-N-cyclobutylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methylcyclobutylamine (prepared by the Method of Giardina et al. J. Med. Chem. 1994, 37(21), 3482-3491) via Method CC to give a product that was coupled with o-tolyl boronic acid (Aldrich) via Method DD. The product of this coupling was converted via Method ZZ to give the title compound.

### EXAMPLE 427

### Synthesis of N-(2-(N,N-Bis-(2-hydroxyethyl)amino)pyrimidin-4-yl)-L-4-(N,N-dimethylcarbamyloxy)phenylalanine

A byproduct was isolated by chromatography of the crude product of Example 428, and the byproduct was converted via Method KK into the title compound.

Physical data were as follows:

¹H NMR (CD₃OD): δ=7.59 (d, 1H), 7.25 (d, 2H), 7.02 (d, 2H), 6.18 (d, 1H), 3.76 (brs, 8H), 2.97 (s, 8H).

¹³C NMR (CD₃OD): δ=174.1, 163.7, 155, 152, 142.1, 135.2, 131.3, 123.7, 99, 60.5, 56.8, 53.2, 37.5, 36.8, 36.6.

### EXAMPLE 428

### Synthesis of N-(2-(N,N-bis-(2-Hydroxyethylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with diethanolamine (Aldrich) via Method CC to give a product that was coupled with o-tolyl boronic acid (Aldrich) via Method DD. The product of this coupling was converted via Method KK to give the title compound.

Physical data were as follows:

H NMR (CD₃OD): δ=7.48-7.31 (m, 5H), 7.15-6.98 (m, 4H), 4.9 (m, 1H), 4.63 (m, 1H), 3.83 (d, 8H), 3.1 (s, 8H), 1.9 (d, 3H).

¹³C NMR (CD₃OD): δ=173.8, 162.3, 154.6, 152.6, 140.9, 139.6, 139.4, 135.9, 135.8, 132.2, 132.0, 131.4, 131.2, 131.1, 128, 123.2, 123.1, 66.8, 60.6, 56.9, 56.4, 53.2, 52.8, 36.8, 36.6, 36.3, 19.5.

### EXAMPLE 429

### Synthesis of N-(2-(N-Methyl-N-phenylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methylaniline (Aldrich) via Method CC to give a product that was coupled with o-tolyl boronic acid (Aldrich) via Method DD. The product of this coupling was converted via Method ZZ to give the title compound.

Physical data were as follows:

¹H NMR (CD₃OD): δ=7.57-6.99 (m, 14H), 4.99 (m, 1H), 3.49 (s, 3H), 3.11 (m, 5H), 2.98 (s, 3H), 2.16 (s, 3H).

¹³C NMR (CD₃OD): δ=183.07, 173.72, 173.49, 162.55, 156.82, 153.97, 152.07, 142.25, 141.06, 140.91, 139.53, 139.40, 135.50, 135.39, 132.21, 132.16, 132.05, 131.52, 131.31, 130.53, 128.44, 128.11, 128.00, 123.13, 123.04, 113.18, 56.95, 56.49, 40.02, 39.96, 37.14, 36.83, 36.65, 19.56, 19.47.

### EXAMPLE 430

### Synthesis of N-(2-(Isopropoxy)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this coupling was sequentially converted via Methods NNN, DD (using o-tolyl boronic acid, Aldrich) and ZZ to give the title compound.

Physical data were as follows:

¹H NMR (CDCl₃): δ =7.77 (bs, 1H), 7.40-6.8 (m, 9H), 6.43 (d, 0.5H) 6.27 (d, 0.5H), 6.78 (m, 1H), 6.16 (m, 1H), 3.09 (s, 3H), 3.00 (s, 3H), 3.40-2.80 (m, 4H), 2.20 (s, 1.5H), 1.94 (s, 1.5H), 1.23 (m, 6H).

¹³C NMR (CDCl₃): δ =176.28, 176.15, 160.03, 159.78, 154.77, 153.65, 150.01, 169.97, 139.20, 137.81, 137.64, 134.39, 134.25, 130.71, 130.47, 130.12, 129.15, 126.69, 126.46, 121.24, 121.18, 109.56, 56.81, 56.34, 63.19, 36.90, 36.56, 36.32, 22.19, 21.99, 21.95, 19.51, 19.27.

### EXAMPLE 431

### Synthesis of N-(2-(N-Methyl-N-3-methylbutylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methylN-isoamylamine (Pfaltz-Bauer) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =7.6 (s, 0.5H), 7.56 (s, 0.5H), 7.30-6.80 (m, 8H) 6.30 (bm, 1H), 7.00-6.00 (br, 1H), 4.63 (m, 1H), 3.09 (s, 3H), 3.01 (s, 6H), 3.80-2.80 (m, 4H), 2.13 (s, 1.5H), 1.90 (s, 1.5H), 1.61 (m, 1H), 1.51 (bs, 2H), 0.96 (d, 6H).

¹³C NMR (CDCl₃): δ =174.03, 173.87, 159.28, 159.04, 154.71, 153.67, 150.00, 142.10, 137.81, 137.53, 134.39, 134.22, 130.78, 130.58, 130.13, 128.96, 126.52, 126.30, 121.19, 121.13, 110.11, 109.91, 56.80, 56.40, 48.75, 36.55, 36.33, 35.80, 25.92, 22.54, 22.48, 19.53, 19.34.

### EXAMPLE 432

### Synthesis of N-(2-(N-Methylamino)-5-(2-tolyl)pyrimidin-4-yl)-L-4-(N,N-dimethylcarbarnyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =10.0-8.0 (br, 1H), 9.42 (bs, 1H), 8.24 (s, 1H), 7.4-6.8 (m, 10H), 5.93 (m, 1H), 4.85 (m, 1H), 3.2-2.8 (m, 1H), 3.37 (m, 1H), 3.12 (s, 1.5H), 3.11 (s, 1.5H), 3.03 (s, 1.5H), 3.02 (s, 1.5H), 2.95 (s, 3H), 2.13 (s, 1.5H), 1.83 (s, 1.5H).

### EXAMPLE 433

### Synthesis of N-(2-(2-tolyl)-5-(2-tolyl)pyrimidin-4-yl)-L-4-(N,N-dimethylcarbamyloxy)phenylalanine

Tyrosine tert-butyl ester (Bachem) was sequentially converted via Methods U, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with o-tolyl boronic acid (Aldrich) via Method KKK. The product of this coupling was converted via Method ZZ to give the title compound.

Physical data were as follows:

H NMR (CDCl₃): δ =8.14 (d, 1H), 7.68 (d, 1H), 7.4-6.8 (m, 12H), 5.42 (m, 1H), 4.94 (m, 1H), 3.11 (s, 3H), 3.02 (s, 3H), 3.4-2.8 (m, 2H), 2.49 (s, 3H), 2.11 (s, 1.5H), 1.91 (s, 1.5H).

### EXAMPLE 434

### Synthesis of N-(2-(N-Methyl-N-2-hydroxyethylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with 2-(methylamino)-ethanol (Aldrich) via Method CC to give a product that was coupled with o-tolyl boronic acid (Aldrich) via Method DD. The product of this coupling was converted via Method ZZ to give the title compound.

Physical data were as follows:

¹H NMR (CD₃OD): δ=7.4-6.94 (m, 4H), 4.82 (m, 1H), 3.8 (brs, 4H), 3.23/3.26 (s, rotamers, 3H), 2.98/3.7 (s, rotamers, 6H), 1.93/2.14 (s, rotamers, 3H).

### EXAMPLE 435

### Synthesis of N-(2-(N-Methyl-N-2-methyl propylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl isobutylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =10.5-9.8 (br, 1H), 7.63 (d, 1H), 7.3-6.8 (m, 8H), 6.35 (m, 1H), 4.65 (m, 1H), 3.6-2.8 (m, 4H), 3.08 (s, 3H), 3.01 (s, 6H), 2.13 (s, 1.5H), 2.06 (bs, 1H), 1.25 (s, 1.5H), 0.9 (s, 6H).

¹³C NMR (CDCl₃): δ =174.13, 173.97, 159.17, 158.9, 154.7, 153.99, 149.96, 142.00, 137.76, 137.53, 134.50, 134.33, 130.80, 130.58, 130.15, 128.95, 126.51, 126.30, 121.15, 121.11, 110.25, 109.99, 57.46, 56.90, 56.51, 36.89, 36.55, 36.32, 27.08, 19.87, 19.53, 19.38.

### EXAMPLE 436

### Synthesis of N-(2-(N-Methyl-N-propylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-propylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =10.5-9.5 (br, 1H), 7.6 (d, 1H), 7.38-6.7 (m, 8H), 6.3 (m, 1H), 4.7 (m, 1H), 3.7-3.0 (m, 4H), 3.09 (s, 3H), 3.01 (s, 6H), 2.13 (s, 1.5H), 1.92 (s, 1.5H), 1.59 (bs, 2H), 0.89 (bs, 3H).

¹³C NMR (CDCl₃): δ =174.22, 174.06, 159.26, 159.0, 154.7, 153.76, 149.97, 142.22, 137.78, 137.53, 134.53, 134.36, 130.80, 130.73, 130.51, 130.12, 128.93, 126.50, 126.30, 121.16, 121.10, 110.13, 109.87, 56.90, 56.52, 51.72, 36.55, 36.33, 35.96, 20.45, 19.56, 19.37, 11.06.

### EXAMPLE 437

### Synthesis of N-(2-(N-Dimethylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N,N-dimethylamine (Aldrich) via Method CC to give a product that was coupled with o-tolylboronic acid (Aldrich) via Method DD. The product of this coupling was converted via Method ZZ to give the title compound.

Physical data were as follows:

¹H NMR (CDCl₃): δ =11.0-9.5 (br, 1H), 7.62 (d, 1H), 7.3-6.8 (m, 8H), 6.22 (m, 1H), 4.72 (m, 1H), 3.5-3.0 (m, 2H), 3.8 (s, 6H), 3.01 (s, 3H), 2.12 (s, 1.5H), 1.94 (s, 1.5H).

¹³C NMR (CDCl₃): δ =174.49, 174.3, 159.4, 158.93, 154.72, 149.93, 140.30, 137.75, 137.60, 134.67, 134.50, 130.92, 130.80, 130.51, 130.11, 128.87, 126.48, 126.32, 121.15, 121.08, 109.87, 109.69, 56.86, 56.49, 37.51, 36.87, 36.55, 36.34, 19.50, 19.38.

### EXAMPLE 438

### Synthesis of N-(2-(N-Methyl-N-cyclohexylamino)-5-(3-pyridyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-cyclohexylamine (Aldrich) via Method CC to give a product that was coupled with 3-pyridyl boronic acid 1,3-propanediol cyclic ester (Lancaster Synthesis) via Method DD. The product of this coupling was converted via Method HH to give the title compound.

Physical data were as follows:

¹H NMR (CD₃OD): δ=8.83-8.78 (m, 1H), 8.56 (brs, 1H), 8.09-7.95 (m, 2H), 7.76-7.73 (m, 1H), 7.22 (d, 2H), 7.06 (d, 2H), 4.85 (m, 1H), 3.45-3.38 (m, 1H), 3.18-3.11 (m, 4H), 3.06 (s, 3H), 2.99 (sm, overlapping 4H), 1.92 (m, 2H), 1.76-1.57 (m, 8H).

¹³C NMR (CD₃OD): δ=173.7, 161.5, 161.4, 160.9, 157.0, 152.0, 146.0, 145.7, 145.6, 143.3, 136.0, 132.2, 131.3, 128.1, 123.4, 107.8, 57.8, 57.4, 36.8, 36.6, 36.1, 30.6, 30.0, 26.4, 26.2.

### EXAMPLE 439

### Synthesis of N-(5-(2-phenyl-2,2-difluoroethyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 1-Trifluoromethanesulfonyloxy-2,2-difluoro-2-phenylethane was sequentially converted via Methods LL, OO and QQ into 4,6-dichloro-5-(2,2-difluoro-2-phenylethyl)pyrimidine. L-4-(N,N-Dimethylcarbamyloxy)-phenylalanine tert-butyl ester and 4,6-dichloro-5-(2,2-difluoro-2-phenylethyl)pyrimidine were coupled via Method RR, and the product of this coupling was sequentially converted via Methods TT and ZZ to give the title compound.

Physical data were as follows:

¹H NMR (CD₃OD): δ=8.37 (s, 1H), 7.79 (s, 1H), 7.44 (s, 5H), 7.25 (d, 2H), 6.98 (d, 2H), 5.07 (dd, 1H), 3.62-3.32 (m, 3H), 3.14 (dd, 1H) 3.08 (s, 3H), 2.96 (s, 3H).

### EXAMPLE 440

### Synthesis of N-(5-(2-phenyl-2,2-difluoroethyl)-6-chloropyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 1-Trifluoromethanesulfonyloxy-2,2-difluoro-2-phenylethane was sequentially converted via Methods LL, OO and QQ into 4,6-dichloro-5-(2,2-difluoro-2-phenylethyl)pyrimidine. L-4-(N,N-Dimethylcarbamyloxy)-phenylalanine tert-butyl ester and 4,6-dichloro-5-(2,2-difluoro-2-phenylethyl)pyrimidine were coupled via Method RR, and the product of this coupling was converted via Method ZZ to give the title compound.

Physical data were as follows:

¹H NMR (CD₃OD): δ=8.18 (s, 1H), 7.42-7.41 (m, 5H), 7.26 (d, 2H), 7.0 (d, 2H), 5.03 (dd, 1H), 3.72-3.45 (m, 2H), 3.34 (dd, 1H), 3.19 (dd, 1H), 3.08 (s, 3H), 2.96 (s, 3H).

### EXAMPLE 441

### Synthesis of N-(5-(2-phenylethyl)pyrimidin-4-yl)-L-4-(N,N-dimethylcarbamyloxy)phenylalanine

4(3H)-Pyrimidinone (Aldrich) was sequentially converted via Methods N and O into 4-chloro-5-iodopyrimidine. L-Phenylalanine tert-butyl ester hydrochloride (Bachem) and 4-chloro-5-iodopyrimidine were coupled via Method P. The product of this reaction was converted via Method W to a product that was sequentially converted via Methods X and HH to give the title compound.

Physical data were as follows:

¹H NMR (CD₃OD): δ=8.55 (d, 1H), 7.64 (d, 1H), 7.35-7.19 (m, 8H), 7.01-6.98 (m, 2H), 5.46-5.41 (m, 1H), 5.34-3.60 (m, 1H), 3.29-3.23 (m, 1H), 2.94-2.75 (m, 4H).

¹³C NMR (CD₃OD): δ=174.3, 164.3, 151.5, 141.8, 141.7, 139.2, 131.0, 130.6, 130.5, 130.4, 128.9, 128.4, 120.6, 57.8, 38.4, 34.0, 30.7.

### EXAMPLE 442

### Synthesis of N-(2-(N-Methyl-N-cyclohexylamino)pyrimidin-4-yl)-L-4-(N,N-dimethylcarbamyloxy)phenylalanine

Tyrosine tert-butyl ester (Bachem) was sequentially converted via Methods U, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-cyclohexylamine (Aldrich) via Method CC to give a product which was sequentially converted via Methods MMM and ZZ to give the title compound.

Physical data were as follows:

¹H NMR (CDCl₃): δ =11.20 (bs, 2H), 8.44 (s, 1H), 7.76 (bs, 1H), 7.50 (br, 1H), 7.18 (d, 2H), 6.96 (d, 2H), 5.91 (bs, 1H), 4.83 (bs, 1H), 4.53 (br, 1H), 3.20 (m, 2H), 3.08 (s, 3H), 2.98 (s, 6H), 2.00-1.00 (m, 10H).

¹³C NMR (CDCl₃): δ =176.18, 171.50, 167.75, 162.44, 156.31, 154.49, 151.52, 135.83, 131.61, 122.85, 58.04, 56.87, 38.02, 37.79, 31.16, 31.00, 26.68.

### EXAMPLE 443

### Synthesis of N-(5-Propylpyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P, and the product of this coupling was sequentially converted via Methods II, JJ and KK to give the title compound.

Physical data were as follows:

¹H NMR (CD₃OD): δ=8.51 (s, 1H), 7.97 (s, 1H), 7.26 (d, 2H), 6.97 (d, 2H), 5.36 (m, 1H), 3.51 (m, 1H), 3.23 (m, 1H), 3.16 (s, 3H), 2.95 (s, 3H), 2.47 (m, 2H), 1.57 (m, 2H), 0.99 (m, 3H).

¹³C NMR (CD₃OD): δ=173.48, 163.61, 151.97, 150.75, 140.68, 135.74, 133.14, 131.30, 123.02, 120.85, 56.96, 36.99, 36.76, 36.58, 29.87, 21.02, 13.67.

### EXAMPLE 444

### Synthesis of N-(5-(2-Methoxyphenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with 2-methoxyphenyl boronic acid (Lancaster Synthesis) via Method Q. The product of this coupling was converted via Method HH to give the title compound.

Physical data were as follows:

¹H NMR (CD₃OD): δ=8.64 (s, 1H), 8.05 (s, 1H), 7.61-7.55 (m, 1H), 7.27-7.13 (m, 5H), 6.99 (d, 2H), 5.36-5.32 (m, 1H), 3.73 (s, 3H), 3.46-3.40 (m, 1H), 4.20-3.13 (m, 4H), 3.02 (s, 3H).

¹³C NMR (CD₃OD): δ=173.8, 163.5, 159.5, 157.5, 152.8, 152.1, 143.0, 135.9, 134.2, 133.9, 132.2, 123.8, 123.4, 120.5, 120.0, 113.7, 57.5, 57.1, 37.9, 37.7, 37.5.

### EXAMPLE 445

### Synthesis of N-(5-(2-Fluorophenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with 2-fluorophenyl boronic acid (Lancaster Synthesis) via Method Q. The product of this coupling was converted via Method HH to give the title compound.

### EXAMPLE 446

### Synthesis of N-(2-(N-Methyl-N-isopropylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-isopropylamine (Aldrich) via Method CC to give a product that was coupled with o-tolyl boronic acid (Aldrich) via Method DD.

The product of this coupling was converted via Method ZZ to give the title compound.

Physical data were as follows:

¹H NMR (CDCl₃): δ =10.5-9.5 (br, 1H), 7.59 (d, 1H), 7.30-6.70 (m, 8H), 6.3 (m, 1H), 4.92 (bs, 1H), 4.7 (m, 1H), 3.50-3.0 (m, 2H), 3.08 (s, 3H), 3.00 (s, 3H), 2.83 (s, 3H), 2.13 (s, 1.5H), 1.93 (s, 1.5H) 1.15 (d, 6H).

¹³C NMR (CDCl₃): δ =174.31, 174.15, 159.21, 158.95, 154.70, 153.41, 149.92, 141.98, 137.79, 137.56, 134.59, 134.41, 130.59, 130.17, 128.95, 126.51, 126.32, 121.15, 110.26, 110.02, 56.87, 56.50, 46.86, 36.82, 36.55, 36.31, 28.18, 19.50, 19.39.

### EXAMPLE 447

### Synthesis of N-(2-(N-Isopropylamino)-5-(2-tolyl)pyrimidin-4-yl)-L-4-(N,N-dimethylcarbamyloxy)phenylalanine

Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with isopropylamine (Aldrich) via Method CC to give a product that was coupled with o-tolyl boronic acid (Aldrich) via Method DD. The product of this coupling was converted via Method ZZ to give the title compound.

Physical data were as follows:

¹H NMR (CDCl₃): δ =9.57 (s, 1H), 8.31 (s, 1H), 7.40-6.80 (m, 8H), 6.19 (m, 1H), 4.79 (m, 1H), 4.15 (m, 1H), 3.4-3.0 (m, 2H), 3.10 (s, 3H), 3.01 (s, 3H), 2.16 (s, 1.5H), 1.41 (s, 1.5H), 1.24 (s, 6H).

¹³C NMR (CDCl₃): δ =176.07, 175.8, 166.23, 160.23, 159.99, 154.79, 153.50, 158.06, 139.38, 137.86, 137.66, 134.10, 133.93, 130.77, 130.61, 130.26, 130.01, 129.25, 126.71, 126.50, 121.46, 121.36, 109.59, 109.37, 56.77, 56.22, 43.31, 36.57, 36.34, 22.12, 21.96, 19.47, 19.22.

### EXAMPLE 448

### Synthesis of N-(5-(2-phenylethyl)pyrimidin-4-yl)-L-4-(N,N-dimethylcarbamyloxy)phenylalanine Isopropyl Ester

L-Tyrosine was sequentially converted via Methods LLL, UU, VV and WW into L-4-(N,N-dimethylcarbamyloxy)phenylalanine isopropyl ester. 4(3H)-Pyrimidinone (Aldrich) was sequentially converted via Methods N and O to 4-chloro-5-iodopyrimidine. L-4-(N,N-Dimethylcarbamyloxy)phenylalanine isopropyl ester and 4-chloro-5-iodopyrimidine were coupled via Method P, and the coupled product was converted via Method OOO to a product, which was converted via Method X to give the title compound.

Physical data were as follows:

1H NMR (CDCl₃): δ =8.50 (s, 1H), 7.91 (s, 1H), 7.31-7.20 (m, 3H), 7.42-7.00 (m, 6H), 5.19-5.17 (m, 1H), 5.08-5.02 (m, 2H), 3.23-3.17 (m, 2H), 3.06 (s, 3H), 2.99 (s, 3H), 2.83-2.78 (m, 2H), 2.65-2.60 (m, 2H), 1.75-1.23 (m, 6H).

¹³C NMR (CDCl₃): δ =171.8, 159.2, 156.7, 153.5, 150.7, 140.5, 130.3, 128.7, 128.5, 126.4, 121.8, 117.1, 69.4, 54.2, 36.9, 36.6, 36.5, 33.6, 29.8, 21.7, 21.6.

### EXAMPLE 449

### Synthesis of N-(3-(N-Methyl-N-4-toluenesulfonylamino)pyrazin-2-yl)-L-phenylalanine Isopropyl Ester

L-Phenylalanine (Aldrich) was converted via Method LLL to L-phenylalanine isopropyl ester hydrochloride. 2,3-Dichloropyrazine (Lancaster) was converted via Method QQQ and RRR into 2-chloro-3-nitropyrazine. L-Phenylalanine isopropyl ester hydrochloride and 2-chloro-3-nitropyrazine were coupled via Method BBB, and the product of this coupling was sequentially converted via Methods CCC, DDD and EEE to give the title compound.

Physical data were as follows:

¹H NMR (CDCl₃): δ =7.91 (d, 1H), 7.59 (d, 2H), 7.51 (d, 1H), 7.31-7.23 (m, 7H), 6.08 (d, 1H), 5.01-4.97 (m, 1H), 4.92-4.89 (m, 1H) 3.24 (d, 2H), 2.97 (s, 3H), 2.43 (s, 3H), 1.21-1.12 (m, 6H).

¹³C NMR (CDCl₃): δ =167.32, 147.440, 139.85, 137.38, 133.25, 131.98, 128.68, 126.17, 125.17, 125.06, 124.41, 124.11, 122.58, 64.38, 50.65, 33.49, 32.41, 17.16, 17.08, 17.03.

### EXAMPLE 450

### Synthesis of N-(5-(2-phenylethyl)pyrimidin-4-yl)-L-phenylalanine Isopropyl Ester

L-Phenylalanine isopropyl ester hydrochloride was prepared by Method LLL. 4(3H)-Pyrimidinone (Aldrich) was sequentially converted via Methods N and O to 4-chloro-5-iodopyrimidine. L-Phenylalanine isopropyl ester hydrochloride and 4-chloro-5-iodopyrimidine were coupled via Method P and the coupled product sequentially converted via Methods OOO and X to give the title compound.

Physical data were as follows:

¹H NMR (CDCl₃): δ =8.51 (s, 1H), 7.92 (s, 1H), 7.30-7.15 (m, 5H), 7.14-7.06 (m, 4H), 5.16 (m, 1H), 5.09-5.01 (m, 2H), 3.31-3.16 (m, 2H), 2.79-2.74 (m, 2H), 2.62-2.57 (m, 2H), 1.15-1.20 (m, 6H).

¹³C NMR (CDCl₃): δ =171.7, 159.1, 156.7, 153.5, 140.5, 136.1, 129.4, 128.6, 128.5, 128.3, 127.1, 126.4, 117.0, 69.3, 54.2, 37.6, 33.7, 30.0, 21.7, 21.6.

### EXAMPLE 451

### Synthesis of N-(5-(N-Methyl-N-3-pyridinesulfonylamino)pyrimidin-4-yl)-L-4-(4-methylpiperazin-1-ylcarbonyloxy)phenylalanine

L-Tyrosine tert-butyl ester (Bachem) was sequentially converted via Methods UU, XX and YY into L-4-(4-methylpiperazin-1-ylcarbonyloxy)-phenylalanine tert-butyl ester. 5-Nitrouracil (Aldrich) was converted via Method AAA into 2,4-dichloro-5-nitropyrimidine. L-4-(4-Methylpiperazin-1-ylcarbonyloxy)phenylalanine tert-butyl ester and 2,4-dichloro-5-nitropyrimidine were coupled via Method BBB, and the product of this coupling was sequentially converted via Methods CCC, DDD (using 3-chlorosulfonylpyridine), EEE and ZZ to give the title compound.

### EXAMPLE 452

### Synthesis of N-(2-(N-Methyl-N-cyclohexylamino)-5-(2-tolyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. L-4-(N,N-Dimethylcarbamyloxy)-phenylalanine tert-butyl ester and 5-bromo-2,4-dichloropyrimidine (Aldrich) were coupled via Method BB. The product of this reaction was reacted with N-methyl-N-cyclohexylamine (Aldrich) via Method CC to give a product that was coupled with o-tolyl boronic acid (Aldrich) via Method DD.

The product of this coupling was converted via Method ZZ to give the title compound.

Physical data were as follows:

1H NMR (CDCl₃): δ =10.0-9.08 (br, 1H), 7.55 (s, 0.5H), 7.52 (s, 0.5H), 7.20-6.31 (m, 8H), 6.36 (br, 1H), 4.69 (m, 2H), 3.40 (m, 1H), 3.15 (m, 1H), 3.06 (brs, 3H), 2.98 (brs, 3H), 2.84 (brs, 3H), 2.11 (s, 1.5H), 2.00-1.00 (brm, 11.5H).

¹³C NMR (CDCl₃): δ =164.10, 159.20, 159.00, 154.79, 153.50, 150.03, 137.68, 137.48, 134.48, 130.66, 130.22, 129.01, 126.62, 126.40, 121.16, 110.20, 57.00, 56.58, 55.50, 36.62, 36.39, 29.91, 29.52, 25.41, 19.60, 19.65.

### EXAMPLE 453

### Synthesis of N-(5-(2-Tolyl)pyrimidin-4-yl)-L-4-(N,N-dimethylcarbamyloxy)phenylalanine Isopropyl Ester

L-Tyrosine (Aldrich) was sequentially converted via Methods LLL, U, VV and WW into L-4-(N,N-dimethylcarbamyloxy)phenylalanine isopropyl ester. 4(3H)-Pyrimidinone (Aldrich) was sequentially converted via Methods N and O into 4-chloro-5-iodopyrimidine. L-4-(N,N-dimethylcarbamyloxy)-phenylalanine isopropyl ester and 4-chloro-5-iodopyrimidine were coupled via Method P. The product of this coupling was reacted with o-tolyl boronic acid via Method Q to afford the title compound.

Physical data were as follows:

¹H NMR (CDCl₃): δ =8.58 (s, 1H), 7.99 (s, 1H), 7.76-7.33 (m, 3H), 7.13 (m, 0.5H), 7.03-6.95 (m, 4H), 4.97-4.87 (m, 3H), 3.08-2.99 (m, 8H), 2.09 (s, 2H), 1.92 (s, 1.5H), 1.24-1.12 (m, 6H).

¹³C NMR (CDCl₃): δ =171.4, 171.2, 158.8, 158.5, 157.5, 154.7, 153.6, 153.5, 150.5, 137.1, 137.0, 132.9, 132.3, 132.5, 130.8, 130.7, 130.0, 129.8, 129.7, 128.9, 126.6, 126.5, 121.6, 119.5, 119.4, 69.0, 54.5, 54.0, 36.9, 36.8, 36.6, 36.4, 21.65 21.60, 19.3, 19.2.

### EXAMPLE 454

### Synthesis of N-(5-(3-Nitrophenyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P, and the coupled product was reacted with 3-nitrophenyl boronic acid (Aldrich) via Method T. The product of this coupling was converted via Method HH to give the title compound.

Physical data were as follows:

¹H NMR (CD₃OD): δ=8.67 (s, 1H), 8.41-8.38 (m, 1H), 8.28-8.27 (m, 1H), 8.17 (s, 1H), 7.82-7.77 (m, 1H), 7.67-7.65 (m, 1H), 7.20 (d, 2H), 7.02 (d, 2H), 5.33-5.28 (m, 1H), 3.47-3.411 (m, 1H), 3.12-3.04 (m, 4H), 2.97 (s, 3H).

¹³C NMR (CD₃OD): δ=173.7, 163.6, 157.6, 152.8, 152.7, 151.2, 143.8, 137.4, 136.3, 134.2, 133.1, 132.2, 126.7, 126.3, 124.0, 120.3, 58.0, 37.7, 37.6, 37.5.

### EXAMPLE 455

### Synthesis of N-(5-(3-Pyridyl)pyrimidin-4-yl)-L-4-(N,N-dimethylcarbamyloxy)phenylalanine

L-Tyrosine tert-butyl ester (Bachem) was sequentially converted via Methods U, VV and WW into L-4-(N,N-dimethylcarbamyloxy)-phenylalanine tert-butyl ester. 4(3H)-Pyrimidinone (Aldrich) was sequentially converted via Methods N and O to 4-chloro-5-iodopyrimidine. L-4-(N,N-Dimethylcarbamyloxy)phenylalanine tert-butyl ester and 4-chloro-5-iodopyrimidine were coupled via Method P, and the coupled product was reacted with 3-pyridyl boronic acid 1,3-propanediol cyclic ester (Lancaster Synthesis) via Method Q. The product of this coupling was converted via Method HH to give the title compound.

### EXAMPLE 456

### Synthesis of N-(5-(2-phenylethyl)pyrimidin-4-yl)-L-4-(N,N-dimethylcarbamyloxy)phenylalanine

L-Tyrosine tert-butyl ester (Bachem) was sequentially converted via Methods U, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P. The product of this reaction was sequentially converted via Methods W, X, and HH to give the title compound.

Physical data were as follows:

¹H NMR (CD₃OD): δ=8.52 (s, 1H), 7.67 (s, 1H), 7.34-7.19 (m, 5H), 7.08-6.99 (m, 4H), 5.50-5.42 (m, 1H), 5.59-5.53 (m, 1H), 3.26-3.21 (m, 1H), 3.09 (s, 2H), 2.99 (s, 3H), 2.94-2.85 (m, 4H).

¹³C NMR (CD₃OD): δ=174.2, 164.2, 157.5, 152.7, 151.4, 141.8, 141.7, 136.5, 132.0, 130.5, 130.4, 128.4, 123.8, 120.5, 57.8, 37.9, 37.6, 37.5, 34.1, 30.6.

### EXAMPLE 457

### Synthesis of N-(2-N,N-Dimethylamino-5-(N-methyl-N-4-toluenesulfonylamino)pyrimidin-4-yl)-L-phenylalanine

5-Nitrouracil (Aldrich) was converted via Method AAA into 2,4-dichloro-5-nitropyrimidine. L-Phenylalanine tert-butyl ester (Bachem) and 2,4-dichloro-5-nitropyrimidine were coupled via Method BBB, and the product of this coupling was sequentially converted via Methods SSS (using dimethylamine), CCC, DDD, EEE and ZZ to give the title compound.

Physical data were as follows:

¹H NMR (CD₃OD): δ=8.15 (s, formate), 7.65 (m, 2H), 7.41 (d, 2H), 7.40-7.19 (m, 5H), 7.02-6.92 (m, 1H), 4.90 (m, 1H), 3.40-3.10 (m, 2H), 3.09-2.92 (m, 9H), 2.43 (s, 3H).

¹³C NMR (CD₃OD): δ=177.07, 159.64, 154.70, 152.25, 144.10, 141.97, 141.33, 140.25, 132.57, 129.02, 125.21, 124.82, 123.57, 123.42, 121.88, 107.64, 51.08, 33.71, 32.72, 31.76, 15.49.

### EXAMPLE 458

### Synthesis of N-(5-(2-Tolyl)pyrimidin-4-yl)-L-4-(N,N-dimethylcarbamyloxy)phenylalanine

L-Tyrosine tert-butyl ester (Bachem) was sequentially converted via Methods UU, VV and WW into L-4-(N,N-dimethylcarbamyloxy)-phenylalanine tert-butyl ester. 4(3H)-Pyrimidinone (Aldrich) was sequentially converted via Methods N and O into 4-chloro-5-iodopyrimidine. L-4-(N,N-Dimethylcarbamyloxy)phenylalanine tert-butyl ester and 4-chloro-5-iodopyrimidine were coupled via Method P and the coupled product was reacted with o-tolyl boronic acid (Aldrich) via Method Q. The product of this coupling was converted via Method HH to give the title compound.

Physical data were as follows:

¹H NMR (CD₃OD): δ=8.75-8.65 (d, 1H), 8.05-8.03 (d, 1H), 7.51-7.35 (m, 3H), 7.26-7.11 (m, 3H), 7.02-6.97 (m, 2H), 5.38-5.27 (m, 2H), 3.50-3.39 (m, 1H), 3.21-3.07 (m, 4H), 3.02 (s, 3H), 2.21-1.93 (s, 3H).

¹³C NMR (CD₃OD): δ=173.8, 173.6, 164.0, 163.8, 157.5, 152.7, 152.6, 143.0, 142.8, 139.7, 139.5, 136.1, 135.9, 133.2, 133.0, 132.4, 132.2, 132.1, 131.9, 131.1, 129.0, 128.9, 123.8, 123.7, 122.2, 122.0, 57.6, 57.4, 37.8, 37.7, 37.5, 37.4, 20.3, 2.2.

Additionally, using the procedures described herein and the appropriate starting materials, the following additional compounds can be prepared:
N-(2-(N-methyl-N-cyclohexylamino)-5-(2-methoxyphenyl)pyrimidin-4-yl)-L-4-(2,6-dimethoxyphenyl)phenylalanine (Example 459),
N-(2-(N-methyl-N-isopropylamino)-5-(2-fluorophenyl)pyrimidin-4-yl)-L-4-(2,6-dimethoxyphenyl)phenylalanine (Example 460),
N-(2-(N-methyl-N-isopropylamino)-5-(2-fluorophenyl)pyrimidin-4-yl)-L-4-(2-methoxyphenyl)phenylalanine (Example 461),
N-(2-(N-methyl-N-cyclohexylamino)-5-(2,6-difluorophenyl)pyrimidin-4-yl)-L-4-(2,6-difluorophenyl)phenylalanine (Example 462),
N-(2-(N-methyl-N-cyclohexylamino)-5-(2-hydroxymethylphenyl)pyrimidin-4-yl)-L-4-(2,6-dimethoxyphenyl)phenylalanine (Example 463),
N-(2-(N,N-bis-(2-hydroxyethyl)amino)-5-(2,4,6-trimethylphenyl)pyrimidin-4-yl)-L-4-(2,6-dimethoxyphenyl)phenylalanine (Example 464),
N-(2-(N-methyl-N-cyclohexylamino)-5-(2-trifluoromethylphenyl)pyrimidin-4-yl)-L-4-(2-cyanophenyl)phenylalanine (Example 465),
N-(2-(N-methyl-N-cyclohexylamino)-5-(3-thienyl)pyrimidin-4-yl)-L-4-(2,6-dimethoxyphenyl)phenylalanine (Example 466),
N-(2-(N-methyl-N-cyclohexylamino)-5-(2-thienyl)pyrimidin-4-yl)-L-4-(4-trifluoromethylphenyl)phenylalanine (Example 467),
N-(2-(N-methyl-N-cyclohexylamino)-5-(3-pyridyl)pyrimidin-4-yl)-L-4-(2,6-dimethoxyphenyl)phenylalanine (Example 468),
N-(2-(N-methyl-N-cyclohexylamino)-5-(3-nitrophenyl)pyrimidin-4-yl)-L-4-(2,6-dimethoxyphenyl)phenylalanine (Example 469),
N-(2-(N-methyl-N-cyclohexylamino)-5-(2,6-dichlorophenyl)pyrimidin-4-yl)-L-4-(2,6-dimethoxyphenyl)phenylalanine (Example 470),
N-(2-(N-methyl-N-cyclohexylamino)-5-(4-pyridyl)pyrimidin-4-yl)-L-4-(3-hydroxymethylphenyl)phenylalanine (Example 471),
N-(2-(N-ethyl-N-isopropylamino)-5-(2,6-dimethoxyphenyl)pyrimidin-4-yl)-L-4-(2,6-dimethoxyphenyl)phenylalanine (Example 472),
N-(2-(N-methyl-N-cyclohexylamino)-5-(2,3-dichlorophenyl)pyrimidin-4-yl)-L-4-(2,6-dimethoxyphenyl)phenylalanine (Example 473),
N-(2-(N-methyl-N-ethylamino)-5-(2,4,6-trimethylphenyl)pyrimidin-4-yl)-L-4-(2-cyanophenyl)phenylalanine (Example 474),
N-(2-(N-methyl-N-isopropylamino)-5-(2,4,6-trimethylphenyl)pyrimidin-4-yl)-L-4-(3-pyridyl)phenylalanine (Example 475),
N-(2-(N,N-bis-(2-hydroxyethyl)amino)-5-(2,4,6-trimethylphenyl)pyrimidin-4-yl)-L-4-(2-cyanophenyl)phenylalanine (Example 476),
N-(2-(N-methyl-N-(1-methylpiperidin-4-yl)amino)-5-(2-cyanophenyl)pyrimidin-4-yl)-L-4-(2,6-difluorophenyl)phenylalanine (Example 477),
N-(2-(N-ethyl-N-isopropylamino)-5-(2,4,6-trimethylphenyl)pyrimidin-4-yl)-L-4-(o-tolyl)phenylalanine (Example 478),
N-(2-(N-methyl-N-4-chlorophenylamino)-5-(2,4,6-trimethylphenyl)pyrimidin-4-yl)-L-4-(2,6-dimethoxyphenyl)phenylalanine (Example 479),
N-(5-(N-methyl-N-2-(phenyl)ethylamino)pyrimidin-4-yl)-L-4-(N,N-dimethylcarbamyloxy)phenylalanine (Example 480),
N-(5-(N-methyl-N-hexylamino)pyrimidin-4-yl)-L-4-(N,N-dimethylcarbamyloxy)phenylalanine (Example 481),
N-(5-(N-methyl-N-isopropylamino)pyrimidin-4-yl)-L-4-(N,N-dimethylcarbamyloxy)phenylalanine (Example 482),
N-(5-(N-methyl-N-isopropylamino)pyrimidin-4-yl)-L-4-(N,N-dimethylcarbamyloxy)phenylalanine (Example 483),
N-(5-(N-methyl-N-tert-butylamino)pyrimidin-4-yl)-L-4-(N,N-dimethylcarbamyloxy)phenylalanine (Example 484),
N-(5-(N-ethyl-N-isopropylamino)pyrimidin-4-yl)-L-4-(N,N-dimethylcarbamyloxy)phenylalanine (Example 485),
N-(5-(N-methyl-N-2-(4-pyridyl)ethyl-pyrimidin-4-yl)-L-4-(N,N-dimethylcarbamyloxy)phenylalanine (Example 486),
N-(5-(N-methyl-N-2-(phenyl)ethylamino)pyrimidin-4-yl)-L-4-(4-(2,6-dimethoxyphenyl)phenylalanine (Example 487),
N-(5-(N-methyl-N-hexylamino)pyrimidin-4-yl)-L-4-(2,6-dimethoxyphenyl)phenylalanine (Example 488),
N-(5-(N-methyl-N-isopropylamino)pyrimidin-4-yl)-L-4-(2,6-dimethoxyphenyl)phenylalanine (Example 489),
N-(5-(N-methyl-N-isopropylamino)pyrimidin-4-yl)-L-4-(2,6-dimethoxyphenyl)phenylalanine (Example 490),
N-(5-(N-methyl-N-tert-butylamino)pyrimidin-4-yl)-L-4-(2,6-dimethoxyphenyl)phenylalanine (Example 491),
N-(5-(N-ethyl-N-isopropylamino)pyrimidin-4-yl)-L-4-(2,6-dimethoxyphenyl)phenylalanine (Example 492),
N-(5-(N-methyl-N-2-(4-pyridyl)ethyl-pyrimidin-4-yl)-L-4-(2,6-dimethoxyphenyl)phenylalanine (Example 493),
N-(2-(N-methyl-N-cyclohexylamino)-5-ethylpyrimidin-4-yl)-L-4-(N,N-dimethylcarbamyloxy)phenylalanine (Example 494).

### EXAMPLE 495

### Synthesis of N-(4-(N,N-Di-n-hexylamino)-1,1-dioxo-1,2,5-thiadiazol-3-yl)-L-tyrosine

### Step A: Preparation of 3,4-Diethyloxy-1-oxo-1,2,5-thiadiazole and 3,4-Diethyloxy-1,1-dioxo-1,2,5-thiadiazole

The title intermediates were prepared according to the procedures described in R. Y. Wen et al, J. Org Chem. (1975) 40, 2743; and R. Y. Wen et al, Org Prep Proceed. (1969) 1, 255.

### Step B: Preparation of 4-(N,N-Di-n-hexylamino)-3-ethoxy-1,1-dioxo-1,2,5-thiadiazole

Dihexylamine (90 mg, 0.48 mmol) was added to a solution of 3,4-diethyloxy-1,1-dioxo-1,2,5-thiadiazole (100 mg, 0.48 mmol) in ethanol (5 mL) and the reaction stirred overnight at room temperature. The solvent was removed under reduced pressure and the residue absorbed onto silica gel, and purified by flash column chromatography (silica, hexane:EtOAc 3:1) to yield the title intermediate (120 mg, 72%).

Physical data were as follows:

MS (EI, m/e) 345.

### Step C: Preparation of N-(4-(N,N-Di-n-hexylamino)-1-dioxo-1,2,5-thiadiazol-3-yl)-L-tyrosine tert-Butyl Ester

A solution of 4-(N,N-di-n-hexylamino)-3-ethoxy-1,1-dioxo-1,2,5-thiadiazole (400 mg, 1.02 mmol) and L-tyrosine t-butyl ester (261 mg, 1.1 mmol) in EtOH (10 mL) was stirred at room temperature for 36 hrs. The solvent was removed under reduced pressure residue purified by flash column chromatography (silica, hexane:EtOAc 3:1 then 1:1) to give the title compound as a white waxy solid (400 mg, 73%).

Physical data were as follows:

Anal. Calcd. for C₂₇H₄₄N₄O₅S.0.55EtOAc: C, 59.93; H, 8.34; N, 9.57. Found: C, 59.84; H, 8.44; N, 9.62.

### Step D: Preparation of N-(4-(N,N-Di-n-hexylamino)-1,1-dioxo-1,2,5-thiadiazol-3-yl)-L-tyrosine

The compound from Step C (100 mg, 0.19 mmol) was dissolved in formic acid and the mixture stirred at room temperature for 36 hrs. Excess formic acid was removed under reduced pressure to yield the title compound as a white solid (90 mg, 98%).

Physical data were as follows:

Anal. Calcd. for C₂₃H₃₆N₄O₅S: C, 57.48; H, 7.55; N, 11.66. Found: C, 57.04; H, 7.23; N, 11.38.

### EXAMPLE 496

### Synthesis of N-(4-(N,N-Di-n-hexylamino)-1,1-dioxo-1,2,5-thiadiazol-3-yl)-L-4-(N,N-dimethylcarbamyloxy)phenylalanine

### Step A: Preparation of N-(4-(N,N-Di-n-hexylamino)-1,1-dioxo-1,2,5-thiadiazol-3-yl)-L-4-(N,N-dimethylcarbamyloxy)-phenylalanine tert-Butyl Ester

N-(4-(N,N-Di-n-hexylamino)-1,1-dioxo-1,2,5-thiadiazol-3-yl)-L-tyrosine tert-butyl ester (180 mg, 0.34 mmol.) was dissolved in pyridine (5 ml). Dimethylcarbamoyl chloride (108 mg, 1 mmol) was added dropwise and the mixture stirred at room temperature overnight. Pyridine was removed under high vacuum (low water bath temperature), the residue absorbed onto silica gel and purified by flash column chromatography (silica, hexane:EtOAc 2:1) to yield the title compound (140 mg, 68%).

### Step B: Preparation of N-(4-(N,N-Di-n-hexylamino)-1,1-dioxo-1,2,5-thiadiazol-3-yl)-L-4-(N,N-dimethylcarbamyloxy)-phenylalanine

The compound from Step A (140 mg, 0.23 mmol) was dissolved in formic acid and the mixture stirred at room temperature overnight. Excess formic acid was removed under reduced pressure to yield the title compound as a white solid (110 mg, 87%).

Physical data were as follows:

Anal. Calcd. for C₂₆H₄₁N₅O₆S: C, 56.6; H, 7.49; N, 12.69. Found: C, 56.67; H, 7.4; N, 12.46.

### EXAMPLE 497

### Synthesis of N-(4-(N,N-Di-n-hexylamino)-1,1-dioxo-1,2,5-thiadiazol-3-yl)-L-4-(4-methylpiperazin-1-ylcarbonyloxy)phenylalanine

### Step A: Preparation of N-(4-(N,N-Di-n-hexylamino)-1,1-dioxo-1,2,5-thiadiazol-3-yl)-L-4-(4-methylpiperazin-1-ylcarbonyloxy)phenylalanine tert-Butyl Ester

A solution of N-(4-(N,N-di-n-hexylamino)-1,1-dioxo-1,2,5-thiadiazol-3-yl)-L-tyrosine tert-butyl ester (500 mg, 0.93 mmol), and p-nitrophenyl chloroformate (179 mg, 0.89 mmol) in dichloromethane (20 mL) was cooled to 0° C. under an argon atmosphere. Triethylamine (235 mg, 2.32 mmol) was added dropwise and the mixture stirred at 0° C. for 30 mins, then allowed to warm to room temperature for a further 40 mins. The mixture was recooled to 0° C. and N-methylpiperazine (90 mg, 0.89 mmol) added. The mixture was allowed to warm to room temperature and stirred for three hours. The mixture was diluted with diethyl ether (150 mL) and the organic solution washed with 10% potassium carbonate solution until no further yellow color was produced in the aqueous phase. The organic layer was separated, dried (MgSO₄) and the solvent removed under reduced pressure. The residue was purified by flash column chromatography (silica, EtOAc:MeOH:Et₃N 94:5:1) to give the title compound as a pale yellow foam (310 mg, 50%).

Physical data were as follows:

Anal. Calcd. for C₃₃H₅₄N₆O₆S: C, 59.79; H, 8.21; N, 12.68. Found: C, 59.47; H, 8.25; N, 12.49

### Step B: Preparation of N-(4-(N,N-Di-n-hexylamino)-1,1-dioxo-1,2,5-thiadiazol-3-yl)-L-4-(4-methylpiperazin-1-ylcarbonyloxy)phenylalanine

The compound from Step A (200 mg, 0.3 mmol) was dissolved in formic acid (5 mL) and the mixture stirred at room temperature for 48 hrs. Excess formic acid was removed under reduced pressure and the residue recrystallized from EtOAc/MeOH to yield the title compound as an off-white solid (120 mg, 67%).

Physical data were as follows:

Anal. Calcd. for C₂₉H₄₆N₆O₆S.0.75H₂O: C, 56.15; H, 7.72; N, 13.55. Found: C, 56.1; H, 7.44; N, 13.46.

### EXAMPLE 498

### Synthesis of N-[4-(2-(3-Methylphenylaminocarbonylamino)eth-1-ylamino)-1,1-dioxo-1,2,5-thiadiazol-3-yl]-L-4-(N,N-dimethylcarbamyloxy)phenylalanine

### Step A: Preparation of N-(4-Ethoxy-1,1-dioxo-1,2,5-thiadiazol-3-yl) L-tyrosine tert-Butyl Ester

A solution of 3,4-diethyloxy-1,1-dioxo-1,2,5-thiadiazole (400 mg, 1.94 mmol) and L-tyrosine t-butyl ester (1.25 g, 5.2 mmol) in ethanol (25 mL) was stirred at room temperature overnight. Solvent was removed under reduced pressure and the product used in further transformations without further purification (Yield 790 mg).

### Step B: Preparation of 2-(3-Methylphenylaminocarbonylamino)eth-1-ylamine

N-Boc-Ethylene diamine (800 mg, 5 mmol) and m-tolyl isocyanate (665 mg, 5 mmol) were dissolved in acetonitrile and the mixture stirred at room temperature for 4 hrs. Solvent was removed under reduced pressure and the residue absorbed onto silica gel; prior to purification by flash column chromatography (silica, hexane:EtOAc 1:1) to yield the desired compound as a white solid (300 mg, 21%) (MS (+ESI, m/e) 294 (M+H)⁺). The N-Boc protected compound (300 mg, 1.02 mmol) was dissolved in formic acid (10 ml) and the mixture stirred at room temperature overnight. Excess acid was removed to yield the formate salt of the title compound as a white foam (210 mg).

### Step C: Preparation of N-[4-(2-(3-Methylphenylamino-carbonylamino)eth-1-ylamino)-1,1-dioxo-1,2,5-thiadiazol-3-yl]-L-tyrosine tert-Butyl Ester

To a solution of N-(4-ethoxy-1,1-dioxo-1,2,5-thiadiazol-3-yl)-L-tyrosine tert-butyl ester from Step A (150 mg, 0.38 mmol) and the formate salt of 2-(3-methylphenylaminocarbonylamino)eth-1-ylamine from Step B (210 mg, 0.89 mmol) in ethanol (10 mL) was added triethylamine (133 mg, 1.44 mmol). The reaction was stirred at room temperature overnight. Solvent was removed under reduced pressure and the residue purified by flash column chromatography (silica, 5% MeOH in EtOAc) to give the title compound (130 mg, 91%).

Physical data were as follows:

MS (+ESI, m/e) 545 (M+H)⁺.

### Step D: Preparation of N-[4-(2-(3-Methylphenylamino-carbonylamino)eth-1-ylamino)-1,1-dioxo-1,2,5-thiadiazol-3-yl]-L-4-(N,N-dimethylcarbamyloxy)phenylalanine tert-Butyl Ester

The intermediate from Step C (130 mg, 0.24 mmol) was dissolved in pyridine (5 mL). Dimethylcarbamoyl chloride (77 mg, 0.72 mmol) was added dropwise and the mixture heated at 50° C. under an argon atmosphere overnight. Pyridine was removed under reduced pressure, the residue absorbed onto silica gel and purified by flash column chromatography (silica, hexane:EtOAc 1:2, then 5% MeOH in EtOAc) to yield the title compound (140 mg, 93%).

Physical data were as follows:

MS (+ESI, m/e) 616 (M+H)⁺.

### Step E: Preparation of N-[4-(2-(3-Methylphenylamino-carbonylamino)eth-1-ylamino)-1,1-dioxo-1,2,5-thiadiazol-3-yl]-L-4-(N,N-dimethylcarbamyloxy)phenylalanine

The compound from Step D (120 mg, 0.19 mmol) was dissolved in formic acid (10 mL) and the mixture stirred at room temperature for 36 hrs. Excess acid was removed to yield the title compound as a pale yellow foam (100 mg, 93%).

Physical data were as follows:

MS (+ESI, m/e) 560 (M+H)⁺.

### EXAMPLE 499

### Synthesis of N-(4-(N,N-Dimethylamino)-1-oxo-1,2,5-thiadiazol-3-yl)-L-4-(N,N-dimethylcarbamyloxy)phenylalanine tert-Butyl Ester

### Step A: Preparation of N-(4-Ethoxy-1-oxo-1,2,5-thiadiazol-3-yl)-L-tyrosine tert-Butyl Ester

A solution of 3,4-diethoxy-1-oxo-1,2,5-thiadiazole (1 g, 0.52 mmol) and L-tyrosine t-butyl ester (1.25 g, 0.52 mmol) in ethanol (25 mL) was stirred at room temperature for 60 hr. Solvent was removed under reduced pressure and the residue purified by flash column chromatography (silica, hexane:EtOAc 1:1 to give the title intermediate (1.75 g, 88%).

Physical data were as follows:

MS (+ESI, m/e) 382 (M+H)⁺.

### Step B: Preparation of N-(4-Ethoxy-1-oxo-1,2,5-thiadiazol-3-yl)-L-4-(N,N-dimethylcarbamyloxy)phenylalanine tert-Butyl Ester

The intermediate from Step A (400 mg, 1.05 mmol) was dissolved in pyridine (10 mL) and dimethylcarbamoyl chloride (338 mg, 3.15 mmol) was added. The reaction was stirred at room temperature under an inert atmosphere overnight. TLC indicated large amounts of unreacted starting material so the mixture was heated at 50° C. for a further 48 hrs. Excess pyridine was removed under reduced pressure and the residue purified by flash column chromatography (silica, hexane:EtOAc 1:1 to give the title intermediate (280 mg, 59%).

Physical data were as follows:

MS (+ESI, m/e) 453 (M+H).

### Step C: Preparation of N-(4-(N,N-Dimethylamino)-1-oxo-1,2,5-thiadiazol-3-yl)-L-4-(N,N-dimethylcarbamyloxy)phenylalanine tert-Butyl Ester

A 2M solution of dimethylamine in THF (5 mL, 10 mmol) was added to a solution of the compound from Step B (180 mg, 0.35 mmol) in ethanol (10 mL). The reaction was stirred at room temperature overnight and solvent removed under reduced pressure. Residue was purified by flash column chromatography (silica, EtOAc:MeOH:Et₃N 90:10:1) to give the title compound as a white foam (140 mg, 88%).

Physical data were as follows:

Anal. Calcd. for C₂₂₀H₂₉N₅O₅S: C, 53.2; H, 6.47; N, 15.51. Found: C, 52.94; H, 6.18; N, 15.34.

### EXAMPLE 500

### Synthesis of N-(5-(2,2,2-Trifluoroethyl)pyrimidin-4-yl)-L-4-(2,6-dimethoxyphenyl)phenylalanine

Substituting L-4-(2,6-dimethoxyphenyl)phenylalanine methyl ester from Method TTT for L-4-(N,N-dimethylcarbamyloxy)phenylalanine tert-butyl ester and following the procedure described for the preparation of Example 408 yielded the title compound.

Physical data were as follows:

¹H NMR (CD₃OD): δ 8.41 (s, 1H), 8.05 (s, 1H), 7.24 (t, 1H), 7.2 (d, 2H), 7.1 (d, 2H), 6.67 (d, 2H), 5.1 (dd, 1H), 3.65 (s, 6H), 3.61-3.42 (m, 2H), 3.36 (dd, 1H), 3.2 (dd, 1H).

¹³C NMR (CD₃OD): δ 175.8, 162.3, 159.2, 157.9, 155.8, 136.9, 134.4, 132.2, 130.0, 129.5, 127.4, 120.9, 109.6, 105.7, 56.8, 56.2, 37.9, 32.6.

### EXAMPLE 501

### Synthesis of N-(2-(N-Cyclohexyl-N-methyl)-5-(2-tolyl)pyrimidin-4-yl)-L-4-(2,6-dimethoxyphenyl)phenylalanine

Substituting L-4-(2,6-dimethoxyphenyl)phenylalanine methyl ester from Method TTT for L-4-(N,N-dimethylcarbamyloxy)phenylalanine tert-butyl ester and following the procedure described for the preparation of Example 393 yielded the title compound.

Physical data were as follows:

¹H NMR (CD₃OD): δ 7.37-7.19 (m, 5.5H), 7.09-7.02 (m, 4H), 6.94 (d, 0.5H), 6.68 (d, 2H), 4.79-4.74 (m, 0.5H), 4.69-4.65 (m, 0.5H), 3.67 (s, 3H), 3.65 (s, 3H), 3.44-3.33 (m, 1H), 3.02-2.95 (m, 4H), 2.19 (s, 1.5H), 1.85-1.71 (m, 6.5H), 1.57 (m, 4H), 1.29-1.2 (br s, 1H).

### EXAMPLE 502

### Synthesis of N-(5-(2-Fluorophenyl)pyrimidin-4-yl)-L-4-(2,6-dimethoxyphenyl)phenylalanine

Substituting L-4-(2,6-dimethoxyphenyl)phenylalanine methyl ester from Method TTT for L-4-(N,N-dimethylcarbamyloxy)phenylalanine tert-butyl ester and following the procedure described for the preparation of Example 445 yielded the title compound.

Physical data were as follows:

¹H NMR (CD₃OD): δ 8.50 (s, 1H), 8.01 (s, 1H), 7.3-7.0 (m, 9H), 6.69 (d, 2H), 5.0 (m, 1H), 3.65 (s, 6H), 3.20-3.05 (m, 2H).

¹³C NMR (CD₃OD): δ 153.2, 151.6, 147.1, 130.2, 128.6, 126.7, 126.6, 126.5, 126.4, 126.3, 123.9, 123.5, 123.2, 120.5, 120.4, 111.7, 111.4, 99.6, 59.3, 31.7.

### EXAMPLE 503

### Synthesis of N-(2-(N-Methyl-N-propyl)-5-(2-tolyl)pyrimidin-4-yl)-L-4-(2,6-dimethoxyphenyl)phenylalanine

Substituting L-4-(2,6-dimethoxyphenyl)phenylalanine methyl ester from Method TTT for L-4-(N,N-dimethylcarbamyloxy)phenylalanine tert-butyl ester and following the procedure described for the preparation of Example 436 yielded the title compound.

Physical data were as follows:

¹H NMR (CD₃OD): δ 10.30-8.80 (br, 1H), 7.68 (s, 0.5H), 7.63 (s, 0.5H), 7.40-6.60 (m, 1H), 6.15 (m, 1H), 4.70 (m, 1H), 3.68 (s, 3H), 3.66 (s, 3H), 3.80-3.00 (m, 4H), 3.07 (s, 3H), 2.12 (s, 1.5H), 2.08 (s, 1.5H), 1.61 (bs, 2H), 0.87 (bs, 3H).

### EXAMPLE 504

### Synthesis of N-(3-chloropyrazin-2-yl)-L-4-[1-(tert-butoxycarbonyl)piperidin-4-ylcarbonylamino]phenylalanine ethyl ester

### Step A: Preparation of N-(3-Chloropyrazin-2-yl)-L-4-nitrophenylalanine

4-Nitrophenylalanine (50 mm, 10.59 mg) were stirred in absolute ethanol containing 1.0 eq (1.26 g) of sodium metal. The reaction mixture was stripped to a brown solid and the sodium salt was taken up in 200 mL of butanol containing 1.0 eq (7.45 g) 2,3-dichloropyrazine. The reaction mixture was refluxed overnight and the solvent was then removed under reduced pressure. The residue was taken up in ethyl acetate and washed with water (1×), brine (1×), dried over Na₂SO₄, filtered and stripped to give 15.5 g of the title intermediate as a brown oil.

Physical data were as follows:

Analytical: MS: (+) FAB [M+H] @ M/Z 323 with 1 Cl.

### Step B: Preparation of N-(3-Chloropyrazin-2-yl)-L-4-nitrophenylalanine Ethyl Ester

The intermediate from Step A was suspended in in 300 mL of absolute ethanol. The reaction flask was placed in an ice bath and cooled to 0° C. and HCl (g) was bubbled into reaction for 15 minutes. The gas tube was replaced with a drying tube and the reaction mixture was warmed to room temperature and stirred overnight. Ethanol was stripped off under reduced pressure to afford a dark brown residue which was taken up in ethyl acetate and washed with sat. NaHCO₃ (2×), H₂O (1×), brine (1×), dried over Na₂SO₄, filtered and stripped to afford 15 g of a dark brown oil. This oil (8.0 g) was chromatographed on a silica 60 column packed in methylene chloride to provide 1.5 g (20% yield) of the title intermediate.

Physical data were as follows:

Analytical: MS: EI M⁺ @ M/Z 350 1 Cl present.

### Step C: Preparation of N-(3-Chloropyrazin-2-yl)-L-4-aminophenylalanine Ethyl Ester

The intermediate from Step B (0.75 g, 0.021 mol) was placed in a Paar hydrogenation bottle with 50 mL ethanol and 0.40 g of Pd/C catalyst. The bottle was placed on Paar shaker under 50 psi of H₂ for 3 hrs. The reaction mixture was then fitered through a sintered glass funnel (F) and the filtered catalyst was washed with ethanol. The combined filtrates were stripped to a yellow oil and the oil was taken up in ethyl acetate. A yellow precipitate formed and was filtered off. The filterate was washed with NaHCO₃ soution (1×), H₂O (1×), brine (1×), dried over Na₂SO₄, filtered and stripped to afford the title intermediate as a yellow oil (0.340 g, 55% yield)

### Step C: Preparation of N-(3-Chloropyrazin-2-yl)-L-4-[1-(tert-butoxycarbonyl)piperidin-4-ylcarbonylamino]phenylalanine Ethyl Ester

N-Boc-piperidine 4-carboxylic acid (0.253 g, 1.0 eq., 0.0011 mol) was stirred in 30 mL methylene chloride and reaction mixture was cooled to 0° C. in ice bath. HOBt (0.224 g, 1.5 eq) was added and the mixture was stirred for 10 minutes then the intermediate from step C (1 eq., 0.32 g) was added. The reaction mixture was stirred for 5 minutes and then 1,3-dicyclohexylcarbodiimide (0.25 g, 1.1 eq) was added. The reaction mixture was warmed to room temperature and stirred overnight. The reaction was then filtered and the filtrate was stripped to give a yellow solid. The solid was taken up in ethyl acetate and filtered. The ethyl acetate solution was washed with 10% citric acid (1×), H₂O (1×), brine (1×), dried over Na₂SO₄, filtered and stripped to afford a yellow oil (0.630 g; MS: EI M+@ M/Z 531 (1 chloro)). The yellow oil was chromatographed on a silica 60 column eluting with 3:1 hexane/ethyl acetate to afford 0.997 g of the title compound. This compound may also be used as an intermediate for other compounds of this invention.

Physical data were as follows:

Analytical: CHN: Theory (0.5H₂O): C, 57.71; H, 6.72; N, 12.94 Found: C, 57.79; H, 6.32; N, 12.78. MS: M⁺@ M/Z 531 (1 Chloro).

10.3 Synthesis of Compounds of Formulae VII-XX

Compounds of the present invention may be prepared as illustrated in the Scheme 16 and as described in the methods below:

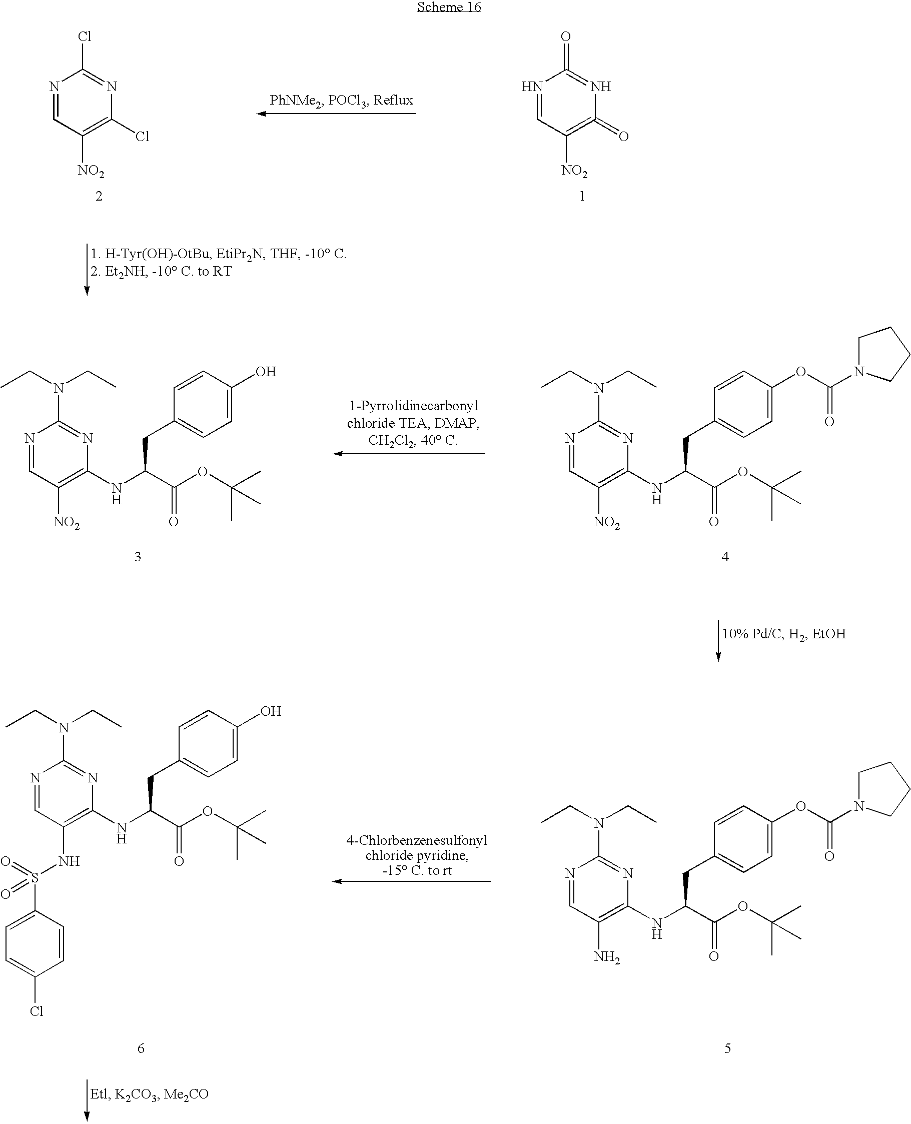

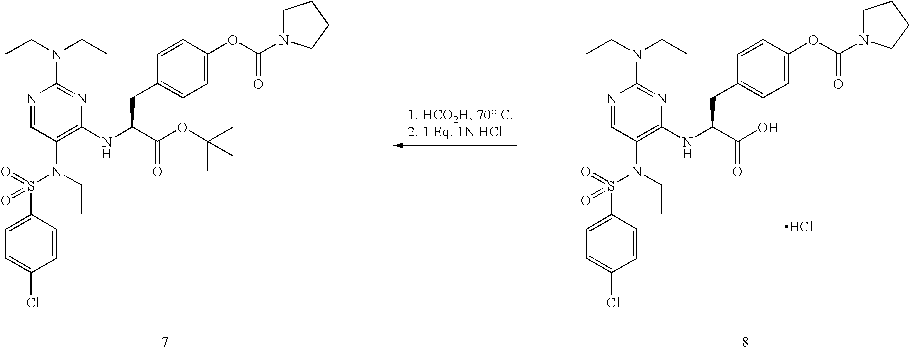

### EXAMPLE 505

### Preparation of N-(2-[N′,N′-diethylamino]-5-[N″-(4-chlorophenylsulfonyl)-N″-ethylamino]pyrimidin-4-yl)-4′-(pyrrolidin-1-ylcarbonyloxy)-L-phenylalanine

### Step 1: Preparation of 2,4-Dichloro-5-nitropyrimidine (2).

As in Scheme 16 above, 5-Nitrouracil (compound 1), was treated with phosphorous oxychloride and N,N-dimethylaniline, according to the procedure of Whittaker (J. Chem. Soc. 1951, 1565), to give compound 2. Compound 2 is also available from City Chemical (West Haven, Conn.).

### Step 2: Preparation of N-(2-1N′,N′-diethylamino]-5-nitropyrimidin-4-yl)-L-tyrosine tert-butyl ester (3)

To a solution of L-tyrosine tert-butyl ester (30.6 g, 0.129 mol) in THF (250 mL) at −10° C. was added 2,4-Dichloro-5-nitropyrimidine (25 g, 0.129 mol), keeping the temperature below 5° C. during the addition. Once the addition was complete, N,N-diisopropylethylamine (33.7 mL, 0.194 mol) was added dropwise. After stirring for 1 h at −10° C., diethylamine (66.73 mL, 0.645 mol) was added slowly, and then the reaction mixture was warmed to room temperature overnight. The reaction mixture was diluted with diethyl ether (500 mL), and the organic layer was washed with 0.2 N citric acid (3×150 mL), water (1×150 mL), and 10% K₂CO₃ (3×150 mL). The organic phase was dried (Na₂SO₄), filtered, and concentrated in vacuo to yield a yellow residue. The residue was purified by flash chromatography (20% EtOAc/hexanes on silica gel) to yield 37.39 g (67%) 3 as a yellow foam. R_{f}=0.21 (25% EtOAc/hexanes on silica gel).

### Step 3: Preparation of N-(2-[N′,N′-diethylamino]-5-nitropyrimidin-4-yl)-4′-(pyrrolidin-1-ylcarbonyloxy)-L-henylalanine tert-butyl ester (4)

To a solution of N-(2-[N′,N′-diethylamino]-5-nitropyrimidin-4-yl)-L-tyrosine tert-butyl ester (37.39 g, 0.087 mol) in CH₂Cl₂ (150 mL) was added DMAP (10.59 g, 0.087 mol). After 5 minutes triethylamine (18.19 mL, 0.131 mol) was added dropwise. Pyrrolidinecarbamoyl chloride (14.42 mL, 0.131 mol) was added dropwise, and the reaction was heated to reflux overnight. The reaction mixture was concentrated in vacuo and taken up in EtOAc (300 mL). The organic phase was washed with 0.2 N citric acid (3×150 mL), water (1×150 mL), sat. NaHCO₃ (3×150 mL), brine (1×150 mL), dried (Na₂SO₄), filtered, and concentrated in vacuo to yield 43.07 g (94%) 4 as a yellow solid. R_{f}=0.5 (50% ×EtOAc/hexanes on silica gel).

### Step 4: Preparation of N-(2-[N′,N′-diethylamino]-5-aminopyrimidin-4-yl)-4′-(pyrrolidin-1-ylcarbonyloxy)-L-phenylalanine tert-butyl ester (5)

A mixture of N-(2-[N′,N′-diethylamino]-5-nitropyrimidin-4-yl)-4′-(pyrrolidin-1-ylcarbonyloxy)-L-henylalanine tert-butyl ester (43.07 g, 0.081 mol) and 10% Pd/C (4.3 g, 10 wt % Pd) in EtOH (200 mL) was shaken under 45 psi hydrogen until TLC (50% EtOAc/hexanes on silica gel) showed 100% conversion to product (48 hours). The reaction mixture was then filtered through a Celite plug and concentrated in vacuo to yield 40.29 g (100%) 5 as a purple foam. R_{f}=0.11 (6:1 EtOAc/hexanes on silica gel).

### Step 5: Preparation of N-(2-[N′,N′-diethylamino]-5-[N″-(4-chlorophenyl-sulfonyl)amino]pyrimidin-4-yl)-4′-(pyrrolidin-1-ylcarbonyloxy)-L-phenylalanine tert-butyl ester (6)

A pyridine (160 mL) solution of N-(2-[N′,N′-diethylamino]-5-aminopyrimidin-4-yl)-4′-(pyrrolidin-1-ylcarbonyloxy)-L-phenylalanine tert-butyl ester (40.29 g, 0.081 mol) was cooled to −20° C. with a dry ice/CH₃CN bath. The mixture stirred for 30 minutes, and then p-chlorobenzenesulfonyl chloride (17.06 g, 0.081 mol) was added slowly. The reaction was stirred at −20° C. for 4 h and then allowed to warm to room temperature overnight. The reaction was diluted with EtOAc (400 mL), and the organic phase was washed with 0.2 N citric acid (3×150 mL), water (1×150 mL), sat. NaHCO₃ (3×150 μL), brine (1×150 mL), dried (Na₂SO₄), filtered, and concentrated in vacuo to yield a brown residue. The residue was purified by flash chromatography (50% EtOAc/hexanes on silica gel) to yield 43.49 g (80%) 6 as a yellow foam. R_{f}=0.35 (50% EtOAc/hexanes on silica gel).

### Step 6: Preparation of N-(2-[N′,N′-diethylamino]-5-[N″-(4-chlorophenyl-sulfonyl)-N″-ethylamino]pyrimidin-4-yl)-4′-(pyrrolidin-1-ylcarbonyloxy)-L-phenylalanine tert-butyl ester (7)

To a solution of N-(2-[N′,N′-diethylamino]-5-[N″-(4-chlorophenyl-sulfonyl)amino]pyrimidin-4-yl)-4′-(pyrrolidin-1-ylcarbonyloxy)-L-phenylalanine tert-butyl ester (42.92 g, 0.064 mol) in acetone (600 mL) was added K₂CO₃ (12.75 g, 0.096 mol), and the mixture was stirred for 1 h at room temperature. Iodoethane (7.73 mL, 0.096 mol) was then added slowly, and the reaction mixture was stirred overnight at room temperature. The reaction mixture was concentrated in vacuo, and the residue was taken up in EtOAc (300 mL). The organic phase was washed with water (2×300 mL), brine (1×100 mL), dried (Na₂SO₄), filtered, and concentrated in vacuo. The residue was purified by flash chromatography (2:1 hexanes/EtOAc on silica gel) to yield 37.36 g (85%) 7 as a white solid. R_{f}=0.53 (50% EtOAc/hexanes on silica gel).

### Step 7: Preparation of N-(2-[N′,N′-diethylamino]-5-[N″-(4-chlorophenyl-sulfonyl)-N″-ethylamino]pyrimidin-4-yl)-4′-(pyrrolidin-1-ylcarbonyloxy)-L-phenylalanine hydrochloride (8)

A formic acid (500 mL) solution of N-(2-[N′,N′-diethylamino]-5-[N″-(4-chlorophenyl-sulfonyl)-N″-ethylamino]pyrimidin-4-yl)-4′-(pyrrolidin-1-ylcarbonyloxy)-L-phenylalanine tert-butyl ester (36.21 g, 0.052 mol) was heated to 70° C. for 2 h and then concentrated in vacuo. The residue was dissolved again in formic acid (500 mL) and heated again at 70° C. for 2 h. The solution was reduced in volume by 80% and then treated with 1.0 N HCl (52 mL, 0.052 mol) followed by distilled water (100 mL). The resulting heterogeneous mixture was concentrated in vacuo. Distilled water (100 mL) was added, and the heterogeneous mixture was concentrated in vacuo. The latter steps were repeated twice to yield a wet white product. This was dried by placing under high vacuum at 40° C. (7 days) to yield 32.8 g (93%) 8, as a free-flowing white solid. R^{f}=0.25 (7/3 MeOH/H₂O+0.1% TFA, reverse phase).

### EXAMPLE 506

### Preparation of N-(2-[N′,N′-diethylamino]-5-[N″-(4-fluorophenylsulfonyl)-N″-ethylamino]pyrimidin-4-yl)-4′-(pyrrolidin 1-ylcarbonyloxy)-L-phenylalanine

Steps 1, 2, 3, 4, 6 and 7 were performed as for Example 505. Step 5 was performed using 4-fluorobenzenesulfonyl chloride in place of 4-chlorobenzenesulfonyl chloride.

### EXAMPLE 507

### Preparation of N-(2-[N′,N′-diethylamino]-5-[N″-(4-fluorophenylsulfonyl)-N″-methylamino]pyrimidin-4-yl)-4′-(pyrrolidin-1-ylcarbonyloxy)-L-phenylalanine

Steps 1, 2, 3, 4, 5 and 7 were performed as for Example 506. Step 6 was performed using dimethyl sulfate in place of ethyl iodide.

### EXAMPLE 508

### Preparation of N-(2-[N′,N′-diethylamino]-5-[N″-(4-chlorophenylsulfonyl)-N″-methylamino]pyrimidin-4-yl)-4′-(pyrrolidin-1-ylcarbonyloxy)-L-phenylalanine

Steps 1, 2, 3, 4, 5 and 7 were performed as for Example 505. Step 6 was performed using dimethyl sulfate in place of ethyl iodide.

### EXAMPLE 509

### Preparation of N-(2-[N′,N′-diethylamino]-5-[N″-(4-fluorophenylsulfonyl)-N″-methylamino]pyrimidin-4-yl)-4′-(piperidin-1-ylcarbonyloxy)-L-phenylalanine

Steps 1, 2, 4, 5, 6 and 7 were performed as for Example 507. Step 3 was performed using 1-piperidinecarbonyl chloride in place of 1-pyrrolidinecarbonyl chloride.

### EXAMPLE 510

### Preparation of N-(2-[N′,N′-diethylamino]-5-[N″-(4-fluorophenylsulfonyl)-N″-ethylamino]pyrimidin-4-yl)-4′-(piperidin-1-ylcarbonyloxy)-L-phenylalanine

Steps 1, 2, 4, 5, 6 and 7 were performed as for Example 506. Step 3 was performed using 1-piperidinecarbonyl chloride in place of 1-pyrrolidinecarbonyl chloride.

### EXAMPLE 511

### Preparation of N-(2-[N′,N′-diethylamino]-5-[N″-(4-fluorophenylsulfonyl)-N″-ethylamino]pyrimidin-4-yl)-4′-(azetidin-1-ylcarbonyloxy)-L-phenylalanine

Steps 1, 2, 4, 5, 6 and 7 were performed as for Example 506. Step 3 was performed according to the following procedure.

### Alternative Preparation of N-(2-[N′,N′-diethylamino]-5-nitropyrimidin-4-yl)-4′-(azetidin-1-ylcarbonyloxy)-L-phenylalanine tert-butyl ester

To a −15° C. stirred solution of 3 (24.9 g, 0.0578 mol) and 4-nitrophenyl chloroformate (11.7 g, 0.0578 mmol) in CH₂Cl₂ (300 mL) was added triethylamine (24.2 mL, 0.173 mol), at a rate such that the temperature of the reaction mixture did not exceed −10° C. After stirring for 20 min, azetidine (3.30 g, 0.0578 mmol) was added dropwise, and the reaction mixtures was warmed to room temperature and stirred overnight. The reaction mixture was diluted with EtOAc (100 mL) and hexanes (100 mL), and then was extracted repeatedly with 10% aqueous K₂CO₃, until no yellow color (4-nitrophenol) was seen in the aqueous phase. The organic layer was washed with brine (75 mL), dried with MgSO₄, filtered, and evaporated to yield 28.5 g (96%) N-(2-[N′,N′-diethylamino]-5-nitropyrimidin-4-yl)-4′-(azetidin-1-ylcarbonyloxy)-L-phenylalanine tert-butyl ester as a yellow solid, which was used without purification. R^{f}=0.17 (2:5 EtOAc/hexanes on silica gel).

### EXAMPLE 512

### Preparation of N-(2-[N′,N′-diethylamino]-5-[N″-(4-fluorophenylsulfonyl)-N″-methylamino]pyrimidin-4-yl)-4′-(azetidin-1-ylcarbonyloxy)-L-phenylalanine

Steps 1, 2, 3, 4, 5 and 7 were performed as for Example 511. Step 6 was performed using dimethyl sulfate in place of ethyl iodide.

### EXAMPLE 513

### Preparation of N-(2-[N′,N′-diethylamino]-5-N″-(4-chlorophenylsulfonyl)-N″-methylamino]pyrimidin-4-yl)-4′-(azetidin-1-ylcarbonyloxy)-L-phenylalanine

Steps 1, 2, 3, 4, 6 and 7 were performed as for Example 512. Step 5 was performed using 4-chlorobenzenesulfonyl chloride in place of 4-fluorobenzenesulfonyl chloride.

### EXAMPLE 514

### Preparation of N-(2-[N′,N′-diethylamino]-5-N″-(4-chlorophenylsulfonyl)-N″-ethylamino]pyrimidin-4-yl)-4′-(azetidin-1-ylcarbonyloxy)-L-phenylalanine

Steps 1, 2, 3, 4, 6 and 7 were performed as for Example 511. Step 5 was performed using 4-chlorobenzenesulfonyl chloride in place of 4-fluorobenzenesulfonyl chloride.

### EXAMPLE 515

### Preparation of N-(2-[N′,N′-diethylamino]-5-[N″-(2,4-difluorophenylsulfonyl)-N″-methylamino]pyrimidin-4-yl)-4′-(pyrrolidin-1-ylcarbonyloxy)-L-phenylalanine

Steps 1, 2, 3, 4, 6 and 7 were performed as for Example 507. Step 5 was performed using 2,4-difluorobenzenesulfonyl chloride in place of 4-fluorobenzenesulfonyl chloride.

### EXAMPLE 516

### Preparation of N-(2-[N′,N′-diethylamino]-5-[N″-(2,4-difluorophenylsulfonyl)-N″-ethylamino]pyrimidin-4-yl)-4′-(pyrrolidin-1-ylcarbonyloxy)-L-phenylalanine

Steps 1, 2, 3, 4, 6 and 7 were performed as for Example 506. Step 5 was performed using 2,4-difluorobenzenesulfonyl chloride in place of 4-fluorobenzensulfonyl chloride.

### EXAMPLE 517

### Preparation of N-(2-[N′,N′-diethylamino]-5-[N″-(2,4-difluorophenylsulfonyl)-N″-methylamino]pyrimidin-4-yl)-4′-(azetidin-1-ylcarbonyloxy)-L-phenylalanine

Steps 1, 2, 4, 5, 6 and 7 were performed as for Example 515. Step 3 was performed as for Example 511.

### EXAMPLE 518

### Preparation of N-(2-[N′,N′-diethylamino]-5-[N″-(2,4-difluorophenylsulfonyl)-N″-ethylamino]pyrimidin-4-yl)-4′-(azetidin-1-ylcarbonyloxy)-L-phenylalanine

Steps 1, 2, 4, 5, 6 and 7 were performed as for Example 516. Step 3 was performed as for Example 511.

### EXAMPLE 519

### Preparation of N-(2-[N′,N′-diethylamino]-5-[N″-(4-fluorophenylsulfonyl)-N″-propargylamino]pyrimidin-4-yl)-4′-(pyrrolidin-1-ylcarbonyloxy)-L-phenylalanine

Steps 1, 2, 3, 4, 5 and 7 were performed as for Example 506. Step 6 was performed using propargyl bromide in place of ethyl iodide.

### EXAMPLE 520

### Preparation of N-(2-[N′,N′-diethylamino]-5-[N″-(2,4-difluorophenylsulfonyl)-N″-propargylamino]pyrimidin-4-yl)-4′-(pyrrolidin-1-ylcarbonyloxy)-L-phenylalanine

Steps 1, 2, 3, 4, 5 and 7 were performed as for Example 515. Step 6 was performed using propargyl bromide in place of dimethyl sulfate.

### EXAMPLE 521

### Preparation of N-(2-[N′,N′-diethylamino]-5-[N″-(2,4-difluorophenylsulfonyl)-N″-propargylamino]pyrimidin-4-yl)-4′-(azetidin-1-ylcarbonyloxy)-L-phenylalanine

Steps 1, 2, 4, 5, 6 and 7 were performed as for Example 520. Step 3 was performed as for Example 511.

### EXAMPLE 522

### Preparation of N-(2-[N′,N′-diethylamino]-5-[N″-(4-fluorophenylsulfonyl)-N″-propargylamino]pyrimidin-4-yl)-4′-(azetidin-1-ylcarbonyloxy)-L-phenylalanine

Steps 1, 2, 3, 4, 5 and 7 were performed as for Example 511. Step 6 was performed using propargyl bromide in place of ethyl iodide.

### EXAMPLE 523

### Preparation of N-(2-[N′,N′-diethylamino]-5-[N″-(4-chlorophenylsulfonyl)-N″-propargylamino]pyrimidin-4-yl)-4′-(pyrrolidin-1-ylcarbonyloxy)-L-phenylalanine

Steps 1, 2, 3, 4, 5 and 7 were performed as for Example 505. Step 6 was performed using propargyl bromide in place of ethyl iodide.

### EXAMPLE 524

### Preparation of 2-{2-diethylamino-5-[(4-fluorobenzenesulfonyl)methylamino]pyrimidin-4-ylamino}-3-(4-dimethylcarbamoyloxyphenyl)propionic acid

General.

Flash chromatography was performed using a Biotage Flash 75L, using 800 g KP-Sil silica cartridges (32-63 μM, 60 angstrom, 500-550 m 2/g). R_{f}s are reported for analytical thin layer chromatography, using EM Science Silica Gel F(254) 250 μM thick plates for normal phase, and Watman MKC18F 200 μM thick plates for reverse phase.

### Step 1: Preparation of 2,4-Dichloro-5-nitropyrimidine.

5-Nitrouracil, was treated with phosphorous oxychloride and N,N-dimethylaniline, according to the procedure of Whittaker (J. Chem. Soc. 1951, 1565), to give the title compound, which is also available from City Chemical (West Haven, Conn.).

### Step 2: Preparation of 2-(2-diethylamino-5-nitropyrimidin-4-ylamino)-3-(4-hydroxyphenyl)propionic acid t-butyl ester

To a solution of 2-amino-3-(4-hydroxyphenyl)propionic acid, (30.6 g, 0.129 mol) in THF (250 mL) at −10° C. was added 2,4-Dichloro-5-nitropyrimidine (25 g, 0.129 mol), keeping the temperature below 5° C. during the addition. Once the addition was complete, N,N-diisopropylethylamine (33.7 mL, 0.194 mol) was added dropwise. After stirring for 1 h at −10° C., diethylamine (66.73 mL, 0.645 mol) was added slowly, and then the reaction mixture was warmed to room temperature overnight. The reaction mixture was diluted with diethyl ether (500 mL), and the organic layer was washed with 0.2 N citric acid (3×150 mL), water (1×150 mL), and 10% K₂CO₃ (3×150 mL). The organic phase was dried (Na₂SO₄), filtered, and concentrated in vacuo to yield a yellow residue. The residue was purified by flash chromatography (20% EtOAc/hexanes on silica gel) to yield 37.39 g (67%) the title compound as a yellow foam. R_{f}=0.21 (25% EtOAc/hexanes on silica gel).

### Step 3: Preparation of 2-(2-diethylamino-5-nitropyrimidin-4-ylamino)-3-(4-dimethylcarbamoyloxyphenyl)propionic acid, t-butyl ester

To a solution of 2-(2-diethylamino-5-nitropyrimidin-4-ylamino)-3-(4-hydroxy-phenyl)propionic acid, t-butyl ester (31.80 g, 0.074 mol) in CH₂Cl₂ (600 mL) was added DMAP (9.00 g, 0.074 mol). After 5 minutes triethylamine (10.23 mL, 0.074 mol) was added dropwise. N,N-dimethylcarbamyl chloride (13.83 mL, 0.110 mol) was added dropwise, and the reaction was heated to reflux overnight. The reaction mixture was concentrated in vacuo and taken up in EtOAc (1 L). The organic phase was washed with 0.5 M citric acid (3×250 mL), sat. NaHCO₃ (3×250 mL), brine (1×250 mL), dried (MgSO₄), filtered, and concentrated in vacuo to yield 37.0 g (99%) the title compound as a white solid.

### Step 4: Preparation of 2-(2-diethylamino-5-aminopyrimidin-4-ylamino)-3-(4-dimethylcarbamoyloxyphenyl)propionic acid, t-butyl ester

A mixture of 2-(2-diethylamino-5-nitropyrimidin-4-ylamino)-3-(4-dimethylcarbamoyl-oxyphenyl)propionic acid, t-butyl ester (37.0 g, 0.073 mol) and 10% Pd/C (3.8 g, 10 wt % Pd) in EtOH (250 mL) was shaken under 60 psi hydrogen until TLC (50% EtOAc/hexanes on silica gel) showed 100% conversion to product (48 hours). The reaction mixture was then filtered through a Celite plug and concentrated in vacuo to yield 32.0 g (92%) the title compound as a violet foam.

### Step 5: Preparation of 2-{2-diethylamino-5-[(4-fluorobenzenesulfonyl) amino]-pyrimidin-4-ylamino}-3-(4-dimethylcarbamoyloxyphenyl) propionic acid, t-butyl ester

A pyridine (120 mL) solution of 2-(2-diethylamino-5-aminopyrimidin-4-ylamino)-3-(4-dimethylcarbamoyloxy-phenyl)propionic acid, t-butyl ester (32.0 g, 0.067 mol) was cooled to −20° C. with a dry ice/CH₃CN bath. The mixture stirred for 30 minutes, and then p-fluorobenzenesulfonyl chloride (13.18 g, 0.067 mol) was added slowly. The reaction was stirred at −20° C. for 4.5 hrs, and then 3-dimethylaminopropyl amine (8.52 mL, 0.067 mol) was added, and then the mixture was allowed to warm to room temperature overnight. The reaction was concentrated in vacuo. The residue was taken up in EtOAc (1 L), and the organic phase was washed with 0.5 M citric acid (3×900 mL), water (1×900 mL), sat. NaHCO₃ (3×900 mL), brine (1×900 mL), dried (MgSO₄), filtered, and concentrated in vacuo to yield a brown residue. The residue was purified by flash chromatography (50% EtOAc/hexanes on silica gel) to yield 33.04 g (77%) the title compound as a yellow foam. R_{f}=0.54 (3:2 EtOAc/hexanes on silica gel).

### Step 6: Preparation of 2-{2-diethylamino-5-[(4-fluorobenzenesulfonyl) methylamino]-pyrimidin-4-ylamino}-3-(4-dimethylcarbamoyloxyphenyl) propionic acid, t-butyl ester

To a solution of 2-{2-diethylamino-5-[(4-fluorobenzenesulfonyl)amino]-pyrimidin-4-ylamino}-3-(4-dimethyl-carbamoyloxyphenyl)propionic acid, t-butyl ester (33.04 g, 0.052 mol) in acetone (510 mL) was added K₂CO₃ (8.69 g, 0.063 mol), and the mixture was stirred for 10 min at room temperature. Dimethyl sulfate (5.95 mL, 0.063 mol) was then added slowly, and the reaction mixture was stirred overnight at room temperature. The reaction mixture was concentrated in vacuo, and the residue was taken up in EtOAc (600 mL). The organic phase was washed with water (2×400 mL), brine (2×400 mL), dried (MgSO₄), filtered, and concentrated in vacuo. The residue was purified by flash chromatography (2:1 hexanes/EtOAc on silica gel) to yield 28.69 g (85%) the title compound as a white solid.

### Step 7: Preparation of 2-{2-diethylamino-5-[(4-fluorobenzenesulfonyl) methylamino]pyrimidin-4-ylamino}-3-(4-dimethylcarbamoyloxyphenyl) propionic acid, hydrochloride

A formic acid (500 mL) solution of 2-{2-diethylamino-5-[(4-fluorobenzenesulfonyl) methylamino]-pyrimidin-4-ylamino}-3-(4-dimethylcarbamoyloxyphenyl) propionic acid, t-butyl ester (28.69 g, 0.044 mol) was heated to 70° C. for 2 h, and then concentrated in vacuo. The residue was dissolved again in formic acid (500 mL), and then heated again at 70° C. for 2 h, and then concentrated again in vacuo. The residue was dissolved again in formic acid (500 mL), and then heated again at 70° C. for 1 h. The solution was reduced in volume by 90%, and then treated with 1.0 M HCl (44 mL, 0.044 mol) and distilled water (490 mL). The resulting homogeneous solution was concentrated in vacuo, and then distilled water (100 mL) was added, and the homogenous solution was lyophilized over 14 days to yield 26.76 g (96%) the title compound, as a white solid.

### EXAMPLE 525

### Preparation of 2-{2-diethylamino-5-[(4-chlorobenzenesulfonyl)methylamino]pyrimidin-4-ylamino}-3-(4-dimethylcarbamoyloxyphenyl)propionic acid

Steps 1, 2, 3, 4, 6 and 7 were performed as for Example 524. Step 5 was performed using 4-chlorobenzenesulfonyl chloride in place of 4-fluorobenzenesulfonyl chloride.

### EXAMPLE 526

### Preparation of 2-{2-diethylamino-5-[(3,4-difluorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 3,4-difluorobenzenesulfonyl chloride in place of 4-fluorobenzenesulfonyl chloride.

### EXAMPLE 527

### Preparation of 2-{2-diethylamino-5-[(3,4-dichlorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 3,4-dichlorobenzenesulfonyl chloride in place of 4-fluorobenzenesulfonyl chloride.

### EXAMPLE 528

### Preparation of 2-{2-diethylamino-5-[(benzenesulfonyl)methylamino]pyrimidin-4-ylamino}-3-(4-dimethylcarbamoyloxyphenyl)propionic acid

Steps 1, 2, 3, 4, 6 and 7 were performed as for Example 524. Step 5 was performed using benzenesulfonyl chloride in place of 4-fluorobenzenesulfonyl chloride.

### EXAMPLE 529

### Preparation of 2-{2-diethylamino-5-[(2-fluorobenzenesulfonyl)methylamino]pyrimidin-4-ylamino}-3-(4-dimethylcarbamoyloxyphenyl)propionic acid

Steps 1, 2, 3, 4, 6 and 7 were performed as for Example 524. Step 5 was performed using 2-fluorobenzenesulfonyl chloride in place of 4-fluorobenzenesulfonyl chloride.

### EXAMPLE 530

### Preparation of 2-{2-diethylamino-5-[(3-fluorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 3-fluorobenzenesulfonyl chloride in place of 4-fluorobenzenesulfonyl chloride.

### EXAMPLE 531

### Preparation of 2-{2-diethylamino-5-[(4-fluorobenzenesulfonyl) isopropylamino]pyrimidin-4-ylamino}-3-(4-dimethylcarbamoyloxyphenyl) propionic acid

Steps 1, 2 and 3 were performed as for Example 524. Thereafter, Steps 4 and 6 were accomplished in one pot, according to the following procedure. Thereafter,

Steps 5 and 7 were performed as for Example 524. Alternative one-pot procedure for the preparation of 2-(2-diethylamino-5-isopropylaminopyrimidin-4-yl)-3-(4-dimethylcarbamoyloxyphenyl) propionic acid, t-butyl ester.

A mixture of 2-(2-diethylamino-5-nitropyrimidin-4-ylamino)-3-(4-dimethylcarbamoyloxyphenyl)propionic acid, t-butyl ester (5.0 g, 0.010 mol), glacial acetic acid (10 drops), acetone (2.19 μL, 0.030 mol), and platinum oxide (0.250 g, 5 wt %) in EtOH (15 mL) was hydrogenated at 45 psi hydrogen until TLC (50% EtOAc/hexanes) showed 100% conversion to product (20 hours). The reaction mixture was then filtered through a Celite plug and concentrated in vacuo to yield a brown residue. The residue was purified by flash chromatography (4:1 EtOAc/hexanes) to yield 3.54 g (70%) 9 as a purple foam.

### EXAMPLE 532

### Preparation of 2-{2-diethylamino-5-[(4-fluorobenzenesulfonyl)ethylamino]pyrimidin-4-ylamino}-3-(4-dimethylcarbamoyloxyphenyl)propionic acid

Steps 1, 2, 3, 4, 5 and 7 were performed as for Example 524. Step 6 was performed using ethyl iodide in place of dimethyl sulfate.

### EXAMPLE 533

### Preparation of 2-{2-diethylamino-5-[(3,4-difluorobenzenesulfonyl) isopropylamino]pyrimidin-4-ylamino}-3-(4-dimethylcarbamoyloxyphenyl) propionic acid

Steps 1, 2, 3, 4, 6 and 7 were performed as for Example 531. Step 5 was performed using 3,4-difluorobenzenesulfonyl chloride in place of 4-fluorobenzenesulfonyl chloride.

### EXAMPLE 534

### Preparation of 2-{2-diethylamino-5-[(4-chlorobenzenesulfonyl) isopropylamino]pyrimidin-4-ylamino}-3-(4-dimethylcarbamoyloxyphenyl) propionic acid

Steps 1, 2, 3, 4, 6 and 7 were performed as for Example 531. Step 5 was performed using 4-chlorobenzenesulfonyl chloride in place of 4-fluorobenzenesulfonyl chloride.

### EXAMPLE 535

### Preparation of 2-{2-diethylamino-5-[(3,4-difluorobenzenesulfonyl) ethylamino]pyrimidin-4-ylamino}-3-(4-dimethylcarbamoyloxyphenyl) propionic acid

Steps 1, 2, 3, 4, 6 and 7 were performed as for Example 532. Step 5 was performed using 3,4-difluorobenzensulfonyl chloride in place of 4-fluorobenzenesulfonyl chloride.

### EXAMPLE 536

### Preparation of 2-{2-diethylamino-5-[(4-chlorobenzenesulfonyl)ethylamino]pyrimidin-4-ylamino}-3-(4-dimethylcarbamoyloxyphenyl)propionic acid

Steps 1, 2, 3, 4, 6 and 7 were performed as for Example 532. Step 5 was performed using 4-chlorobenzenensulfonyl chloride in place of 4-fluorobenzenesulfonyl chloride.

### EXAMPLE 537

### Preparation of 2-{2-diethylamino-5-[(4-fluorobenzenesulfonyl) cylclopropylmethylamino]pyrimidin-4-ylamino}-3-(4-dimethylcarbamoyl-oxyphenyl)propionic acid

Steps 1, 2, 3, 4, 5 and 7 were performed as for Example 524. Step 6 was performed using bromomethylcyclopropane and cesium carbonate in place of dimethyl sulfate and potassium carbonate.

### EXAMPLE 538

### Preparation of 2-{2-diethylamino-5-[(3,5-difluorobenzenesulfonyl) methylamino]pyrimidin-4-ylamino}-3-(4-dimethylcarbamoyloxypheyl) propionic acid

Steps 1, 2, 3, 4, 6 and 7 were performed as for Example 524. Step 5 was performed using 3,5-difluorobenzenesulfonyl chloride in place of 4-fluorobenzenesulfonyl chloride.

### EXAMPLE 539

### Preparation of 2-{2-diethylamino-5-[(3,5-difluorobenzenesulfonyl) ethylamino]pyrimidin-4-ylamino}-3-(4-dimethylcarbamoyloxyphenyl) propionic acid

Steps 1, 2, 3, 4, 5 and 7 were performed as for Example 538. Step 6 was performed using ethyl iodide in place of dimethyl sulfate.

### EXAMPLE 540

### Preparation of 2-{2-diethylamino-5-[(2,4-difluorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 2,4-difluorobenzenesulfonyl chloride in place of 4-fluorobenzenesulfonyl chloride.

### EXAMPLE 541

### Preparation of 2-{2-diethylamino-5-[(2,4-difluorobenzenesulfonyl) ethylamino]pyrimidin-4-ylamino}-3-(4-dimethylcarbamoyloxyphenyl) propionic acid

Steps 1, 2, 3, 4, 5 and 7 were performed as for Example 540. Step 6 was performed using ethyl iodide in place of dimethyl sulfate.

### EXAMPLE 542

### Preparation of 2-{2-diethylamino-5-[(3,5-dichlorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 3,5-dichlorobenzenesulfonyl chloride in place of 4-fluorobenzenesulfonyl chloride.

### EXAMPLE 543

### Preparation of 2-{2-diethylamino-5-[(3,5-dichlorobenzenesulfonyl) ethylamino]pyrimidin-4-ylamino}-3-(4-dimethylcarbamoyloxyphenyl) propionic acid

Steps 1, 2, 3, 4, 5 and 7 were performed as for Example 542. Step 6 was performed using ethyl iodide in place of dimethyl sulfate.

### EXAMPLE 544

### Preparation of 2-{2-diethylamino-5-[(4-fluorobenzenesulfonyl)-n-propylamino]pyrimidin-4-ylamino}-3-(4-dimethylcarbamoyloxyphenyl) propionic acid

Steps 1, 2, 3, 4, 5 and 7 were performed as for Example 524. Step 6 was performed using 1-propyl iodide in place of dimethyl sulfate.

### EXAMPLE 545

### Preparation of 2-{2-diethylamino-5-[(4-fluorobenzenesulfonyl)allylamino]pyrimidin-4-ylamino}-3-(4-dimethylcarbamoyloxyphenyl)propionic acid

Steps 1, 2, 3, 4, 5 and 7 were performed as for Example 524. Step 6 was performed using allyl bromide in place of dimethyl sulfate.

### EXAMPLE 546

### Preparation of 2-{2-diethylamino-5-[(4-fluorobenzenesulfonyl)isobotylamino]pyrimidin-4-ylamino}-3-(4-dimethylcarbamoyloxyphenyl)propionic acid

Steps 1, 2, 3, 4, 5 and 7 were performed as for Example 524. Step 6 was performed using isobutyl iodide in place of dimethyl sulfate.

### EXAMPLE 547

### Preparation of 2-{2-diethylamino-5-[(4-fluorobenzenesulfonyl)-n-butylamino]pyrimidin-4-ylamino}-3-(4-dimethylcarbamoyloxyphenyl)propionic acid

Steps 1, 2, 3, 4, 5 and 7 were performed as for Example 524. Step 6 was performed using 1-butyl iodide in place of dimethyl sulfate.

### EXAMPLE 548

### Preparation of 2-{2-diethylamino-5-[(2,5-difluorobenzenesulfonyl) methylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 2,6-difluorobenzenesulfonyl chloride in place of 4-fluorobenzenesulfonyl chloride.

### EXAMPLE 549

### Preparation of 2-{2-diethylamino-5-[(2,3-difluorobenzenesulfonyl) ethylamino]pyrimidin-4-ylamino}-3-(4-dimethylcarbamoyloxyphenyl) propionic acid

Steps 1, 2, 3, 4, 6 and 7 were performed as for Example 524. Step 5 was performed using 2,3-difluorobenzenesulfonyl chloride in place of 4-fluorobenzenesulfonyl chloride. 2,3-Difluorobenzenesulfonyl chloride was prepared by the following procedure. Preparation of 2,3-Difluorobenzenesulfonyl Chloride.

The following procedure was executed using two flasks. In the first flask, 2,3-difluoroaniline (2.0 g, 0.015 mol) was dissolved in concentrated HCl (15.9 mL), and the resulting solution was cooled to −5° C., using an ice/NaCl bath. A solution of sodium nitrite (1.18 g, 0.017 mol) in distilled water (13.6 mL) was added in portions with stirring, while maintaining the temperature below 0° C., and the mixture was stirred for 10 min. In the second flask, thionyl chloride (5.08 mL, 0.069 mol) was added dropwise to distilled water (30.6 mL), which had been pre-cooled to −5° C., using an ice/NaCl bath. The resulting solution was allowed to warm to room temperature, and then Cu(I)Cl (0.08 g, 0.77 mmol) was added, and then the reaction mixture was re-cooled to −5° C. With continued cooling and stirring, the contents of the first flask were added in 2 mL portions to the contents of the second flask, and the mixture was stirred for 30 min, during which time a precipitate formed. The precipitate was isolated by filtration, rinsed with cold water, and stored under vacuum to give 3.25 g (98%) 10 as a white solid.

### EXAMPLE 550

### Preparation of 2-{2-Diethylamino-5-[(4-fluorobenzenesulfonyl) propargylamino]pyrimidin-4-ylamino}-3-(4-dimethylcarbamoyloxyphenyl) propionic acid

Steps 1, 2, 3, 4, 5 and 7 were performed as for Example 524. Step 6 was performed using propargyl bromide in place of dimethyl sulfate.

### EXAMPLE 551

### Preparation of 2-{2-Diethylamino-5-[(2,4-difluorobenzenesulfonyl)propargylamino]pyrimidin-4-ylamino}-3-(4-dimethylcarbamoyloxyphenyl)propionic acid

Steps 1, 2, 3, 4, 5 and 7 were performed as for Example 540. Step 6 was performed using propargyl bromide in place of dimethyl sulfate.

10.4 Synthesis of Compounds of Formulae XXI and XXIa

The following Methods may be used to prepare the compounds of this invention.

### Method A

### Methyl Ester Preparation Procedure

Amino acid methyl esters can be prepared using the method of Brenner and Huber Helv. Chim. Acta 1953, 36, 1109.

### Method B

### BOP Coupling Procedure

The desired dipeptide ester was prepared by the reaction of a carboxylic acid (1 equivalent) with the appropriate amino acid ester or amino acid ester hydrochloride (1 equivalent), benzotriazol-1-yloxy-tris(dimethylamino)phosphonium hexafluorophosphate [BOP] (2.0 equivalent), triethylamine (1.1 equivalent), and DMF. The reaction mixture was stirred at room temperature overnight. The crude product is purified flash chromatography to afford the dipeptide ester.

### Method C

### Hydrogenation Procedure I

Hydrogenation was performed using 10% palladium on carbon (10% by weight) in methanol at 30 psi overnight. The mixture was filtered through a pad of Celite and the filtrate concentrated to yield the desired compound.

### Method D

### Hydrolysis Procedure I

To a chilled (0° C.) THF/H₂O solution (2:1, 5-10 mL) of the appropriate ester was added LiOH (or NaOH) (0.95 equivalents). The temperature was maintained at 0° C. and the reaction was complete in 1-3 hours. The reaction mixture was extracted with ethyl acetate and the aqueous phase was lyophilized resulting in the desired carboxylate salt.

### Method E

### Ester Hydrolysis Procedure II

To a chilled (0° C.) THF/H₂O solution (2:1, 5-10 mL) of the appropriate ester was added LiOH (1.1 equivalents). The temperature was maintained at 0° C. and the reaction was complete in 1-3 hours. The reaction mixture was concentrated and the residue was taken up into H₂O and the pH adjusted to 2-3 with aqueous HCl.

The product was extracted with ethyl acetate and the combined organic phase was washed with brine, dried over MgSO₄, filtered and concentrated to yield the desired acid.

### Method F

### Ester Hydrolysis Procedure III

The appropriate ester was dissolved in dioxane/H₂O (1:1) and 0.9 equivalents of 0.5 N NaOH was added. The reaction was stirred for 3-16 hours and then concentrated. The resulting residue was dissolved in H₂O and extracted with ethyl acetate. The aqueous phase was lyophilized to yield the desired carboxylate sodium salt.

### Method G

### BOC Removal Procedure

Anhydrous hydrochloride (HCl) gas was bubbled through a methanolic solution of the appropriate Boc-amino acid ester at 0° C. for 15 minutes and the reaction mixture was stirred for three hours. The solution was concentrated to a syrup and dissolved in Et₂O and reconcentrated. This procedure was repeated and the resulting solid was placed under high vacuum overnight.

### Method H

### tert-Butyl Ester Hydrolysis Procedure I

The tert-butyl ester was dissolved in CH₂Cl₂ and treated with TFA. The reaction was complete in 1-3 hr at which time the reaction mixture was concentrated and the residue dissolved in H₂O and lyophilized to yield the desired acid.

### Method I

### EDC Coupling Procedure I

To a CH₂Cl₂ solution (5-20 mL) of a carboxylic acid (1 equivalent), the appropriate amino acid ester hydrochloride (1 equivalent), N-methylmorpholine (1,1-2.2 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight.

The reaction mixture was poured into H₂O and the organic phase was washed with sat. NaHCO₃, brine, dried (MgSO₄ or Na₂SO₄), filtered and concentrated. The crude product was purified by column chromatography.

### Method J

### EDC Coupling Procedure II

To a DMF solution (5-20 mL) of a carboxylic acid (1 equivalent), the appropriated amino acid ester hydrochloride (1 equivalent), Et₃N (1.1 equivalents) and 1-hydroxybenzotriazole (2 equivalents) were mixed, placed in an ice bath and 1-(3-dimethylaminopropyl)-3-ethyl carbodiimide (1.1 equivalents) added. The reaction was allowed to rise to room temperature and stirred overnight. The reaction mixture was partitioned between EtOAc and H₂O and the organic phase washed with 0.2 N citric acid, H₂O, sat. NaHCO₃, brine, dried (MgSO₄ or Na₂SO₄), filtered and concentrated. The crude product was purified by column chromatography or preparative TLC.

### Method K

### tert-Butyl Ester Hydrolysis Procedure II

The tert-butyl ester was dissolved in CH₂Cl₂ (5 mL) and treated with TFA (5 mL). The reaction was complete in 1-3 hours at which time the reaction mixture was concentrated and the residue dissolved in H₂O and concentrated. The residue was redissolved in H₂O and lyophilized to yield the desired product.

### Method L

### Carbamate Formation Procedure I

Into a reaction vial were combined 15.2 mmol, 1.0 eq. of the starting hydroxy compound (typically a tyrosine derivative) and 1.86 g (15.2 mmol, 1.0 eq) DMAP. Methylene chloride (50 mL), triethylamine (2.12 mL, 1.54 g, 15.2 mmol, 1.0 eq), and dimethylcarbamyl chloride (1.68 mL, 1.96 g, 18.2 mmol, 1.2 eq) were then added. The vial was capped tightly, and the reaction solution swirled to obtain a homogeneous solution. The reaction solution was then heated to 40° C. After 48 h, TLC of the resulting colorless solution indicated complete conversion. The work-up of the reaction solution was as follows: 50 mL EtOAc and 50 mL hexanes was added to the reaction mixture, and the resulting mixture was washed with 0.5 M citric acid (3×50 mL), water (2×50 mL), 10% K₂CO₃ (2×50 mL), and sat. NaCl (1×50 mL); dried with MgSO₄, filtered and evaporated to afford the desired compound.

### Method M

### Carbamate Formation Procedure II

Into a reaction vial were combined 84.34 mmol (1.0 eq) of the starting hydroxy compound (typically a tyrosine derivative) and 17.0 g (84.34 mmol, 1.0 eq) 4-nitrophenyl chloroformate. Methylene chloride (700 mL) was added and the vial was capped with a septum. A nitrogen line was attached and the vial was immersed in a 4:1 water/ethanol dry ice slurry with stirring to cool to −15° C. Triethylamine (29.38 mL, 21.33 g, 210.81 mmol, 2.5 eq) was added over five minutes with stirring and the stirring was continued at −10 to −15° C. for 1 h. N-Methyl piperazine (9.35 mL, 8.45 g, 84.34 mmol, 1.0 eq) was added over three minutes with stirring and stirring was continued overnight while warming to room temperature. The reaction mixture was diluted with 700 mL hexanes and the resulting mixture was washed repeatedly with 10% K₂CO₃, until no yellow color (from 4-nitrophenol) is observed in the aqueous layer. The mixture was then washed with sat. NaCl, dried over anhydrous MgSO₄, filtered and evaporated. The residue was dissolved in 500 mL of ethanol and evaporated to remove triethylamine. The residue was again dissolved in 500 mL of ethanol and evaporated to remove triethylamine. The residue was then dissolved in 400 mL of ethanol and 600 mL of water was added with stirring to precipitate a solid or oil. If an oil if formed, the oil is stirred vigorously to induce it to solidify. The solid is then isolated by filtration. Dissolution, precipitation, and filtration are repeated once and the resulting solid is rinsed with water to remove traces of yellow color. The solid is then subjected to high vacuum until the mass remains constant thereby affording the desired carbamyloxy compound.

### Method N

### tert-Butyl Ester Hydrolysis Procedure III

A solution of the tert-butyl ester (typically 0.95 mmol) in 25 mL of formic acid was stirred at 25° C. for 24 hr. The solvent was removed and the residue was washed with diethyl ether (3×) to afford the desired product as a white solid.

### EXAMPLE 552

### Synthesis of N-Benzyl-L-pyroglutamyl-L-phenylalanine

### Step A—Preparation of N-Benzyl-L-pyroglutamic Acid Ethyl Ester

Ethyl (S)-(+)-2-pyrrolidone-5-carboxylate (1 g, 6.36 mmol) and benzyl bromide (0.76 mL, 6.36 mmol) were placed in dry THF (30 mL). The reaction mixture was stirred and cooled to 0° C. A 1M solultion of tert-BuOK was added dropwise (6.36 mL, 6.36 mmol) and the reaction was stirred for an additional 0.5 h at 0° C. and allowed to come to room temperate where it was stirred for 24 hours under N₂. The reaction was then dissolved into a 1:1 mixture of H₂O/EtOAc. The organic layer was washed with 1M HCl, H₂O and brine, and then dried over MgSO₄ to afford N-benzyl-L-pyroglutamic acid ethyl ester an oil.

### Step B—Preparation of N-Benzyl-L-pyroglutamic Acid

The ester from Step A was then hydrolyzed using the procedure described in Method F to afford N-benzyl-L-pyroglutamic acid.

### Step C—Preparation of N-Benzyl-L-pyroglutamyl-L-phenylalanine Ethyl Ester

The product from Step B was then coupled with L-phenylalanine ethyl ester using the procedure described in Method B (with substitution of N-methylmorpholine for triethylamine) to afford N-Benzyl-L-pyroglutamyl-L-phenylalanine ethyl ester.

NMR data was as follows:

¹H NMR (CDCl₃): δ 7.29 (m, 6H), 7.10 (m, 4H), 6.28 (brd, 1H), 5.13 (d, 1H), 4.90 (m, 1H), 4.19 (q, 2H), 3.77 (m, 2H), 3.29-2.98 (m, 2H), 2.37 (m, 2H), 2.16 (m, 1H), 1.82 (m, 1H), 1.28 (t, 3H).

¹³C NMR (CDCl₃): δ 176.18 171.84, 171.49, 136.32, 136.19, 129.67, 129.37, 129.31, 129.25, 129.01, 128.41, 127.87, 62.38, 60.58, 53.24, 45.89, 38.24, 30.12, 23.84, 14.74.

### Step D—Preparation of N-Benzyl-L-pyroglutamyl-L-phenylalanine

The title compound was prepared by hydrolysis of the product from Step C using the procedure described in Method F.

NMR data was as follows:

¹H NMR (CDCl₃): δ 7.28 (m, 6H), 7.11 (m, 4H), 6.67 (brd, 1H), 5.07 (d, 1H), 4.97 (m, 1H), 3.83 (m, 1H), 3.71 (d, 1H), 3.30 (m, 1H), 3.00 (m, 1H), 2.38 (m, 2H), 2.16 (m, 1H), 1.73 (m, 1H).

### EXAMPLE 553

### Synthesis of N-Benzyloxycarbonyl-L-pyroglutamyl-L-phenylalanine

N-Benzyloxycarbonyl-L-pyroglutamyl-L-phenylalanine tert-butyl ester was prepared from the appropriate starting materials using the procedure described in Method B. The title compound was then prepared using the procedure described in Method D.

NMR data was as follows:

¹H NMR (CDCl₃): δ 7.40-6.92 (m, 1H), 5.19 (s, 2H), 4.93 (m, 1H), 4.55 (m, 1H), 3.25-2.89 (m, 2H), 2.42 (m, 2H), 2.16 (m, 1H), 1.94 (m, 1H).

¹³C NMR (CDCl₃): δ 175.1 174.6 171.0, 151.8, 136.4, 135.3, 129.9, 129.2, 129.1, 129.1, 128.8, 127.7, 69.2, 60.6, 53.4, 38.0, 31.8, 22.9.

### EXAMPLE 554

### Synthesis of N-Benzyl-L-pyroglutamyl-L-4-(phenylcarbonylamino)phenylalanine

The title compound was prepared from the appropriate starting materials using the procedures described in Examples 552 and 555.

NMR data was as follows:

¹H NMR (DMSO-d₆): δ 10.24 (s, 1H), 8.53 (d, 1H), 7.93 (d, 2H), 7.74 (d, 2H), 7.64-7.49 (m, 3H), 7.35-7.16 (m, 5H), 7.05 (d, 2H), 4.78 (d, 1H), 4.54 (m, 1H), 3.88 (m, 1H), 3.20-2.78 (m, 2H), 2.22 (m, 2H), 2.12 (m, 1H), 1.73 (m, 1H).

¹³C NMR (DMSO-d₆): δ 175.0, 172.9, 171.3, 165.8, 137.7, 136.5, 135.0, 132.7, 131.5, 129.2, 128.5, 128.4, 127.8, 127.6, 127.3, 58.8, 53.2, 44.2, 36.0, 29.3, 22.3.

### EXAMPLE 555

### Synthesis of N-(3,4-Dichlorobenzyl)-L-pyroglutamyl-L-4-(phenylcarbonylamino)phenylalanine

### Step A—Preparation of N-(3,4-Dichlorobenzyl)-L-pyroglutamyl-L-4-aminophenylalanine Methyl Ester

N-(3,4-Dichlorobenzyl)-L-pyroglutamyl-L-4-aminophenylalanine methyl ester was prepared from the appropriate starting materials using the procedures described in Methods B and C.

### Step B—Preparation of N-(3,4-Dichlorobenzyl)-L-pyroglutamyl-L-4-(phenylcarbonylamino)phenylalanine Methyl Ester

The ester from Step A (230 mg, 0.495 mmol) was then placed in pyridine and benzoyl chloride (63.2 mL, 0.545 mmol) was added dropwise and the reaction was stirred for 2 hours. The resulting mixture was evaporated to dryness and taken up in EtOAc. The organic layer was washed with H₂O, 1 M HCl, brine, and dried over MgSO₄ to give N-(3,4-dichlorobenzyl)-L-pyroglutamyl-L-4-(phenylcarbonylamino)-phenylalanine methyl ester as a white solid.

### Step C—Preparation of N-(3,4-Dichlorobenzyl)-L-pyroglutamyl-L-4-(phenylcarbonylamino)phenylalanine Methyl Ester

The title compound was prepared by hydrolyzing the product from Step B using the procedure described in Method F.

NMR data was as follows:

¹H NMR (DMSO-d₆): δ 10.22 (s, 1H), 8.56 (brd, 1H), 7.93 (d, 2H), 7.68 (d, 2H), 7.56 (m, 4H), 7.31 (s, 1H), 7.20 (d, 2H), 7.00 (d, 1H), 4.60 (d, 1H), 4.53 (m, 1H), 3.96 (m, 1H), 3.42 (d, 1H), 3.16-2.79 (m, 2H), 2.32 (m, 2H), 2.19 (m, 1H), 1.79 (m, 1H).

¹³C NMR (DMSO-d₆): δ 175.4, 173.0, 171.1, 166.0, 138.1, 138.0, 135.4, 133.1, 132.0, 131.5, 130.9, 130.1, 129.8, 129.1, 128.4, 128.3, 127.6, 120.4, 59.4, 53.4, 43.7, 36.5, 29.6, 22.1.

### EXAMPLE 556

### Synthesis of N-(3-Chlorobenzyl)-L-pyroglutamyl-L-4-(phenylcarbonylamino)phenylalanine Methyl Ester

The title compound was prepared from the appropriate starting materials using the procedure described in Example 555.

NMR data was as follows:

¹H NMR (DMSO-d₆): δ 10.23 (s, 1H), 8.67 (d, 1H), 7.92 (d, 2H), 7.70 (d, 2H), 7.52 (m, 3H), 7.31 (m, 2H), 7.19 (m, 3H), 6.98 (m, 2H), 4.68 (d, 1H), 4.58 (m, 1H), 3.93 (m, 1H), 3.65 (s, 3H), 3.41 (d, 1H), 3.11-2.82 (m, 2H), 2.30 (m, 2H), 2.15 (m, 1H), 1.77 (m, 1H).

### EXAMPLE 557

### Synthesis of N-(3-Chlorobenzyl)-L-pyroglutamyl-L-4-(phenylcarbonylamino)phenylalanine

The title compound was prepared from the product of Example 556 using the procedure described in Method F.

NMR data was as follows:

¹H NMR (DMSO-d₆): δ 10.23 (s, 1H), 8.67 (d, 1H), 7.92 (d, 2H), 7.70 (d, 2H), 7.52 (m, 3H), 7.31 (m, 2H), 7.19 (m, 3H), 6.98 (m, 2H), 4.68 (d, 1H), 4.58 (m, 1H), 3.93 (m, 1H), 3.41 (d, 1H), 3.11-2.82 (m, 2H), 2.30 (m, 2H), 2.15 (m, 1H), 1.77 (m, 1H).

### EXAMPLE 558

### Synthesis of N-(4-Chlorobenzyl)-L-pyroglutamyl-L-4-(phenylcarbonylamino)phenylalanine

The title compound was prepared from the product of Example 559 using the procedure described in Method F.

NMR data was as follows:

¹H NMR (DMSO-d₆): δ 10.25 (s, 1H), 8.51 (d, 1H), 7.93 (d, 2H), 7.72 (d, 2H), 7.54 (m, 3H), 7.33 (d, 2H), 7.20 (d, 2H), 7.01 (d, 2H), 4.67 (d, 1H), 4.52 (m, 1H), 3.85 (m, 1H), 3.15-2.77 (m, 2H), 2.30 (m, 2H), 2.11 (m, 1H), 1.76 (m, 1H).

### EXAMPLE 559

### Synthesis of N-(4-Chlorobenzyl)-L-pyroglutamyl-L-4-(phenylcarbonylamino)phenylalanine Methyl Ester

The title compound was prepared from the appropriate starting materials using the procedure described in Example 555.

NMR data was as follows:

¹H NMR (DMSO-d₆): δ 10.25 (s, 1H), 8.51 (d, 1H), 7.93 (d, 2H), 7.72 (d, 2H), 7.54 (m, 3H), 7.33 (d, 2H), 7.20 (d, 2H), 7.01 (d, 2H), 4.67 (d, 1H), 4.52 (m, 1H), 3.85 (m, 1H), 3.65 (s, 3H), 3.15-2.77 (m, 2H), 2.30 (m, 2H), 2.11 (m, 1H), 1.76 (m, 1H).

### EXAMPLE 560

### Synthesis of N-(4-Methylbenzyl)-L-pyroglutamyl-L-(4-phenylcarbonylamino)phenylalanine Methyl Ester

The title compound was prepared from the appropriate starting materials using the procedure described in Example 555.

NMR data was as follows:

¹H NMR (DMSO-d₆): δ 10.25 (s, 1H), 8.59 (d, 1H), 7.94 (d, 2H), 7.74 (d, 2H), 7.55 (m, 3H), 7.20 (d, 2H), 7.09 (d, 2H), 6.89 (d, 2H), 4.73 (d, 1H), 4.60 (m, 1H), 3.82 (m, 1H), 3.66 (s, 3H), 3.32 (d, 1H), 3.13-2.81 (m, 2H), 2.30 (m, 2H), 2.24 (s, 3H), 2.10 (m, 1H), 1.73 (m, 1H).

### EXAMPLE 561

### Synthesis of N-(4-Methylbenzyl)-L-pyroglutamyl-L-4-(phenylcarbonylamino)phenylalanine

The title compound was prepared from the product of Example 560 using the procedure described in Method F.

NMR data was as follows:

¹H NMR (DMSO-d₆): δ 10.25 (s, 1H), 8.59 (d, 1H), 7.94 (d, 2H), 7.74 (d, 2H), 7.55 (m, 3H), 7.20 (d, 2H), 7.09 (d, 2H), 6.89 (d, 2H), 4.73 (d, 1H), 4.60 (m, 1H), 3.82 (m, 1H), 3.32 (d, 1H), 3.13-2.81 (m, 2H), 2.30 (m, 2H), 2.24 (s, 3H), 2.10 (m, 1H), 1.73 (m, 1H).

### EXAMPLE 562

### Synthesis of N-(4-Methoxybenzyl)-L-pyroglutamyl-L-4-(phenylcarbonylamino)phenylalanine Methyl Ester

The title compound was prepared from the appropriate starting materials using the procedures described in Example 555.

NMR data was as follows:

¹H NMR (DMSO-d₆): δ 10.25 (s, 1H), 8.59 (d, 1H), 7.94 (d, 2H), 7.74 (d, 2H), 7.55 (m, 3H), 7.42 (d, 2H), 6.92 (d, 2H), 6.83 (d, 2H), 4.73 (d, 1H), 4.60 (m, 1H), 3.83 (m, 1H), 3.70 (s, 3H), 3.65 (s, 3H), 3.32 (d, 1H), 3.16-2.81 (m, 2H), 2.30 (m, 2H), 2.10 (m, 1H), 1.73 (m, 1H).

¹³C NMR (DMSO-d₆): δ 174.8, 172.2, 171.6, 165.9, 158.9, 138.2, 136.4, 132.7, 131.9, 129.6, 129.6, 128.8, 128.6, 128.0, 120.4, 114.3, 58.8, 55.4, 53.5, 52.5, 44.0, 36.2, 29.6, 22.8.

### EXAMPLE 563

### Synthesis of N-(4-Methoxybenzyl)-L-pyroglutamyl-L-4-(phenylcarbonylamino)phenylalanine

The title compound was prepared from the product of Example 562 using the procedure described in Method F.

NMR data was as follows:

¹H NMR (DMSO-d₆): δ 10.25 (s, 1H), 8.59 (d, 1H), 7.94 (d, 2H), 7.74 (d, 2H), 7.55 (m, 3H), 7.42 (d, 2H), 6.92 (d, 2H), 6.83 (d, 2H), 4.73 (d, 1H), 4.60 (m, 1H), 3.83 (m, 1H), 3.70 (s, 3H), 3.32 (d, 1H), 3.16-2.81 (m, 2H), 2.30 (m, 2H), 2.10 (m, 1H), 1.73 (m, 1H).

### EXAMPLE 564

### Synthesis of N-(3-Chlorobenzyl)-L-pyroglutamyl-L-(N′-benzyl)histidine

N-(3-Chlorobenzyl)-L-pyroglutamyl-L-(N′-benzyl)histidine methyl ester was prepared from the appropriate starting materials using the procedure described in Method B. The title compound was then prepared by hydrolysis of the methyl ester using the procedure described in Method E.

NMR data was as follows:

¹H NMR (DMSO-d₆): δ 8.08 (brd, 1H), 7.59 (s, 1H), 7.38-7.12 (m, 8H), 7.05 (s, 1H), 6.84 (s, 1H), 5.08 (m, 2H), 4.68 (d, 1H), 4.27 (m, 1H), 3.97 (m, 1H), 3.59 (d, 1H), 3.05-2.70 (m, 2H), 2.26 (m, 2h), 2.08 (m, 1H), 1.79 (m, 1H).

¹³C NMR (DMSO-d₆): δ 175.3, 173.9, 170.5, 139.9, 139.3, 138.1, 136.9, 133.5, 130.7, 129.0, 128.0, 127.8, 127.6, 127.0, 116.7, 59.8, 54.0, 49.8, 44.1, 31.1, 29.6, 22.8.

### EXAMPLE 565

### Synthesis of N-(4-Methylbenzyl)-L-pyroglutamyl-L-(N′-benzyl)histidine Methyl Ester

The title compound was prepared from the appropriate starting materials using the procedures described in Examples 552 and 564.

NMR data was as follows:

¹H NMR (CDCl₃): δ 8.18 (d, 1H), 7.42 (s, 1H), 7.33 (m, 3H), 7.16-7.06 (m, 6H), 6.67 (s, 1H), 5.14 (d, 1H), 5.04 (s, 2H), 4.79 (m, 1H), 3.88 (m, 1H), 3.82 (d, 1H), 3.64 (s, 3H), 3.15-2.94 (m, 2H), 2.70-2.57 (m, 1H), 2.39 (m, 1H), 2.27-2.00 (m, 2H).

¹³C NMR (CDCl₃): δ 176.2, 172.1, 172.1, 138.3, 138.0, 137.9, 136.4, 133.5, 130.0, 129.6, 129.2, 120.0, 127.8, 117.5, 60.8, 53.9, 53.9, 51.5, 45.5, 30.9, 29.5, 23.9, 21.7.

### EXAMPLE 566

### Synthesis of N-(4-Methylbenzyl)-L-pyroglutamyl-L-(N′-benzyl)histidine

The title compound was prepared from the product of Example 565 using the procedures described in Method E.

NMR data was as follows:

¹H NMR (D₂O): δ 7.70 (s, 1H), 7.35-7.20 (m, 5H), 7.08 (d, 2H), 6.98 (s, 1H), 6.74 (d, 2H), 5.05 (s, 2H), 4.49-4.42 (m, 2H), 3.94 (m, 1H), 3.31 (d, 1H), 3.14-2.73 (m, 2H), 2.24 (s, 3H), 2.56-2.11 (m, 3H), 1.91 (m, 1H).

### EXAMPLE 567

### Synthesis of N-Benzyl-D-pyroglutamyl-L-phenylalanine

The title compound was prepared from the appropriate starting materials using the procedures described in Examples 552 and 553.

NMR data was as follows:

¹H NMR (DMSO-d₆): δ 8.52 (d, 1H), 7.4-7.1 (m, 10H), 6.97 (d, 1H), 4.83 (dd, 2H), 4.73 (dd), 4.50 (m, 1H), 3.84 (m, 1H), 3.50 (dd, 2H), 3.40 (dd), 3.13 (2H), 2.85 (2H), 2.19 (m, 2H), 2.03 (m, 1H), 1.48 (m, 1H).

¹³C NMR (DMSO-d₆): δ 175.0, 173.2, 171.3, 138.0, 136.9, 129.5, 129.4, 128.9, 128.9, 128.6, 128.4, 128.2, 127.7, 126.8, 57.1, 53.5, 44.5, 35.8, 29.5, 22.9.

### EXAMPLE 568

### Synthesis of N-(4-Benzyl-3-oxothiomorpholin-5-carbonyl)-L-phenylalanine

The title compound was prepared from the product of Example 569 using the procedure described in Method F.

NMR data was as follows:

¹H NMR (CDCl₃): δ 7.38-6.98 (m, 10H), 5.48 (d, 1H), 4.97 (m, 1H), 4.20 (t, 1H), 4.09 (t), 3.67 (d, 1H), 3.50-2.78 (m, 6H).

¹³C NMR (CDCl₃): δ 175.5, 169.7, 168.0, 136.2, 135.8, 129.9, 129.5, 129.3, 129.0, 128.7, 128.0, 62.5, 53.9, 51.1, 38.0, 31.3, 29.4.

### EXAMPLE 569

### Synthesis of N-(4-Benzyl-3-oxothiomorpholin-5-carbonyl)-L-phenylalanine Ethyl Ester

### Step A—Preparation of N-Benzyl-3-oxothiomorpholin-5-carboxylic Acid

S-(Methylcarboxyethyl)cysteine (Biochemistry, 1989, 28(2), 465) (1.633 g, 7.88 mmol) was placed in MeOH (50 mL) and benzaldehyde (0.8 mL, 7.88 mmol) was added. The mixture was stirred for 10 minutes and then sodium cyanoborohydride (0.594 g, 946 mmol) was added. The reaction was stirred overnight under N₂ and then filtered to afford 853 mg of a white solid. This white solid was then heated in water overnight to afford N-benzyl-3-oxothiomorpholin-5-carboxylic acid as a white solid.

### Step B—Preparation of N-(4-Benzyl-3-oxothiomorpholin-5-carbonyl)-L-phenylalanine Ethyl Ester

The title compound was prepared from the product of Step A and L-phenylalanine ethyl ester using the procedures described in Method B.

NMR data was as follows:

¹H NMR (CDCl₃): δ 7.38-7.15 (m, 10H), 6.57 (d, 1H), 5.54 (d, 1H), 4.17 (m, 1H), 4.89 (q, 1H), 4.20 (q, 2H), 3.09 (d, 1H) 3.46 (d, 1H), 3.25-2.80 (m, 5H), 1.29 (t, 3H).

¹³C NMR (CDCl₃): δ 171.6, 169.4, 168.6, 136.3, 136.1, 129.8, 129.7, 129.5, 129.3, 129.0, 128.6, 127.0, 62.4, 62.4, 53.8, 50.7, 38.1, 31.3, 29.4, 14.8.

### EXAMPLE 570

### Synthesis of N-(4-Benzyl-3-oxothiomorpholin-5-carbonyl)-L-4-nitrophenylalanine Methyl Ester

The title compound was prepared from the appropriate starting materials using the procedures described in Example 569.

NMR data was as follows:

¹H NMR (CDCl₃): δ 8.16 (d, 2H), 7.42-7.17 (m, 7H), 6.84 (d, 1H), 5.63 (d, 1H), 4.99 (m, 1H), 4.18 (m, 1H), 3.78-3.70 (m, 4H), 3.56 (d, 1H), 3.38-3.15 (m, 3H), 3.05-2.87 (m, 2H).

¹³C NMR (CDCl₃): δ 171.5, 169.7, 166.5, 147.8, 144.1, 136.1, 130.8, 129.5, 128.9, 128.8, 124.4, 62.5, 53.7, 53.5, 50.9, 38.1, 31.5, 29.6.

### EXAMPLE 571

### Synthesis of N-Benzyl-L-pyroglutamyl-L-4-(N,N-dimethylcarbamyloxy)phenylalanine Isopropyl Ester

The title compound was prepared from the appropriate starting materials using the procedure described in Method L to afford white crystals, mp 167-169° C.

Physical data was as follows:

Anal. Calcd. for C₂₇H₃₃N₃O₆: C, 65.44; H, 6.71; N, 8.48. Found: C, 65.06; H, 6.73; N, 8.42.

MS (+EI): 495 (M+)+.

### EXAMPLE 572

### Synthesis of N-Benzyl-L-pyroglutamyl-L-3-chloro-4-(N,N-dimethylcarbamyloxy)phenylalanine Methyl Ester

N-Benzyl-L-pyroglutamyl-L-3-chloro-4-hydroxyphenylalanine methyl ester was prepared from the appropriate starting materials using the procedure described in Method B. The title compound was then prepared from the methyl ester using the procedure described in Method L to afford a white solid.

Physical data was as follows:

Anal. Calcd. for C₂₅H₂₈ClN₃O₆-0.1 CH₂Cl₂: C, 59.06; H, 5.57; N, 8.33. Found: C, 59.08; H, 5.37; N, 8.24.

MS (+ESI): 502 (M+1)+.

### EXAMPLE 573

### Synthesis of N-(4-Fluorobenzyl)-L-pyroglutamyl-L-4-(N,N-dimethylcarbamyloxy)phenylalanine

The title compound was prepared from the appropriate starting materials using the sequential application of the procedure described in Methods B, L and N to afford a white solid, mp 227-230° C.

Physical data was as follows:

Anal. Calcd. for C₂₄H₂₆FN₃O₆: C, 61.14; H, 5.56; N, 8.91. Found: C, 60.80; H, 5.48; N, 8.81.

MS (+ESI): 472 (M+1)+.

### EXAMPLE 574

### Synthesis of N-(4-Fluorobenzyl)-L-pyroglutamyl-L-4-[(thiomorpholin-4′-yl)carbonyloxy)phenylalanine

N-(4-Fluorobenzyl)-L-pyroglutamyl-L-4-[(thiomorpholin-4′-yl)carbonyloxy)phenylalanine tert-butyl ester was prepared from the appropriate starting materials using the procedure described in Method M (thiomorpholine was substituted for N-methylpiperazine). The title compound was then prepared from the tert-butyl ester using the procedure described in Method N to afford a white solid, mp 266-268° C. (dec.) Physical data was as follows:

Anal. Calcd. for C₂₆H₂₈FN₃O₆S: C, 58.97; H, 5.33; N, 7.93. Found: C, 57.98; H, 5.09; N, 7.62.

MS (−ESI): 528 (M−1)−.

### EXAMPLE 575

### Synthesis of N-(4—Cyanobenzyl)-L-pyroglutamyl-L-4-(N,N-dimethylcarbamyloxy)phenylalanine

The title compound was prepared from the appropriate starting materials using the sequential application of the procedures described in Methods B, L and N to afford a white solid, mp 232-236° C. (dec).

Physical data was as follows:

Anal. Calcd. for C₂₅H₂₆N₄O₆-0.5H₂O.0.08C₄H₁₀O C, 61.63; H, 5.68; N, 11.35. Found: C, 62.01; H, 5.51; N, 11.00.

MS (+APCI): 479 (M+1)+.

### EXAMPLE 576

### Synthesis of N-(4-Nitrobenzyl)-L-pyroglutamyl-L-4-(N,N-dimethylcarbamyloxy)phenylalanine tert-Butyl Ester

The title compound was prepared from the appropriate starting materials using the sequential application of the procedures described in Methods B and L to afford white crystals, mp 159-161° C.

Physical data was as follows:

Anal. Calcd. for C₂₈H₃₄N₄O₈: C, 60.64; H, 6.18; N, 10.10. Found: C, 60.41; H, 6.34; N, 9.73.

MS (+ESI): 555 (M+1)+.

### EXAMPLE 577

### Synthesis of N-Benzyl-L-pyroglutamyl-L-3-chloro-4-(N,N-dimethylcarbamyloxy)phenylalanine

The title compound was prepared from the product of Example 572 using the procedure described in Method D to afford a white solid.

Physical data was as follows:

Anal. Calcd. for C₂₄H₂₅ClN₃O₆Li.2.5H₂O: C, 53.49; H, 5.61; N, 7.80. Found: C, 53.18; H, 5.02; N, 7.59.

MS (+ESI): 488 (M+1)+.

### EXAMPLE 578

### Synthesis of N-(4-Fluorobenzyl)-L-pyroglutamyl-L-4-[(4′-(pyridin-2′-yl)piperazin-1′-yl)carbonyloxy]phenylalanine

The title compound was prepared from the product of Example 579 using the procedure described in Method N.

Physical data was as follows:

Anal. Calcd. for C₃₁H₃₂FN₅O₆.2.5 HCO₂H: C, 60.47; H, 5.39; N, 11.02. Found: C, 57.31; H, 5.69; N, 9.53.

MS (−ESI): 588 (M−1)−.

### EXAMPLE 579

### Synthesis of N-(4-Fluorobenzyl)-L-pyroglutamyl-L-4-[(4′-(pyridin-2′-yl)piperazin-1′-yl)carbonyloxy]phenylalanine tert-Butyl Ester

The title compound was prepared from the appropriate starting materials using the procedures of Method M (1-(pyridin-2-yl)piperazine was substituted for N-methylpiperazine) to afford white crystals, mp 198-199° C.

Physical data was as follows:

Anal. Calcd. for C₃₅H₄₀FN₅O₆: C, 65.10; H, 6.24; N, 10.85. Found: C, 65.04; H, 6.17; N, 10.77.

MS (+ESI): 646 (M+1)+.

### EXAMPLE 580

### Synthesis of N-(Pyridin-3-ylmethyl)-L-pyroglutamyl-L-tyrosine tert-Butyl Ester

### Step A—Preparation of N-(Pyridin-3-ylmethyl)-L-pyroglutamic Acid Methyl Ester

N-(Pyridin-3-ylmethyl)-L-pyroglutamic acid methyl ester was prepared by reductive alkylation of L-glutamic acid with the appropriate aldehyde followed by acid catalyzed cyclization employing the procedures described in J. Amer. Chem. Soc. 106, 4539 (1984). The following work-up procedures were employed: after the aqueous solution (pH=3) was heated overnight, the solution was cooled to 25° C. and the pH was adjusted to 7 using 2N NaOH. The aqueous phase was lyophilized to a gummy solid which was treated with methanolic HCl overnight. After filtration, the solvent was evaporated to afford the crude methyl ester which was taken up in CH₂Cl₂ and washed with saturated sodium bicarbonate, followed by saturated brine, and then dried over MgSO₄ and evaporated to an oil. This oil was then flash chromatographed on alumina (activity grade 3) using ethyl acetate/hexane 1:1 as the eluent to afford N-(pyridin-3-ylmethyl)-L-pyroglutamic acid methyl ester as a colorless oil.

### Step B—Preparation of N-(Pyridin-3-ylmethyl)-L-pyroglutamyl-L-tyrosine tert-Butyl Ester

The title compound was prepared by reacting the acid obtained from the hydrolysis of the product of Step A (using Method D) and L-tyrosine tert-butyl ester following the procedures described in Method B.

Physical data was as follows:

Anal. Calcd. for C₂₄H₂₉N₃O₅.0.22C₃H₇NO.0.7H₂O: C, 63.26; H, 6.87; N, 9.63. Found: C, 63.16; H, 6.60; N, 9.44.

MS (+ESI): 440 (M+1)+.

### EXAMPLE 581

### Synthesis of N-(Pyridin-3-ylmethyl)-L-pyroglutamyl-L-4-(N,N-dimethylcarbamyloxy)phenylalanine

The title compound was prepared from the product of Example 582 using the procedure described in Method N to afford a white solid.

Physical data was as follows:

Anal. Calcd. for C₂₃H₂₆N₄O₆. 0.12C₄H₈O₂.0.25H₂O: C, 60.05; H, 5.89; N, 11.93. Found: C, 59.94; H, 5.77; N, 11.91.

MS (+ESI): 455 (M+1)+.

### EXAMPLE 582

### Synthesis of N-(Pyridin-3-ylmethyl)-L-pyroglutamyl-L-4-(N,N-dimethylcarbamoyloxy)phenylalanine tert-Butyl Ester

The title compound was prepared from the product of Example 580 using the procedure described in Method M to afford a crystalline solid, mp 157-158° C.

Physical data was as follows:

Anal. Calcd. for C₂₇H₃₄N₄O₆: C, 63.51; H, 6.71; N, 10.97. Found: C, 63.35; H, 6.75; N, 10.88.

MS (+ESI): 511 (M+1)+.

### EXAMPLE 583

### Synthesis of N-(Pyridin-3-ylmethyl)-L-pyroglutamyl-L-4-[(4′-(pyridin-2′-yl)piperazin-1′-yl)carbonyloxy]phenylalanine tert-Butyl Ester

The title compound was prepared from the appropriate starting materials using the procedures of Method M (1-(pyridin-2-yl)piperazine was substituted for N-methylpiperazine) to afford a white solid.

Physical data was as follows:

Anal. Calcd. for C₃₄H₄₀N₆O₆: C, 64.95; H, 6.41; N, 13.37. Found: C, 64.94; H, 6.40; N, 13.18.

MS (+ESI): 629 (M+1)+.

### EXAMPLE 584

### Synthesis of N-(Pyridin-3-ylmethyl)-L-pyroglutamyl-L-4-[(4′-(pyridin-2′-yl)piperazin-1′-yl)carbonyloxy]phenylalanine

The title compound was prepared from the product of Example 583 using the procedure described in Method N to afford a white solid.

Physical data was as follows:

Anal. Calcd. for C₃₀H₃₂N₆O₆: C, 62.93; H, 5.63; N, 14.68. Found: C, 62.20; H, 5.49; N, 14.22.

MS (−ESI): 571 (M−1)−.

### EXAMPLE 585

### Synthesis of N-(4-Benzyl-5-oxo-4-azatricyclo[4.2.1.0 (3,7)]nonane-3-carbonyl)-L-tyrosine tert-Butyl Ester

### Step A—Preparation of 5-Oxo-4-azatricyclo[4.2.1.0 (3,7)]nonane-3-carboxylic acid

A solution of endo-6-carboxybicyclo[2.2.1]heptane-2-one (6.72 g, 43.6 mmol (J. Org. Chem. 41:1233 (1976)), KCN (3.41 g, 52.4 mmol) and (NH₄)₂CO₃ (16.77 g, 174.6 mmol) in 206 mL 1:1H₂O-ethanol was heated 24 h at 55° C. The condenser was then removed and the reaction mixture was refluxed for 1.5 h. After the reaction was acidified with conc. HCl and cooled to 5° C., a precipitate was obtained which after washing with H₂O and dried to afford 1.74 g (18%) of a white solid, mp 286° C. This intermediate (1.74 g, 7.76 mmol) was converted to the title compound by refluxing in 30 mL of 2.5 N NaOH for 24 h. Acidification to pH=0 gave the desired product as a white solid, mp 298-300° C.

Physical data was as follows:

Anal. Calcd. for C₉H₁₁NO₃: C, 59.66; H, 6.12; N, 7.73. Found: C, 59.39; H, 6.24; N, 7.67.

### Step B—Preparation of 5-Oxo-4-azatricyclo[4.2.1.0 (3.7)]nonane-3-carboxylic Acid Methyl Ester

To a suspension of 5-oxo-4-azatricyclo[4.2.1.0 (3,7)]nonane-3-carboxylic acid (183 mg, 1.0 mmol) in 10 mL MeOH under nitrogen at −78° C. was added SOCl₂ (75 μL, 1.0 mmol). After 169 h, the solvent was evaporated to afford 195 mg of a white solid which was taken up in 10 mL CHCl₃, washed sequentially with 10 mL saturated NaHCO₃ and 10 mL saturated NaCl, dried over MgSO₄ and evaporated to give 160 mg (81%) of a white solid, mp 142-144° C.

Physical data was as follows:

MS (FI-POS): 196 (M+1)+.

### Step C—Preparation of 4-Benzyl-5-oxo-4-azatricyclo[4.2.1.0 (3.7)]nonane-3-carboxylic Acid Methyl Ester

To a suspension of 5-oxo-4-azatricyclo[4.2.1.0 (3,7)]nonane-3-carboxylic acid methyl ester (68 8 mg, 3.52 mmol) in 10 mL of THF under nitrogen at 25° C. was added LiHMDS (3.87 mL of 1N THF solution, 3.87 mmol). After 15 min, benzyl bromide (0.42 mL, 3.53 mmol) was added. After 169 h, the reaction was quenched by addition of 10 mL of saturated NH₄Cl solution. The reaction mixture was partitioned between 30 mL CH₂Cl₂ and 10 mL H₂O. The organic phase was separated, washed with 50 mL saturated NaCl, dried over MgSO₄ and evaporated to give 770 mg of an oil. Flash chromatography of 740 mg of this material, eluting with 95:5 CH₂Cl₂-EtOAc, afforded 510 mg (51%) of the title compound as a colorless oil (0.14 CH₂Cl₂ solvate).

Physical data was as follows:

MS (+ESI): 286 (M+1)+.

### Step D—Preparation of 4-Benzyl-5-oxo-4-azatricyclo[4.2.1.0 (3.7)]nonane-3-carboxylic Acid

To a solution of 4-benzyl-5-oxo-4-azatricyclo[4.2.1.0 (3,7)]nonane-3-carboxylic acid methyl ester.0.14 methylene chloride solvate (399 mg, 1.34 mmol) in 13 mL MeOH under nitrogen at 25° C. was added 1.5 mL of 1N LiOH. After 117 h, most of the solvent was removed and the residue was taken up in 10 mL 1N NaOH, washed with 2×10 mL Et₂O, acidified by addition of 10 mL 2 N HCl, extracted 2× with 10 mL Et₂O, dried over MgSO₄ and evaporated to give 225 mg (71%) of a white solid, mp 191-194° C.

Physical data was as follows:

Anal. Calcd. for C₁₆H₁₇NO₃: C, 70.83; H, 6.32; N, 5.16. Found: C, 70.56; H, 6.39; N, 5.01.

### Step E—Preparation of N-(4-Benzyl-5-oxo-4-azatricyclo[4.2.1.0 (3,7)]nonane-3-carbonyl)-L-tyrosine tert-Butyl Ester

The title compound was prepared from 4-benzyl-5-oxo-4-azatricyclo[4.2.1.0 (3,7)]nonane-3-carboxylic acid (0.60 mmol) and L-tyosine tert-butyl ester using the procedures described in Example 552 to afford 300 mg (93%) of a white solid.

Physical data was as follows:

Anal. Calcd. for C₂₉H₃₄N₂O₅.0.5C₄H₈O₂: C, 69.64; H, 7.16; N, 5.24. Found: C, 69.41; H, 7.02; N, 5.34.

MS (+ESI): 491 (M+1)+.

### EXAMPLE 586

### Synthesis of N-(4-Benzyl-5-oxo-4-azatricyclo[4.2.1.0 (3,7)]nonane-3-carbonyl)-L-4-(N,N-dimethylcarbamyloxy)phenylalanine tert-Butyl Ester

The title compound was prepared from the product of Example 585 using the procedure described in Method L to afford a white solid.

Physical data was as follows:

Anal. Calcd. for C₃₂H₃₉N₃O₆: C, 68.43; H, 7.00; N, 7.48. Found: C, 67.98; H, 7.00; N, 7.27.

MS (+ESI): 562 (M+1)+.

### EXAMPLE 587

### Synthesis of N-(4-Benzyl-5-oxo-4-azatricyclo[4.2.1.0 (3,7)]nonane-3-carbonyl)-L-4-(N,N-dimethylcarbamyloxy)phenylalanine

The title compound was prepared from the product of Example 586 using the procedure described in Method N to afford a white solid.

Physical data was as follows:

Anal. Calcd. for C₂₈H₃₁N₃O₆.0.5C₄H₁₀₀: C, 66.40; H, 6.69; N, 7.74. Found: C, 65.72; H, 6.42; N, 7.95.

MS (+ESI): 506 (M+1)+.

### EXAMPLE 588

### Synthesis of N-(Benzyl)-L-pyroglutamyl-L-tyrosine Ethyl Ester

To a solution of N-benzyl-L-pyroglutamic acid (J. Am. Chem. Soc. 106:4539 (1984), 1.00 g, 4.56 mmol), L-tyrosine ethyl ester hydrochloride (1.23 g, 5.01 mmol) and BOP (2.22 g, 5.01 mmol) in DMF (32 mL) under nitrogen was added triethylamine (14 g, 11.26 mmol) dropwise and the resulting solution was stirred at ambient temperature for 22.5 h. The reaction was quenched by addition of 150 mL of saturated sodium bicarbonate and 150 mL of EtOAc. The organic layer was separated and washed sequentially with 150 mL H₂O, 150 mL 10% citric acid and 150 mL saturated brine, dried over MgSO₄ and evaporated to 1.6 g (82%) of a white solid, mp 192-194° C.

Physical data was as follows:

¹H NMR (DMSO-d₆, 400 MHz) δ 9.23 (s, 1H); 8.52 (d, 1H, J=7.9 Hz); 7.32-7.23 (m, 3H); 7.03-6.97 (m, 4H); 6.69-6.65 (m, 2H); 4.75 (d, 1H, J=15.2 Hz); 4.49-4.43 (m, 1H); 4.11-4.01 (m, 2H); 3.88-3.85 (m, 1H); 3.37 (d, 1H, J=15.2 Hz); 2.99-2.94 (m, 1H); 2.78-2.72 (m, 1H); 2.33-2.08 (m, 3H); 1.98 (s, 0.2H); 1.76-1.70 (m, 1H); 1.14 (t, 3H, J=7.25 Hz).

IR (KBr, cm⁻¹) 3400; 3250; 3060; 1725; 1680; 1670; 1550; 1510; 1440; 1265; 1220.

MS (−FAB) 409.1 (M−H); 381.0; 302.0; 275.0; 257.0; 217.0; 183.0; 91.0.

Anal. Calcd. for C₂₃H₂₆N₂O₅.0.2M EtOAc: C, 66.78; H, 6.50; N, 6.54. Found: C, 66.48; H, 6.35; N, 6.66.

### EXAMPLE 589

### Synthesis of N-(Benzyl)-L-pyroglutamyl-L-tyrosine

To a solution of N-(benzyl)-L-pyroglutamyl-L-tyrosine ethyl ester 0.2 ethyl acetate solvate (0.139 g, 0.325 mmol) in MeOH (3.25 mL) under nitrogen was added 1 N aqueous LiOH (0.72 mL, 0.72 mmol). After 3 days, the solvent was evaporated and the residue was partitioned between 10 mL H₂O and 10 mL CH₂Cl₂. The aqueous layer was washed with 10 mL CH₂Cl₂ and acidified to pH=1 by addition of 7 mL 1 N HCl. The precipitate was filtered, washed with 20 mL 1:1 CHCl₃/EtOAc and dried to afford 0.0835 g of a white solid (67%).

Physical data was as follows:

¹H NMR (DMSO-d₆, 400 MHz) δ 12.76 (brd s, 1H); 9.22 (s, 1H); 8.40 (d, 1H, J=8.35 Hz); 7.32-7.23 (m, 3H); 7.02-6.97 (m, 4H); 6.68-6.65 (m, 2H); 4.74 (d, 1H, J=14.94 Hz); 4.47-4.41 (m, 1H); 3.87-3.84 (m, 1H); 3.40-3.24 (m, 1H); 3.03-2.98 (m, 1H); 2.75-2.69 (m, 1H); 2.35-2.04 (m, 3H); 1.76-1.69 (m, 1H).

IR (KBr, cm⁻¹) 3280; 1745; 1670; 1660; 1550; 1515; 1255; 1200; 820; 700.

MS (+FAB) 383.1 (M+H); 367.1; 327.0; 311.0; 295.0; 279.0; 237.0; 197.0; 174.1; 136.0; 105.0.

Anal. Calcd. for C₂₁H₂₂N₂O₅.0.2M−H₂O: C, 65.35; H, 5.85; N, 7.26. Found: C, 65.14; H, 5.66; N, 7.13.

### EXAMPLE 590

### Synthesis of N-(Benzyl)-L-pyroglutamyl-L-4-[(4′-methylpiperazin-1′-yl)carbonyloxy]phenylalanine Ethyl Ester

To a suspension of N-(benzyl-L-pyroglutamyl)-L-tyrosine ethyl ester 0.2 ethyl acetate solvate (0.894 g, 2.09 mmol) and 4-nitrophenyl chloroformate (0.435 g, 2.09 mmol) in 13 mL of 1:1 CH₃CN/CH₂Cl₂ under nitrogen at 0° C. was added DMAP (0.032 g, 0.26 mmol) followed by TEA (0.529 g, 5.22 mmol). After 30 min at 0° C. the reaction mixture was warmed up to 25° C. and kept at this temperature for 30 min. The reaction was then cooled back down to 0° C. and 1-methylpiperazine (0.206 g, 1.06 mmol) was added. The ice bath was then removed and the reaction mixture was stirred at 25° C. for 3 h 15 min. The reaction mixture was then diluted with 50 mL Et₂O and washed with 4×25 mL 10% sodium carbonate, diluted with CH₂Cl₂, dried over K₂CO₃ and evaporated to 1.0 g (88%) of crude solid which was recrystallized from toluene/hexane to afford white crystals, mp 145-148° C.

Physical data was as follows:

¹H NMR (DMSO-d₆, 400 MHz) δ 8.59 (d, 1H, J=8.1 Hz); 7.31-7.19 (m, 5H); 7.04-7.00 (m, 4H); 4.76 (d, 1H, J=15.2 Hz); 4.56-4.51 (m, 1H); 4.11-4.05 (m, 2H); 3.87-3.84 (m, 1H); 3.54-3.35 (m, 5H); 3.10-3.05 (m, 1H); 2.90-2.84 (m, 1H); 2.32-2.09 (m, 10H); 1.74-1.69 (m, 1H); 1.24-1.23 (m, 0.4H); 1.15 (t, 3H, J=7.0 Hz); 0.85 (m, 0.3H).

IR (KBr, cm⁻¹) 3300; 1740; 1720; 1680; 1650; 1550; 1410; 1240; 1210; 1200; 1160; 700.

MS (EI) 536 (M+); 491; 234; 174; 127; 91; 83; 70; 58; 44.

Anal. Calcd. for C₂₉H₃₆N₄O₆.0.1M C₆H₁₄: C, 65.22; H, 6.90; N, 10.28. Found: C, 65.06; H, 6.66; N, 9.88.

### EXAMPLE 591

### Synthesis of N-(Benzyl)-L-pyroglutamyl-L-4-(1′-methylpiperidin-4′-yloxy)phenylalanine Lithium Salt

To a solution of N-(benzyl)-L-pyroglutamyl-L-4-[(4′-methylpiperazin-1′-yl)carbonyloxy]phenylalanine ethyl ester 0.1 hexane solvate (0.200 g, 0.367 mmol) in MeOH (3.67 mL) under nitrogen was added 1 N aqueous LiOH (0.35 mL, 0.35 mmol). After 28 h the solvent was removed, 25 mL H₂O was added, the aqueous solution was washed twice with 25 mL of CH₂Cl₂, filtered and lyophilized to afford 0.16 g (77%) of a white solid.

Physical data was as follows:

¹H NMR (DMSO-d₆, 400 MHz) δ 7.60 (d, 2H, J=7.3 Hz); 7.30-7.21 (m, 3H); 7.13-7.10 (m, 2H); 7.05-7.03 (m, 2H); 6.92-6.88 (m, 2H); 4.75 (d, 1H, J=15.2 Hz); 4.04-4.01 (m, 1H); 3.89-3.86 (m, 1H); 3.54-3.33 (m, 1H); 3.13-3.09 (m, 1H); 2.92-2.87 (m, 1H); 2.33-2.05 (m, 10H); 1.79-1.75 (m, 1H).

IR (KBr, cm⁻¹) 3400; 1720; 1680; 1610; 1420; 1240; 1200; 1160; 1000; 710.

MS (+FAB) 515.0 (M+Li); 471.0; 220.9; 174.0; 91.0.

Anal. Calcd. for C₂₇H₃₁N₄O₆Li.3.0M H₂O: C, 57.04; H, 6.56; N, 9.85. Found: C, 57.27; H, 5.70; N, 9.59.

### EXAMPLE 592

### Synthesis of N-(Benzyl)-L-pyroglutamyl-L-4-(pyridin-4-ylcarbonylamino)phenylalanine Methyl Ester

A solution of N-benzyl-L-pyroglutamic acid (J. Am. Chem. Soc. 106:4539 (1984), 1.00 g, 4.562 mmol), 4-[(4-pyridinylcarbonyl)amino]-L-phenylalanine methyl ester (1.532 g, 4.562 mmol) and BOP (2.018 g, 4.562 mmol) in acetonitrile (30.0 mL) was charged to a 100 mL round bottom flask equipped with a stir bar and nitrogen inlet. Triethylamine (1.272 mL, 9.123 mmol) was added dropwise and the resulting solution was stirred at 25° C. for 16 h. The solvent was stripped off and the material taken up in methylene chloride (75 mL) and washed with saturated sodium bicarbonate solution (50 mL×3), dried (K₂CO₃) and the solvent removed to give a biege solid (2.000 g). This material was chromatographed on silica gel (9:1 CH₂Cl₂:CH₃OH) yielding a white solid which was recrystallized from acetonitrile to provide 1.744 g (76%) of white needles, mp=204-208° C.

Physical data was as follows:

¹H NMR (DMSO-d₆, 400 MHz) δ 10.47 (s, 1H); 8.77 (d, 2H, J=6.2 Hz); 8.60 (d, 1H, J=7.9 Hz); 7.83 (d, 2H, J=6.2 Hz); 7.71 (d, 2H, J=8.6 Hz); 7.31-7.19 (m, 5H); 7.02 (d, 2H, J=5.1 Hz); 4.76 (d, 1H, J=14.9 Hz); 4.61-4.55 (m, 1H); 3.87-3.84 (m, 1H); 3.65 (s, 3H); 3.36 (d, 1H, J=14.4 Hz); 3.11-3.06 (m, 1H); 2.89-2.83 (m, 1H); 2.34-2.21 (m, 2H); 2.17-2.09 (m, 1H); 1.76-1.70 (m, 1H).

IR (KBr, cm⁻¹) 3400; 3325; 3100; 3030; 2960; 1660; 1625; 1540; 1490; 1425; 1325; 1225; 700.

MS (+FAB) 501.1 (M+H); 485.1; 475.1; 465.1; 451.0; 394.1; 279.0; 174.0; 91.0.

Anal. Calcd. for C₂₈H₂₈N₄O₅. 0.15 CH₂Cl₂: C, 65.87; H, 5.56; N, 10.92. Found: C, 65.87; H, 5.70; N, 11.36.

### EXAMPLE 593

### Synthesis of N-(Benzyl)-L-pyroglutamyl-L-4-(pyridin-4-ylcarbonylamino)phenylalanine Lithium Salt

N-(Benzyl)-L-pyroglutamyl-L-4-(pyridin-4-ylcarbonylamino)-phenylalanine methyl ester 0.15 methylene chloride solvate (0.400 g, 0.799 mmol) was taken up in methanol (15 mL) and the solvent stripped off using a rotovap. This procedure was repeated twice more to remove any traces of acetonitrile. The solid was dissolved in methanol (15 mL) and charged to a 25 mL round bottom flask equipped with a magnetic stir bar, air condenser and nitrogen inlet. The mixture was warmed until the ester dissolved (oil bath temperature=40° C.) and 1N LiOH (759 μL, 0.759 mmol) was added, via syringe, and the solution stirred for 16 h under nitrogen. The reaction solution was transferred to a 50 mL round bottom flask and the solvent stripped off yielding a white solid (0.731 g). This solid was taken up in water (50 mL) and washed with methylene chloride (25 mL). An emulsion formed and was allowed to separate. The organic phase was separated and the aqueous phase was filtered, pumped on for 3 h and lyophilized to give 0.353 g (94%) of a fluffy white solid, mp=347° C. (decompose).

Physical data was as follows:

¹H NMR (DMSO-d₆, 400 MHz) δ 10.48 (s, 1H); 8.76-8.74 (m, 2H); 7.85-7.83 (m, 2H); 7.60 (t, 2H, J=8.6 Hz); 7.53 (d, 1H, J=6.8 Hz); 7.30-7.20 (m, 3H); 7.15-7.09 (m, 2H); 7.04 (d, 2H, J=7.6 Hz); 4.77 (d, 1H, J=15.4 Hz); 4.01-3.98 (m, 1H); 3.90-3.86 (m, 1H); 3.43 (d, 1H, J=15.2 Hz); 3.13-3.08 (m, 1H); 2.94-2.89 (m, 1H); 2.29-2.19 (m, 2H); 2.11-2.05 (m, 1H); 1.80-1.77 (m, 1H).

IR (KBr, cm⁻¹) 3325; 3030; 2960; 1660; 1600; 1530; 1425; 1325; 830; 700.

MS (+FAB) 487.0 (M+H); 471.0; 450.9; 429.0; 417.0; 400.9; 385.0; 279.0; 236.9; 91.0.

Anal. Calcd. for C₂₇H₂₆N₄O₅.3.00H₂O C, 58.34; H, 5.72; N, 10.25. Found: C, 58.45; H, 5.44; N, 9.95.

### EXAMPLE 594

### Synthesis of N-(Benzyl)-L-pyroglutamyl-L-4-nitrophenylalanine Ethyl Ester

A solution of N-benzyl-L-pyroglutamic acid (J. Am. Chem. Soc. 106:4539 (1984), 1.00 g, 4.562 mmol), (S)-4-nitro-phenylalanine ethyl ester (1.262 g, 4.562 mmol) and HOBT (1.233 g, 9.123 mmol) in methylene chloride (40.0 mL) was charged to a 100 mL round bottom flask equipped with a stir bar and nitrogen inlet. Hunig's base (3.25 mL, 18.246 mmol) was added dropwise. The solution remained heterogeneous so acetonitrile (10 mL) was added followed by the addition of EDC (1.749 g, 9.123 mmol) and the resulting milk white mixture was stirred at 25° C. for 16 h. The solvent was stripped off and the solid taken up in ethyl acetate (100 mL) and washed with saturated ammonium chloride solution (100 mL×2), saturated sodium bicarbonate solution (50 mL×2), brine (50 mL×2), dried (MgSO₄) and the solvent removed to yield 1.166 g (58%) of a yellow solid. This material was recrystallized from ethyl acetate giving a white solid, mp=186-187° C.

Physical data was as follows:

¹H NMR (DMSO-d₆, 400 MHz) δ 8.63 (d, 1H, J=8.1 Hz); 8.15 (d, 2H, J=9.1 Hz); 7.51 (d, 2H, J=8.8 Hz); 7.29-7.23 (m, 3H); 6.99-6.95 (m, 2H); 4.76 (d, 1H, J=15.2 Hz); 4.69-4.64 (m, 1H); 4.11 (q, 2H, J=5.5 Hz); 3.82-3.79 (m, 2H); 3.37 (d, 1H, J=15.2 Hz); 3.28-3.23 (m, 1H); 3.06-3.00 (m, 1H); 2.33-2.21 (m, 2H); 2.17-2.09 (m, 1H); 1.73-1.65 (m, 1H); 1.16 (t, 3H, J=7.1 Hz).

IR (KBr, cm⁻¹) 3300; 3100; 2990; 2900; 1730; 1690; 1600; 1515; 1450; 1350; 1275; 1250; 1175; 1100; 1025; 840; 750; 700.

MS (+FAB) 439.0 (M+H); 422.0; 394.0; 366.0; 176.0; 175.0; 174.0; 165.0; 146.0; 118.0; 106.0; 92.0; 91.0; 90.0; 84.0; 65.0.

Anal. Calcd. for C₂₄H₂₆N₂O₆.0.09 CH₂Cl₂ C, 62.03; H, 5.80; N, 9.59. Found: C, 62.03; H, 5.68; N, 9.40.

### EXAMPLE 595

### Synthesis of N-(Benzyl)-L-pyroglutamyl-L-4-nitrophenylalanine Lithium Salt

N-(Benzyl)-L-pyroglutamyl-L-4-nitrophenylalanine ethyl ester 0.09 methylene chloride solvate (0.100 g, 0.228 mmol) was dissolved in refluxing ethanol (10 mL) and charged to a 25 mL round bottom flask equipped with a magnetic stir bar, air condenser and nitrogen inlet. 1N LiOH (216 μL, 0.216 mmol) was added, via syringe, and the solution stirred at 70° C. for 16 h under nitrogen. The reaction solution went from clear to brown upon the LiOH addition and some precipitate was noted. This solution was transferred to a 125 mL separatory funnel with an additional 50 mL of water and washed with methylene chloride (50 mL). An emulsion formed and was allowed to separate. The organic phase was removed and the aqueous phase was filtered, pumped on for 3 h and lyophilized to give 0.048 g, (53%) of a fluffy white solid, mp=285-287° C. (decompose).

Physical data was as follows:

¹H NMR (DMSO-d₆, 400 MHz) δ 8.07-8.02 (m, 2H); 7.64-7.61 (m, 1H); 7.39-7.36 (m, 2H); 7.31-7.21 (m, 3H); 7.13 (d, 1H, J=7.5 Hz); 7.00 (d, 1H, J=7.6 Hz); 4.75 (d, 1H, J=15.4 Hz); 4.06-4.01 (m, 1H); 3.94-3.89 (m, 1H); 3.45 (d, 1H, J=15.4 Hz); 3.07-2.99 (m, 1H); 2.29-2.14 (m, 2H); 2.12-2.00 (m, 1H); 1.79-1.73 (m, 1H); 1.57-1.54 (m, 1H).

IR (KBr, cm⁻¹) 3400; 3100; 2900; 1675; 1600; 1515; 1450; 1425; 1350; 1250; 1100; 840; 690.

MS (−FAB) 410.1 (M−H); 394.0; 337.1; 275.1; 217.1; 183.0; 153.0; 109.0; 91.0.

Anal. Calcd. for C₂₁H₂₁N₃O₆.1.25H₂O C, 57.34; H, 5.16; N, 9.55. Found: C, 57.44; H, 5.05; N, 9.48.

### EXAMPLE 596

### Synthesis of N-(Benzyl)-L-pyroglutamyl-L-4-aminophenylalanine Ethyl Ester

A solution of N-(benzyl)-L-pyroglutamyl-L-4-nitrophenylalanine ethyl ester 0.09 methylene chloride solvate (0.900 g, 2.048 mmol) and SnCl₂.H₂O in ethanol (30 mL) was charged to a 100 mL round bottom flask equipped with a magnetic stir bar and nitrogen inlet. This solution was stirred at ambient temperature, under nitrogen, for 16 h. The reaction solution was transferred to a 125 mL separatory funnel with 50 mL of ethyl acetate. The organic phase was washed with saturated sodium bicarbonate (50 mL), dried (K₂CO₃), and the solvent removed to yield 0.702 g of a white solid. This material was chromatographed on silica gel (ethyl acetate) giving 0.362 g (39%) of a white solid, mp=147-149° C.

Physical data was as follows:

¹H NMR (DMSO-d₆, 400 MHz) δ 8.49 (d, 1H, J=8.1 Hz); 7.34-7.24 (m, 3H); 7.04 (d, 2H, J=7.7 Hz); 6.84 (d, 2H, J=8.3 Hz); 6.48 (d, 2H, J=9.3 Hz); 4.94 (s, 2H); 4.76 (d, 1H, J=14.9 Hz); 4.44-4.38 (m, 1H); 4.11-4.01 (m, 2H); 3.89-3.86 (m, 1H); 3.40 (d, 1H, J=15.2 Hz); 2.91-2.86 (m, 1H); 2.72-2.66 (m, 1H); 2.34-2.20 (m, 2H); 2.16-2.08 (m, 1H); 1.77-1.71 (m, 1H); 1.16 (t, 3H, J=7.1 Hz).

IR (KBr, cm⁻¹) 3400; 3300; 3040; 3030; 2990; 2950; 1740; 1675; 1550; 1525; 1425; 1225; 1200; 1125; 830; 700.

MS (+FAB) 409.0 (M+H); 336.0; 254.0; 253.0; 191.0; 174.0; 146.0; 107.0; 106.0; 91.0; 90.0; 77.0; 65.0; 55.0; 46.0; 45.0; 44.0.

Anal. Calcd. for C₂₃H₂₇N₃O₄: C, 67.47; H, 6.65; N, 10.26. Found: C, 67.08; H, 6.69; N, 10.20.

### EXAMPLE 597

### Synthesis of N-(Benzyl)-L-pyroglutamyl-L-4-[(thiomorpholin-4′-yl)carbonyloxy]phenylalanine tert-Butyl Ester

A solution of N-benzyl-L-pyroglutamic acid (J. Am. Chem. Soc. 106:4539 (1984), 10.00 g, 45.62 mmol), L-tyrosine tert-butyl ester (11.91 g, 50.17 mmol) and BOP (22.19 g, 50.17 mmol) in DMF (250.0 mL) was charged to a 500 mL round bottom flask equipped with a stir bar and nitrogen inlet. Triethylamine (7.00 mL, 50.17 mmol) was added dropwise and the resulting solution was stirred at 25° C. for 16 h. The solution was transferred to a 1.0 L separatory funnel with ethyl acetate (300 mL) and washed with saturated sodium bicarbonate solution (300 mL×2), brine (300 mL×2), dried (MgSO₄) and the solvent removed to give 18.63 g (93%) of N-(benzyl)-L-pyroglutamyl-L-tyrosine tert-butyl ester as a white solid.

A solution of N-(benzyl)-L-pyroglutamyl-L-tyrosine tert-butyl ester (1.00 g, 2.280 mmol) and 4-nitrophenyl chloroformate (0.442 g, 2.092 mmol) were dissolved in methylene chloride (5 mL) and charged to a 25 mL round bottom flask equipped with a magnetic stir bar and nitrogen inlet. The solution was cooled in an ice bath and triethylamine (729 μL, 5.230 mmol) was added, via syringe, and the resulting yellow solution was stirred for 30 min in an ice bath, then 30 min at ambient temperature. The solution was recooled in an ice bath and thiomorpholine (210 μL, 2.092 mmol) was added. The solution was allowed to warm to room temperature and stirred for 16 h under nitrogen. The solution was transferred to a 250 mL separatory funnel with 100 mL of diethyl ether and this organic phase was washed with 10% K₂CO₃ (50 mL×12), dried (K₂CO₃) and the solvent removed to give a white solid (0.992 g). This material was recrystallized from ethanol to yield 0.411 g (35%) of white crystals, mp=169-171° C.

Physcial data was as follows:

¹H NMR (DMSO-d₆, 400 MHz) δ 8.53 (d, 1H, J=8.1 Hz); 7.33-7.21 (m, 5H); 7.07-7.03 (m, 4H); 4.78 (d, 1H, J=15.2 Hz); 4.49-4.36 (m, 1H); 3.89-3.86 (m, 1H); 3.82 (s, 2H); 3.68 (s, 2H); 3.43 (d, 1H, J=17.0 Hz); 3.08-3.03 (m, 1H); 2.89-2.83 (m, 1H); 2.67 (s, 4H); 2.32-2.26 (m, 2H); 2.17-2.11 (m, 1H); 1.77-1.73 (m, 1H); 1.38 (s, 9H).

IR (KBr, cm⁻¹) 3400; 3300; 3100; 2980; 2910; 1725; 1675; 1660; 1560; 1510; 1460; 1420; 1375; 1300; 1225; 1200; 1100; 960; 800; 760; 700; 650; 550.

MS (+FAB) 635.5 (M+NH₄); 618.4; 562.2; 506.4; 407.8; 344.5; 255.9.

### EXAMPLE 598

### Synthesis of N-(Benzyl)-L-pyroglutamyl-L-4-(1′-benzyloxycarbonylpiperidin-4′-ylcarbonylamino)phenylalanine Methyl Ester

To a solution of N-benzyl-L-pyroglutamic acid (0.50 g, 2.275 mmol), L-4-(1′-benzyloxycarbonylpiperidin-4′-ylcarbonylamino)phenylalanine methyl ester hydrochloride (1.08 g, 2.275 mmol) and BOP (1.10 g, 2.48 mmol) in acetonitrile (60 mL) under nitrogen was added triethylamine (0.7 mL, 5.005 mmol) dropwise. The mixture was stirred 48 h at room temperature. The reaction was then worked-up by evaporation of the solvent, addition of ethyl acetate, sequential washing with 1N HCl solution, water, saturated sodium bicarbonate solution, saturated brine and drying with MgSO₄. Evaporation of the solvent gave a crude solid which was purified by flash chromatography using EtOAc/MeOH (99:1) as eluent, to afford the desired product as a solid (0.332 g, mp 223-225° C., 23% yield).

Physical data was as follows:

¹H NMR (DMSO-d₆, 400 MHz) δ 9.88 (s, 1H); 8.56 (d, 1H, J=8.1 Hz); 7.52 (d, 2H, J=8.5 Hz); 7.4-7.2 (m, 8H); 7.11 (d, 2H, J=8.3 Hz); 6.99 (d, 2H, J=8.1 Hz); 5.07 (s, 2H); 4.73 (d, 1H, J=14.7 Hz); 4.54 (m, 1H); 4.02 (m, 2H); 3.84 (m, 1H); 3.63 (s, 3H); 3.27 (m, 2H); 3.02 (m, 1H); 2.90-2.79 (brd m, 3H); 2.30-2.23 (brd m, 2H); 2.10 (m, 1H); 1.80-1.70 (brd m, 3H); 1.55-1.45 (brd m, 2H).

IR (KBr, cm⁻¹) 3400; 3275; 2910; 1690; 1550; 1435; 1325; 1225; 1120; 1100; 1010; 940; 700.

MS (+FAB) 663.1 ([M+Na]⁺); 597.1; 507.1; 174.0; 91.0.

Anal. Calcd. for C₃₆H₄₀N₄O₇.0.15C₄H₈O₂: C, 66.12; H, 6.35; N, 8.56. Found: C, 66.03; H, 5.01; N, 8.56.

### EXAMPLE 599

### Synthesis of N-(Benzyl)-L-pyroglutamyl-L-4-(1′-benzyloxycarbonylpiperidin-4′-ylcarbonylamino)phenylalanine

To a suspension of N-(benzyl)-L-pyroglutamyl-L-4-(1′-benzyloxycarbonyl-piperidin-4′-ylcarbonylamino)phenylalanine methyl ester (0.30 g, 0.468 mmol) in a aqueous methanol solution (MeOH/H₂O, 12 mL/1 mL) under nitrogen was added solid LiOH (0.039 g, 0.929 mmol). After stirring for 24 h, the solvent was concentrated to about 3 mL and acidified using 10% citric acid solution. A white precipitate was filtered off, washed with water and dried in vacuo to yield the product as a off white solid (0.22 g, mp 193-196° C., yield 75%).

Physical data was as follows:

¹H NMR (DMSO-d₆, 400 MHz) δ 9.87 (s, 1H); 8.42 (d, 1H, J=8.3 Hz); 7.52 (d, 2H, J=8.5 Hz); 7.4-7.2 (m, 8H); 7.11 (d, 2H, J=8.5 Hz); 6.97 (d, 2H, J=7.9 Hz); 5.07 (s, 2H); 4.73 (d, 1H, J=15.1 Hz); 4.49 (m, 1H); 4.02 (brd d, 2H, J=12.7 Hz); 3.84 (m, 1H); 3.26 (s, 1H); 3.07 (m, 1H); 2.98-2.7 (brd m, 3H); 2.32-2.20 (brd m, 2H); 2.10 (m, 1H); 1.80-1.70 (brd m, 3H); 1.55-1.45 (brd m, 2H). IR (KBr, cm⁻¹) 3420; 3300; 3050; 2950; 1660; 1550; 1440; 1325; 1225; 1175; 1110; 1060; 950; 820; 760; 700; 510.

MS (−FAB) 625.4 ([M−H]−); 491.3; 367.2; 275.1; 183.1; 91.0.

Anal. Calcd. for C₃₅H₃₈N₄O₇.1.5H₂O: C, 64.30; H, 6.32; N, 8.57. Found: C, 64.33; H, 6.09; N, 8.44.

### EXAMPLE 600

### Synthesis of N-(Benzyl)-L-pyroglutamyl-L-4-(piperidin-4′-ylcarbonylamino)phenylalanine Hydrobromide

Hydrogen bromide in HOAc (33 wt. %, 2 mL) was added to a flask containing N-(benzyl)-L-pyroglutamyl-L-4-(1′-benzyloxycarbonylpiperidin-4′-ylcarbonylamino)phenylalanine (0.10 g, 0.16 mmol) and stirred for 50 min. Et₂O was added until a precipitate fell out of solution and the mixture was then stirred for 10 min. The precipitate was filtered off and washed with fresh Et₂O. The Et₂O layers were discarded. The precipitate was then washed with water until all of the material on the filter paper dissolved. This aqueous phase was then lyophilized to generate the product as a solid (0.082 g, mp 191-194° C., 81% yield).

Physical data was as follows:

¹H NMR (DMSO-d₆, 400 MHz) δ 9.98 (s, 1H); 8.48 (m, 2H); 8.25 (brd s, 1H); 7.52 (d, 2H, J=8.3 Hz); 7.27-7.20 (m, 3H); 7.13 (d, 2H, J=8.5 Hz); 6.97 (d, 2H, J=8.1 Hz); 4.70 (d, 1H, J=14.9 Hz); 4.49 (m, 1H); 3.83 (m, 1H); 3.05 (m, 1H); 2.86-2.94 (m, 2H); 2.89-2.73 (m, 1H); 2.67-2.57 (m, 1H); 2.32-2.19 (brd m, 2H); 2.14-2.06 (brd m, 1H); 1.98-1.90 (m, 2H); 1.83-1.68 (brd m, 2H).

MS (+FAB) 493.2 ([M+H]⁺); 482.0; 460.1; 307.1; 220.2; 176.0; 154.1.

Anal. Calcd. for C₂₇H₃₂N₄O₅.HBr.3.3H₂O: C, 51.23; H, 6.30; N, 8.85. Found: C, 51.19; H, 5.79; N, 8.80

### EXAMPLE 601

### Synthesis of N-(Benzyl)-L-pyroglutamyl-L-4-(1′-methylpiperidin-4′-yloxy)phenylalanine Ethyl Ester

To a solution of N-(benzyl)-pyroglutamic acid (0.29 g, 1.32 mmol), L-4-(1′-methylpiperidin-4′-yloxy)phenylalanine ethyl ester dihydrochloride salt (0.50 g, 1.32 mmol) and BOP (0.64 g, 1.45 mmol) in DMF (15 mL) under nitrogen was added triethylamine (0.65 mL, 4.62 mmol) and the mixture stirred at room temperature for 7 days. The reaction was quenched by addition of excess saturated sodium bicarbonate solution and EtOAc. The organic phase was separated and concentrated to an oil that was flash chromatographed using CH₂Cl₂/MeOH (95:5) as eluent to afford the product as a solid (0.15 g, 22% yield).

Physical data was as follows:

¹H NMR (DMSO-d₆, 400 MHz) δ 8.57 (d, 1H, J=7.9 Hz); 7.26 (m, 3H); 7.11 (d, 2H, J=8.5 Hz); 7.01 (m, 2H); 6.86 (d, 2H, J=8.5 Hz); 4.72 (d, 1H, J=15.1 Hz); 4.52 (m, 1H); 4.33 (brd s, 1H); 4.0-4.1 (brd m, 3H); 3.85 (m, 1H); 3.02 (m, 1H); 2.70-2.83 (brd m, 3H); 2.40-2.20 (brd m, 7H); 2.12 (m, 1H); 1.93-1.85 (brd s, 2H); 1.75-1.50 (brd m, 4H); 1.21-1.12 (m, 3H).

MS (EI) 507 ([M+H]⁺); 421; 174; 133; 107; 98; 70.

Anal. Calcd. for C₂₉H₃₇N₃O₅: C, 68.62; H, 7.35; N, 8.28. Found: C, 60.53; H, 6.91; N, 8.20.

### EXAMPLE 602

### Synthesis of N-(Benzyl)-L-pyroglutamyl-L-4-(1′-methylpiperidin-4′-yloxy)phenylalanine Lithium Salt

To a solution of N-(benzyl)-L-pyroglutamyl-L-4-(1′-methylpiperidin-4′-yloxy)phenylalanine ethyl ester (0.10 g, 0.197 mmol) in MeOH (3 mL) under nitrogen was added 1N LiOH solution (0.187 mL, 0.187 mmol). After stirring overnight, the solvent was evaporated and 10% citric acid solution was added. A precipitate was filtered off, washed with water and dried in vacuo to produce the product as a solid (0.08 g, m p 232-235° C., 83% yield).

Physical data was as follows:

¹H NMR (DMSO-d₆, 400 MHz) δ 7.60 (d, 1H, J=7.2 Hz); 7.25 (m, 3H); 7.01 (m, 4H); 6.70 (d, 2H, J=11.4 Hz); 4.71 (d, 1H, J=15.1 Hz); 4.18 (m, 1H); 4.02 (m, 1H); 3.87 (m, 1H); 3.05 (m, 1H); 2.85 (m, 1H); 2.57-2.54 (brd m, 2H); 2.28-2.18 (m, 2H); 2.13 (s, 3H); 2.11-2.05 (brd m, 3H); 1.87-1.72 (brd m, 3H); 1.58-1.49 (m, 2H).

MS (+ESI) 480.1 ([M+H]⁺); 352.0; 274.0; 240.9; 179.9.

### EXAMPLE 603

### Synthesis of N-(Benzyl)-L-pyroglutamyl-L-4-(N,N-dimethylcarbamyloxy)phenylalanine Ethyl Ester

To a solution of N-(benzyl)-L-pyroglutamyl-L-tyrosine ethyl ester (0.50 g, 1.22 mmol), dimethylaminopyridine (0.146 g, 1.20 mmol), triethylamine (0.25 mL, 1.83 mmol) and pyridine (1.5 mL) in CH₂Cl₂ (10 mL) was added dimethylcarbamyl chloride (0.15 mL, 1.70 mmol) dropwise. After stirring for 60 h, the reaction was quenched by addition of 10% citric acid solution (40 mL) followed by extraction using ethyl acetate/hexane (65:35) (100 mL). The organic phase was separated and washed sequentially with water, saturated sodium bicarbonate solution, water, saturated brine, dried with MgSO₄ and evaporated in vacuo to afford the product as a solid (0.57 g, mp 150-152° C., 99% yield).

Physical data was as follows:

¹H NMR (DMSO-d₆, 400 MHz) δ 8.59 (d, 1H, J=8.1 Hz); 7.25 (m, 3H); 7.18 (d, 2H, J=8.5 Hz); 7.02 (m, 4H); 4.75 (d, 1H, J=15.3 Hz); 4.53 (m, 1H); 4.10 (m, 2H); 3.85 (m, 1H); 3.40 (d, 1H, J=15.1 Hz); 3.09 (m, 1H); 3.01 (s, 3H); 2.88 (m, 4H); 2.33-2.24 (m, 2H); 2.18-2.08 (m, 1H); 1.76-1.67 (m, 1H); 1.14 (t, 3H, J=7.0 Hz).

IR (KBr, cm⁻¹) 3425; 2900; 1725; 1690; 1660; 1525; 1425; 1380; 1210; 1175; 1010; 845; 800; 750; 650; 520.

MS (EI) 481 ([M+H]⁺); 436; 308; 263; 174; 91; 72.

Anal. Calcd. for C₂₆H₃₁N₃O₆: C, 64.85; H, 6.49; N, 8.73. Found: C, 65.00; H, 6.55; N, 8.70.

### EXAMPLE 604

### Synthesis of N-(Benzyl)-L-pyroglutamyl-L-4-(N,N-dimethylcarbamyloxy)phenylalanine Lithium Salt

To a stirred mixture of N-(benzyl)-L-pyroglutamyl-L-4-(N,N-dimethylcarbamyloxy)phenylalanine ethyl ester (0.3 g, 0.62 mmol) in THF (4 mL) was added 1N LiOH solution (0.59 mL, 0.59 mmol) and the mixture was stirred for 72 h. The reaction was quenched by dilution with water (15-20 mL and the aqueous phase was extracted with CH₂Cl₂ three times. Lyophilization of the aqueous layer produced the product as a solid (0.27 g, 94% yield).

Physical data was as follows:

¹H NMR (DMSO-d₆, 400 MHz) δ 7.54 (d, 1H, J=6.8 Hz); 7.30-7.20 (brd m, 3H); 7.10 (d, 2H, J=8.5 Hz); 7.04 (m, 2H); 6.88 (m, 2H); 4.75 (d, 1H, J=15.1 Hz); 3.99 (q, 1H, J=6.3 Hz); 3.86 (m, 1H); 3.41 (d, 1H, J=15.3 Hz); 3.11 (m, 1H); 3.00 (s, 3H); 2.88 (m, 4H); 2.30-2.19 (brd m, 2H); 2.12-2.05 (m, 1H); 1.79-1.72 (m, 1H).

IR (KBr, cm⁻¹) 3400; 2950; 1660; 1600; 1400; 1220; 1175; 700; 510.

MS (+FAB) 454.0 ([M+H]⁺); 460.0 ([M+Li]⁺); 410.0; 326.9; 279.0; 220.9; 173.9; 130.6; 80.3.

Anal. Calcd. for C₂₄H₂₇N₃O₆Li.3H₂O: C, 56.14; H, 6.28; N, 8.18. Found: C, 56.07; H, 5.88; N, 7.95.

### EXAMPLE 605

### Synthesis of N-(Benzyl)-L-pyroglutamyl-L-4-(N,N-dimethylcarbamyloxy)phenylalanine tert-Butyl Ester

BOP coupling of N-(benzyl)-L-pyroglutamic acid (1.96 g, 8.9 mmol) and L-tyrosine tert-butyl ester (2.32 g, 9.78 mmol) with triethylamine in DMF according to the method of Example 597 followed by a saturated sodium bicarbonate quench, addition of EtOAc and extraction with 10% citric acid solution, water, brine, drying (MgSO₄), filtration and concentration produced the precursor N-(benzyl)-L-pyroglutamyl-L-tyrosine acid tert-butyl ester (3.59 g, mp 167-169° C., 92% yield) as a crystalline solid.

Physical data was as follows:

Anal. Calcd. for C₂₅H₃₀N₂O₅: C, 68.48; H, 6.90; N, 6.39. Found: C, 68.20; H, 6.78; N, 6.75.

To a combined mixture of N-(benzyl)-L-pyroglutamyl-L-tyrosine acid tert-butyl ester (0.5 g, 1.14 mmol), N,N-dimethylaminopyridine (0.14 g, 1.14 mmol) and triethylamine (0.24 mL, 1.71 mmol) in CH₂Cl₂ (8 mL) was added dimethylcarbamyl chloride (0.15 mL, 1.59 mmol) dropwise. After stirring for 66 h, the reaction was quenched by addition of 10% citric acid solution (30 μL) followed by extraction using ethyl acetate/hexane (65:35) mixture (100 mL). The organic phase was separated, washed sequentially with water, saturated sodium bicarbonate solution, water, saturated brine, dried with MgSO₄ and evaporated in vacuo to afford the product as a solid (0.52 g, mp 184-185° C., 90% yield).

Physical data was as follows:

¹H NMR (DMSO-d₆, 400 MHz) δ 8.52 (d, 1H, J=8.1 Hz); 7.32-7.20 (m, 5H); 7.05-7.01 (m, 4H); 4.76 (d, 1H, J=15.1 Hz); 4.46 (m, 1H); 3.88 (m, 1H); 3.40 (d, 1H, J=15.1 Hz); 3.05-3.0 (m, 4H); 2.90 (s, 3H); 2.85 (m, 1H); 2.34-2.26 (m, 2H); 2.18-2.11 (m, 1H); 1.78-1.71 (m, 1H); 1.37 (s, 9H).

IR (KBr, cm⁻¹) 3410; 3275; 2950; 1725; 1660; 1550; 1430; 1375; 1210; 1150; 750; 690; 520.

MS (EI) 509 ([M+H]⁺); 453; 408; 233; 174; 91; 72.

Anal. Calcd. for C₂₈H₃₅N₃O₆: C, 66.00; H, 6.92; N, 8.25. Found: C, 65.88; H, 6.91; N, 8.24.

### EXAMPLE 606

### Synthesis of N-(Benzyl)-L-pyroglutamyl-L-4-[(4′-methylpiperazin-1′-yl)carbonyloxy]phenylalanine tert-Butyl Ester

N-(benzyl)-L-pyroglutamyl-L-tyrosine acid tert-butyl ester (0.50 g, 1.14 mmol) was combined with p-nitrophenyl chloroformate (0.218 g, 1.08 mmol) in CH₂Cl₂ (10 mL) and the reaction mixture was cooled to 0° C. under N₂. Triethylamine (0.4 mL, 2.85 mmol), previously dissolved in 2 mL CH₂Cl₂, was added dropwise to the mixture and stirred 30 min at 0° C. The mixture is then brought to ambient temperature and stirred 30 min followed by a recooling to 0° C. and addition of 1-methyl-piperazine (0.12 mL, 1.08 mmol). The reaction mixture was then allowed to warm to room temparature and stirred 66 h. The reaction was quenched by dilution with Et₂O and washed sequentially with 10% K₂CO₃ solution (5×) and 1N HCl. The acid layer was removed and the pH adjusted to 8 using saturated sodium bicarbonate solution. Extraction of the aqueous phase with EtOAc and followed by brine wash, drying over MgSO₄, evaporation and recrystallization (CH₂Cl₂/hexane) produced the product as a solid (0.372 g, mp 113-116° C., 58% yield).

Physical data was as follows:

¹H NMR (DMSO-d₆, 400 MHz) δ 8.52 (d, 1H, J=8.1 Hz); 7.30-7.20 (m, 5H); 7.03 (m, 4H); 4.76 (d, 1H, J=15.1 Hz); 4.46 (m, 1H); 3.88 (m, 1H); 3.58 (brd s, 2H); 3.41 (m, 3H); 2.38-2.32 (brd s, 4H); 2.22 (s, 3H); 1.78-1.70 (m, 1H); 1.37 (s, 9H).

IR (KBr, cm⁻¹) 3410; 3275; 2925; 1725; 1690; 1660; 1550; 1475; 1350; 1290; 1220; 1150; 1050; 1000; 850; 700; 510.

MS (+ESI) 565.5 ([M+H]⁺); 509.2; 475.5; 344.1; 279.1; 221.0.

Anal. Calcd. for C₃₁H₄₀N₄O₆. 0.1 CH₂Cl₂: C, 64.96; H, 7.06; N, 9.77. Found: C, 64.93; H, 7.10; N, 9.62.

10.5 Synthesis of Compounds of PEG Derivatives

The following methods may be used to prepare the compounds of this invention. In one method outlined in Scheme 17 below is illustrative of such preparation.

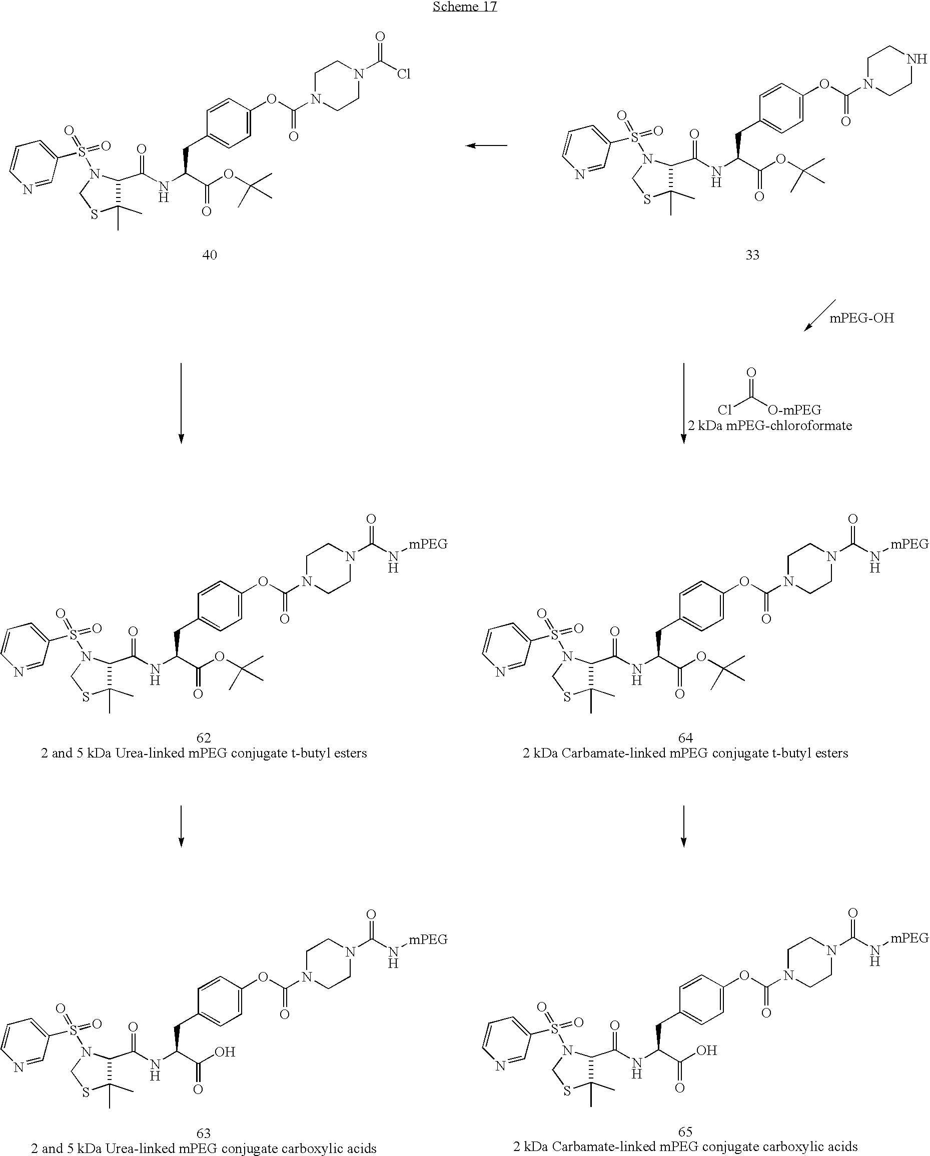

The following Examples describe methods for preparing the compounds shown in Scheme 6 and Scheme 17 above. Unless otherwise indicated some or all of the following HPLC methods were used in the preparation of the following exemplary compounds.

Method A1: Samples of conjugates of more than 100 mg were purified using reverse phase HPLC on a Phenomenex Luna C18(2), 5 μm column 250 mm×21.2 mm with a Varian IV detector, using a gradient of 40-60% ACN+0.1% TFA in 100 min at 15 mL/min.

Method B1: Samples of conjugates of more than 100 mg but less than 500 mg were purified using reverse phase HPLC on a Phenomenex Luna C18(2), 10 μm column 250 mm×50 mm with a Varian IV detector using a gradient of 40-60% ACN+0.1% TFA in 100 min at 60 mL/min.

Method C1: The purity of conjugates was confirmed using reverse phase HPLC on a Luna 3 μm C18(2) column (30×4.6 mm) with a Sedex 75 (35° C., gain=5) evaporative light scattering detector, using a gradient of 20-70% ACN w/0.1% TFA at a flow rate of 1.5 mL/min.

### EXAMPLE 607

### Preparation of 2 kDa urea-linked mPEG conjugate carboxylic acid

Step 1: Preparation of Compound 29

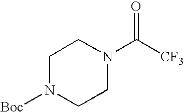

Compound 25 (20 g, 0.11 mol) (as shown in Scheme 6 above) was dissolved in CH₂Cl₂ (500 mL) under N₂. The reaction mixture was cooled to 0° C. Triethylamine (18.12 mL, 0.13 mol) was added, followed by trifluoroacetic anhydride (18.14 mL, 0.13 mol) in portions. The reaction was allowed to warm to room temperature overnight. The reaction mixture was concentrated in vacuo and the residue was taken up in ethyl acetate (200 mL). The organic phase was washed with H₂O, sat. NaHCO₃, brine, dried over Na₂SO₄, filtered, and concentrated in vacuo to yield 29.73 g (96%) of the title compound, 29, as a yellow solid.

¹H NMR (CDCl₃) δ 3.64-3.60 (m, 2H), 3.55-3.53 (m, 2H), 3.49-3.45 (m, 4H), 1.44 (s, 9H).

¹³C NMR (CDCl₃) δ 155.7 (J_{C-F}=36 Hz), 154.3, 116.4 (J_{C-F}=288 Hz), 80.8, 45.7, 43.3, 28.3.

### Step 2: Preparation of Compound 30

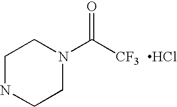

Compound 29 (29.26 g, 0.10 mol) was added in portions to a 500 mL flask containing a solution of 4N HCl in dioxane (200 mL) at 0° C. The reaction was stirred in ice bath for 4 hours when TLC (3:1 hexanes: ethyl acetate) showed 100% conversion to product. The reaction mixture was concentrated in vacuo and treated with ethyl ether (500 mL). The product was filtered and dried to yield 22.53 g (99%) compound 30 as a white mono-hydrochloride salt.

¹H NMR (DMSO-d₆) δ 3.82-3.79 (m, 4H), 3.53 (s, 1H), 3.18-3.16 (m, 4H).

¹³C NMR (DMSO-d₆) δ 154.3 (J_{C-F}=35 Hz), 115.9 (J_{C-F}=289 Hz), 66.1, 42.0, 41.9, 41.5.

### Step 3: Preparation of Compound 31

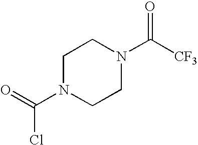

A 250 mL flask was charged with compound 30 (1.0 g, 4.6 mmol), CH₂Cl₂ (40 mL), and sat. NaHCO₃ (40 mL). The reaction mixture was stirred vigorously at 0° C. for 15 minutes. Stirring was ceased and the layers were allowed to separate. A 2.0 M solution of phosgene in toluene (9 mL, 18 mmol) was added to the reaction mixture which was stirred vigorously for 30 minutes, while maintaining temperature at 0° C. The layers were separated and the aqueous phase was washed with CH₂Cl₂ (15 mL). The combined organic layers were washed with brine, dried over Na₂SO₄, filtered, and concentrated in vacuo. The residue was taken up in CH₂Cl₂ and concentrated in vacuo again to yield 1.04 g (92%) compound 31 as a white solid.

MS (PI-FAB) 245, (M+H)⁺.

¹H NMR (CDCl₃) δ 3.80-3.68 (m, 8H).

¹³C NMR (CDCl₃) δ 155.9 (J_{C-F}=37 Hz), 148.7 (J_{C-F}=12 Hz), 116.3 (J_{C-F}=289 Hz), 48.3, 47.8, 45.7, 45.3, 45.1, 42.9, 42.7.

### Step 4: Preparation of Compound 32

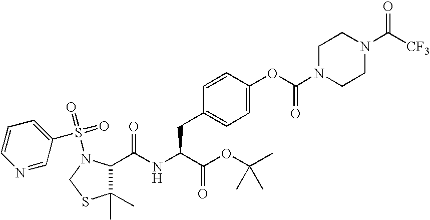

A 25 mL flask was charged with compound 24 (5.97 g, 0.011 mol), DMAP (1.34 g, 0.011 mol), and CH₂Cl₂ (22 mL). Triethylamine (2.4 mL, 0.017 mol) was added followed by compound 31 (4.2 g, 0.017 mol). The reaction mixture was heated at reflux for 20 hours. The reaction mixture was concentrated in vacuo and the residue was taken up in ethyl acetate. The organic phase was washed with sat. NaHCO₃, H₂O, brine, dried over Na₂SO₄, filtered, and concentrated in vacuo to yield 9.31 g (115%) pink foam. The crude material was purified by flash chromatography (gradient of 50% ethyl acetate/hexanes to 75% ethyl acetate/hexanes) to yield 6.1 g (76%) compound 32 as a pale pink foam. R^{f}=0.14 (1:1 hexanes:ethyl acetate).

MS (PI-FAB) 730, (M+H)⁺.

¹H NMR (CDCl₃) δ 9.08-9.07 (m, 1H), 8.87-8.85 (m, 1H), 8.16-8.14 (m, 1H), 7.52-7.48 (m, 1H), 7.25-7.22 (d, 2H), 7.03-7.00 (d, 2H), 6.91-6.88 (d, 1H), 4.78-4.70 (q, 1H), 4.60-4.44 (dd, 2H), 3.88 (s, 1H), 3.75-3.60 (m, 8H), 3.09-3.06 (m, 2H), 1.42 (s, 9H), 1.18 (s, 3H), 1.16 (s, 3H).

### Step 5: Preparation of Compound 33

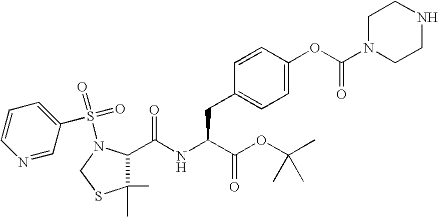

To a solution of compound 32 (6.11 g, 8.4 mmol) dissolved in MeOH (90 mL) was added a solution of potassium carbonate (5.79 g, 42 mmol) in H₂O (10 mL). The reaction was stirred at room temperature for 15 minutes and then concentrated in vacuo. The residue was filtered and washed with copious amounts of H₂O to yield 4.65 g (88%) compound 33 as a white solid. R^{f}=0.08 (5% MeOH/CH₂Cl₂).

MS (PI-FAB) 634, (M+H)⁺.

¹H NMR (CDCl₃) δ 9.09-9.08 (m, 1H), 8.87-8.85 (m, 1H), 8.16-8.14 (m, 1H), 7.52-7.48 (m, 1H), 7.23-7.20 (d, 2H), 7.03-7.00 (d, 2H), 6.91-6.88 (d, 1H), 4.78-4.70 (q, 1H), 4.59-4.46 (dd, 2H), 3.89 (s, 1H), 3.65-3.50 (m, 4H), 3.09-3.06 (m, 2H), 2.92-2.88 (m, 4H), 1.43 (s, 9H), 1.19 (s, 3H), 1.17 (s, 3H).

¹³C NMR (CDCl₃) 170.1, 167.9, 154.5, 153.9, 150.7, 148.8, 136.0, 133.4, 133.2, 130.6, 124.1, 121.9, 83.0, 73.9, 55.0, 53.7, 50.7, 46.0, 45.7, 45.0, 37.9, 29.3, 28.0, 24.0.

### Step 6: Preparation of Compound 40

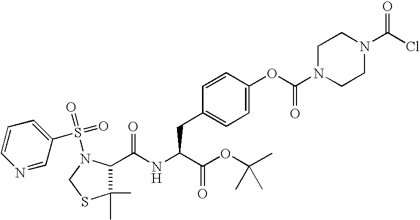

A 250 mL flask was charged with compound 33 (2.5 g, 3.9 mmol), CH₂Cl₂ (40 mL), and sat. NaHCO₃ (40 mL). The reaction mixture was stirred vigorously at 0° C. for 15 minutes. Stirring was ceased and the layers were allowed to separate. A 2.0 M solution of phosgene in toluene (7.9 mL, 16 mmol) was quickly added to the reaction mixture which was stirred vigorously for 60 minutes maintaining the temperature at 0° C. The layers were separated and the aqueous phase was washed with CH₂Cl₂ (30 mL). The combined organic layers were washed with 0.2 N citric acid, brine, dried over Na₂SO₄, filtered, and concentrated in vacuo to yield 2.76 g (100%) white foam. The crude material was purified through a silica plug, eluting with 100% ethyl acetate, to yield 2.15 g (78%) compound 40 as a white foam. R_{f}=0.43 (3:1 ethyl acetate: hexanes).

¹H NMR (CDCl₃) δ 9.09-9.08 (m, 1H), 8.87-8.85 (m, 1H), 8.16-8.14 (d, 1H), 7.52-7.48 (m, 1H), 7.25-7.22 (d, 2H), 7.03-7.01 (d, 2H), 6.90-6.88 (d, 1H), 4.78-4.70 (q, 1H), 4.60-4.45 (dd, 2H), 3.88 (s, 1H), 3.79-3.65 (m, 8H), 3.10-3.07 (m, 2H), 1.43 (s, 9H), 1.18 (s, 3H), 1.17 (s, 3H).

¹³C NMR (CDCl₃) δ 169.9, 167.9, 154.1, 153.6, 150.2, 148.5, 136.1, 133.8, 130.6, 124.2, 121.7, 82.9, 73.7, 54.8, 53.8, 50.6, 48.3, 45.8, 37.7, 29.2, 27.9, 23.9.

### Step 7: Preparation 2 kDa urea-linked mPEG conjugate t-butyl ester

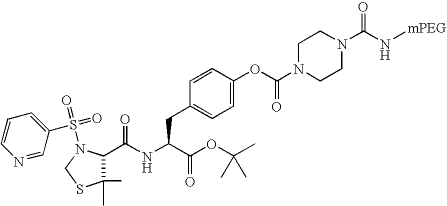

The 2 kilodalton mPEG-amine (192 mg, 0.09 mmol) and DMAP (11 mg, 0.09 mmol) were dissolved in CH₂Cl₂ (0.6 mL). Triethylamine (19.5 μL, 0.14 mmol) was added, followed by compound 40 (100 mg, 0.14 mmol). The reaction mixture was heated to reflux for 20 hours. The reaction was concentrated in vacuo and the residue was taken up in MeOH (25 mL). 2% cross-linked polystyrene sulfonic acid resin (300 mg) was added and reaction vessel was swirled for 2 hours. The mixture was then filtered and concentrated in vacuo to yield 182 mg (˜50%) of a beige solid which was purified by HPLC Method B1 yielding 50.7 mg 2 kDa mPEG conjugate t-butyl ester as a white wax. R_{f}=0.12 (5% MeOH/CH₂Cl₂). HPLC Method C1 determined conjugate to be >99% pure with no remaining compound 33 or mPEG-amine (retention time=1.924).

¹H NMR (CDCl₃) δ 8.21-8.18 (d, 1H), 7.23-7.21 (d, 2H), 7.03-7.00 (d, 2H), 6.91-6.88 (d, 1H), 4.76-4.73 (q, 1H), 4.60-4.46 (dd, 2H), 3.91-3.86 (m, 3H), 3.64 (bs, 184H), 3.37 (s, 3H), 3.09-3.06 (m, 3H), 1.43 (s, 9H), 1.20 (s, 3H), 1.17 (s, 3H).

### Step 8: Preparation 2 kDa urea-linked mPEG conjugate carboxylic acid

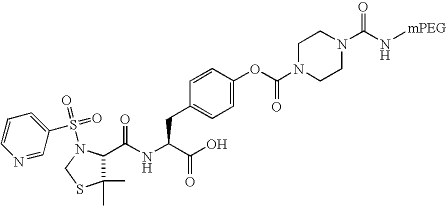

The 2 kDa urea-linked mPEG conjugate t-butyl ester (94 mg, 0.04 mmol) was dissolved in formic acid (5 mL) and heated at 40° C. for 48 hours. The reaction was concentrated in vacuo to yield 88 mg (100%) beige gel, which was purified by HPLC Method A1 to yield 53.7 mg (˜60%) of the free carboxylic acid as a white wax. R_{f}=0.45 (7/3 MeOH:H₂₀+0.1% TFA; C-18 Reverse Phase). HPLC Method C1 determined conjugate to be >99% pure (retention time=2.188)

¹H NMR (CDCl₃) δ 9.07 (bs, 1H), 8.86-8.85 (m, 1H), 8.23-8.20 (d, 1H), 7.59-7.55 (m, 1H), 7.26-7.21 (d, 2H), 7.02-6.96 (m, 2H), 4.82-4.80 (m, 1H), 4.60-4.49 (dd, 2H), 3.99 (s, 1H), 3.62 (bs, 184H), 3.37 (s, 3H), 3.15-3.13 (m, 2H), 1.25 (s, 3H), 1.23 (s, 3H).

### EXAMPLE 608

### Preparation of 5 kDa urea-linked mPEG conjugate Carboxylic Acid

The 5 kDa urea-linked mPEG conjugate t-butyl ester was prepared in the same manner as the 2 kDa conjugate above, using a 5 kDa mPEG-amine, and yielded 476 mg (˜90%) white solid. The crude material (200 mg, 0.04 mmol) was deprotected in the same manner as above yielding 182 mg (100%) beige gum. This was purified by HPLC Method B1, yielding 74.5 mg of the 5 kDa urea-linked MPEG conjugate carboxylic acid as a white powder. R_{f}=0.16 (7/3 MeOH:H₂₀+0.1% TFA; C-18 Reverse Phase). HPLC Method C1 determined conjugate to be >99% pure (retention time=2.260).

¹H NMR (CDCl₃) δ 9.07 (bs, 1H), 8.86-8.85 (m, 1H), 8.17-8.15 (d, 1H), 7.54-7.50 (m, 1H), 7.26-7.22 (d, 2H), 7.03-7.00 (d, 2H), 6.95-6.93 (d, 1H), 5.46 (bs, 1H), 4.83-4.81 (m, 1H), 4.60-4.46 (dd, 2H), 3.93 (s, 1H), 3.64 (bs, 490H), 3.37 (s, 3H), 3.16 (m, 3H), 1.22 (s, 6H).

### EXAMPLE 609

### Preparation of 2 kDa carbamate-linked mPEG conjugate t-butyl ester

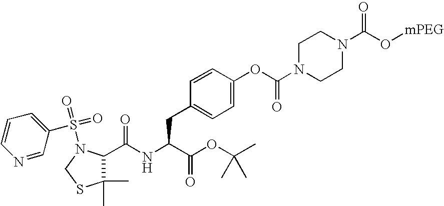

The carbamate linked conjugates were prepared based on a method modified from International Patent Publication Number WO 92/16555. Thus, a 2 kDa mPEG-alcohol (500 mg, 0.25 mmol) was dried by azeotropic distillation in toluene (5 mL). The solution was cooled to room temperature and CH₂Cl₂ (5 mL) was added, followed by a 2.0 M solution of phosgene in toluene (0.38 mL, 0.75 mmol). The reaction was stirred at room temperature for 18 hours and then concentrated in vacuo to yield 500 mg (100%) of the 2 kDa mPEG chloroformate as a white solid. A solution of compound 33 (317 mg, 0.5 mmol) in CH₂Cl₂ (3 mL) was added to the 2 kDa mPEG chloroformate (500 mg, 0.25 mmol) dissolved in CH₂Cl₂ (2 mL). Triethylamine (35 μL, 0.25 mmol) was added and reaction was stirred at room temperature for 30 minutes. The reaction mixture was concentrated in vacuo and the residue was taken up in MeOH (10 mL). 2% cross-linked polystyrene sulfonic acid resin (750 mg) was added and the reaction vessel was swirled for 2 hours. The mixture was then filtered and concentrated in vacuo to yield 470 mg (75%) of the 2 kDa carbamate-linked mPEG conjugate t-butyl ester as a white solid. HPLC Method C1 shows >96% pure (retention time=2.639).

### EXAMPLE 610

### Preparation of 2 kDa carbamate-linked mPEG conjugate carboxylic acid

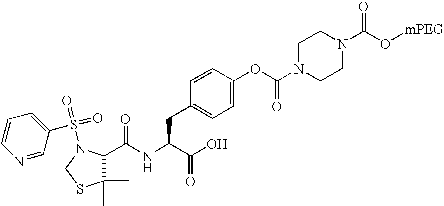

The crude 2 kDa carbamate-linked mPEG conjugate t-butyl ester (250 mg, 0.1 mmol) was dissolved in formic acid (5 mL) and heated at 40° C. for 48 hours. The reaction was concentrated in vacuo to yield 280 mg (100%) of the 2 kDa carbamate-linked MPEG conjugate carboxylic acid as a beige gel.

The following Methods may be used to test compounds of this invention.

### EXAMPLE A

### α⁴β¹ Integrin Adhesion Assay: Jurkat™ Cell Adhesion to Human Plasma Fibronectin

Procedure:

96 well plates (Costar 3590 EIA plates) were coated with human fibronectin (Gibco/BRL, cat #33016-023) at a concentration of 10 μg/mL overnight at 4° C. The plates were then blocked with a solution of bovine serum albumin (BSA; 0.3%) in saline. Jurkat™ cells (maintained in log phase growth) were labeled with Calcein AM according to the manufacturer's instructions, and suspended at a concentration of 2×10⁶ cells/mL in Hepes/Saline/BSA. The cells were then exposed to test and control compounds for 30 minutes at room temperature before transfer to individual wells of the fibronectin coated plate. Adhesion was allowed to occur for 35 minutes at 37° C. The wells were then washed by gentle aspiration and pipetting with fresh saline. Fluorescence associated with the remaining adherent cells was quantified using a fluorescence plate reader at EX 485/EM 530.

Cell cultures were prepared by first splitting the stationary phase Jurkat™ cells at 1:10 on day one, and 1:2 on day two to perform assay on day 3. The cells split 1:10 on day one were split 1:4 on day 3 for a day 4 assay.

The assay plates were prepared by first making a working solution of Gibco/BRL Human Fibronectin (cat # 33016-023) in PBS++, at 10 μg/mL. A Costar 3590 EIA plate was then coated with 50 μL/well for 2 hours at room temperature (thought it can also be left overnight at 4° C.). Finally the plate was asperated and blocked with Hepes/Saline Buffer, 100 μL/well, for 1 hour at RT followed by washing 3× with 150 μL of PBS++.

Compound dilutions were accomplished by preparing 1:3 serial dilutions of compounds as follows. For each plate (4 compounds/plate) 600 μL were added to 4 Bio-Rad Titertubes in a Titertube rack. Enough compound was added to each appropriate tube to give a 2× concentration using methods well known in the art. Using Falcon Flexiplates, 100 μL of Hepes/Saline buffer or human serum were added to rows B through G. A multi-channel pipetter set to 180 μL was used to with four tips spaced evenly the pipetter. Each set of four tubes was mixed 5 times and 180 μL of 2× compound was transferred to the first column of each compound dilution in Row B, leaving Row A empty. 180 μL were added to the other wells in Row A. Serial dilutions were performed down the plate by transferring 50 μL to the next dilution and mixing 5 times, changing tips each time after mixing. Dilutions were stopped at Row F. Row G had no compound present.

A 20 μg/mL solution in Hepes/Saline buffer or human serum, of 21/6 antibody was the positive control and was set aside in a reagent trough to add to cell suspension plate.

The cell staining was accomplished by first harvesting the log-phase Jurkat™ cells by centrifugation in 50 mL tubes (1100 rpm for 5 minutes). The cells were resuspended in 50 mL PBS++, spun, and resuspend in 20 mL PBS++. The cells were stained by adding 20 μL of Calcein AM for 30 minutes R.T. The volume was brought to 50 mL with Hepes/Saline buffer and the cells were counted, spun, and resuspend to 2×10⁶ cells/mL in Hepes/Saline buffer or human serum.

The compounds were incubated using the following procedure. In a new flexiplate, 65 μL of stained cells were added to Rows B through H. Then 65 μL of 2× compounds were added to the appropriate rows following the plate setup and mixed 3×. 65 μL of 2×-21/6 antibody were added to Row H and mixed 3×. Finally the plate was incubated at room temperature for 30 minutes.

Fibronectin adhesion was measured using a fluorescent plate reader at EX 485/EM 530 after the following work up procedure. After incubation, the cells were mixed 3× and 100 μL were transfered to the Fibronectin coated plates and incubated at 37° C. for about 35 minutes. Each plate was washed, row by row, by gently pipetting 100 μL of R.T. PBS++ down the sides of the wells and turning the plate 90 degrees to aspirate. This procedure was repeated for a total of 3 washes. Each well was filled with 100 μL after washing by pipetting down the side of the well.

### EXAMPLE B

### In Vitro Saturation Assay for Determining Binding of Candidate Compounds to α₄ μl

The following describes an in vitro assay to determine the plasma levels needed for a compound to be active in the Experimental Autoimmune Encephalomyelitis (“EAt”) model, described in the next example, or in other in vivo models.

Log-growth Jurkat cells are washed and resuspended in normal animal plasma containing 20 μg/mL of the 15/7 antibody (described in the above example).

The Jurkat cells are diluted two-fold into either normal plasma samples containing known candidate compound amounts in various concentrations ranging from 66 μM to 0.01 μM, using a standard 12 point serial dilution for a standard curve, or into plasma samples obtained from the peripheral blood of candidate compound-treated animals.

Cells are then incubated for 30 minutes at room temperature, washed twice with phosphate-buffered saline (“PBS”) containing 2% fetal bovine serum and 1 mM each of calcium chloride and magnesium chloride (assay medium) to remove unbound 15/7 antibody.

The cells are then exposed to phycoerythrin-conjugated goat F(ab′)₂ anti-mouse IgG Fc (Immunotech, Westbrook, Me.), which has been adsorbed for any non-specific cross-reactivity by co-incubation with 5% serum from the animal species being studied, at 1:200 and incubated in the dark at 4° C. for 30 minutes.

Cells are washed twice with assay medium and resuspended in the same. They are then analyzed with a standard fluorescence activated cell sorter (“FACS”) analysis as described in Yednock et al. J. Biol. Chem., 1995, 270:28740.

The data is then graphed as fluorescence versus dose, e.g., in a normal dose-response fashion. The dose levels that result in the upper plateau of the curve represent the levels needed to obtain efficacy in an in vivo model.

This assay may also be used to determine the plasma levels needed to saturate the binding sites of other integrins, such as the α₉β₁ integrin, which is the integrin most closely related α₄β₁ (Palmer et al, 1993, J. Cell Bio., 123:1289). Such binding is predictive of in vivo utility for inflammatory conditions mediated by α₉β₁ integrin, including by way of example, airway hyper-responsiveness and occlusion that occurs with chronic asthma, smooth muscle cell proliferation in atherosclerosis, vascular occlusion following angioplasty, fibrosis and glomerular scarring as a result of renal disease, aortic stenosis, hypertrophy of synovial membranes in rheumatoid arthritis, and inflammation and scarring that occur with the progression of ulcerative colitis and Crohn's disease.

Accordingly, the above-described assay may be performed with a human colon carcinoma cell line, SW 480 (ATTC #CCL228) transfected with cDNA encoding α₉ integrin (Yokosaki et al., 1994, J. Biol. Chem., 269:26691), in place of the Jurkat cells, to measure the binding of the α₉β₁ integrin. As a control, SW 480 cells which express other α and β₁ subunits may be used.

Accordingly, another aspect of this invention is directed to a method for treating a disease in a mammalian patient, which disease is mediated by α₉β₁ and which method comprises administering to said patient a therapeutically effective amount of a compound of this invention. Such compounds are preferably administered in a pharmaceutical composition described herein above. Effective daily dosing will depend upon the age, weight, condition of the patient which factors can be readily ascertained by the attending clinician. However, in a preferred embodiment, the compounds are administered from about 20 to 500 μg/kg per day.

### EXAMPLE C

### Cassette Dosing and Serum Analysis for Determination of Bioavailability

The oral bioavailability was screened by dosing rats with a cassette, i.e. mixture of 6 compounds per dosing solution. The cassette included 5 test articles and a standard compound, for a total dose of 10 mg/kg. Each compound/test article was converted to the sodium salt with equimolar 1 N NaOH and dissolved in water at 2 mg/mL. The cassette was prepared by mixing equal volumes of each of the six solutions. The cassette dosing solution was mixed well and then the pH was adjusted to 7.5-9. The dosing solution was prepared the day before the study and stirred overnight at room temperature.

Male Sprague Dawley (SD) rats from Charles River Laboratories, 6-8 weeks old were used in this screen. Rats were quarantined for at least one day and had continuous access to food and water. On the night before the administration of the cassette, the rats were fasted for approximately 16 hours.

Four SD rats were assigned in each cassette. A single dose of the dosing solution was administered orally to each rat. The dosing volume (5 mL/kg) and time were recorded and rats were fed 2 hours after dosing.

Blood samples were collected via cardiac puncture at the following time points: 4 hour, 8 hour and 12 hour. Immediately prior to blood collection, rats were anesthetized with CO₂ gas within 10-20 seconds. After the 12-hour samples were collected, the rats were euthanized via CO₂ asphyxiation followed by cervical dislocation.

Blood samples were kept in heparinized microtainer tubes under sub-ambient temperature (4° C.) before they were processed. Blood samples were centrifuged (10000 rpm for 5 minutes) and plasma samples were removed and stored in a −20° C. freezer until analyzed for drug levels. Drug levels in the plasma were analyzed using the following protocol for direct plasma precipitation.

The in vivo plasma samples were prepared in a 1.5 mL 96-well plate, by adding, in order, 100 μL of the test plasma, 150 μL of methanol, followed by vortexing for 10-20 seconds. 150 μL of 0.05 ng/μL of an Internal Standard in acetonitrile were added and vortexed for 30 seconds.

The standard curve samples were prepared in a 1.5 mL 96-well plate, by adding, in order, 100 μL of control mouse plasma, followed by 150 μL of methanol and vortexing for 10-20 seconds. 150 μL of 0.05 ng/μL of an Internal Standard in acetonitrile were added and vortexed for 30 seconds. The samples were spiked with 0-200 ng (10 concentrations) of the compound of interest in 50% methanol to obtain a standard curve range of 0.5 ng/mL-2,000 ng/mL. Again, the sample was vortexed for 30 seconds.

The samples were then spun for 20-30 min at 3000 rpm in an Eppendorf microfuge before 80-90% of supernatant was transferred into a clean 96-well plate. The organic solvent was then evaporated until the samples were dry (under N₂ at 40° C./30-60 min (ZymarkTurbovap)).

The residue was then dissolved in 200-600 L mobile phase (50% CH₃OH/0.1% TFA). LC/MS/MS was then run using a PE-Sciex API-3000 triple quadurpole mass spectrometer (SN0749707), Perkin-Elmer, Series200auto-sampler, and shimadzu 10A pump. Acquisition was done with PE-Sciex Analyst (v1.1) and data analysis and quantification were accomplished using PE-Sciex Analyst (v1.1). A 5-50 μL sample volume was injected onto a reverse phase ThermoHypersil DASH-18 column (Keystone 2.0×20 mm, 5 μm, PN: 8823025-701) using a mobile phase of 25% CH₃OH, 0.1% TFA-100% CH₃OH, 0.1% TFA. The run time was about 8 min at a flow rate of about 300 μL/min.

The Area Under the Curve (AUC) was calculated using the linear trapezoidal rule from t=0 to the last sampling time tₓ (see Handbook of Basic Pharmacokinetics, Wolfgang A. Ritschel and Gregory L. Kearns, 5^{th} ed, 1999).
AUC^{0-tx}=C((Cₙ+Cₙ₊₁)/2))C(tₙ₊₁−tₙ) [(μg/mL)h]

In the case of the cassette dosing paradigm, samples at 4, 8 and 12 h post extravascular dosing, the AUC was calculated from t=0 to t=12 h. The AUC^{0→12 h} values were calculated for each individual animal and the average AUC^{0→12 h} was reported.

### EXAMPLE D

### Asthma Models

Inflammatory conditions mediated by α₄β₁ integrin include, for example, eosinophil influx, airway hyper-responsiveness and occlusion that occurs with chronic asthma. The following describes animal models of asthma that were used to study the in vivo effects of the compounds of this invention for use in treating asthma.

Rat Asthma Model

Following the procedures described by Chapman et al, Am J. Resp. Crit. Care Med,. 153 4, A219 (1996) and Chapman et al, Am. J. Resp. Crit. Care Med 155:4, A881 (1997), both of which are incorporated by reference in their entirety. Ovalbumin (OA; 10 Cg/mL) were mixed with aluminum hydroxide (10 mg/mL) and injected (i.p.) in Brown Norway rats on day 0. Injections of OA, together with adjuvant, were repeated on days 7 and 14. On day 21, sensitized animals were restrained in plastic tubes and exposed (60 min) to an aerosol of OA (10 mg/kg) in a nose-only exposure system. Animals will be sacraficed 72 hours later with pentobarbital (250 mg/kg, i.p.). The lungs were lavaged via a tracheal cannula using 3 aliquots (4 mL) of Hank's solution (HBSS×10, 100 mL; EDTA 100 mM, 100 mL; HEPES 1 M, 25 mL; made up to 1 L with H₂O); recovered cells were pooled and the total volume of recovered fluid adjusted to 12 mL by addition of Hank's solution. Total cells were counted (Sysmex microcell counter F-500, TOA Medical Electronics Otd., Japan) and smears were made by diluting recovered fluid (to approximately 10⁶ cells/mL) and pipetting an aliquot (100 μL) into a centrifuge (Cytospin, Shandon, U.K.). Smears were air dried, fixed using a solution of fast green in methanol (2 mg/mL) for 5 seconds and stained with eosin G (5 seconds) and thiazine (5 seconds) (Diff-Quick, Browne Ltd. U.K.) in order to differentiate eosinophils, neutrophils, macrophages and lymphocytes. A total of 500 cells per smear were counted by light microscopy under oil immersion (×100). Compounds of this invention were formulated into a 0.5% carboxymethylcellulose and 2% Tween80 suspension and administered orally to rats which had been sensitized to the allergen, ovalbumin. Compounds which inhibited allergen-induced leucocyte accumulation in the airways of actively sensitized Brown Norway rats were considered to be active in this model.

Mouse Asthma Model

Compounds were also evaluated in a mouse model of acute pulmonary inflammation following the procedures described by, Kung et al., Am J. Respir. Cell Mol. Biol. 13:360-365, (1995) and Schneider et al., Am J. Respir. Cell Mol. Biol. 20:448-457, (1999), which are each incorporated by reference in their entirety. Female Black/6 mice (8-12 weeks of age) were sensitized on day 1 by an intraperitoneal injection (i.p.) of 0.2 mL ova/alum mixture containing 20 μg of ova (Grade 4, Sigma) and 2 mg inject Alum (Pierce). A booster injection was administered on day 14. Mice are challenged on days 28 and 29 with aerosolized 1% ova (in 0.9% saline) for 20 minutes. Mice are euthanized and bronchaveolar lavage samples (3 mL) are collected on day 30, 48 hours post first challenge. Eosinophils were quantified by a FACs/FITC staining method. Compounds of this invention were formulated into a 0.5% carboxymethylcellulose and 2% Tween80 suspension and administered orally to mice which had been sensitized to the allergen, ovalbumin. Compounds which inhibited allergen-induced leucocyte accumulation in the airways of actively sensitized C57BL/6 mice were considered to be active in this model.

Sheep Asthma Model

Following the procedures described by Abraham et al, J. Clin, Invest, 93:776-787 (1994) and Abraham et al, Am J. Respir Crit Care Med 156:696-703 (1997), both of which are incorporated by reference in their entirety. Compounds of this invention have been evaluated by intravenous (saline aqueous solution), oral (2% Tween80, 0.5% carboxymethylcellulose), and aerosol administration to sheep which are hypersensitive to Ascaris suum antigen. Compounds which decrease the early antigen-induced bronchial response and/or block the late-phase airway response, e.g. have a protective effect against antigen-induced late responses and airway hyper-responsiveness (“AHR”), are considered to be active in this model.

Allergic sheep which are shown to develop both early and late bronchial responses to inhaled Ascaris suum antigen were used to study the airway effects of the candidate compounds. Following topical anesthesia of the nasal passages with 2% lidocaine, a balloon catheter was advanced through one nostril into the lower esophagus. The animals were then incubated with a cuffed endotracheal tube through the other nostril with a flexible fiberoptic bronchoscope as a guide.

Pleural pressure was estimated according to Abraham (1994). Aerosols (see formulation below) were generated using a disposable medical nebulizer that provided an aerosol with a mass median aerodynamic diameter of 3.2 μm as determined with an Andersen cascade impactor. The nebulizer was connected to a dosimeter system consisting of a solenoid valve and a source of compressed air (20 psi). The output of the nebulizer was directed into a plastic T-piece, one end of which was connected to the inspiratory port of a piston respirator. The solenoid valve was activated for 1 second at the beginning of the inspiratory cycle of the respirator. Aerosols were delivered at VT of 500 mL and a rate of 20 breaths/minute. A 0.5% sodium bicarbonate solution only was used as a control.

To assess bronchial responsiveness, cumulative concentration-response curves to carbachol was generated according to Abraham (1994). Bronchial biopsies were taken prior to and following the initiation of treatment and 24 hours after antigen challenge. Bronchial biopsies were preformed according to Abraham (1994).

An in vitro adhesion study of alveolar macrophages were also performed according to Abraham (1994), and a percentage of adherent cells calculated.

Aerosol Formulation

A solution of the candidate compound in 0.5% sodium bicarbonate/saline (w/v) at a concentration of 30.0 mg/mL is prepared using the following procedure:

A. Preparation of 0.5% Sodium Bicarbonate/
Saline Stock Solution: 100.0 mL

| Ingredient | Gram/100.0 mL | Final Concentration |
|---|---|---|
| Sodium Bicarbonate | 0.5 g | 0.5% |
| Saline | q.s. ad 100.0 mL | q.s. ad 100% |

Procedure:
1. Add 0.5 g sodium bicarbonate into a 100 mL volumetric flask.
2. Add approximately 90.0 mL saline and sonicate until dissolved.
3. Q.S. to 100.0 mL with saline and mix thoroughly.

B. Preparation of 30.0 mg/mL Candidate Compound: 10.0 mL

| Ingredient | Gram/10.0 mL | Final Concentration |
|---|---|---|
| Candidate | 0.300 g | 30.0 mg/mL |
| Compound | | |
| 0.5% Sodium | q.s. ad 10.0 mL | q.s ad 100% |
| Bicarbonate/Saline | | |
| Stock Solution | | |

Procedure:
1. Add 0.300 g of the candidate compound into a 10.0 mL
volumetric flask.
2. Add approximately 9.7 mL of 0.5% sodium bicarbonate/saline stock
solution.
3. Sonicate until the candidate compound is completely
dissolved.
4. Q.S. to 10.0 mL with 0.5% sodium bicarbonate/saline stock
solution and mix thoroughly.

### EXAMPLE E

### 10-Day Toxicity Study on C57B6 Mice

A 10-day study was conducted to evaluate the toxicity of Compounds of the present invention to female C57B6 mice. The compound was administered by gavage at five dose levels, 0 (vehicle control), 10, 30, 100, 300 and 1000 mg/kg (mpk), with five mice in each dose level. The dose volume for all levels was 10 mL/kg. Dose solutions or suspensions were prepared in 2% Tween 80 in 0.5% carboxymethyl cellulose (CMC) and new dose solutions or suspensions were prepared every two-three days. In-life observations included body weights (study day 1, 2, 3, 5, 7, 8 and 11), daily cageside clinical observations (1-2/day) and periodic (study day −1, 2 and 9) functional observation battery.

At termination, blood samples were collected by cardiac puncture for clinical pathology (hematology and clinical chemistry) and drug levels. The EDTA blood samples were analyzed for total white blood cell count, red blood cell count, hemoglobin, hematocrit, erythrocyte indices (MCV, MCH, MCHC), platelets and a WBC five part differential (neutrophil, lymphocytes, monocytes, eosinophils and basophils). Heparinized plasma samples were analyzed for alanine transaminase, aspartate transaminase, alkaline phosphatase, total bilirubin, albumin, protein, calcium, glucose, urea nitrogen, creatinine, cholesterol and triglycerides.

After blood collection, the carcass was necropsied and organs (liver, spleen, kidneys, heart and thymus) were weighed. Tissue samples; brain, salivary glands, thymus, heart, lung, liver, kidney, adrenal spleen, stomach, duodenum, ileum, colon and uterus/ovary, were collected and formalin fixed. Tissues from the vehicle control and 300 and 1000 mpk group animals were processed to H & E stained glass slides and evaluated for histopathological lesions.

Body weight changes, absolute and relative organ weights and clinical pathology results were analyzed for statistical significant differences compared to the vehicle controls by Dunnet's multiple comparison test using Prism software. The functional observation battery results were analyzed for differences using the Dunnet's, Fisher's exact tests and dose trend effects by the Cochran-Mantel-Haenszel correlation test using SAS software.

Using a conventional oral formulation, compounds of this invention would be active in this model.

### EXAMPLE F

### In Vivo Evaluation

The standard multiple sclerosis model, Experimental Autoimmune (or Allergic) Encephalomyelitis (“EAE”), was used to determine the effect of candidate compounds to reduce motor impairment in rats or guinea pigs. Reduction in motor impairment is based on blocking adhesion between leukocytes and the endothelium and correlates with anti-inflammatory activity in the candidate compound. This model has been previously described by Keszthelyi et al., Neurology, 1996, 47:1053-1059, and measures the delay of onset of disease.

Brains and spinal cords of adult Hartley guinea pigs were homogenized in an equal volume of phosphate-buffered saline. An equal volume of Freund's complete adjuvant (100 mg mycobacterium tuberculosis plus 10 ml Freund's incomplete adjuvant) was added to the homogenate. The mixture was emulsified by circulating it repeatedly through a 20 ml syringe with a peristaltic pump for about 20 minutes.

Female Lewis rats (2-3 months old, 170-220 g) or Hartley guinea pigs (20 day old, 180-200 g) were anesthetized with isoflurane and three injections of the emulsion, 0.1 ml each, were made in each flank. Motor impairment onset is seen in approximately 9 days.

Candidate compound treatment began on Day 8, just before onset of symptoms. Compounds were administered subcutaneously (“SC”), orally (“PO”) or intraperitoneally (“IP”). Doses were given in a range of 10 mg/kg to 200 mg/kg, bid, for five days, with typical dosing of 10 to 100 mg/kg SC, 10 to 50 mg/kg PO, and 10 to 100 mg/kg IP.

Antibody GG5/3 against α₄ μl integrin (Keszthelyi et al., Neurology, 1996, 47:1053-1059), which delays the onset of symptoms, was used as a positive control and was injected subcutaneously at 3 mg/kg on Day 8 and 11.

Body weight and motor impairment were measured daily. Motor impairment was rated with the following clinical score:

| | |
|---|---|
| 0 | no change |
| 1 | tail weakness or paralysis |
| 2 | hindlimb weakness |
| 3 | hindlimb paralysis |
| 4 | moribund or dead |

A candidate compound was considered active if it delayed the onset of symptoms, e.g., produced clinical scores no greater than 2 or slowed body weight loss as compared to the control.

### 11. TREATMENT EXAMPLES

The following examples are put forth so as to provide those of ordinary skill in the art with a complete disclosure and description of how to make and use the present invention, and is not intended to limit the scope of what the inventors regard as their invention nor is it intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Construction of Humanized 21.6 Antibody

Chimeric light and heavy chains were constructed by linking the PCR-cloned cDNAs of mouse 21.6 V_{L} and V_{H} regions to human constant regions. The 5′- and 3′-ends of the mouse cDNA sequences were modified using specially designed PCR primers. The 5′-end PCR-primers (Table 14), which hybridize to the DNA sequences coding for the beginnings of the leader sequences, were designed to create the DNA sequences essential for efficient translation (Kozak, J. Mol. Biol. 196: 947-950 (1987)), and to create a HindIII restriction sites for cloning into an expression vector. The 3′-end primers, which hybridize to the DNA sequences coding for the ends of J regions, were designed to create the DNA sequences essential for splicing to the constant regions, and to create a BamHI site for cloning into an expression vector. The products of PCR amplification were digested with HindIII and BamHI, cloned into a pUC 19 vector, and sequenced to confirm that no errors had occurred during PCR amplification. The adapted mouse 21.6 variable regions were then subcloned into mammalian cells expression vectors containing either the human kappa or gamma-1 constant regions.

TABLE 14
PCR Primers for the Construction of Chimeric 21.6 Antibody

```
A. Light Chain Variable Region
1. Primer for reconstruction of the 5′-end (37-mer)
5′C AGA AAG CTT GCC GCC ACC ATG AGA CCG TCT ATT CAG 3′
                HindIII Kozak M R P S I Q
                                Consensus
                                Sequence
2. Primer for reconstruction of the 3′-end (35-mer)
5′CC GAG GAT CCA CTC ACG TTT GAT TTC CAG CTT GGT 3′
                  BamHI Splice donor site
B. Heavy chain variable region
1. Primer for reconstruction of the 5′-end (37-mer)
5′C AGA AAG CTT GCC GCC ACC ATG AAA TGC AGC TGG GTC 3′
                HindIII Kozak M K C S W V
                                Consensus
                                Sequence
2. Primer for reconstruction of the 3′-end (33-mer)
5′CC GAG GAT CCA CTC ACC TGA GGA GAC GGT GAC T 3′
                  BamHI Splice donor site
```

Modeling the Structure of the Mouse 21.6 Variable Regions. A molecular model of the V_{L} and V_{H} regions of mouse 21.6 antibody was built. The model was built on a Silicon Graphics IRIS 4D workstation running under the UNIX operating system and using the molecular modeling package QUANTA (Polygen Corp., USA). The structure of the FRs of mouse 21.6 V_{L} region was based on the solved structure of human Bence-Jones immunoglobulin RE1 (Epp et al., Biochemistry 14: 4943-4952 (1975)). The structure of the FRs of mouse 21.6 V_{H} region was based on the solved structure of mouse antibody Gloop2. Identical residues in the FRs were retained; non-identical residues were substituted using the facilities within QUANTA. CDR1 and CDR2 of mouse 21.6 V_{L} region were identified as belonging to canonical structure groups 2 and 1, respectively (Chothia et al., J. Mol Biol. 196: 901-917 (1987)). Since CDR1 and CDR2 of RE1 belong to the same canonical groups, CDR1 and CDR2 of mouse 21.6, V_{L} region were modeled on the structures of CDR1 and CDR2 of RE1. CDR3 of mouse 21.6 V_{L} region did not appear to correspond to any of the canonical structure groups for CDR3s of V_{L} regions. A database search revealed, however, that CDR3 in mouse 21.6 V_{L} region was similar to CDR3 in mouse HyHEL-5 V_{L} region (Sheriff et al., Proc. Natl. Acad. Sci. USA 84: 8075-8079 (1987)). Thus, the CDR3 of mouse 21.6 V_{L} region was modeled on the structure of CDR3 in mouse HyHEL-5 V_{L} region. CDR1 and CDR2 of mouse 21.6 V_{H} region were identified as belonging to canonical structure groups 1 and 2, respectively. CDR1 of mouse 21.6 V_{H} region was modeled on CDR1 of Gloop2 V_{H} region, which closely resembles members of canonical group 1 for CDR1s of V_{H} regions. CDR2 of mouse 21.6 V_{H} region was modeled on CDR2 of mouse HyHEL-5 (Sheriff et al., supra), which is also a member of canonical group 2 for CDR2 for V_{H} regions. For CDR3s of V_{H} regions, there are no canonical structures. However, CDR3 in mouse 21.6 V_{H} region was similar to CDR3 in mouse R19.9 V_{H} region (Lascombe et al., Proc. Natl. Acad. Sci. USA 86: 607-611 (1989)) and was modeled on this CDR3 by removing an extra serine residue present at the apex of the CDR3 loop of mouse R19.9 V_{H} region and annealing and refining the gap. The model was finally subjected to steepest descents and conjugate gradients energy minimization using the CHARMM potential (Brooks et al., J. Comp. Chem. 4: 187-217 (1983)), as implemented in QUANTA in order to relieve unfavorable atomic contacts and to optimize van der Waals and electrostatic interactions.

Design of Reshaped Human 21.6 Variable Regions —Selection of Homologous Human Antibodies for Framework Sequence. Human variable regions whose FRs showed a high percent identity to those of mouse 21.6 were identified by comparison of amino acid sequences. Tables 16 and 17 compare the mouse 21.6 variable regions to all known mouse variable regions and then to all known human variable regions. The mouse 21.6 V_{L} region was identified as belonging to mouse kappa V_{L} region subgroup 5 as defined by Kabat. Individual mouse kappa V_{L} regions were identified that had as much as 93.4% identity to the mouse 21.6 kappa V_{L} region (38C13V′CL and PC613′CL). Mouse 21.6 V_{L} region was most similar to human kappa V_{L} regions of subgroup 1, as defined by Kabat. Individual human kappa V_{L} regions were identified that had as much as 72.4% identity to the mouse 21.6 kappa V_{L} region. The framework regions (FRs) from one of the most similar human variable regions, RE1, were used in the design of reshaped human 21.6 V_{L} region. Mouse 21.6 V_{H} region was identified as belonging to mouse V_{H} region subgroup 2c as defined by Kabat. Individual mouse heavy chain variable regions were identified that have as much as 93.3% identity to the mouse 21.6 V_{H} region (17.20.25′CL and 87.92.6′CL). Mouse 21.6 V_{H} region was most similar to human V_{H} regions of subgroup 1 as defined by Kabat et al., supra. Individual human V_{H} regions were identified that had as much as 64.7% identity to the mouse 21.6 V_{H} region. The FRs from one of the most similar human variable regions, 21/28′CL, was used in the design of reshaped human 21.6 V_{H} region.

Substitution of Amino Acids in Framework Regions.

(A) Light Chain. The next step in the design process for the reshaped human 21.6 V_{L} region was to join the CDRs from mouse 21.6 V_{L} region to the FRs from human RE1 (Palm et al., Physiol. Chem. 356: 167-191 (1975). In the first version of reshaped human 21.6 V_{L} region (La), seven changes were made in the human FRs. At positions 104, 105, and 107 in FR4, amino acids from RE1 were substituted with more typical human J region amino acids from another human kappa light chain (Riechmann et al., Nature 332:323-327 (1988)).

At position 45 in FR2, the lysine normally present in RE1 was changed to an arginine as found at that position in mouse 21.6 V_{L} region. The amino acid residue at this position was thought to be important in the supporting the CDR2 loop of the mouse 21.6 V_{L} region.

At position 49 in FR2, the tyrosine normally present in RE1 was changed to a histidine as found at that position in mouse 21.6 V_{L} region. The histidine at this position in mouse 21.6 V_{L} region was observed in the model to be located in the middle of the binding site and could possibly make direct contact with antigen during antibody-antigen binding.

At position 58 in FR3, the valine normally present in RE1 was changed to an isoleucine as found at that position in mouse 21.6 V_{L} region. The amino acid residue at this position was thought to be important in the supporting the CDR2 loop of the mouse 21.6 V_{L} region.

At position 69 in FR3, the threonine normally present in RE1 was changed to an arginine as found at that position in mouse 21.6 V_{L} region. The arginine at this position in mouse 21.6 V_{L} region was observed in the model to be located adjacent to the CDR1 loop of mouse 21.6 V_{L} region and could possibly make direct contact with the antigen during antibody-antigen binding.

A second version of reshaped human 21.6 V_{L} region (termed Lb) was designed containing the same substitutions as above except that no change was made at position 49 in FR2 of RE1.

(B) Heavy Chain. The next step in the design process for the reshaped human 21.6 V_{H} region was to join the CDRs from mouse 21.6 V_{H} region to the FRs from 21/28′CL (Dersimonian et al., J. Immunol. 139: 2496-2501 (1987)). In the first version of reshaped human 21.6 V region (Ha), five changes were made in the human framework regions. The five changes in the human FRs were at positions 27, 28, 29, 30, and 71.

At positions 27, 28, 29, and 30 in FR1, the amino acids present in human 21/28′CL were changed to the amino acids found at those positions in mouse 21.6 V_{H} region. Although these positions are designated as being within FR1 (Kabat et al., supra), positions 26 to 30 are part of the structural loop that forms the CDR1 loop of the V_{H} region. It is likely, therefore, that the amino acids at these positions are directly involved in binding to antigen. Indeed, positions 27 to 30 are part of the canonical structure for CDR1 of the V_{H} region as defined by Chothia et al., supra.

At position 71 in FR3, the arginine present in human 21/28′CL was changed to a alanine as found at that position in mouse 21.6 V_{H} region. Position 71 is part of the canonical structure for CDR2 of the V_{H} region as defined by Chothia et al., supra. From the model of the mouse 21.6 variable regions, it appears that the alanine at position 71 is important in supporting the CDR2 loop of the V_{H} region. A substitution of an arginine for an alanine at this position would very probably disrupt the placing of the CDR2 loop.

A second version (Hb) of reshaped human 21.6 V_{H} region contains the five changes described above for version Ha were made plus one additional change in FR2.

At position 44 in FR2, the arginine present in human 21/28′CL was changed to a glycine as found at that position in mouse 21.6 V_{H} region. Based on published information on the packing of V_{L}-V_{H} regions and on the model of the mouse 21.6 variable regions, it was thought that the amino acid residue at position 44 might be important in the packing of the V_{L}-V_{H} regions.

Reshaped human 21.6 V region version Hc was designed to make the CDR3 loop look more similar to human VCAM-1. Both mouse 21.6 antibody and human VCAM-1 bind to the α₄β₁ integrin. The CDR3 loop of the V_{H} region of antibodies is the most diverse of the six CDR loops and is generally the most important single component of the antibody in antibody-antigen interactions (Chothia et al., supra; Hoogenboom & Winter, J. Mol. Biol. 227: 381-388 (1992); Barbas et al., Proc. Natl. Acad. Sci. USA 89: 4457-4461 (1992)). Some sequence similarity was identified between the CDR3 of mouse 21.6 V_{H} region and amino acids 86 to 94 of human VCAM-1, particularly, between the YGN (Tyrosine-Glycine-Asparagine) sequence in the CDR3 loop and the FGN (i.e., Phenylalanine-Glycine-Asparagine) sequence in VCAM-1. These sequences are thought to be related to the RGD (i.e., Arginine-Glycine-Aspartic acid) sequences important in various cell adhesion events (Main et al., Cell 71: 671-678 (1992)). Therefore, at position 98 in CDR3, the tyrosine present in mouse 21.6 V_{H} region was changed to a phenylalanine as found in the sequence of human VCAM-1.

Possible substitution at position 36 in FR1 was also considered. The mouse 21.6 V_{H} chain contains an unusual cysteine residue at position 36 in FR2. This position in FR2 is usually a tryptophan in related mouse and human sequences. Although cysteine residues are often important for conformation of an antibody, the model of the mouse 21.6 variable regions did not indicate that this cysteine residue was involved either directly or indirectly with antigen binding so the tryptophan present in FR2 of human 21/28′CL V_{H} region was left unsubstituted in all three versions of humanized 21.6 antibody.

Construction of Reshaped Human 21.6 Antibodies. The first version of reshaped human 21.6 V_{L} region (resh21.6VLa) was constructed from overlapping PCR fragments essentially as described by Daugherty et al., Nucleic Acids Res. 19: 2471-2476 (1991). The mouse 21.6 V_{L} region, adapted as described supra and inserted into pUC19, was used as a template. Four pairs of primers, APCR1-vla1, vla2-vla3, vla4-vla5, and vla6-vla7 were synthesized. Adjacent pairs overlapped by at least 21 bases. The APCR1 primer is complementary to the pUC 19 vector. The appropriate primer pairs (0.2 μmoles) were combined with 10 ng of template DNA, and 1 unit of AmpliTaq DNA polymerase (Perkin Elmer Cetus) in 50 μl of PCR buffer containing 10 mM Tris-HCl (pH 8.3), 50 mM KCl, 200 μM dNTPs, and 1.5 mM MgCl₂. Each reaction was carried out for 25 cycles. After an initial melt at 94° C. for 5 min, the reactions were cycled at 94° C. for 1 min, 55° C. for 1 min, and 72° C. for 2 min, and finally incubated at 72° C. for a further 10 min. The ramp time between the primer-annealing and extension steps was 2.5 min. The products of the four reactions (A, B, C, and D) from the first round of PCR reactions were phenol-extracted and ethanol-precipitated.

TABLE 15
PCR primers for the construction of reshaped human
21.6 variable regions

A. Light chain variable region
1. Primers for the synthesis of version “a”
21.6VLa1 (39-mer):
5′ GAT GGT GAC TCT ATC TCC TAC AGA TGC AGA CAG TGA
GGA 3′
21.6VLa2 (32-mer):
5′ CTG TAG GAG ATA GAG TCA CCA TCA CTT GCA AG 3′
21.6VLa3 (39-mer):
5′ AGG AGC TTT TCC AGG TGT CTG TTG GTA CCA AGC CAT
ATA 3′
21.6VLa4 (41 -mer):
5′ ACC AAC AGA CAC CTG GAA AAG CTC CTA GGC TGC TCA
TAC AT 3′
21.6VLa5 (40-mer):
5′ GCA GGC TGC TGA TGG TGA AAG TAT AAT CTC TCC CAG
ACC C 3′
21.6VLa6 (42-mer):
5′ ACT TTC ACC ATC AGC AGC CTG CAG CCT GAA GAT ATT
GCA ACT 3′
21.6VLa7 (59-mer):
5′ CCG AGG ATC CAC TCA CGT TTG ATT TCC ACC TTG GTG
CCT TGA CCG AAC GTC CAC AGA TT 3′
2. Primers for the synthesis of version “b”
21.6VLb1 (33-mer): changes H-49 to Y-49
5′ GGA AAA GCT CCT AGG CTG CTC ATA TAT TAC ACA 3′
21.6VLb2 (38-mer): changes ACC-101 to ACA-101 to
destroy an StyI site
5′ CCG AGG ATC CAC TCA CGT TTG ATT TCC ACC TTT GTG
CC 3′
B. Heavy chain variable region
1. Primers for the synthesis of version “a”
21.6VHa1 (51-mer):
5′ AAC CCA GTG TAT ATA GGT GTC TTT AAT GTT GAA ACC
GCT AGC TTT ACA GCT 3′
21.6VHa2 (67-mer):
5′ AAA GAC ACC TAT ATA CAC TGG GTT AGA CAG GCC CCT
GGC CAA AGG CTG GAG TGG ATG GGA AGG ATT G 3′
21.6VHa3 (26-mer):
5′ GAC CCG GCC CTG GAA CTT CGG GTC AT 3′
21.6VHa4 (66-mer):
5′ GAC CCG AAG TTC CAG GGC CGG GTC ACC ATC ACC GCA
GAC ACC TCT GCC AGC ACC GCC TAC ATG GAA 3′
21.6VHa5 (64-mer):
5′ CCA TAG CAT AGA CCC CGT AGT TAC CAT AAT ATC CCT
CTC TGG CGC AGT AGT AGA CTG CAG TGT G 3′
21.6VHa6 (63-mer):
5′ GGT AAC TAC GGG GTC TAT GCT ATG GAC TAC TGG GGT
CAA GGA ACC CTT GTC ACC GTC TCC TCA 3′
2. Primer for the synthesis of version “b”
21.6VHb (37-mer): changes R-44 to G-44
5′ CCA GGG CCG GGTCAC CAT CAC CAG AGA CAC CTC
TGC C 3′
3. Primer for the synthesis of version “c”
21.6VHc (27-mer): changes Y-98 to F-98
5′ CAG GCC CCT GGC CAA GGG CTG GAG TGG 3′
C. Both light and heavy chain variable regions
Primers hybridizing to the flanking pUC19 vectcr
DNA APCR1 (17-mer, sense primer)
5′ TAC GCA AAC CGC CTC TC 3′
APCR4 (18-mer, anti-sense primer)
5′ GAG TGC ACC ATA TGC GGT 3′

PCR products A and B, and C and D were joined in a second round of PCR reactions. PCR products A and B, and C and D, (50 ng of each) were added to 50 μl PCR reactions (as described supra) and amplified through 20 cycles as described above, except that the annealing temperature was raised to 60° C. The products of these reactions were termed E and F. The pairs of PCR primers used were APCR1-vla3 and vla4-vla7, respectively. PCR products E and F were phenol-extracted and ethanol-precipitated and then assembled in a third round of PCR reactions by their own complementarity in a two step-PCR reaction similar to that described above using APCR1 and vla7 as the terminal primers. The fully assembled fragment representing the entire reshaped human 21.6 V_{L} region including a leader sequence was digested with HindIII and BamHI and cloned into pUC 19 for sequencing. A clone having the correct sequence was designated resh21.6VLa.

The second version of a reshaped human 21.6 V_{L} region (Lb) was constructed using PCR primers to make minor modifications in the first version of reshaped human 21.6 V_{L} region (La) by the method of Kamman et al., Nucl. Acids Res. 17: 5404 (1989). Two sets of primers were synthesized. Each PCR reaction was essentially carried out under the same conditions as described above. In a first PCR reaction, mutagenic primer 21.6VLb2 was used to destroy a StyI site (Thr-ACC-97 to Thr-ACA-97) to yield resh21.6VLa2. Then, in a second PCR reaction, mutagenic primer 21.6VLb1 (His-49 to Tyr-49) was used with pUC-resh21.6VLa2 as template DNA. The PCR product was cut with StyI and BamHI and subcloned into pUC-resh21.6VLa2, cleaved with the same restriction enzymes. A clone with the correct sequence was designated pUC-resh21.6VLb.

Version “a” of a reshaped human 21.6 V_{H} region was constructed using the same PCR methods as described for the construction of version “a” of reshaped human 21.6 V_{L} region. The HindIII-BamHI DNA fragments coding for version “g” of reshaped human 425 V_{H} region (Kettleborough et al., supra) and version “b” of reshaped human AUK12-20 V_{H} region were subcloned into pUC19 vectors yielding pUC-resh425g and pUC-reshAUK12-20b, respectively. (Version “b” of AUK12-20, was derived by PCR mutagenesis of a fragment V_{H} a425 described by Kettleborough et al., supra, and encodes the amino acid sequence: QVQLVQSGAEVKKPGASVKVSCKASGYSFT SYYIH WVRQAPGQGLEWVG YIDPFNGGTSYNQKFKG KVTMTVDTSTNTAYMELSSLRSEDTAVYYCAR GGN-RFAY WGQGTLVTVSS (spaces separate FR and CDR regions)).

Plasmid pUC-resh425g and pUC-reshAUK12-20b, as well as the pUC vector containing the mouse 21.6 V_{H} region as modified for use in the construction of the chimeric 21.6 heavy chain (pUC-chim21.6V_{H}), were used as template DNAs in the subsequent PCR reactions. PCR primers were designed and synthesized for the construction of version “a” of reshaped human 21.6 V_{H} region. PCR product A was obtained using pUC-reshAUK12-20b as DNA template and APCR1-vha1 as the PCR primer pair. PCR products B and D were obtained using pUC-chim21.6V_{H} as DNA template and vha2-vha3 and vha6-APCR4 as PCR primer pairs, respectively. Finally, PCR product C was obtained using pUC-resh425g as DNA template and vla4-vla5 as the PCR primer pair. The final PCR product was subcloned into pUC 19 as a HindIII-BamHI fragment for DNA sequencing. A clone with the correct DNA sequence was designated pUC-resh21.6VHa.

The remaining versions of reshaped human 21.6 V_{H} region were constructed essentially as described above for the construction of version “b” of reshaped human 21.6 V_{L} region. Two sets of primers were synthesized. For the second (Hb) and third (Hc) versions, mutagenic primers 21.6VHb (Arg-44 to Gly-44) and 21.6VHc (Tyr-98 to Phe-98), respectively, were used in PCR reactions with pUC-resh21.6VHa as the template DNA. The PCR products VHb and VHc were cut with restriction enzymes and subcloned into pUC vector pUC-resh21.6VHa as MscI-BamHI and PstI-BamHI fragments, respectively, to yield pUC-resh21.6VHb and pUC-resh21.6VHc.

The first version of a reshaped human 21.6 V_{H} region (Ha) was constructed in a similar manner to that used for the construction of the first version of reshaped human 21.6 V_{L} region (La). In this case, however, PCR primers were used with three different template DNAs, mouse 21.6 V_{H} region as already adapted for expression of chimeric 21.6 heavy chain, humanized 425 V_{H} region version “g” (Kettleborough et al., supra), and humanized AUK12-20 version “b” V_{H} region. The second and third versions of a humanized 21.6 V_{H} region (Hb and Hc) were constructed using PCR primers to make minor modifications in the first version of humanized 21.6 V_{H} region (Ha).

TABLE 16
Alignment of amino acid sequences leading to the design
of reshaped human 21.6 light chain variable regions.

| | | FR or | mouse | mouse | human | human | RH V_{L} | |
|---|---|---|---|---|---|---|---|---|
| Kabat | # | CDR | 21.6 | kappa 5 | kappa 1 | RE1 | 21.6 | Comment |
| 1 | 1 | FR1 | D | D | D | D | D | |
| 2 | 2 | \| | I | I | I | I | | |
| 3 | 3 | \| | Q | Q | Q | Q | Q | |
| 4 | 4 | \| | M | M | M | M | M | |
| 5 | 5 | \| | T | T | T | T | T | |
| 6 | 6 | \| | Q | Q | Q | Q | Q | |
| 7 | 7 | \| | S | S | S | S | S | |
| 8 | 8 | \| | P | P | P | P | P | |
| 9 | 9 | \| | S | S | S | S | S | |
| 10 | 10 | \| | S | S | S | S | S | |
| 11 | 11 | \| | L | L | L | L | L | |
| 12 | 12 | \| | S | S | S | S | S | |
| 13 | 13 | \| | A | A | A | A | A | |
| 14 | 14 | \| | S | S | S | S | S | |
| 15 | 15 | \| | L | L | V | V | V | |
| 16 | 16 | \| | G | G | G | G | G | |
| 17 | 17 | \| | G | D | D | D | D | |
| 18 | 18 | \| | K | R | R | R | R | |
| 19 | 19 | \| | V | V | V | V | V | |
| 20 | 20 | \| | T | T | T | T | T | |
| 21 | 21 | \| | I | I | I | I | I | |
| 22 | 22 | \| | T | T | T | T | T | |
| 23 | 23 | FR1 | C | C | C | C | C | |
| 24 | 24 | CDR1 | K | R | R | Q | K | |
| 25 | 25 | \| | T | A | A | A | T* | |
| 26 | 26 | \| | S | S | S | S | S | |
| 27 | 27 | \| | Q | Q | Q | Q | Q* | |
| 27A | | \| | — | D | S | — | — | |
| 27B | | \| | — | — | L | — | — | |
| 27C | | \| | — | — | V | — | — | |
| 27D | | \| | — | — | X | — | | |
| 27E | | \| | — | — | X | — | — | |
| 27F | | \| | — | — | — | — | — | |
| 28 | 28 | \| | D | D | S | D | D* | |
| 29 | 29 | \| | I | I | I | I | I* | |
| 30 | 30 | \| | N | S | S | I | N* | |
| 31 | 31 | \| | K | N | N | K | K* | |
| 32 | 32 | \| | Y | Y | Y | Y | Y* | |
| 33 | 33 | \| | M | L | L | L | M* | |
| 34 | 34 | CDR1 | A | N | A | N | A | |
| 35 | 35 | FR2 | W | W | W | W | W | |
| 36 | 36 | \| | Y | Y | Y | Y | Y | |
| 37 | 37 | \| | Q | Q | Q | Q | Q | |
| 38 | 38 | \| | H | Q | Q | Q | Q | |
| 39 | 39 | \| | K | K | K | T | T | K in CAMPATH-1H |
| 40 | 40 | \| | P | P | P | P | P | |
| 41 | 41 | \| | G | G | G | G | G | |
| 42 | 42 | \| | K | G | K | K | K | |
| 43 | 43 | \| | R | S | A | A | A | consider R in other |
| | | | | | | | | versions |
| 44 | 44 | \| | P | P | P | P | P | |
| 45 | 45 | \| | R | K | K | K | R | supports L2 loop, |
| | | | | | | | | consider K in other |
| | | | | | | | | versions |
| 46 | 46 | \| | L | L | L | L | L | |
| 47 | 47 | \| | L | L | L | L | L | |
| 48 | 48 | \| | I | I | I | I | I* | |
| 49 | 49 | \| | H | Y | Y | Y | H | in middle of binding |
| | | | | | | | | site, potential to |
| | | | | | | | | interact with antigen, |
| | | | | | | | | consider Y in other |
| | | | | | | | | versions |
| 50 | 50 | CDR2 | Y | Y | A | E | Y* | |
| 51 | 51 | \| | T | A | A | A | T* | |
| 52 | 52 | \| | S | S | S | S | S* | |
| 53 | 53 | \| | A | R | S | N | A | |
| 54 | 54 | \| | L | L | L | L | L | |
| 55 | 55 | \| | Q | H | E | Q | Q | |
| 56 | 56 | CDR2 | P | S | S | A | P | |
| 57 | 57 | FR3 | G | G | G | G | G | |
| 58 | 58 | \| | I | V | V | V | I | maybe supporting L2, |
| | | | | | | | | consider V in other |
| | | | | | | | | versions |
| 59 | 59 | \| | P | P | P | P | P | |
| 60 | 60 | \| | S | S | S | S | S | |
| 61 | 61 | \| | R | R | R | R | R | |
| 62 | 62 | \| | F | F | F | F | F | |
| 63 | 63 | \| | S | S | S | S | S | |
| 64 | 64 | \| | G | G | G | G | G* | |
| 65 | 65 | \| | S | S | S | S | S | |
| 66 | 66 | \| | G | G | G | G | G | |
| 67 | 67 | \| | S | S | S | S | S | |
| 68 | 68 | \| | G | G | G | G | G | |
| 69 | 69 | \| | R | T | T | T | R | adjacent to L1, on the |
| | | | | | | | | surface near the |
| | | | | | | | | binding site |
| 70 | 70 | \| | D | D | D | D | D | |
| 71 | 71 | \| | Y | Y | F | Y | Y* | F in CAMPATH-1H |
| 72 | 72 | \| | S | S | T | T | T | |
| 73 | 73 | \| | F | L | L | P | F | |
| 74 | 74 | \| | N | T | T | T | T | |
| 75 | 75 | \| | I | I | I | I | I | |
| 76 | 76 | \| | S | S | S | S | S | |
| 77 | 77 | \| | N | N | S | S | S | |
| 78 | 78 | \| | L | L | L | L | L | |
| 79 | 79 | \| | E | E | Q | Q | Q | |
| 80 | 80 | \| | P | Q | P | P | P | |
| 81 | 81 | \| | E | E | E | E | E | |
| 82 | 82 | \| | D | D | D | D | D | |
| 83 | 83 | \| | I | I | F | I | I | |
| 84 | 84 | \| | A | A | A | A | A | |
| 85 | 85 | \| | T | T | T | T | T | |
| 86 | 86 | \| | Y | Y | Y | Y | Y | |
| 87 | 87 | \| | Y | F | Y | Y | Y | |
| 88 | 88 | FR3 | C | C | C | C | C | |
| 89 | 89 | CDR3 | L | Q | Q | Q | L | |
| 90 | 90 | \| | Q | Q | Q | Q | Q* | |
| 91 | 91 | \| | Y | G | Y | Y | Y* | |
| 92 | 92 | \| | D | N | N | Q | D* | |
| 93 | 93 | \| | N | T | S | S | N* | |
| 94 | 94 | \| | L | L | L | L | L* | |
| 95 | 95 | \| | — | P | P | P | — | |
| 95A | | \| | — | P | E | — | — | |
| 95B | | \| | — | — | — | — | — | |
| 95C | | \| | — | — | — | — | — | |
| 95D | | \| | — | — | — | — | — | |
| 95E | | \| | — | — | — | — | — | |
| 95F | | \| | — | — | — | — | — | |
| 96 | 95 | \| | W | R | W | Y | W* | |
| 97 | 96 | CDR3 | T | T | T | T | T | |
| 98 | 97 | FR4 | F | F | F | F | F | |
| 99 | 98 | \| | G | G | G | G | G | |
| 100 | 99 | \| | G | G | Q | Q | Q | |
| 101 | 100 | \| | G | G | G | G | G | |
| 102 | 101 | \| | T | T | T | T | T | |
| 103 | 102 | \| | K | K | K | K | K | |
| 104 | 103 | \| | L | L | V | L | V | as in CAMPATH-1H |
| 105 | 104 | \| | E | E | E | Q | E | as in CAMPATH-1H |
| 106 | 105 | \| | I | I | I | I | I | |
| 106A | | \| | — | — | — | — | — | |
| 107 | 106 | FR4 | K | K | K | T | K | as in CAMPATH-1H |

Legend:
(Kabat) numbering according to Kabat et al., supra;
(#) sequential numbering as used in the molecular modeling;
(mouse 21.6) amino acid sequence of the V_{L} region from mouse 21.6 antibody;
(mouse kappa 5) consensus sequence of mouse kappa V_{L} regions from subgroup 5 (Kabat et al., supra);
(human kappa 1) consensus sequence of human V_{L} regions from subgroup 1 (Kabat et al., supra);
(human RED amino acid sequence of a human V_{L} region (Palm et al., Physiol. Chem. 356: 167-191 (1975));
(RH V_{L} 21.6) amino acid sequence of version L1 of reshaped human 21.6 V_{L} region;
*residues that are part of the canonical structures for the CDR loops (Chothia et al., supra);
(underlined) residues in the human FRs where the amino acid residue was changed.

TABLE 17
Alignment of amino acid sequences leading to the design of
reshaped human 21.6 heavy chain variable regions.

| | | FR or | mouse | mouse | | human | RH V_{H} | |
|---|---|---|---|---|---|---|---|---|
| Kabat | # | CDR | 21.6 | 2c | human 1 | 21/28′CL | 21.6 | Comment |
| 1 | 1 | FR1 | E | E | Q | Q | Q | |
| 2 | 2 | \| | V | V | V | V | V | |
| 3 | 3 | \| | Q | Q | Q | Q | Q | |
| 4 | 4 | \| | L | L | L | L | L | |
| 5 | 5 | \| | Q | Q | V | V | V | |
| 6 | 6 | \| | Q | Q | Q | Q | Q | |
| 7 | 7 | \| | S | S | S | S | S | |
| 8 | 8 | \| | G | G | G | G | G | |
| 9 | 9 | \| | A | A | A | A | A | |
| 10 | 10 | \| | E | E | E | E | E | |
| 11 | 11 | \| | L | L | V | V | V | |
| 12 | 12 | \| | V | V | K | K | K | |
| 13 | 13 | \| | K | K | K | K | K | |
| 14 | 14 | \| | P | P | P | P | P | |
| 15 | 15 | \| | G | G | G | G | G | |
| 16 | 16 | \| | A | A | A | A | A | |
| 17 | 17 | \| | S | S | S | S | S | |
| 18 | 18 | \| | V | V | V | V | V | |
| 19 | 19 | \| | K | K | K | K | K | |
| 20 | 20 | \| | L | L | V | V | V | |
| 21 | 21 | \| | S | S | S | S | S | |
| 22 | 22 | \| | C | C | C | C | C | |
| 23 | 23 | \| | T | T | K | K | K | |
| 24 | 24 | \| | A | A | A | A | A | |
| 25 | 25 | \| | S | S | S | S | S | |
| 26 | 26 | \| | G | G | G | G | G* | |
| 27 | 27 | \| | F | F | Y | Y | F* | H1 canonical structure, |
| | | | | | | | | consider Y in other |
| | | | | | | | | versions |
| 28 | 28 | \| | N | N | T | T | N* | H1 canonical structure, |
| | | | | | | | | on the surface |
| 29 | 29 | \| | I | I | F | F | I* | H1 canonical structure, |
| | | | | | | | | consider F in other |
| | | | | | | | | versions |
| 30 | 30 | FR1 | K | K | T | T | K* | H1 canonical structure, |
| | | | | | | | | on the surface |
| 31 | 31 | CDR1 | D | D | S | S | D* | |
| 32 | 32 | \| | T | T | Y | Y | T* | |
| 33 | 33 | \| | Y | Y | A | A | Y | |
| 34 | 34 | \| | I | M | I | M | I* | |
| 35 | 35 | \| | H | H | S | H | H | |
| 35A | | \| | — | — | — | — | — | |
| 35B | | CDR1 | — | — | — | — | — | |
| 36 | 36 | \|FR2 | C | W | W | W | W | buried residue, no |
| | | | | | | | | obvious special role for C |
| 37 | 37 | \| | V | V | V | V | V | |
| 38 | 38 | \| | K | K | R | R | R | |
| 39 | 39 | \| | Q | Q | Q | Q | Q | |
| 40 | 40 | \| | R | R | A | A | A | |
| 41 | 41 | \| | P | P | P | P | P | |
| 42 | 42 | \| | E | E | G | G | G | |
| 43 | 43 | \| | Q | Q | Q | Q | Q | |
| 44 | 44 | \| | G | G | G | R | R | V_{L}-V_{H} packing, |
| | | | | | | | | consider G in other |
| | | | | | | | | version |
| 45 | 45 | \| | L | L | L | L | L | |
| 46 | 46 | \| | E | E | E | E | E | |
| 47 | 47 | \| | W | W | W | W | W | |
| 48 | 48 | \| | I | I | M | M | M | |
| 49 | 49 | FR2 | G | G | G | G | G | |
| 50 | 50 | CDR2 | R | R | W | W | R | |
| 51 | 51 | \| | I | I | I | I | I | |
| 52 | 52 | \| | D | D | N | N | D | |
| 52A | 53 | \| | P | P | P | A | P* | |
| 52B | | \| | — | — | — | — | — | |
| 52C | | \| | — | — | — | — | — | |
| 53 | 54 | \| | A | A | G | G | A* | |
| 54 | 55 | \| | N | N | N | N | N* | |
| 55 | 56 | \| | G | G | G | G | G* | |
| 56 | 57 | \| | Y | N | D | N | Y | |
| 57 | 58 | \| | T | T | T | T | T | |
| 58 | 59 | \| | K | K | N | K | K | |
| 59 | 60 | \| | Y | Y | Y | Y | Y | |
| 60 | 61 | \| | D | D | A | S | D | |
| 61 | 62 | \| | P | P | Q | Q | P | |
| 62 | 63 | \| | K | K | K | K | K | |
| 63 | 64 | \| | F | F | F | F | F | |
| 64 | 65 | \| | Q | Q | Q | Q | Q | |
| 65 | 66 | CDR2 | G | G | G | G | G | |
| 66 | 67 | FR3 | K | K | R | R | R | |
| 67 | 68 | \| | A | A | V | V | V | |
| 68 | 69 | \| | T | T | T | T | T | |
| 69 | 70 | \| | I | I | I | I | I | |
| 70 | 71 | \| | T | T | T | T | T | |
| 71 | 72 | \| | A | A | A | R | A* | H2 canonical structure, |
| | | | | | | | | supporting H2 |
| 72 | 73 | \| | D | D | D | D | D | |
| 73 | 74 | \| | T | T | T | T | T | |
| 74 | 75 | \| | S | S | S | S | S | |
| 75 | 76 | \| | S | S | T | A | A | |
| 76 | 77 | \| | N | N | S | S | S | |
| 77 | 78 | \| | T | T | T | T | T | |
| 78 | 79 | \| | A | A | A | A | A | |
| 79 | 80 | \| | Y | Y | Y | Y | Y | |
| 80 | 81 | \| | L | L | M | M | M | |
| 81 | 82 | \| | Q | Q | E | E | E | |
| 82 | 83 | \| | L | L | L | L | L | |
| 82A | 84 | \| | S | S | S | S | S | |
| 82B | 85 | \| | S | S | S | S | S | |
| 82C | 86 | \| | L | L | L | L | L | |
| 83 | 87 | \| | T | T | R | R | R | |
| 84 | 88 | \| | S | S | S | S | S | |
| 85 | 89 | \| | E | E | E | E | E | |
| 86 | 90 | \| | D | D | D | D | D | |
| 87 | 91 | \| | T | T | T | T | T | |
| 88 | 92 | \| | A | A | A | A | A | |
| 89 | 93 | \| | V | V | V | V | V | |
| 90 | 94 | \| | Y | Y | Y | Y | Y | |
| 91 | 95 | \| | F | Y | Y | Y | Y | |
| 92 | 96 | \| | C | C | C | C | C | |
| 93 | 97 | \| | A | A | A | A | A | |
| 94 | 98 | FR3 | R | R | R | R | | |
| 95 | 99 | CDR3 | E | G | A | G | E | |
| 96 | 100 | \| | G | Y | P | G | G | |
| 97 | 101 | \| | Y | Y | G | Y | Y | |
| 98 | 102 | \| | Y | Y | Y | Y | Y | |
| 99 | 103 | \| | G | Y | G | G | G | |
| 100 | 104 | \| | N | D | S | S | N | |
| 100A | 105 | \| | Y | S | G | G | Y | |
| 100B | 106 | \| | G | X | G | S | G | |
| 100C | 107 | \| | V | V | O | — | V | |
| 100D | 108 | \| | Y | G | C | — | Y | |
| 100E | 109 | \| | A | Y | Y | — | A | |
| 100F | 110 | \| | M | Y | R | M | | |
| 100G | | \| | — | A | 0 | — | — | |
| 100H | | \| | — | M | D | — | — | |
| 100I | | \| | — | — | Y | — | — | |
| 100J | | \| | — | — | — | — | | |
| 100K | | \| | — | — | F | — | — | |
| 101 | 111 | \| | D | D | D | N | D | |
| 102 | 112 | CDR3 | Y | Y | Y | Y | Y | |
| 103 | 113 | FR4 | W | W | W | W | W | |
| 104 | 114 | \| | G | G | G | G | G | |
| 105 | 115 | \| | Q | Q | Q | Q | Q | |
| 106 | 116 | \| | G | G | G | G | G | |
| 107 | 117 | \| | T | T | T | T | T | |
| 108 | 118 | \| | S | X | L | L | L | |
| 109 | 119 | \| | V | V | V | V | V | |
| 110 | 120 | \| | T | T | T | T | T | |
| 111 | 121 | \| | V | V | V | V | V | |
| 112 | 122 | \| | S | S | S | S | S | |
| 113 | 123 | FR4 | S | S | S | S | S | |

Legend:
(Kabat) numbering according to Kabat et al., supra;
(#) sequential numbering as used in the molecular modeling; (mouse 21.6) amino acid sequence of the V_{H} region from mouse 21.6 antibody;
(mouse 2c) consensus sequence of mouse V_{H} regions from subgroup 2c (Kabat et al., supra);
(human 1) consensus sequence of human V_{H} regions from subgroup 1 (Kabat et al., supra);
(human 21/28′CL) amino acid sequence of a human V_{H} region (Dersimonian et al., J. Immunol., 139: 2496-2501 (1987));
(RH V_{H} 21.6) amino acid sequence of version H1 of reshaped human 21.6 V_{H} region;
*residues that are part of the canonical structures for the CD loops (Chothia et al., supra);
(underlined) residues in the human FRs where the amino acid residue was changed.

### Example 2

Natalizumab

Natalizumab is a recombinant humanized antibody (rhAb) directed against the α4 integrin molecule and inhibits cell binding mediated by α4β1 (VLA-4) and α4β7 integrins. Natalizumab binds to the α4 subcomponent, which is expressed on leukocytes, predominantly lymphocytes. The binding of the murine monoclonal antibody to α4 integrin blocks the interaction of α4β1 on these leukocytes with its counter receptor on endothelial cells, VCAM-1. The blockade of these cell adhesion molecule interactions is believed to prevent the trafficking of these leukocytes across the vascular endothelium and, subsequently, into the parenchymal tissue.

α₄ integrins bind additional ligands in tissues, including osteopontin and epitopes of fibronectin. A further mechanism of natalizumab includes the suppression of ongoing inflammatory reactions in diseased tissues by inhibition of α4-positive leukocytes with these ligands. Thus, natalizumab acts to suppress existing inflammatory activity present at the disease site, along with inhibition of further recruitment of immune cells into inflamed tissue via interaction with VCAM-1 and MadCAM-1.

Work in inflammatory bowel disease (IBD) has demonstrated the expression of vascular cell adhesion molecule-1 (VCAM-1) and mucosal addressin cell adhesion molecule (MadCAM-1) at active sites of inflammation in both inflamed and non-inflamed bowel of IBD subjects, which suggests that recruitment of leukocytes to the mucosa contributes to the inflammatory response characteristic of IBD. Therefore, an agent which disrupts VCAM-1/α4β1 and MadCAM-1/α4β7 interactions could result in reduction of lymphocyte migration and attenuate the release of cytokines and other substances which cause tissue injury. Studies of anti-α4 integrin antibodies in the cotton-top tamarin (CTT), a primate species that experiences a form of chronic IBD which has a similar pattern of expression of key adhesion molecules in inflamed bowel tissue, have shown highly significant improvement in acute colitis in comparison to placebo.

Single- and multiple-dose toxicity studies have been performed in mice, guinea pigs, and monkeys. All toxicology studies were carried out using natalizumab and included an acute study in guinea pigs, subacute studies in mouse and cynomolgus monkeys, and mutagenicity and tissue cross-reactivity studies. These studies did not demonstrate clinical or postmortem evidence of significant toxicity.

In mice, there is evidence that α4 integrin and VCAM-1 play a role in placental and cardiac development, and they may also play a wider role in fetal development. There is, therefore, a risk of an abortifacient effect or teratogenicity if α4 integrin is blocked by natalizumab. A preliminary reproductive toxicity study exposed groups of five pregnant cynomolgus monkeys to repeated intravenous doses of 0.06, 0.3, or 30 mg/kg natalizumab. One of five pregnant females in the 30 mg/kg group aborted at Day 31 of gestation after receiving five doses of natalizumab. Because the overall rate of abortion fell within the rate of spontaneous abortion in this species, the event was not believed to be related to natalizumab. An ongoing follow-up reproductive toxicity study has exposed groups of 10 to 15 pregnant cynomolgus monkeys to repeated intravenous doses of 3, 10, or 30 mg/kg. Embryo deaths have occurred at similar rates in all treatment groups: two in the control, one in the 3 mg/kg, two in the 10 mg/kg and two in the 30 mg/kg groups respectively. As a precaution, women of childbearing potential must utilize effective contraception throughout the duration of the study and for at least 3 months after the last infusion of study drug, and must have a negative pregnancy test at the time of each natalizumab dosing.

In the six-month multidose toxicity study in primates, minimal to mild lymphoplasmacytic inflammation of the mucosa of the cecum, colon, and/or rectum was noted in about half of the natalizumab-treated animals of all dose groups and was not found in the vehicle-group. The inflammation was characterized by increased numbers of lymphocytes and plasma cells within the lamina propia with occasional crypt abscesses. There was a slightly increased incidence and magnitude of the change in the colon and rectum of animals from the natalizumab 30.0 and 60.0 mg/kg/week groups, indicating a dose-response relationship. However, although there was a possible dose-response relationship, the incidence of inflammation was not related to the natalizumab serum levels. While these changes may reflect some underlying infection of the intestinal tract in the affected animals, the slightly increased incidence and magnitude of the inflammation in the animals of the two highest natalizumab dose groups, combined with the lack of its presence in the control group, indicates natalizumab may possibly have a role in this process.

Earlier studies in Crohn's disease and ulcerative colitis are summarized below. One study was a randomized, double-blind, placebo-controlled, safety, tolerability, and efficacy study of a single infusion of intravenous 3 mg/kg natalizumab in male and female subjects diagnosed with chronic active Crohn's disease. Thirty subjects were enrolled; 18 were treated with natalizumab (3 mg/kg) and 12 with placebo. Two weeks following treatment, 7 natalizumab-treated subjects (39%) and 1 placebo-treated subject (8%) were in clinical remission (Crohn's disease Activity Index (CDAI)<150) (p=0.1). In addition, at Week 2 post-treatment, fewer natalizumab-treated subjects (11%) required rescue therapy compared to placebo-treated subjects (33%). Mean CDAI scores were significantly decreased at both 2 and 4 weeks post-treatment in the natalizumab group only, compared to mean baseline CDAI scores. These effects were not sustained beyond 4 weeks post-treatment and correlate with low natalizumab serum concentrations observed at the Week 4 timepoint.

Natalizumab treatment with a single, intravenous dose of 3 mg/kg was safe and well tolerated by subjects with CD. No subjects were withdrawn from the study because of the occurrence of an adverse event. Six subjects reported one serious adverse event; all subjects were in the natalizumab-treated group. None of these events were fatal. Five of the six events were admissions for relapses or worsening of the subject's Crohn's disease, the other serious adverse event was admission for anemia. There was no significant difference between natalizumab and placebo groups in the incidence of the most frequently reported adverse events (headache, Crohn's disease and abdominal pain).

A second earlier study was an open-label safety, tolerability, and efficacy study of a single infusion of 3 mg/kg intravenous natalizumab in male and female subjects with active ulcerative colitis. Ten subjects were recruited and treated with natalizumab (3 mg/kg).9 At 2 and 4 weeks post-treatment, 5 subjects (50%) had a good clinical response, defined as a Powell-Tuck Activity Index (PTAI) score of ≦5 and mean PTAI scores decreased from 9.7 at Week 0 to 6.9, 5.7, and 4.9 at 1, 2, and 4 weeks post-treatment, respectively. The mean PTAI scores remained suppressed for the 12-week study period. Seventy percent of subjects received no rescue medication between Weeks 0 and 4.

The most frequently reported adverse events in this study were aggravation of ulcerative colitis, headache, vomiting, lethargy, and sore throat. Of the 30 adverse events reported in this study, only 3 were considered by the Investigator to be related to study drug. These were one incidence each of headache, aggravation of ulcerative colitis, and lethargy. There were two events characterized by the Investigator as severe, both events were reports of aggravated ulcerative colitis not considered to be related to treatment. Three subjects reported a serious adverse event. None of these events were fatal. These events were an incidence of Campylobacter enteritis; a relapse of ulcerative colitis, which resulted in the subject withdrawing from the study; and an episode of rigors, fever, headache, and vomiting.

Another earlier study was a double-blind, placebo-controlled, parallel group, multicenter, efficacy, safety, and tolerability study of either one or two intravenous infusions of placebo, 3 or 6 mg/kg natalizumab in subjects with moderately to severely active Crohn's disease. A total of 248 subjects were randomized of whom 244 received at least one dose of study drug. Sixty-eight subjects were randomized to a single infusion of 3 mg/kg, 66 to two infusions of 3 mg/kg at a 4-week interval, 51 to two infusions of 6 mg/kg at a 4-week interval and 63 to receive placebo. Natalizumab was superior to placebo in inducing remission (CDAI<150) in at least one of the three active treatment groups at Weeks 4, 6, 8, and 12. The highest remission rate of 46% was observed at Week 6 in the group that received two infusions of 3 mg/kg, remission rates of 41-43% were observed at Weeks 8 and 12 in this and the group that received two infusions of 6 mg/kg. Natalizumab was superior to placebo in inducing a response (≧70 point or ≧100 point drop in CDAI) in at least one of the three active treatment groups at Weeks 2, 4, 6, 8, and 12. The highest response rates of 73% (≧70 point drop) and 56% (≧100 drop) was observed at Week 6 in the group that received two infusions of 3 mg/kg. Statistically significant improvements in quality of life, assessed through the Inflammatory Bowel Disease Questionnaire, and decreases in C-reactive protein were also achieved.

Treatment with natalizumab appeared safe and well tolerated by subjects with active CD. Similar numbers of subjects from each treatment group withdrew due to adverse events: 2, 1, 2, and 3 subjects in the placebo, single 3.0 mg/kg, two 3 mg/kg and two 6 mg/kg infusion dose groups, respectively. A total of 32 subjects reported a serious adverse event during the main phase of the study (9, 8, 8, and 7 subjects in the placebo, single 3.0 mg/kg, two 3 mg/kg, and two 6 mg/kg infusion dose groups, respectively). None of these events were fatal and none were assessed as related to study drug. The majority of these events were admissions for treatment of complications or symptoms of CD. The non-disease-related events which were reported with greater frequency in at least two of the natalizumab treatment groups included chest pain, fever, flu syndrome, dizziness, and conjunctivitis.

Use of Natalizumab in Treatment of Crohn's Disease

The majority of subjects with CD will initially respond to the available medications including 5-ASA formulations (sulfasalazine, mesalazine, olsalazine), oral steroids (e.g., prednisolone, methylyprednisolone, budesonide). More recently, agents directed against tumor necrosis factor (the anti-TNF alpha antibody, infliximab) for the treatment of severe refractory CD and refractory fistulizing disease have been developed. However, some patients continue to have debilitating disease and there is a need for an improved treatment for subjects whose disease is not well controlled by current therapy.

There is evidence of up-regulation of MadCAM-1 and VCAM-1 in subjects with IBD, with evidence that the MadCAM-1/α4β7 interaction mediates the homing of lymphocytes to the gut. The potential role of anti-α4 integrin antibodies in IBD was initially supported by findings from studies in the cotton-top tamarin and more recently by the results of natalizumab in clinical trials.

Two further studies, described in detail below, were planned to confirm the earlier results and is designed to induce response and/or remission in a population of moderately to severely active CD subjects (CDAI≧220, ≦450). Subjects from the first study who responded and then had mildly active disease (CDAI score<220 and ≧70 drop) were enrolled in a subsequent study, which was designed to determine whether repeated administration of natalizumab can maintain response and/or remission. Given the chronic nature of CD it is clearly important that new agents are evaluated for their ability to reduce or eliminate disease activity over a longer period of time. In addition, the approach of maintaining an improvement once achieved also reflects aims of current clinical practice.

The primary tool for the assessment of efficacy is the Crohn's disease Activity Index (CDAI). The CDAI was developed for the US National Co-operative Crohn's Study (NCCDS) in 1979 and is the best known of the CD clinical scores. It is widely used in clinical trials of new therapies and has gained general acceptance as an endpoint for clinical activity. 10 A CDAI score of <150 is generally accepted as remission, scores of ≧150 to <220 are considered mildly active disease whilst scores of ≧220 to <450 are considered moderately to severely active disease.

Accordingly, a loss of response is defined as a CDAI score of ≧220 and a loss of remission as a CDAI score of ≧150. These definitions in combination with the use of rescue intervention, were used in the maintenance of response and remission analyses in this study.

Additional endpoints for this study included assessment through the Inflammatory Bowel Disease Questionnaire, a quality of life tool that has been developed for the IBD population, and the SF-3612 which affords a more generic assessment of quality of life and which is favored by some regulatory authorities. Changes in inflammatory markers such as C-reactive protein were also assessed, as will the ability to withdraw concomitant oral steroids in the sub group of subjects receiving them.

The initial dose of natalizumab selected for clinical evaluation was based on non-clinical studies in the guinea pig Experimental Allergic Encephalitis (EAE) model. These studies demonstrated that a dose of 3 mg/kg of natalizumab produced both a significant delay in onset and a reversal of the signs and symptoms of EAE; lower doses of natalizumab were not effective. A single dose of 3 mg/kg appeared to provide serum concentrations of natalizumab associated with α4 integrin receptor blockade for up to approximately 3 weeks, and 6 mg/kg for about 6 weeks.

Natalizumab has been evaluated in all clinical trials to date by administration of dose adjusted for bodyweight. Single dose pharmacokinetic data from completed clinical trials in healthy volunteers and in subjects with MS and IBD showed a 3 mg/kg infusion of natalizumab can maintain natalizumab serum concentrations of 2.5-3.0 μg/mL levels that are associated with a sufficient degree of receptor saturation and the inhibition of cell adhesion for 3-4 weeks. A single infusion of a higher dose of 6 mg/kg natalizumab produced α4 integrin saturation levels which were slightly higher and more prolonged (approximately 6 weeks).

The pharmacodynamic effects and therapeutic response observed in these Phase II studies were found to be related to natalizumab dose and serum natalizumab concentrations. Based on these findings, along with the knowledge that natalizumab clearance is largely independent of bodyweight, the range of exposures produced by a fixed dose of 300 mg was investigated to determine if fixed dose administration could replace dosing adjusted by bodyweight.

Through pharmacokinetic modeling and assuming that AUC is proportional to total dose, it was demonstrated that a 300 mg fixed dose will produce natalizumab exposures that overlap the exposures observed for the 3 mg/kg and 6 mg/kg doses used in the Phase II trials. Thus, since the 3 mg/kg dose was efficacious in both CD and MS indications, the 6 mg/kg dose resulted in no evidence of dose-limiting toxicities and there was no added benefit of the 6 mg/kg dose over the 3 mg/kg dose, a 300 mg fixed dose is an appropriate choice for Phase III studies.

A double-blind, placebo-controlled study of the efficacy, safety, and tolerability of intravenous Antregren™ (natalizumab, 300 mg monthly) in maintaining clinical response and remission in patients with Crohn's Disease (CD) was performed. The objectives were to compare the ability of natalizumab versus placebo to maintain a clinical response in subjects with CD, to compare the ability of natalizumab versus placebo to maintain a clinical remission in subjects with CD, to compare the effects of natalizumab versus placebo on quality of life as measured by the Inflammatory Bowel Disease Questionnaire (IBDQ), and to compare the ability of natalizumab versus placebo to allow subjects to achieve withdrawal of oral steroids.

Study Design

A Phase III, international, multicenter, randomized, double-blind, placebo-controlled, parallel-group study of subjects with previously active Crohn's disease (CD) (defined as moderately to severely active, CDAI≧920, <450) who have responded to treatment at Week 10 and maintained that response out to Week 12 in a first study (defined as a ≧70 point decrease in baseline CDAI) and whose disease is mildly active (defined as a CDAI score of <220) was undertaken.

Within this group there was a sub-population who had achieved remission (defined as a CDAI score of <150) at Week 10 of the first study. Subjects who failed to maintain response from Week 10 to Week 12 were not be eligible for the second study and continued in the safety follow-up phase of the first study. Subjects receiving concomitant medications for their CD were permitted to enroll providing that doses remained stable throughout their participation in the first study. All concomitant medications for CD remained stable for the duration of the 12-month treatment phase (up to Month 15) with the exception of oral steroids which were reduced according to a fixed algorithm). At the time this application was filed, only 9 months of data was available.

Natalizumab was administered 300 mg monthly for 12 infusions. The placebo was administered monthly for 12 infusions. Subjects were stratified according to their disease status (remission versus no remission, ie., a CDAI<150 or ≧2150), concomitant use of oral steroids and concomitant use of immunosuppressants.

Once randomized, subjects received their first infusion. Thereafter, they returned to the clinic on a monthly basis (where 1 month is defined as a 4-week period) for assessment and infusion. Introduction of any new medication for CD or a dose change to an existing concomitant medication for CD (with the exception of oral steroids withdrawn according to the fixed algorithm) was be permitted unless deemed necessary for purposes of rescue intervention. Once rescued, such a subject was considered a treatment failure.

Subjects will receive up to 12 infusions in this study and will return for the final treatment phase assessment 1 month after the last infusion (i.e., at Month 15).

Sample Size

Approximately 380 subjects were expected to respond to treatment and have mildly active disease at Week 10 in the first study (defined as ≧70 point decrease in CDAI score and a CDAI score of <220 and no use of rescue intervention), maintained to Week 12/Month 3. 285 were expected to enroll into the second study, assuming a 25% drop-out rate of eligible subjects between the two studies. Of these, 200 subjects were expected to have achieved remission (defined as CDAI score of <150).

A sample size of 285 subjects randomized and dosed (142 per treatment group; ratio 1:1) were given a power of 90% at 5% significance to detect a difference between the natalizumab-treated group and the placebo group in maintenance of response rates (defined as a CDAI score of <220 and no use of rescue intervention), assuming a 65% response rate for natalizumab and a 44% response rate for placebo and allowing for a 10% drop-out rate.

Accordingly, the sub group of 200 subjects in remission, randomized and dosed were given a power of 90% at 5% significance to detect a difference between the natalizumab-treated group and the placebo group in maintenance of remission (defined as a CDAI score of <150 and no use of rescue intervention), assuming a 55% response rate for natalizumab and a 30% response rate for placebo and allowing for a 10% drop-out rate.

Eligible subjects at Week 10 in the first study were consented and enrolled into the second study, in order to allow subjects taking concomitant oral steroids to begin a steroid taper. Subjects who continued to meet the eligibility criteria at Week 12/Month 3, ie., in time for their next, monthly infusion, were re-randomized and entered the treatment phase which will last up to 12 months in duration (i.e., up to Month 15). Subjects were male or female, eighteen years of age or older.

Drug Dosage and Formulation

Intravenous natalizumab was administered at a dose of 300 mg. Natalizumab was provided in 5 mL vials at a concentration of 20 mg/mL. All infusions were made up in 100 mL bags of 0.9% saline. Natalizumab vials contained 20 mg/mL natalizumab in 10 mM phosphate buffer, 140 mM NaCl and a 0.02% polysorbate 80, adjusted to pH 6.0 with phosphoric acid.

For the control group, placebo was provided in matching 5-mL vials and comprised 10 mM phosphate buffer, 140 mM NaCl and 0.02% polysorbate 80, adjusted to a pH of 6.0 with phosphoric acid.

Route of Administration

Natalizumab or placebo was administered by intravenous infusion over approximately 60 minutes, at a flow rate of 2 mL/min. All subjects will be observed for 2 hours post the start of each infusion.

Procedures

Procedures included physical examination, vital signs, bodyweight, CDAI, IBDQ, SF-36, Subject Global Assessment, blood samples for assessment of hematology, biochemistry, C-reactive protein (CRP), anti-nuclear antibodies (ANA), serum natalizumab levels, anti-natalizumab antibodies and pregnancy testing, urine samples for urinalysis and pregnancy testing, assessment of adverse event, concomitant medications and rescue intervention.

Primary, Secondary and Tertiary Endpoints

Primary Endpoint:

The primary endpoint was time to loss of response (defined as a CDAI score 220 or use of rescue intervention) for subjects in response at Week 12.

Secondary Endpoints:

1. Contingent Primary Endpoint: Time to loss of remission (defined as a CDAI score 150 or use of rescue intervention) for those subjects in remission at Week 12 (defined as a CDAI score<150).
2. Proportion (%) of those subjects in remission at Week 12 (defined as a CDAI score<150) who remained in remission (defined as a CDAI score<150 AND no use of rescue intervention) after 12 months (i.e., at Month 15).
3. Mean change in IBDQ from baseline in CD301, at Month 9.
4. Number (%) not taking oral steroids, at Month 9. Number (%) of subjects in remission (defined as a CDAI score<150 AND no use of rescue intervention) and not taking oral steroids, at Month 9.
   Tertiary Endpoints:
1. Number (%) of subjects with mildly active disease (defined as a CDAI score of <220 AND no use of rescue intervention), at Months 3, 4, 5, 6, 7, 8, 9, 10, 11, 12, 13, 14, and 15.
2. Number (%) of subjects in remission (defined as a CDAI score<150 AND no use of rescue intervention), at Months 3, 4, 5, 6, 7, 8, 9, 10, 11, 12, 13, 14, and 15.
3. Number (%) of subjects with CDAI score<200 AND no use of rescue intervention, at Months 3, 4, 5, 6, 7, 8, 9, 10, 11, 12, 13, 14, and 15.
4. Mean CDAI scores at Months 3, 4, 5, 6, 7, 8, 9, 10, 11, 12, 13, 14, and 15.
5. Mean change in IBDQ from baseline in CD301, at Months 3, 6, 12 and 15.
6. Mean change in SF36 from baseline in CD301, at Months 3, 6, 9, 12 and 15.
7. Mean change in Subject Global Assessment from baseline in CD301, at Months 3, 6, 9, 12, and 15.
8. Number (%) not taking oral steroids, at Months 3, 4, 5, 6, 7, 8, 10, 11, 12, 13, 14, and 15.
9. Number (%) of subjects in remission (defined as a CDAI score<150 AND no use of rescue intervention) and not taking oral steroids, at Months 3, 4, 5, 6, 7, 8, 10, 11, 12, 13, 14, and 15.
10. Time to first use of rescue intervention (including surgical intervention).
11. Number (%) of subjects requiring rescue intervention (including surgical intervention), at Months 3, 4, 5, 6, 7, 8, 9, 10, 11, 12, 13, 14, and 15.
12. Mean change in C-reactive protein from baseline in CD301 (in those subjects who had an elevated CRP at baseline in CD301), at Months 3, 6, 9, 12, and 15.
13. Mean change in serum albumin from screening in CD301, at Months 3, 6, 9, 12, and 15.
   Statistical Considerations

The efficacy analyses was based on the intention-to-treat population. The analysis of the primary endpoint was repeated for a per protocol population. Categorical data was presented as counts and percentages. Continuous data was presented as summary statistics. All comparisons made were two-tailed at the 5% level of significance.

The primary analyses adjusted for the factors used for the stratification as well as geographical location and other pre-specified covariates. The Contingent Primary Endpoint, time to loss of remission, were only analyzed if the primary efficacy endpoint was statistically significant at the 5% level.

An administrative analysis will be carried out when 400 patient years of data are available from the first and second studies combined and when every subject will have a minimum of 3 months of data (i.e., have completed the first study to Week 12 as a minimum or have completed Week 12/Month 3 in the second study).

Study Drug

Study drug was provided (either natalizumab or placebo) in clear, stoppered, individual 5 mL vials of either 20 mg/mL natalizumab or placebo. Vials were packaged in boxes of 3 vials to protect them from light. Each box was labeled with a unique 6 digit Box Number and will contain sufficient study drug for one infusion. Natalizumab vials contained 20 mg/mL natalizumab in 10 mM phosphate buffer, 140 mM NaCl and 0.02% polysorbate 80, adjusted to pH 6.0 with phosphoric acid. Placebo was provided in matching 5 μL vials and comprises 10 mM phosphate buffer, 140 mM NaCl and 0.02% polysorbate 80, adjusted to pH 6.0 with phosphoric acid.

Study Drug Dosage

Subjects randomized to natalizumab received 300 mg monthly (i.e., every 4 weeks) via an intravenous infusion for up to 12 infusions.

Study Drug Preparation

Fifteen milliliters of saline, a volume equivalent to that of study drug to be added, were withdrawn from the 100 mL bag of 0.9% saline and discarded. A total of 15 mL of study drug was then drawn from the three vials into a 50 mL syringe via an 18 or 19 gauge needle, and gently added into the saline bag via a 20-23 gauge needle through the medication port diaphragm. The dilution was performed using aseptic technique.

Study Drug Administration

Administration of study drug occurred every 4 weeks by intravenous infusion. Each infusion took approximately 60 minutes. The infusion was performed using a flow rate of approximately 2 mL/min. Once the 100 mL bag of study drug is empty, it was replaced with the 50 mL bag of saline, in order to flush the infusion line at the same rate.

Oral Steroid Tapering

All subjects receiving oral steroids were required to undergo a taper immediately upon entry into the second study using the following algorithm. Subjects on doses equivalent to >10 mg of prednisolone will begin their taper at a rate of 5 mg every 7 days until they reach a dose of 10 mg. Subjects on doses equivalent to 10 mg of prednisolone were tapered at a rate of 2.5 mg every 7 days until they are completely withdrawn. Subjects taking budesonide were tapered at a rate of 3 mg every 3 weeks.

Physical Examination

Complete physical examinations were and will be performed at Month 6, Month 9, Month 12, and Month 15 and as part of the Early Discontinuation Visit for subjects who withdrew before Month 15. Bodyweight was recorded at every visit as part of the assessment of the CDAI score. Vital signs were recorded at every visit. At visits when infusions are administered, vital signs were recorded immediately before (0 minutes) and at the end of the infusion.

Quality of Life Assessments

Quality of life assessments, consisting of Subject Global Assessment, Inflammatory Bowel Disease Questionnaire (IBDQ), and a health survey were completed by the subject during the Month 3, Month 6, Month 9, Month 12 and Month 15 visits and as part of the Early Discontinuation Visit. Subjects must complete assessments at the beginning of the visit (i.e., before any other assessments or the infusion), completing the Subject Global Assessment first. The Subject Global Assessment is a visual analog scale on which the subject assessed their global impression of how they feel compared to how they felt immediately prior to receiving their first administration of study medication.

Natalizumab Concentration

Blood samples for natalizumab concentration measurement were collected during the Month 3, Month 6, Month 9, Month 12, Month 15. At visits when infusions were administered, the sample were taken immediately before (0 minutes) the infusion. In addition, at Month 6 and Month 12 only, a second sample was collected at least 1 hour from the end of the infusion.

Statistical Methods

The ability of natalizumab to maintain mildly active disease (defined as a CDAI score of <220 and no use of rescue intervention) and remission (defined as a CDAI score of <150 and no use of rescue intervention) in subjects with CD was assessed. The primary comparison of interest was the time to loss of response between treatment groups (where loss of response is defined as a CDAI score 220 or use of rescue intervention). A contingent sequential analysis was done on the effect of treatment on the time to the loss of remission between treatment groups (defined as CDAI score 150 or use of rescue intervention) in the sub-group of subjects in remission (CDAI<150) at Week 12.

Approximately 380 subjects were expected to respond to treatment and have mildly active disease at Week 10 in the first study (defined as ≧70 point decrease in CDAI score and a CDAI score of <220 and no use of rescue intervention) which is maintained to Week 12/Month 3. Of which, 285 were expected to enroll into the second study, assuming a 25% drop-out rate of eligible subjects between the two studies. Of these, 200 subjects were expected to have achieved remission (defined as CDAI score of <150).

A sample size of 285 subjects randomized and dosed (142 per treatment group; ratio 1:1) were given a power of 90% at 5% significance to detect a difference between the natalizumab-treated group and the placebo group in maintenance of response rates (defined as a CDAI score of <220 and no use of rescue intervention), assuming a 65% response rate for natalizumab and a 44% response rate for placebo and allowing for a 10% drop-out rate.

Accordingly, the sub group of 200 subjects in remission, randomized and dosed were given a power of 90% at 5% significance to detect a difference between the natalizumab-treated group and the placebo group in maintenance of remission (defined as a CDAI score of <150 and no use of rescue intervention), assuming a 55% response rate for natalizumab and a 30% response rate for placebo and allowing for a 10% drop-out rate.

Efficacy Analysis

All efficacy analyses and summaries were based on the intention-to-treat population. A confirmatory analysis of the primary efficacy parameter was carried out using the per protocol population. A sensitivity analysis was carried out on the primary and secondary endpoints using a subset of the intention-to-treat population comprising of those responders from the first study who were randomized to receive natalizumab in the first study.

Results

In the second study, subjects not taking steroids at month 9 (of those who took steroids at the first study baseline) showed the following results:

TABLE 18

| | Placebo | | |
|---|---|---|---|
| | N = 76o | Natalizumab | p-value |
| Not taking steroids | 19 (25%) | 37 (55%) | <0.001 |
| Remission and not | 17 (22%) | 31 (46%) | 0.009 |
| taking steroids | | | |

The most common side effects in both groups were headache, nausea and abdominal pain. Of serious adverse events (SAEs), 8% versus 7% placebo versus natalizumab.

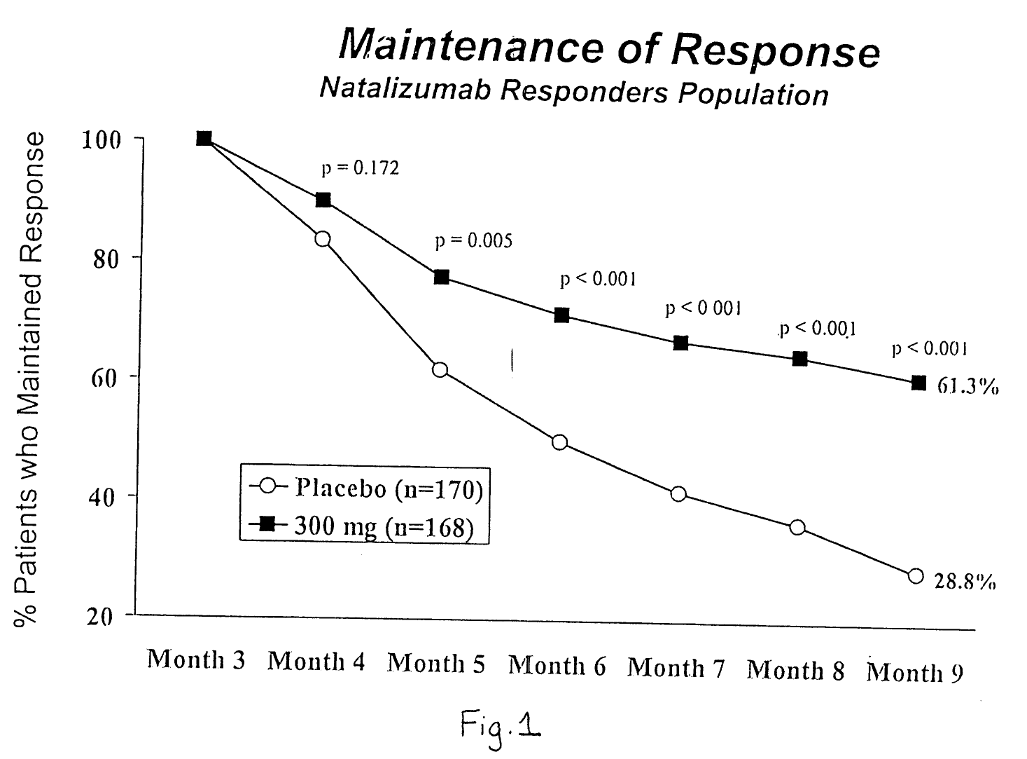

FIG. 1 shows a graph of the response to natalizaumab when given to patients in a Crohn's disease trial. Of the natalizumab responder population at three months into the trial, 61.3% of the patients maintained a response after 9 months, while only 28.8% of the placebo group maintained a response.

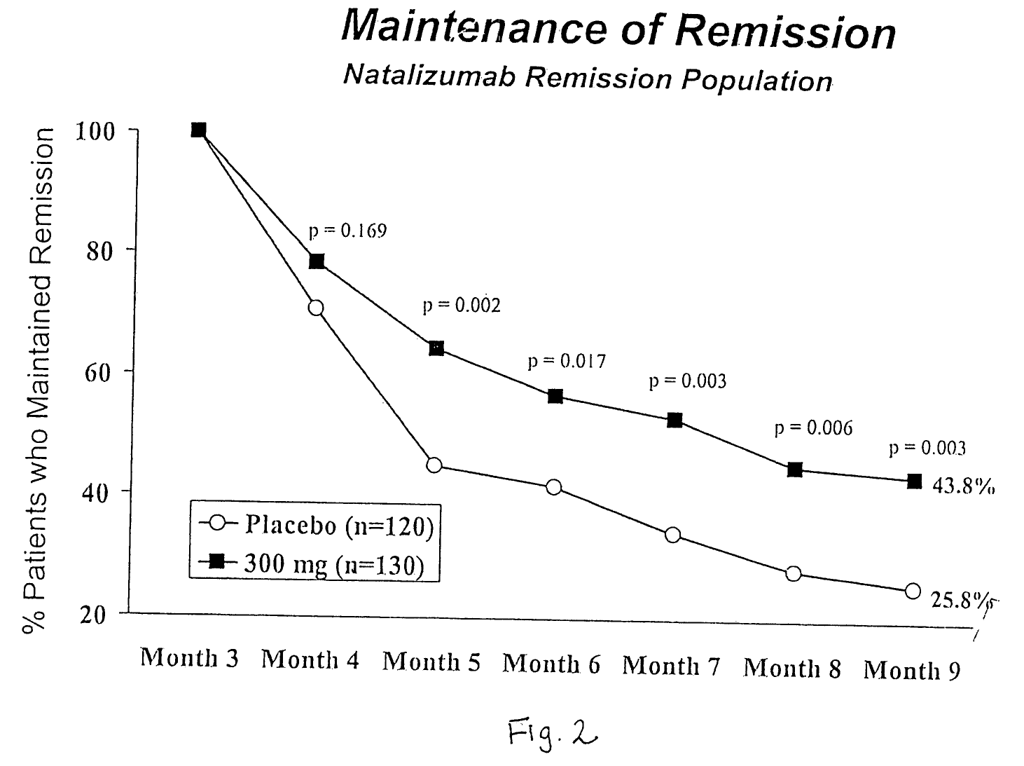

FIG. 2 shows a graph of the level of remission in response to natalizaumab when given to patients in a Crohn's disease trial. Of the natalizumab remission population at three months into the trial, 43.8% of the patients maintained a response after 9 months, while only 25.8% of the placebo group maintained a response.

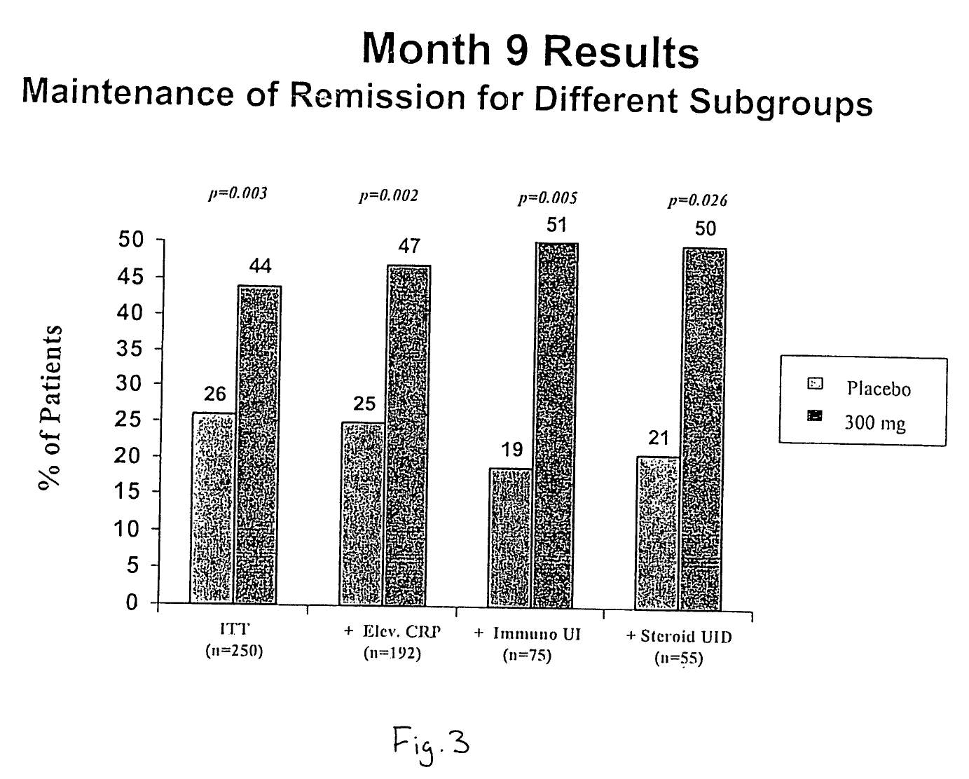

FIG. 3 shows a graph of the level of remission in response to natalizumab when given to patients in a Crohn's disease trial (see Example 2) in various populations: the intention-to-treat population (ITT), elevated C-reactive protein population (CRP), the population unresponsive or intolerant to immunosuppressives (immuno UI). and the population unresponsive, intolerant to, or dependent upon steroids (steroid UID). These categorizations were based upon patient history of previous use of these medications.

Efficacy Summary

In populations of interest, clnically meaningful differences in remission and response rates were observed with natalizumab compared to placebo in the first study. The second study, (double blind withdrawal study of responders in the first study) confirms the induction effect seen in the first study. Encouraging maintenance data observed after 6 months in the second study. In the second study, natalizumab enabled subjects to be successfully tapered off steroids.

TABLE 19
CROHN'S DISEASE ACTIVITY INDEX (CDAI)

| | Weighting |
|---|---|
| Variable | factor |
| Total number of diarrheal stools for each of previous 7 days | ×2 |
| Abdominal pain for each of previous 7 days | ×5 |
| None = 0 | |
| Mild = 1 | |
| Moderate = 2 | |
| Severe = 3 | |
| General well-being for each of previous 7 days | ×7 |
| Well = 0 | |
| Below par = 1 | |
| Poor = 2 | |
| Very poor = 3 | |
| Terrible = 4 | |

All other indices will be assessed by the Doctor at
outpatient visit as follows:

| | |
|---|---|
| Clinical signs during the previous 7 days | ×20 |
| Arthritis or arthralgia = 1 | |
| Skin or mouth lesions = 1 | |
| Iritis or uveitis = 1 | |
| Anorectal lesion = 1 | |
| Other fistulae = 1 | |
| Fever over 38° C. during the week = 1 | |
| Lomotil or other anti-diarrheal | ×30 |
| No = 0, yes = 1 | |
| Abdominal mass | ×10 |
| None = 0 | |
| Questionable = 2 | |
| Definite = 5 | |
| Anemia defined by hematocrit less than: | ×6 |
| For males-47% | |
| For females-42% | |
| Standard weight-Actual weight × 100 | ×1 |
| Standard weight* | |
| Crohn's disease Activity Index (CDAI) Total | = |

*Obtain from the Standard Height and Weight Tables which will be provided.

### Example 3

This experiment was a double-blind, placebo-controlled study of the efficacy, safety, and tolerability of natalizumab in maintaining clinical response and remission in Crohn's Disease.

Natalizumab, a humanized monoclonal IgG4 antibody to α4 integrin, was evaluated in a randomized, controlled study to determine the ability of a six month regimen to maintain clinical response/remission achieved in natalizumab-treated subjects in the Phase III induction of response/remission study.

Methods

339 adult subjects with Crohn's disease (CD) who achieved response (≧70-point reduction in baseline Crohn's Disease Activity Index (CDAI)) and/or remission (CDAI<150) and had a CDAI score<220 after receiving three infusions of natalizumab in a first study were re-randomized in a 1:1 ratio to natalizumab (300 mg) (n=168) or placebo (n=171) for up to 12 additional monthly infusions. The primary endpoint was the proportion of subjects that did not lose that response from the first study at every time point for an additional 6 consecutive months in the second study. Loss of response was defined as a CDAI≧220 and ≧70-point increase from baseline CDAI in the second study or use of rescue intervention. Maintenance of remission was also assessed.

Results

At 6 months, 61% (103/168) of natalizumab-treated subjects (ITT population) continued to meet the criteria for clinical response versus 29% (49/170) of subjects re-randomized to receive placebo (p<0.001). 44% (57/130) in the natalizumab treatment group maintained clinical remission, compared with 26% (31/120) in the placebo group (p=0.003). In addition, 55% (37/67) of natalizumab-treated subjects taking steroids in the first study re-randomized to natalizumab in the second study were withdrawn from steroids, compared to 25% (19/76) re-randomized to placebo (p<0.001). No notable difference in the rates of serious and non-serious adverse events between treatment groups was observed.

In the second study, natalizumab demonstrated significant superiority over placebo in its ability to sustain response and remission at all consecutive time points over a 6-month period in the first study natalizumab-responders. Monthly administration of natalizumab for 6 months was well tolerated and enabled subjects to be successfully withdrawn from steroids.

### Example 4

Allograft Study 1

Transplantation

Allograft rejection is defined as the expression of an immune reaction of the recipient, against foreign antigens of the transplanted tissue. This reaction is mediated by T lymphocytes. Cell adhesion events are of critical importance in the T-cell recognition of alloantigens, the trafficking of recipient immune and inflammatory cells into the graft, and the execution of cell-mediated effector functions. Previous studies have demonstrated that α₄β₁ positive mononuclear cells infiltrate acutely rejecting organ allografts, and α₄β₁-VCAM-1 as well as α₄β₁ CS-1-fibronectin (FN) in vivo interactions play an important role in the immune cascade triggered by transplantation of MHC-incompatible tissues. In addition, the blockade of adhesion molecules with monoclonal antibodies has resulted in variable degrees of prolongation of graft survival, depending on the experimental model (Coito et al., 1998; Korom et al., 1998; Isobe et al., 1994; Orosz et al., Paul et al., 1996). This beneficial therapeutic effect correlates with decreased infiltration by α₄β₁ positive cells, deposition of CS1-FN, and endothelial staining for VCAM-1, as determined by immunohistology/RNAse protection assay.

To further evaluate the therapeutic potential of α₄β₁ targeted therapy in transplantation rejection, three studies were conducted in a well-defined rat cardiac transplant model. The following report summarizes the data across all three studies. In each study the efficacy of α₄β₁ compounds (N-pyridine-3-sulfonyl)-L-(5,5-dimethyl-thiaprolyl-L-4-(N,N-dimethylcarbamyloxy)phenylalanine isopropyl ester; N-(1-methylpyrazole-4-sulfonyl)-L-(5,5-dimethyl)thiaprolyl-L-(4-N,N-dimethylcarbamyloxy)phenylalanine isopropyl ester; and N-[N-(3-pyridinesulfonyl)-L-3,3-dimethyl-4-thiaprolyl]-0-[1-methylpiperazin-4-ylcarbonyl]-L-tyrosine isopropyl ester), were compared against the α₄β₁ antibody and vehicle controls.

Methods

Hearts from Lewis X Brown Norway F1 hybrids were transplanted to the abdominal great vessels of Lewis recipients using standard microvascular techniques. Both rat strains were male inbred rats, weighing 200-300 g. Food and water was available at all times except during surgical and dosing procedures. The day of transplantation was noted as day 0. Twelve hours following the last dose approximately 1 ml of blood was collected into lithium/heparin anti-coagulant tubes. If any graft was rejected prior to the last day of dosing, blood samples were taken 12 hours after dosing on the day that graft rejection was noted. All blood samples were centrifuged at 3000 rpm for 10 minutes and the plasma was harvested, frozen on dry ice and stored frozen at −20 C prior to analysis. Allograft survival in the rats was evaluated by daily palpation of the transplanted heart through the recipient's flank. Rejection was taken as the day of complete cessation of the graft's heart beat. Upon determination of rejection, the abdomen of the recipient was incised and retracted to confirm cessation of the heart beat in the transplanted heart. If cessation of the heart beat was not confirmed the abdomen was surgically closed, and the grafted heart was evaluated daily.

Results

Treatment with the vehicle, 0.9% saline, results in rejection of all grafts by day +9. As has been noted in the literature, the typical pattern of graft rejection in saline treated animals, begins around day +6, with the majority of grafts rejecting on day +7 and very few grafts surviving beyond day +9. The results of animals treated with saline, across three studies, agrees well with reports in the literature. Treatment with GG5/3, an antibody against α₄β₁, prolonged absolute cardiac allograft survival from day +9 to day +19. In comparison with GG5/3, treatment with compound N-(1-methylpyrazole-4-sulfonyl)-L-(5,5-dimethyl)thiaprolyl-L-(4-N,N-dimethylcarbamyloxy)phenylalanine isopropyl ester results in a similar survival rate (33-26%) on day +14, however there is enhancement in absolute graft prolongation. There was one incidence of graft survival out to day +37, FIG. 2. The vehicle, 10% ethanol in corn oil, had no significant effect on graft prolongation. The results with compound N-[N-(3-pyridinesulfonyl)-L-3,3-dimethyl-4-thiaprolyl]-0-[1-methylpiperazin-4-ylcarbonyl]-L-tyrosine isopropyl ester, FIG. 3, suggest that N-[N-(3-pyridinesulfonyl)-L-3,3-dimethyl-4-thiaprolyl]-0-[1-methylpiperazin-4-ylcarbonyl]-L-tyrosine isopropyl ester significantly improves acute graft survival as demonstrated by a 66% survival rate on day +14 and 33% survival rate out to day +30 with N-[N-(3-pyridinesulfonyl)-L-3,3-dimethyl-4-thiaprolyl]-0-[1-methylpiperazin-4-ylcarbonyl]-L-tyrosine isopropyl ester treatment. This survival rate far exceeds previous survival rates obtained with treatment of either GG5/3 or N-(1-methylpyrazole-4-sulfonyl)-L-(5,5-dimethyl)thiaprolyl-L-(4-N,N-dimethylcarbamyloxy)phenylalanine isopropyl ester. There is also enhancement in absolute graft prolongation as demonstrated by one incidence of graft survival out to day +41.

Allograft Study 2

### Methods

Cardiac allografts were established. Dosing of compounds began on day 0, prior to transplantation and continued through day +10. The compounds N-pyridine-3-sulfonyl)-L-(5,5-dimethyl-thiaprolyl-L-4-(N,N-dimethylcarbamyloxy)phenylalanine isopropyl ester and N-(1-methylpyrazole-4-sulfonyl)-L-(5,5-dimethyl)thiaprolyl-L-(4-N,N-dimethylcarbamyloxy)phenylalanine isopropyl ester, in 10% ethanol/corn oil, were administered at 30 mg/kg, as a subcutaneous (s.c.) dose, at 12 hour intervals. The vehicle control, 10% ethanol/corn oil, was administered s.c., every 12 hours, day 0 through day +10. The antibody, GG5/3 in 0.9% saline, was administered at 3 mg/kg, s.c., on day 0, +3, +7 and +10. All GG5/3 animals received twice daily s.c. injections of 0.9% saline, every 12 hours, throughout day 0 through day +10. The vehicle control, 0.9% saline, was administered daily, s.c., day 0 through day +10. All dosing solutions were formulated daily at a constant dose volume of 5 ml/kg. The site of injection was rotated at each injection through four possible sites: right and left fore flank, right and left rear flank. A total of 28 allografts, amongst 5 groups, were evaluated in the study. Group 1, consisted of 6 animals treated with the monoclonal antibody, GG5/3. Groups 2 and 3 consisted of 6 animals per group, in which compounds N-pyridine-3-sulfonyl)-L-(5,5-dimethyl-thiaprolyl-L-4-(N,N-dimethylcarbamyloxy)phenylalanine isopropyl ester and N-(1-methylpyrazole-4-sulfonyl)-L-(5,5-dimethyl)thiaprolyl-L-(4-N,N-dimethylcarbamyloxy)phenylalanine isopropyl ester, were administered respectively. Group 4 had 4 animals treated with 10% ethanol/corn oil. Group 5 had 4 animals treated with 0.9% saline. Twelve hours following the last dose approximately 1 ml of blood was collected into lithium/heparin anti-coagulant tubes. If any graft was rejected prior to the last day of dosing, blood samples were drawn 12 hours after dosing on the day that graft rejection was noted. All samples were centrifuged at 3000 rpm for 10 minutes and the plasma was harvested, frozen on dry ice and stored frozen at −20 C prior to analysis. Allograft survival was evaluated by daily palpation of the transplanted heart through the recipient's flank. Rejection was taken as the day of complete cessation of graft's heart beat. Upon determination of rejection, the abdomen of the recipient was incised to confirm cessation of the heart beat in the transplanted heart. If cessation of the graft's heart beat was not confirmed the abdomen was surgically closed, and the graft was evaluated daily.

Results

Table 20 summarizes the results. As was expected, grafts in the 0.9% saline treated animals all exhibited rejection by day +7. The majority of grafts in the 10% ethanol/corn oil treated animals exhibited rejection by day +8, with the remaining animal within that group rejecting its graft at day +11. The compound (N-pyridine-3-sulfonyl)-L-(5,5-dimethyl-thiaprolyl-L-4-(N,N-dimethylcarbamyloxy)phenylalanine isopropyl ester) prolonged graft survival for a short period (day +7 to day +12), with all the grafts rejecting by day +12. The animals treated with GG5/3 showed significant prolongation of graft survival. Fifty percent of the grafts in the GG5/3 group were still functioning on day +14. Grafts in animals treated with N-(1-methylpyrazole-4-sulfonyl)-L-(5,5-dimethyl)thiaprolyl-L-(4-N,N-dimethylcarbamyloxy)phenylalanine isopropyl ester also showed significant graft survival, resembling the results obtained from the GG5/3 group.

TABLE 20
Rat cardiac allograft study
Section 1a

| | Day | | | | | | | | | | | | | | | | | | |
|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 |
| GG5/3 | | | 6 | 6 | 6 | 6 | 6 | 6 | 6 | 5 | 4 | 4 | 4 | 3 | 3 | 3 | 3 | 0 | 0 |
| N-pyridine-3-sulfonyl)-L-(5,5- | | | 6 | 6 | 6 | 6 | 6 | 6 | 6 | 6 | 4 | 2 | 2 | 1 | 0 | 0 | 0 | 0 | 0 |
| dimethyl-thiaprolyl-L-4-(N,N- | | | | | | | | | | | | | | | | | | | |
| dimethylcarbamyloxy)phenyl- | | | | | | | | | | | | | | | | | | | |
| alanine isopropyl ester | | | | | | | | | | | | | | | | | | | |
| N-(1-methylpyrazole-4-sulfonyl)- | | | 6 | 6 | 6 | 6 | 6 | 6 | 6 | 6 | 6 | 6 | 5 | 3 | 3 | 2 | 1 | 1 | 0 |
| L-(5,5-dimethyl)thiaprolyl-L- | | | | | | | | | | | | | | | | | | | |
| (4-N,N-dimethyl- | | | | | | | | | | | | | | | | | | | |
| carbamyloxy)phenylalanine | | | | | | | | | | | | | | | | | | | |
| isopropyl ester | | | | | | | | | | | | | | | | | | | |
| 10% Et/CO | | | 4 | 4 | 4 | 4 | 4 | 4 | 3 | 2 | 1 | 1 | 1 | 0 | 0 | 0 | 0 | 1 | 0 |
| 0.9% Saline | | | 4 | 4 | 4 | 4 | 4 | 4 | 2 | 0 | 0 | 0 | 0 | 0 | 0 | 0 | 0 | 0 | 0 |

Section 1a: Daily tabulation of the number of surviving grafts within each group, with respect to time.

Section 1b

| | Day | | | | | | | | | | | | | | | | | | |
|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 |
| GG5/3 | | | 100 | 100 | 100 | 100 | 100 | 100 | 100 | 83 | 66 | 66 | 66 | 50 | 50 | 50 | 50 | 0 | 0 |
| N-pyridine-3-sulfonyl)-L-(5,5- | | | 100 | 100 | 100 | 100 | 100 | 100 | 100 | 100 | 66 | 50 | 50 | 16 | 0 | 0 | 0 | 0 | 0 |
| dimethyl-thiaprolyl-L-4-(N,N- | | | | | | | | | | | | | | | | | | | |
| dimethyl-carbamyloxy)phenyl- | | | | | | | | | | | | | | | | | | | |
| alanine isopropyl ester | | | | | | | | | | | | | | | | | | | |
| N-(1-methyl-pyrazole-4-sulfonyl)- | | | 100 | 100 | 100 | 100 | 100 | 100 | 100 | 100 | 100 | 100 | 83 | 50 | 50 | 33 | 16 | 16 | 0 |
| L-(5,5-dimethyl) thiaprolyl-L-(4- | | | | | | | | | | | | | | | | | | | |
| N,N-dimethylcarbamyloxy)phenyl- | | | | | | | | | | | | | | | | | | | |
| alanine isopropyl ester | | | | | | | | | | | | | | | | | | | |
| 10% Et/CO | | | 100 | 100 | 100 | 100 | 100 | 100 | 75 | 50 | 25 | 25 | 25 | 0 | 0 | 0 | 0 | 0 | 0 |
| 0.9% Saline | | | 100 | 100 | 100 | 100 | 100 | 100 | 50 | 0 | 0 | 0 | 0 | 0 | 0 | 0 | 0 | 0 | 0 |

Section 1b: Data normalized to group size and expressed as the percent of surviving grafts within a group, with respect to time.

Section 1c

| Group | n = | Dose, s.c. |
|---|---|---|
| GG5/3 | 6 | 3 mg/kg |
| N-pyridine-3-sulfonyl)-L-(5,5-dimethyl-thiaprolyl- | 6 | 30 mg/kg |
| L-4-(N,N-dimethylcarbamyloxy)phenylalanine | | |
| isopropyl ester | | |
| N-(1-methylpyrazole-4-sulfonyl)-L-(5,5- | 6 | 30 mg/kg |
| dimethyl)thiaprolyl-L-(4-N,N- | | |
| dimethylcarbamyloxy)phenylalanine isopropyl ester | | |
| 10% Et/CO | 4 | . . .** |
| 0.9% Saline | 4 | . . .** |

Section 1c: Group design and dosing protocol for allograft study CT001.

Allograft Study 3
Methods

Cardiac allografts were established. Dosing of compounds began on day −2, 2 days prior to transplantation and continued through day +10. Compound N-(1-methylpyrazole-4-sulfonyl)-L-(5,5-dimethyl)thiaprolyl-L-(4-N,N-dimethylcarbamyloxy)phenylalanine isopropyl ester, in 10% ethanol/corn oil, was administered at 30 mg/kg, as a s.c. dose, at 12 hour intervals. The vehicle control, 10% ethanol/corn oil, was administered s.c., every 12 hours, day −2 through day +10. The antibody, GG5/3 in 0.9% saline, was administered at 3 mg/kg, s.c., on day 0, +3, +7 and +10. All GG5/3 animals received twice daily s.c. injections of 0.9% saline, every 12 hours, throughout day −2 through day +10. The vehicle control, 0.9% saline, was administered daily, s.c., day −2 through day +10. All dosing solutions were formulated daily at a constant dose volume of 5 ml/kg. The site of injection was rotated at each injection through four possible sites: right and left fore flank, right and left rear flank. A total of 20 allografts, amongst 4 groups, were evaluated in the study. Group 1 had 4 animals treated with 0.9% saline. Group 2 consisted of 6 animals treated with the monoclonal antibody, GG5/3. Group 3 had 4 animals treated with 10% ethanol/corn oil. Group 4 consisted of 6 animals in which compound N-(1-methylpyrazole-4-sulfonyl)-L-(5,5-dimethyl)thiaprolyl-L-(4-N,N-dimethylcarbamyloxy)phenylalanine isopropyl ester was administered at 30 mg/kg, s.c., at 12 hour intervals.

Twelve hours following the last dose approximately 1 ml of blood was collected into lithium/heparin anti-coagulant tubes. If any graft was rejected prior to the last day of dosing, blood samples were taken 12 hours after dosing on the day that graft rejection was noted. All samples were centrifuged at 3000 rpm for 10 minutes and the plasma was harvested, frozen on dry ice and stored frozen at −20 C prior to analysis. Allograft survival was evaluated by daily palpation of the transplanted heart through the recipient's flank. Rejection was taken as the day of complete cessation of graft's heart beat. Upon determination of rejection, the abdomen of the recipient was incised to confirm cessation of the heart beat in the transplanted heart. If cessation of the graft's heart beat was not confirmed the abdomen was surgically closed, and the graft was evaluated daily.

Results

Table 21 summarizes the results. The vehicle controls, 0.9% saline and 10% ethanol/corn oil groups exhibited very similar rejection patterns, with rejection of all grafts occurring on day +9. Treatment with GG5/3 significantly prolonged graft acceptance out to day +18. Compound N-(1-methylpyrazole-4-sulfonyl)-L-(5,5-dimethyl)thiaprolyl-L-(4-N,N-dimethylcarbamyloxy)phenylalanine isopropyl ester exhibited a pattern of graft prolongation similar to GG5/3, with graft acceptance out to day +16.

TABLE 21
Rat cardiac allograft study.
Section 2a

| | Day | | | | | | | | | | | | | | | | | | | | | |
|---|---|---|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 | 17 | 18 | 19 |
| 0.9% Saline | | | 4 | 4 | 4 | 4 | 4 | 4 | 4 | 3 | 3 | 0 | 0 | 0 | 0 | 0 | 0 | 0 | 0 | 0 | 0 | 0 |
| GG5/3 | | | 6 | 6 | 6 | 6 | 4 | 4 | 4 | 4 | 4 | 4 | 4 | 4 | 1 | 1 | 1 | 1 | 1 | 1 | 1 | 0 |
| 10% Et/CO | | | 4 | 4 | 4 | 4 | 4 | 4 | 4 | 2 | 2 | 0 | 0 | 0 | 0 | 0 | 0 | 0 | 0 | 0 | 0 | 0 |
| N-(1- | | | 6 | 6 | 6 | 6 | 6 | 6 | 6 | 6 | 6 | 4 | 4 | 3 | 3 | 1 | 1 | 1 | 1 | 0 | 0 | 0 |
| methylpyrazole-4- | | | | | | | | | | | | | | | | | | | | | | |
| sulfonyl)-L-(5,5- | | | | | | | | | | | | | | | | | | | | | | |
| dimethyl)thiaprolyl- | | | | | | | | | | | | | | | | | | | | | | |
| L-(4-N,N- | | | | | | | | | | | | | | | | | | | | | | |
| dimethyl- | | | | | | | | | | | | | | | | | | | | | | |
| carbamyloxy)phenyl- | | | | | | | | | | | | | | | | | | | | | | |
| alanine isopropyl ester | | | | | | | | | | | | | | | | | | | | | | |

Section 2a: Daily tabulation of the number of surviving grafts within each group, with respect to time.

Section 2b

| | Day | | | | | | | | | | | | | | | | | | | | | |
|---|---|---|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 | 17 | 18 | 19 |
| 0.9% Saline | | | 100 | 100 | 100 | 100 | 100 | 100 | 100 | 75 | 75 | 0 | 0 | 0 | 0 | 0 | 0 | 0 | 0 | 0 | 0 | 0 |
| GG5/3 | | | 100 | 100 | 100 | 100 | 67 | 67 | 67 | 67 | 67 | 67 | 67 | 67 | 16 | 16 | 16 | 16 | 16 | 16 | 16 | 0 |
| 10% Et/CO | | | 100 | 100 | 100 | 100 | 100 | 100 | 100 | 50 | 50 | 0 | 0 | 0 | 0 | 0 | 0 | 0 | 0 | 0 | 0 | 0 |
| N-(1- | | | 100 | 100 | 100 | 100 | 100 | 100 | 100 | 100 | 100 | 67 | 67 | 50 | 50 | 16 | 16 | 16 | 16 | 0 | 0 | 0 |
| methylpyrazole-4- | | | | | | | | | | | | | | | | | | | | | | |
| sulfonyl)-L-(5,5- | | | | | | | | | | | | | | | | | | | | | | |
| dimethyl)thiaprolyl- | | | | | | | | | | | | | | | | | | | | | | |
| L-(4-N,N- | | | | | | | | | | | | | | | | | | | | | | |
| dimethyl- | | | | | | | | | | | | | | | | | | | | | | |
| carbamyloxy)phenyl- | | | | | | | | | | | | | | | | | | | | | | |
| alanine isopropyl ester | | | | | | | | | | | | | | | | | | | | | | |

Section 2b: Data normalized to group size, and expressed as the percent of surviving grafts within a group, with respect to time.

Section 2c

| Group | n = | Dose, s.c. |
|---|---|---|
| 0.9% Saline | 4 | ** |
| GG5/3 | 6 | 3 mg/kg |
| 10% Et/CO | 4 | ** |
| N-(1-methylpyrazole-4-sulfonyl)-L-(5,5- | 6 | 30 mg/kg |
| dimethyl)thiaprolyl-L-(4-N,N- | | |
| dimethylcarbamyloxy)phenylalanine isopropyl ester | | |

Section 2c: Group design and dosing protocol for allograft study CT002.

Allograft Study 4
Methods

Cardiac allografts were established. Dosing of compounds began on day −2, 2 days prior to transplantation and continued through day +10. Compound N-[N-(3-pyridinesulfonyl)-L-3,3-dimethyl-4-thiaprolyl]-0-[1-methylpiperazin-4-ylcarbonyl]-L-tyrosine isopropyl ester, in 0.9% saline, was administered at 30 mg/kg, as a s.c. dose, at 12 hour intervals. The vehicle control, 0.9% saline, pH adjusted to a value of 5, to match the dosing solution containing the N-[N-(3-pyridinesulfonyl)-L-3,3-dimethyl-4-thiaprolyl]-0-[1-methylpiperazin-4-ylcarbonyl]-L-tyrosine isopropyl ester, was administered via the same route, i.e. every 12 hours, day −2 through day +10. The antibody, GG5/3 in 0.9% saline, was administered at 3 mg/kg, s.c., on day 0, +3, +7 and +10. All GG5/3 animals received twice daily s.c. injections of 0.9% saline, every 12 hours, from day −2 through day +10. The vehicle control, 0.9% saline, was administered daily, s.c., day 0 through day +10. All dosing solutions were formulated daily at a constant dose volume of 5 ml/kg. The site of injection was rotated at each injection through four possible sites: right and left fore flank, right and left rear flank. A total of 21 allografts, amongst 5 groups, were evaluated in the study. Group 1 had 4 animals treated with 0.9% saline. Two animals within the 0.9% saline group received saline adjusted to a pH a value of 5. (The pH value of the N-[N-(3-pyridinesulfonyl)-L-3,3-dimethyl-4-thiaprolyl]-0-[1-methylpiperazin-4-ylcarbonyl]-L-tyrosine isopropyl ester dosing solution.) The remaining two animals received saline of pH 7. Group 2 consisted of 6 animals treated with the monoclonal antibody, GG5/3. Group 3 had 2 animals treated with 10% ethanol/corn oil. Group 4 consisted of 3 animals in which compound AN N-(1-methylpyrazole-4-sulfonyl)-L-(5,5-dimethyl)thiaprolyl-L-(4-N,N-dimethylcarbamyloxy)phenylalanine isopropyl ester was administered at 30 mg/kg, s.c., at 12 hour intervals. Group5 consisted of 6 animals, dosed with compound N-[N-(3-pyridinesulfonyl)-L-3,3-dimethyl-4-thiaprolyl]-0-[1-methylpiperazin-4-ylcarbonyl]-L-tyrosine isopropyl ester 2 at 30 mg/kg, s.c., at 12 hour intervals. Twelve hours following the last dose approximately 1 ml of blood was collected into lithium/heparin anti-coagulant tubes. If any graft was rejected prior to the last day of dosing, blood samples were taken 12 hours after dosing on the day that graft rejection was noted. All samples were centrifuged at 3000 rpm for 10 minutes and the plasma was harvested, frozen on dry ice and stored frozen at −20 C prior to dispatch for analysis. Allograft survival in the rats was evaluated by daily palpation of the transplanted heart through the recipient's flank. Rejection was taken as the day of complete cessation of graft's heart beat. Upon determination of rejection, the abdomen of the recipient was incised to confirm cessation of the heart beat in the transplanted heart. If cessation of the heart beat was not confirmed the abdomen was surgically closed, and the graft was evaluated daily.

Results

Table 22 summarizes the results. The was no difference in graft rejection within the saline group with respect to pH. Saline treated animals exhibited graft rejection on day +7, as expected. The 10% ethanol/corn oil group exhibited a very similar pattern graft rejection to that of the saline group, with rejection of all the grafts occurring on day +9. Treatment with GG5/3 significantly prolonged graft acceptance, with complete graft rejection occurring on day +17. Compounds N-(1-methylpyrazole-4-sulfonyl)-L-(5,5-dimethyl)thiaprolyl-L-(4-N,N-dimethylcarbamyloxy)phenylalanine isopropyl ester and N-[N-(3-pyridinesulfonyl)-L-3,3-dimethyl-4-thiaprolyl]-0-[1-methylpiperazin-4-ylcarbonyl]-L-tyrosine isopropyl ester significantly prolong allograft acceptance beyond the level obtain with GG5/3 in this experiment. Fifty percent or greater survival rate was noted on day +19 in both N-(1-methylpyrazole-4-sulfonyl)-L-(5,5-dimethyl)thiaprolyl-L-(4-N,N-dimethylcarbamyloxy)phenylalanine isopropyl ester and N-[N-(3-pyridinesulfonyl)-L-3,3-dimethyl-4-thiaprolyl]-0-[1-methylpiperazin-4-ylcarbonyl]-L-tyrosine isopropyl ester groups. One graft in the N-(1-methylpyrazole-4-sulfonyl)-L-(5,5-dimethyl)thiaprolyl-L-(4-N,N-dimethylcarbamyloxy)phenylalanine isopropyl ester survived out to day +37 and one graft in the N-[N-(3-pyridinesulfonyl)-L-3,3-dimethyl-4-thiaprolyl]-0-[1-methylpiperazin-4-ylcarbonyl]-L-tyrosine isopropyl ester survived out to day +41.

TABLE 22
Rat cardiac allograft

| | Day | | | | | | | | | | | | | | | | | | | | | |
|---|---|---|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 | 17 | 18 | 19 |
| 0.9% Saline | | | 4 | 4 | 4 | 4 | 4 | 4 | 2 | 0 | 0 | 0 | 0 | 0 | 0 | 0 | 0 | 0 | 0 | 0 | 0 | 0 |
| GG5/3 | | | 6 | 6 | 6 | 6 | 6 | 6 | 6 | 5 | 5 | 4 | 4 | 4 | 3 | 2 | 1 | 1 | 1 | 0 | 0 | 0 |
| 10%/Et/CO | | | 2 | 2 | 2 | 2 | 2 | 2 | 2 | 1 | 1 | 0 | 0 | 0 | 0 | 0 | 0 | 0 | 0 | 0 | 0 | 0 |
| N-1(1-methylpyrazaole-4-sulfonyl)-L- | | | 3 | 3 | 3 | 3 | 3 | 3 | 3 | 3 | 2 | 2 | 2 | 2 | 2 | 2 | 2 | 2 | 2 | 2 | 2 | 2 |
| (5,5- | | | | | | | | | | | | | | | | | | | | | | |
| dimethylcarbamyloxy)phenylalanine | | | | | | | | | | | | | | | | | | | | | | |
| isopropyl ester | | | | | | | | | | | | | | | | | | | | | | |
| N-[N-(3-pyridinesulfonyl)-L-3,3- | | | 6 | 6 | 6 | 6 | 6 | 6 | 6 | 6 | 6 | 5 | 4 | 4 | 4 | 4 | 4 | 4 | 3 | 3 | 3 | 3 |
| dimehtyl-4-thiaprolyl]-0-[1-methylpiper | | | | | | | | | | | | | | | | | | | | | | |
| azin-4-ylcarbonyl]-1-tyrosine isopropyl | | | | | | | | | | | | | | | | | | | | | | |
| ester | | | | | | | | | | | | | | | | | | | | | | |

| | Day | | | | | | | | | | | | | | | | | | | | | | |
|---|---|---|---|---|---|---|---|---|---|---|---|---|---|---|---|---|---|---|---|---|---|---|---|
| | 20 | 21 | 22 | 23 | 24 | 25 | 26 | 27 | 28 | 29 | 30 | 31 | 32 | 33 | 34 | 35 | 36 | 37 | 38 | 39 | 40 | 41 | 42 |
| 0.9% Saline | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| GG5/3 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 10%/Et/CO | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| N-1(1-methylpyrazaole-4-sulfonyl)- | 2 | 2 | 2 | 2 | 2 | 2 | 2 | 2 | 1 | 1 | 1 | 1 | 1 | 1 | 1 | 1 | 1 | 1 | 0 | 0 | 0 | 0 | 0 |
| L-(5,5- | | | | | | | | | | | | | | | | | | | | | | | |
| dimethylcarbamyloxy)phenylalanine | | | | | | | | | | | | | | | | | | | | | | | |
| isopropyl ester | | | | | | | | | | | | | | | | | | | | | | | |
| N-[N-(3-pyridinesulfonyl)-L-3,3- | 2 | 2 | 2 | 2 | 2 | 2 | 2 | 2 | 2 | 2 | 2 | 1 | 1 | 1 | 1 | 1 | 1 | 1 | 1 | 1 | 1 | 1 | 0 |
| dimehtyl-4-thiaprolyl]-0-[1- | | | | | | | | | | | | | | | | | | | | | | | |
| methylpiper azin-4-ylcarbonyl]-1- | | | | | | | | | | | | | | | | | | | | | | | |
| tyrosine isopropyl ester | | | | | | | | | | | | | | | | | | | | | | | |

Section 3b

| | Day | | | | | | | | | | | | | | | | | | | | | |
|---|---|---|---|---|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 | 15 | 16 | 17 | 18 | 19 |
| 0.9% Saline | | | 100 | 100 | 100 | 100 | 100 | 100 | 50 | 0 | 0 | 0 | 0 | 0 | 0 | 0 | 0 | 0 | 0 | 0 | 0 | 0 |
| GG5/3 | | | 100 | 100 | 100 | 100 | 100 | 100 | 100 | 83 | 83 | 66 | 66 | 66 | 50 | 33 | 16 | 16 | 16 | 0 | 0 | 0 |
| 10%/Et/CO | | | 100 | 100 | 100 | 100 | 100 | 100 | 100 | 50 | 50 | 0 | 0 | 0 | 0 | 0 | 0 | 0 | 0 | 0 | 0 | 0 |
| N-1(1-methyl- | | | 100 | 100 | 100 | 100 | 100 | 100 | 100 | 100 | 66 | 66 | 66 | 66 | 66 | 66 | 66 | 66 | 66 | 66 | 66 | 66 |
| pyrazaole-4- | | | | | | | | | | | | | | | | | | | | | | |
| sulfonyl)-L-(5,5- | | | | | | | | | | | | | | | | | | | | | | |
| dimethyl- | | | | | | | | | | | | | | | | | | | | | | |
| carbamyloxy)phenyl- | | | | | | | | | | | | | | | | | | | | | | |
| alanine isopropyl ester | | | | | | | | | | | | | | | | | | | | | | |
| N-[N-(3- | | | 100 | 100 | 100 | 100 | 100 | 100 | 100 | 100 | 100 | 83 | 66 | 66 | 66 | 66 | 66 | 66 | 50 | 50 | 50 | 50 |
| pyridinesulfonyl)-L- | | | | | | | | | | | | | | | | | | | | | | |
| 3,3-dimehtyl-4- | | | | | | | | | | | | | | | | | | | | | | |
| thiaprolyl]-0-[1- | | | | | | | | | | | | | | | | | | | | | | |
| methylpiper azin-4- | | | | | | | | | | | | | | | | | | | | | | |
| ylcarbonyl]-1-tyrosine | | | | | | | | | | | | | | | | | | | | | | |
| isopropyl ester | | | | | | | | | | | | | | | | | | | | | | |

| | Day | | | | | | | | | | | | | | | | | | | | | | |
|---|---|---|---|---|---|---|---|---|---|---|---|---|---|---|---|---|---|---|---|---|---|---|---|
| | 20 | 21 | 22 | 23 | 24 | 25 | 26 | 27 | 28 | 29 | 30 | 31 | 32 | 33 | 34 | 35 | 36 | 37 | 38 | 39 | 40 | 41 | 42 |
| 0.9% Saline | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| GG5/3 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 10%/Et/CO | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| N-1(1-methylpyrazaole-4- | 66 | 66 | 66 | 66 | 66 | 66 | 66 | 66 | 33 | 33 | 33 | 33 | 33 | 33 | 33 | 33 | 33 | 33 | 0 | 0 | 0 | 0 | 0 |
| sulfonyl)-L-(5,5- | | | | | | | | | | | | | | | | | | | | | | | |
| dimethylcarbamyloxy)phenylalanine | | | | | | | | | | | | | | | | | | | | | | | |
| isopropyl ester | | | | | | | | | | | | | | | | | | | | | | | |
| N-[N-(3-pyridinesulfonyl)- | 33 | 33 | 33 | 33 | 33 | 33 | 33 | 33 | 33 | 33 | 33 | 16 | 16 | 16 | 16 | 16 | 16 | 16 | 16 | 16 | 16 | 16 | 0 |
| L-3,3-dimehtyl-4- | | | | | | | | | | | | | | | | | | | | | | | |
| thiaprolyl]-0-[1- | | | | | | | | | | | | | | | | | | | | | | | |
| methylpiper azin-4- | | | | | | | | | | | | | | | | | | | | | | | |
| ylcarbonyl]-1-tyrosine | | | | | | | | | | | | | | | | | | | | | | | |
| isopropyl ester | | | | | | | | | | | | | | | | | | | | | | | |

Section 3b: Data normalized to group size and expression as the percent of surviving grafts within a group, with respect to time.

| Group | N = | Dose, s.c. |
|---|---|---|
| 0.9% Saline | 4 | ** |
| GG5/3 | 6 | 3 mg/kg |
| 10% ET/CO | 2 | ** |
| N-1(1-methylpyrazaole-4-sulfonyl)-L-(5,5- | 3 | 30 mg/kg |
| dimethylcarbamyloxy)phenylalanine | | |
| isopropyl ester | | |
| N-[N-(3-pyridinesulfonyl)-L-3,3-dimehtyl- | 6 | 30 mg/kg |
| 4-thiaprolyl]-0-[1-methylpiper azin-4- | | |
| ylcarbonyl]-1-tyrosine isopropyl ester | | |

Section 3c: Group design and dosing protocol for allograft study CT003.

Allograft Study 5
Methods

Cardiac allografts were established. Dosing of compounds began on day −2, 2 days prior to transplantation and continued through day +10. Compound N-[N-(3-pyridinesulfonyl)-L-3,3-dimethyl-4-thiaprolyl]-0-[1-methylpiperazin-4-ylcarbonyl]-L-tyrosine isopropyl ester, in 0.9% saline, was administered at 1 of 3 doses, 30 mg/kg, 10 mg/kg or 3 mg/kg, as a s.c. dose, at 12 hour intervals. The vehicle control, 0.9% saline, pH adjusted to a value of 5, to match the dosing solution containing the N-[N-(3-pyridinesulfonyl)-L-3,3-dimethyl-4-thiaprolyl]-0-[1-methylpiperazin-4-ylcarbonyl]-L-tyrosine isopropyl ester, ws administered via the same route, i.e. every 12 hours, day −2 through day +10. All dosing solutions were formulated daily at a constant dose volume of 5 ml/kg. The site of injection was rotated at each injection through four possible sites: right and left fore flank, right and left rear flank. A total of 20 allografts, amongst 4 groups, were evaluated in the study. Group 1 had 3 animals treated with 0.9% saline. Group 2 consisted of 6 animals treated with 30 mg/kg N-[N-(3-pyridinesulfonyl)-L-3,3-dimethyl-4-thiaprolyl]-0-[1-methylpiperazin-4-ylcarbonyl]-L-tyrosine isopropyl ester. Group 3 had 5 animals treated with 10 mg/kg N-[N-(3-pyridinesulfonyl)-L-3,3-dimethyl-4-thiaprolyl]-0-[1-methylpiperazin-4-ylcarbonyl]-L-tyrosine isopropyl ester. Group 4 consisted of 5 animals treated with 3 mg/kg N-[N-(3-pyridinesulfonyl)-L-3,3-dimethyl-4-thiaprolyl]-0-[1-methylpiperazin-4-ylcarbonyl]-L-tyrosine isopropyl ester. Twelve hours following the last dose approximately 1 ml of blood was collected into lithium/heparin anti-coagulant tubes. If any graft was rejected prior to the last day of dosing, blood samples were taken 12 hours after dosing on the day that graft rejection was noted. All samples were centrifuged at 3000 rpm for 10 minutes and the plasma was harvested, frozen on dry ice and stored frozen at −20 C prior to dispatch for analysis. Allograft survival in the rats was evaluated by daily palpation of the transplanted heart through the recipient's flank. Rejection was taken as the day of complete cessation of graft's heart beat. Upon determination of rejection, the abdomen of the recipient was incised to confirm cessation of the heart beat in the transplanted heart. If cessation of the heart beat was not confirmed the abdomen was surgically closed, and the graft was evaluated daily.

Results

Table 23 summarizes the results. Saline treated animals exhibited graft rejection by day +9, as expected. The 30 mg/kg N-[N-(3-pyridinesulfonyl)-L-3,3-dimethyl-4-thiaprolyl]-0-[1-methylpiperazin-4-ylcarbonyl]-L-tyrosine isopropyl ester treatment group exhibits prolonged graft acceptance. As the study is still in progress, the latest date of graft survival is out to day +22, with a survival rate of 83%. At a midrange dose of 10 mg/kg of N-[N-(3-pyridinesulfonyl)-L-3,3-dimethyl-4-thiaprolyl]-0-[1-methylpiperazin-4-ylcarbonyl]-L-tyrosine isopropyl ester, the survival rate has decreased. Rejection at this dose begins on day+8 and the survival rate steady drops, reaching 20% by day +22. The lowest dose of N-[N-(3-pyridinesulfonyl)-L-3,3-dimethyl-4-thiaprolyl]-0-[1-methylpiperazin-4-ylcarbonyl]-L-tyrosine isopropyl ester, 3 mg/kg, did not provide any protection against graft rejection. Rejection at the 3 mg/kg dose begins on day+7 and is completed by day+10. This pattern of accelerated rejection does not significantly differ from the vehicle control, 0.9% saline.

TABLE 23
Rat cardiac allograft study.

| | Day | | | | | | | | | | | | | | | | |
|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 |
| 0.9% Saline | | | 3 | 3 | 3 | 3 | 3 | 3 | 3 | 3 | 2 | 0 | 0 | 0 | 0 | 0 | 0 |
| N-[N-(3-pyridinesulfonyl)-L-3,3-dimethyl-4- | | | 6 | 6 | 6 | 6 | 6 | 6 | 6 | 6 | 6 | 6 | 6 | 6 | 6 | 6 | 6 |
| thiaprolyl]-0-[1-methylpiperazine-4-ylcarbonyl]- | | | | | | | | | | | | | | | | | |
| L]tyrosine isopropyl ester @ 30 mg/kg | | | | | | | | | | | | | | | | | |
| N-[N-(3-pyridinesulfonyl)-L-3,3-dimethyl-4- | | | 5 | 5 | 5 | 5 | 5 | 5 | 5 | 5 | 5 | 4 | 4 | 4 | 4 | 2 | 2 |
| thiaprolyl]-0-[1-methylpiperazine-4-ylcarbonyl]-L- | | | | | | | | | | | | | | | | | |
| tyrosine isopropyl ester @10 mg/kg | | | | | | | | | | | | | | | | | |
| N-[N-(3-pyridinesulfinyl)-L-3,3-dimethyl-4- | | | 6 | 6 | 6 | 6 | 6 | 6 | 6 | 5 | 5 | 3 | 0 | 0 | 0 | 0 | 0 |
| thiaprolyl]-0-[1-methylpiperazin-4-ylcarbonyl]-L- | | | | | | | | | | | | | | | | | |
| tyrosine isopropyl ester @3 mg/kg | | | | | | | | | | | | | | | | | |
| | Day | | | | | | | | | | | | | | | | |
| | 15 | 16 | 17 | 18 | 19 | 20 | 21 | 22 | 23 | 24 | 25 | 26 | 27 | 28 | 29 | 30 | 31 |
| 0.9% Saline | 0 | 0 | 0 | 0 | 0 | 0 | 0 | P | | | | | | | | | |
| N-[N-(3-pyridinesulfonyl)-L-3,3-dimethyl-4- | 6 | 6 | 6 | 6 | 6 | 6 | 5 | P | | | | | | | | | |
| thiaprolyl]-0-[1-methylpiperazine-4-ylcarbonyl]- | | | | | | | | | | | | | | | | | |
| L]tyrosine isopropyl ester @ | | | | | | | | | | | | | | | | | |
| 30 mg/kg | | | | | | | | | | | | | | | | | |
| N-[N-(3-pyridinesulfonyl)-L-3,3-dimethyl-4- | 2 | 1 | 1 | 1 | 1 | 1 | 1 | P | | | | | | | | | |
| thiaprolyl]-0-[1-methylpiperazine-4-ylcarbonyl]-L- | | | | | | | | | | | | | | | | | |
| tyrosine isopropyl ester @ | | | | | | | | | | | | | | | | | |
| 10 mg/kg | | | | | | | | | | | | | | | | | |
| N-[N-(3-pyridinesulfinyl)-L-3,3-dimethyl-4- | 0 | 0 | 0 | 0 | 0 | 0 | 0 | P | | | | | | | | | |
| thiaprolyl]-0-[1-methylpiperazin-4-ylcarbonyl]- | | | | | | | | | | | | | | | | | |
| L-tyrosine isopropyl ester @3 mg/kg | | | | | | | | | | | | | | | | | |

Section 4b: Data normalized to group size.

| | Day | | | | | | | | | | | | | | | | |
|---|---|---|---|---|---|---|---|---|---|---|---|---|---|---|---|---|---|
| | −2 | −1 | 0 | 1 | 2 | 3 | 4 | 5 | 6 | 7 | 8 | 9 | 10 | 11 | 12 | 13 | 14 |
| 0.9% Saline | | | | 100 | 100 | 100 | 100 | 100 | 100 | 100 | 67 | 0 | 0 | 0 | 0 | 0 | 0 |
| N-[N-(3-pyridine-sulfonyl)-L-3,3- | | | | 100 | 100 | 100 | 100 | 100 | 100 | 100 | 100 | 100 | 100 | 100 | 100 | 100 | 100 |
| dimethyl-4-thiaprolyl]-0-[1- | | | | | | | | | | | | | | | | | |
| methylpiperazine-4-ylcarbonyl]-L] | | | | | | | | | | | | | | | | | |
| tyrosine isopropyl ester @ | | | | | | | | | | | | | | | | | |
| 30 mg/kg | | | | | | | | | | | | | | | | | |
| N-[N-(3-pyridine-sulfonyl)-L-3,3- | | | | 100 | 100 | 100 | 100 | 100 | 100 | 100 | 100 | 80 | 80 | 80 | 80 | 40 | 40 |
| dimethyl-4-thiaprolyl]-0-[1- | | | | | | | | | | | | | | | | | |
| methylpiperazine-4-ylcarbonyl]-L- | | | | | | | | | | | | | | | | | |
| tyrosine isopropyl ester @ | | | | | | | | | | | | | | | | | |
| 10 mg/kg | | | | | | | | | | | | | | | | | |
| N-[N-(3-pyridine-sulfinyl)-L-3,3- | | | | 100 | 100 | 100 | 100 | 100 | 100 | 83 | 83 | 50 | 0 | 0 | 0 | 0 | 0 |
| dimethyl-4-thiaprolyl]-0-[1- | | | | | | | | | | | | | | | | | |
| methylpiperazin-4-ylcarbonyl]-L- | | | | | | | | | | | | | | | | | |
| tyrosine | | | | | | | | | | | | | | | | | |
| isopropyl ester @3 mg/kg | | | | | | | | | | | | | | | | | |

| | Day | | | | | | | | | | | | | | | | |
|---|---|---|---|---|---|---|---|---|---|---|---|---|---|---|---|---|---|
| | 15 | 16 | 17 | 18 | 19 | 20 | 21 | 22 | 23 | 24 | 25 | 26 | 27 | 28 | 29 | 30 | 31 |
| 0.9% Saline | 0 | 0 | 0 | 0 | 0 | 0 | 0 | P | | | | | | | | | |
| N-[N-(3-pyridinesulfonyl)-L-3,3- | 100 | 100 | 100 | 100 | 100 | 100 | 83 | P | | | | | | | | | |
| dimethyl-4-thiaprolyl]-0-[1- | | | | | | | | | | | | | | | | | |
| methylpiperazine-4-ylcarbonyl]- | | | | | | | | | | | | | | | | | |
| L]tyrosine isopropyl ester @ 30 mg/kg | | | | | | | | | | | | | | | | | |
| N-[N-(3-pyridinesulfonyl)-L-3,3- | 40 | 20 | 20 | 20 | 20 | 20 | 20 | P | | | | | | | | | |
| dimethyl-4-thiaprolyl]-0-[1- | | | | | | | | | | | | | | | | | |
| methylpiperazine-4-ylcarbonyl]-L- | | | | | | | | | | | | | | | | | |
| tyrosine isopropyl ester @10 mg/kg | | | | | | | | | | | | | | | | | |
| N-[N-(3-pyridinesulfinyl)-L-3,3- | 0 | 16 | 17 | 18 | 19 | 20 | 21 | 22 | | | | | | | | | |
| dimethyl-4-thiaprolyl]-0-[1- | | | | | | | | | | | | | | | | | |
| methylpiperazin-4-ylcarbonyl]-L-tyrosine | | | | | | | | | | | | | | | | | |
| isopropyl ester @3 mg/kg | | | | | | | | | | | | | | | | | |

| Group | N = | Dose, s.c. |
|---|---|---|
| 0.9% Saline | 3 | ** |
| N-[N(3-pyridinesulfonyl)-:-3,3-dimethyl-4-thiaprolyl]-0- | 6 | 30 mg/kg |
| [1-methylpiperazine-4-ylcarbonyl]-L-tyrosine isopropyl ester @30 mg/kg | | |
| N-[N(3-pyridinesulfonyl)-L-3,3-dimethyl-4-thiaprolyl]-0- | 5 | 10 mg/kg |
| [1-methylpiperazin-4-ylcarbonyl]-L-tyrosine isopropyl ester @10 mg/kg | | |
| N-[N-(3-pyridinesulfonyl)-L-3,3-dimethyl-4-thiaprolyl]-0- | 6 | 3 mg/kg |
| [1-methylpiperazin-4-ylcarbonyl]-L-tyrosine isopropyl ester @3 mg/kg | | |

All of the above publications, patents and patent applications are herein incorporated by reference in their entirety to the same extent as if each individual publication, patent or patent application was specifically and individually indicated to be incorporated by reference in its entirety.

While the invention has been described in detail and with reference to specific embodiments thereof, it will be apparent to one skilled in the art that various changes and modifications can be made without departing from the spirit and scope thereof.

What is claimed is:

1. A method of reducing and/or eliminating a need for steroid treatment in a subject with a disease selected from the group consisting of inflammatory bowel disease, asthma, multiple schlerosis, rheumatoid arthritis, graft vs. host disease, host vs. graft disease, and spondyloarthropathies and combinations thereof comprising:
administering to the subject in need thereof a steroid sparing agent in a steroid sparing effective amount.

2. The method of claim 1, wherein the steroid sparing agent is a compound of formula I: 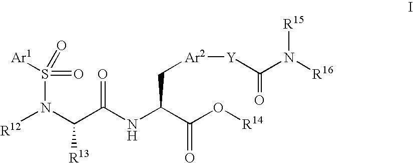
wherein:
Ar¹ is selected from the group consisting of aryl, substituted aryl, heteroaryl, and substituted heteroaryl;
Ar² is selected from the group consisting of aryl, substituted aryl, heteroaryl and substituted heteroaryl;
R¹² and R¹³ together with the nitrogen atom bound to R¹² and the carbon atom bound to R¹³ form a heterocyclic or substituted heterocyclic group;
R¹⁴ is selected from the group consisting of hydrogen, alkyl, substituted alkyl, cycloalkyl, substituted cycloalkyl, aryl, and substituted aryl;
R¹⁵ is selected from the group consisting of alkyl, and substituted alkyl, or R¹⁵ and R¹⁶ together with the nitrogen atom to which they are bound form a heterocyclic or substituted heterocyclic group;
R¹⁶ is selected from the group consisting of alkyl and substituted alkyl or R¹⁵ and R¹⁶ together with the nitrogen atom to which they are bound form a heterocyclic or substituted heterocyclic group; and
Y is selected from the group consisting of —O— and —NR¹⁰⁰—, wherein R¹⁰⁰ is hydrogen or alkyl;
and pharmaceutically acceptable salts thereof.

3. The method of claim 2, wherein the steroid sparing agent is a compound of formula Ia: 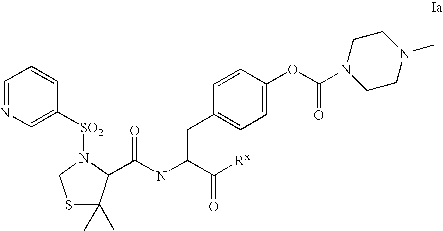
wherein R^{x} is hydroxy or C₁₋₅ alkoxy and pharmaceutically acceptable salts thereof.

4. The method of claim 1, wherein the steroid sparing compound is a compound of formula II: 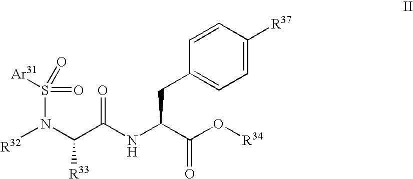
wherein:
Ar³¹ is selected from the group consisting of aryl, substituted aryl, heteroaryl, and substituted heteroaryl;
R³² and R³³ together with the nitrogen atom bound to R³² and the carbon atom bound to R³³ form a heterocyclic or substituted heterocyclic group;
R³⁴ is selected from the group consisting of hydrogen, alkyl, substituted alkyl, cycloalkyl, substituted cycloalkyl, aryl, and substituted aryl; and
R³⁷ is aryl, heteroaryl, substituted aryl, substituted heteroaryl, heterocyclic, substituted heterocyclic, aryloxy, substituted aryloxy, aralkoxy, substituted aralkoxy, heteroaryloxy, substituted heteroaryloxy;
and pharmaceutically acceptable salts thereof.

5. The method of claim 1, wherein the steroid sparing compound is a compound of formula IIIa or formula IIIb: 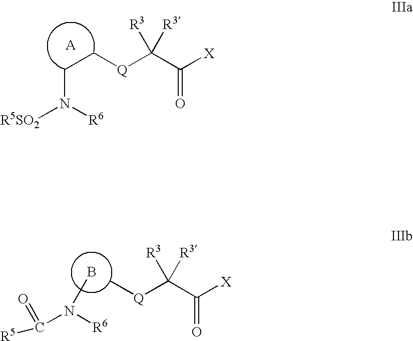
wherein:
R³ and R^{3′} are independently selected from the group consisting of hydrogen, isopropyl, —CH₂Z where Z is selected from the group consisting of hydrogen, hydroxyl, acylamino, alkyl, alkoxy, aryloxy, aryl, aryloxyaryl, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted alkyl, substituted alkoxy, substituted aryl, substituted aryloxy, substituted aryloxyaryl, substituted cycloalkyl, heteroaryl, substituted heteroaryl, heterocyclic and substituted heterocyclic, or
R³ and R^{3′} are joined to form a substituent selected from the group consisting of ═CHZ where Z is defined above provided that Z is not hydroxyl or thiol, cycloalkyl, substituted cycloalkyl, cycloalkenyl, substituted cycloalkenyl, heterocyclic and substituted heterocyclic;
X is selected from the group consisting of hydroxyl, alkoxy, substituted alkoxy, alkenoxy, substituted alkenoxy, cycloalkoxy, substituted cycloalkoxy, cycloalkenoxy, substituted cycloalkenoxy, aryloxy, substituted aryloxy, heteroaryloxy, substituted heteroaryloxy, heterocyclyloxy, substituted heterocyclyloxy and —NR″R″ where each R″ is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic and substituted heterocyclic;
Q is selected from the group consisting of —O—, —S—, —S(O)—, —S(O)₂—, and —NR⁴—;
ring A and ring B independently form a heteroaryl or substituted heteroaryl group having two nitrogen atoms in the heteroaryl ring;
R⁴ is selected from the group consisting of hydrogen, alkyl, substituted alkyl, alkenyl, substituted alkenyl, cycloalkyl, substituted cycloalkyl, cycloalkenyl, substituted cycloalkenyl, aryl, substituted aryl, heteroaryl, substituted heteroaryl, heterocyclic and substituted heterocyclic;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, and substituted heteroaryl;
or optionally, one of, R⁴ and ring A, R⁴ and R⁵, R⁴ and R⁶, or R⁵ and R⁶, together with the atoms to which they are bound, can be joined to form a heterocyclic or substituted heterocyclic ring;
provided that ring B does not form a 6-amino or substituted amino pyrimidin-4-yl group;
and enantiomers, diastereomers and pharmaceutically acceptable salts thereof.

6. The method of claim 1, wherein the steroid sparing compound is a compound of formula IVa, IVb, IVc, or IVd: 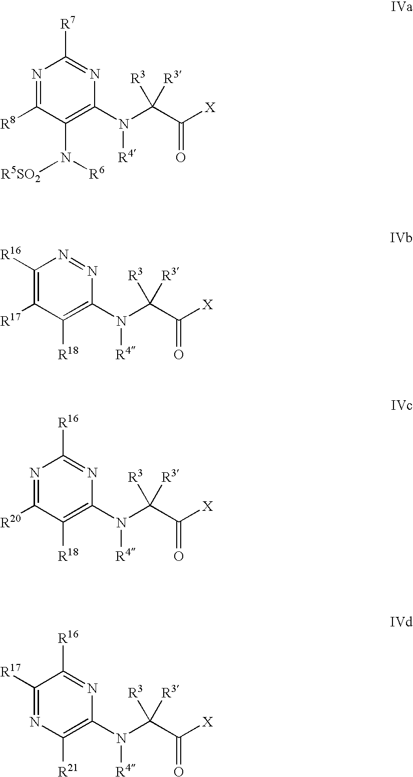
wherein:
R³ and R³ are independently selected from the group consisting of hydrogen, isopropyl, —CH₂Z where Z is selected from the group consisting of hydrogen, hydroxyl, acylamino, alkyl, alkoxy, aryloxy, aryl, aryloxyaryl, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted alkyl, substituted alkoxy, substituted aryl, substituted aryloxy, substituted aryloxyaryl, substituted cycloalkyl, heteroaryl, substituted heteroaryl, heterocyclic and substituted heterocyclic, or
R³ and R^{3′} are joined to form a substituent selected from the group consisting of ═CHZ where Z is defined above provided that Z is not hydroxyl or thiol, cycloalkyl, substituted cycloalkyl, cycloalkenyl, substituted cycloalkenyl, heterocyclic and substituted heterocyclic;
X is selected from the group consisting of hydroxyl, alkoxy, substituted alkoxy, alkenoxy, substituted alkenoxy, cycloalkoxy, substituted cycloalkoxy, cycloalkenoxy, substituted cycloalkenoxy, aryloxy, substituted aryloxy, heteroaryloxy, substituted heteroaryloxy, heterocyclyloxy, substituted heterocyclyloxy and —NR″R″ where each R″ is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic and substituted heterocyclic;
R^{4′} is selected from the group consisting of hydrogen and alkyl or, optionally, one of, R^{4′} and R⁵, R⁴′ and R⁶, R⁵ and R⁶, R⁵ and R⁸, or R⁶ and R⁸, together with the atoms to which they are bound, are joined to form a heterocyclic, a substituted heterocyclic, a heteroaryl or substituted heteroaryl group optionally containing from 1 to 3 additional hetero ring atoms selected from the group consisting of oxygen, nitrogen and sulfur;
R^{4″} is selected from the group consisting of hydrogen and alkyl;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl;
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enantiomers, diastereomers and pharmaceutically acceptable salts thereof.

7. The method of claim 1, wherein the steroid sparing compound is a compound of formula a compound of formula Va, Vb, Vc, or Vd: 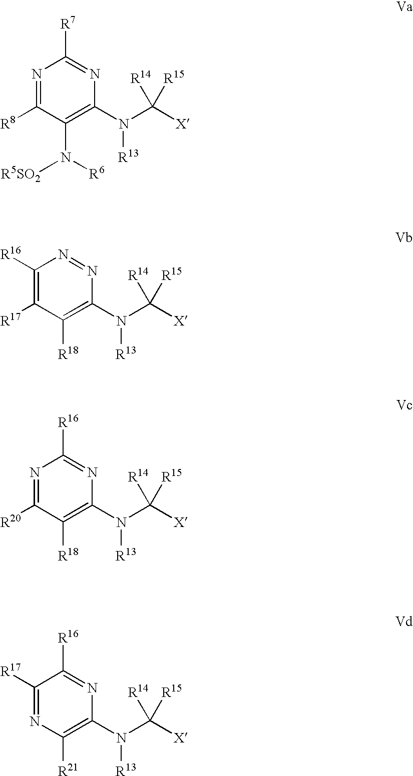
wherein:
R¹³ is selected from the group consisting of hydrogen, C₁₋₁₀ alkyl, Cy, and Cy-C₁₋₁₀ alkyl, wherein alkyl is optionally substituted with one to four substituents independently selected from R^{a}; and Cy is optionally substituted with one to four substituents independently selected from R^{b};
R¹⁴ is selected from the group consisting of hydrogen, C₁₋₁₀ alkyl, C₂₋₁₀ alkenyl, C₂₋₁₀ alkynyl, Cy, Cy-C₁₋₁₀ alkyl, Cy-C₂₋₁₀ alkenyl and Cy-C₂₋₁₀ alkynyl, wherein alkyl, alkenyl, and alkynyl are optionally substituted with one to four substituents selected from phenyl and R^{X}, and Cy is optionally substituted with one to four substituents independently selected from R¹;
or R¹³, R¹⁴ and the atoms to which they are attached together form a mono- or bicyclic ring containing 0-2 additional heteratoms selected from N, O and S;
R¹⁵ is selected from the group consisting of C₁₋₁₀ alkyl, C₂₋₁₀ alkenyl, C₂₋₁₀ alkynyl, aryl, aryl-C₁₋₁₀ alkyl, heteroaryl, heteroaryl-C₁₋₁₀ alkyl, wherein alkyl, alkenyl and alkynyl are optionally substituted with one to four substituents selected from R^{x}, and aryl and heteroaryl are optionally substituted with one to four substituents independently selected from R^{y};
or R¹⁴, R¹⁵ and the carbon to which they are attached form a 3-7 membered mono- or bicyclic ring containing 0-2 heteroatoms selected from N, O and S;
R^{a} is selected from the group consisting of Cy and a group selected from R^{x}, wherein Cy is optionally substituted with one to four substituents independently selected from R^{c};
R^{b} is selected from the group consisting of R^{a}, C₁₋₁₀ alkyl, C₂₋₁₀ alkenyl, C₂₋₁₀ alkynyl, aryl C₁₋₁₀alkyl, heteroaryl C₁₋₁₀ alkyl, wherein alkyl, alkenyl, alkynyl, aryl, heteroaryl are optionally substituted with a group independently selected from R^{c};
R^{c} is selected from the group consisting of halogen, NO₂, C(O)OR₁, C₁₋₄ alkyl, C₁₋₄ alkoxy, aryl, aryl C₁₋₄ alkyl, aryloxy, heteroaryl, NR^{f}R^{g}, R^{f}C(O)R^{g}, NR^{f}C(O)NR^{f}R^{g}, and CN;
R^{d} and R^{e} are independently selected from hydrogen, C₁₋₁₀ alkyl, C₂₋₁₀ alkenyl, C₂₋₁₀ alkynyl, Cy and Cy C₁₋₁₀alkyl, wherein alkyl, alkenyl, alkynyl and Cy are optionally substituted with one to four substituents independently selected from R^{c};
or R^{d} and R^{e} together with the atoms to which they are attached form a heterocyclic ring of 5 to 7 members containing 0-2 additional heteroatoms independently selected from oxygen, sulfur and nitrogen;
R^{f} and R^{g} are independently selected from hydrogen, C₁₋₁₀ alkyl, Cy and Cy-C₁₋₁₀ alkyl wherein Cy is optionally substituted with C₁₋₁₀ alkyl; or R^{f} and R^{g} together with the carbon to which they are attached form a ring of 5 to 7 members containing 0-2 heteroatoms independently selected from oxygen, sulfur and nitrogen;
R^{h} is selected from the group consisting of hydrogen, C₁₋₁₀ alkyl, C₂₋₁₀ alkenyl, C₂₋₁₀ alkynyl, cyano, aryl, aryl C₁₋₁₀ alkyl, heteroaryl, heteroaryl C₁₋₁₀ alkyl, and —SO₂Rⁱ; wherein alkyl, alkenyl, and alkynl are optionally substituted with one to four substitutents independently selected from R^{a}; and aryl and heteroaryl are each optionally substituted with one to four substituents independently selected from R^{b};
Rⁱ is selected from the group consisting of C₁₋₁₀ alkyl, C₂₋₁₀ alkenyl, C₂₋₁₀ alkynyl, and aryl; wherein alkyl, alkenyl, alkynyl and aryl are each optionally substituted with one to four substituents independently selected from R^{c};
R^{x} is selected from the group consisting of —OR^{d}, —NO₂, halogen, —S(O)ₘR^{d}, —SR^{d}, —S(O)₂OR^{d}, —S(O)ₘNR^{d}R^{e}, —NR^{d}R^{e}, O(CR^{f}R^{g})ₙNR^{d}R^{e}, —C(O)R^{d}, —CO₂R^{d}, —CO₂(CR^{f}R^{g})ₘCONR^{d}R^{e}, —OC(O)R^{d}, —CN, —C(O)NR^{d}R^{e}, —NR^{d}C(O)R^{e}, —OC(O)NR^{d}R^{e}, —NR^{d}C(O)OR^{e}, —NR^{d}C(O)NR^{d}R^{e}, —CR^{d}(N—OR^{e}), CF₃, oxo, NR^{d}C(O)NR^{d}SO₂Rⁱ, NR^{d}S(O)ₘR^{e}, —OS(O)₂OR^{d}, and —OP(O)(OR^{d})₂;
R^{y} is selected from the group consisting of R^{x}, C₁₋₁₀ alkyl, C₂₋₁₀ alkenyl, C₂₋₁₀ alkynyl, aryl C₁₋₁₀alkyl, heteroaryl C₁₋₁₀ alkyl, cycloalkyl, heterocyclyl; wherein alkyl, alkenyl, alkynyl and aryl are each optionally substituted with one to four substitutents independently selected from R^{x};
Cy is cycloalkyl, heterocyclyl, aryl, or heteroaryl;
m is an integer from 1 to 2;
n is an integer from 1 to 10;
X′ is selected from the group consisting of —C(O)OR^{d}, P(O)(OR^{d})(OR^{e}), —P(O)(R^{d})(OR^{e}), —S(O)ₘOR^{d}, C(O)NR^{d}R^{h}, and -5-tetrazolyl;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl; and
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enatiomers, diastereomers and pharmaceutically acceptable salts thereof.

8. The method of claim 1, wherein the steroid sparing compound is a compound of formula VIa, VIb, VIc, or VId: 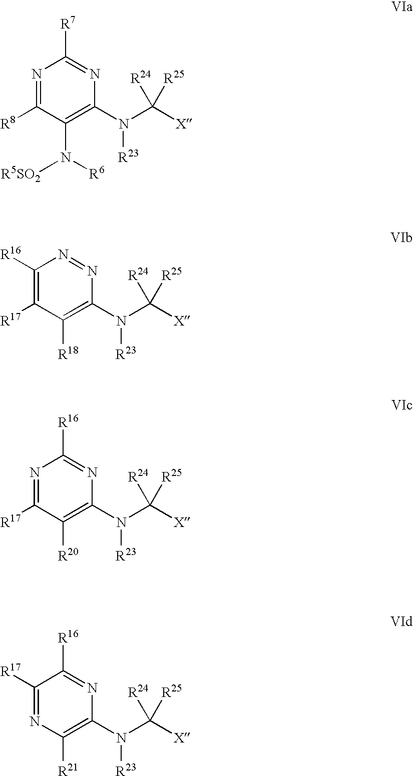
wherein:
R²³ is selected from the group consisting of hydrogen, C₁₋₁₀ alkyl optionally substituted with one to four substituents independently selected from R^{a′} and Cy optionally substituted with one to four substituents independently selected from R^{b′};
R²⁴ is selected from the group consisting of Ar¹—Ar²—C₁₋₁₀ alkyl, Ar¹—Ar²—C₂₋₁₀ alkenyl, Ar¹—Ar²—C₂₋₁₀ alkynyl, wherein Ar¹ and Ar² are independently aryl or heteroaryl each of which is optionally substituted with one to four substituents independently selected from R^{b′}; alkyl, alkenyl and alkynyl are optionally substituted with one to four substituents independently selected from R^{a};
R²⁵ is selected from the group consisting of hydrogen, C₁₋₁₀alkyl, C₂₋₁₀ alkenyl, C₂₋₁₀ alkynyl, aryl, aryl C₁₋₁₀alkyl, heteroaryl, and heteroaryl C₁₋₁₀alkyl, wherein alkyl, alkenyl and alkynyl are optionally substituted with one to four substituents selected from R^{a′}, and aryl and heteroaryl are optionally substituted with one to four substituents independently selected from R^{b′};
R^{a′} is selected from the group consisting of Cy, —OR^{d}, —NO₂, halogen —S(O)ₘR^{d′}, —SR^{d′}, —S(O)₂OR^{d′}, —S(O)ₘNR^{d′}, —S(O)ₘNR^{d′}R_{e′}, —NR^{d′}R^{e′}, —O(CR^{f′}R^{g′})ₙNR^{d′}R^{e′}, —C(O)R^{d′}, —CO₂R^{d′}, —CO₂(CR^{f′}R^{g′})ₙCONR^{d′}R^{e′}, —OC(O)R^{d′}, —CN, —C(O)NR^{d′}R^{e′}, NR^{d′}C(O)R^{e′}, —OC(O)NR^{d′}R^{e′}, —NR^{d′}C(O)OR^{e′}, —NR^{d′}C(O)NR^{d′}R^{e′}, CR^{d′}(N—OR^{e′}) CF₃, and —OCF₃;
wherein Cy is optionally substituted with one to four substituents independently selected from R^{c′};
R^{b} is selected from the group consisting of R^{a′}, C₁₋₁₀ alkyl, C₂₋₁₀ alkenyl, C₂₋₁₀ alkynyl, aryl C₁₋₁₀alkyl, heteroaryl C₁₋₁₀alkyl,
wherein alkyl, alkenyl, aryl, heteroaryl are optionally substituted with a group independently selected from R^{c′};
R^{c′} is selected from the group consisting of halogen, amino, carboxy, C₁₋₄ alkyl, C₁₋₄ alkoxy, aryl, aryl C₁₋₄₋alkyl, hydroxy, CF₃, and aryloxy;
R^{d′} and R^{e′} are independently selected from hydrogen, C₁₋₁₀ alkyl, C₂₋₁₀ alkenyl, C₂₋₁₀ alkynyl, Cy and Cy C₁₋₁₀alkyl, wherein alkyl, alkenyl, alkynyl and Cy are optionally substituted with one to four substituents independently selected from R^{c′}; or R^{d′} and R^{e′} together with the atoms to which they are attached form a heterocyclic ring of 5 to 7 members containing 0-2 additional heteroatoms independently selected from oxygen, sulfur and nitrogen;
R^{f′} and R^{g′} are independently selected from hydrogen, C₁₋₁₀ alkyl, Cy and Cy-C₁₋₁₀ alkyl; or R^{f′} and R^{g′} together with the carbon to which they are attached form a ring of 5 to 7 members containing 0-2 heteroatoms independently selected from oxygen, sulfur and nitrogen;
R^{h′} is selected from the group consisting of hydrogen, C₁₋₁₀ alkyl, C₂₋₁₀ alkenyl, C₂₋₁₀ alkynyl, cyano, aryl, aryl C₁₋₁₀ alkyl, heteroaryl, heteroaryl C₁₋₁₀ alkyl, or —SO₂R^{i′};
wherein alkyl, alkenyl, and alkynyl are optionally substituted with one to four substitutents independently selected from R^{a′}; and aryl and heteroaryl are each optionally substituted with one to four substituents independently selected from R^{b′};
R^{i′} is selected from the group consisting of C₁₋₁₀ alkyl, C₂₋₁₀ alkenyl, C₂₋₁₀ alkynyl, and aryl;
wherein alkyl, alkenyl, alkynyl and aryl are each optionally substituted with one to four substituents independently selected from R^{c′};
Cy is cycloalkyl, heterocyclyl, aryl, or heteroaryl;
X″ is selected from the group consisting of —C(O)OR^{d′}, —P(O)(OR^{d′})(OR^{e′}), —P(O)(R^{d′})(OR^{e′}), —S(O)ₘOR^{d}, —C(O)NR^{d′}R^{h′}, and -5-tetrazolyl;
m is an integer from 1 to 2;
n is an integer from 1 to 10;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl; and
R⁷ and R⁸ are independently selected from the group consisting of hydrogen, alkyl, substituted alkyl, cycloalkyl, substituted cycloalkyl, aryl, substituted aryl, heteroaryl, substituted heteroaryl, heterocyclic, substituted heterocyclic and halogen;
R¹⁶ and R¹⁷ are independently selected from the group consisting of hydrogen, alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic, substituted heterocyclic and halogen; and
R¹⁸ is selected from the group consisting of alkyl, substituted alkyl, alkoxy, substituted alkoxy, amino, substituted amino, cycloalkyl, substituted cycloalkyl, aryl, substituted aryl, heteroaryl, substituted heteroaryl, heterocyclic and substituted heterocyclic;
R²⁰ is selected from the group consisting of hydrogen, alkyl, substituted alkyl, alkoxy, substituted alkoxy, cycloalkyl, substituted cycloalkyl, aryl, substituted aryl, heteroaryl, substituted heteroaryl, heterocyclic, substituted heterocyclic and halogen;
R²¹ is selected from the group consisting of alkyl, substituted alkyl, alkoxy, substituted alkoxy, amino, substituted amino, cycloalkyl, substituted cycloalkyl, aryl, substituted aryl, heterocyclic and substituted heterocyclic;
and enantiomers, diastereomers and pharmaceutically acceptable salts thereof.

9. The method of claim 1, wherein the steroid sparing compound is a compound of formula VII: 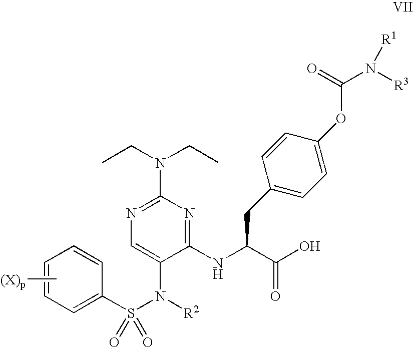
wherein each X is independently fluoro, chloro or bromo;
p is an integer from 0 to 3;
R¹ and R³ together with the nitrogen atom to which they are bound form an azetidinyl, pyrrolidinyl, pyrrolyl, 2,5-dihydopyrrol-1-yl, piperidinyl, or 1,2,3,6-tetrahydropyridin-1-yl;
R² is selected from the group consisting of lower alkyl, lower alkenyl, and lower alkylenecycloalkyl;
and pharmaceutically acceptable salts thereof.

10. The method of claim 1, wherein the steroid sparing compound is a compound of formula a compound of formula IX: 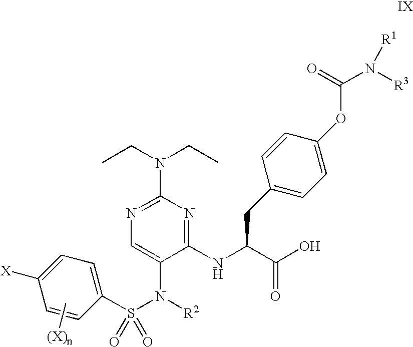
wherein each X is independently fluoro or chloro;
n is zero or one;
R² is —CH₂—R′ where R′ is selected from the group consisting of hydrogen, methyl or —CH═CH₂;
R¹ and R³ together with the nitrogen atom to which they are bound form an azetidinyl, pyrrolidinyl, or piperidinyl group;
and pharmaceutically acceptable salts thereof.

11. The method of claim 1, wherein the steroid sparing compound is a compound of formula a compound of formula X: 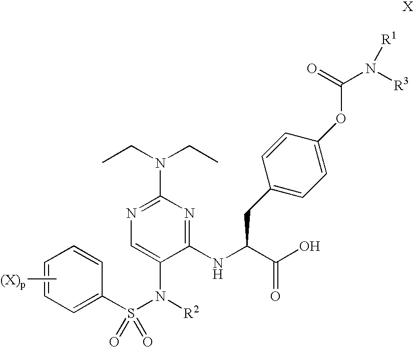
wherein each X is independently fluoro, chloro or bromo;
p is an integer from 0 to 3;
R¹ and R³ together with the nitrogen atom to which they are bound form an azetidinyl, pyrrolidinyl, pyrrolyl, 2,5-dihydopyrrol-1-yl, piperidinyl, or 1,2,3,6-tetrahydropyridin-1-yl;
R² is lower alkynyl;
and pharmaceutically acceptable salts thereof.

12. The method of claim 1, wherein the steroid sparing compound is a compound of formula a compound of formula XIII or XIV:

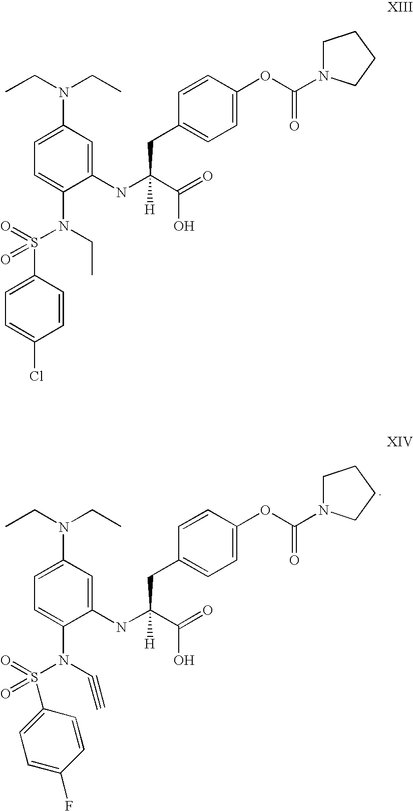

13. The method of claim 1, wherein the steroid sparing compound is a compound of formula a compound of formula XV: 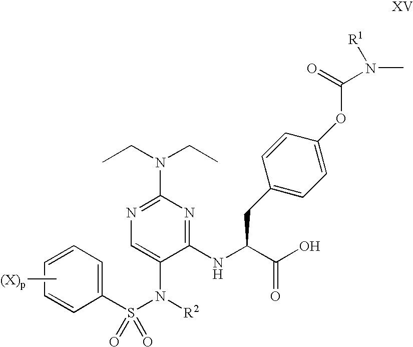
wherein each X is independently fluoro, chloro or bromo;
p is 0 or an integer from 1-3;
R¹ is selected from the group consisting of methyl and ethyl;
R² is selected from the group consisting of lower alkyl, lower alkenyl, and lower alkylenecycloalkyl;
and pharmaceutically acceptable salts thereof.

14. The method of claim 1, wherein the steroid sparing compound is a compound of formula a compound of formula XVIII: 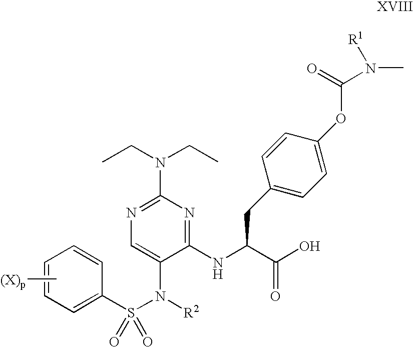
wherein each X is independently fluoro, chloro or bromo;
p is 0 or an integer from 1-3;
R¹ is selected from the group consisting of methyl and ethyl;
R² is lower alkynyl;
and pharmaceutically acceptable salts thereof.

15. The method of claim 1, wherein the steroid sparing compound is a compound of formula a compound of formula XXI: 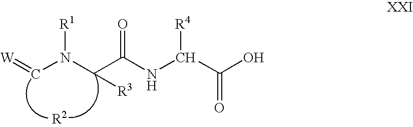
wherein:
R¹ is selected from the group consisting of alkyl, substituted alkyl, aryl, substituted aryl, cycloalkyl, substituted cycloalkyl, heterocyclic, substituted heterocylic, heteroaryl, substituted heteroaryl and —C(O)OR¹;
R² is selected from the group consisting of alkylene having from 2 to 4 carbon atoms in the alkylene chain, substituted alkylene having from 2 to 4 carbon atoms in the alkylene chain, heteroalkylene containing from 1 to 3 carbon atoms and from 1 to 2 heteroatoms selected from nitrogen, oxygen and sulfur and having from 2 to 4 atoms in the heteroalkylene chain, and substituted heteroalkylene containing, in the heteroalkylene chain, from 1 to 3 carbon atoms and from 1 to 2 heteroatoms selected from nitrogen, oxygen and sulfur and having from 2 to 4 atoms in the heteroalkylene chain;
R³ is selected from the group consisting of hydrogen, alkyl, substituted alkyl, cycloalkyl, substituted cycloalkyl, cycloalkenyl, substituted cycloalkenyl, aryl, substituted aryl, heteroaryl, substituted heteroaryl, heterocyclic, substituted heterocyclic; or R³ can be joined to R² to form a fused cycloalkyl, substititued cycloalkyl, cycloalkenyl, substituted cycloalkenyl, heterocyclic or substituted heterocyclic ring;
R⁴ is selected from the group consisting of isopropyl, —CH₂—X and ═CH—X, where X is selected from the group consisting of hydrogen, alkyl, substituted alkyl, cycloalkyl, substituted cycloalkyl, alkoxy, substituted alkoxy, aryl, substituted aryl, aryloxy, substituted aryloxy, aryloxyaryl, substituted aryloxyaryl, heteroaryl, substituted heteroaryl, heterocyclic, substituted heterocyclic, acylamino, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxyheterocyclic, carboxy-substituted heterocyclic, and hydroxyl with the proviso that when R⁴ is ═CH—X then (H) is removed from the formula and X is not hydroxyl;
W is oxygen or sulfur;
and pharmaceutically acceptable salts thereof.

16. The method of claim 1, wherein the steroid sparing compound is a compound of formula a compound of formula XXIa: 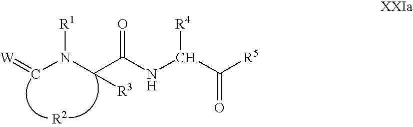
wherein:
R¹ is selected from the group consisting of alkyl, substituted alkyl, aryl, substituted aryl, cycloalkyl, substituted cycloalkyl, heterocyclic, substituted heterocylic, heteroaryl, substituted heteroaryl and —C(O)OR¹;
R² is selected from the group consisting of alkylene having from 2 to 4 carbon atoms in the alkylene chain, substituted alkylene having from 2 to 4 carbon atoms in the alkylene chain, heteroalkylene containing from 1 to 3 carbon atoms and from 1 to 2 heteroatoms selected from nitrogen, oxygen and sulfur and having from 2 to 4 atoms in the heteroalkylene chain, and substituted heteroalkylene containing, in the heteroalkylene chain, from 1 to 3 carbon atoms and from 1 to 2 heteroatoms selected from nitrogen, oxygen and sulfur and having from 2 to 4 atoms in the heteroalkylene chain;
R³ is selected from the group consisting of hydrogen, alkyl, substituted alkyl, cycloalkyl, substituted cycloalkyl, cycloalkenyl, substituted cycloalkenyl, aryl, substituted aryl, heteroaryl, substituted heteroaryl, heterocyclic, substituted heterocyclic; or R³ can be joined to R² to form a fused cycloalkyl, substititued cycloalkyl, cycloalkenyl, substituted cycloalkenyl, heterocyclic or substituted heterocyclic ring;
R⁴ is selected from the group consisting of isopropyl, —CH₂—X and ═CH—X, where X is selected from the group consisting of hydrogen, alkyl, substituted alkyl, cycloalkyl, substituted cycloalkyl, alkoxy, substituted alkoxy, aryl, substituted aryl, aryloxy, substituted aryloxy, aryloxyaryl, substituted aryloxyaryl, heteroaryl, substituted heteroaryl, heterocyclic, substituted heterocyclic, acylamino, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxyheterocyclic, carboxy-substituted heterocyclic, and hydroxyl with the proviso that when R⁴ is ═CH—X then (H) is removed from the formula and X is not hydroxyl;
R⁵ is selected from the group consisting of amino, alkoxy, substituted alkoxy, cycloalkoxy, substituted cycloalkoxy, aryloxy, substituted aryloxy, heteroaryloxy, substituted heteroaryloxy, heterocyclyloxy, substituted heterocyclyloxy, —NHOY where Y is hydrogen, alkyl, substituted alkyl, aryl, or substituted aryl, and —NH(CH₂)ₚCOOY′ where Y′ is hydrogen, alkyl, substituted alkyl, aryl, or substituted aryl, and p is an integer of from 1 to 8;
W is oxygen or sulfur;
and pharmaceutically acceptable salts thereof;
with the provisos that:
(a) when R¹ is benzyl, R² is —CH₂CH₂—, R³ is hydrogen, R⁴ is benzyl, then R⁵ is not ethyl;
(b) when R¹ is 3,4-dichlorobenzyl, R² is —CH₂CH₂—, R³ is hydrogen, R⁴ is 4-(phenylcarbonylamino)benzyl, then R⁵ is not methyl;
(c) when R¹ is benzyl, R² is —CH₂CH₂—, R³ is hydrogen, R⁴ is 4-hydroxybenzyl, then R⁵ is not isopropyl or tert-butyl;
(d) when R¹ is 4-flurobenzyl, R² is —CH₂CH₂—, R³ is hydrogen, R⁵ is tert-butyl, then R⁴ is not 4-hydroxybenzyl or 4-(4-nitrophenoxy-carbonyloxy)benzyl;
(e) when R¹ is 4-cyanobenzyl, R² is —CH₂CH₂—, R³ is hydrogen, R⁴ is 4-hydroxybenzyl, then R⁵ is not tert-butyl; and
(f) when R¹ is benzyloxycarbonyl, R² is —NHCH₂—, R³ is hydrogen, R⁵ is tert-butyl, then R⁴ is not 4-hydroxybenzyl or 4-(N,N-dimethylcarbamyloxy)benzyl.

17. The method of claim 1, wherein the subject is a human.

18. The method of claim 1, wherein the compound is administered parenterally.

19. The method of claim 1, wherein the compound is administered chronically to the subject in need thereof.

20. The method of claim 19, wherein the chronic administration of the compound is weekly or monthly over a period of at least one year.

21. The method of claim 1, wherein the compound is administered intravenously in an amount of 0.5 mg to about 100 mg per kilogram body weight of the subject.

22. The method of claim 1, wherein the disease is inflammatory bowel disease and wherein the steroid sparing effective amount permits the subject to be tapered from steroid therapy.

23. The method of claim 22, wherein the inflammatory bowel disease is selected from the group consisting of Crohn's disease and ulcerative colitis.

24. The method of claim 22, wherein the compound is administered parenterally.

25. The method of claim 22, wherein the subject is refractory, intolerant or dependent on steroids.

26. The method of claim 1, wherein the subject requires a therapeutically effective amount of steroids that is less than would be required in the absence of administering the agent.

27. A combination therapy for the treatment of a disease, selected from the group consisting of inflammatory bowel disease, asthma, multiple schlerosis, rheumatoid arthritis, graft vs. host disease, host vs. graft disease, spondyloarthropathies, and combinations thereof, comprising a steroid sparing effective amount of a first steroid sparing agent and a second agent selected from the group consisting of
(i) an immunosuppressant, wherein the immunosuppressant is not a steroid;
(ii) an anti-TNF composition;
(iii) a 5-ASA composition; and
(iv) combinations thereof.

28. The combination therapy of claim 27, wherein the combination therapy comprises a therapeutically effective amount of a second steroid sparing agent.

29. The combination therapy of claim 27, wherein the second steroid sparing agent is an antibody or an immunologically active fragment thereof.

30. The combination therapy of claim 29, wherein the second steroid sparing agent is an antibody or an immunologically active fragment thereof that binds to α₄β1 integrin and/or α₄β7 integrin.

31. The combination therapy of claim 30, wherein the second steroid sparing agent is natalizumab.

32. The combination therapy of claim 27, wherein the immunosuppressant is selected from the group consisting of azathioprine, 6-mercaptopurine, methotrexate, and mycophenolate.

33. The combination therapy of claim 27, wherein the anti-TNF composition is infliximab.

34. The combination therapy of claim 27, wherein the 5-ASA agent is selected from the group consisting of mesalazine and osalazine.

35. The method of claim 1, wherein the disease is multiple sclerosis and wherein the steroid sparing effective amount permits the subject to be tapered from steroid therapy.

36. The method of claim 35, wherein the steroid sparing agent is a compound of formula I: 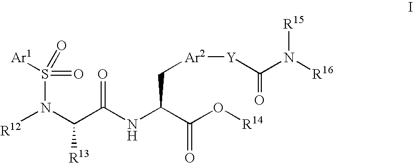
wherein:
Ar¹ is selected from the group consisting of aryl, substituted aryl, heteroaryl, and substituted heteroaryl;
Ar² is selected from the group consisting of aryl, substituted aryl, heteroaryl and substituted heteroaryl;
R¹² and R¹³ together with the nitrogen atom bound to R¹² and the carbon atom bound to R¹³ form a heterocyclic or substituted heterocyclic group;
R¹⁴ is selected from the group consisting of hydrogen, alkyl, substituted alkyl, cycloalkyl, substituted cycloalkyl, aryl, and substituted aryl;
R¹⁵ is selected from the group consisting of alkyl, and substituted alkyl, or R¹⁵ and R¹⁶ together with the nitrogen atom to which they are bound form a heterocyclic or substituted heterocyclic group;
R¹⁶ is selected from the group consisting of alkyl and substituted alkyl or R¹⁵ and R¹⁶ together with the nitrogen atom to which they are bound form a heterocyclic or substituted heterocyclic group; and
Y is selected from the group consisting of —O— and —NR¹⁰⁰—, wherein R¹⁰⁰ is hydrogen or alkyl;
and pharmaceutically acceptable salts thereof.

37. The method of claim 35, wherein the subject requires a therapeutically effective amount of steroids that is less than would be required in the absence of administering the compound.

38. The method of claim 35, wherein the subject is refractory, intolerant or dependent on steroids.

39. The method of claim 1, wherein the disease is rheumatoid arthritis and wherein the steroid sparing effective amount permits the subject to be tapered from steroid therapy.

40. The method of claim 39, wherein the steroid sparing agent is a compound of formula VII: 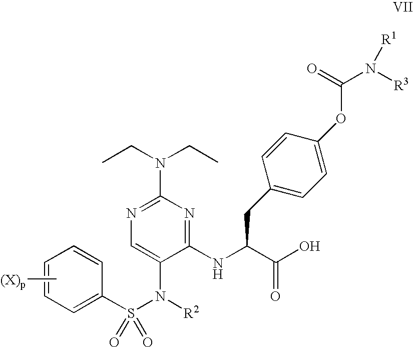
wherein each X is independently fluoro, chloro or bromo;
p is an integer from 0 to 3;
R¹ and R³ together with the nitrogen atom to which they are bound form an azetidinyl, pyrrolidinyl, pyrrolyl, 2,5-dihydopyrrol-1-yl, piperidinyl, or 1,2,3,6-tetrahydropyridin-1-yl;
R² is selected from the group consisting of lower alkyl, lower alkenyl, and lower alkylenecycloalkyl;
and pharmaceutically acceptable salts thereof.

41. The method of claim 39, wherein the steroid sparing agent is a compound of formula XV: 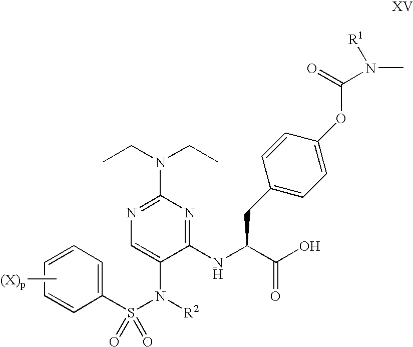
wherein each X is independently fluoro, chloro or bromo;
p is 0 or an integer from 1-3;
R¹ is selected from the group consisting of methyl and ethyl;
R² is selected from the group consisting of lower alkyl, lower alkenyl, and lower alkylenecycloalkyl;
and pharmaceutically acceptable salts thereof.

42. The method of claim 39, wherein the subject requires a therapeutically effective amount of steroids that is less than would be required in the absence of administering the compound.

43. The method of claim 39, wherein the subject is refractory, intolerant or dependent on steroids.

44. The method of claim 1, wherein the disease is host versus graft or graft versus host and wherein the steroid sparing effective amount permits the subject to be tapered from steroid therapy.

45. The method of claim 44, wherein the subject requires a therapeutically effective amount of steroids that is less than would be required in the absence of administering the compound.

46. The method of claim 44, wherein the subject is refractory, intolerant or dependent on steroids.

47. The method of claim 1, wherein the disease is asthma and wherein the steroid sparing effective amount permits the subject to be tapered from steroid therapy.

48. The method of claim 47, wherein the steroid sparing agent is a compound of formula I: 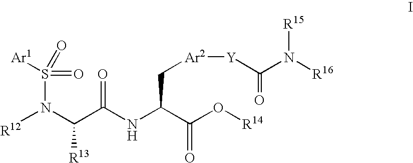
wherein:
Ar¹ is selected from the group consisting of aryl, substituted aryl, heteroaryl, and substituted heteroaryl;
Ar² is selected from the group consisting of aryl, substituted aryl, heteroaryl and substituted heteroaryl;
R¹² and R¹³ together with the nitrogen atom bound to R¹² and the carbon atom bound to R¹³ form a heterocyclic or substituted heterocyclic group;
R¹⁴ is selected from the group consisting of hydrogen, alkyl, substituted alkyl, cycloalkyl, substituted cycloalkyl, aryl, and substituted aryl;
R¹⁵ is selected from the group consisting of alkyl, and substituted alkyl, or R¹⁵ and R¹⁶ together with the nitrogen atom to which they are bound form a heterocyclic or substituted heterocyclic group;
R¹⁶ is selected from the group consisting of alkyl and substituted alkyl or R¹⁵ and R¹⁶ together with the nitrogen atom to which they are bound form a heterocyclic or substituted heterocyclic group; and
Y is selected from the group consisting of —O— and —NR¹⁰⁰—, wherein R¹⁰⁰ is hydrogen or alkyl;
and pharmaceutically acceptable salts thereof.

49. The method of claim 47, wherein the steroid sparing agent is a compound of formula II: 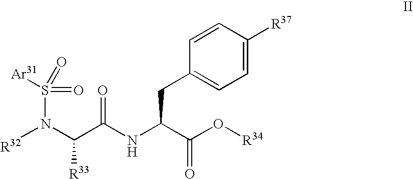
wherein:
Ar³¹ is selected from the group consisting of aryl, substituted aryl, heteroaryl, and substituted heteroaryl;
R³² and R³³ together with the nitrogen atom bound to R³² and the carbon atom bound to R³³ form a heterocyclic or substituted heterocyclic group;
R³⁴ is selected from the group consisting of hydrogen, alkyl, substituted alkyl, cycloalkyl, substituted cycloalkyl, aryl, and substituted aryl; and
R³⁷ is aryl, heteroaryl, substituted aryl, substituted heteroaryl, heterocyclic, substituted heterocyclic, aryloxy, substituted aryloxy, aralkoxy, substituted aralkoxy, heteroaryloxy, substituted heteroaryloxy;
and pharmaceutically acceptable salts thereof.

50. The method of claim 47, wherein the steroid sparing agent is a compound of formula IIIa or formula IIIb: 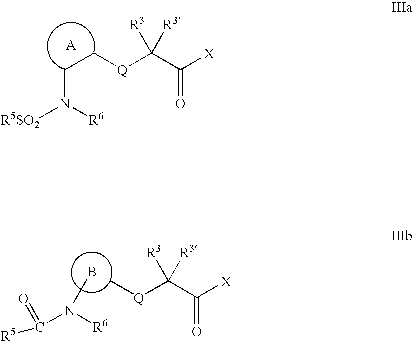
wherein:
R³ and R^{3′} are independently selected from the group consisting of hydrogen, isopropyl, —CH₂Z where Z is selected from the group consisting of hydrogen, hydroxyl, acylamino, alkyl, alkoxy, aryloxy, aryl, aryloxyaryl, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted alkyl, substituted alkoxy, substituted aryl, substituted aryloxy, substituted aryloxyaryl, substituted cycloalkyl, heteroaryl, substituted heteroaryl, heterocyclic and substituted heterocyclic, or
R³ and R^{3′} are joined to form a substituent selected from the group consisting of ═CHZ where Z is defined above provided that Z is not hydroxyl or thiol, cycloalkyl, substituted cycloalkyl, cycloalkenyl, substituted cycloalkenyl, heterocyclic and substituted heterocyclic;
X is selected from the group consisting of hydroxyl, alkoxy, substituted alkoxy, alkenoxy, substituted alkenoxy, cycloalkoxy, substituted cycloalkoxy, cycloalkenoxy, substituted cycloalkenoxy, aryloxy, substituted aryloxy, heteroaryloxy, substituted heteroaryloxy, heterocyclyloxy, substituted heterocyclyloxy and —NR″R″ where each R″ is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic and substituted heterocyclic;
Q is selected from the group consisting of —O—, —S—, —S(O)—, —S(O)₂—, and —NR⁴—;
ring A and ring B independently form a heteroaryl or substituted heteroaryl group having two nitrogen atoms in the heteroaryl ring;
R⁴ is selected from the group consisting of hydrogen, alkyl, substituted alkyl, alkenyl, substituted alkenyl, cycloalkyl, substituted cycloalkyl, cycloalkenyl, substituted cycloalkenyl, aryl, substituted aryl, heteroaryl, substituted heteroaryl, heterocyclic and substituted heterocyclic;
R⁵ is selected from the group consisting of alkyl, substituted alkyl, alkenyl, substituted alkenyl, aryl, substituted aryl, cycloalkyl, substituted cycloalkyl, cycloalkenyl, substituted cycloalkenyl, heterocyclic, substituted heterocylic, heteroaryl and substituted heteroaryl;
R⁶ is selected from the group consisting of hydrogen, alkyl, substituted alkyl, cycloalkyl, substituted cycloalkyl, cycloalkenyl, substituted cycloalkenyl, heterocyclic, substituted heterocyclic, aryl, substituted aryl, heteroaryl, substituted heteroaryl, and —SO₂R¹⁰ where R¹⁰ is selected from the group consisting of alkyl, substituted alkyl, cycloalkyl, substituted cycloalkyl, cycloalkenyl, substituted cycloalkenyl, heterocyclic, substituted heterocyclic, aryl, substituted aryl, heteroaryl, and substituted heteroaryl;
or optionally, one of, R⁴ and ring A, R⁴ and R⁵, R⁴ and R⁶, or R⁵ and R, together with the atoms to which they are bound, can be joined to form a heterocyclic or substituted heterocyclic ring;
provided that ring B does not form a 6-amino or substituted amino pyrimidin-4-yl group;
and enantiomers, diastereomers and pharmaceutically acceptable salts thereof.

51. The method of claim 47, wherein the subject requires a therapeutically effective amount of steroids that is less than would be required in the absence of administering the compound.

52. The method of claim 47, wherein the subject is refractory, intolerant or dependent on steroids.

53. The method of claim 1, wherein the disease is spondyloarthropathies and wherein the steroid sparing effective amount permits the subject to be tapered from steroid therapy.

54. The method of claim 53, wherein the subject requires a therapeutically effective amount of steroids that is less than would be required in the absence of administering the compound.

55. The method of claim 53, wherein the spondyloarthropathies are selected from the group consisting of ankylosing spondylitis, psoriatic arthritis, Reiter's Syndrome, spondylitis of inflammatory bowel disease, undifferentiated spondyloarthropathy, and juvenile spondylarthropathy.

56. The method of claim 53, wherein the subject is refractory, intolerant or dependent on steroids.
